(19) United States
(12) Patent Application Publication
(10) Pub. No.: US 2015/0158851 A1
(43) Pub. Date: Jun. 11, 2015

(54) PYRAZOLE CARBOXAMIDE COMPOUNDS, COMPOSITIONS AND METHODS OF USE

(71) Applicant: Genentech, Inc., South San Francisco, CA (US)

(72) Inventors: Jason Burch, Redwood City, CA (US); Frederick Brookfield, Benson (GB); Richard A. Goldsmith, Belmont, CA (US); Kevin Hon Luen Lau, San Mateo, CA (US); Colin H. MacKinnon, Oxfordshire (GB); Daniel Fred Ortwine, San Ramon, CA (US); Zhonghua Pei, Burlingame, CA (US)

(73) Assignee: Genentech, Inc., South San Francisco, CA (US)

(21) Appl. No.: 14/618,827

(22) Filed: Feb. 10, 2015

Related U.S. Application Data
(63) Continuation of application No. PCT/CN2013/081136, filed on Aug. 9, 2013.
(60) Provisional application No. 61/682,063, filed on Aug. 10, 2012.
(60) Provisional application No. 61/764,434, filed on Feb. 13, 2013.
(60) Provisional application No. 61/764,930, filed on Feb. 14, 2013.

(51) Int. Cl. C07D 403/12; C07D 491/107; C07D 401/14; C07D 405/14; C07D 487/10; C07D 413/14; C07D 491/044; C07D 417/14; C07D 495/10; C07D 403/14; C07D 409/14

(52) U.S. Cl. CPC C07D 403/12; C07D 403/14; C07D 491/107; C07D 401/14; C07D 405/14; C07D 409/14; C07D 413/14; C07D 491/044; C07D 417/14; C07D 495/10; C07D 487/10

(57) ABSTRACT

Provided herein are compounds of formula (AA):

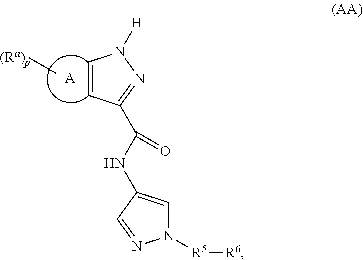

stereoisomers or a pharmaceutically acceptable salt thereof, wherein A, R^{a}, p, R⁵ and R⁶ are defined herein, compositions including the compounds and methods of manufacturing and using the compounds for the treatment of diseases.

This application is a continuation of International Application No. PCT/CN2013/081136, filed Aug. 9, 2013, which claims the benefit of priority to U.S. Provisional Application Ser. No. 61/682,063, filed Aug. 10, 2012, U.S. Provisional Application Ser. No. 61/764,434, filed Feb. 13, 2013, and U.S. Provisional Application Ser. No. 61/764,930, filed Feb. 14, 2013, each of which is incorporated herein by reference in its entirety.

### FIELD OF THE INVENTION

Compounds of the present invention, which are inhibitors of ITK kinase, as well as compositions containing these compounds, and methods of use including, but not limited to, in vitro, in situ and in vivo diagnosis or treatment of mammalian cells are provided herein. Exemplary conditions that can be treated with such compounds include cancer and asthma.

### BACKGROUND OF THE INVENTION

ITK is a Tec family kinase that is expressed in T cells, NKT cells, NK cells, and mast cells. ITK is activated downstream of antigen engagement of the T cell receptor (TCR) and mediates TCR signals through the phosphorylation and activation of PLCg. Mice in which ITK is deleted showed defective differentiation of T cells towards the Th2 subset, but not the Th1 subset. Additional studies indicate that Th2 cytokine production, but not early Th2 lineage commitment, is defective in ITK-deficient mouse T cells. Th2 cells promote allergic inflammation, and ITK knock-out mice have reduced lung inflammation, mucus production, and airway hyperreactivity in models of allergic asthma. The reduction in lung pathology in ITK knock-out asthma models is not rescued by a kinase-deficient ITK transgene, indicating that the kinase activity of ITK is necessary for asthma pathology. Human patients with immunological and inflammatory disorders, such as the allergic disease atopic dermatitis, express higher levels of ITK in peripheral blood T cells.

There exists a need for inhibitors of ITK kinase and treatments of diseases and disorders mediated by ITK kinase.

### SUMMARY OF THE INVENTION

An aspect includes a compound of formula (AA):

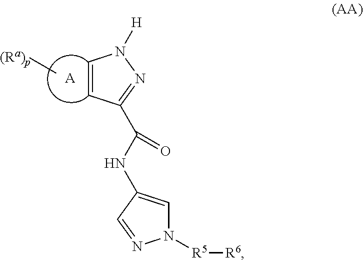

stereoisomers or a pharmaceutically acceptable salt thereof, wherein ring A, R^{a}, p, R⁵ and R⁶ are defined herein.

Another aspect includes a pharmaceutical composition comprising a compound of the present invention, stereoisomers or a pharmaceutically acceptable salt thereof and a therapeutically inert carrier, diluent or excipient.

Another aspect includes a method of treating a disease responsive to the inhibition of ITK kinase in a patient, comprising administering an effective amount of a compound of the present invention, stereoisomers or a pharmaceutically acceptable salt thereof.

Another aspect includes a method of treating an immunological or inflammatory disease in a patient, comprising administering an effective amount of a compound of the present invention, stereoisomers or a pharmaceutically acceptable salt thereof.

Another aspect includes the use of a compound of the present invention, stereoisomers or a pharmaceutically acceptable salt thereof in therapy.

Another aspect includes the use of a compound of the present invention, stereoisomers or a pharmaceutically acceptable salt thereof in the treatment of a disease responsive to the inhibition of ITK kinase.

Another aspect includes the use of a compound of the present invention, stereoisomers or a pharmaceutically acceptable salt thereof in the treatment of an immunological or inflammatory disease.

### DETAILED DESCRIPTION OF THE INVENTION

### Definitions

“Acyl” means a carbonyl containing substituent represented by the formula —C(O)—R in which R is hydrogen, alkyl, a cycloalkyl, a heterocyclyl, cycloalkyl-substituted alkyl or heterocyclyl-substituted alkyl wherein the alkyl, alkoxy, cycloalkyl and heterocyclyl are as defined herein. Acyl groups include alkanoyl (e.g., acetyl), aroyl (e.g., benzoyl), and heteroaroyl (e.g., pyridinoyl).

The term “alkyl” refers to a saturated linear or branched-chain monovalent hydrocarbon radical, wherein the alkyl radical may be optionally substituted independently with one or more substituents described herein. In one example, the alkyl radical is one to eighteen carbon atoms (C₁-C₁₈). In other examples, the alkyl radical is C₀-C₆, C₀-C₅, C₀-C₃, C₁-C₁₂, C₁-C₁₀, C₁-C₈, C₁-C₆, C₁-C₅, C₁-C₄ or C₁-C₃. C₀ alkyl refers to a bond. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂ CH₂ CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl and 1-octyl.

The term “alkenyl” refers to a linear or branched-chain monovalent hydrocarbon radical with at least one site of unsaturation, i.e., a carbon-carbon double bond, wherein the alkenyl radical may be optionally substituted independently with one or more substituents described herein, and includes radicals having “cis” and “trans” orientations, or alternatively, “E” and “Z” orientations. In one example, the alkenyl radical is two to eighteen carbon atoms (C₂-C₁₈). In other examples, the alkenyl radical is C₂-C₁₂, C₂-C₁₀, C₂-C₈, C₂-C₆ or C₂-C₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The term “alkoxy” refers to a linear or branched monovalent radical represented by the formula —OR in which R is alkyl, alkenyl, alkynyl or cycloalkyl, which can be further optionally substituted as defined herein. Alkoxy groups include methoxy, ethoxy, propoxy, isopropoxy, mono-, di- and tri-fluoromethoxy and cyclopropoxy.

The term “alkynyl” refers to a linear or branched monovalent hydrocarbon radical with at least one site of unsaturation, i.e., a carbon-carbon, triple bond, wherein the alkynyl radical may be optionally substituted independently with one or more substituents described herein. In one example, the alkynyl radical is two to eighteen carbon atoms (C₂-C₁₈). In other examples, the alkynyl radical is C₂-C₁₂, C₂-C₁₀, C₂-C₈, C₂-C₆ or C₂-C₃. Examples include, but are not limited to, ethynyl (—C≡CH), prop-1-ynyl (—C≡C≡CH₃), prop-2-ynyl (propargyl, —CH₂C≡CH), but-1-ynyl, but-2-ynyl and but-3-ynyl.

“Alkylene” refers to a saturated, branched or straight chain hydrocarbon group having two monovalent radical centers derived by the removal of two hydrogen atoms from the same or two different carbon atoms of a parent alkane. In one example, the divalent alkylene group is one to eighteen carbon atoms (C₁-C₁₈). In other examples, the divalent alkylene group is C₀-C₆, C₀-C₅, C₀-C₃, C₁-C₁₂, C₁-C₁₀, C₁-C₈, C₁-C₆, C₁-C₅, C₁-C₄, or C₁-C₃. The group C₀ alkylene refers to a bond. Example alkylene groups include methylene (—CH₂—), 1,1-ethyl (—CH(CH₃)—), (1,2-ethyl (—CH₂CH₂—), 1,1-propyl (—CH(CH₂CH₃)—), 2,2-propyl (—C(CH₃)₂—), 1,2-propyl (—CH(CH₃)CH₂—), 1,3-propyl (—CH₂CH₂CH₂—), 1,1-dimethyleth-1,2-yl (—C(CH₃)₂CH₂—), 1,4-butyl (—CH₂CH₂CH₂CH₂—), and the like.

“Alkenylene” refers to an unsaturated, branched or straight chain hydrocarbon group having two monovalent radical centers derived by the removal of two hydrogen atoms from the same or two different carbon atoms of a parent alkene. In one example, the alkenylene group is two to eighteen carbon atoms (C₂-C₁₈). In other examples, the alkenylene group is C₂-C₁₂, C₂-C₁₀, C₂-C₈, C₂-C₆ or C₂-C₃. An exemplary alkenylene group is 1,2-ethylene (—CH═CH—).

“Alkynylene” refers to an unsaturated, branched or straight chain hydrocarbon group having two monovalent radical centers derived by the removal of two hydrogen atoms from the same or two different carbon atoms of a parent alkyne. In one example, the alkynylene radical is two to eighteen carbon atoms (C₂-C₁₈). In other examples, the alkynylene radical is C₂-C₁₂, C₂-C₁₀, C₂-C₈, C₂-C₆ or C₂-C₃. Example alkynylene radicals include: acetylene (—C≡C—)₅ propargyl (—CH₂C≡C—)₅ and 4-pentynyl (—CH₂CH₂CH₂C≡C—).

“Amidine” means the group —C(NH)—NHR in which R is hydrogen, alkyl, a cycloalkyl, a heterocyclyl, cycloalkyl-substituted alkyl or heterocyclyl-substituted alkyl wherein the alkyl, alkoxy, cycloalkyl and heterocyclyl are as defined herein. A particular amidine is the group —NH—C(NH)—NH₂.

“Amino” means primary (i.e., —NH₂), secondary (i.e., —NRH) and tertiary (i.e., —NRR) amines, that are optionally substituted, in which R is alkyl, alkoxy, a cycloalkyl, a heterocyclyl, cycloalkyl-substituted alkyl or heterocyclyl-substituted alkyl wherein the alkyl, alkoxy, cycloalkyl and heterocyclyl are as defined herein. Particular secondary and tertiary amines are alkylamine, dialkylamine, arylamine, diarylamine, aralkylamine and diaralkylamine wherein the alkyl is as herein defined and optionally substituted. Particular secondary and tertiary amines are methylamine, ethylamine, propylamine, isopropylamine, phenylamine, benzylamine dimethylamine, diethylamine, dipropylamine and diisopropylamine.

“Amino-protecting group” as used herein refers to a derivative of the groups commonly employed to block or protect an amino group while reactions are carried out on other functional groups on the compound. Examples of such protecting groups include carbamates, amides, alkyl and aryl groups, imines, as well as many N-heteroatom derivatives which can be removed to regenerate the desired amine group. Particular amino protecting groups are Pmb (p-Methoxybenzyl), Boc (tert-Butyloxycarbonyl), Fmoc (9-Fluorenylmethyloxycarbonyl) and Cbz (Carbobenzyloxy). Further examples of these groups are found in T. W. Greene and P. G. M. Wuts, “Protective Groups in Organic Synthesis”, 2^{nd} ed., John Wiley & Sons, Inc., New York, N.Y., 1991, chapter 7; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981. The term “protected amino” refers to an amino group substituted by one of the above amino-protecting groups.

“Aryl” when used alone, or as part of another term, means a carbocyclic aromatic group, whether or not fused to one or more groups, having the number of carbon atoms designated, or if no number is designated, up to 14 carbon atoms. One example includes aryl groups having 6-14 carbon atoms. Another example includes aryl groups having 6-10 carbon atoms. Examples of aryl groups include phenyl, naphthyl, biphenyl, phenanthrenyl, naphthacenyl, 1,2,3,4-tetrahydronaphthalenyl, 1H-indenyl, 2,3-dihydro-1H-indenyl, and the like (see e.g., Lang's Handbook of Chemistry (Dean, J. A., ed) 13^{th} ed. Table 7-2 [1985]). A particular aryl is phenyl. “Substituted phenyl” or “substituted aryl” means a phenyl group or aryl group substituted by one, two, three, four or five, for example 1-2, 1-3 or 1-4 substituents chosen from groups specified herein. In one example, optional substituents on aryl are selected from halogen (F, Cl, Br, I), hydroxy, protected hydroxy, cyano, nitro, alkyl (for example C₁-C₆ alkyl), alkoxy (for example C₁-C₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, alkylsulfonylaminoalkyl, arylsulfonylamino, arylsulfonylaminoalkyl, heterocyclylsulfonylamino, heterocyclylsulfonylaminoalkyl, heterocyclyl, aryl, or other groups specified. One or more methyne (CH) and/or methylene (CH₂) groups in these substituents may in turn be substituted by a similar group as those denoted above. Examples of the term “substituted phenyl” include a mono- or di(halo)phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(isopropyl)phenyl, 4-ethylphenyl, 3-(n-propyl)phenyl and the like; a mono or di(alkoxy)phenyl group, for example, 3,4-dimethoxyphenyl, 3-methoxy-4-benzyloxyphenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such 4-carboxyphenyl, a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; or a mono- or di(N-(methylsulfonylamino))phenyl such as 3-(N-methylsulfonylamino))phenyl. Also, the term “substituted phenyl” represents disubstituted phenyl groups where the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like, as well as trisubstituted phenyl groups where the substituents are different, for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino. Particular substituted phenyl groups include the 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-6-methyl sulfonyl aminophenyl groups. Fused aryl rings may also be substituted by any, for example 1, 2 or 3, of the substituents specified herein in the same manner as substituted alkyl groups.

The Term “Oxo” Refers to ═O or (═O)₂.

The terms “cancer” and “cancerous”, “neoplasm”, and “tumor” refer to or describe the physiological condition in mammals that is typically characterized by unregulated cell growth. A “tumor” comprises one or more cancerous cells. Examples of cancer include carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases.

A “chemotherapeutic agent” is an agent useful in the treatment of a given disorder, for example, cancer or inflammatory disorders. Examples of chemotherapeutic agents include NSAIDs; hormones such as glucocorticoids; corticosteroids such as hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomide, methotrexate (MTX), minocycline, sulfasalazine, cyclophosphamide, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), monoclonal antibodies against B cells such as rituximab (RITUXAN®), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); hormone antagonists, such as tamoxifen, finasteride or LHRH antagonists; radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as fenretinide, retinoic acid, including bexarotene (TARGRETIN®); bisphosphonates such as clodronate (for example, BONEFOS® or OSTAC®), etidronate (DIDROCAL®), NE-58095, zoledronic acidzoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Additional chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonistantagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releasing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgensretinoids such as fluoxymesterone, all transretionic acid and fenretinide; onapristone; anti-progesterones; estrogen receptor down-regulators (ERD5); anti-androgens such as flutamide, nilutamide and bicalutamide.

Additional chemotherapeutic agents include therapeutic antibodies such as alemtuzumab (Campath), bevacizumab (AVASTINO, Genentech); cetuximab (ERBITUX®, Imclone); panitumumab (VECTIBIX®, Amgen), rituximab (RITUXAN®, GenentechBiogen Idec), pertuzumab (OMNITARG®, 2C4, Genentech), trastuzumab (HERCEPTINO, Genentech), tositumomab (Bexxar, Corixa, now GSK), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the antiinterleukin-12 (ABT-874J695, Wyeth Research and Abbott Laboratories) which is a recombinant exclusively human-sequence, full-length IgG₁λ antibody genetically modified to recognize interleukin-12 p40 protein.

Chemotherapeutic agents also include “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX) and reshaped human 225 (H225) (see WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, AbgenixAmgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMDMerck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® GenentechOSI Pharmaceuticals); PD 183805 (CI 1033,2-propenamide, N-[4-[(3-chloro-4-fluorophenyl)amino]-7-[3-(4-morpholinyl)propoxy]-6-quinazolinyl]-dihydrochbride, Pfizer Inc.); ZD1839, gefitinib (IRESSA™) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methylpiperidin-4-yl)-pyrimido[5,4-d]pyrimidine-2,8-di amine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quino linyl]-4-(dimethylamino)-2-butenamide) (Wyeth); AG1478 (Pfizer); AG1571 (SU 5271; Pfizer); dual EGFRHER2 tyrosine kinase inhibitors such as lapatinib (TYKERB®, GSK572016 or N-[3-chloro-4-[(3 fluorophenyl)methoxy]phenyl]-6[5[[[2methyl sulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVECJ, available from Glaxo SmithKline); multi-targeted tyrosine kinase inhibitors such as sunitinib (SUTENT®, available from Pfizer); VEGF receptor tyrosine kinase inhibitors such as vatalanib (PTK787ZK222584, available from NovartisSchering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tyrphostins (U.S. Pat. No. 5,804,396); ZD6474 (Astra Zeneca); PTK-787 (NovartisSchering AG); pan-HER inhibitors such as CI-1033 (Pfizer); Affinitac (ISIS 3521; IsisLilly); imatinib mesylate (GLEEVECT); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (NovartisSchering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include asthma treatment agents, including inhaled corticosteroids such as fluticasone, budesonide, mometasone, flunisolide and beclomethasone; leukotriene modifiers, such as montelukast, zafirlukast and zileuton; long-acting beta agonists, such as salmeterol and formoterol; combinations of the above such as combinations of fluticasone and salmeterol, and combinations of budesonide and formoterol; theophylline; short-acting beta agonists, such as albuterol, levalbuterol and pirbuterol; ipratropium; oral and intravenous corticosteroids, such as prednisone and methylprednisolone; omalizumab; lebrikizumab; antihistamines; and decongestants; cromolyn; and ipratropium.

The term “NSAID” and the terms “non-steroidal anti-inflammatory drug” refer to therapeutic agents with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

Additionally, chemotherapeutic agents include pharmaceutically acceptable salts, acids or derivatives of any of chemotherapeutic agents, described herein, as well as combinations of two or more of them.

“Cycloalkyl” refers to a non-aromatic, saturated or partially unsaturated hydrocarbon ring group wherein the cycloalkyl group may be optionally substituted independently with one or more substituents described herein. In one example, the cycloalkyl group is 3 to 12 carbon atoms (C₃-C₁₂). In other examples, cycloalkyl is C₃-C₈, C₃-C₁₀ or C₅-C₁₀. In other examples, the cycloalkyl group, as a monocycle, is C₃-C₈, C₃-C₆ or C₅-C₆. In another example, the cycloalkyl group, as a bicycle, is C₇-C₁₂. In another example, the cycloalkyl group, as a spiro system, is C₅-C₁₂. Examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[2.2.1]heptane, bicyclo[3.1.1]heptane, bicyclo[2.2.2]octane and bicyclo[3.2.2]nonane. Examples of spiro cycloalkyl include, spiro[2.2]pentane, spiro[2.3]hexane, spiro[2.4]heptane, spiro[2.5]octane and spiro[4.5]decane.

“Carboxy-protecting group” as used herein refers to those groups that are stable to the conditions of subsequent reaction(s) at other positions of the molecule, which may be removed at the appropriate point without disrupting the remainder of the molecule, to give the unprotected carboxy-group. Examples of carboxy protecting groups include ester groups and heterocyclyl groups. Ester derivatives of the carboxylic acid group may be employed to block or protect the carboxylic acid group while reactions are carried out on other functional groups on the compound. Examples of such ester groups include substituted arylalkyl, including substituted benzyls, such as 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, alkyl or substituted alkyl esters such as methyl, ethyl, t-butyl allyl or t-amyl, triphenylmethyl (trityl), 4-methoxytrityl, 4,4′-dimethoxytrityl, 4,4′,4″-trimethoxytrityl, 2-phenylprop-2-yl, thioesters such as t-butyl thioester, silyl esters such as trimethylsilyl, t-butyldimethylsilyl esters, phenacyl, 2,2,2-trichloroethyl, beta-(trimethylsilyl)ethyl, beta-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and like moieties. Another example of carboxy-protecting groups are heterocyclyl groups such as 1,3-oxazolinyl. Further examples of these groups are found in T. W. Greene and P.

G. M. Wuts, “Protective Groups in Organic Synthesis”, 2^{nd} ed., John Wiley & Sons, Inc., New York, N.Y., 1991, chapter 5; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981, Chapter 5. The term “protected carboxy” refers to a carboxy group substituted by one of the above carboxy-protecting groups.

“Guanidine” means the group —NH—C(NH)—NHR in which R is hydrogen, alkyl, alkoxy, a cycloalkyl, a heterocyclyl, cycloalkyl-substituted alkyl or heterocyclyl-substituted alkyl wherein the alkyl, alkoxy, cycloalkyl and heterocyclyl are as defined herein. A particular guanidine is the group —NH—C(NH)—NH₂.

“Hydroxy-protecting group” as used herein refers to a derivative of the hydroxy group commonly employed to block or protect the hydroxy group while reactions are carried out on other functional groups on the compound. Examples of such protecting groups include tetrahydropyranyloxy, benzoyl, acetoxy, carbamoyloxy, benzyl, and silylethers (e.g., TBS, TBDPS) groups. Further examples of these groups are found in T. W. Greene and P. G. M. Wuts, “Protective Groups in Organic Synthesis”, 2^{nd}ed., John Wiley & Sons, Inc., New York, N.Y., 1991, chapters 2-3; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981. The term “protected hydroxy” refers to a hydroxy group substituted by one of the above hydroxy-protecting groups.

“Heterocyclic group”, “heterocyclic”, “heterocycle”, “heterocyclyl”, or “heterocyclo” alone, and when used as a moiety in a complex group such as a heterocycloalkyl group, are used interchangeably and refer to any mono-, bi-, tricyclic or spiro, saturated or unsaturated, aromatic (heteroaryl) or non-aromatic, ring system, having 3 to 20 ring atoms, where the ring atoms are carbon, and at least one atom in the ring or ring system is a heteroatom selected from nitrogen, sulfur or oxygen. In some embodiments, a heterocyclyl is defined as an aromatic ring system (heteroaryl). In some embodiments, a heterocyclyl is defined as a non-aromatic ring system, such as heterocycloalkyl. In one example, heterocyclyl includes 3-12 ring atoms and includes monocycles, bicycles, tricycles and spiro ring systems, wherein the ring atoms are carbon, and at least one atom in the ring or ring system is a heteroatom selected from nitrogen, sulfur or oxygen. In one example, heterocyclyl includes 1 to 4 heteroatoms. In another example, heterocyclyl includes 3- to 7-membered monocycles having one or more heteroatoms selected from nitrogen, sulfur or oxygen. In another example, heterocyclyl includes 4- to 6-membered monocycles having one or more heteroatoms selected from nitrogen, sulfur or oxygen. In another example, heterocyclyl includes 3-membered monocycles. In another example, heterocyclyl includes 4-membered monocycles. In another example, heterocyclyl includes 5-6-membered monocycles. In one example, the heterocyclyl group includes 0 to 3 double bonds. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Example heterocycles are oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocycles containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5 membered ring heterocycles containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Example benzo-fused 5-membered heterocycles are benzoxazol-2-yl, benzthiazol-2-yl and benzimidazol-2-yl. Example 6-membered heterocycles contain one to three nitrogen atoms and optionally a sulfur or oxygen atom, for example morpholinyl, piperidinyl, tetrahydropyranyl, pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are other example heterocycle groups. Substituents for “optionally substituted heterocycles” include, for example, hydroxyl, alkyl, alkoxy, acyl, halogen, mercapto, oxo, carboxyl, halo-substituted alkyl, amino, cyano, nitro, amidino, guanidino. “Heterocyclene” by itself or as part of another substituent means a divalent radical derived from a heterocyclic group.

“Heteroaryl” alone and when used as a moiety in a complex group such as a heteroaralkyl group, refers to any mono-, bi-, or tricyclic ring system where at least one ring is a 5- or 6-membered aromatic ring containing from 1 to 4 heteroatoms selected from nitrogen, oxygen, and sulfur, and in an example embodiment, at least one heteroatom is nitrogen. See, for example, Lang's Handbook of Chemistry, supra. Included in the definition are any bicyclic groups where any of the above heteroaryl rings are fused to an aryl ring. In one embodiment, heteroaryl includes 4-6 membered monocyclic aromatic groups where one or more ring atoms is nitrogen, sulfur or oxygen. In another embodiment, heteroaryl includes 5-6 membered monocyclic aromatic groups where one or more ring atoms is nitrogen, sulfur or oxygen. Example heteroaryl groups (whether substituted or unsubstituted) include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, imidazol[1,2-a]pyrimidinyl and purinyl, as well as benzo-fused derivatives, for example benzoxazolyl, benzofuryl, benzothiazolyl, benzothiadiazolyl, benzotriazolyl, benzoimidazolyl and indolyl. Additional examples of “heteroaryl” groups are: 1,3-thiazol-2-yl, 4-(carboxymethyl)-5-methyl-1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-c arboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl sodium salt, 2,5-dihydro-5-oxo-6-hydroxy-2-methyl-astriazin-3-yl sodium salt, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl and 8-aminotetrazolo[1,5-b]-pyridazin-6-yl. Heteroaryl groups are optionally substituted as described for heterocycles.

In particular embodiments, a heterocyclyl group is attached at a carbon atom of the heterocyclyl group. By way of example, carbon bonded heterocyclyl groups include bonding arrangements at position 2, 3, 4, 5, or 6 of a pyridine ring, position 3, 4, 5, or 6 of a pyridazine, position 2, 4, 5, or 6 of a pyrimidine ring, position 2, 3, 5, or 6 of a pyrazine ring, position 2, 3, 4, or 5 of a furan, tetrahydrofuran, thiofuran, thiophene, pyrrole or tetrahydropyrrole ring, position 2, 4, or 5 of an oxazole, imidazole or thiazole ring, position 3, 4, or 5 of an isoxazole, pyrazole, or isothiazole ring, position 2 or 3 of an aziridine ring, position 2, 3, or 4 of an azetidine ring, position 2, 3, 4, 5, 6, 7, or 8 of a quinoline ring or position 1, 3, 4, 5, 6, 7, or 8 of an isoquinoline ring.

In certain embodiments, the heterocyclyl group is N-attached. By way of example, the nitrogen bonded heterocyclyl or heteroaryl group include bonding arrangements at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

“Leaving group” refers to a portion of a first reactant in a chemical reaction that is displaced from the first reactant in the chemical reaction. Examples of leaving groups include, but are not limited to, halogen atoms, alkoxy and sulfonyloxy groups. Example sulfonyloxy groups include, but are not limited to, alkylsulfonyloxy groups (for example methyl sulfonyloxy (mesylate group) and trifluoromethylsulfonyloxy (triflate group)) and arylsulfonyloxy groups (for example p-toluenesulfonyloxy (tosylate group) and p-nitrosulfonyloxy (nosylate group)).

“Optionally substituted” unless otherwise specified means that a group may be unsubstituted or substituted by one or more (e.g., 0, 1, 2, 3 or 4) of the substituents listed for that group in which said substituents may be the same or different. In an embodiment an optionally substituted group has 1 substituent. In another embodiment an optionally substituted group has 2 substituents. In another embodiment an optionally substituted group has 3 substituents.

Optional substituents for alkyl radicals, such as alkylene, alkenyl, alkynyl, heteroalkyl and cycloalkyl, can be a variety of groups including, but not limited to, halogen, oxo, CN, NO₂, —N₃, OR′, perfluoro-C₁₋₄ alkoxy, unsubstituted cycloalkyl, unsubstituted aryl (e.g., phenyl), unsubstituted heterocyclyl, NR′R″, SR′, SiR′R″R′″, OC(O)R′, C(O)R′, CO₂R′, CONR′R″, OC(O)NR′R″, NR″C(O)R′, NR′″C(O)NR′R″, NR″C(O)₂R′, S(O)₂R′, S(O)₂NR′R″, NR′S(O)₂R″, NR′″S(O)₂NR′R″, amidino, guanidine, (CH₂)₁₋₄OR′, (CH₂)₁₋₄NR′R″, (CH₂)₁₋₄SR′, (CH₂)₁₋₄SiR′R″R″, (CH₂)₁₋₄OC(O)R′, (CH₂)₁₋₄C(O)R′, (CH₂)₁₋₄CO₂R′, and (CH₂)₁₋₄CONR′R″, or combinations thereof, in a number ranging from zero to (2m′+1), where m′ is the total number of carbon atoms in such radical. R′, R″ and R′ each independently refer to groups including, for example, hydrogen; unsubstituted C₁₋₆ alkyl; unsubstituted heteroalkyl; unsubstituted aryl; aryl substituted with 1-3 halogens, unsubstituted C₁₋₆ alkyl, C₁₋₆ alkoxy or C₁₋₆ thioalkoxy groups, unsubstituted aryl-C₁₋₄ alkyl groups, and unsubstituted heteroaryl. When R′ and R″ are attached to the same nitrogen atom, they can be combined with the nitrogen atom to form a 3-, 4-, 5-, 6-, or 7-membered ring wherein a ring atom is optionally substituted with N, O or S. For example, NR′R″ is meant to include 1-pyrrolidinyl and 4-morpholinyl. When a substituent for the alkyl radicals (including those groups often referred to as alkylene, alkenyl, alkynyl, heteroalkyl and cycloalkyl) contains an alkylene linker (e.g., (CH₂)₁₋₄NR′R″), the alkylene linker includes halo variants as well. For example, the linker “(CH₂)₁₋₄” when used as part of a substituent is meant to include difluoromethylene, 1,2-difluoroethylene, etc.

Similarly, optional substituents for the aryl and heterocyclyl groups are varied. In some embodiments, substituents for aryl and heterocyclyl groups are selected from the group including, but not limited to, halogen, OR′, OC(O)R′, NR′R″, SR′, R′, CN, NO₂, CO₂R′, CONR′R″, C(O)R′, OC(O)NR′R″, NR″C(O)R′, NR″C(O)₂R′, NR′C(O)NR″R′″, S(O)R′, S(O)₂R′, S(O)₂NR′R″, NR′S(O)₂R″, N₃, perfluoro-C₁₋₄ alkoxy, perfluoro-C₁₋₄ alkyl, (CH₂)₁₋₄OR′, (CH₂)₁₋₄NR′R″, (CH₂)₁₋₄SR′, (CH₂)₁₋₄SiR′R″R″, (CH₂)₁₋₄OC(O)R′, (CH₂)₁₋₄C(O)R′, (CH₂)₁₋₄CO₂R′, (CH₂)₁₋₄CONR′R″, or combinations thereof, in a number ranging from zero to the total number of open valences on the aromatic ring system; and where R′, R″ and R′ are independently selected from hydrogen, C₁₋₆ alkyl, C₃₋₆ cycloalkyl, C₂₋₆ alkenyl, C₂₋₆ alkynyl, unsubstituted aryl, and unsubstituted heteroaryl. Other suitable substituents include each of the above aryl substituents attached to a ring atom by an alkylene tether of from 1-4 carbon atoms. When a substituent for the aryl or heteroaryl group contains an alkylene linker (e.g., (CH₂)₁₋₄NR′R″), the alkylene linker optionally includes halo variants as well. For example, the linker “(CH₂)₁₋₄” when used as part of a substituent is meant to include difluoromethylene, 1,2-difluoroethylene, etc.

In certain embodiments, divalent groups are described generically without specific bonding configurations, for example in the group —CH₂C(O)—. It is understood that the generic description is meant to include both bonding configurations, unless specified otherwise. For example, in the group R¹—R²—R³, if the group R² is described as —CH₂C(O)—, then it is understood that this group can be bonded both as R¹—CH₂C(O)—R³, and as R¹—C(O)CH₂—R³, unless specified otherwise.

“Package insert” is used to refer to instructions customarily included in commercial packages of therapeutic products that contain information about the indications, usage, dosage, administration, contraindications or warnings concerning the use of such therapeutic products.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, benzenesulfonic acid, p-toluenesulfonic acid, salicyclic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly base addition salts are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particular organic non-toxic bases are isopropylamine, diethylamine, ethanolamine, tromethamine, dicyclohexylamine, choline, and caffeine.

A “sterile” formulation is aseptic or free from all living microorganisms and their spores.

“Stereoisomers” refer to compounds which have identical chemical constitution, but differ with regard to the arrangement of the atoms or groups in space. Stereoisomers include diastereomers, enantiomers, conformers and the like.

“Chiral” refers to molecules which have the property of non-superimposability of the mirror image partner, while the term “achiral” refers to molecules which are superimposable on their mirror image partner.

“Diastereomer” refers to a stereoisomer with two or more centers of chirality and whose molecules are not mirror images of one another. Diastereomers have different physical properties, e.g., melting points, boiling points, spectral properties or biological activities. Mixtures of diastereomers may separate under high resolution analytical procedures such as electrophoresis and chromatography such as HPLC.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined. Unless otherwise specified, if solid wedges or dashed lines are used, relative stereochemistry is intended. If a discrepancy exists between a structure and its name, the name governs.

A “solvate” refers to an association or complex of one or more solvent molecules and a compound of the present invention. Examples of solvents that form solvates include water, isopropanol, ethanol, methanol, DMSO, ethyl acetate, acetic acid, and ethanolamine. The term “hydrate” refers to the complex where the solvent molecule is water.

A “subject,” “individual,” or “patient” is a vertebrate. In certain embodiments, the vertebrate is a mammal. Mammals include, but are not limited to, farm animals (such as cows), sport animals, pets (such as cats, dogs, and horses), primates, mice and rats. In certain embodiments, a mammal is a human.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Therapeutically effective amount” means an amount of a compound of the present invention that (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. In the case of cancer, the therapeutically effective amount of the drug may reduce the number of cancer cells; reduce the tumor size; inhibit (i.e., slow to some extent or stop) cancer cell infiltration into peripheral organs; inhibit (i.e., slow to some extent or stop) tumor metastasis; inhibit, to some extent, tumor growth; or relieve to some extent one or more of the symptoms associated with the cancer. To the extent the drug may prevent growth or kill existing cancer cells, it may be cytostatic and/or cytotoxic. For cancer therapy, efficacy can, for example, be measured by assessing the time to disease progression (TTP) or determining the response rate (RR). In the case of inflammatory or immunological disorders, the therapeutic effective amount is an amount sufficient to decrease or alleviate an allergic disorder, the symptoms of an autoimmune or inflammatory disease, or the symptoms of an acute inflammatory reaction (e.g., asthma). In some embodiments, a therapeutically effective amount is an amount of a chemical entity described herein sufficient to significantly decrease the activity, expression or number of Th2 cytokines or B-cells.

“Treatment” (and variations such as “treat” or “treating”) refers to clinical intervention in an attempt to alter the natural course of the individual or cell being treated, and can be performed either for prophylaxis or during the course of clinical pathology. Desirable effects of treatment include preventing occurrence or recurrence of disease (e.g., asthma), alleviation of symptoms, diminishment of any direct or indirect pathological consequences of the disease, stabilized (i.e., not worsening) state of disease, preventing metastasis, decreasing the rate of disease progression, amelioration or palliation of the disease state, prolonging survival as compared to expected survival if not receiving treatment and remission or improved prognosis. In some embodiments, compounds of the invention are used to delay development of a disease or disorder or to slow the progression of a disease or disorder. Those in need of treatment include those already with the condition or disorder as well as those prone to have the condition or disorder, (for example, through a genetic mutation) or those in which the condition or disorder is to be prevented.

The terms “inhibiting,” “reducing,” or “prevention,” or any variation of these terms, includes any measurable decrease or complete inhibition to achieve a desired result. For example, there may be a decrease of about, at least about, or at most about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity (e.g., ITK kinase activity) compared to normal.

The terms “compound(s) of this invention,” and “compound(s) of the present invention”, unless otherwise indicated, include compounds of formulas (AA), (A), (I), (II), (IIa), (IIb), (III), (IIIa) and (IIIb) and stereoisomers, tautomers, solvates, metabolites, isotopes, salts (e.g., pharmaceutically acceptable salts), and prodrugs thereof.

Any compound or genus of compounds discussed herein may be specifically excluded from any embodiment discussed herein.

The use of the term “or” is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value. In any embodiment discussed in the context of a numerical value used in conjunction with the term “about,” it is specifically contemplated that the term about can be omitted.

Following long-standing patent law, the words “a” and “an,” when used in conjunction with the word “comprising” in the claims or specification, denotes one or more, unless specifically noted.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc., of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible, are specifically contemplated unless specifically indicated to the contrary. Likewise, any subset or combination of these is also specifically contemplated and disclosed. This concept applies to all aspects of this disclosure including, but not limited to, steps in methods using the disclosed compounds and compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed. It is therefore contemplated that any embodiment discussed in this specification can be implemented with respect to any method, compound, kit, or composition, etc., described herein, and vice versa.

Publications cited herein and the material for which they are cited are hereby specifically incorporated by reference in their entireties.

Inhibitors of ITK

Provided herein are compounds of formula (AA):

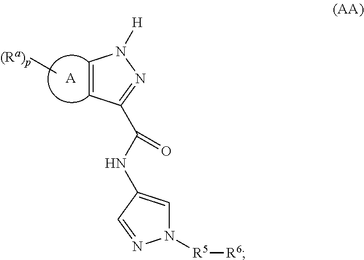

or stereoisomers or a pharmaceutically acceptable salt thereof, wherein:

ring A is a 5-7-membered cycloalkyl or 5-7-membered heterocyclyl;

p is 0, 1, 2, 3, 4, 5, 6, 7 or 8;

each R^{a} is independently a bond, hydrogen, C₁-C₁₂ alkyl, C₂-C₁₂ alkenyl, C₂-C₁₂ alkynyl, C₁-C₆ alkylene, C₂-C₆ alkenylene, C₂-C₆ alkynylene, halogen, —CN, —OR⁷, —SR⁷, —NR⁷R⁸, —CF₃, —CHF₂, —CH₂F, —OCF₃, —NO₂, —C(O)R⁷, —C(O)OR⁷, —C(O)NR⁷R⁸, —NR⁷C(O)R⁸, —S(O)₁₋₂R⁷, —NR⁷S(O)₁₋₂R⁸, —S(O)₁₋₂NR⁷R⁸, C₃-C₆ cycloalkyl, 3-10-membered heterocyclyl or 6-10 membered aryl, wherein each R^{a}, other than a bond and hydrogen, are independently optionally substituted by R⁹, or

two R^{a} are taken together with the atoms to which they are attached to form a C₁-C₆ alkylene, C₂-C₆ alkenylene, C₂-C₆ alkynylene, C₃-C₆ cycloalkyl, 3-10-membered heterocyclyl or 6-10 membered aryl, wherein said cycloalkyl, heterocyclyl and aryl are independently optionally substituted by R⁹, or

two R^{a} are taken together with the atom to which they are attached to form a C₃-C₆ cycloalkyl or 3-10-membered heterocyclyl, wherein said cycloalkyl and heterocyclyl are independently optionally substituted by R⁹;

R⁵ is hydrogen, C₁-C₆ alkylene, C₂-C₆ alkenylene, C₂-C₆ alkynylene, or 3-10-membered heterocyclene wherein said alkylene, alkenylene, alkynylene and heterocyclene are independently optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —CHF₂, —CH₂F, —OCF₃, C₃-C₆ cycloalkyl, 3-10-membered heterocyclyl or 6-10 membered aryl, and wherein said alkyl, alkenyl, alkynyl, cycloalkyl, heterocyclyl and aryl are independently optionally substituted by R²⁰;

R⁶ is hydrogen, C₃-C₁₀ cycloalkyl, 3-10-membered heterocyclyl or 6-10-membered aryl, wherein R⁶ is independently optionally substituted by R⁹, or R₆ is absent when R₅ is hydrogen;

each R⁷ and R⁸ are independently hydrogen, C₁-C₆ alkyl, C₃-C₆ cycloalkyl, 3-6-membered heterocyclyl or phenyl, wherein said alkyl, cycloalkyl, heterocyclyl and phenyl are independently optionally substituted by halogen, —CN, —CF₃, —CHF₂, —CH₂F, —OCF₃ or oxo; or

R⁷ and R⁸ are independently taken together with the atom to which they are attached to form a 3-6 membered heterocyclyl optionally substituted by halogen, oxo or C₁-C₆ alkyl optionally substituted by halogen or oxo;
each R⁹ is independently hydrogen, oxo, C₁-C₁₂ alkyl, C₂-C₁₂ alkenyl, C₂-C₁₂ alkynyl, halogen, —(C₀-C₆ alkylene)CN, —(C₀-C₆ alkylene)OR¹⁰, —(C₀-C₆ alkylene)SR¹⁰, —(C₀-C₆ alkylene)NR¹⁰R¹¹, —(C₀-C₆ alkylene)CF₃, —(C₀-C₆ alkylene)NO₂, —(C₀-C₆ alkylene)C(O)R¹⁰, —(C₀-C₆ alkylene)C(O)OR¹⁰, —(C₀-C₆ alkylene)C(O)NR¹⁰R¹¹, —(C₀-C₆ alkylene)NR¹⁰C(O)R¹¹, —(C₀-C₆ alkylene)S(O)₁₋₂R¹⁰, —(C₀-C₆ alkylene)NR¹⁰S(O)₁₋₂R¹¹, —(C₀-C₆ alkylene)S(O)₁₋₂NR¹⁰R¹¹, —(C₀-C₆ alkylene)(C₃-C₆ cycloalkyl), —(C₀-C₆ alkylene)(3-10-membered heterocyclyl), —(C₀-C₆ alkylene)C(O)(3-10-membered heterocyclyl), or —(C₀-C₆ alkylene)(6-10 membered aryl), wherein each R⁹, other than hydrogen, is independently optionally substituted by halogen, oxo, —CF₃, —CN, —OR¹², —SR¹², —NR¹²R¹³, —C(O)R¹², —S(O)₁₋₂R¹², C₁-C₆ alkyl optionally substituted by oxo or halogen, C₂-C₆ alkenyl optionally substituted by oxo or halogen, or C₂-C₆ alkynyl optionally substituted by oxo or halogen;

each R¹⁰ and R¹¹ are independently hydrogen, C₁-C₆ alkyl, C₂-C₆ alkenyl, C₂-C₆ alkynyl, 3-6-membered heterocyclyl, phenyl or C₃-C₆ cycloalkyl, wherein said alkyl, alkenyl, alkynyl, heterocyclyl, phenyl and cycloalkyl are independently optionally substituted by halogen, oxo, —CF₃, —OCF₃, —OR¹⁴, —SR¹⁴, —NR¹⁴R¹⁵, —CN, 3-6-membered heterocyclyl, phenyl, C₃-C₆ cycloalkyl or C₁-C₆ alkyl optionally substituted by halogen or oxo; or

R¹⁰ and R¹¹ are independently taken together with the atom to which they are attached to form a 3-6 membered heterocyclyl optionally substituted by halogen, oxo or C₁-C₆ alkyl optionally substituted by halogen or oxo;

each R¹² and R¹³ are independently hydrogen or C₁-C₆ alkyl optionally substituted by halogen or oxo; or

R¹² and R¹³ are independently taken together with the atom to which they are attached to form a 3-6 membered heterocyclyl optionally substituted by halogen, oxo or C₁-C₆ alkyl optionally substituted by halogen;

each R¹⁴ and R¹⁵ are independently hydrogen or C₁-C₆ alkyl optionally substituted by halogen or oxo; or

R¹⁴ and R¹⁵ are independently taken together with the atom to which they are attached to form a 3-6 membered heterocyclyl optionally substituted by halogen, oxo or C₁-C₆ alkyl optionally substituted by halogen;

each R¹⁶ and R¹⁷ are independently hydrogen, —S(O)₁₋₂C₁-C₆ alkyl, C₁-C₆ alkyl, C₂-C₆ alkenyl, C₂-C₆ alkynyl, 3-6-membered heterocyclyl, phenyl or C₃-C₆ cycloalkyl, wherein said alkyl, alkenyl, alkynyl, heterocyclyl, phenyl and cycloalkyl are independently optionally substituted by halogen, oxo, —CF₃, —OCF₃, —OR¹⁸, —SR¹⁸, —NR¹⁸R¹⁹, —CN, 3-6-membered heterocyclyl, phenyl, C₃-C₆ cycloalkyl or C₁-C₆ alkyl optionally substituted by halogen or oxo; or

R¹⁶ and R¹⁷ are independently taken together with the atom to which they are attached to form a 3-6 membered heterocyclyl optionally substituted by halogen, oxo or C₁-C₆ alkyl optionally substituted by halogen or oxo;

each R¹⁸ and R¹⁹ are independently hydrogen or C₁-C₆ alkyl optionally substituted by halogen or oxo; or

R¹⁸ and R¹⁹ are independently taken together with the atom to which they are attached to form a 3-6 membered heterocyclyl optionally substituted by halogen, oxo or C₁-C₆ alkyl optionally substituted by halogen;

each R²⁰ is independently hydrogen, oxo, C₁-C₁₂ alkyl, C₂-C₁₂ alkenyl, C₂-C₁₂ alkynyl, halogen, —(C₀-C₆ alkylene)CN, —(C₀-C₆ alkylene)OR²¹, —(C₀-C₆ alkylene)SR²¹, —(C₀-C₆ alkylene)NR²¹R²², —(C₀-C₆ alkylene)CF₃, —(C₀-C₆ alkylene)NO₂, —(C₀-C₆ alkylene)C(O)R²¹, —(C₀-C₆ alkylene)C(O)OR²¹, —(C₀-C₆ alkylene)C(O)NR²¹R²², —(C₀-C₆ alkylene)NR²¹C(O)R²², —(C₀-C₆ alkylene)S(O)₁₋₂R²¹, —(C₀-C₆ alkylene)NR²¹S(O)₁₋₂R²², —(C₀-C₆ alkylene)S(O)₁₋₂NR²¹R²², —(C₀-C₆ alkylene)(C₃-C₆ cycloalkyl), —(C₀-C₆ alkylene)(3-10-membered heterocyclyl), —(C₀-C₆ alkylene)C(O)(3-10-membered heterocyclyl), or —(C₀-C₆ alkylene)(6-10 membered aryl), wherein each R²⁰, other than hydrogen, is independently optionally substituted by halogen, oxo, —CF₃, —CN, —OH or C₁-C₆ alkyl optionally substituted by oxo or halogen; and

each R²¹ and R²² are independently hydrogen, C₁-C₆ alkyl or 3-6 membered heterocyclyl wherein said alkyl or heterocycylyl is optionally substituted by halogen or oxo; or

R²¹ and R²² are independently taken together with the atom to which they are attached to form a 3-6 membered heterocyclyl optionally substituted by halogen, oxo or C₁-C₆ alkyl optionally substituted by halogen.

Another aspect includes a compound of formula (A):

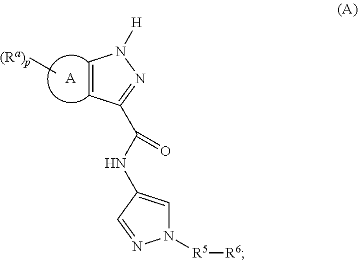

or stereoisomers or a pharmaceutically acceptable salt thereof, wherein:

ring A is a 5-7-membered cycloalkyl or 5-7-membered heterocyclyl;

p is 0, 1, 2, 3, 4, 5, 6, 7 or 8;

each R^{a} is independently a bond, hydrogen, C₁-C₁₂ alkyl, C₂-C₁₂ alkenyl, C₂-C₁₂ alkynyl, C₁-C₆ alkylene, C₂-C₆ alkenylene, C₂-C₆ alkynylene, halogen, —CN, —OR⁷, —SR⁷, —NR⁷R⁸, —CF₃, —CHF₂, —CH₂F, —OCF₃, —NO₂, —C(O)R⁷, —C(O)OR⁷, —C(O)NR⁷R⁸, —NR⁷C(O)R⁸, —S(O)₁₋₂R⁷, —NR⁷S(O)₁₋₂R⁸, —S(O)₁₋₂NR⁷R⁸, C₃-C₆ cycloalkyl, 3-10-membered heterocyclyl or 6-10 membered aryl, wherein each R^{a}, other than a bond and hydrogen, are independently optionally substituted by R⁹, or

two R^{a} are taken together with the atoms to which they are attached to form a C₁-C₆ alkylene, C₂-C₆ alkenylene, C₂-C₆ alkynylene, C₃-C₆ cycloalkyl, 3-10-membered heterocyclyl or 6-10 membered aryl, wherein said cycloalkyl, heterocyclyl and aryl are independently optionally substituted by R⁹, or

two R^{a} are taken together with the atom to which they are attached to form a C₃-C₆ cycloalkyl or 3-10-membered heterocyclyl, wherein said cycloalkyl and heterocyclyl are independently optionally substituted by R⁹;

R⁵ is C₁-C₆ alkylene, C₂-C₆ alkenylene, C₂-C₆ alkynylene, or 3-10-membered heterocyclyl wherein said alkylene, alkenylene and alkynylene are independently optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —CHF₂, —CH₂F, —OCF₃, C₃-C₆ cycloalkyl, 3-10-membered heterocyclyl or 6-10 membered aryl, and wherein said alkyl, alkenyl, alkynyl, cycloalkyl, heterocyclyl and aryl are independently optionally substituted by R²⁰;

R⁶ is hydrogen, C₃-C₁₀ cycloalkyl, 3-10-membered heterocyclyl or 6-10-membered aryl, wherein R⁶ is independently optionally substituted by R⁹;

each R⁷ and R⁸ are independently hydrogen, C₁-C₆ alkyl, C₃-C₆ cycloalkyl, 3-6-membered heterocyclyl or phenyl, wherein said alkyl, cycloalkyl, heterocyclyl and phenyl are independently optionally substituted by halogen, —CN, —CF₃, —CHF₂, —CH₂F, —OCF₃ or oxo; or

R⁷ and R⁸ are independently taken together with the atom to which they are attached to form a 3-6 membered heterocyclyl optionally substituted by halogen, oxo or C₁-C₆ alkyl optionally substituted by halogen or oxo;

each R⁹ is independently hydrogen, oxo, C₁-C₁₂ alkyl, C₂-C₁₂ alkenyl, C₂-C₁₂ alkynyl, halogen, —(C₀-C₆ alkylene)CN, —(C₀-C₆ alkylene)OR¹⁰, —(C₀-C₆ alkylene)SR¹⁰, —(C₀-C₆ alkylene)NR¹⁰R¹¹, —(C₀-C₆ alkylene)CF₃, —(C₀-C₆ alkylene)NO₂, —(C₀-C₆ alkylene)C(O)R¹⁰, —(C₀-C₆ alkylene)C(O)OR¹⁰, —(C₀-C₆ alkylene)C(O)NR¹⁰R¹¹, —(C₀-C₆ alkylene)NR¹⁰C(O)R¹¹, —(C₀-C₆ alkylene)S(O)₁₋₂R¹⁰, —(C₀-C₆ alkylene)NR¹⁰S(O)₁₋₂R¹¹, —(C₀-C₆ alkylene)S(O)₁₋₂NR¹⁰R¹¹, —(C₀-C₆ alkylene)(C₃-C₆ cycloalkyl), —(C₀-C₆ alkylene)(3-10-membered heterocyclyl), —(C₀-C₆ alkylene)C(O)(3-10-membered heterocyclyl), or —(C₀-C₆ alkylene)(6-10 membered aryl), wherein each R⁹, other than hydrogen, is independently optionally substituted by halogen, oxo, —CF₃, —CN, —OR¹², —SR¹², —NR²R³, —C(O)R¹², —S(O)₁₋₂R¹², C₁-C₆ alkyl optionally substituted by oxo or halogen, C₂-C₆ alkenyl optionally substituted by oxo or halogen, or C₂-C₆ alkynyl optionally substituted by oxo or halogen;

each R¹⁰ and R¹¹ are independently hydrogen, C₁-C₆ alkyl, C₂-C₆ alkenyl, C₂-C₆ alkynyl, 3-6-membered heterocyclyl, phenyl or C₃-C₆ cycloalkyl, wherein said alkyl, alkenyl, alkynyl, heterocyclyl, phenyl and cycloalkyl are independently optionally substituted by halogen, oxo, —CF₃, —OCF₃, —OR¹⁴, —SR¹⁴, —NR¹⁴R¹⁵, —CN, 3-6-membered heterocyclyl, phenyl, C₃-C₆ cycloalkyl or C₁-C₆ alkyl optionally substituted by halogen or oxo; or

R¹⁰ and R¹¹ are independently taken together with the atom to which they are attached to form a 3-6 membered heterocyclyl optionally substituted by halogen, oxo or C₁-C₆ alkyl optionally substituted by halogen or oxo;

each R¹² and R¹³ are independently hydrogen or C₁-C₆ alkyl optionally substituted by halogen or oxo; or

R¹² and R¹³ are independently taken together with the atom to which they are attached to form a 3-6 membered heterocyclyl optionally substituted by halogen, oxo or C₁-C₆ alkyl optionally substituted by halogen;

each R¹⁴ and R¹⁵ are independently hydrogen or C₁-C₆ alkyl optionally substituted by halogen or oxo; or

R¹⁴ and R¹⁵ are independently taken together with the atom to which they are attached to form a 3-6 membered heterocyclyl optionally substituted by halogen, oxo or C₁-C₆ alkyl optionally substituted by halogen;

each R¹⁶ and R¹⁷ are independently hydrogen, —S(O)₁₋₂C₁-C₆ alkyl, C₁-C₆ alkyl, C₂-C₆ alkenyl, C₂-C₆ alkynyl, 3-6-membered heterocyclyl, phenyl or C₃-C₆ cycloalkyl, wherein said alkyl, alkenyl, alkynyl, heterocyclyl, phenyl and cycloalkyl are independently optionally substituted by halogen, oxo, —CF₃, —OCF₃, —OR¹⁸, —SR¹⁸, —NR¹⁸R¹⁹, —CN, 3-6-membered heterocyclyl, phenyl, C₃-C₆ cycloalkyl or C₁-C₆ alkyl optionally substituted by halogen or oxo; or

R¹⁶ and R¹⁷ are independently taken together with the atom to which they are attached to form a 3-6 membered heterocyclyl optionally substituted by halogen, oxo or C₁-C₆ alkyl optionally substituted by halogen or oxo;

each R¹⁸ and R¹⁹ are independently hydrogen or C₁-C₆ alkyl optionally substituted by halogen or oxo; or

R¹⁸ and R¹⁹ are independently taken together with the atom to which they are attached to form a 3-6 membered heterocyclyl optionally substituted by halogen, oxo or C₁-C₆ alkyl optionally substituted by halogen;

each R²⁰ is independently hydrogen, oxo, C₁-C₁₂ alkyl, C₂-C₁₂ alkenyl, C₂-C₁₂ alkynyl, halogen, —(C₀-C₆ alkylene)CN, —(C₀-C₆ alkylene)OR²¹, —(C₀-C₆ alkylene)SR²¹, —(C₀-C₆ alkylene)NR²¹R²², —(C₀-C₆ alkylene)CF₃, —(C₀-C₆ alkylene)NO₂, —(C₀-C₆ alkylene)C(O)R²¹, —(C₀-C₆ alkylene)C(O)OR²¹, —(C₀-C₆ alkylene)C(O)NR²¹R²², —(C₀-C₆ alkylene)NR²¹C(O)R²², —(C₀-C₆ alkylene)S(O)₁₋₂R²¹, —(C₀-C₆ alkylene)NR²¹S(O)₁₋₂R²², —(C₀-C₆ alkylene)S(O)₁₋₂NR²¹R²², —(C₀-C₆ alkylene)(C₃-C₆ cycloalkyl), —(C₀-C₆ alkylene)(3-10-membered heterocyclyl), —(C₀-C₆ alkylene)C(O)(3-10-membered heterocyclyl), or —(C₀-C₆ alkylene)(6-10 membered aryl), wherein each R²⁰, other than hydrogen, is independently optionally substituted by halogen, oxo, —CF₃, —CN, —OH or C₁-C₆ alkyl optionally substituted by oxo or halogen; and

each R²¹ and R²² are independently hydrogen, C₁-C₆ alkyl, or 3-6 membered heterocyclyl optionally substituted by halogen or oxo, where the 3-6 membered heterocyclyl is optionally omitted; or R²¹ and R²² are independently taken together with the atom to which they are attached to form a 3-6 membered heterocyclyl optionally substituted by halogen, oxo or C₁-C₆ alkyl optionally substituted by halogen.

Another aspect includes a compound of formula (I):

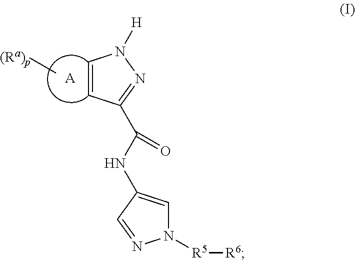

or stereoisomers or a pharmaceutically acceptable salt thereof, wherein:

ring A is a 5-7-membered cycloalkyl or 5-7-membered heterocyclyl;

p is 0, 1, 2, 3, 4, 5, 6, 7 or 8;

each R^{a} is independently a bond, hydrogen, C₁-C₁₂ alkyl, C₂-C₁₂ alkenyl, C₂-C₁₂ alkynyl, C₁-C₆ alkylene, C₂-C₆ alkenylene, C₂-C₆ alkynylene, halogen, —CN, —OR⁷, —SR⁷, —NR⁷R⁸, —CF₃, —CHF₂, —CH₂F, —OCF₃, —NO₂, —C(O)R⁷, —C(O)OR⁷, —C(O)NR⁷R⁸, —NR⁷C(O)R⁸, —S(O)₁₋₂R⁷, —NR⁷S(O)₁₋₂R⁸, —S(O)₁₋₂NR⁷R⁸, C₃-C₆ cycloalkyl, 3-10-membered heterocyclyl or 6-10 membered aryl, wherein each R^{a}, other than a bond and hydrogen, are independently optionally substituted by R⁹, or

two R^{a} are taken together with the atoms to which they are attached to form a C₁-C₆ alkylene, C₂-C₆ alkenylene, C₂-C₆ alkynylene, C₃-C₆ cycloalkyl, 3-10-membered heterocyclyl or 6-10 membered aryl, wherein said cycloalkyl, heterocyclyl and aryl are independently optionally substituted by R⁹, or

two R^{a} are taken together with the atom to which they are attached to form a C₃-C₆ cycloalkyl or 3-10-membered heterocyclyl, wherein said cycloalkyl and heterocyclyl are independently optionally substituted by R⁹;

R⁵ is C₁-C₆ alkylene, C₂-C₆ alkenylene, C₂-C₆ alkynylene, wherein said alkylene, alkenylene and alkynylene are independently optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —CHF₂, —CH₂F, —OCF₃, C₃-C₆ cycloalkyl, 3-10-membered heterocyclyl or 6-10 membered aryl, and wherein said alkyl, alkenyl, alkynyl, cycloalkyl, heterocyclyl and aryl are independently optionally substituted by R²⁰;

R⁶ is hydrogen, C₃-C₁₀ cycloalkyl, 3-10-membered heterocyclyl or 6-10-membered aryl, wherein R⁶ is independently optionally substituted by R⁹;

each R⁷ and R⁸ are independently hydrogen, C₁-C₆ alkyl, C₃-C₆ cycloalkyl, 3-6-membered heterocyclyl or phenyl, wherein said alkyl, cycloalkyl, heterocyclyl and phenyl are independently optionally substituted by halogen, —CN, —CF₃, —CHF₂, —CH₂F, —OCF₃ or oxo; or

R⁷ and R⁸ are independently taken together with the atom to which they are attached to form a 3-6 membered heterocyclyl optionally substituted by halogen, oxo or C₁-C₆ alkyl optionally substituted by halogen or oxo;

each R⁹ is independently hydrogen, oxo, C₁-C₁₂ alkyl, C₂-C₁₂ alkenyl, C₂-C₁₂ alkynyl, halogen, —(C₀-C₆ alkylene)CN, —(C₀-C₆ alkylene)OR¹⁰, —(C₀-C₆ alkylene)SR¹⁰, —(C₀-C₆ alkylene)NR¹⁰R¹¹, —(C₀-C₆ alkylene)CF₃, —(C₀-C₆ alkylene)NO₂, —(C₀-C₆ alkylene)C(O)R¹⁰, —(C₀-C₆ alkylene)C(O)OR¹⁰, —(C₀-C₆ alkylene)C(O)NR¹⁰R¹¹, —(C₀-C₆ alkylene)NR¹⁰C(O)R¹¹, —(C₀-C₆ alkylene)S(O)₁₋₂R¹⁰, —(C₀-C₆ alkylene)NR¹⁰S(O)₁₋₂R¹¹, —(C₀-C₆ alkylene)S(O)₁₋₂NR¹⁰R¹¹, —(C₀-C₆ alkylene)(C₃-C₆ cycloalkyl), —(C₀-C₆ alkylene)(3-10-membered heterocyclyl), —(C₀-C₆ alkylene)C(O)(3-10-membered heterocyclyl), or —(C₀-C₆ alkylene)(6-10 membered aryl), wherein each R⁹, other than hydrogen, is independently optionally substituted by halogen, oxo, —CF₃, —CN, —OR¹², —SR¹², —NR¹²R¹³, —C(O)R¹², —S(O)₁₋₂R¹², C₁-C₆ alkyl optionally substituted by oxo or halogen, C₂-C₆ alkenyl optionally substituted by oxo or halogen, or C₂-C₆ alkynyl optionally substituted by oxo or halogen;

each R¹⁰ and R¹¹ are independently hydrogen, C₁-C₆ alkyl, C₂-C₆ alkenyl, C₂-C₆ alkynyl, 3-6-membered heterocyclyl, phenyl or C₃-C₆ cycloalkyl, wherein said alkyl, alkenyl, alkynyl, heterocyclyl, phenyl and cycloalkyl are independently optionally substituted by halogen, oxo, —CF₃, —OCF₃, —OR¹⁴, —SR¹⁴, —NR¹⁴R¹⁵, —CN, 3-6-membered heterocyclyl, phenyl, C₃-C₆ cycloalkyl or C₁-C₆ alkyl optionally substituted by halogen or oxo; or

R¹⁰ and R¹¹ are independently taken together with the atom to which they are attached to form a 3-6 membered heterocyclyl optionally substituted by halogen, oxo or C₁-C₆ alkyl optionally substituted by halogen or oxo;

each R¹² and R¹³ are independently hydrogen or C₁-C₆ alkyl optionally substituted by halogen or oxo; or

R¹² and R¹³ are independently taken together with the atom to which they are attached to form a 3-6 membered heterocyclyl optionally substituted by halogen, oxo or C₁-C₆ alkyl optionally substituted by halogen;

each R¹⁴ and R¹⁵ are independently hydrogen or C₁-C₆ alkyl optionally substituted by halogen or oxo; or

R¹⁴ and R¹⁵ are independently taken together with the atom to which they are attached to form a 3-6 membered heterocyclyl optionally substituted by halogen, oxo or C₁-C₆ alkyl optionally substituted by halogen;

each R¹⁶ and R¹⁷ are independently hydrogen, —S(O)₁₋₂C₁-C₆ alkyl, C₁-C₆ alkyl, C₂-C₆ alkenyl, C₂-C₆ alkynyl, 3-6-membered heterocyclyl, phenyl or C₃-C₆ cycloalkyl, wherein said alkyl, alkenyl, alkynyl, heterocyclyl, phenyl and cycloalkyl are independently optionally substituted by halogen, oxo, —CF₃, —OCF₃, —OR⁸, —SR⁸, —NR¹⁸R¹⁹, —CN, 3-6-membered heterocyclyl, phenyl, C₃-C₆ cycloalkyl or C₁-C₆ alkyl optionally substituted by halogen or oxo; or

R¹⁶ and R¹⁷ are independently taken together with the atom to which they are attached to form a 3-6 membered heterocyclyl optionally substituted by halogen, oxo or C₁-C₆ alkyl optionally substituted by halogen or oxo;

each R¹⁸ and R¹⁹ are independently hydrogen or C₁-C₆ alkyl optionally substituted by halogen or oxo; or

R¹⁸ and R¹⁹ are independently taken together with the atom to which they are attached to form a 3-6 membered heterocyclyl optionally substituted by halogen, oxo or C₁-C₆ alkyl optionally substituted by halogen;

each R²⁰ is independently hydrogen, oxo, C₁-C₁₂ alkyl, C₂-C₁₂ alkenyl, C₂-C₁₂ alkynyl, halogen, —(C₀-C₆ alkylene)CN, —(C₀-C₆ alkylene)OR²¹, —(C₀-C₆ alkylene)SR²¹, —(C₀-C₆ alkylene)NR²¹R²², —(C₀-C₆ alkylene)CF₃, —(C₀-C₆ alkylene)NO₂, —(C₀-C₆ alkylene)C(O)R²¹, —(C₀-C₆ alkylene)C(O)OR²¹, —(C₀-C₆ alkylene)C(O)NR²¹R²², —(C₀-C₆ alkylene)NR²¹C(O)R²², —(C₀-C₆ alkylene)S(O)₁₋₂R²¹, —(C₀-C₆ alkylene)NR²¹S(O)₁₋₂R²², —(C₀-C₆ alkylene)S(O)₁₋₂NR²¹R²², —(C₀-C₆ alkylene)(C₃-C₆ cycloalkyl), —(C₀-C₆ alkylene)(3-10-membered heterocyclyl), —(C₀-C₆ alkylene)C(O)(3-10-membered heterocyclyl), or —(C₀-C₆ alkylene)(6-10 membered aryl), wherein each R²⁰, other than hydrogen, is independently optionally substituted by halogen, oxo, —CF₃, —CN, —OH or C₁-C₆ alkyl optionally substituted by oxo or halogen; and

each R²¹ and R²² are independently hydrogen or C₁-C₆ alkyl optionally substituted by halogen or oxo; or

R²¹ and R²² are independently taken together with the atom to which they are attached to form a 3-6 membered heterocyclyl optionally substituted by halogen, oxo or C₁-C₆ alkyl optionally substituted by halogen.

Another aspect includes a compound of formula (II):

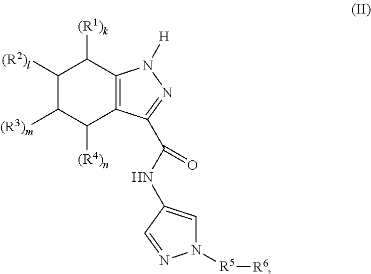

or stereoisomers or a pharmaceutically acceptable salt thereof, wherein:

k, l, m and n are independently 0, 1 or 2;

each R¹, R², R³ and R⁴ are independently a bond, hydrogen, C₁-C₁₂ alkyl, C₂-C₁₂ alkenyl, C₂-C₁₂ alkynyl, C₁-C₆ alkylene, C₂-C₆ alkenylene, C₂-C₆ alkynylene, halogen, —CN, —OR⁷, —SR⁷, —NR⁷R⁸, —CF₃, —CHF₂, —CH₂F, —OCF₃, —NO₂, —C(O)R⁷, —C(O)OR⁷, —C(O)NR⁷R⁸, —NR⁷C(O)R⁸, —S(O)₁₋₂R⁷, —NR⁷S(O)₁₋₂R⁸, —S(O)₁₋₂NR⁷R⁸, C₃-C₆ cycloalkyl, 3-10-membered heterocyclyl or 6-10 membered aryl, wherein each R¹, R², R³ and R⁴, other than a bond and hydrogen, are independently optionally substituted by R⁹, or

one R¹ and one of R², R³ and R⁴ are taken together with the atoms to which they are attached to form a C₁-C₆ alkylene, C₂-C₆ alkenylene, C₂-C₆ alkynylene, C₃-C₆ cycloalkyl, 3-10-membered heterocyclyl or 6-10 membered aryl, wherein said cycloalkyl, heterocyclyl and aryl are independently optionally substituted by R⁹, or

one R² and one of R¹, R³ and R⁴ are taken together with the atoms to which they are attached to form a C₁-C₆ alkylene, C₂-C₆ alkenylene, C₂-C₆ alkynylene, C₃-C₆ cycloalkyl, 3-10-membered heterocyclyl or 6-10 membered aryl, wherein said cycloalkyl, heterocyclyl and aryl are independently optionally substituted by R⁹, or

one R³ and one of R¹, R² and R⁴ are taken together with the atoms to which they are attached to form a C₁-C₆ alkylene, C₂-C₆ alkenylene, C₂-C₆ alkynylene, C₃-C₆ cycloalkyl, 3-10-membered heterocyclyl or 6-10 membered aryl, wherein said cycloalkyl, heterocyclyl and aryl are independently optionally substituted by R⁹, or

one R⁴ and one of R¹, R² and R³ are taken together with the atoms to which they are attached to form a C₁-C₆ alkylene, C₂-C₆ alkenylene, C₂-C₆ alkynylene, C₃-C₆ cycloalkyl, 3-10-membered heterocyclyl or 6-10 membered aryl, wherein said cycloalkyl, heterocyclyl and aryl are independently optionally substituted by R⁹, or

two R¹ are taken together with the atom to which they are attached to form a C₃-C₆ cycloalkyl or 3-10-membered heterocyclyl, wherein said cycloalkyl and heterocyclyl are independently optionally substituted by R⁹, or

two R² are taken together with the atom to which they are attached to form a C₃-C₆ cycloalkyl or 3-10-membered heterocyclyl, wherein said cycloalkyl and heterocyclyl are independently optionally substituted by R⁹, or

two R³ are taken together with the atom to which they are attached to form a C₃-C₆ cycloalkyl or 3-10-membered heterocyclyl, wherein said cycloalkyl and heterocyclyl are independently optionally substituted by R⁹, or

two R⁴ are taken together with the atom to which they are attached to form a C₃-C₆ cycloalkyl or 3-10-membered heterocyclyl, wherein said cycloalkyl and heterocyclyl are independently optionally substituted by R⁹;

R⁵ is C₁-C₆ alkylene, C₂-C₆ alkenylene, C₂-C₆ alkynylene, wherein said alkylene, alkenylene and alkynylene are independently optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —OCF₃, C₃-C₆ cycloalkyl, 3-10-membered heterocyclyl or 6-10 membered aryl, and wherein said alkyl, alkenyl, alkynyl, cycloalkyl, heterocyclyl and aryl are independently optionally substituted by R²⁰;

R⁶ is hydrogen, C₃-C₁₀ cycloalkyl, 3-10-membered heterocyclyl or 6-10-membered aryl, wherein R⁶ is independently optionally substituted by R⁹;

each R⁷ and R⁸ are independently hydrogen, C₁-C₆ alkyl, C₃-C₆ cycloalkyl, 3-6-membered heterocyclyl or phenyl, wherein said alkyl, cycloalkyl, heterocyclyl and phenyl are independently optionally substituted by halogen, —CN, —CF₃, —OCF₃ or oxo; or

R⁷ and R⁸ are independently taken together with the atom to which they are attached to form a 3-6 membered heterocyclyl optionally substituted by halogen, oxo or C₁-C₆ alkyl optionally substituted by halogen or oxo;

each R⁹ is independently hydrogen, oxo, C₁-C₁₂ alkyl, C₂-C₁₂ alkenyl, C₂-C₁₂ alkynyl, halogen, —(C₀-C₆ alkylene)CN, —(C₀-C₆ alkylene)OR¹⁰, —(C₀-C₆ alkylene)SR¹⁰, —(C₀-C₆ alkylene)NR¹⁰R¹¹, —(C₀-C₆ alkylene)CF₃, —(C₀-C₆ alkylene)NO₂, —(C₀-C₆ alkylene)C(O)R¹⁰, —(C₀-C₆ alkylene)C(O)OR¹⁰, —(C₀-C₆ alkylene)C(O)NR¹⁰R¹¹, —(C₀-C₆ alkylene)NR¹⁰C(O)R¹¹, —(C₀-C₆ alkylene)S(O)₁₋₂R¹⁰, —(C₀-C₆ alkylene)NR¹⁰S(O)₁₋₂R¹¹, —(C₀-C₆ alkylene)S(O)₁₋₂NR¹⁰R¹¹, —(C₀-C₆ alkylene)(C₃-C₆ cycloalkyl), —(C₀-C₆ alkylene)(3-10-membered heterocyclyl), —(C₀-C₆ alkylene)C(O)(3-10-membered heterocyclyl), or —(C₀-C₆ alkylene)(6-10 membered aryl), wherein each R⁹, other than hydrogen, is independently optionally substituted by halogen, oxo, —CF₃, —CN, —OR¹², —SR¹², —NR¹²R¹³, —C(O)R¹², —S(O)₁₋₂R¹², C₁-C₆ alkyl optionally substituted by oxo or halogen, C₂-C₆ alkenyl optionally substituted by oxo or halogen, or C₂-C₆ alkynyl optionally substituted by oxo or halogen;

each R¹⁰ and R¹¹ are independently hydrogen, C₁-C₆ alkyl, C₂-C₆ alkenyl, C₂-C₆ alkynyl, 3-6-membered heterocyclyl, phenyl or C₃-C₆ cycloalkyl, wherein said alkyl, alkenyl, alkynyl, heterocyclyl, phenyl and cycloalkyl are independently optionally substituted by halogen, oxo, —CF₃, —OCF₃, —OR¹⁴, —SR¹⁴, —NR¹⁴R¹⁵, —CN, 3-6-membered heterocyclyl, phenyl, C₃-C₆ cycloalkyl or C₁-C₆ alkyl optionally substituted by halogen or oxo; or

R¹⁰ and R¹¹ are independently taken together with the atom to which they are attached to form a 3-6 membered heterocyclyl optionally substituted by halogen, oxo or C₁-C₆ alkyl optionally substituted by halogen or oxo;

each R¹² and R¹³ are independently hydrogen or C₁-C₆ alkyl optionally substituted by halogen or oxo; or

R¹² and R¹³ are independently taken together with the atom to which they are attached to form a 3-6 membered heterocyclyl optionally substituted by halogen, oxo or C₁-C₆ alkyl optionally substituted by halogen;

each R¹⁴ and R¹⁵ are independently hydrogen or C₁-C₆ alkyl optionally substituted by halogen or oxo; or

R¹⁴ and R¹⁵ are independently taken together with the atom to which they are attached to form a 3-6 membered heterocyclyl optionally substituted by halogen, oxo or C₁-C₆ alkyl optionally substituted by halogen;

each R¹⁶ and R¹⁷ are independently hydrogen, —S(O)₁₋₂C₁-C₆ alkyl, C₁-C₆ alkyl, C₂-C₆ alkenyl, C₂-C₆ alkynyl, 3-6-membered heterocyclyl, phenyl or C₃-C₆ cycloalkyl, wherein said alkyl, alkenyl, alkynyl, heterocyclyl, phenyl and cycloalkyl are independently optionally substituted by halogen, oxo, —CF₃, —OCF₃, —OR¹⁸, —SR¹⁸, —NR¹⁸R¹⁹, —CN, 3-6-membered heterocyclyl, phenyl, C₃-C₆ cycloalkyl or C₁-C₆ alkyl optionally substituted by halogen or oxo; or

R¹⁶ and R¹⁷ are independently taken together with the atom to which they are attached to form a 3-6 membered heterocyclyl optionally substituted by halogen, oxo or C₁-C₆ alkyl optionally substituted by halogen or oxo;

each R¹⁸ and R¹⁹ are independently hydrogen or C₁-C₆ alkyl optionally substituted by halogen or oxo; or

R¹⁸ and R¹⁹ are independently taken together with the atom to which they are attached to form a 3-6 membered heterocyclyl optionally substituted by halogen, oxo or C₁-C₆ alkyl optionally substituted by halogen;

each R²⁰ is independently hydrogen, oxo, C₁-C₁₂ alkyl, C₂-C₁₂ alkenyl, C₂-C₁₂ alkynyl, halogen, —(C₀-C₆ alkylene)CN, —(C₀-C₆ alkylene)OR²¹, —(C₀-C₆ alkylene)SR²¹, —(C₀-C₆ alkylene)NR²¹R²², —(C₀-C₆ alkylene)CF₃, —(C₀-C₆ alkylene)NO₂, —(C₀-C₆ alkylene)C(O)R²¹, —(C₀-C₆ alkylene)C(O)OR²¹, —(C₀-C₆ alkylene)C(O)NR²¹R²², —(C₀-C₆ alkylene)NR²¹C(O)R²², —(C₀-C₆ alkylene)S(O)₁₋₂R²¹, —(C₀-C₆ alkylene)NR²¹S(O)₁₋₂R²², —(C₀-C₆ alkylene)S(O)₁₋₂NR²¹R²², —(C₀-C₆ alkylene)(C₃-C₆ cycloalkyl), —(C₀-C₆ alkylene)(3-10-membered heterocyclyl), —(C₀-C₆ alkylene)C(O)(3-10-membered heterocyclyl), or —(C₀-C₆ alkylene)(6-10 membered aryl), wherein each R²⁰, other than hydrogen, is independently optionally substituted by halogen, oxo, —CF₃, —CN, —OH or C₁-C₆ alkyl optionally substituted by oxo or halogen; and

each R²¹ and R²² are independently hydrogen or C₁-C₆ alkyl optionally substituted by halogen or oxo; or

R²¹ and R²² are independently taken together with the atom to which they are attached to form a 3-6 membered heterocyclyl optionally substituted by halogen, oxo or C₁-C₆ alkyl optionally substituted by halogen.

In some embodiments, a compound of formula (AA), (A), (I) or (II) is defined as a compound of formula (IIa) or formula (IIb):

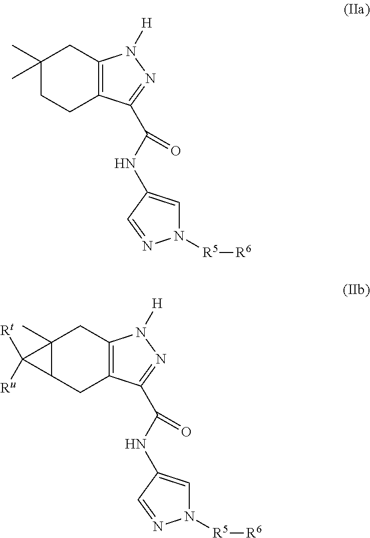

or stereoisomers or a pharmaceutically acceptable salt, wherein:

R^{u} is hydrogen or halogen (e.g., fluoro);

R^{t} is hydrogen or halogen (e.g., fluoro); and

R⁵ and R⁶ are as defined herein.

In certain embodiments, ring A is a 5-membered cycloalkyl. In certain embodiments, ring A is a 6-membered cycloalkyl. In certain embodiments, ring A is a 7-membered cycloalkyl. In certain embodiments, ring A is a 5-membered heterocyclyl. In certain embodiments, ring A is a 6-membered heterocyclyl. In certain embodiments, ring A is a 7-membered heterocyclyl.

In certain embodiments, k, l, m and n are independently 0.

In certain embodiments, k is 1 and l, m and n are 0. In certain embodiments, k is 2 and l, m and n are 0.

In certain embodiments, l is 1 and k, m and n are 0. In certain embodiments, l is 2 and k, m and n are 0.

In certain embodiments, m is 1 and k, l and n are 0. In certain embodiments, m is 2 and k, l and n are 0.

In certain embodiments, n is 1 and k, l and m are 0. In certain embodiments, n is 2 and k, l and m are 0.

In certain embodiments, k, l, m and n are independently 1.

In certain embodiments, k, l, m and n are independently 2.

In certain embodiments, each R¹, R², R³ and R⁴ are independently a bond, hydrogen, C₁-C₁₂ alkyl, C₁-C₆ alkylene, halogen, —OR⁷, C₃-C₆ cycloalkyl or 3-10-membered heterocyclyl, wherein each R¹, R², R³ and R⁴, other than a bond and hydrogen, are independently optionally substituted by R⁹, or

one R¹ and one R⁴ are taken together with the atoms to which they are attached to form a C₁-C₆ alkylene, independently optionally substituted by R⁹, or

one R¹ and one R³ are taken together with the atoms to which they are attached to form a C₁-C₆ alkylene, independently optionally substituted by R⁹, or

one R² and one R⁴ are taken together with the atoms to which they are attached to form a C₁-C₆ alkylene, independently optionally substituted by R⁹, or

one R¹ and one R² are taken together with the atoms to which they are attached to form a C₃-C₆ cycloalkyl independently optionally substituted by R⁹, or

one R² and one R³ are taken together with the atoms to which they are attached to form a C₃-C₆ cycloalkyl independently optionally substituted by R⁹, or

one R³ and one R⁴ are taken together with the atoms to which they are attached to form a C₃-C₆ cycloalkyl independently optionally substituted by R⁹, or

two R² are taken together with the atom to which they are attached to form a C₃-C₆ cycloalkyl or 3-10-membered heterocyclyl, wherein said cycloalkyl and heterocyclyl are independently optionally substituted by R⁹, or

two R³ are taken together with the atom to which they are attached to form a C₃-C₆ cycloalkyl or 3-10-membered heterocyclyl, wherein said cycloalkyl and heterocyclyl are independently optionally substituted by R⁹.

In certain embodiments, each R¹, R², R³ and R⁴ are independently a bond, hydrogen, methyl, ethyl, methylene, ethylene, fluoro, —OH, —OCH₃, —CH₂OH, cyclopropyl, pyrazolo, pyrimidinyl, oxetanyl or tetrahydrofuranyl, wherein each R¹, R², R³ and R⁴, other than a bond and hydrogen, are independently optionally substituted by R⁹, or

one R¹ and one R⁴ are taken together with the atoms to which they are attached to form a methylene or ethylene, independently optionally substituted by R⁹, or

one R¹ and one R³ are taken together with the atoms to which they are attached to form a methylene, independently optionally substituted by R⁹, or

one R² and one R⁴ are taken together with the atoms to which they are attached to form a ethylene, independently optionally substituted by R⁹, or

one R¹ and one R² are taken together with the atoms to which they are attached to form a C₃ cycloalkyl independently optionally substituted by R⁹, or

one R² and one R³ are taken together with the atoms to which they are attached to form a C₃ cycloalkyl independently optionally substituted by R⁹, or

one R³ and one R⁴ are taken together with the atoms to which they are attached to form a C₃ cycloalkyl independently optionally substituted by R⁹, or

two R² are taken together with the atom to which they are attached to form a C₃ cycloalkyl, oxetanyl or tetrahydrofuranyl, each independently optionally substituted by R⁹, or

two R³ are taken together with the atom to which they are attached to form a C₃ cycloalkyl, oxetanyl or tetrahydrofuranyl, each independently optionally substituted by R⁹.

In certain embodiments, one R¹ and one R⁴ are taken together with the atoms to which they are attached to form a methylene or ethylene, independently optionally substituted by R⁹.

In certain embodiments, one R¹ and one R³ are taken together with the atoms to which they are attached to form a methylene or ethylene, independently optionally substituted by R⁹.

In certain embodiments, one R¹ and one R² are taken together with the atoms to which they are attached to form a fused C₃₋₆ cycloalkyl or 3-6 membered heterocyclyl, independently optionally substituted by R⁹. In certain embodiments, k and 1 are 2; one R¹ and one R² are taken together with the atoms to which they are attached to form a fused C₃₋₆ cycloalkyl or 3-6 membered heterocyclyl, independently optionally substituted by R⁹; and the other R¹ and R² are independently selected from hydrogen, halogen or C₁₋₃ alkyl optionally substituted by oxo or halogen.

In certain embodiments, one R² and one R³ are taken together with the atoms to which they are attached to form a fused C₃₋₆ cycloalkyl or 3-6 membered heterocyclyl, independently optionally substituted by R⁹. In certain embodiments, l and m are 2; one R² and one R³ are taken together with the atoms to which they are attached to form a fused C₃₋₆ cycloalkyl or 3-6 membered heterocyclyl, independently optionally substituted by R⁹, such as C₁-C₁₂ alkyl; and the other R² and R³ are independently selected from hydrogen, halogen or C₁₋₃ alkyl optionally substituted by oxo or halogen.

In certain embodiments, one R³ and one R⁴ are taken together with the atoms to which they are attached to form a fused C₃₋₆ cycloalkyl or 3-6 membered heterocyclyl, independently optionally substituted by R⁹. In certain embodiments, m and n are 2; one R³ and one R⁴ are taken together with the atoms to which they are attached to form a fused C₃₋₆ cycloalkyl or 3-6 membered heterocyclyl, independently optionally substituted by R⁹; and the other R³ and R⁴ are independently selected from hydrogen, halogen or C₁₋₃ alkyl optionally substituted by oxo or halogen.

In certain embodiments, R² is independently —OR⁷. In certain embodiments, R² is independently —OH or —OCH₃.

In certain embodiments, R² is independently 3-10-membered heterocyclyl independently optionally substituted by R⁹. In certain embodiments, R² is independently pyrazole or pyrimidinyl.

In certain embodiments, R² is independently C₁-C₁₂ alkyl independently optionally substituted by R⁹. In certain embodiments, R² is independently methyl, ethyl or methylhydroxy. In certain embodiments, 1 is 2; and R² is independently C₁-C₁₂ alkyl independently optionally substituted by R⁹.

In certain embodiments, R² is independently halogen. In certain embodiments, R² is independently fluoro.

In certain embodiments, two R² are taken together with the atom to which they are attached to form a C₃-C₆ cycloalkyl or 3-10-membered heterocyclyl, wherein said cycloalkyl and heterocyclyl are independently optionally substituted by R⁹,

In certain embodiments, two R² are taken together with the atom to which they are attached to form a C₃ cycloalkyl, oxetanyl or tetrahydrofuranyl.

In certain embodiments, R³ is independently 3-10-membered heterocyclyl independently optionally substituted by R⁹. In certain embodiments, R³ is independently pyrazole or pyrimidinyl.

In certain embodiments, R³ is independently C₁-C₁₂ alkyl independently optionally substituted by R⁹. In certain embodiments, R³ is independently methyl, ethyl or methylhydroxy.

In certain embodiments, R³ is independently halogen. In certain embodiments, R³ is independently fluoro.

In certain embodiments, two R³ are taken together with the atom to which they are attached to form a C₃-C₆ cycloalkyl or 3-10-membered heterocyclyl, wherein said cycloalkyl and heterocyclyl are independently optionally substituted by R⁹.

In certain embodiments, two R³ are taken together with the atom to which they are attached to form a C₃ cycloalkyl, oxetanyl or tetrahydrofuranyl.

In certain embodiments R⁵ is hydrogen. In certain embodiments R⁵ as hydrogen is excluded from any grouping of substituents.

In certain embodiments, R⁵ is C₁-C₆ alkylene or 3-10 membered heterocyclyl, optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —OCF₃, 3-10-membered heterocyclyl or 6-10 membered aryl (e.g., phenyl), wherein said alkyl, alkenyl, alkynyl, heterocyclyl and aryl are independently optionally substituted substituted by R²⁰.

In certain embodiments, R⁵ is C₁-C₆ alkylene optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —OCF₃, 3-10-membered heterocyclyl or 6-10 membered aryl (e.g., phenyl), wherein said alkyl, alkenyl, alkynyl, heterocyclyl and aryl are independently optionally substituted by R²⁰.

In certain embodiments, R⁵ is —CH₂—, —CH₂CH₂—, —CH₂CH₂CH₂—, —CH₂CH₂CH₂CH₂—, —CH(CH₃)—, or —CH(CH₂CH₃)—, wherein R⁵ is independently optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —OCF₃, 3-10-membered heterocyclyl or 6-10 membered aryl, wherein said alkyl, alkyenyl, alkynyl, heterocyclyl and aryl are independently optionally substituted by R²⁰.

In certain embodiments, R⁵ is —CH₂—, —CH₂CH₂—, or —CH₂CH₂CH₂—, wherein R⁵ is independently optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —OCF₃, 3-10-membered heterocyclyl or 6-10 membered aryl, wherein said alkyl, alkyenyl, alkynyl, heterocyclyl and aryl are independently optionally substituted by R²⁰.

In certain embodiments, R⁵ is C₁-C₆ alkylene optionally substituted by OH; halogen; —S(O)₂C₁₋₃ alkyl; pyrazolyl optionally substituted by C₁₋₃ alkyl; phenyl; azetidinyl optionally substituted by halogen, —S(O)₂C₁₋₃ alkyl, —C(O)C₁₋₃ alkyl or C₁₋₃ alkyl; piperidinyl optionally substituted by —C(O)C₁₋₃ alkyl, S(O)₂C₁₋₃ alkyl or C₁₋₃ alkyl optionally substituted by halogen or oxo; —NH₂; —NH(CH₃); —N(CH₃)₂; —NS(O)₂CH₃(CH₃); —N(oxetanyl)(CH₃); morpholinyl or tetrahydro-2H-thiopyranyl optionally substituted by oxo (e.g., ═O or (═O)₂).

In certain embodiments, R⁶ is 5-10-membered heterocyclyl or phenyl, wherein R⁶ is independently optionally substituted by R⁹. In certain embodiments, R⁶ is 4-6-membered heterocyclyl, wherein R⁶ is independently optionally substituted by R⁹, such as halogen or oxo In certain embodiments, R⁶ is a 6-membered heterocyclyl optionally substituted by oxo. In certain embodiments, R⁶ is tetrahydro-2H-thiopyranyl optionally substituted by oxo.

In certain embodiments, R⁶ is thianyl optionally substituted by R⁹, such as oxo or C₁-C₆ alkyl or C₁-C₆ haloalkyl. In certain embodiments, R⁶ is thietanyl 1,1-dioxide, 1,1-dioxothianyl, 1-oxothianyl, pyridinyl or phenyl independently optionally substituted by R⁹, such as —(C₀-C₆ alkylene)NR¹⁰R¹¹, wherein R¹⁰ and R¹¹ are, for example, each hydrogen. In certain embodiments, R⁶ is phenyl independently optionally substituted by —CN, C₁-C₆ alkyl optionally substituted by halogen or halogen. In certain embodiments, R⁶ is phenyl independently optionally substituted by —CN, Cl, F, methyl or trifluoromethyl.

In certain embodiments, R⁶ is not a 3-10-membered heterocyclyl. In certain embodiments, R⁶ is not a 6-10-membered aryl. In certain embodiments, R⁶ is substituted by more than one R⁹.

In some embodiments, R⁵ is C₁-C₆ alkylene optionally substituted by an optionally substituted 6-10 membered aryl (e.g., phenyl) and R⁶ is optionally substituted 1,1-dioxothianyl or 1-oxothianyl.

In certain embodiments, R⁵-R⁶ together do not constitute C₁-C₆ alkyl. In certain embodiments, R⁵-R⁶ together do not constitute —CH₃.

In certain embodiments, each R⁷ and R⁸ are independently hydrogen or methyl.

In certain embodiments, each R⁹ is independently hydrogen, C₁-C₁₂ alkyl, C₂-C₁₂ alkynyl, halogen, —CN, —(C₀-C₆ alkylene)OR¹⁰, —(C₀-C₆ alkylene)NR¹⁰R¹¹, —CF₃, —(C₀-C₆ alkylene)C(O)OR¹⁰, —(C₀-C₆ alkylene)C(O)NR¹⁰R¹¹, —(C₀-C₆ alkylene)(5-6-membered heterocyclyl), —(C₀-C₆ alkylene)C(O)(5-6-membered heterocyclyl) or phenyl, wherein each R⁹ is independently optionally substituted by halogen, oxo, —CF₃, —CN, —OR¹², —SR¹², —NR¹²R¹³, —C(O)R¹², —S(O)₁₋₂R¹², C₁-C₆ alkyl optionally substituted by oxo or halogen, C₂-C₆ alkenyl optionally substituted by oxo or halogen, or C₂-C₆ alkynyl optionally substituted by oxo or halogen.

In certain embodiments, each R¹⁶ and R¹⁷ are independently hydrogen, C₁₋₃ alkyl, 3-6 membered heterocyclyl, S(O)₂C₁₋₃alkyl or cyclopropyl, wherein said alkyl, heterocyclyl and cyclopropyl are independently optionally substituted by halogen, oxo, —CF₃, —OCF₃, —OR¹⁸, —SR¹⁸, —NR¹⁸R¹⁹, —CN, 3-6-membered heterocyclyl, phenyl, C₃-C₆ cycloalkyl or C₁-C₆ alkyl optionally substituted by halogen or oxo.

In certain embodiments, each R¹⁶ and R¹⁷ are independently hydrogen or methyl.

In certain embodiments, each R²⁰ is independently hydrogen, halogen, C₁₋₃ alkyl, —C(O)C₁₋₃alkyl or —S(O)₂C₁₋₃alkyl.

In certain embodiments, ring A is a 6-membered cycloalkyl; (a) p is 2 and each R^{a} is methyl where each methyl group is bound to the same ring A atom or (b) p is 3, where two R^{a} are taken together with the atoms to which they are attached to form a C₃-C₆ cycloalkyl and one R^{a} is methyl; R⁵ is C₁-C₆ alkylene optionally substituted by (i) SR¹⁶ or NR¹⁶R¹⁷, wherein each R¹⁶ is —S(O)₁₋₂C₁-C₆ alkyl or H and R¹⁷ is H; (ii) 3-10-membered heterocyclyl; or (iii) 6-10 membered aryl, wherein said heterocyclyl is optionally substituted by oxo; R⁶ is hydrogen or 3-10-membered heterocyclyl optionally substituted by R⁹, wherein R⁹ is OXO.

Another aspect includes compounds of formula (III):

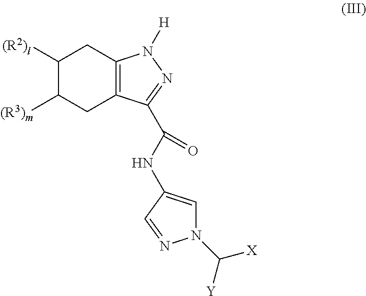

or stereoisomers or a pharmaceutically acceptable salt thereof, wherein:

l is 1 or 2 and m is 0 or 1;

each R² is C₁-C₆ alkyl (e.g., methyl) or R² and R³ together with the atoms to which they are attached form an optionally substituted (e.g., halogen) C₃-C₆ cycloalkyl (e.g., cyclopropyl);

X is 3-10-membered heterocyclyl (e.g., pyrimidinyl) or 6-10 membered aryl (e.g., phenyl), or C₂-C₆ alkylene, each optionally substituted by OH; halogen; —S(O)₂C₁₋₃ alkyl; C₁₋₃ alkyl optionally substituted by halogen or oxo; —C(O)C₁₋₃ alkyl; —NH₂; —NH(CH₃); —N(CH₃)₂; —NS(O)₂CH₃(CH₃); —N(oxetanyl)(CH₃); morpholinyl or tetrahydro-2H-thiopyranyl optionally substituted by oxo; and

Y is H or a 3-10-membered heterocyclyl optionally substituted by oxo.

In some embodiments, a compound of formula (AA), (A), (I), (II) or (III) is further defined as a compound of formula (IIIa) or formula (IIIb):

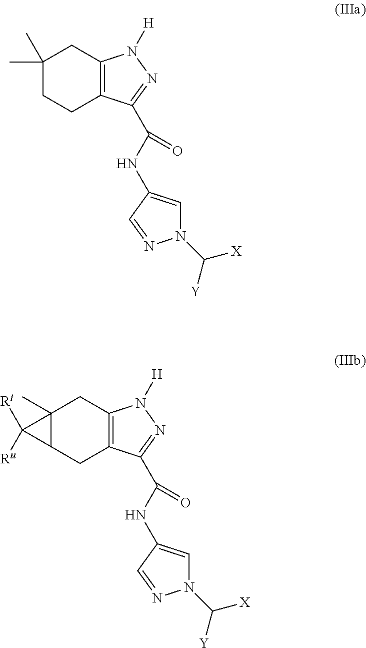

or stereoisomers or a pharmaceutically acceptable salt there, wherein:

R^{u} is hydrogen or halogen (e.g., fluoro);

R^{t} is hydrogen or halogen (e.g., fluoro);

X is an optionally substituted 6-10 membered aryl (e.g., phenyl); and

Y is an optionally substituted 3-10-membered heterocyclyl, such as 1,1-dioxothianyl or 1-oxothianyl.

Another aspect includes a compound selected from Examples 1-154b, stereoisomers or a pharmaceutically acceptable salt thereof.

Another aspect includes a prodrug of compounds the present invention. A “prodrug” is a compound that may be converted under physiological conditions or by solvolysis to the specified compound or to a salt of such compound. Prodrugs include compounds wherein an amino acid residue, or a polypeptide chain of two or more (e.g., two, three or four) amino acid residues, is covalently joined through an amide or ester bond to a free amino, hydroxy or carboxylic acid group of a compound of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, cirtulline, homocysteine, homoserine, methyl-alanine, para-benzoylphenylalanine, phenylglycine, propargylglycine, sarcosine, methionine sulfone and tert-butylglycine.

Additional types of prodrugs are also encompassed. For instance, a free carboxyl group of a compound of the present invention can be derivatized as an amide or alkyl ester. As another example, compounds of this invention comprising free hydroxy groups may be derivatized as prodrugs by converting the hydroxy group into a group such as, but not limited to, a phosphate ester, hemisuccinate, dimethylaminoacetate, or phosphoryloxymethyloxycarbonyl group, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group may be an alkyl ester optionally substituted by groups including, but not limited to, ether, amine and carboxylic acid functionalities, or where the acyl group is an amino acid ester as described above, are also encompassed. Prodrugs of this type are described in J. Med. Chem., 1996, 39, 10. More specific examples include replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxy-carbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

Another aspect includes isotopically-labeled compounds of the present invention, which are structurally identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically-labeled compounds of the present invention (e.g., those labeled with ³H and ¹⁴C) are useful in compound or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Another aspect includes salts of compounds of the present invention. Examples of salts include those salts prepared by reaction of a compound of the present invention with a mineral or organic acid or an inorganic base, such salts including, but not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyn-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methanesulfonates, propanesulfonates, naphthalene-1-sulfonates, naphthalene-2-sulfonates, and mandelates. Since a single compound of the present invention may include more than one acidic or basic moiety, the compounds of the present invention may include mono, di or tri-salts in a single compound. In one example, the salt is a pharmaceutically acceptable acid addition salt. In another example, the salt is a pharmaceutically acceptable base addition salt.

The compounds of the present invention also include other salts of such compounds which are not necessarily pharmaceutically acceptable salts, and which may be useful as intermediates for preparing and/or purifying compounds of the present invention or for separating enantiomers of compounds of the present invention.

Another aspect includes the in vivo metabolic products of compounds of the present invention described herein. A “metabolite” is a pharmacologically active product produced through metabolism in the body of a specified compound or salt thereof. Such products may result, for example, from the oxidation, reduction, hydrolysis, amidation, deamidation, esterification, deesterification, enzymatic cleavage, glucuronidation, and the like, of the administered compound. Accordingly, another aspect includes metabolites of compounds of the present invention, including compounds produced by a process comprising contacting a compound of this invention with a mammal for a period of time sufficient to yield a metabolic product thereof.

Metabolites are identified, for example, by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to a human, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolites, so long as they are not otherwise found in vivo, are useful in diagnostic assays for therapeutic dosing of the compounds of the invention.

Synthesis of ITK Inhibitor Compounds

Compounds of this invention may be synthesized by synthetic routes that include processes analogous to those well known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), or Beilsteins Handbuch der organischen Chemie, 4, Aufl. ed. Springer-Verlag, Berlin, including supplements).

Compounds of the present invention may be prepared singly or as compound libraries comprising 2 or more compounds, for example 5 to 1,000 compounds, or 10 to 100 compounds. Libraries of compounds of the present invention may be prepared by a combinatorial ‘split and mix’ approach or by multiple parallel syntheses using either solution phase or solid phase chemistry, by procedures known to those skilled in the art. Thus according to a further aspect of the invention there is provided a compound library comprising at least 2 compounds of the present invention.

For illustrative purposes, the below schemes show a general method for preparing the compounds of the present invention as well as intermediates. For a more detailed description of the individual reaction steps, see the Examples section below. Those skilled in the art will appreciate that other synthetic routes may be used to synthesize the compounds described herein. Although specific starting materials and reagents are depicted in the Schemes and discussed below, other starting materials and reagents can be easily substituted to provide a variety of derivatives or reaction conditions.

Compounds of the present invention can be prepared, using an amide bond forming reaction as the key step, for example as shown below (wherein q is 1 or 2, and the other variables are defined herein for formulas (AA), (A), (I), (II), (IIa), (IIb), (III), (IIIa) and (IIIb):

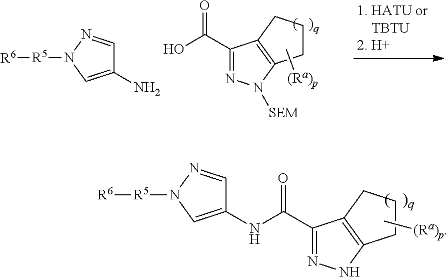

The aminopyrazole fragments can be prepared by one of the two following methods:

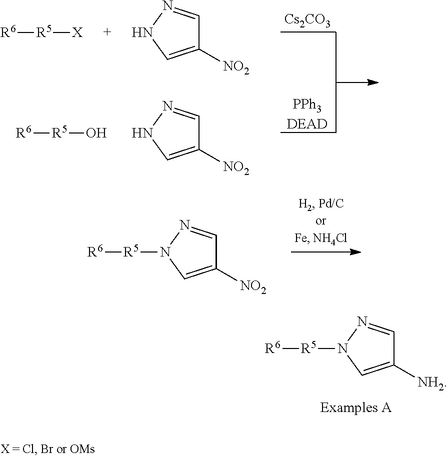

The pyrazole carboxylate fragments were also prepared by one of two methods, giving differential regioselectivity:

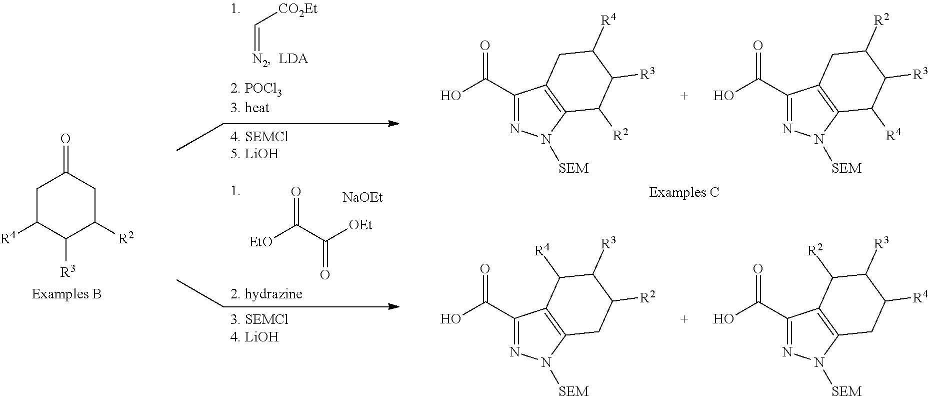

In some embodiments, the invention provides a process for manufacturing a compound of formula (AA), comprising contacting a compound of formula (i), or salt thereof, with a compound of formula (ii), or salt thereof:

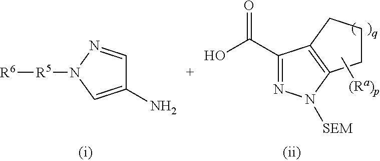

to form a compound of formula (AA) or salt thereof.

### Pharmaceutical Compositions and Administration

Another embodiment provides pharmaceutical compositions or medicaments containing the compounds of the present invention and a therapeutically inert carrier, diluent or excipient, as well as methods of using the compounds of the invention to prepare such compositions and medicaments. In one example, compounds of the present invention may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound. In some embodiments, pH ranges anywhere from about 3 to about 8. In one example, a compound of the present invention is formulated in an acetate buffer, at pH 5. In another embodiment, the compounds of the present invention are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit ITK kinase activity in a cell. For example, such amount may be below the amount that is toxic to normal cells, or the mammal as a whole.

In one example, the pharmaceutically effective amount of the compound of the invention administered parenterally per dose will be in the range of about 0.01-100 mg/kg, alternatively about 0.1 to 20 mg/kg of patient body weight per day, with the typical initial range of compound used being 0.3 to 15 mg/kg/day. In another embodiment, oral unit dosage forms, such as tablets and capsules, contain from about 25-100 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 mg, 50 mg, 100 mg, 250 mg, or 500 mg of the compound of the invention compounded with about 30-90 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5-400 mg, of the invention in a suitable buffer solution, e.g., a phosphate buffer, adding a tonicifier, e.g., a salt such sodium chloride, if desired. The solution may be filtered, e.g., using a 0.2 micron filter, to remove impurities and contaminants.

One aspect, therefore, includes a pharmaceutical composition comprising a compound of the present invention, or a stereoisomer or pharmaceutically acceptable salt thereof. A further embodiment includes a pharmaceutical composition comprising a compound of the present invention, or a stereoisomer or pharmaceutically acceptable salt thereof, together with a pharmaceutically acceptable carrier or excipient.

Another embodiment includes a pharmaceutical composition comprising a compound of the present invention for use in the treatment of a disease responsive to the inhibition of ITK kinase. Another embodiment includes a pharmaceutical composition comprising a compound of the present invention for use in the treatment of a immunological or inflammatory disease. Another embodiment includes a pharmaceutical composition comprising a compound of the present invention for use in the treatment of asthma or atopic dermatitis.

Indications and Methods of Treatment

ITK is activated downstream of antigen engagement of the T cell receptor (TCR) and mediates TCR signals through the phosphorylation and activation of PLCg. Mice in which ITK is deleted showed defective differentiation of T cells towards the Th2 subset, but not the Th1 subset. Additional studies indicate that Th2 cytokine production, but not early Th2 lineage commitment, is defective in ITK-deficient mouse T cells. Th2 cells promote allergic inflammation, and ITK knock-out mice have reduced lung inflammation, mucus production, and airway hyperreactivity in models of allergic asthma.

The compounds of the invention inhibit the activity of ITK kinase. Accordingly, the compounds of the invention are useful for the treatment of inflammation and immunological diseases. Inflammatory diseases which can be treated according to the methods of this invention include, but are not limited to, asthma, allergic rhinitis, atopic dermatitis, rheumatoid arthritis, psoriasis, contact dermatitis, and delayed hypersensitivity reactions.

An embodiment includes a method of treating or preventing a disease responsive to the inhibition of ITK kinase in a mammal in need of such treatment, wherein the method comprises administering to said mammal a therapeutically effective amount of a compound of the present invention, a stereoisomer or pharmaceutically acceptable salt thereof.

An embodiment includes use of a compound of the present invention, a stereoisomer or pharmaceutically acceptable salt thereof in therapy.

Another embodiment includes a compound of the present invention, a stereoisomer or pharmaceutically acceptable salt thereof for use in therapy.

Another embodiment includes use of a compound of the present invention, a stereoisomer or pharmaceutically acceptable salt thereof in treating or preventing a disease responsive to the inhibition of ITK kinase.

Another embodiment includes use of a compound of the present invention, a stereoisomer or pharmaceutically acceptable salt thereof in treating or preventing an inflammatory disease.

Another embodiment includes use of a compound of the present invention, a stereoisomer or pharmaceutically acceptable salt thereof in treating asthma, allergic rhinitis, atopic dermatitis, rheumatoid arthritis, psoriasis, contact dermatitis, and delayed hypersensitivity reactions. A further embodiment includes a method of using of a compound described herein in a dose ranging from 25-500 mg for such treatments.

Another embodiment includes use of a compound of the present invention, a stereoisomer or pharmaceutically acceptable salt thereof in the manufacture of a medicament for the treatment of an inflammatory disease. A further embodiment includes using of a compound described herein in a dose ranging from 25-500 mg in such uses.

Another embodiment includes use of a compound of the present invention, a stereoisomer or pharmaceutically acceptable salt thereof in the manufacture of a medicament for the treatment of asthma, allergic rhinitis, atopic dermatitis, rheumatoid arthritis, psoriasis, contact dermatitis, and delayed hypersensitivity reactions. A further embodiment includes using of a compound described herein in a dose ranging from 25-500 mg for such treatments.

Compounds of the invention are also useful for reducing inflammation in cells that overexpress ITK. Alternatively, compounds of the invention are useful for reducing inflammation in cells that have aberrant or overactive antigen engagement of the T cell receptor. Alternatively, compounds of the invention are useful for reducing inflammation in cells that have over-activation or phosphorylation of PLCg. Additionally, the compounds can be used for the treatment of inflammation or immunological disorders in cells that overexpress Th2 cytokine. Another embodiment includes a method of treating or preventing cancer in a mammal in need of such treatment, wherein the method comprises administering to said mammal a therapeutically effective amount of a compound of the present invention, a stereoisomer or pharmaceutically acceptable salt thereof.

### Combination Therapy

The compounds of the present invention may be employed alone or in combination, such as with other chemotherapeutic agents for treatment. The compounds of the present invention can be used in combination with one or more additional drugs, for example an anti-hyperproliferative, anti-cancer, cytostatic, cytotoxic, anti-inflammatory or chemotherapeutic agents. The second compound of the pharmaceutical combination formulation or dosing regimen typically has complementary activities to the compound of this invention such that they do not adversely affect each other. Such agents are suitably present in combination in amounts that are effective for the purpose intended. The compounds may be administered together in a unitary pharmaceutical composition or separately and, when administered separately this may occur simultaneously or sequentially. Such sequential administration may be close or remote in time. In one embodiment, compounds of the present invention are coadministered with a cytostatic compound selected from the group consisting of cisplatin, doxorubicin, taxol, taxotere and mitomycin C. In another embodiment, the cytostatic compound is doxorubicin. In another embodiment, compounds of the present invention are coadministered with an anti-inflammatory agent selected from a NSAID and corticosteroid. In one embodiment, compounds of the present invention are coadministered with an anti-asthmtic agents, including but not limited to beta2-adrenergic agonists, inhaled and oral corticosteroids, leukotriene receptor antagonist, and omalizumab. In another embodiment, compounds of the present invention are coadministered with an anti-asthmatic agent selected from a NSAID, combinations of fluticasone and salmeterol, combinations of budesonide and formoterol, omalizumab, lebrikizumab and corticosteroid selected from fluticasone, budesonide, mometasone, flunisolide and beclomethasone. In another embodiment, compounds of the present invention are coadministered with an anti-rheumatoid agent, in one example, RITUXAN®. In another embodiment, compounds of the present invention are coadministered with a chemotherapeutic agent selected from etanercept (Enbrel), infliximab (Remicade), adalimumab (Humira), certolizumab pegol (Cimzia), golimumab (Simponi), Interleukin 1 (IL-1) blockers such as anakinra (Kineret), monoclonal antibodies against B cells such as rituximab (RITUXAN®), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa).

The compounds of the present invention can be also used in combination with radiation therapy. The phrase “radiation therapy” refers to the use of electromagnetic or particulate radiation in the treatment of neoplasia. Radiation therapy delivers doses of radiation sufficiently high to a target area to cause death of reproducing cells, in both tumor and normal tissues. The radiation dosage regimen is generally defined in terms of radiation absorbed dose (rad), time and fractionation, and must be carefully defined by the oncologist. The amount of radiation a patient receives will depend on various considerations but two of the most important considerations are the location of the tumor in relation to other critical structures or organs of the body, and the extent to which the tumor has spread. Examples of radiotherapeutic agents are provided in Hellman, Principles of Radiation Therapy, Cancer, in Principles I and Practice of Oncology, 24875 (Devita et al., 4th ed., vol 1, 1993). Alternative forms of radiation therapy include three-dimensional conformal external beam radiation, intensity modulated radiation therapy (IMRT), stereotactic radiosurgery and brachytherapy (interstitial radiation therapy), the latter placing the source of radiation directly into the tumor as implanted “seeds”. These alternative treatment modalities deliver greater doses of radiation to the tumor, which accounts for their increased effectiveness when compared to standard external beam radiation therapy.

### Articles of Manufacture

Another embodiment includes a kit for treating a disease or disorder responsive to the inhibition of ITK kinase. The kit includes:

(a) a first pharmaceutical composition comprising a compound of the present invention; and

(b) instructions for use.

In another embodiment, the kit further includes:

(c) a second pharmaceutical composition, which includes a chemotherapeutic agent.

In one embodiment, the instructions describe the simultaneous, sequential or separate administration of said first and second pharmaceutical compositions to a patient in need thereof.

In one embodiment, the first and second compositions are contained in separate containers.

In one embodiment, the first and second compositions are contained in the same container.

Containers for use include, for example, bottles, vials, syringes, blister pack, etc. The containers may be formed from a variety of materials such as glass or plastic. The container includes a compound of the present invention or formulation thereof which is effective for treating the condition and may have a sterile access port (for example the container may be an intravenous solution bag or a vial having a stopper pierceable by a hypodermic injection needle). The container includes a composition comprising at least one compound of the present invention. The label or package insert indicates that the composition is used for treating the condition of choice, such as cancer. In one embodiment, the label or package inserts indicates that the composition comprising the compound of the present invention can be used to treat a disorder. In addition, the label or package insert may indicate that the patient to be treated is one having a disorder characterized by overactive or irregular kinase activity. The label or package insert may also indicate that the composition can be used to treat other disorders.

The article of manufacture may comprise (a) a first container with a compound of the present invention contained therein; and (b) a second container with a second pharmaceutical formulation contained therein, wherein the second pharmaceutical formulation comprises a chemotherapeutic agent. The article of manufacture in this embodiment of the invention may further comprise a package insert indicating that the first and second compounds can be used to treat patients at risk of stroke, thrombus or thrombosis disorder. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare other compounds of the present invention, and alternative methods for preparing the compounds of the present invention are within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

### EXAMPLES

The invention will be more fully understood by reference to the following examples. They should not, however, be construed as limiting the scope of the invention.

### Intermediate Examples

### Synthesis of Aminopyrazoles

### Examples A

### Example A1

### 3-((4-amino-1H-pyrazol-1-yl)methyl)benzonitrile

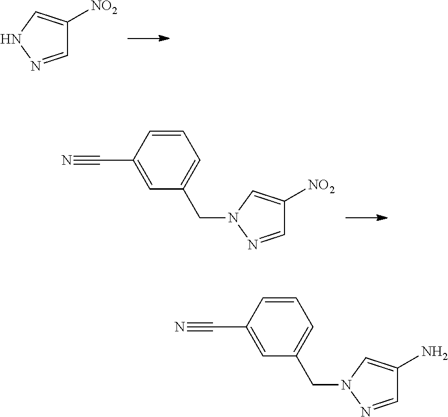 [SMILES: O=[N+]([O-])c1cn[nH]c1 ; N#Cc1cccc(Cn2cc([N+](=O)[O-])cn2)c1 ; N#Cc1cccc(Cn2cc(N)cn2)c1]

To a solution of 4-nitro-1H-pyrazole (4.00 g, 35.4 mmol) in N,N-dimethylformamide (200 mL) was added K₂CO₃ (5.867 g, 42.45 mmol), then m-cyanobenzyl bromide (6.935 g, 35.37 mmol). The mixture was stirred overnight at rt then the mixture was diluted with 300 mL EtOAc and washed with 2×200 mL 1:1 H₂O:brine. The organic extracts were dried (Na₂SO₄) and concentrated in vacuo. Purification by CombiFlash (120 g column; dry load; 0:100 EtOAc/heptane over 32 minutes) provided 7.60 g (95%) of the title compound as a white solid. To a solution of 3-((4-nitro-1H-pyrazol-1-yl)methyl)benzonitrile (1.38 g, 6.06 mmol) in ethanol (40 mL) was added ammonium chloride (1.62 g, 30.3 mmol) as a saturated solution in water then iron (1.69 g, 30.3 mmol). The mixture was heated to 80° C. for 60 minutes, then cooled to rt. The mixture was diluted with 150 mL EtOAc and washed with 100 mL sat. NaHCO₃(aq) and 100 mL brine. The organic extracts were dried (Na₂SO₄) and concentrated in vacuo. The unpurified residue (1.20 g; quant.) was used directly without further purification.

### Example A2

### 1-benzyl-1H-pyrazol-4-amine

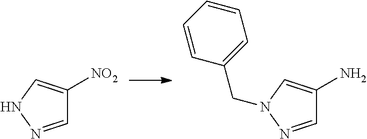 [SMILES: O=[N+]([O-])c1cn[nH]c1 ; Nc1cnn(Cc2ccccc2)c1]

Prepared in an analogous manner to 3-((4-amino-1H-pyrazol-1-yl)methyl)benzonitrile (Example A1), replacing m-cyanobenzyl bromide with benzyl bromide.

### Example A3

### 1-(3-(dimethylamino)-1-phenylpropyl)-1H-pyrazol-4-amine

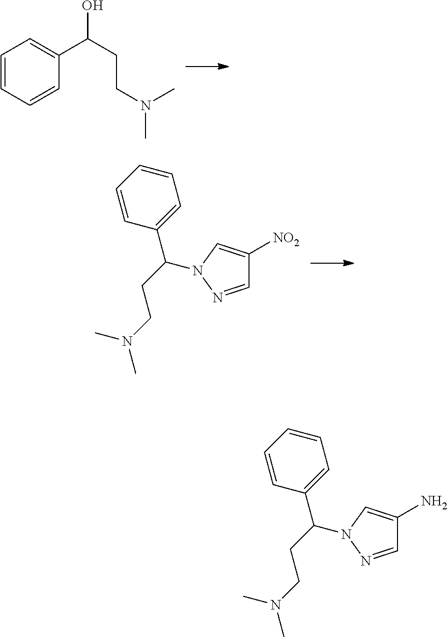

3-(Dimethylamino)-1-phenyl-propan-1-ol (4.59 g, 25.6 mmol; see Synthesis, 2003, 1626) in dry THF (50 mL) at 0° C. was added 4-nitro-1H-pyrazole (2.95 g, 25.6 mmol) followed by triphenylphosphine (13.7 g, 51.2 mmol) and then diethyl azodicarboxylate (23.3 mL, 51.2 mmol, 40 mass %) dropwise. The sample was allowed to warm slowly to room temperature and stirred overnight. The sample was diluted with H₂O, extracted 3 times with EtOAc, washed once with sat NaCl, dried over MgSO₄, filtered and concentrated in vacuo. The sample was chromatographed through silica gel (330 g, 0-100% EtOAc in heptane then 10% MeOH in dichloromethane to provide N,N-dimethyl-3-(4-nitro-1H-pyrazol-1-yl)-3-phenylpropan-1-amine (quant; contains some PPh₃) which was used directly without further purification. This material was diluted with 70 mL EtOH, then 10% palladium on carbon (1.1 g) was added and the mixture was stirred under an atmosphere of hydrogen overnight. The sample was purged with nitrogen, filtered through Celite, and concentrated in vacuo to provide the title compound which was used directly without further purification.

### Example A4

### (S)-1-(1-phenylpropyl)-1H-pyrazol-4-amine

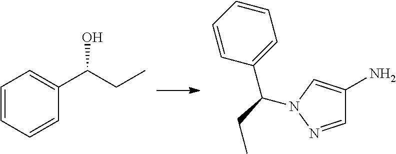

Prepared in an analogous manner to 1-(3-(dimethylamino)-1-phenylpropyl)-1H-pyrazol-4-amine (Example A2), replacing 3-(dimethylamino)-1-phenyl-propan-1-ol with (R)-1-phenylpropan-1-ol (commercial).

### Example A5

### 1-(2-(dimethylamino)-1-phenylethyl)-1H-pyrazol-4-amine

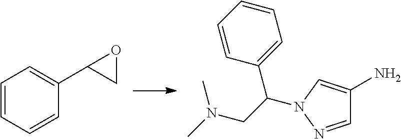 [SMILES: c1ccc(C2CO2)cc1 ; CN(C)CC(c1ccccc1)n1cc(N)cn1]

Sodium hydroxide (1.5 equiv., 24.7 mmol, 999 mg) was suspended in water (6 mL) and stirred until dissolved. Ethanol (10 mL) and dimethylamine hydrochloride (1.5 equiv., 25.0 mmol, 2.06 g) were added, followed by 2-phenyloxirane (2 g, 16.646 mmol), and the mixture was stirred for 3 hours at rt. The mixture was diluted with 100 mL EtOAc and washed with 50 mL water. The organic extracts were dried (Na₂SO₄) and concentrated in vacuo. Purification by CombiFlash (80 g; dry load; 100:0 to 90:10:1 CH₂Cl₂:NH₄OH over 40 minutes) provided a 1.5 grams of a ˜2.5:1 mixture of 2-(dimethylamino)-1-phenylethanol and 2-(dimethylamino)-2-phenylethanol.

This mixture was diluted with tetrahydrofuran (30 mL), then to this solution was added 4-nitro-1H-pyrazole (1.5 equiv., 14.3 mmol, 1600 mg), triphenylphosphine (1.5 equiv., 14.3 mmol, 3.82 g) then diisopropyl azodicarboxylate (1.5 equiv., 14.3 mmol, 3.04 g, 2.96 mL). The mixture was stirred overnight at rt, then concentrated in vacuo. Purification by CombiFlash (80 g; dry load; 100:0 to 95:5:0.5 CH₂Cl₂:MeOH:NH₄OH over 40 minutes) provided a mixture of N,N-dimethyl-2-(4-nitro-1H-pyrazol-1-yl)-2-phenylethanamine and triphenylphopshine oxide. Nitropyrazole reduction was accomplished using palladium on carbon under an atmosphere of hydrogen, as outlined in Example A3.

### Example A6

### Tert-butyl 4-((4-amino-1H-pyrazol-1-yl)(phenyl)methyl)-4-fluoropiperidine-1-carboxylate

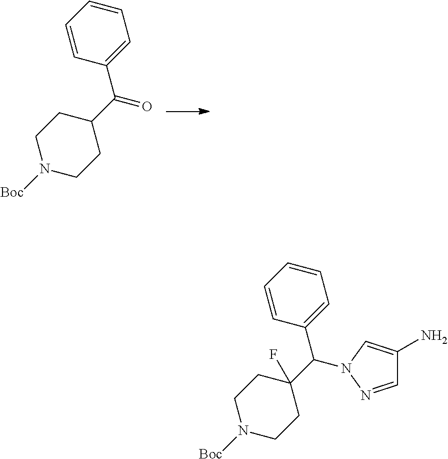 [SMILES: CC(C)(C)OC(=O)N1CCC(C(=O)c2ccccc2)CC1 ; CC(C)(C)OC(=O)N1CCC(F)(C(c2ccccc2)n2cc(N)cn2)CC1]

A solution of tert-butyl 4-benzoylpiperidine-1-carboxylate (486 mg, 1.679 mmol, 486 mg; see J. Med. Chem. 2000, 43, 3878) in tetrahydrofuran (10 mL) was cooled to −78° C., then lithium hexamethyldisilazide (1 mol/L) in THF (1.3 equiv., 2.18 mmol, 2.18 mL) was added dropwise. The mixture was warmed to 0° C., stirred for 30 minutes, then cooled back to −78° C. n-Fluoro-n-(phenylsulfonyl)benzenesulfonamide (1.3 equiv., 2.18 mmol, 725 mg) was added dropwise as a solution in 2 mL THF, then the mixture was stirred overnight while slowly warming to rt. The reaction was poured into 50 mL brine, and extracted with 50 mL EtOAc. The combined organic extracts were dried (Na₂SO₄) and concentrated in vacuo. Purification by CombiFlash (40 g; dry load; 100:0 to 60:40 heptane:EtOAc over 20 minutes) provided tert-butyl 4-benzoyl-4-fluoro-piperidine-1-carboxylate (403 mg, 1.31 mmol, 78% yield).

To a solution of tert-butyl 4-benzoyl-4-fluoro-piperidine-1-carboxylate (403 mg, 1.311 mmol) in tetrahydrofuran (5 mL) and methanol (5 mL) was added sodium borohydride (1.5 equiv., 1.97 mmol, 75.9 mg) and the mixture was stirred for 60 minutes at rt. The reaction was quenched by the addition of ˜5 mL sat. NH₄Cl (aq), then the mixture was diluted with 50 mL brine and extracted with 50 mL EtOAc. The organic extracts were dried (Na₂SO₄) and concentrated in vacuo to provide tert-butyl 4-fluoro-4-[hydroxy(phenyl)methyl]piperidine-1-carboxylate (405 mg, 1.31 mmol, 99% yield). This alcohol was converted to an aminopyrazole via a Mitsonobu reaction followed by palladium on carbon reduction as is outlined in Example A3.

### Example A7

### 1-((1-(methylsulfonyl)azetidin-3-yl)(phenyl)methyl)-1H-pyrazol-4-amine COOH

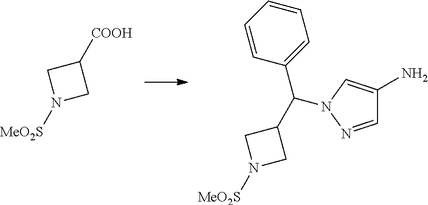 [SMILES: CS(=O)(=O)N1CC(C(=O)O)C1 ; CS(=O)(=O)N1CC(C(c2ccccc2)n2cc(N)cn2)C1]

To a solution of 1-methylsulfonylazetidine-3-carboxylic acid (200 mg, 1.1161 mmol, commercial) in N,N-dimethylformamide (5 mL) was added N,O-dimethylhydroxylamine hydrochloride (1.5 equiv., 1.67 mmol, 163 mg), HATU (1.5 equiv., 1.67 mmol, 636 mg) and diisopropylethylamine (3.0 equiv., 3.34 mmol, 0.59 mL). The mixture was stirred for 2 days, then diluted with 50 mL EtOAc and washed with 50 mL sat. NaHCO₃ (aq) and 2×50 mL 1:1 H₂O:brine. The organic extracts were dried (Na₂SO₄) and concentrated in vacuo to provide N-methoxy-N-methyl-1-methylsulfonyl-azetidine-3-carboxamide (100 mg, 0.45 mmol, 40% yield) of sufficient purity to be used directly.

This material was diluted with tetrahydrofuran (2 mL) and cooled to 0° C., then phenylmagnesium bromide (3.0 mol/L) in diethyl ether (2 equiv., 0.90 mmol, 0.30 mL) was added dropwise. The mixture was stirred for 2 hours, while slowly warming to rt. The reaction was quenched by the addition of ˜2 mL sat. NH₄Cl (aq), then the mixture was diluted with 50 mL EtOAc and washed with 50 mL brine. The organic extracts were dried (Na₂SO₄) and concentrated in vacuo. Purification by CombiFlash (12 g; dry load; 80:20 to 40:60 heptane:EtOAc over 20 minutes) provided (1-methylsulfonylazetidin-3-yl)-phenyl-methanone (39 mg, 0.16 mmol, 36% yield).

To a solution of (1-methylsulfonylazetidin-3-yl)-phenyl-methanone (39 mg, 0.1630 mmol, 39 mg) in methanol (1 mL) and tetrahydrofuran (1 mL) was added sodium borohydride (1.5 equiv., 0.24 mmol, 9.4 mg) and the mixture was stirred for 90 minutes at rt. The reaction was quenched by the addition of ˜2 mL sat. NH₄Cl (aq), then the mixture was diluted with 50 mL EtOAc and washed with 50 mL brine. The organic extracts were dried (Na₂SO₄) and concentrated in vacuo to provide (1-methylsulfonylazetidin-3-yl)-phenyl-methanol (39 mg, 0.1616 mmol, 99% yield). This alcohol was converted to an aminopyrazole via a Mitsonobu reaction followed by palladium on carbon reduction as is outlined in Example A3.

### Example A8

### tert-butyl 3-((4-amino-1H-pyrazol-1-yl)(phenyl)methyl)-3-fluoroazetidine-1-carboxylate

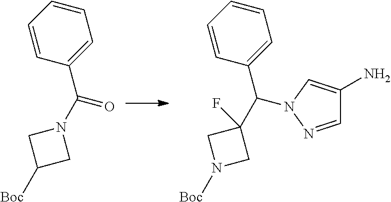 [SMILES: CC(C)(C)OC(=O)C1CN(C(=O)c2ccccc2)C1 ; CC(C)(C)OC(=O)N1CC(F)(C(c2ccccc2)n2cc(N)cn2)C1]

Prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoylpiperidine-1-carboxylate with tert-butyl 3-benzoylazetidine-1-carboxylate (see Synlett 1998, 379).

### Example A9

### tert-butyl (3-(4-amino-1H-pyrazol-1-yl)-3-phenylpropyl)(methyl)carbamate

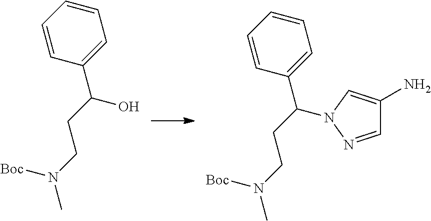 [SMILES: CN(CCC(O)c1ccccc1)C(=O)OC(C)(C)C ; CN(CCC(c1ccccc1)n1cc(N)cn1)C(=O)OC(C)(C)C]

Prepared in an analogous manner to 1-(3-(dimethylamino)-1-phenylpropyl)-1H-pyrazol-4-amine (Example A3), replacing 3-(dimethylamino)-1-phenyl-propan-1-ol with tert-butyl (3-hydroxy-3-phenylpropyl)(methyl)carbamate (see WO2008/98104 A1).

### Example A10

### 1-(1-(pyridin-3-yl)propyl)-1H-pyrazol-4-amine

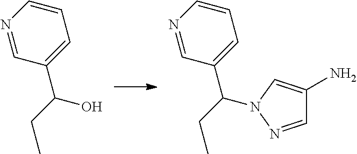

Prepared in an analogous manner 1-(3-(dimethylamino)-1-phenylpropyl)-1H-pyrazol-4-amine (Example A3), replacing 3-(dimethylamino)-1-phenyl-propan-1-ol with 1-(pyridin-3-yl)propan-1-ol (see J. Chem. Soc. 1963, 4269).

### Example A11

### methyl 2-(4-amino-1H-pyrazol-1-yl)-2-phenylacetate

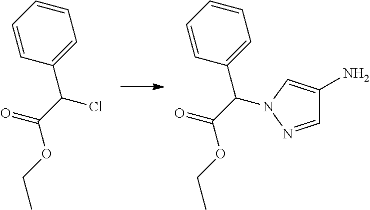 [SMILES: CCOC(=O)C(Cl)c1ccccc1 ; CCOC(=O)C(c1ccccc1)n1cc(N)cn1]

To a solution of ethyl 2-chloro-2-phenyl-acetate (501 mg, 2.52 mmol) and 4-nitro-1H-pyrazole (1.1 equiv., 2.77 mmol, 313 mg) in dimethylformamide (5 mL) was added cesium carbonate (1.1 equiv., 2.77 mmol, 904 mg) and the mixture was stirred overnight at rt. The mixture was diluted with 50 mL EtOAc, and washed with 2×50 mL 1:1 H₂O:brine. The organic extracts were dried (Na₂SO₄) and concentrated in vacuo. Purification by CombiFlash (40 g; dry load; 100:0 to 50:50 heptane:EtOAc over 20 minutes) provided ethyl 2-(4-nitropyrazol-1-yl)-2-phenyl-acetate (694 mg, 2.52 mmol, 99% yield). Nitropyrazole reduction is accomplished using palladium on carbon as outlined in Example A3.

### Example A12

### 1-(2-(azetidin-1-yl)-1-phenylethyl)-1H-pyrazol-4-amine

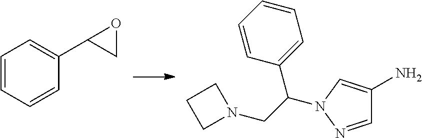 [SMILES: c1ccc(C2CO2)cc1 ; Nc1cnn(C(CN2CCC2)c2ccccc2)c1]

Prepared in an analogous manner to 1-(2-(dimethylamino)-1-phenylethyl)-1H-pyrazol-4-amine (Example A5), replacing dimethylamine hydrochloride with azetidine.

### Example A13

### N-(2-(4-amino-1H-pyrazol-1-yl)-2-phenylethyl)-N-methylmethanesulfonamide

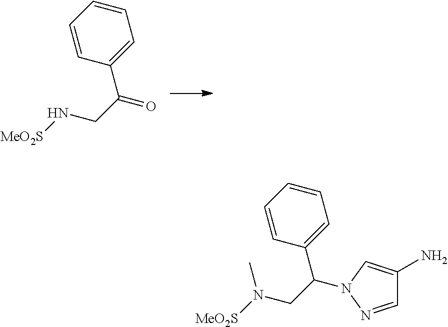 [SMILES: CS(=O)(=O)NCC(=O)c1ccccc1 ; CN(CC(c1ccccc1)n1cc(N)cn1)S(C)(=O)=O]

To a solution of N-phenacylmethanesulfonamide (313 mg, 1.47 mmol, see WO2007/69977 A1) in acetone (3 mL) was added iodomethane (3 equiv., 4.40 mmol, 628 mg, 0.275 mL) and potassium carbonate (1.00 equiv., 1.47 mmol, 203 mg) and the mixture was heated to 110° C. (sealed tube) overnight. The mixture was concentrated in vacuo, then purification by CombiFlash (12 g; dry load; 100:0 to 50:50 heptane:EtOAc over 16 minutes) provided N-methyl-N-phenacylmethanesulfonamide (113 mg, 0.497 mmol, 33% yield).

The title compound was then prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with N-methyl-N-phenacylmethanesulfonamide.

### Example A14

### 1-(2-(methylthio)-1-phenylethyl)-1H-pyrazol-4-amine

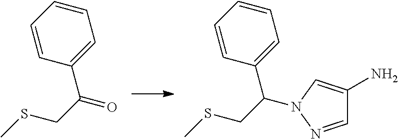 [SMILES: CSCC(=O)c1ccccc1 ; CSCC(c1ccccc1)n1cc(N)cn1]

Prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with 2-(methylthio)-1-phenylethanone (commercial).

### Example A15

### (2S)-tert-butyl 2-((4-amino-1H-pyrazol-1-yl)(phenyl)methyl)morpholine-4-carboxylate

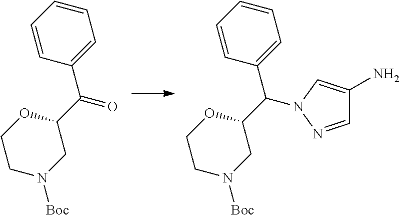 [SMILES: CC(C)(C)OC(=O)N1CCO[C@H](C(=O)c2ccccc2)C1 ; CC(C)(C)OC(=O)N1CCO[C@H](C(c2ccccc2)n2cc(N)cn2)C1]

Prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with (S)-tert-butyl 2-benzoylmorpholine-4-carboxylate (Bioorg. Med. Chem. Lett. 2008, 18, 2562). The product is obtained as an unassigned 2:1 mixture of diastereomers.

### Example A16

### 1-(pyridin-3-yl(tetrahydro-2H-thiopyran-4-yl)methyl)-1H-pyrazol-4-amine

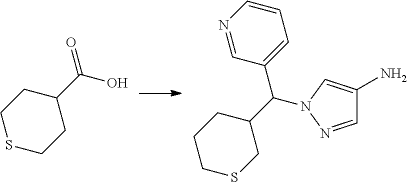 [SMILES: O=C(O)C1CCSCC1 ; Nc1cnn(C(c2cccnc2)C2CCCSC2)c1]

To a solution of tetrahydrothiopyran-4-carboxylic acid (517 mg, 3.54 mmol, 517 mg) in N,N-dimethylformamide (15 mL) was added N,O-dimethylhydroxylamine hydrochloride (1.3 equiv., 4.60 mmol, 458 mg), HATU (1.3 equiv., 4.60 mmol, 1747 mg) and N,N-diisopropylethylamine (3 equiv., 10.6 mmol, 1371 mg, 1.8 mL). The mixture was stirred overnight at rt, then diluted with 100 mL Et₂O and washed with 100 mL sat. NaHCO₃(aq) and 2×100 mL 1:1 H₂O:brine. The organic extracts were dried (Na₂SO₄ added) and concentrated in vacuo to provide N-methoxy-N-methyl-tetrahydrothiopyran-4-carboxamide (472 mg, 2.49 mmol, 70% yield).

A solution of 3-bromopyridine (1.5 equiv., 3.74 mmol, 597 mg, 0.364 mL) in diethyl ether (10 mL) was cooled to −78° C. and butyllithium (1.6 mol/L) in hexanes (1.4 equiv., 3.49 mmol, 1500 mg, 2.2 mL) was added dropwise. After stirring for 30 minutes (thick precipitate had formed), a solution of N-methoxy-N-methyl-tetrahydrothiopyran-4-carboxamide (472 mg, 2.4937 mmol) in 4 mL Et₂O was added. This mixture was stirred for 2 hours at −78° C., then overnight at rt. The reaction was quenched by the addition of ˜15 mL sat. NH₄Cl(aq) then the mixture was diluted with 50 mL EtOAc and washed with 50 mL brine. The organic extracts were dried (Na₂SO₄) and concentrated in vacuo. Purification by CombiFlash (24 g; dry load; 70:30 to 20:80 heptane:EtOAc over 20 minutes) provided 3-pyridyl(tetrahydrothiopyran-4-yl)methanone (443 mg, 2.137 mmol, 85.70% yield, 443 mg).

The title compound was then prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with 3-pyridyl(tetrahydrothiopyran-4-yl)methanone.

### Example A17

### ethyl 2-(4-amino-1H-pyrazol-1-yl)-2-(pyridin-3-yl)acetate

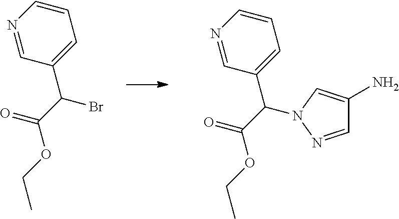 [SMILES: CCOC(=O)C(Br)c1cccnc1 ; CCOC(=O)C(c1cccnc1)n1cc(N)cn1]

Prepared in an analogous manner to methyl 2-(4-amino-1H-pyrazol-1-yl)-2-phenylacetate (Example A11), replacing ethyl 2-chloro-2-phenyl-acetate with ethyl 2-bromo-2-(pyridin-3-yl)acetate (see J. Am. Chem. Soc. 2011, 133, 16605).

### Example A18

### 1-((tetrahydro-2H-thiopyran-4-yl)(thiazol-2-yl)methyl)-1H-pyrazol-4-amine

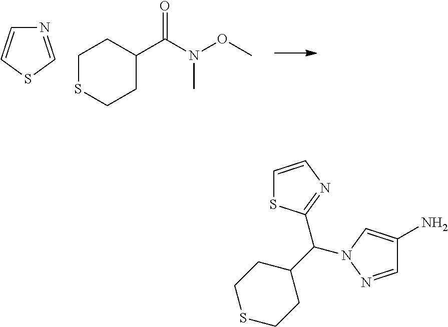

A solution of thiazole (1.5 equiv., 4.03 mmol, 347 mg, 0.289 mL) in tetrahydrofuran (10 mL, 10 mL) was cooled to −78° C., then n-butyllithium (1.6 mol/L) in hexane (1.3 equiv., 3.50 mmol, 2.2 mL) was added dropwise. The mixture was stirred for 30 minutes at this temperature, then 30 minutes at −10° C., resulting in formation of deep brown solution. N-methoxy-N-methyl-tetrahydrothiopyran-4-carboxamide (509 mg, 2.69 mmol, see Example A16) was then added dropwise as a solution in ˜1 mL THF, then the mixture was stirred for 90 minutes while warming to rt. The reaction was quenched by the addition of 10 mL sat. NH₄Cl(aq), then the mixture was diluted with 50 mL brine and extracted with 50 mL EtOAc. The organic extracts were dried (Na₂SO₄) and concentrated in vacuo. Purification by CombiFlash (24 g; dry load; 60:40 to 20:80 heptane:EtOAc over 15 minutes) provided tetrahydrothiopyran-4-yl(thiazol-2-yl)methanone (499 mg, 2.339 mmol, 87.00% yield, 499 mg).

The title compound was then prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with tetrahydrothiopyran-4-yl(thiazol-2-yl)methanone.

### Example A19

### 1-(pyridin-3-yl(tetrahydro-2H-pyran-4-yl)methyl)-1H-pyrazol-4-amine

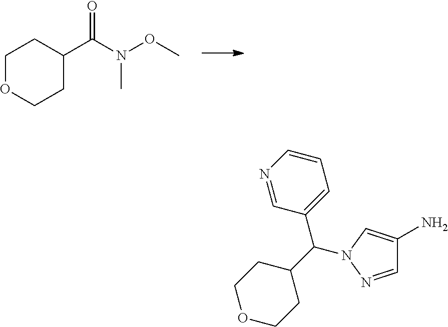 [SMILES: CON(C)C(=O)C1CCOCC1 ; Nc1cnn(C(c2cccnc2)C2CCOCC2)c1]

Prepared in an analogous manner to 1-(pyridin-3-yl(tetrahydro-2H-thiopyran-4-yl)methyl)-1H-pyrazol-4-amine (Example A16), replacing N-methoxy-N-methyl-tetrahydrothiopyran-4-carboxamide (second step) with N-methoxy-N-methyltetrahydro-2H-pyran-4-carboxamide (commercial).

### Example A20

### tert-butyl 3-((4-amino-1H-pyrazol-1-yl)(phenyl)methyl)azetidine-1-carboxylate

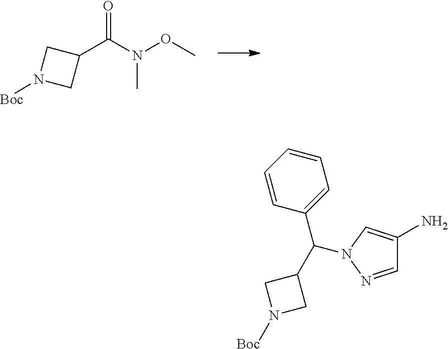 [SMILES: CON(C)C(=O)C1CN(C(=O)OC(C)(C)C)C1 ; CC(C)(C)OC(=O)N1CC(C(c2ccccc2)n2cc(N)cn2)C1]

Tert-butyl 3-[methoxy(methyl)carbamoyl]azetidine-1-carboxylate (1.11 g, 4.55 mmol, commercial) in dry tetrahydrofuran (17 mL) at 0° C. was added phenylmagnesium bromide (3.0 M in Et₂O, 2.00 equiv., 9.10 mmol, 3.03 mL) dropwise. The sample was allowed to warm slowly to room temperature and stirred overnight. The sample was diluted with sat NH₄Cl(aq), extracted 3 times with EtOAc, dried over MgSO₄, filtered, evaporated, and purified by CombiFlash (40 g, 0-30% EtOAc in heptane, 14 min gradient) to provide tert-butyl 3-benzoylazetidine-1-carboxylate (870 mg, 3.30 mmol, 73%).

The title compound was then prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) tert-butyl 3-benzoylazetidine-1-carboxylate.

### Example A21

### 1-(3-((4-amino-1H-pyrazol-1-yl)(phenyl)methyl)azetidin-1-yl)ethanone

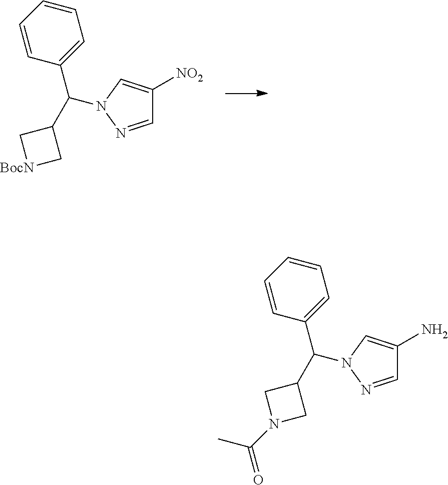 [SMILES: CC(C)(C)OC(=O)N1CC(C(c2ccccc2)n2cc([N+](=O)[O-])cn2)C1 ; CC(=O)N1CC(C(c2ccccc2)n2cc(N)cn2)C1]

Tert-butyl 3-[(4-nitropyrazol-1-yl)-phenyl-methyl]azetidine-1-carboxylate (0.53 g, 1.5 mmol, penultimate intermediate en route to Example A20) and trifluoroacetic acid (6 mL) were combined and stirred for 1 hour. The sample was evaporated, diluted with sat NaHCO₃(aq) and 10% MeOH in dichloromethane, extracted 5 times with 10% MeOH in dichloromethane, dried over MgSO₄, filtered, and evaporated to provide 1-[azetidin-3-yl(phenyl)methyl]-4-nitro-pyrazole (340 mg, 1.30 mmol, 89% yield).

1-[Azetidin-3-yl(phenyl)methyl]-4-nitro-pyrazole (0.182 g, 0.703 mmol) in dry dichloromethane (4 mL) was added N,N-diisopropylethylamine (2.00 equiv., 1.41 mmol, 0.25 mL) followed by acetyl chloride (1.20 equiv., 0.844 mmol, 0.061 mL). The sample was stirred for 30 min, then was evaporated and purified by CombiFlash (12 g, 0-10% MeOH in dichloromethane, 11 min gradient) to provide 1-[3-[(4-nitropyrazol-1-yl)-phenyl-methyl]azetidin-1-yl]ethanone (0.189 g, 0.63 mmol, 89% yield).

The title compound was then obtained by palladium on carbon mediated reduction of the nitropyrazole as outlined in Example A3.

### Example A22

### 1-((1-methylazetidin-3-yl)(phenyl)methyl)-1H-pyrazol-4-amine

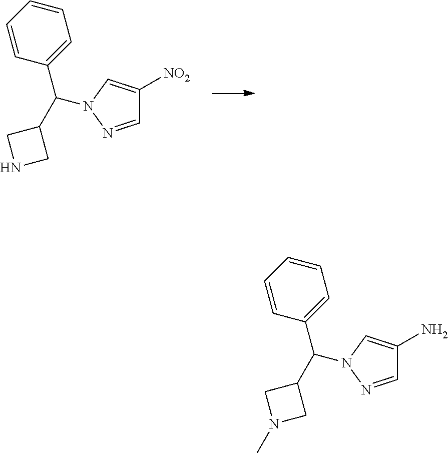 [SMILES: O=[N+]([O-])c1cnn(C(c2ccccc2)C2CNC2)c1 ; CN1CC(C(c2ccccc2)n2cc(N)cn2)C1]

1-[Azetidin-3-yl(phenyl)methyl]-4-nitro-pyrazole (0.164 g, 0.636 mmol, see Example A21) in dry N,N-dimethylformamide (3 mL) at 0° C. was added 37% aqueous formaldehyde (2.00 equiv., 1.272 mmol, 0.095 mL). The sample was then heated at 50° C. for 10 min. The sample was then cooled to 0° C. and then added sodium triacetoxyborohydride (2.50 equiv., 1.59 mmol, 337 mg). The sample was allowed to warm slowly to room temperature and stirred overnight. The sample was diluted with H₂O, extracted 8 times with 10% MeOH in dichloromethane, dried over MgSO4, filtered, evaporated, and purified by CombiFlash (12 g, 0-10% MeOH in dichloromethane, 11 min gradient) to provide 1-[(1-methylazetidin-3-yl)-phenyl-methyl]-4-nitro-pyrazole (173 mg, 0.463 mmol, 73% yield).

The title compound was then obtained by palladium on carbon mediated reduction of the nitropyrazole as outlined in Example A3.

### Example A23

### tert-butyl (3-(4-amino-1-pyrazol-1-yl)-3-phenylpropyl)carbamate

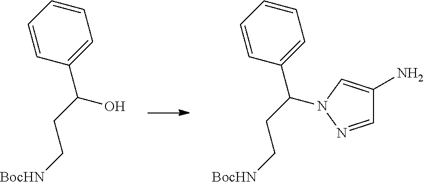 [SMILES: CC(C)(C)OC(=O)NCCC(O)c1ccccc1 ; CC(C)(C)OC(=O)NCCC(c1ccccc1)n1cc(N)cn1]

Prepared in an analogous manner to 1-(3-(dimethylamino)-1-phenylpropyl)-1H-pyrazol-4-amine (Example A3), replacing 3-(dimethylamino)-1-phenyl-propan-1-ol with tert-butyl (3-hydroxy-3-phenylpropyl)carbamate (see WO2006/113837 A2).

### Example A24

### tert-butyl 4-((4-amino-1H-pyrazol-1-yl)(phenyl)methyl)piperidine-1-carboxylate

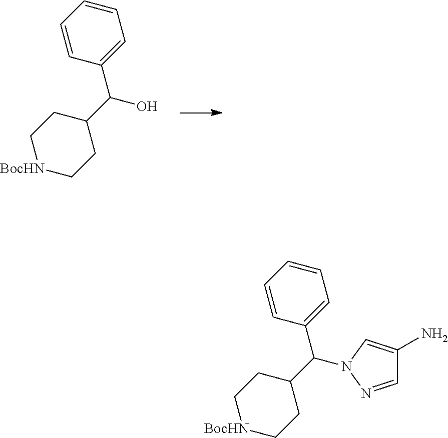

Prepared in an analogous manner to 1-(3-(dimethylamino)-1-phenylpropyl)-1H-pyrazol-4-amine (Example A3), replacing 3-(dimethylamino)-1-phenyl-propan-1-ol with tert-butyl 4-(hydroxy(phenyl)methyl)piperidine-1-carboxylate (see J. Med. Chem. 2003, 46, 5512).

### Example A25

### 1-((1-methylpiperidin-4-yl)(phenyl)methyl)-1H-pyrazol-4-amine

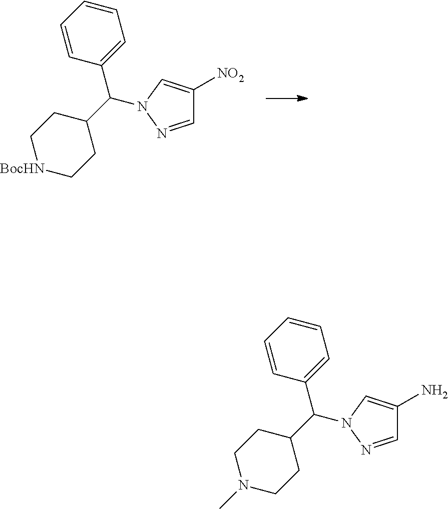

Tert-butyl 4-[(4-nitropyrazol-1-yl)-phenyl-methyl]piperidine-1-carboxylate (0.707 g, 1.83 mmol, penultimate intermediate en route to Example A24) and trifluoroacetic acid (7 mL) were combined and stirred for 1 hour. The sample was concentrated, then was diluted with sat NaHCO₃(aq), extracted 9 times with 10% MeOH in dichloromethane, dried over MgSO₄, filtered, and evaporated to provide 4-[(4-nitropyrazol-1-yl)-phenyl-methyl]piperidine (523 mg, 1.83 mmol, 100% yield).

The title compound was then obtained in an analogous manner to 1-((1-methylazetidin-3-yl)(phenyl)methyl)-1H-pyrazol-4-amine (Example A22), replacing 1-[azetidin-3-yl(phenyl)methyl]-4-nitro-pyrazole with 4-[(4-nitropyrazol-1-yl)-phenyl-methyl]piperidine.

### Example A26

### 1-(2-(methylthio)-1-(pyridin-3-yl)ethyl)-1H-pyrazol-4-amine

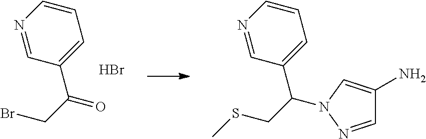 [SMILES: O=C(CBr)c1cccnc1 ; Br ; CSCC(c1cccnc1)n1cc(N)cn1]

2-Bromo-1-(3-pyridyl)ethanone hydrobromide (2.00 g, 7.12 mmol) in dry methanol (6 mL) was added sodium thiomethoxide (1.40 equiv., 9.97 mmol, 699 mg) followed by N,N′-diisopropylethylamine (2.00 equiv., 14.2 mmol, 2.51 mL). The sample was stirred for 1 hour then was diluted with H₂O, extracted 3 times with CH₂Cl₂, dried over MgSO₄, filtered, and concentrated, Purification by CombiFlash (40 g, 0-100% EtOAc in heptane, 14 min gradient) provided 2-methylsulfanyl-1-(3-pyridyl)ethanone (1.04 g, 6.24 mmol, 88% yield).

The title compound was then prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with 2-methylsulfanyl-1-(3-pyridyl)ethanone.

### Example A27

### 1-(3-(3,3-difluoroazetidin-1-yl)-1-phenylpropyl)-1H-pyrazol-4-amine

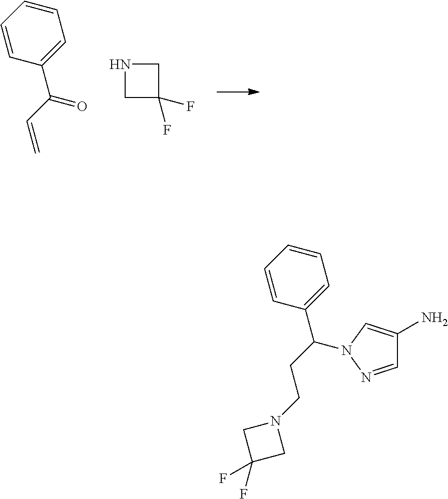

A solution of 3,3-difluoroazetidine (465 mg, 4.99 mmol) in methanol (4 mL) was added 1-phenylprop-2-en-1-one (660 mg, 4.99 mmol). The reaction mixture was stirred at rt for 2 hours then concentrated to dryness. Purification by CombiFlash (24 g; 0-20% CH₂Cl₂ in MeOH) provided 3-(3,3-difluoroazetidin-1-yl)-1-phenyl-propan-1-one (270 mg, 1.19 mmol, 24% yield).

The title compound was then prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with 3-(3,3-difluoroazetidin-1-yl)-1-phenyl-propan-1-one.

### Example A28

### 1-(3-(methyl(oxetan-3-yl)amino)-1-phenylpropyl)-1H-pyrazol-4-amine

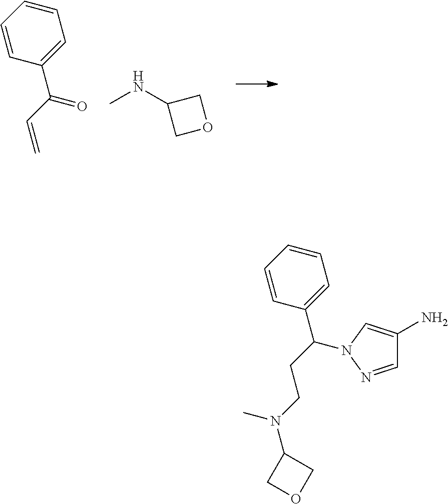

Prepared in an analogous manner to 1-(3-(3,3-difluoroazetidin-1-yl)-1-phenylpropyl)-1H-pyrazol-4-amine (Example A27), replacing 3,3-difluoroazetidine with N-methyloxetan-3-amine.

### Example A29

### 1-(2-morpholino-1-phenylethyl)-1H-pyrazol-4-amine

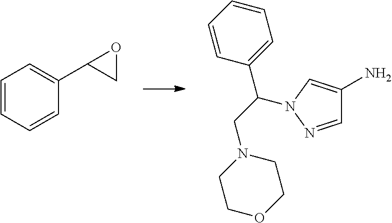 [SMILES: c1ccc(C2CO2)cc1 ; Nc1cnn(C(CN2CCOCC2)c2ccccc2)c1]

Prepared in an analogous manner to 1-(2-(dimethylamino)-1-phenylethyl)-1H-pyrazol-4-amine (Example A5), replacing dimethylamine hydrochloride with morpholine.

### Example A30

### 1-(1-(tetrahydro-2H-pyran-4-yl)ethyl)-1H-pyrazol-4-amine

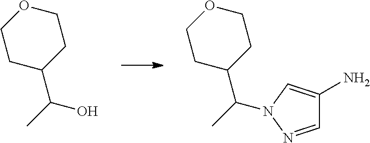

Prepared in an analogous manner to 1-(3-(dimethylamino)-1-phenylpropyl)-1H-pyrazol-4-amine (Example A3), replacing 3-(dimethylamino)-1-phenyl-propan-1-ol with 1-tetrahydropyran-4-ylethanol (see ChemMedChem 2010, 5, 65).

### Example A31

### 1-(2-(dimethylamino)ethyl)-1H-pyrazol-4-amine

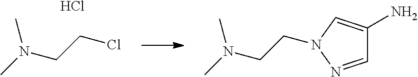

Prepared in an analogous manner to 3-((4-amino-1H-pyrazol-1-yl)methyl)benzonitrile (Example A1), replacing m-cyanobenzyl bromide with 2-chloro-N,N-dimethylethanamine hydrochloride.

### Example A32

### 4-(4-amino-1H-pyrazol-1-yl)-4-(pyridin-3-yl)butanenitrile

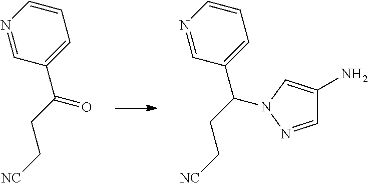 [SMILES: N#CCCC(=O)c1cccnc1 ; N#CCCC(c1cccnc1)n1cc(N)cn1]

Prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with 4-oxo-4-(pyridin-3-yl)butanenitrile (commercial).

### Example A33

### 1-(1-(thiazol-4-yl)ethyl)-1H-pyrazol-4-amine

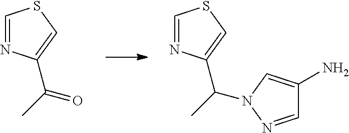

Prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with 1-(thiazol-4-yl)ethanone (commercial).

### Example A34

### 1-(phenyl(tetrahydro-2H-pyran-4-yl)methyl)-1H-pyrazol-4-amine

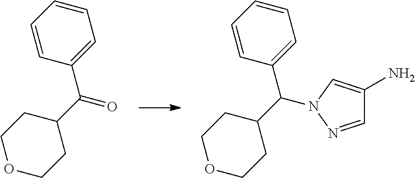 [SMILES: O=C(c1ccccc1)C1CCOCC1 ; Nc1cnn(C(c2ccccc2)C2CCOCC2)c1]

Prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with phenyl(tetrahydro-2H-pyran-4-yl)methanone (see J. Med. Chem. 2003, 46, 5512).

### Example A35

### 1-((1-methyl-1H-imidazol-2-yl)(thiophen-2-yl)methyl)-1H-pyrazol-4-amine

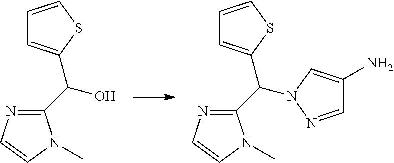 [SMILES: Cn1ccnc1C(O)c1cccs1 ; Cn1ccnc1C(c1cccs1)n1cc(N)cn1]

Prepared in an analogous manner to 1-(3-(dimethylamino)-1-phenylpropyl)-1H-pyrazol-4-amine (Example A3), replacing 3-(dimethylamino)-1-phenyl-propan-1-ol with (1-methyl-1H-imidazol-2-yl)(thiophen-2-yl)methanol (commercial).

### Example A36

### 1-(1-(1-methyl-1H-pyrazol-4-yl)ethyl)-1H-pyrazol-4-amine

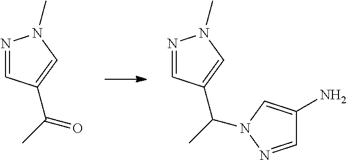

Prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with 1-(1-methyl-1H-pyrazol-4-yl)ethanone (commercial).

### Example A37

### 1-(1-(1-ethyl-1H-pyrazol-4-yl)ethyl)-1H-pyrazol-4-amine

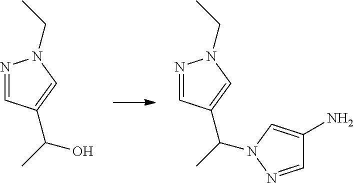

Prepared in an analogous manner to 1-(3-(dimethylamino)-1-phenylpropyl)-1H-pyrazol-4-amine (Example A3), replacing 3-(dimethylamino)-1-phenyl-propan-1-ol with 1-(1-ethyl-1H-pyrazol-4-yl)ethanol (commercial).

### Example A38

### 1-(1-(1-ethyl-1H-pyrazol-4-yl)propyl)-1H-pyrazol-4-amine

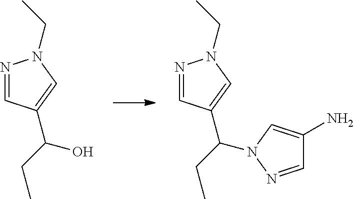 [SMILES: CCC(O)c1cnn(CC)c1 ; CCC(c1cnn(CC)c1)n1cc(N)cn1]

Prepared in an analogous manner to 1-(3-(dimethylamino)-1-phenylpropyl)-1H-pyrazol-4-amine (Example A3), replacing 3-(dimethylamino)-1-phenyl-propan-1-ol with 1-(1-ethyl-1H-pyrazol-4-yl)propan-1-ol (commercial).

### Example A39

### 1-(1-(thiazol-2-yl)ethyl)-1H-pyrazol-4-amine

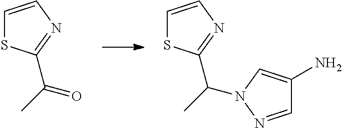

Prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with 1-(thiazol-2-yl)ethanone (commercial).

### Example A40

### 1-(1-(thiazol-5-yl)ethyl)-1H-pyrazol-4-amine

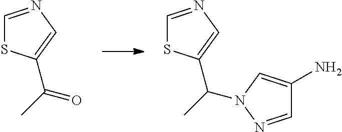

Prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with 1-(thiazol-5-yl)ethanone (commercial).

### Example A41

### 1-(1-(5-methyl-1,3,4-oxadiazol-2-yl)ethyl)-1H-pyrazol-4-amine

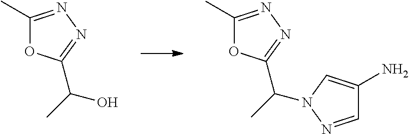

Prepared in an analogous manner to 1-(3-(dimethylamino)-1-phenylpropyl)-1H-pyrazol-4-amine (Example A3), replacing 3-(dimethylamino)-1-phenyl-propan-1-ol with 1-(5-methyl-1,3,4-oxadiazol-2-yl)ethanol (commercial).

### Example A42

### 1-(1-(oxazol-2-yl)ethyl)-1H-pyrazol-4-amine

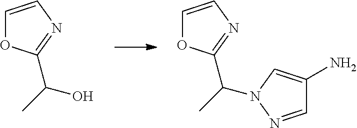

Prepared in an analogous manner to 1-(3-(dimethylamino)-1-phenylpropyl)-1H-pyrazol-4-amine (Example A3), replacing 3-(dimethylamino)-1-phenyl-propan-1-ol with 1-(oxazol-2-yl)ethanol (see WO2009/77990 A1).

### Example A43

### 1-(1-(pyridin-2-yl)ethyl)-1H-pyrazol-4-amine

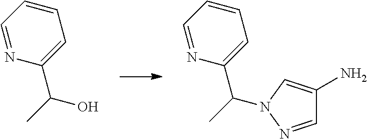

Prepared in an analogous manner to 1-(3-(dimethylamino)-1-phenylpropyl)-1H-pyrazol-4-amine (Example A3), replacing 3-(dimethylamino)-1-phenyl-propan-1-ol with 1-(pyridin-2-yl)ethanol (commercial).

### Example A44

### 1-(1-(pyridin-3-yl)ethyl)-1H-pyrazol-4-amine

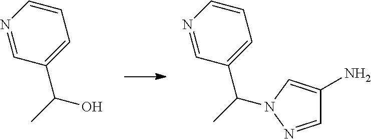

Prepared in an analogous manner to 1-(3-(dimethylamino)-1-phenylpropyl)-1H-pyrazol-4-amine (Example A3), replacing 3-(dimethylamino)-1-phenyl-propan-1-ol with 1-(pyridin-3-yl)ethanol (commercial).

### Example A45

### 1-(1-(pyridin-4-yl)ethyl)-1H-pyrazol-4-amine

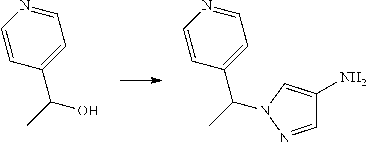

Prepared in an analogous manner to 1-(3-(dimethylamino)-1-phenylpropyl)-1H-pyrazol-4-amine (Example A3), replacing 3-(dimethylamino)-1-phenyl-propan-1-ol with 1-(pyridin-4-yl)ethanol (commercial).

### Example A46

### 1-(1-(5-fluoropyridin-3-yl)ethyl)-1H-pyrazol-4-amine

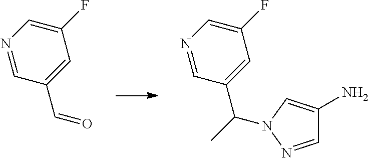

To an ice water cooled solution of methylmagnesium bromide (3.0 M in THF, 3.0 equiv, 24 mmol, 8.0 mL) in 20 mL of diethyl ether was added slowly dropwise a solution of 5-fluoropyridine-3-carbaldehyde (1.00 g, 8.00 mmol) in 10 mL of diethyl ether. The reaction mixture was stirred at cooled temp (0° C.) for 1 hour, then a saturated ammonium chloride solution was added, followed by extraction with EtOAc. The organic extracts were dried (Na₂SO₄) and concentrated in vacuo to provide 1-(5-fluoropyridin-3-yl)ethanol (800 mg, 74% yield) of sufficient purity to be used directly.

The title compound was then prepared in an analogous manner to 1-(3-(dimethylamino)-1-phenylpropyl)-1H-pyrazol-4-amine (Example A3), replacing 3-(dimethylamino)-1-phenyl-propan-1-ol with 1-(5-fluoropyridin-3-yl)ethanol.

### Example A47

### 1-(1-(6-methoxypyridin-3-yl)ethyl)-1H-pyrazol-4-amine

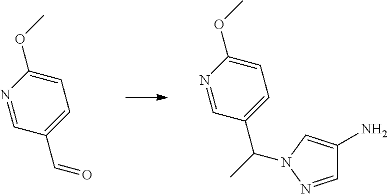

Prepared in an analogous manner to 1-(1-(5-fluoropyridin-3-yl)ethyl)-1H-pyrazol-4-amine (Example A46), replacing 5-fluoropyridine-3-carbaldehyde with 6-methoxynicotinaldehyde.

### Example A48

### 1-((3-chlorophenyl)(tetrahydro-2H-pyran-4-yl)methyl)-1H-pyrazol-4-amine

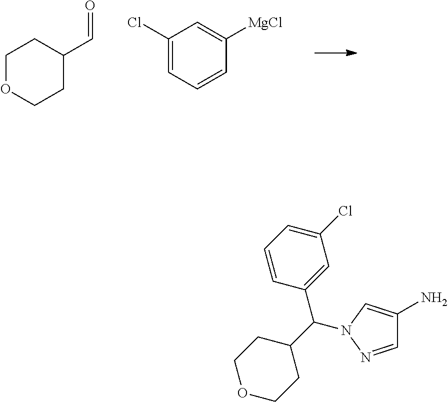

To an ice cooled solution of 3-chlorophenylmagnesium bromide (0.5 M in THF, 2.00 equiv, 4.20 mmol, 8.0 mL) was added tetrahydropyran-4-carbaldehyde (2.10 mmol, 250 mg) neat by slow dropwise addition. The reaction mixture was stirred another 30 min and then allowed to warm to room temperature overnight. The reaction mixture was cooled with ice water and quenched by the addition of sat. NH₄Cl(aq) and diluted with EtOAc. The organic extracts were washed with brine, drive (Na₂SO₄) and concentrated in vacuo. Purification by CombiFlash (40 g, 10-70% EtOAc/hep) to provide (3-chlorophenyl)(tetrahydro-2H-pyran-4-yl)methanol (380 mg, 1.67 mmol, 80% yield).

The title compound was then prepared in an analogous manner to 1-(3-(dimethylamino)-1-phenylpropyl)-1H-pyrazol-4-amine (Example A3), replacing 3-(dimethylamino)-1-phenyl-propan-1-ol with (3-chlorophenyl)(tetrahydro-2H-pyran-4-yl)methanol.

### Example A49

### 1-((1-methyl-1H-pyrazol-4-yl)(phenyl)methyl)-1H-pyrazol-4-amine

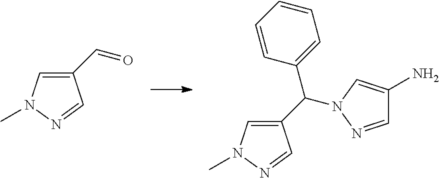

Prepared in an analogous manner to 1-((3-chlorophenyl)(tetrahydro-2H-pyran-4-yl)methyl)-1H-pyrazol-4-amine amine (Example A48), replacing tetrahydropyran-4-carbaldehyde with 1-methyl-1H-pyrazole-4-carbaldehyde and replacing 3-chlorophenylmagnesium bromide with phenylmagnesium bromide (1.0 M solution in THF).

### Example A50

### 1-(3-(dimethylamino)-1-(m-tolyl)propyl)-1H-pyrazol-4-amine

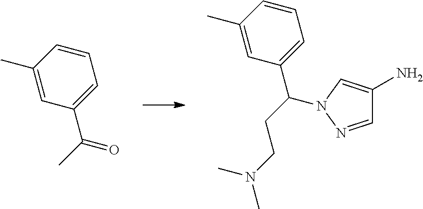

Into a 500-mL 3-necked round-bottom flask, was placed 1-(3-methylphenyl)ethan-1-one (10 g, 74.53 mmol, 1.00 equiv), formaldehyde (6.5 g, 216.48 mmol, 1.44 equiv), dimethylamine hydrochloride (10 g, 122.63 mmol, 1.65 equiv), ethanol (150 mL), hydrogen chloride (1 mL). The resulting solution was stirred at 80° C. overnight and concentrated under vacuum. The residue was mixed with ethyl acetate. The solids were filtered out and the filtrate was concentrated under vacuum. This resulted in 18 g (crude) of 3-(dimethylamino)-1-(3-methylphenyl)propan-1-one as a yellow solid.

Into a 100-mL 3-necked round-bottom flask, was placed 3-(dimethylamino)-1-(3-methylphenyl)propan-1-one (3 g, 15.68 mmol, 1.00 equiv), methanol (30 mL), NaBH₄ (1.8 g, 48.88 mmol, 3.00 equiv). The resulting solution was stirred for 1 hour at room temperature and concentrated under vacuum. The residue was mixed with ethyl acetate. The solids were filtered out and the filtrate was concentrated under vacuum. This resulted in 1.4 g (46%) of 3-(dimethylamino)-1-(3-methylphenyl)propan-1-ol as yellow oil.

The title compound was then prepared in an analogous manner to 1-(3-(dimethylamino)-1-phenylpropyl)-1H-pyrazol-4-amine (Example A3), replacing 3-(dimethylamino)-1-phenyl-propan-1-ol with 3-(dimethylamino)-1-(3-methylphenyl)propan-1-ol.

### Example A51

### 1-(1-(3-chlorophenyl)-3-(dimethylamino)propyl)-1H-pyrazol-4-amine

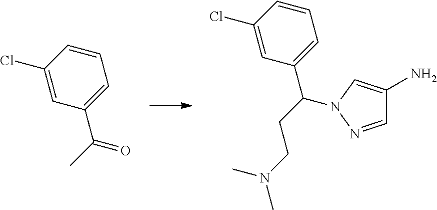

Prepared in an analogous manner to 1-(3-(dimethylamino)-1-(m-tolyl)propyl)-1H-pyrazol-4-amine (Example A50), replacing 1-(3-methylphenyl)ethan-1-one with 1-(3-chlorophenyl)ethan-1-one.

### Example A52

### 1-(3-(dimethylamino)-1-(3-(trifluoromethyl)phenyl)propyl)-1H-pyrazol-4-amine

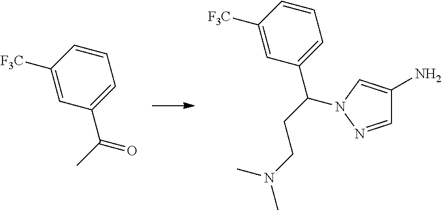 [SMILES: CC(=O)c1cccc(C(F)(F)F)c1 ; CN(C)CCC(c1cccc(C(F)(F)F)c1)n1cc(N)cn1]

Prepared in an analogous manner to 1-(3-(dimethylamino)-1-(m-tolyl)propyl)-1H-pyrazol-4-amine (Example A50), replacing 1-(3-methylphenyl)ethan-1-one with 1-(3-trifluoromethylphenyl)ethan-1-one.

### Example A53

### 1-(3-(methylsulfonyl)-1-phenylpropyl)-1H-pyrazol-4-amine

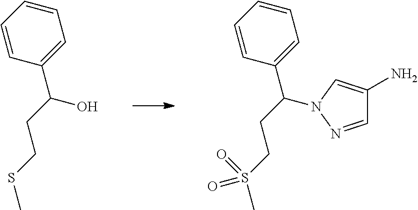 [SMILES: CSCCC(O)c1ccccc1 ; CS(=O)(=O)CCC(c1ccccc1)n1cc(N)cn1]

DIAD (83 mg, 0.41 mmol, 1.50 equiv) was added dropwise to a stirred solution of 3-(methylsulfanyl)-1-phenylpropan-1-ol (50 mg, 0.27 mmol, 1.00 equiv; see Bull. Chem. Soc. Jpn. 1977, 50, 3033), 4-nitro-1H-pyrazole (46 mg, 0.41 mmol, 1.50 equiv), and PPh₃ (108 mg, 0.41 mmol, 1.50 equiv) in tetrahydrofuran (30 mL) at 0° C. under nitrogen. The resulting solution was stirred at room temperature overnight, concentrated under vacuum, and purified by silica gel chromatography with ethyl acetate/petroleum ether (1:30). This resulted in 60 mg (79%) of 1-[3-(methylsulfanyl)-1-phenylpropyl]-4-nitro-1H-pyrazole as colorless oil.

m-CPBA (5 g, 28.97 mmol, 2.50 equiv) was added in several portions to a stirred solution of 1-[3-(methylsulfanyl)-1-phenylpropyl]-4-nitro-1H-pyrazole (3 g, 10.82 mmol, 1.00 equiv) in dichloromethane (100 mL) at room temperature. The solids from the reaction were filtered out and the filtrate was diluted with 300 mL of dichloromethane. The resulting mixture was washed with 2×150 mL of saturated sodium carbonate, dried over anhydrous sodium sulfate, and concentrated under vacuum. This resulted in 3.6 g (crude) of 1-(3-methanesulfonyl-1-phenylpropyl)-4-nitro-1H-pyrazole as yellow oil.

Into a 250-mL round-bottom flask purged and maintained with hydrogen atmosphere was placed a mixture of 1-(3-methanesulfonyl-1-phenylpropyl)-4-nitro-1H-pyrazole (1 g, 3.23 mmol, 1.00 equiv) and Raney-Ni (1 g) in methanol (100 mL). The resulting solution was stirred for 5 h at room temperature under hydrogen atmosphere. The solids were filtered out and the filtrate was concentrated under vacuum. This resulted in 960 mg (crude) of 1-(3-(methylsulfonyl)-1-phenylpropyl)-1H-pyrazol-4-amine as brown oil.

### Example A54

### tert-butyl 3-((4-amino-1H-pyrazol-1-yl)(m-tolyl)methyl)azetidine-1-carboxylate

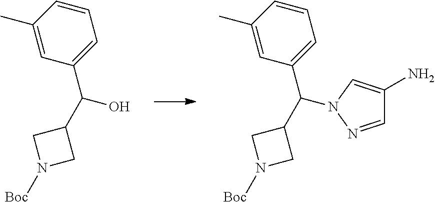 [SMILES: Cc1cccc(C(O)C2CN(C(=O)OC(C)(C)C)C2)c1 ; Cc1cccc(C(C2CN(C(=O)OC(C)(C)C)C2)n2cc(N)cn2)c1]

Prepared in an analogous manner to 1-((3-chlorophenyl)(tetrahydro-2H-pyran-4-yl)methyl)-1H-pyrazol-4-amine amine (Example A20), replacing phenylmagnesium bromide with (3-methylphenyl)magnesium bromide.

### Example A55

### 4-((4-amino-1H-pyrazol-1-yl)(phenyl)methyl)tetrahydro-2H-thiopyran 1,1-dioxide

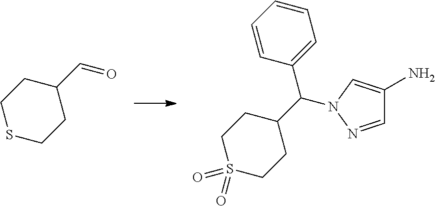 [SMILES: O=CC1CCSCC1 ; Nc1cnn(C(c2ccccc2)C2CCS(=O)(=O)CC2)c1]

Into a 2-L 3-necked round-bottom flask purged and maintained with a nitrogen atmosphere, was placed tetrahydro-2H-thiopyran-4-carbaldehyde (65 g, 499.20 mmol, 1.00 equiv) and tetrahydrofuran (300 mL). PhMgBr (1M, 750 mL, 1.50 equiv) was added dropwise to the stirred solution at 0° C. The resulting solution was then stirred for 12 h at room temperature. The reaction progress was monitored by TLC with PE/DCM=2/1. The reaction was then quenched by the addition of 500 mL of saturated NH₄Cl and extracted with 3×500 mL of ethyl acetate. The organic was washed with 3×500 mL of brine, dried over anhydrous sodium sulfate and concentrated under vacuum. The residue was applied onto a silica gel column with ethyl acetate/petroleum ether (1/20). This resulted in 27.0 g (26%) of phenyl(tetrahydro-2H-thiopyran-4-yl)methanol as a yellow oil.

The title compound was then prepared in an analogous manner to 1-(3-(methylsulfonyl)-1-phenylpropyl)-1H-pyrazol-4-amine (Example A53), replacing 3-(methylsulfanyl)-1-phenylpropan-1-ol with phenyl(tetrahydro-2H-thiopyran-4-yl).

### Example A56

### 1-((1-(2-fluoroethyl)azetidin-3-yl)(phenyl)methyl)-1H-pyrazol-4-amine

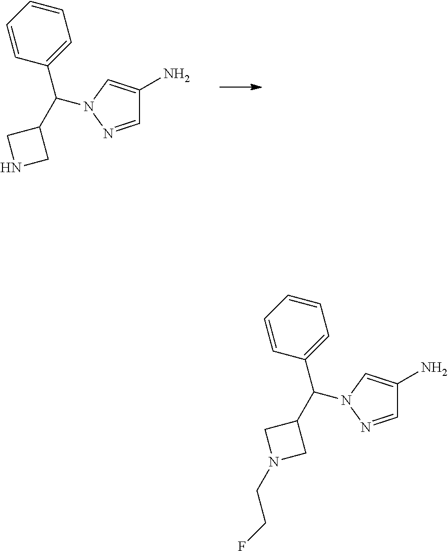 [SMILES: Nc1cnn(C(c2ccccc2)C2CNC2)c1 ; Nc1cnn(C(c2ccccc2)C2CN(CCF)C2)c1]

A mixture of 1-[azetidin-3-yl(phenyl)methyl]-4-nitro-pyrazole (3 g, 11.62 mmol, 1.00 equiv, see Example A21), 1-bromo-2-fluoroethane (2.2 g, 17.33 mmol, 1.50 equiv), and potassium carbonate (3.2 g, 23.15 mmol, 2.00 equiv) in N,N-dimethylformamide (50 mL) was stirred at room temperature overnight. The resulting solution was diluted with 300 mL of ethyl acetate and washed with 3×150 mL of brine. The organic was dried over sodium sulfate and concentrated under vacuum. The residue was applied onto a silica gel column with methanol/CH₂Cl₂ (1:150). This resulted in 700 mg (20%) of 1-[[1-(2-fluoroethyl)azetidin-3-yl](phenyl)methyl]-4-nitro-1H-pyrazole as a yellow syrup.

A mixture of 1-[[1-(2-fluoroethyl)azetidin-3-yl](phenyl)methyl]-4-nitro-1H-pyrazole (700 mg, 2.30 mmol, 1.00 equiv) and Raney-Ni (2 g) in methanol (50 mL) was stirred for 2 h at room temperature under hydrogen. The solids were filtered out. The filtrate was concentrated to give 600 mg (95%) of the title compound as a brown syrup.

### Example A57

### 1-((1-(2-fluoroethyl)piperidin-4-yl)(phenyl)methyl)-1H-pyrazol-4-amine

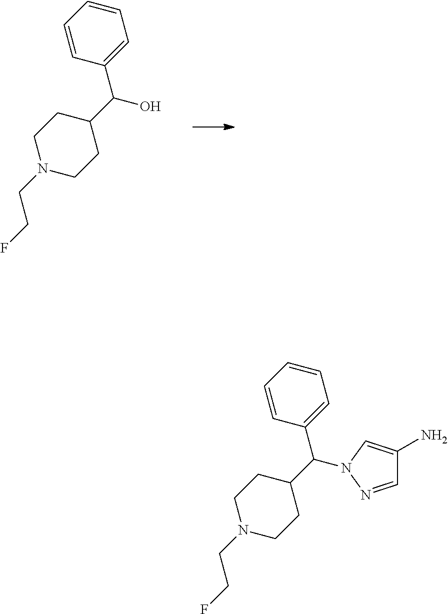 [SMILES: OC(c1ccccc1)C1CCN(CCF)CC1 ; Nc1cnn(C(c2ccccc2)C2CCN(CCF)CC2)c1]

Prepared in an analogous manner to 1-((1-(2-fluoroethyl)azetidin-3-yl)(phenyl)methyl)-1H-pyrazol-4-amine (Example A56), replacing 1-[azetidin-3-yl(phenyl)methyl]-4-nitro-pyrazole with 4-[(4-nitropyrazol-1-yl)-phenyl-methyl]piperidine (see Example A25).

### Example A58

### 1-(2-(4-amino-1H-pyrazol-1-yl)-2-phenylethyl)pyrrolidin-2-one

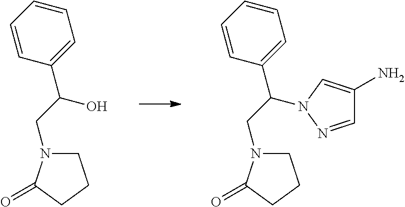 [SMILES: O=C1CCCN1CC(O)c1ccccc1 ; Nc1cnn(C(CN2CCCC2=O)c2ccccc2)c1]

Thionyl chloride (15.22 g, 127.93 mmol, 4.97 equiv) was added dropwise to a stirred solution of 1-(2-hydroxy-2-phenylethyl)pyrrolidin-2-one (5.28 g, 25.72 mmol, 1.00 equiv) (Chem. Pharm Bull. 1978, 26, 2071) in dichloromethane (150 mL) at 0° C. After 3 h at room temperature the resulting mixture was concentrated under vacuum. This resulted in 7 g (crude) of 1-(2-chloro-2-phenylethyl)pyrrolidin-2-one as a yellow syrup.

A mixture of 4-nitro-1H-pyrazole (5 g, 44.22 mmol, 1.41 equiv) and potassium carbonate (5.6 g, 40.52 mmol, 1.29 equiv) in N,N-dimethylformamide (130 mL) was stirred for 30 min at 60° C. A solution of 1-(2-chloro-2-phenylethyl)pyrrolidin-2-one (7 g, 31.29 mmol, 1.00 equiv) in N,N-dimethylformamide (30 mL) was added slowly. The resulting mixture was then stirred at 60° C. overnight. After cooling to room temperature the reaction mixture was diluted with 500 mL of ethyl acetate and washed with 3×100 mL of brine. The organic was dried over anhydrous sodium sulfate and concentrated under vacuum. The residue was applied onto a silica gel column with ethyl acetate/petroleum ether (1:1). This resulted in 7 g (74%) of 1-[2-(4-nitro-1H-pyrazol-1-yl)-2-phenylethyl]pyrrolidin-2-one as a light yellow solid.

Hydrogen gas was introduced into a mixture of 1-[2-(4-nitro-1H-pyrazol-1-yl)-2-phenylethyl]pyrrolidin-2-one (500 mg, 1.66 mmol, 1.00 equiv) and Raney-Ni (100 mg) in methanol (30 mL). The resulting mixture was stirred for 1 h at room temperature. The solids were filtered out and the filtrate was concentrated under vacuum. This resulted in 300 mg (67%) of the title compound as an off-white syrup.

### Example A59

### 4-(2-(4-amino-1H-pyrazol-1-yl)-2-phenylethyl)morpholin-3-one

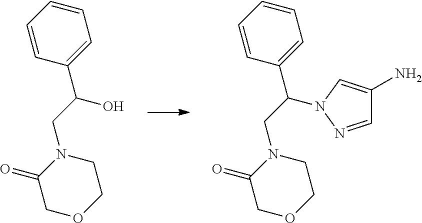 [SMILES: O=C1COCCN1CC(O)c1ccccc1 ; Nc1cnn(C(CN2CCOCC2=O)c2ccccc2)c1]

Prepared in an analogous manner to 1-(2-(4-amino-1H-pyrazol-1-yl)-2-phenylethyl)pyrrolidin-2-one (Example A58), replacing 1-(2-hydroxy-2-phenylethyl)pyrrolidin-2-one with 4-(2-hydroxy-2-phenylethyl)morpholin-3-one.

### Example A60

### 3-((4-amino-1H-pyrazol-1-yl)(phenyl)methyl)thietane 1,1-dioxide

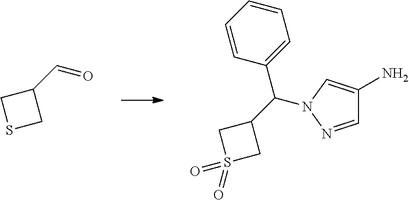 [SMILES: O=CC1CSC1 ; Nc1cnn(C(c2ccccc2)C2CS(=O)(=O)C2)c1]

Prepared in an analogous manner to 4-((4-amino-1H-pyrazol-1-yl)(phenyl)methyl)tetrahydro-2H-thiopyran 1,1-dioxide (Example A55), replacing tetrahydro-2H-thiopyran-4-carbaldehyde with thietane-3-carbaldehyde (J. Org. Chem. 1983, 48, 4852).

### Example A61

### 3-((4-amino-1H-pyrazol-1-yl)(1,1-dioxidotetrahydro-2H-thiopyran-4-yl)methyl)benzonitrile

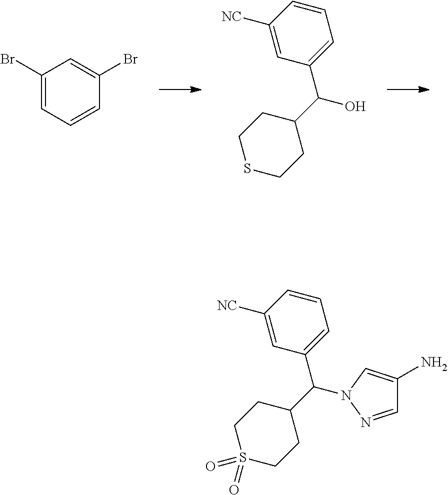 [SMILES: Brc1cccc(Br)c1 ; N#Cc1cccc(C(O)C2CCSCC2)c1 ; N#Cc1cccc(C(C2CCS(=O)(=O)CC2)n2cc(N)cn2)c1]

n-BuLi (28 mL, 2.5 M in hexanes, 1.80 equiv) was added dropwise to a stirred solution of 1,3-dibromobenzene (16.5 g, 69.94 mmol, 1.78 equiv) in tetrahydrofuran (100 mL) at −78° C. under nitrogen. After 30 min at −78° C. a solution of thiane-4-carbaldehyde (5.13 g, 39.40 mmol, 1.00 equiv) in tetrahydrofuran (10 mL) was added slowly. The resulting solution was allowed to warm to room temperature and stirred for additional 12 h. The reaction was then quenched by saturated NH₄Cl, extracted with 3×100 mL of ethyl acetate, dried over anhydrous sodium sulfate, and concentrated under vacuum. The residue was applied onto a silica gel column with ethyl acetate/petroleum ether (1/20). This resulted in 5.4 g (48%) of (3-bromophenyl)(thian-4-yl)methanol as light yellow oil.

Into a 8 mL sealed tube purged and maintained with an inert atmosphere of nitrogen was placed a solution of (3-bromophenyl)(thian-4-yl)methanol (100 mg, 0.35 mmol, 1.00 equiv) in DMSO (4 mL), zincdicarbonitrile (81 mg, 0.69 mmol, 2.00 equiv), Pd(PPh3)4 (41 mg, 0.04 mmol, 0.10 equiv). The resulting solution was stirred overnight at 80° C. The reaction was then quenched with water, extracted with 3×50 mL of ethyl acetate, and concentrated under vacuum. The residue was applied onto a silica gel column with ethyl acetate/petroleum ether (1:50-1:5). This resulted in 50 mg (62%) of 3-[hydroxy(thian-4-yl)methyl]benzonitrile as light brown oil.

The title compound was then prepared in an analogous manner to 1-(3-(methylsulfonyl)-1-phenylpropyl)-1H-pyrazol-4-amine (Example A53), replacing 3-(methylsulfanyl)-1-phenylpropan-1-ol with 3-[hydroxy(thian-4-yl)methyl]benzonitrile.

### Example A62

### 4-((4-amino-1H-pyrazol-1-yl)methyl)pyridin-2-amine

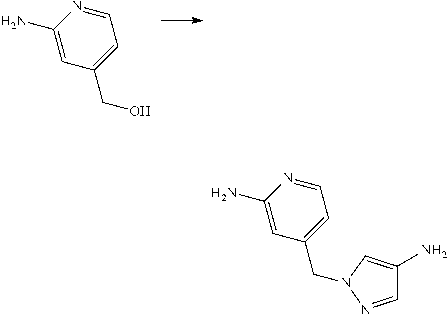 [SMILES: Nc1cc(CO)ccn1 ; Nc1cnn(Cc2ccnc(N)c2)c1]

Prepared in an analogous manner 1-(3-(dimethylamino)-1-phenylpropyl)-1H-pyrazol-4-amine (Example A3), replacing 3-(dimethylamino)-1-phenyl-propan-1-ol with (2-aminopyridin-4-yl)methanol (commercial).

### Example A63

### 3-((4-amino-1H-pyrazol-1-yl)methyl)pyridin-2-amine

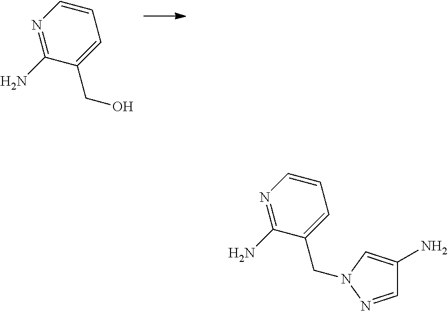 [SMILES: Nc1ncccc1CO ; Nc1cnn(Cc2cccnc2N)c1]

Prepared in an analogous manner 1-(3-(dimethylamino)-1-phenylpropyl)-1H-pyrazol-4-amine (Example A3), replacing 3-(dimethylamino)-1-phenyl-propan-1-ol with (2-aminopyridin-3-yl)methanol (commercial).

### Example A64

### 3-((4-amino-1H-pyrazol-1-yl)(pyridin-3-yl)methyl)thietane 1,1-dioxide

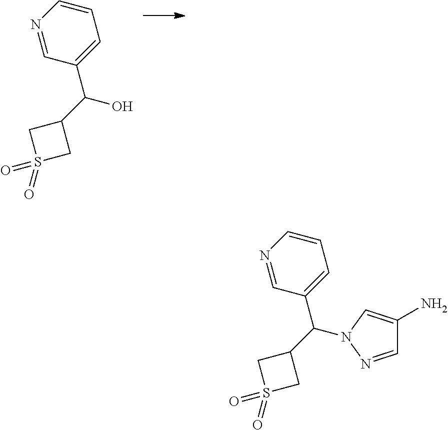 [SMILES: O=S1(=O)CC(C(O)c2cccnc2)C1 ; Nc1cnn(C(c2cccnc2)C2CS(=O)(=O)C2)c1]

Prepared in an analogous manner to 4-((4-amino-1H-pyrazol-1-yl)(phenyl)methyl)tetrahydro-2H-thiopyran 1,1-dioxide (Example A55), replacing tetrahydro-2H-thiopyran-4-carbaldehyde with thietane-3-carbaldehyde (J. Org. Chem. 1983, 48, 4852), and replacing phenylmagnesium bromide with pyridin-3-yllithium (generated in situ by adding butyl lithium to 3-bromopyridine at −90° C.).

### Example A65

### 4-((4-amino-1H-pyrazol-1-yl)(3-(2-hydroxypropan-2-yl)phenyl)methyl)tetrahydro-2H-thiopyran 1,1-dioxide

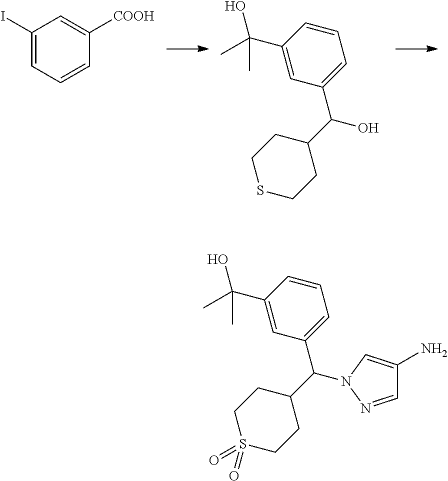 [SMILES: O=C(O)c1cccc(I)c1 ; CC(C)(O)c1cccc(C(O)C2CCSCC2)c1 ; CC(C)(O)c1cccc(C(C2CCS(=O)(=O)CC2)n2cc(N)cn2)c1]

n-BuLi (16.1 mL, 2.20 equiv) was added dropwise to a stirred solution of 3-iodobenzoic acid (5.0 g, 20.16 mmol, 1.10 equiv) in tetrahydrofuran (50 mL) under nitrogen at −78° C. After 1 h at −78° C. thiane-4-carbaldehyde (2.4 g, 18.43 mmol, 1.00 equiv) was added dropwise. The resulting solution was stirred for 2 h at −78° C., allowed to warm to room temperature, and stirred for another 10 hours at room temperature. The reaction was quenched with water and extracted with 2×30 mL of ethyl acetate. The pH value of the aqueous layer was adjusted to 4-5 with 2N hydrogen chloride. The resulting solution was extracted with 3×50 mL of ethyl acetate and the organic layers were dried over anhydrous sodium sulfate. The solvent was removed under vacuum and the product was recrystallized from ether. This resulted in 1.3 g (28%) of 3-[hydroxy(thian-4-yl)methyl]benzoic acid as a white solid.

Thionyl chloride (350 mg, 2.94 mmol, 1.00 equiv) was added to a stirred solution of 3-[hydroxy(thian-4-yl)methyl]benzoic acid (740 mg, 2.93 mmol, 1.00 equiv) in methanol (50 mL). The resulting solution was stirred at 60° C. overnight. The resulting mixture was concentrated under vacuum and the residue was mixed with 50 mL of Et₂O. The solids were filtered out and the filtrate was concentrated under vacuum. This resulted in 0.7 g (90%) of methyl 3-[hydroxy(thian-4-yl)methyl]benzoate as colorless oil.

Methyl magnesium bromide (3 M, 24 mL, 8.00 equiv) was added dropwise to a stirred solution of methyl 3-[hydroxy(thian-4-yl)methyl]benzoate (800 mg, 3.00 mmol, 1.00 equiv) in tetrahydrofuran (20 mL) under nitrogen at 0° C. The resulting solution was stirred at 25° C. overnight. The reaction mixture was diluted with 150 mL of NH₄Cl, extracted with 3×50 mL of ethyl acetate, dried over anhydrous sodium sulfate, and concentrated under vacuum. This resulted in 0.8 g (crude) of 2-[3-[hydroxy(thian-4-yl)methyl]phenyl]propan-2-ol as a white solid.

The title compound was then prepared in an analogous manner to 1-(3-(methylsulfonyl)-1-phenylpropyl)-1H-pyrazol-4-amine (Example A53), replacing 3-(methylsulfanyl)-1-phenylpropan-1-ol with of 2-[3-[hydroxy(thian-4-yl)methyl]phenyl]propan-2-ol.

### Example A66

### 4-((4-amino-1H-pyrazol-1-yl)(phenyl)methyl)tetrahydro-2H-thiopyran 1-oxide

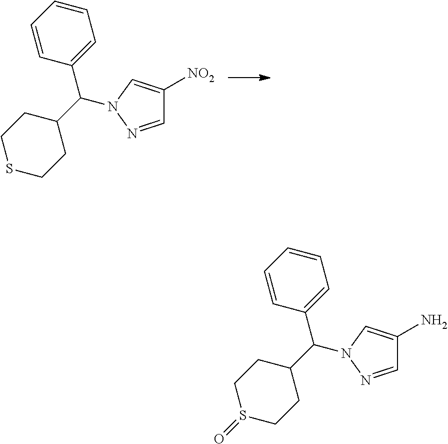 [SMILES: O=[N+]([O-])c1cnn(C(c2ccccc2)C2CCSCC2)c1 ; Nc1cnn(C(c2ccccc2)C2CCS(=O)CC2)c1]

A solution of m-CPBA (570 mg, 3.30 mmol, 1.00 equiv) in AcOEt (5 ml) was added dropwise to a stirred solution of 4-nitro-1-[phenyl(thian-4-yl)methyl]-1H-pyrazole (1.0 g, 3.30 mmol, 1.00 equiv) in dichloromethane (50 mL) at 0° C. After 30 minutes the resulting solution was diluted with 250 mL of AcOEt and washed with 3×150 mL of saturated solution of sodium carbonate and 3×150 mL of brine. The organic was dried over anhydrous sodium sulfate and concentrated under vacuum. The residue was applied onto a silica gel column with ethyl acetate/petroleum ether (1/2 to 2/1). This resulted in 500 mg (47%) and 400 mg (38%) of the two diastereomers (stereochemistry unassigned) of 4-nitro-1-[phenyl(1-oxo-thian-4-yl)methyl]-1H-pyrazole.

Each diastereomer was then reduced individually: Hydrogen gas was introduced into a mixture of 4-nitro-1-[phenyl(1-oxo-thian-4-yl)methyl]-1H-pyrazole (500 mg, 1.57 mmol, 1.00 equiv) and palladium on carbon (500 mg) in methanol (50 mL). After 30 min at room temperature the solids were filtered out. The filtrate was concentrated under vacuum. This resulted in 425 mg (94%) of 4-amino-1-[phenyl(1-oxo-thian-4-yl)methyl]-1H-pyrazole as a light yellow solid.

### Example A67

### 1-(cyclopropyl(3-(methylthio)phenyl)methyl)-1H-pyrazol-4-amine

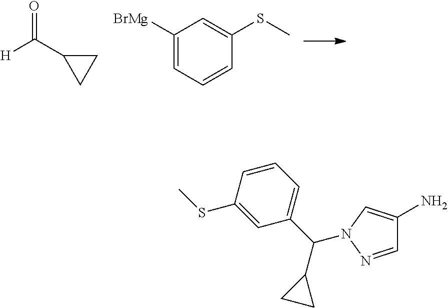

Prepared in an analogous manner to 1-((3-chlorophenyl)(tetrahydro-2H-pyran-4-yl)methyl)-1H-pyrazol-4-amine (Example A48), replacing 3-chlorophenylmagnesium bromide with (3-(methylthio)phenyl)magnesium bromide and tetrahydropyran-4-carbaldehyde with cyclopropanecarbaldehyde.

### Example A68

### 1-((3-(methylthio)phenyl)(tetrahydrofuran-3-yl)methyl)-1H-pyrazol-4-amine

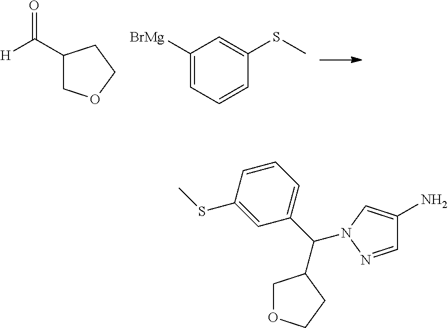

Prepared in an analogous manner to 1-((3-chlorophenyl)(tetrahydro-2H-pyran-4-yl)methyl)-1H-pyrazol-4-amine (Example A48), replacing 3-chlorophenylmagnesium bromide with (3-(methylthio)phenyl)magnesium bromide and tetrahydropyran-4-carbaldehyde with tetrahydrofuran-3-carbaldehyde.

### Example A69

### 1-((3-(methylthio)phenyl)(tetrahydro-2H-pyran-4-yl)methyl)-1H-pyrazol-4-amine

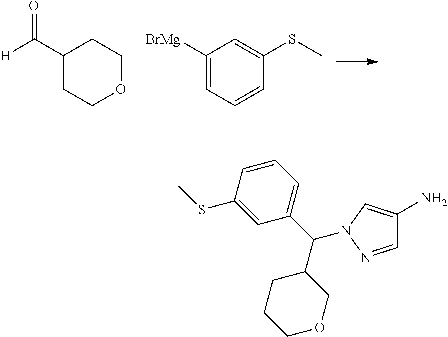

Prepared in an analogous manner to 1-((3-chlorophenyl)(tetrahydro-2H-pyran-4-yl)methyl)-1H-pyrazol-4-amine (Example A48), replacing 3-chlorophenylmagnesium bromide with (3-(methylthio)phenyl)magnesium bromide.

### Example A70

### tert-butyl 2-((4-amino-1H-pyrazol-1-yl)(phenyl)methyl)thiomorpholine-4-carboxylate

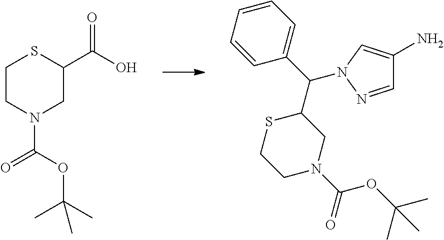 [SMILES: CC(C)(C)OC(=O)N1CCSC(C(=O)O)C1 ; CC(C)(C)OC(=O)N1CCSC(C(c2ccccc2)n2cc(N)cn2)C1]

Prepared in an analogous manner to 1-((1-(methylsulfonyl)azetidin-3-yl)(phenyl)methyl)-1H-pyrazol-4-amine (Example A7), replacing 1-methylsulfonylazetidine-3-carboxylic acid with 4-(tert-butoxycarbonyl)thiomorpholine-2-carboxylic acid (commercial).

### Example A71

### tert-butyl 3-((4-amino-1H-pyrazol-1-yl)(phenyl)methyl)thiomorpholine-4-carboxylate

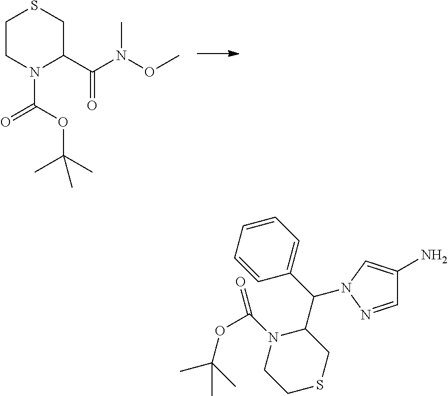 [SMILES: CON(C)C(=O)C1CSCCN1C(=O)OC(C)(C)C ; CC(C)(C)OC(=O)N1CCSCC1C(c1ccccc1)n1cc(N)cn1]

The starting material (tert-butyl 3-(methoxy(methyl)carbamoyl)thiomorpholine-4-carboxylate) was prepared in an analogous manner to the first step toward 1-((1-(methylsulfonyl)azetidin-3-yl)(phenyl)methyl)-1H-pyrazol-4-amine (Example A7), replacing 1-methylsulfonylazetidine-3-carboxylic acid with 4-(tert-butoxycarbonyl)thiomorpholine-3-carboxylic acid (commercial).

A solution of tert-butyl 3-[methoxy(methyl)carbamoyl]thiomorpholine-4-carboxylate (455 mg, 1.567 mmol, 455 mg) in tetrahydrofuran (5 mL) was cooled to 0° C., then lithium aluminum hydride (2 mol/L) in tetrahydrofuran (1.5 equiv., 2.350 mmol, 1.175 mL) was added dropwise. The mixture was stirred for 60 minutes at 0° C., then the reaction was quenched by the careful addition of water (90 μL), 15% NaOH(aq) (90 μL) and water (270 μL). The mixture was stirred for 15 minutes, then filtered through Celite (CPME rinse). After in vacuo concentration, tert-butyl 3-formylthiomorpholine-4-carboxylate was obtained in quantitative yield.

The title compound was then prepared in an analogous manner to 1-((3-chlorophenyl)(tetrahydro-2H-pyran-4-yl)methyl)-1H-pyrazol-4-amine (Example A48), replacing 3-chlorophenylmagnesium bromide with phenylmagnesium bromide and tetrahydropyran-4-carbaldehyde with tert-butyl 3-formylthiomorpholine-4-carboxylate.

### Example A72

### 1-(2-(4-amino-1H-pyrazol-1-yl)-2-phenylethyl)-3-methylimidazolidin-2-one

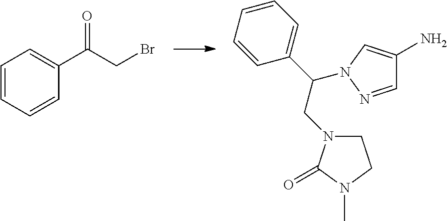 [SMILES: O=C(CBr)c1ccccc1 ; CN1CCN(CC(c2ccccc2)n2cc(N)cn2)C1=O]

A mixture of 1-methylimidazolidin-2-one (2 g, 19.98 mmol, 1.00 equiv), 2-bromo-1-phenylethan-1-one (39.6 g, 198.95 mmol, 9.96 equiv), and potassium carbonate (6.9 g, 49.92 mmol, 2.50 equiv) in 150 mL of acetonitrile was stirred at 80° C. overnight. The reaction mixture was cooled to room temperature and the solid was filtered out. The solution was diluted with ethyl acetate, washed with brine, dried over anhydrous sodium sulfate, and concentrated under vacuum. The residue was applied onto a silica gel column with ethyl acetate/petroleum ether (1:1). This resulted in 2.74 g (63%) of 1-methyl-3-(2-oxo-2-phenylethyl)imidazolidin-2-one as a brown solid.

The title compound was then prepared in an analogous manner to tert-butyl 4-((4-amino-1H-pyrazol-1-yl)(phenyl)methyl)-4-fluoropiperidine-1-carboxylate (Example A6), replacing tert-butyl 4-benzoyl-4-fluoro-piperidine-1-carboxylate (second step) with 1-methyl-3-(2-oxo-2-phenylethyl)imidazolidin-2-one.

### Example A73

### trans-1-(4-phenyltetrahydrofuran-3-yl)-1H-pyrazol-4-amine trans NH₂

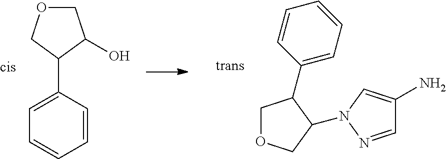 [SMILES: OC1COCC1c1ccccc1 ; Nc1cnn(C2COCC2c2ccccc2)c1]

Prepared in an analogous manner to 1-(3-(dimethylamino)-1-phenylpropyl)-1H-pyrazol-4-amine (Example A3), replacing 3-(dimethylamino)-1-phenyl-propan-1-ol with cis-4-phenyltetrahydrofuran-3-ol (see J. Am. Chem. Soc., 2004, 126, 13600).

### Example A74

### cis-1-(4-phenyltetrahydrofuran-3-yl)-1H-pyrazol-4-amine

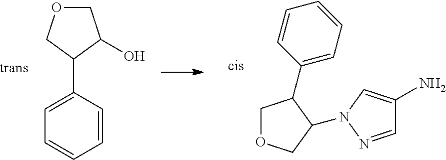 [SMILES: OC1COCC1c1ccccc1 ; Nc1cnn(C2COCC2c2ccccc2)c1]

Prepared in an analogous manner to 1-(3-(dimethylamino)-1-phenylpropyl)-1H-pyrazol-4-amine (Example A3), replacing 3-(dimethylamino)-1-phenyl-propan-1-ol with cis-4-phenyltetrahydrofuran-3-ol (see WO2007/90840 A1).

### Example A75

### 4-((4-amino-1H-pyrazol-1-yl)(tetrahydro-2H-pyran-4-yl)methyl)pyridin-2-amine

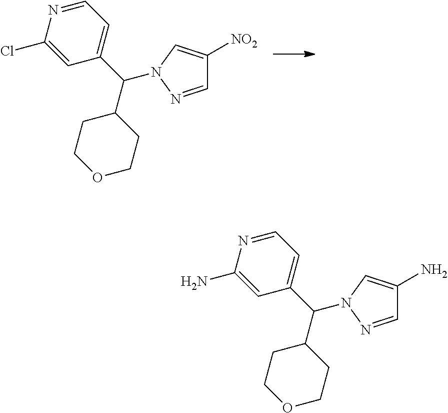 [SMILES: O=[N+]([O-])c1cnn(C(c2ccnc(Cl)c2)C2CCOCC2)c1 ; Nc1cnn(C(c2ccnc(N)c2)C2CCOCC2)c1]

The starting material 2-chloro-4-((4-nitro-1H-pyrazol-1-yl)(tetrahydro-2H-pyran-4-yl)methyl)pyridine was prepared in an analogous manner to 1-((3-(methylthio)phenyl)(tetrahydro-2H-pyran-4-yl)methyl)-4-nitro-1H-pyrazole (Example A69), replacing (3-(methylthio)phenyl)magnesium bromide with (2-chloropyridin-4-yl)lithium (formed in situ by treating a solution of 4-bromo-2-chloropyridine in THF with nBuLi at −78° C.).

A mixture of 2-chloro-4-((4-nitro-1H-pyrazol-1-yl)(tetrahydro-2H-pyran-4-yl)methyl)pyridine (1.61 g, 4.99 mmol, 1.00 equiv), NH₃.H₂O (5 mL, 28% in water), CuI (950 mg, 4.99 mmol, 1.00 equiv), ethane-1,2-diol (5 mL) was stirred for 12 h at 120° C. under nitrogen. The reaction was then quenched by the addition of water, extracted with ethyl acetate, dried over anhydrous sodium sulfate, and concentrated under vacuum. This resulted in 200 mg (13%) of tert-butyl 3-((4-nitro-1H-pyrazol-1-yl)(phenyl)methyl)thiomorpholine-4-carboxylate as a white solid.

### Example A76

### 2-((4-amino-1H-pyrazol-1-yl)(phenyl)methyl)tetrahydro-2H-thiopyran 1,1-dioxide

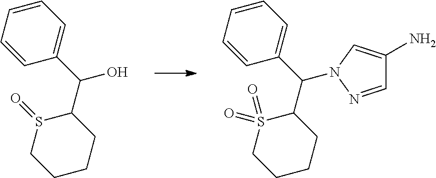 [SMILES: O=S1CCCCC1C(O)c1ccccc1 ; Nc1cnn(C(c2ccccc2)C2CCCCS2(=O)=O)c1]

Prepared in an analogous manner to 1-(3-(methylsulfonyl)-1-phenylpropyl)-1H-pyrazol-4-amine (Example A53), replacing 3-(methylsulfanyl)-1-phenylpropan-1-ol with 2-(hydroxy(phenyl)methyl)tetrahydro-2H-thiopyran 1-oxide (J. Am. Chem. Soc. 1999, 76, 617), and performing the nitro reduction step (final transformation) with palladium on carbon conditions as outlined in Example A3.

### Example A77

### 3-((4-amino-1H-pyrazol-1-yl)(phenyl)methyl)tetrahydro-2H-thiopyran 1,1-dioxide

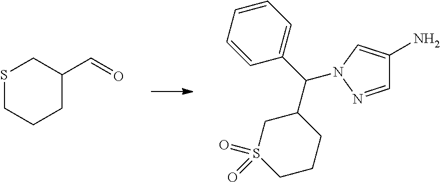 [SMILES: O=CC1CCCSC1 ; Nc1cnn(C(c2ccccc2)C2CCCS(=O)(=O)C2)c1]

Prepared in an analogous manner to 4-((4-amino-1H-pyrazol-1-yl)(phenyl)methyl)tetrahydro-2H-thiopyran 1,1-dioxide (Example A55), replacing tetrahydro-2H-thiopyran-4-carbaldehyde with tetrahydro-2H-thiopyran-3-carbaldehyde (WO2008/118724 A1).

### Example A78

### 3-((4-amino-1H-pyrazol-1-yl)(phenyl)methyl)tetrahydrothiophene 1,1-dioxide

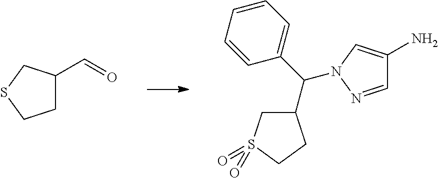 [SMILES: O=CC1CCSC1 ; Nc1cnn(C(c2ccccc2)C2CCS(=O)(=O)C2)c1]

Prepared in an analogous manner to 4-((4-amino-1H-pyrazol-1-yl)(phenyl)methyl)tetrahydro-2H-thiopyran 1,1-dioxide (Example A55), replacing tetrahydro-2H-thiopyran-4-carbaldehyde with tetrahydrothiophene-3-carbaldehyde (WO2008/118724 A1).

### Example A79a and A79b

### 4-((4-amino-1H-pyrazol-1-yl)(phenyl)methyl)-2-methyltetrahydro-2H-thiopyran 1,1-dioxide

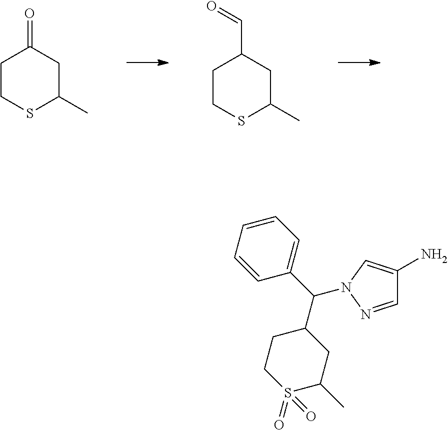

Under nitrogen n-BuLi (93 mL, 2.5 mol/L in hexanes) was added dropwise into a solution of (methoxymethyl)triphenylphosphanium chloride (66 g, 192.53 mmol, 2.51 equiv) in tetrahydrofuran (500 mL) at −15° C. A solution of 2-methylthian-4-one (10 g, 76.80 mmol, 1.00 equiv) in tetrahydrofuran (100 mL) was added dropwise at −15° C. The resulting solution was stirred for 2 h at −15° C. and then quenched by 200 mL of saturated NH₄Cl solution. The resulting solution was extracted with diethyl ether, dried over anhydrous sodium sulfate, and concentrated under vacuum. This resulted in 10 g of 4-(methoxymethylidene)-2-methylthiane as yellow oil.

A solution of 4-(methoxymethylidene)-2-methylthiane (24 g, 151.65 mmol, 1.00 equiv) in water (200 mL), propan-2-one (50 mL), PTSA (47 g, 272.94 mmol, 1.80 equiv) was heated to 50° C. for 1 h. The resulting solution was diluted with 2 L of diethyl ether, washed with brine, dried over anhydrous sodium sulfate, and concentrated under vacuum. The crude product was purified by distillation under reduced pressure (25 mm Hg) and the fraction was collected at 110° C. This resulted in 6 g (27%) of 2-methyltetrahydro-2H-thiopyran-4-carbaldehyde as a brown oil.

Under nitrogen PhMgBr (83 mL, 1M in THF) was added dropwise into a solution of 2-methylthiane-4-carbaldehyde (6 g, 41.60 mmol, 1.00 equiv) in tetrahydrofuran (500 mL) at 0° C. The resulting solution was stirred for 3 h at room temperature and then quenched by saturated NH₄Cl. The resulting solution was extracted with ethyl acetate and the organic layers were combined. The organic was washed with brine, dried over anhydrous sodium sulfate, and concentrated under vacuum. The residue was applied onto a silica gel column eluting with ethyl acetate/petroleum ether (1:100). This resulted in 3 g (32%) of (2-methylthian-4-yl)(phenyl)methanol as a colorless oil.

DIAD (5.454 g, 26.97 mmol, 2.00 equiv) was added dropwise into a solution of (2-methylthian-4-yl)(phenyl)methanol (3 g, 13.49 mmol, 1.00 equiv), 4-nitro-1H-pyrazole (2.29 g, 20.25 mmol, 1.50 equiv), PPh₃ (7.074 g, 26.97 mmol, 2.00 equiv) in tetrahydrofuran (100 mL). The resulting solution was stirred at room temperature overnight and quenched by saturated NH₄Cl solution. The resulting solution was extracted with ethyl acetate, washed with brine, dried over anhydrous sodium sulfate, and concentrated under vacuum. The residue was applied onto a silica gel column eluting with ethyl acetate/petroleum ether (1:100). This resulted in 3.7 g (86%) of 1-[(2-methylthian-4-yl)(phenyl)methyl]-4-nitro-1H-pyrazole as a yellow syrup.

m-CPBA (5.02 g, 29.09 mmol, 2.50 equiv) in ethyl acetate (50 mL) was added dropwise into a solution of 1-[(2-methylthian-4-yl)(phenyl)methyl]-4-nitro-1H-pyrazole (3.7 g, 11.66 mmol, 1.00 equiv) in dichloromethane (100 mL) at 0° C. The resulting solution was stirred for 3 h at room temperature, diluted with dichloromethane, washed with saturated sodium carbonate and brine, dried over anhydrous sodium sulfate, and concentrated under vacuum. The residue was applied onto a silica gel column eluting with ethyl acetate/petroleum ether (1:5). This resulted in isolation of two fractions each containing two diastereomers (four stereoisomers) of 2-methyl-4-[(4-nitro-1H-pyrazol-1-yl)(phenyl)methyl]-thiane-1,1-dione as a white solid (Fraction 1=1.5 g=37%; Fraction 2=0.5 g=12%). These fractions were used separately in subsequent operations.

Into a 500-mL round-bottom flask, was placed a solution of 2-methyl-4-[(4-nitro-1H-pyrazol-1-yl)(phenyl)methyl]-1 lambda6-thiane-1,1-dione (1.5 g, 4.29 mmol, 1.00 equiv; Fraction 1 from above) in ethyl acetate (300 mL), palladium on carbon (500 mg, 10%). The resulting solution was stirred for 3 h at room temperature under 1 atm of hydrogen. The solid was filtered out and the solution was concentrated under vacuum. This resulted in 1.5 g of 1-(phenyl(1,1-dioxo-2-methylthiane-4-yl)methyl)-1H-pyrazol-4-amine (Example A79a) as an off-white solid. Fraction 2 was treated similarly to obtain 300 mg of 1-(phenyl(1,1-dioxo-2-methylthiane-4-yl)methyl)-1H-pyrazol-4-amine (Example A79b).

### Example A80

### 2-((4-amino-1H-pyrazol-1-yl)(pyridin-3-yl)methyl)tetrahydro-2H-thiopyran 1,1-dioxide

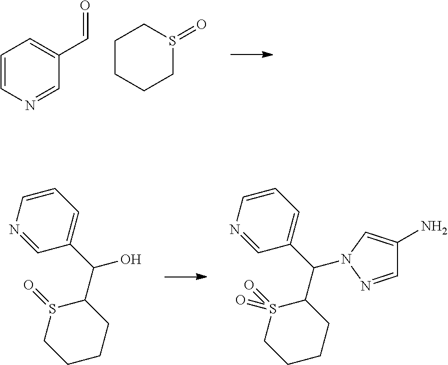

Under nitrogen n-BuLi (38.4 mL, 2.5 M in hexanes, 1.20 equiv) was added dropwise into a solution of thian-1-one (9.44 g, 79.87 mmol, 1.00 equiv) in tetrahydrofuran (150 mL) at −78° C. After 1.5 h pyridine-3-carbaldehyde (8.56 g, 79.92 mmol, 1.00 equiv) was added dropwise at −78° C. The resulting solution was stirred for 2 h at −78° C. and then was quenched by the addition of 50 mL of methanol. The resulting mixture was concentrated under vacuum and the residue was applied onto a silica gel column eluting with ethyl acetate/petroleum ether (1:5). This resulted in 11 g (61%) of 2-[hydroxy(pyridin-3-yl)methyl]-thian-1-one as a colorless oil.

The title compound was then prepared in an analogous manner to 2-((4-amino-1H-pyrazol-1-yl)(phenyl)methyl)tetrahydro-2H-thiopyran 1,1-dioxide (Example A76), replacing 2-(hydroxy(phenyl)methyl)tetrahydro-2H-thiopyran 1-oxide 2-[hydroxy(pyridin-3-yl)methyl]-thian-1-one.

### Example A81

### 2-((4-amino-1H-pyrazol-1-yl)(pyridin-3-yl)methyl)tetrahydro-2H-thiopyran 1,1-dioxide

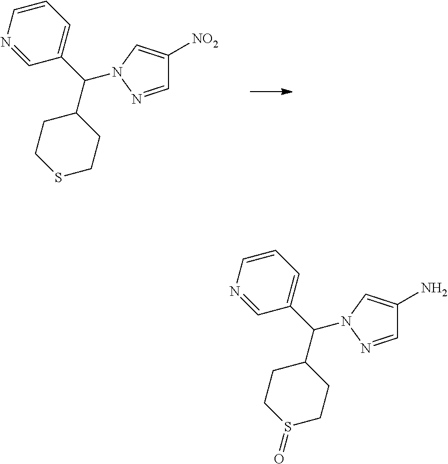 [SMILES: O=[N+]([O-])c1cnn(C(c2cccnc2)C2CCSCC2)c1 ; Nc1cnn(C(c2cccnc2)C2CCS(=O)CC2)c1]

Prepared in an analogous manner to 4-((4-amino-1H-pyrazol-1-yl)(phenyl)methyl)tetrahydro-2H-thiopyran 1-oxide (Example A66), replacing 4-nitro-1-[phenyl(thian-4-yl)methyl]-1H-pyrazole with 3-((4-nitro-1H-pyrazol-1-yl)(tetrahydro-2H-thiopyran-4-yl)methyl)pyridine (see Example A16), except in this case the mixture of diastereomers was carried forward.

### Example A82

### di-tert-butyl (2-((4-amino-1H-pyrazol-1-yl)(phenyl)methyl)propane-1,3-diyl)bis(methylcarbamate)

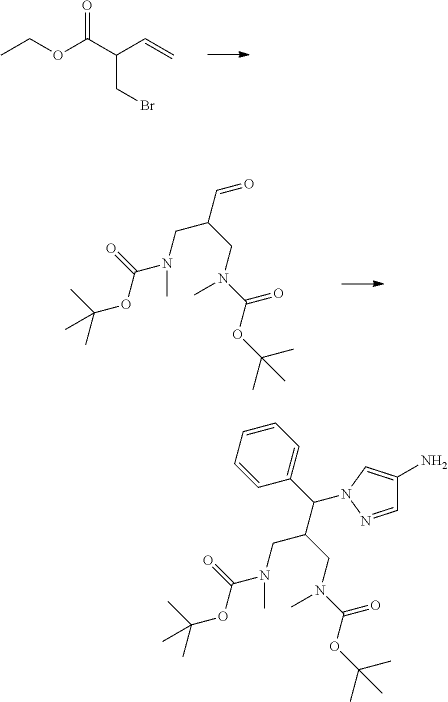

A solution of ethyl 2-(bromomethyl)prop-2-enoate (2.5 g, 12.95 mmol, 1.00 equiv) in CH₃CN (20 mL) and methylamine (33 mL, 2M in tetrahydrofuran) was stirred at room temperature overnight. The solid was filtered out and the solution was concentrated under vacuum. The residue was diluted with 100 mL of brine, extracted with diethyl ether and ethyl acetate, dried over anhydrous sodium sulfate, and concentrated under vacuum. This resulted in 1.8 g (80%) of ethyl 3-(methylamino)-2-[(methylamino)methyl]propanoate as colorless oil.

A solution of ethyl 3-(methylamino)-2-[(methylamino)methyl]propanoate (25.34 g, 145.43 mmol, 1.00 equiv), TEA (44.238 g, 437.18 mmol, 3.01 equiv), and Boc₂O (69.85 g, 320.05 mmol, 2.20 equiv) in dichloromethane (200 mL) was stirred at room temperature overnight. The reaction was diluted with 500 mL of ethyl acetate, washed with brine, dried over anhydrous sodium sulfate, and concentrated under vacuum. The residue was applied onto a silica gel column eluting with ethyl acetate/petroleum ether (1:100-1:10). This resulted in 17 g (31%) of ethyl 3-[[(tert-butoxy)carbonyl](methyl)amino]-2-([[(tert-butoxy)carbonyl](methyl)amino]methyl)propanoate as a yellow syrup.

Under nitrogen LiAlH₄ (5.1 g, 134.39 mmol, 5.03 equiv) was added in several batches to a stirred solution of ethyl 3-[[(tert-butoxy)carbonyl](methyl)amino]-2-([[(tert-butoxy)carbonyl](methyl)amino]methyl)propanoate (10 g, 26.70 mmol, 1.00 equiv) in tetrahydrofuran (130 mL) at 0° C. After 1 h the reaction was quenched by 5 mL of water/ice. NaOH solution (3N, 15 mL) was added and the precipitated solids were filtered out. The solution was extracted with ethyl acetate, washed with brine, dried over anhydrous sodium sulfate, and concentrated under vacuum. The residue was applied onto a silica gel column eluting with dichloromethane/methanol (50:1). This resulted in 1 g (11%) of tert-butyl N-[2-([[(tert-butoxy)carbonyl](methyl)amino]methyl)-3-hydroxypropyl]-N-methylcarbamate as a colorless oil.

A solution of tert-butyl N-[2-([[(tert-butoxy)carbonyl](methyl)amino]methyl)-3-hydroxypropyl]-N-methylcarbamate (1.82 g, 5.47 mmol, 1.00 equiv) and DMP (2.76 g, 6.51 mmol, 1.19 equiv) in dichloromethane (150 mL) was stirred at room temperature overnight. The resulting solution was diluted with ethyl acetate, washed with saturated sodium carbonate and brine, dried over anhydrous sodium sulfate, and concentrated under vacuum. The residue was applied onto a silica gel column eluting with ethyl acetate/petroleum ether (1:5). This resulted in 700 mg (39%) of tert-butyl N-[2-([[(tert-butoxy)carbonyl](methyl)amino]methyl)-3-oxopropyl]-N-methylcarbamate as a light yellow syrup.

The title compound was then prepared in an analogous manner to 1-((3-chlorophenyl)(tetrahydro-2H-pyran-4-yl)methyl)-1H-pyrazol-4-amine (Example A48), replacing 3-chlorophenylmagnesium bromide with phenylmagnesium bromide and tetrahydropyran-4-carbaldehyde with tert-butyl N-[2-([[(tert-butoxy)carbonyl](methyl)amino]methyl)-3-oxopropyl]-N-methylcarbamate.

### Example A83

### 1-(phenyl(tetrahydro-2H-thiopyran-4-yl)methyl)-1H-pyrazol-4-amine

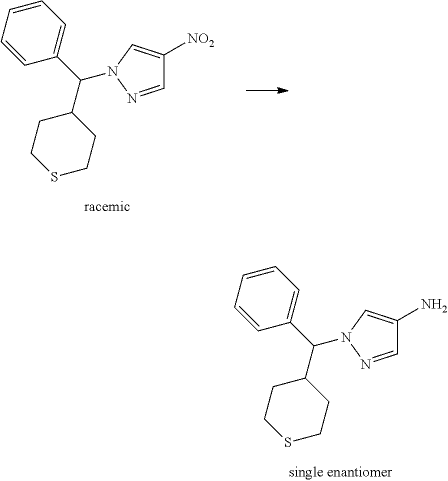 [SMILES: O=[N+]([O-])c1cnn(C(c2ccccc2)C2CCSCC2)c1 ; Nc1cnn(C(c2ccccc2)C2CCSCC2)c1]

4-Nitro-1-(phenyl(tetrahydro-2H-thiopyran-4-yl)methyl)-1H-pyrazole (intermediate en route to Example A55) was separated into its constituent enantiomers using SFC with a chiral stationary phase. A small amount of each enantiomer was carried forward to Examples 60a and 60b using procedures outlined below. The enantiomer of 4-nitro-1-(phenyl(tetrahydro-2H-thiopyran-4-yl)methyl)-1H-pyrazole which provided Example 60a was then reduced to the aminopyrazole using procedures outlined above, and used as Example A83.

### Synthesis of Ketones

### Examples B

Many ketones are commercially available (or known in the literature) and are used directly in the syntheses of pyrazole carboxylates (Examples C). Syntheses for previously unknown ketones are outlined below.

### Example B1

### 4-(1-((2-(trimethylsilyl)ethoxy)methyl)-1H-pyrazol-4-yl)cyclohexanone

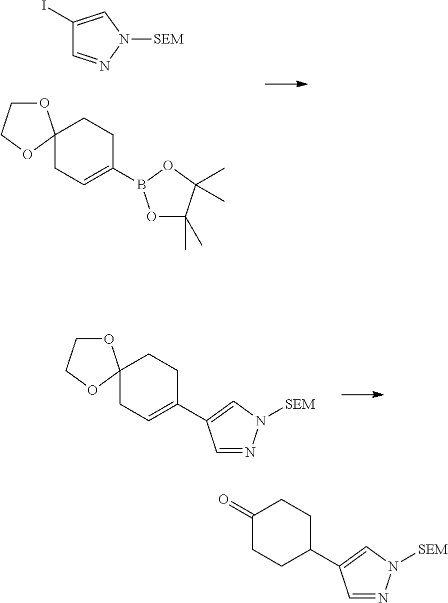 [SMILES: C[Si](C)(C)CCOCn1cc(I)cn1 ; CC1(C)OB(C2=CCC3(CC2)OCCO3)OC1(C)C ; C[Si](C)(C)CCOCn1cc(C2=CCC3(CC2)OCCO3)cn1 ; C[Si](C)(C)CCOCn1cc(C2CCC(=O)CC2)cn1]

To a solution of 2-[(4-iodopyrazol-1-yl)methoxy]ethyl-trimethyl-silane (1.000 g, 3.08 mmol; see Bioorg. Med. Chem. Lett. 2004, 14, 3063) in acetonitrile (23 mL) was added 2-(1,4-dioxaspiro[4.5]dec-8-en-8-yl)-4,4,5,5-tetramethyl-1,3,2-dioxaborolane; 1.30 equiv.; 4.009 mmol), sodium carbonate (493 mg, 1.50 equiv., 4.62 mmol), tetrakistriphenylphosphinepalladium(0) (184 mg, 0.050 equiv., 0.1542 mmol) and deoxygenated water (13 mL). The mixture was heated to 90° C. and stirred for 3 days. The mixture was diluted with water, extracted 3× with EtOAc, then the combined organic extracts were dried (MgSO₄) and concentrated in vacuo. Purification by CombiFlash (40 g; 100:0 to 70:30 heptane:EtOAc) provided 2-[[4-(1,4-dioxaspiro[4.5]dec-8-en-8-yl)pyrazol-1-yl]methoxy]ethyl-trimethyl-silane (881 mg, 2.62 mmol, 85%). This material was diluted with methanol (20 mL), then 10% palladium on carbon (228 mg) was added and the mixture was stirred under an atmosphere of hydrogen at 65° C. overnight. After cooling to rt, the mixture was filtered through Celite, concentrated in vacuo and used directly. This material was diluted with glacial acetic acid (10 mL) and water (3 mL) and heated to 65° C. overnight. The mixture was diluted with sat. NaHCO₃(aq) and washed with 10% MeOH in CH₂Cl₂ (3×). The combined organic extracts were dried (MgSO₄) and concentrated in vacuo. Purification by CombiFlash (40 g; 100:0 to 0:100 heptane:EtOAc) provided the title compound (620 mg, 2.10 mmol, 68%).

### Example B2

### 4-(pyrimidin-5-yl)cyclohexanone

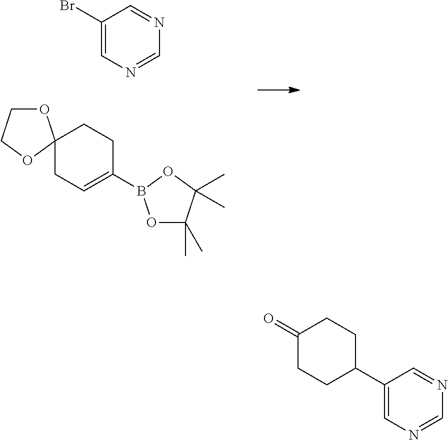 [SMILES: Brc1cncnc1 ; CC1(C)OB(C2=CCC3(CC2)OCCO3)OC1(C)C ; O=C1CCC(c2cncnc2)CC1]

Prepared in an analogous manner to 4-(1-((2-(trimethylsilyl)ethoxy)methyl)-1H-pyrazol-4-yl)cyclohexanone (Example B1), replacing 2-[(4-iodopyrazol-1-yl)methoxy]ethyl-trimethyl-silane with 5-bromopyrimidine.

### Example B3

### 2-oxaspiro[3.5]nonan-7-one

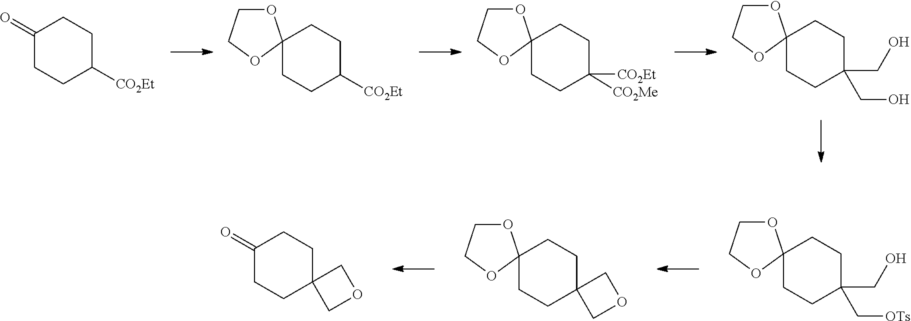

Step 1:

To a solution of ethyl 4-oxocyclohexanecarboxylate (35 g, 0.21 mol, 1.0 equiv.) in toluene, ethylene glycol (26 g, 0.42 mol, 2 equiv.) and TsOH (500 mg) were added. The mixture was stirred at room temperature under N₂ overnight. The mixture was concentrated and then extracted with EtOAc, washed by water, brine and dried over anhydrous Na₂SO₄. Concentration in vacuum provided ethyl 1,4-dioxaspiro[4.5]decane-8-carboxylate. (30 g, colorless oil, yield: 68%).

Step 2:

To a solution of ethyl 1,4-dioxaspiro[4.5]decane-8-carboxylate (120 g, 0.56 mol, 1.0 equiv.) in THF was added LDA (336 ml, 2 M, 0.67 mol, 1.2 equiv.) dropwise at −78° C. under N₂. Then it was stirred at −78° C. for 1 h. Dimethyl carbonate (55.5 g, 0.62 mol, 1.1 equiv.) was added dropwise at −78° C. The mixture was stirred at room temperature for another 1 h. NH₄C(aq.) was added. It was extracted with EtOAc, washed by water, brine and dried over anhydrous Na₂SO₄. Concentration in vacuum and chromatography on silica gel (eluting conditions) provided 8-ethyl 8-methyl 1,4-dioxaspiro[4.5]decane-8,8-dicarboxylate. (110 g, colorless oil, yield: 70%)

Step 3:

To a solution of 8-ethyl 8-methyl 1,4-dioxaspiro[4.5]decane-8,8-dicarboxylate (54.4 g, 0.2 mol, 1.0 equiv.) in THF was added LAH (22.8 g, 0.6 mol, 3.0 equiv.) maintaining temperature below 50° C. Then it was stirred at 70° C. for another 1 h. The reaction was quenched by the addition of Na₂SO₄.10H₂O. After filtration, the mixture was diluted with EtOAc, washed by water, brine and dried over anhydrous Na₂SO₄. Concentration gave 1,4-dioxaspiro[4.5]decane-8,8-diyldimethanol. (16 g, white solid, yield: 40%)

Step 4:

To a solution of 1,4-dioxaspiro[4.5]decane-8,8-diyldimethanol (15 g, 0.074 mol, 1.0 equiv.) in THF (200 mL) was added NaH (4.46 g, 60%, 0.11 mol, 1.5 equiv.) at 0° C. Then it was stirred at room temperature for 1 h. TsCl (14.16 g, 0.074 mol, 1.0 equiv.) in THF was added dropwise at 0° C. under N₂ and stirred for another 1 h. The mixture was extracted with EtOAc, washed by water, brine and dried over anhydrous Na₂SO₄. Concentration and chromatography on silica gel gave (8-(hydroxymethyl)-1,4-dioxaspiro[4.5]decan-8-yl)methyl 4-methylbenzenesulfonate. (16 g, colorless oil, yield: 60%)

Step 5:

To a solution of (8-(hydroxymethyl)-1,4-dioxaspiro[4.5]decan-8-yl)methyl 4-methylbenzenesulfonate (15 g, 0.042 mol, 1.0 equiv.) in THF (200 mL) was added NaH (3.4 g, 60%, 0.084 mol, 2.0 equiv.) at bellow 20° C. The mixture was stirred at 70° C. for 5 h. It was cooled to room temperature and extracted with EtOAC, washed by water, brine and dried over anhydrous Na₂SO₄. Concentration and chromatography on silica gel provided ketal-protected 2-oxaspiro[3.5]nonan-7-one. (6 g, white solid, yield: 77%)

Step 6:

To a solution of ketal-protected 2-oxaspiro[3.5]nonan-7-one (5 g, 0.027 mol, 1.0 equiv.) in acetone (100 mL) was added pyridinium tosylate (2.0 g, 0.08 mol, 0.3 equiv.). Then it was stirred at 60° C. overnight. The mixture was concentrated and then extracted with EtOAc, washed by water, brine and dried over anhydrous Na₂SO₄. Concentration and chromatography on silica gel gave 2-oxaspiro[3.5]nonan-7-one (1.5 g, light yellow solid, yield=30%) and 2.0 g recovered ketal starting material which can be recycled to provide additional 2-oxaspiro[3.5]nonan-7-one.

### Example B4

### 4-((tert-butyldimethylsilyloxy)methyl)-4-methylcyclohexanone

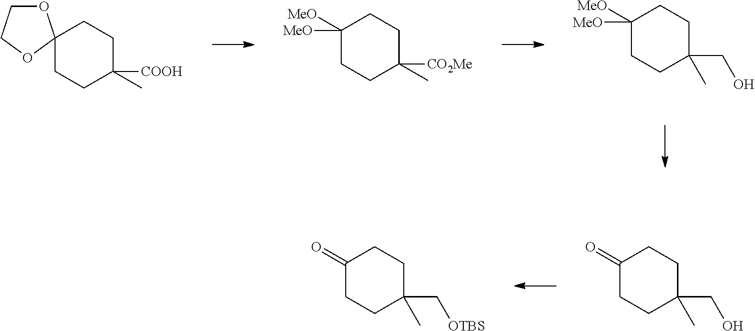

Step 1:

To a solution of 8-methyl-1,4-dioxaspiro[4.5]decane-8-carboxylic acid (1.00 g, 4.99 mmol) in 8 mL of MeOH was added conc. HCl(aq) (0.18 mL) and the mixture was heated to 65° C. for 3 days. The mixture was poured into sat. NaHCO₃(aq) and extracted 3 times with EtOAc. The combined organic extracts were dried (MgSO₄) and concentrated in vacuo. Purification by CombiFlash (40 g; 100:0 to 70:30 heptane:EtOAc over 28 minutes) provided 423 mg (1.96 mmol) of methyl 4,4-dimethoxy-1-methylcyclohexanecarboxylate. A small amount (180 mg) of the glycol ketal is also obtained, and can be used in subsequent reactions in an identical manner to the dimethyl ketal.

Step 2:

A solution of methyl 4,4-dimethoxy-1-methylcyclohexanecarboxylate (423 mg, 1.96 mmol) in CH₂Cl₂ (11 mL) was cooled to −78° C., then Dibal (1.0 M in CH₂Cl₂, 3.90 mL, 3.90 mmol) was added dropwise. The mixture was allowed to warm to room temperature overnight. The mixture was re-cooled to 0° C., then quenched by the addition of MeOH, then H₂O. After stirring for 30 minutes, the mixture was filtered through Celite, dried (MgSO₄) and concentrated in vacuo. Purification by CombiFlash (12 g; 100:0 to 0:100 heptane:EtOAc over 24 minutes) provided 269 mg (1.43 mmol) of (4,4-dimethoxy-1-methylcyclohexyl)methanol.

Step 3:

A solution of (4,4-dimethoxy-1-methylcyclohexyl)methanol (269 mg, 1.43 mmol) in glacial acetic acid (5 mL) and water (1.5 mL) was heated to 65° C. overnight. After cooling to room temperature the mixture was neutralized to pH 8 with sat. NaHCO₃(aq) and extracted with 10% MeOH/CH₂Cl₂ until no product was detectable in the aqueous layer by TLC (8 times). The combined extracts were dried (MgSO₄) and concentrated in vacuo. Purification by CombiFlash (12 g; 100:0 to 0:100 heptane:EtOAc) provided 121 mg (0.853 mmol) of 4-(hydroxymethyl)-4-methylcyclohexanone.

Step 4:

To a solution of 4-(hydroxymethyl)-4-methylcyclohexanone (121 mg, 0.853 mmol) in THF (2 mL) was added imidazole (117 mg, 1.71 mmol), TBSCl (140 mg, 0.904 mmol) and DMF (3 μL). The mixture was heated to 65° C., then cooled to rt. The mixture was diluted with sat. NH₄Cl(aq) and extracted with EtOAc (3 times). The combined organic extracts were dried (MgSO₄) and concentrated in vacuo. Purification by CombiFlash (12 g; 100:0 to 80:20 heptane:EtOAc) provided 119 mg (0.467 mmol) of 4-((tert-butyldimethylsilyloxy)methyl)-4-methylcyclohexanone.

### Example B5

### 4-((tert-butyldimethylsilyl)oxy)-4-methylcyclohexanone

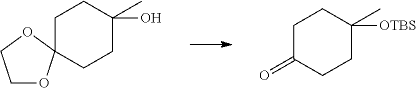 [SMILES: CC1(O)CCC2(CC1)OCCO2 ; CC1(O[Si](C)(C)C(C)(C)C)CCC(=O)CC1]

8-Methyl-1,4-dioxaspiro[4.5]decan-8-ol (1.61 g, 9.35 mmol, see WO2011/139107 A2) in dry tetrahydrofuran (24 mL) was added tert-butyldimethylsilyl chloride (1.06 equiv., 9.91 mmol, 1.54 g) followed by imidazole (2.01 equiv., 18.8 mmol, 1.29 g) and followed by N,N-dimethylformamide (0.05 equiv., 0.467 mmol, 0.036 mL) The sample was heated at 86° C. for 3 days. The sample was diluted with water, then extracted 3 times with dichloromethane, dried over MgSO₄, filtered and evaporated. Purification by CombiFlash (40 g, 0-50% EtOAc in heptane, 14 min gradient) provided tert-butyl-dimethyl-[(8-methyl-1,4-dioxaspiro[4.5]decan-8-yl)oxy]silane (1.03 g, 3.61 mmol, 39% yield).

Tert-butyl-dimethyl-[(8-methyl-1,4-dioxaspiro[4.5]decan-8-yl)oxy]silane (0.99 g, 3.46 mmol), glacial acetic acid (13 mL), and water (3.2 mL) were combined and heated to 65° C. for 2 hours. The sample was concentrated, diluted with sat NaHCO₃ and extracted 3 times with 10% MeOH in dichloromethane, dried over MgSO₄, filtered and concentrated in vacuo. Purification by CombiFlash (12 g, 0-20% EtOAc in heptane, 11 min gradient) provided 4-[tert-butyl(dimethyl)silyl]oxy-4-methyl-cyclohexanone (763 mg, 3.14 mmol, 91% yield).

### Example B6

### 4-ethyl-4-methylcyclohexanone

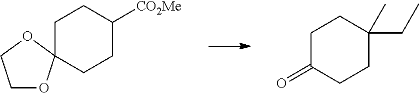

To diisopropylamine (1.70 equiv., 5.937 mmol, 0.836 mL) in dry tetrahydrofuran (61 mL) at −78° C. was added butyllithium (1.6 mol/L) in hexanes (1.50 equiv., 5.24 mmol, 3.30 mL) dropwise. The sample was stirred at −78° C. for 5 min. The LDA solution was then added dropwise by cannula to a solution of methyl 1,4-dioxaspiro[4.5]decane-8-carboxylate (0.699 g, 3.49 mmol) in dry tetrahydrofuran (16 mL) at −78° C. Iodoethane (1.50 equiv., 5.24 mmol, 0.423 mL) was then added dropwise immediately to the reaction mixture. The sample was allowed to warm slowly to room temperature and stirred overnight. Sat NH₄Cl (10 mL) was added dropwise to the sample, which was then diluted with H₂O and extracted 3 times with EtOAc, dried over MgSO₄, filtered, and evaporated. Purification by CombiFlash (12 g, 0-20% EtOAc in heptane, 11 min gradient) provided methyl 8-ethyl-1,4-dioxaspiro[4.5]decane-8-carboxylate (626 mg, 2.74 mmol, 79% yield).

To a solution of methyl 8-ethyl-1,4-dioxaspiro[4.5]decane-8-carboxylate (0.626 g, 2.745 mmol) in dry tetrahydrofuran (6 mL) at 0° C. was added 2.0 M lithium aluminium hydride in tetrahydrofuran (2.43 equiv., 6.67 mmol, 3.34 mL) dropwise. The sample was allowed to warm slowly to room temperature and stirred for 3 days. The mixture was cooled to 0° C., followed by sequential dropwise addition of water (0.25 mL), 15% NaOH(aq) (0.25 mL) then water (0.75 mL). The sample was warmed to room temperature and stirred for 1 hour. The sample was vacuum filtered through a pad of celite, evaporated, and purified by CombiFlash (12 g, 0-100% EtOAc in heptane, 11 min gradient) to provide (8-ethyl-1,4-dioxaspiro[4.5]decan-8-yl)methanol (311 mg, 1.55 mmol, 57% yield).

To a solution of (8-ethyl-1,4-dioxaspiro[4.5]decan-8-yl)methanol (0.311 g, 1.55 mmol) in dry dichloromethane (1.55 mL) and dry pyridine (2.50 equiv., 3.89 mmol, 0.32 mL) at 0° C. was added p-toluenesulfonyl chloride (1.20 equiv., 1.866 mmol, 363 mg). The sample was allowed to warm slowly to room temperature and stirred overnight. Since starting material was still evident by TLC, additional p-toluenesulfonyl chloride (363 mg), pyridine (0.32 mL), and 4-(dimethylamino)pyridine (0.05 equiv., 0.077 mmol, 10 mg) were added and the sample was stirred for 1 hour. The sample was diluted with water, extracted 3 times with dichloromethane, dried over MgSO₄, filtered, evaporated, and purified by CombiFlash (12 g, 0-40% EtOAc in heptane, 11 min gradient) to provide (8-ethyl-1,4-dioxaspiro[4.5]decan-8-yl)methyl 4-methylbenzenesulfonate (0.509 mg, 1.43 mmol, 92% yield).

To a solution of (8-ethyl-1,4-dioxaspiro[4.5]decan-8-yl)methyl 4-methylbenzenesulfonate (0.4391 g, 1.239 mmol) in dry tetrahydrofuran (2.6 mL) at 0° C. was added 2.0 M lithium aluminium hydride in tetrahydrofuran (2.43 equiv., 3.010 mmol, 1.50 mL) dropwise. The sample was then heated to 66° C. and stirred overnight. The mixture was cooled to 0° C., followed by the sequential addition of water (0.11 mL), 15% NaOH(aq) (0.11 mL) and water (0.33 mL). The sample was warmed to room temperature and stirred for 1 hour. The sample was vacuum filtered through a pad of celite, evaporated, and purified by CombiFlash (12 g, 0-20% EtOAc in heptane, 11 min gradient) to provide 8-ethyl-8-methyl-1,4-dioxaspiro[4.5]decane (200 mg, 1.09 mmol, 88% yield).

8-Ethyl-8-methyl-1,4-dioxaspiro[4.5]decane (0.200 g, 1.087 mmol), glacial acetic acid (4 mL) and water (1 mL) were combined and heated to 65° C. for 3 hours. The sample was concentrated, diluted with sat NaHCO₃, extracted 3 times with 10% MeOH in dichloromethane, dried over MgSO₄, filtered, and evaporated to provide 4-ethyl-4-methyl-cyclohexanone (152 mg, 1.09 mmol, 100% yield).

### Example B7

### 2-oxaspiro[4.5]decan-8-one

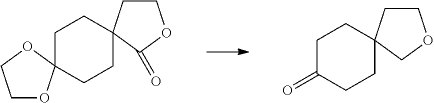 [SMILES: O=C1OCCC12CCC1(CC2)OCCO1 ; O=C1CCC2(CCOC2)CC1]

To a solution of 2-oxaspiro[4.5]decane-1,8-dione ethylene ketal (0.730 g, 3.44 mmol, see U.S. Pat. No. 4,588,591 A1) in dry tetrahydrofuran (7.3 mL) at 0° C. was added lithium aluminum hydride (2.0 mol/L) in THF (1.70 equiv., 5.85 mmol, 2.90 mL) dropwise. The sample was allowed to warm slowly to room temperature and stirred overnight. The sample was cooled to 0° C. followed by sequential addition of water (0.22 mL), 15% NaOH(aq) (0.22 mL) and water (0.66 mL). The sample was warmed to room temperature and stirred for 1 hour. The sample was vacuum filtered through a pad of celite and concentrated in vacuo to provide 2-[8-(hydroxymethyl)-1,4-dioxaspiro[4.5]decan-8-yl]ethanol (744 mg; 3.44 mmol, 100% yield).

To a solution of 2-[8-(hydroxymethyl)-1,4-dioxaspiro[4.5]decan-8-yl]ethanol (0.840 g, 3.88 mmol) in dry tetrahydrofuran (7 mL) at 0° C. was added triphenylphosphine (2.00 equiv., 7.77 mmol, 2.08 g) followed by diethyl azodicarboxylate (2.00 equiv., 7.77 mmol, 1.61 mL) dropwise. The sample was allowed to warm slowly to room temperature and stirred overnight. The sample was diluted with H₂O, extracted 3 times with CH₂Cl₂, dried over MgSO₄, filtered and evaporated. Purification by CombiFlash (80 g, 0-30% EtOAc in heptane, 25 min gradient) provided 2-oxaspiro[4.5]decan-8-one ethylene ketal (600 mg, 3.00 mmol, 78% yield).

2-Oxaspiro[4.5]decan-8-one ethylene ketal (0.75 g, 3.8 mmol), glacial acetic acid (14 mL), and water (3.5 mL) were combined and heated to 65° C. for 3 days. The sample was concentrated, diluted with sat NaHCO₃, extracted 3 times with 10% MeOH in dichloromethane, dried over MgSO₄, filtered and evaporated. Purification by CombiFlash (40 g, 0-100% EtOAc in heptane, 14 min gradient) provided 3-oxaspiro[4.5]decan-8-one (499 mg, 3.23 mmol, 85% yield).

### Example B8

### 2-methyl-2-azaspiro[4.5]decane-1,8-dione

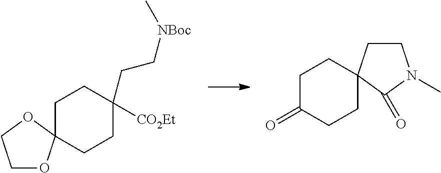 [SMILES: CCOC(=O)C1(CCN(C)C(=O)OC(C)(C)C)CCC2(CC1)OCCO2 ; CN1CCC2(CCC(=O)CC2)C1=O]

Ethyl 8-(2-((tert-butoxycarbonyl)(methyl)amino)ethyl)-1,4-dioxaspiro[4.5]decane-8-carboxylate was obtained in an analogous manner to methyl 8-ethyl-1,4-dioxaspiro[4.5]decane-8-carboxylate (see Example B6), replacing ethyl iodide with tert-butyl N-(2-iodoethyl)-N-methyl-carbamate (see US2007/4675 A1), and replacing methyl 1,4-dioxaspiro[4.5]decane-8-carboxylate with ethyl 1,4-dioxaspiro[4.5]decane-8-carboxylate.

Ethyl 8-[2-[tert-butoxycarbonyl(methyl)amino]ethyl]-1,4-dioxaspiro[4.5]decane-8-carboxylate (1.85 g, 4.98 mmol) and trifluoroacetic acid (19 mL) were combined and stirred overnight. The sample was evaporated. 1,2-dichloroethane (28 mL) followed by N,N′-diisopropylethylamine (43 mL) was added to the sample. The sample was heated to 83° C. for 1.5 hours. The sample was evaporated, and purified by CombiFlash (80 g, 0-100% EtOAc in heptane, 25 min gradient, 25 min isocratic at 100% EtOAc) to provide 3-methyl-3-azaspiro[4.5]decane-4,8-dione (0.77 g, 4.2 mmol, 85% yield).

### Example B9

### 2-oxaspiro[4.4]nonan-7-one

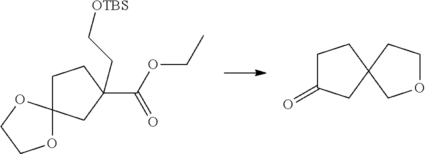 [SMILES: CCOC(=O)C1(CCO[Si](C)(C)C(C)(C)C)CCC2(C1)OCCO2 ; O=C1CCC2(CCOC2)C1]

Ethyl 7-(2-((tert-butyldimethylsilyl)oxy)ethyl)-1,4-dioxaspiro[4.4]nonane-7-carboxylate was obtained in an analogous manner to methyl 8-ethyl-1,4-dioxaspiro[4.5]decane-8-carboxylate (see Example B6), replacing ethyl iodide with (2-bromoethoxy)-tert-butyldimethylsilane, and replacing methyl 1,4-dioxaspiro[4.5]decane-8-carboxylate with ethyl 6,9-dioxaspiro[4.4]nonane-3-carboxylate.

Ethyl 3-[2-[tert-butyl(dimethyl)silyl]oxyethyl]-6,9-dioxaspiro[4.4]nonane-3-carboxylate (6.28 g, 17.5 mmol) and 1.0 M tetra-n-butylammonium fluoride in THF (2.00 equiv., 35.0 mmol, 35 mL) were combined and stirred for 30 min. The sample was diluted with H₂O, extracted 3 times with EtOAc, dried over MgSO₄, filtered and concentrated in vacuo. Purification by CombiFlash (80 g, 0-100% EtOAc in heptane, 25 min gradient) provided 2-oxaspiro[4.4]nonane-1,7-dione ethylene ketal (2.68 g, 13.5 mmol, 77% yield).

2-oxaspiro[4.4]nonan-7-one was then obtained in a manner analogous to 2-oxaspiro[4.5]decan-8-one (Example B7), replacing 2-oxaspiro[4.5]decane-1,8-dione ethylene ketal with 2-oxaspiro[4.4]nonane-1,7-dione ethylene ketal.

### Example B10

### 2-oxaspiro[3.4]octan-6-one

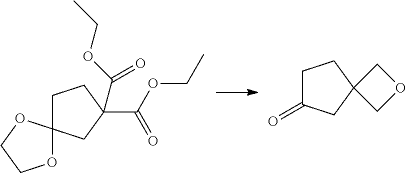 [SMILES: CCOC(=O)C1(C(=O)OCC)CCC2(C1)OCCO2 ; O=C1CCC2(COC2)C1]

Diethyl 1,4-dioxaspiro[4.4]nonane-7,7-dicarboxylate was obtained in an analogous manner to methyl 8-ethyl-1,4-dioxaspiro[4.5]decane-8-carboxylate (see Example B6), replacing ethyl iodide with ethyl chloroformate, and replacing methyl 1,4-dioxaspiro[4.5]decane-8-carboxylate with ethyl 6,9-dioxaspiro[4.4]nonane-3-carboxylate.

To a solution of diethyl 1,4-dioxaspiro[4.4]nonane-7,7-dicarboxylate (0.6558 g, 2.408 mmol) in dry THF (5 mL) at 0° C. was added lithium aluminum hydride (2.0 mol/L) in THF (3.40 equiv., 8.188 mmol, 4.1 mL) dropwise. The sample was allowed to warm slowly to room temperature and stirred overnight. The sample was cooled to 0° C. followed by sequential addition of water (0.31 mL), 15% NaOH(aq) (0.31 mL) and water (0.93 mL). The sample was warmed to room temperature and stirred for 1 hour. The sample was vacuum filtered through a pad of celite, evaporated, and vacuum pump dried for 3 hours to provide [3-(hydroxymethyl)-6,9-dioxaspiro[4.4]nonan-3-yl]methanol (411 mg, 2.18 mmol, 91% yield).

To a solution of [3-(hydroxymethyl)-6,9-dioxaspiro[4.4]nonan-3-yl]methanol (3.12 g, 16.6 mmol) in dry tetrahydrofuran (120 mL) at −78° C. was added n-butyllithium (1.6 mol/L) in hexanes (1.00 equiv., 16.6 mmol, 10.4 mL) dropwise. The sample was stirred at −78° C. for 30 min. p-Toluenesulfonyl chloride (1.00 equiv., 16.6 mmol, 3.22 g) in dry tetrahydrofuran (31 mL) was added dropwise to the sample. The sample was warmed to room temperature and stirred for 1 hour. 25% sodium methoxide in MeOH (2.00 equiv., 33.2 mmol, 7.6 mL) was then added dropwise to the sample. The sample was then heated to 66° C. and stirred overnight. Sat NH₄Cl(aq) (24 mL) was then added dropwise to the sample. The sample was extracted 3 times with EtOAc, dried over MgSO₄, filtered, and evaporated. Purification by CombiFlash (40 g, 0-100% EtOAc in heptane) provided 2-oxaspiro[3.4]octan-6-one ethylene ketal (1.85 g; 10.9 mmol, 66% yield).

The title compound was obtained by deprotection of the ethylene ketal in an analogous manner as described in the final step of 2-oxaspiro[4.5]decan-8-one (Example B7), replacing 2-oxaspiro[4.5]decan-8-one ethylene ketal with 2-oxaspiro[3.4]octan-6-one ethylene ketal, and reducing heating time to 24 hours.

### Example B11

### 1-((tert-butyldimethylsilyl)oxy)spiro[4.5]decan-8-one

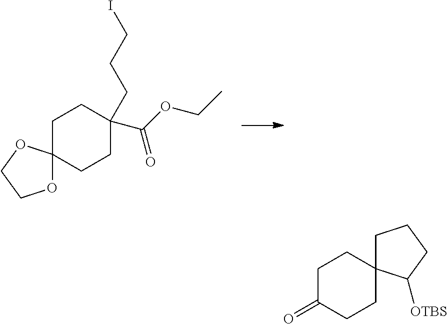 [SMILES: CCOC(=O)C1(CCCI)CCC2(CC1)OCCO2 ; CC(C)(C)[Si](C)(C)OC1CCCC12CCC(=O)CC2]

Ethyl 8-(3-iodopropyl)-1,4-dioxaspiro[4.5]decane-8-carboxylate was obtained in an analogous manner to methyl 8-ethyl-1,4-dioxaspiro[4.5]decane-8-carboxylate (see Example B6), replacing ethyl iodide with 1,3-diiodopropane, and replacing methyl 1,4-dioxaspiro[4.5]decane-8-carboxylate with ethyl 1,4-dioxaspiro[4.5]decane-8-carboxylate.

Ethyl 8-(3-iodopropyl)-1,4-dioxaspiro[4.5]decane-8-carboxylate (3.16 g, 8.27 mmol) in dry tetrahydrofuran (60 mL) at −78° C. was added samarium(II) iodide (0.1 mol/L) in THF (2.00 equiv., 16 mmol, 160 mL) dropwise. The sample was allowed to warm slowly to room temperature overnight, then was heated to 66° C. for 24 hours. The sample was diluted with brine, extracted 3 times with EtOAc, dried over MgSO₄, filtered, and concentrated in vacuo. Purification by CombiFlash (40 g, 0-50% EtOAc in heptane, 50 min gradient) provides spiro[4.5]decane-1,8-dione 8,8-ethylene ketal (351 mg, 1.67 mmol, 20% yield) and 1-hydroxyspiro[4.5]decan-8-one ethylene ketal (133 mg, 0.63 mmol, 8% yield).

A solution of spiro[4.5]decane-1,8-dione 8,8-ethylene ketal (0.402 g, 1.91 mmol) in dry ethanol (10 mL) was added sodium borohydride (2.00 equiv., 3.82 mmol, 148 mg) slowly. The sample was stirred for 1 hour. The sample was quenched by adding sat NaHCO₃, extracted 3 times with 10% MeOH in dichloromethane, dried over MgSO₄, filtered, and concentrated in vacuo. Purification by ComibFlash (12 g, 0-50% EtOAc in heptane, 11 min gradient) provided 1-hydroxyspiro[4.5]decan-8-one ethylene ketal (406 mg, 1.91 mmol, 100% yield).

A solution of 1-hydroxyspiro[4.5]decan-8-one ethylene ketal (0.6151 g, 2.90 mmol) in dry tetrahydrofuran (7.5 mL) was added tert-butyldimethylsilyl chloride (1.06 equiv., 3.07 mmol, 477 mg) followed by imidazole (2.01 equiv., 5.824 mmol, 400 mg) followed by N,N-dimethylformamide (0.05 equiv., 0.01 mL). The sample was heated to 66° C. and stirred overnight. Since TLC still shows starting material, additional tert-butyldimethylsilyl chloride (1.06 equiv., 3.07 mmol, 477 mg), imidazole (1.00 equiv., 2.90 mmol, 199 mg) and dry N,N-dimethylformamide (0.05 equiv., 0.01 mL) were added to the sample, and heating was continued for an additional 24 hours. The sample was diluted with sat NH₄Cl, extracted 3 times with EtOAc, dried over MgSO₄, filtered, and concentrated in vacuo. Purification by CombiFlash (40 g, 0-20% EtOAc in heptane, 28 min gradient) provided 1-((tert-butyldimethylsilyl)oxy)spiro[4.5]decan-8-one ethylene ketal (775 mg, 2.37 mmol, 82% yield).

The title compound was obtained by deprotection of the ethylene ketal in an analogous manner as described in the final step of 2-oxaspiro[4.5]decan-8-one (Example B7), replacing 2-oxaspiro[4.5]decan-8-one ethylene ketal with 1-((tert-butyldimethylsilyl)oxy)spiro[4.5]decan-8-one ethylene ketal, reducing the heating time to 2 hours to prevent TBS deprotection.

### Example B12

### 2-thiaspiro[3.5]nonan-7-one

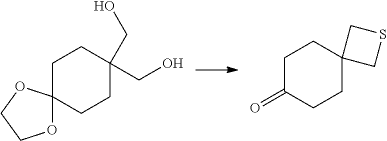 [SMILES: OCC1(CO)CCC2(CC1)OCCO2 ; O=C1CCC2(CC1)CSC2]

To a solution of 1,4-dioxaspiro[4.5]decane-8,8-diyldimethanol (0.70 g, 3.5 mmol, see Example B3) in dry pyridine (25 mL) at 0° C. was added benzenesulfonyl chloride (2.40 equiv., 1.10 mL) dropwise. The sample was warmed to room temperature and stirred overnight. The mixture was poured onto ice, then 1,4-dioxaspiro[4.5]decane-8,8-diylbis(methylene)dibenzenesulfonate (1.18 g, 2.45 mmol, 71%) was obtained by filtration.

A solution of 1,4-dioxaspiro[4.5]decane-8,8-diylbis(methylene)dibenzenesulfonate (1.16 g, 2.40 mmol) and sodium sulfide nonahydrate (0.36 equiv., 0.86 mmol, 209 mg) in dry dimethyl sulfoxide (2 mL) was heated to 90° C. and stirred for overnight. Additional sodium sulfide nonahydrate (0.36 equiv., 0.86 mmol, 209 mg) was added, and heating was continued for 3 additional days. The sample was diluted with water, extracted 3 times with EtOAc, dried over MgSO₄, filtered, and concentrated in vacuo. Purification by CombiFlash (12 g, 0-50% EtOAc in heptane, 22 min gradient) provided 2-thiaspiro[3.5]nonan-7-one (127 mg, 0.81 mmol, 33% yield).

### Example B13

### 1-methylbicyclo[4.1.0]heptan-3-one

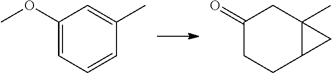

A solution of 1-methoxy-3-methylbenzene (11 g, 90.04 mmol, 1.00 equiv) in ether (60 mL) was added dropwise to liquid ammonia (150 mL) at −78° C. t-Butyl alcohol (60 mL) was added dropwise to the above solution at −78° C., then sodium (5.2 g, 226.19 mmol, 2.50 equiv) was added in portions. The resulting solution was warmed to −35° C. and stirred at −35° C. for 2 h. The resulting solution was diluted with 200 mL of pentane, quenched with 100 mL of water (carefully and very slowly), extracted with 2×100 mL of pentane, dried over anhydrous sodium sulfate, and concentrated under vacuum. This resulted in 9.2 g (82%) of 1-methoxy-5-methylcyclohexa-1,4-diene as colorless oil.

A solution of 1-methoxy-5-methylcyclohexa-1,4-diene (4.6 g, 37.04 mmol, 1.00 equiv), dichloromethane (100 mL), ethane-1,2-diol (11.5 g, 185.28 mmol, 5.00 equiv), 4-methylbenzene-1-sulfonic acid (277 mg, 1.61 mmol, 0.05 equiv) was stirred at room temperature overnight. The reaction mixture was washed with 2×50 mL of saturated sodium bicarbonate and 3×50 mL of water, dried over anhydrous sodium sulfate, and concentrated under vacuum. The residue was applied onto a silica gel column with petroleum ether. This resulted in 2.8 g (49%) of 7-methyl-1,4-dioxaspiro[4.5]dec-7-ene as colorless oil.

Trifluoroacetic acid (3.7 g, 32.45 mmol, 2.00 equiv) was added dropwise to a stirred solution of diethylzinc (1 mol/L) (33 mL, 2.00 equiv) in dichloromethane (200 mL) under nitrogen at 0° C. After 30 minutes diiodomethane (8.7 g, 32.48 mmol, 2.00 equiv) was added slowly to the reaction mixture. After another 30 minutes 7-methyl-1,4-dioxaspiro[4.5]dec-7-ene (2.5 g, 16.21 mmol, 1.00 equiv) was then added dropwise. The resulting solution was stirred for 30 min at 0° C. and quenched with 150 mL of brine. The resulting solution was extracted with 2×100 mL of dichloromethane, dried over anhydrous sodium sulfate, and concentrated under vacuum. The residue was applied onto a silica gel column with ethyl acetate/petroleum ether (1:100). This resulted in 1.1 g (40%) of 1-methylspiro[bicyclo[4.1.0]heptane-3,2-[1,3]dioxolane] as colorless oil.

A solution of 1-methylspiro[bicyclo[4.1.0]heptane-3,2-[1,3]dioxolane](1 g, 5.94 mmol, 1.00 equiv) and propan-2-one (20 mL), 4-methylbenzene-1-sulfonic acid (50 mg, 0.29 mmol, 0.05 equiv) in water (5 mL) was heated to 50° C. for 2 hours. The reaction mixture was cooled to room temperature, diluted with 200 mL of diethyl ether, washed with 1×50 mL of sodium bicarbonate and 3×50 mL of brine, dried over anhydrous sodium sulfate, and concentrated under vacuum. This resulted in 640 mg (87%) of 1-methylbicyclo[4.1.0]heptan-3-one as colorless oil.

### Example B14

### 4-(benzyloxy)-3,3-dimethylcyclohexanone

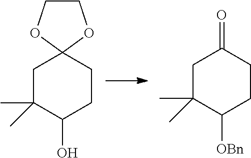 [SMILES: CC1(C)CC2(CCC1O)OCCO2 ; CC1(C)CC(=O)CCC1OCc1ccccc1]

7,7-Dimethyl-1,4-dioxaspiro[4.5]decan-8-ol (6.0 g, 32.22 mmol, 1.00 equiv; see J. Med. Chem. 2006, 49, 3421) was added dropwise to a stirred suspension of sodium hydride (2.58 g, 60% in mineral oil, 64.50 mmol, 2.00 equiv) in tetrahydrofuran (50 mL) at 0° C. After 30 minutes benzyl bromide (8.3 g, 48.53 mmol, 1.51 equiv) was added dropwise at 0° C. The resulting solution was stirred for 12 h at room temperature, quenched with water, extracted with 3×150 mL of ethyl acetate, dried over anhydrous sodium sulfate, and concentrated under vacuum. The residue was applied onto a silica gel column eluting with ethyl acetate/petroleum ether (1/20). This resulted in 8.0 g (90%) of 8-(benzyloxy)-7,7-dimethyl-1,4-dioxaspiro[4.5]decane as colorless oil.

A solution of 8-(benzyloxy)-7,7-dimethyl-1,4-dioxaspiro[4.5]decane (8.0 g, 28.95 mmol, 1.00 equiv) and p-toluene sulfonic acid (800 mg, 4.65 mmol, 0.16 equiv) in propan-2-one (150 mL)/water(30 mL) was stirred at 50° C. for 2 h. The resulting solution was diluted with 800 mL of AcOEt and washed with 3×200 mL of saturated solution of sodium bicarbonate and 1×200 mL of brine. The mixture was dried over anhydrous sodium sulfate and concentrated under vacuum. This resulted in 6.6 g (98%) of 4-(benzyloxy)-3,3-dimethylcyclohexan-1-one as colorless oil.

### Example B15a and B15b

### 7,7-dimethyloxepan-4-one and 2,2-dimethyloxepan-4-one

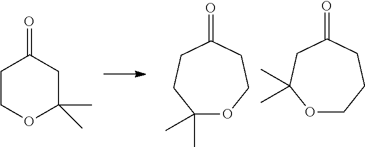

Into a 100-mL 3-necked round-bottom flask purged and maintained with nitrogen atmosphere was placed a solution of 2,2-dimethyloxan-4-one (1.3 g, 10.14 mmol, 1.00 equiv) in dichloromethane (40 mL) and boron fluoride ethyl ether (1.4 mL, 1.10 equiv). TMSCHN₂ (6 mL, 1.10 equiv, 2 mol/L in hexane) was added dropwise at −30° C. The resulting solution was stirred for 1 h at −30° C. and TLC (PE:EA=5:1) showed conversion was almost complete. The reaction was quenched with saturated sodium bicarbonate, extracted with 3×100 mL of dichloromethane. The organic layers were dried over anhydrous sodium sulfate and concentrated under vacuum. This resulted in 1.5 g of crude yellow oil as a mixture of 2,2-dimethyloxepan-4-one and 7,7-dimethyloxepan-4-one.

### Example B16

### 4-methyl-4-morpholinocyclohexanone

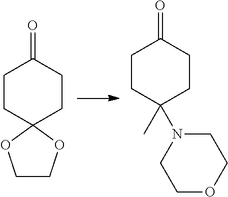

1,4-Dioxaspiro[4.5]decan-8-one (1.00 g, 6.40 mmol), 1H-triazole (1.20 equiv., 7.68), morpholine (1.10 equiv., 7.0432 mmol,), and dry toluene (30 mL) were combined, heated at 110° C. with a Dean Stark trap, and stirred overnight. The mixture was cooled to 0° C. and then methylmagnesium chloride (3 mol/L) in THF (4.00 equiv., 8.5 mL) was added dropwise. The mixture was stirred at 0° C. for 2 hours then sat NH₄Cl(aq) was added. The mixture was decanted, dried (MgSO₄), filtered, and concentrated in vacuo. Purification by CombiFlash (40 g, 0-20% EtOAc in heptane, 28 min gradient) provided 0.43 g (28%) of the 4-(8-methyl-1,4-dioxaspiro[4.5]decan-8-yl)morpholine.

4-(8-Methyl-1,4-dioxaspiro[4.5]decan-8-yl)morpholine (0.458 g, 1.898 mmol), glacial acetic acid (5 mL), and water (5 mL) were combined and the mixture was heated at 65° C. overnight. The mixture was concentrated in vacuo, diluted with sat NaHCO₃(aq), extracted 9 times with 10% MeOH in dichloromethane, dried (MgSO₄), and concentrated in vacuo. Due to incomplete deprotection, the mixture was diluted with water (5 mL) cooled to 0° C., then hydrochloric acid (7.0 M, 8.00 equiv., 2 mL) was added dropwise. The mixture was allowed to warm slowly to room temperature and stirred for 3 days. The mixture was cooled to 0° C., then 50% NaOH was added until pH9. The mixture was then extracted 3 times with 10% MeOH in dichloromethane, dried (MgSO₄), and concentrated in vacuo. Purification by CombiFlash (12 g, 0-100% EtOAc in heptane, 11 min gradient) provided 0.303 g (81%) of 4-methyl-4-morpholinocyclohexanone.

### Example B17

### 4-methyl-4-morpholinocyclohexanone

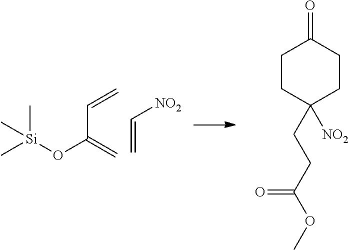

Trimethyl(1-methyleneallyloxy)silane (2.00 g, 14.1 mmol) and 2.0 M 1-nitroethylene in toluene (14.1 mmol, 7.05 mL) were combined and heated at 80° C. overnight. The mixture was filtered from the insoluble solids and concentrated in vacuo. Purification by CombiFlash (40 g, 0-40% EtOAc in heptane, 28 min gradient) provided 1.03 g (51%) of 4-nitrocyclohexanone.

4-Nitrocyclohexanone (0.300 g, 2.10 mmol), methyl acrylate (2.52 mmol), 1,1,3,3-tetramethylguanidine (0.0541 equiv., 0.113 mmol), and acetonitrile (0.5 mL) were combined and stirred for 3 days. The mixture was concentrated in vacuo. Purification by CombiFlash (12 g, 0-50% EtOAc in heptane, 11 min gradient) provided 0.40 g (83%) of the title compound.

### Example B18

### 7,7-difluoro-1-methylbicyclo[4.1.0]heptan-3-one

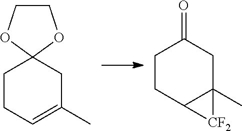

Into a 100-mL sealed tube purged and maintained with an inert atmosphere of nitrogen, was placed NaI (680 mg, 4.53 mmol, 0.50 equiv), tetrahydrofuran (28 mL), 7-methyl-1,4-dioxaspiro[4.5]dec-7-ene (1.4 g, 9.08 mmol, 1.00 equiv; see Example B13), and TMSCF₃ (3.23 g, 22.75 mmol, 2.51 equiv). The reaction was stirred for 12 h at 65° C. and then quenched with 20 mL of water. The resulting solution was extracted with ethyl acetate, washed with saturated Na₂S₂CO₃ and brine, dried over anhydrous sodium sulfate, and concentrated under vacuum. The residue was applied onto a silica gel column eluting with ethyl acetate/petroleum ether (1/100). This resulted in 1.6 g (86%) of 7,7-difluoro-1-methylspiro[bicyclo[4.1.0]heptane-3,2-[1,3]dioxolane] as a colorless oil.

A solution of 7,7-difluoro-1-methylspiro[bicyclo[4.1.0]heptane-3,2-[1,3]dioxolane](1.6 g, 7.83 mmol, 1.00 equiv) and PTSA (135 mg, 0.78 mmol, 0.10 equiv) in acetone (25 mL)/water (5 mL) was stirred for 12 h at 50° C. The reaction mixture was diluted with 300 mL of diethyl ether, washed with saturated sodium bicarbonate and brine, dried over anhydrous sodium sulfate, and concentrated under vacuum. This resulted in 1.1 g (88%) of 7,7-difluoro-1-methylbicyclo[4.1.0]heptan-3-one as a light yellow oil.

### Synthesis of Pyrazole Carboxylates

### Examples C

### Example C1

### 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid

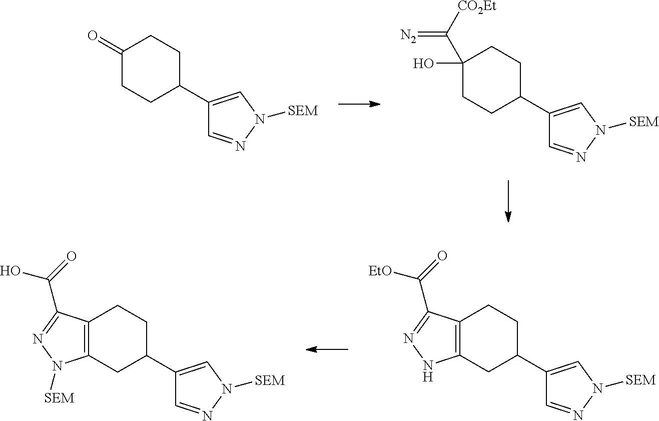

Step 1:

A solution of diisopropylamine (0.503 mL, 3.57 mmol) in THF (10 mL) was cooled to −78° C., then a solution of n-butyl lithium in hexanes (1.6 M, 2.00 mL, 3.20 mmol) was added dropwise. After stirring for 5 minutes, this mixture was added via cannula to a −78° C. solution of ethyl diazoacetate (0.355 mL, 3.36 mmol) and 4-(1-((2-(trimethylsilyl)ethoxy)methyl)-1H-pyrazol-4-yl)cyclohexanone (Example B1, 619 mg, 2.10 mmol) in THF (10 mL). The mixture was stirred for 1 hour at −78° C., then quenched by the addition of sat. NH₄Cl(aq). The mixture was diluted with water and extracted with EtOAc (2 times). The combined organic extracts were dried (MgSO₄) and concentrated in vacuo. Purification by CombiFlash (40 g; 100:0 to 70:30 heptane:EtOAc) provided 810 mg (1.97 mmol) of ethyl 2-diazo-2-(1-hydroxy-4-(1-((2-(trimethylsilyl)ethoxy)methyl)-1H-pyrazol-4-yl)cyclohexyl)acetate as a mixture of diastereomers.

Step 2:

To a solution of ethyl 2-diazo-2-(1-hydroxy-4-(1-((2-(trimethylsilyl)ethoxy)methyl)-1H-pyrazol-4-yl)cyclohexyl)acetate (810 mg, 1.97 mmol) in pyridine (8 mL) was added POCl₃ (0.743 mL, 7.89 mmol) and the mixture was allowed to stir at room temperature overnight. After in vacuo concentration, the mixture was poured onto ice, then extracted with EtOAc (3 times). The combined organic extracts were dried (MgSO₄) and concentrated in vacuo. This residue was diluted with octane (4 mL) and heated to 110° C. overnight. After in vacuo concentration, purification by CombiFlash (12 g; 100:0 to 0:100 heptane:EtOAc) provided 418 mg (1.07 mmol) of ethyl 6-(1-((2-(trimethylsilyl)ethoxy)methyl)-1H-pyrazol-4-yl)-4,5,6,7-tetrahydro-1H-indazole-3-carboxylate.

Step 3:

A solution of ethyl 6-(1-((2-(trimethylsilyl)ethoxy)methyl)-1H-pyrazol-4-yl)-4,5,6,7-tetrahydro-1H-indazole-3-carboxylate (418 mg, 1.07 mmol) in THF (21 mL) was cooled to 0° C., then sodium hydride (60%, 128 mg, 3.21 mmol) was added. After stirring for 1 hour, SEMCl (0.227 mL, 1.28 mmol) was added and the mixture was allowed to warm to room temperature overnight. After excess hydride was quenched by the addition of water at 0° C., the mixture was extracted with EtOAc (3 times), the organic extracts dried (MgSO₄) and concentrated in vacuo. Purification by CombiFlash (40 g; 100:0 to 50:50 heptane:EtOAc) provided 504 mg (0.967 mmol) of ethyl 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylate. This ester was diluted with THF (6 mL), acetonitrile (6 mL) and water (6 mL) and lithium hydroxide monohydrate (328 mg, 7.74 mmol) was added and the mixture was stirred overnight. The mixture was diluted with water, acidified to pH 3 with 1 N HCl(aq) and extracted with Et₂O (once) and 10% MeOH/CH₂Cl₂ (3 times). The combined organic extracts were dried (MgSO₄) and concentrated in vacuo to provide 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid of sufficient purity to be used directly (448 mg, 0.911 mmol).

### Example C2

### 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid

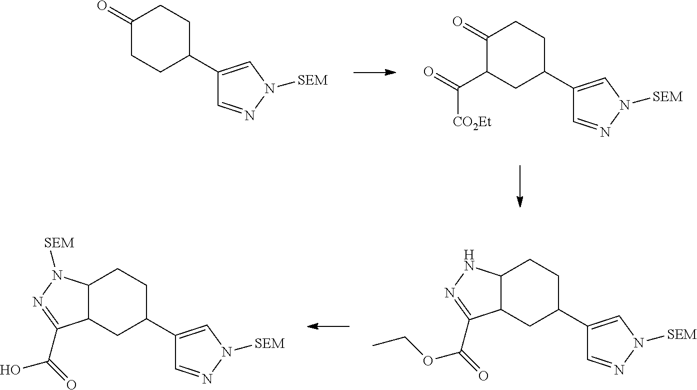 [SMILES: C[Si](C)(C)CCOCn1cc(C2CCC(=O)CC2)cn1 ; CCOC(=O)C(=O)C1CC(c2cnn(COCC[Si](C)(C)C)c2)CCC1=O ; C[Si](C)(C)CCOCN1N=C(C(=O)O)C2CC(c3cnn(COCC[Si](C)(C)C)c3)CCC21 ; CCOC(=O)C1=NNC2CCC(c3cnn(COCC[Si](C)(C)C)c3)CC12]

Step 1:

A solution of 4-(1-((2-(trimethylsilyl)ethoxy)methyl)-1H-pyrazol-4-yl)cyclohexanone (Example B1, 424 mg, 1.44 mmol) in EtOH (0.7 mL) was cooled to 0° C., then sodium ethoxide (21% wt solution in EtOH, 0.592 mL, 1.58 mmol) was added. To this mixture was added diethyl oxylate (0.195 mL, 1.44 mmol) and the mixture was allowed to warm to room temperature overnight. In vacuo concentration provided ethyl 2-oxo-2-(2-oxo-5-(1-((2-(trimethylsilyl)ethoxy)methyl)-1H-pyrazol-4-yl)cyclohexyl)acetate of sufficient purity to be used directly (yield assumed to be quantitative).

Step 2:

A solution of ethyl 2-oxo-2-(2-oxo-5-(1-((2-(trimethylsilyl)ethoxy)methyl)-1H-pyrazol-4-yl)cyclohexyl)acetate (568 mg, unpurified) in glacial acetic acid (0.7 mL) was cooled to 0° C., then hydrazine hydrate (0.120 mL, 1.58 mmol) was added. After warming to room temperature, the mixture was stirred for 1 hour, then diluted with sat. NaHCO₃(aq) and extracted with 10% MeOH/CH₂Cl₂. The organic extracts were dried (MgSO₄) and concentrated in vacuo. Purification by CombiFlash (12 g; 100:0 to 50:50 heptane:EtOAc) provided 229 mg (0.587 mmol) of ethyl 5-(1-((2-(trimethylsilyl)ethoxy)methyl)-1H-pyrazol-4-yl)-4,5,6,7-tetrahydro-1H-indazole-3-carboxylate.

Step 3:

Performed in an analogous manner to Step 3 for 1-((2-(trimethylsilyl)ethoxy)methyl)-6-(1-((2-(trimethylsilyl)ethoxy)methyl)-1-pyrazol-4-yl)-4,5,6,7-tetrahydro-1H-indazole-3-carboxylic acid (Example C1), replacing methyl 6-(1-((2-(trimethylsilyl)ethoxy)methyl)-1H-pyrazol-4-yl)-4,5,6,7-tetrahydro-1H-indazole-3-carboxylate with ethyl 5-(1-((2-(trimethylsilyl)ethoxy)methyl)-1H-pyrazol-4-yl)-4,5,6,7-tetrahydro-1H-indazole-3-carboxylate.

### Example C3

### 1-((2-(trimethylsilyl)ethoxy)methyl)-4,5,6,7-tetrahydro-1H-indazole-3-carboxylic acid

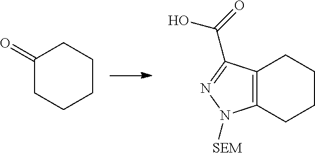 [SMILES: O=C1CCCCC1 ; C[Si](C)(C)CCOCn1nc(C(=O)O)c2c1CCCC2]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with cyclohexanone (commercial).

### Example C4

### 6-(pyrimidin-5-yl)-1-((2-(trimethylsilyl)ethoxy)methyl)-4,5,6,7-tetrahydro-1H-indazole-3-carboxylic acid

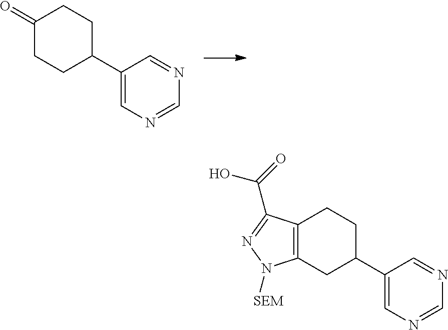 [SMILES: O=C1CCC(c2cncnc2)CC1 ; C[Si](C)(C)CCOCn1nc(C(=O)O)c2c1CC(c1cncnc1)CC2]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4-(pyrimidin-5-yl)cyclohexanone (Example B2).

### Example C5

### 6-(tert-butyldimethylsilyloxy)-1-((2-(trimethylsilyl)ethoxy)methyl)-4,5,6,7-tetrahydro-1H-indazole-3-carboxylic acid

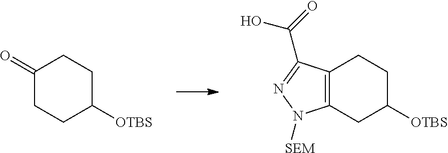 [SMILES: CC(C)(C)[Si](C)(C)OC1CCC(=O)CC1 ; CC(C)(C)[Si](C)(C)OC1CCc2c(C(=O)O)nn(COCC[Si](C)(C)C)c2C1]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4-(tert-butyldimethylsilyloxy)cyclohexanone (commercial).

### Example C6

### 6,6-dimethyl-1-((2-(trimethylsilyl)ethoxy)methyl)-4,5,6,7-tetrahydro-1H-indazole-3-carboxylic acid

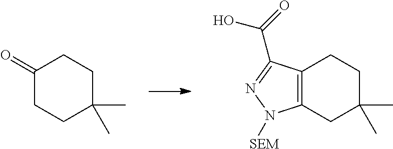

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4,4-dimethylcyclohexanone (commercial).

### Example C7

### 5,5-dimethyl-1-((2-(trimethylsilyl)ethoxy)methyl)-4,5,6,7-tetrahydro-1H-indazole-3-carboxylic

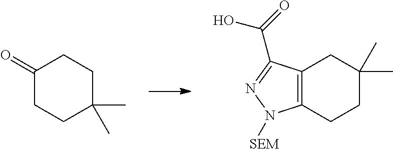

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 4,4-dimethylcyclohexanone (commercial).

### Example C8

### 5-methyl-1-((2-(trimethylsilyl)ethoxy)methyl)-4,5,6,7-tetrahydro-1H-indazole-3-carboxylic acid

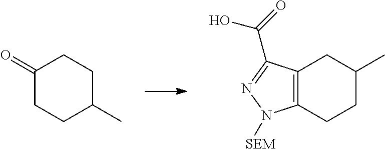

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 4-methylcyclohexanone (commercial).

### Example C9

### 6-methyl-1-((2-(trimethylsilyl)ethoxy)methyl)-4,5,6,7-tetrahydro-1H-indazole-3-carboxylic acid

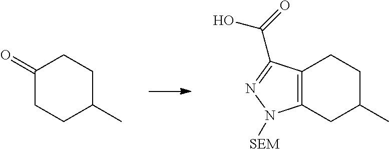

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4-methylcyclohexanone (commercial).

### Example C10

### 1′-((2-(trimethylsilyl)ethoxy)methyl)-1′,4′,5′,7′-tetrahydrospiro[cyclopropane-1,6′-indazole]-3′-carboxylic acid

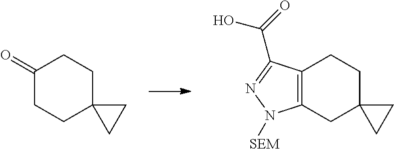 [SMILES: O=C1CCC2(CC1)CC2 ; C[Si](C)(C)CCOCn1nc(C(=O)O)c2c1CC1(CC2)CC1]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with spiro[2.5]octan-6-one (commercial).

### Example C11

### 1-((2-(trimethylsilyl)ethoxy)methyl)-1,4,6,7-tetrahydrospiro[indazole-5,3-oxetane]-3-carboxylic acid

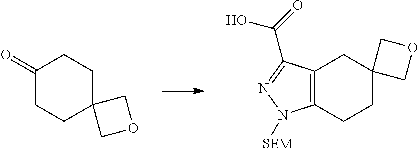 [SMILES: O=C1CCC2(CC1)COC2 ; C[Si](C)(C)CCOCn1nc(C(=O)O)c2c1CCC1(COC1)C2]

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 2-oxaspiro[3.5]nonan-7-one (Example B3).

### Example C12

### 1-((2-(trimethylsilyl)ethoxy)methyl)-1,4,5,7-tetrahydrospiro[indazole-6,3′-oxetane]-3-carboxylic acid

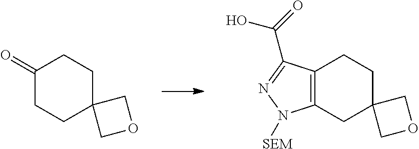 [SMILES: O=C1CCC2(CC1)COC2 ; C[Si](C)(C)CCOCn1nc(C(=O)O)c2c1CC1(CC2)COC1]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 2-oxaspiro[3.5]nonan-7-one (Example B3). Also the dehydration step (Step 2) was performed using modified conditions as follows:

To a solution of ethyl 2-diazo-2-(7-hydroxy-2-oxaspiro[3.5]nonan-7-yl)acetate (100 mg, 0.393 mmol) in CH₂Cl₂ (2.5 mL) was added triethylamine (0.138 mL, 0.983 mmol) and trifluoroacetic anhydride (0.111 mL, 0.787 mmol). The mixture was stirred for 15 minutes, then diluted with H₂O and extracted with CH₂Cl₂. The combined organic extracts were dried (MgSO₄) and concentrated in vacuo. The residue was used directly in Step 3 without purification.

### Example C13

### 6-((tert-butyldimethylsilyloxy)methyl)-1-((2-(trimethylsilyl)ethoxy)methyl)-4,5,6,7-tetrahydro-1H-indazole-3-carboxylic acid

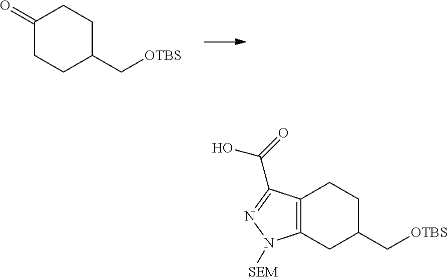 [SMILES: CC(C)(C)[Si](C)(C)OCC1CCC(=O)CC1 ; CC(C)(C)[Si](C)(C)OCC1CCc2c(C(=O)O)nn(COCC[Si](C)(C)C)c2C1]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4-((tert-butyldimethylsilyloxy)methyl)cyclohexanone (see J. Org. Chem. 2005, 70, 2409).

### Example C14

### 5,5-difluoro-1-((2-(trimethylsilyl)ethoxy)methyl)-4,5,6,7-tetrahydro-1H-indazole-3-carboxylic acid

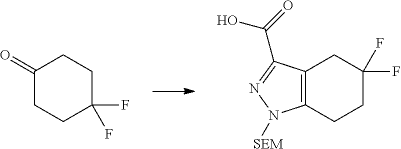 [SMILES: O=C1CCC(F)(F)CC1 ; C[Si](C)(C)CCOCn1nc(C(=O)O)c2c1CCC(F)(F)C2]

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 4,4-difluorocyclohexanone (commercial).

### Example C15

### 6,6-difluoro-1-((2-(trimethylsilyl)ethoxy)methyl)-4,5,6,7-tetrahydro-1H-indazole-3-carboxylic acid

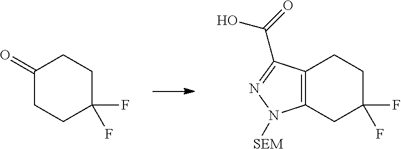 [SMILES: O=C1CCC(F)(F)CC1 ; C[Si](C)(C)CCOCn1nc(C(=O)O)c2c1CC(F)(F)CC2]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4,4-difluorocyclohexanone (commercial).

### Example C16

### 6-((tert-butyldimethylsilyloxy)methyl)-6-methyl-1-((2-(trimethylsilyl)ethoxy)methyl)-4,5,6,7-tetrahydro-1H-indazole-3-carboxylic acid

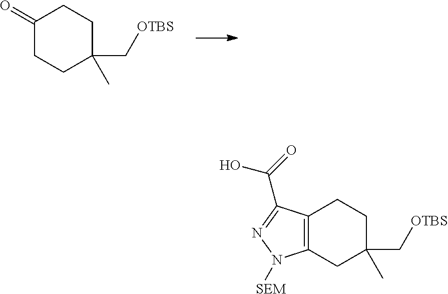 [SMILES: CC1(CO[Si](C)(C)C(C)(C)C)CCC(=O)CC1 ; CC1(CO[Si](C)(C)C(C)(C)C)CCc2c(C(=O)O)nn(COCC[Si](C)(C)C)c2C1]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4-((tert-butyldimethylsilyloxy)methyl)-4-methylcyclohexanone (Example B4).

### Example C17

### 1-((2-(trimethylsilyl)ethoxy)methyl)-1,4,5,6-tetrahydrocyclopenta[c]pyrazole-3-carboxylic acid

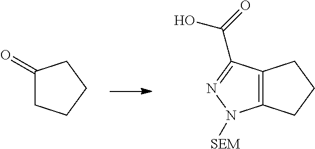 [SMILES: O=C1CCCC1 ; C[Si](C)(C)CCOCn1nc(C(=O)O)c2c1CCC2]

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with cyclopentaone (commercial).

### Example C18

### 5,5-dimethyl-1-((2-(trimethylsilyl)ethoxy)methyl)-1,4,5,6-tetrahydrocyclopenta[c]pyrazole-3-carboxylic acid

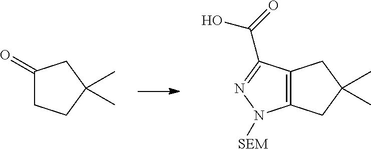

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 3,3-dimethylcyclopentaone (commercial).

### Examples C19a and C19b

### 1-((2-(trimethylsilyl)ethoxy)methyl)-1,4,4a,5,5a,6-hexahydrocyclopropa[f]indazole-3-carboxylic acid and 3-((2-(trimethylsilyl)ethoxy)methyl)-3,4,5,5a,6,6a-hexahydrocyclopropa[e]indazole-1-carboxylic acid

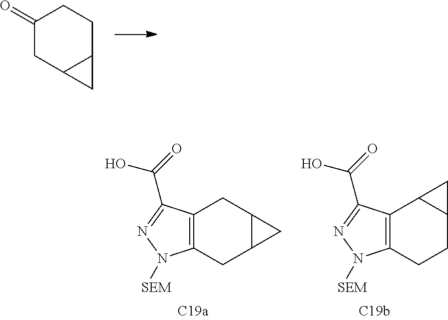 [SMILES: O=C1CCC2CC2C1 ; C[Si](C)(C)CCOCn1nc(C(=O)O)c2c1CC1CC1C2 ; C[Si](C)(C)CCOCn1nc(C(=O)O)c2c1CCC1CC21]

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with bicyclo[4.1.0]heptan-3-one (see J. Am. Chem. Soc. 1968, 90, 6406). The regioisomers were separated by preparative HPLC.

### Example C20

### 6-methoxy-6-methyl-1-((2-(trimethylsilyl)ethoxy)methyl)-4,5,6,7-tetrahydro-1H-indazole-3-carboxylic acid

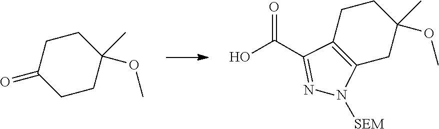

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4-methoxy-4-methylcyclohexanone (see US2009/29977 A1).

### Example C21

### 6-((tert-butyldimethylsilyl)oxy)-6-methyl-1-((2-(trimethylsilyl)ethoxy)methyl)-4,5,6,7-tetrahydro-1H-indazole-3-carboxylic acid

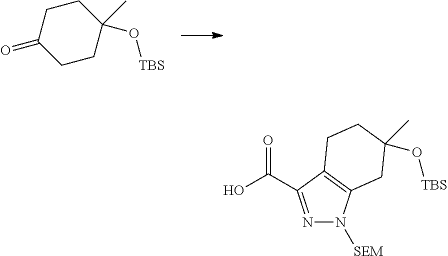

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4-[tert-butyl(dimethyl)silyl]oxy-4-methyl-cyclohexanone (Example B5).

### Example C22

### 6-ethyl-6-methyl-1-((2-(trimethylsilyl)ethoxy)methyl)-4,5,6,7-tetrahydro-1H-indazole-3-carboxylic acid

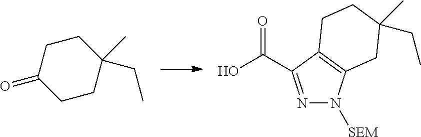

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4-ethyl-4-methyl-cyclohexanone (Example B6).

### Example C23

### 1′-((2-(trimethylsilyl)ethoxy)methyl)-1,4,4′,5,5′,7′-hexahydro-3H-spiro[furan-2,6′-indazole]-3′-carboxylic acid

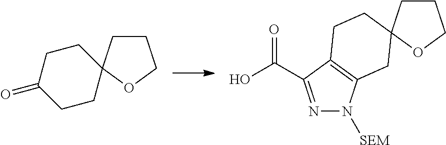 [SMILES: O=C1CCC2(CCCO2)CC1 ; C[Si](C)(C)CCOCn1nc(C(=O)O)c2c1CC1(CCCO1)CC2]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 1-oxaspiro[4.5]decan-8-one (see US2011/263424 A1).

### Example C24

### 1′-((2-(trimethylsilyl)ethoxy)methyl)-1,4,4′,5,5′,7′-hexahydro-2H-spiro[furan-3,6-indazole]-3′-carboxylic acid

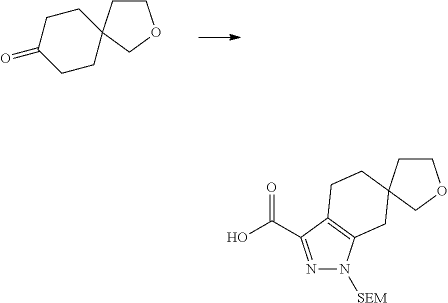 [SMILES: O=C1CCC2(CCOC2)CC1 ; C[Si](C)(C)CCOCn1nc(C(=O)O)c2c1CC1(CCOC1)CC2]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 2-oxaspiro[4.5]decan-8-one (Example B7).

### Example C25

### 1′-methyl-2′-oxo-1-((2-(trimethylsilyl)ethoxy)methyl)-1,4,5,7-tetrahydrospiro[indazole-6,3′-pyrrolidine]-3-carboxylic acid

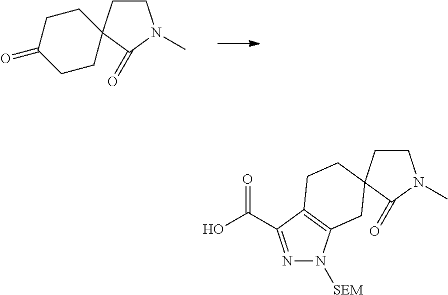 [SMILES: CN1CCC2(CCC(=O)CC2)C1=O ; CN1CCC2(CCc3c(C(=O)O)nn(COCC[Si](C)(C)C)c3C2)C1=O]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 2-methyl-2-azaspiro[4.5]decane-1,8-dione (Example B8).

### Example C26

### 1-((2-(trimethylsilyl)ethoxy)methyl)-4,4′,5′,6-tetrahydro-1H,2′H-spiro[cyclopenta[c]pyrazole-5,3′-furan]-3-carboxylic acid

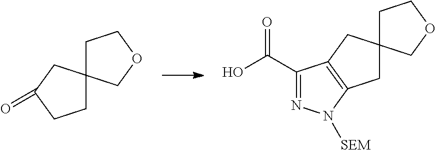 [SMILES: O=C1CCC2(CCOC2)C1 ; C[Si](C)(C)CCOCn1nc(C(=O)O)c2c1CC1(CCOC1)C2]

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 2-oxaspiro[4.4]nonan-7-one (Example B9).

### Example C27

### 5-cyano-6,6-dimethyl-1-((2-(trimethylsilyl)ethoxy)methyl)-4,5,6,7-tetrahydro-1H-indazole-3-carboxylic acid

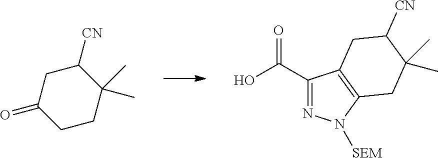 [SMILES: CC1(C)CCC(=O)CC1C#N ; CC1(C)Cc2c(c(C(=O)O)nn2COCC[Si](C)(C)C)CC1C#N]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 2,2-dimethyl-5-oxocyclohexanecarbonitrile (see Can. J. Chem. 2000, 78, 925). NOTE: a mixture of regioisomeric products is obtained by this process, and the desired title compound is separated by chromatography and assigned by 2D NMR.

### Example C28

### 1-((2-(trimethylsilyl)ethoxy)methyl)-4,6-dihydro-1H-spiro[cyclopenta[c]pyrazole-5,3′-oxetane]-3-carboxylic acid

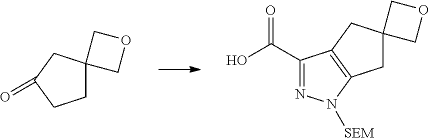 [SMILES: O=C1CCC2(COC2)C1 ; C[Si](C)(C)CCOCn1nc(C(=O)O)c2c1CC1(COC1)C2]

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 2-oxaspiro[3.4]octan-6-one (Example B10).

### Example C29

### 1-((2-(trimethylsilyl)ethoxy)methyl)-1,4,5,7-tetrahydrospiro[indazole-6,3′-thietane]-3-carboxylic acid

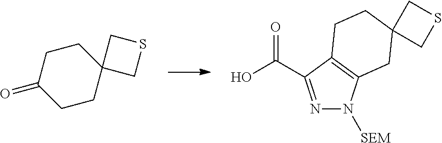 [SMILES: O=C1CCC2(CC1)CSC2 ; C[Si](C)(C)CCOCn1nc(C(=O)O)c2c1CC1(CC2)CSC1]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 2-thiaspiro[3.5]nonan-7-one (Example B12).

### Example C30

### 2-((tert-butyldimethylsilyl)oxy)-1′-((2-(trimethylsilyl)ethoxy)methyl)-1′,4′,5′,7′-tetrahydrospiro[cyclopentane-1,6′-indazole]-3′-carboxylic acid

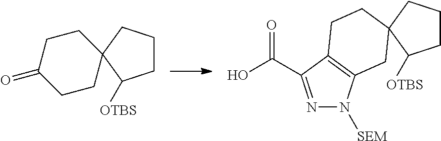 [SMILES: CC(C)(C)[Si](C)(C)OC1CCCC12CCC(=O)CC2 ; CC(C)(C)[Si](C)(C)OC1CCCC12CCc1c(C(=O)O)nn(COCC[Si](C)(C)C)c1C2]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 1-((tert-butyldimethylsilyl)oxy)spiro[4.5]decan-8-one (Example B11).

### Example C31

### 5a-methyl-1-((2-(trimethylsilyl)ethoxy)methyl)-1,4,4a,5,5a,6-hexahydrocyclopropa[f]indazole-3-carboxylic acid

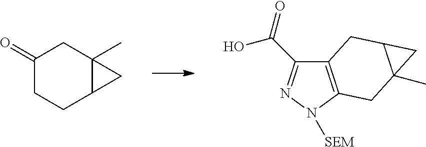

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 1-methylbicyclo[4.1.0]heptan-3-one (Example B13).

### Example C31a

### 5a-methyl-1-((2-(trimethylsilyl)ethoxy)methyl)-1,4,4a,5,5a,6-hexahydrocyclopropa[f]indazole-3-carboxylic acid

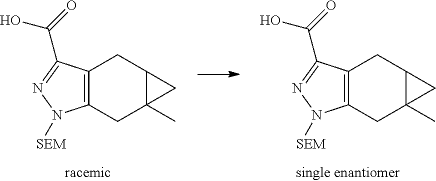 [SMILES: CC12Cc3c(c(C(=O)O)nn3COCC[Si](C)(C)C)CC1C2 ; CC12Cc3c(c(C(=O)O)nn3COCC[Si](C)(C)C)CC1C2]

Racemic 5a-methyl-1-((2-(trimethylsilyl)ethoxy)methyl)-1,4,4a,5,5a,6-hexahydrocyclopropa[f]indazole-3-carboxylic acid (Example C31) was resolved into its constituent enantiomers by SFC with a chiral stationary phase. The two enantiomers were carried separately forward to Examples 29a and 29b, and the enantiomeric starting material which provided Example 29b was used for subsequent transformations as Example C31a.

### Example C32

### 1-((2-(trimethylsilyl)ethoxy)methyl)-4,5,6,7-tetrahydro-1H-4,6-methanoindazole-3-carboxylic acid

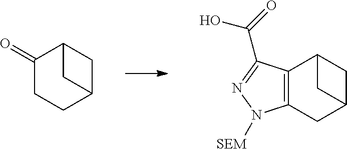 [SMILES: O=C1CCC2CC1C2 ; C[Si](C)(C)CCOCn1nc(C(=O)O)c2c1CC1CC2C1]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with bicyclo[3.1.1]heptan-2-one (see J. Am. Chem. Soc. 1980, 102, 1404).

### Example C33

### 1-((2-(trimethylsilyl)ethoxy)methyl)-4,5,6,7-tetrahydro-1H-5,7-methanoindazole-3-carboxylic acid

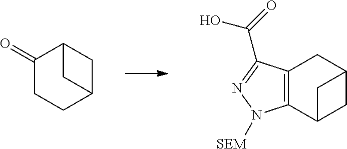 [SMILES: O=C1CCC2CC1C2 ; C[Si](C)(C)CCOCn1nc(C(=O)O)c2c1C1CC(C2)C1]

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with bicyclo[3.1.1]heptan-2-one (see J. Am. Chem. Soc. 1980, 102, 1404).

### Example C34

### 5-(benzyloxy)-6,6-dimethyl-1-((2-(trimethylsilyl)ethoxy)methyl)-4,5,6,7-tetrahydro-1H-indazole-3-carboxylic acid

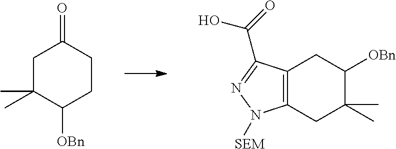 [SMILES: CC1(C)CC(=O)CCC1OCc1ccccc1 ; CC1(C)Cc2c(c(C(=O)O)nn2COCC[Si](C)(C)C)CC1OCc1ccccc1]

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 4-(benzyloxy)-3,3-dimethylcyclohexan-1-one one (Example B14).

### Example C35a and C35b

### 7,7-dimethyl-1-((2-(trimethylsilyl)ethoxy)methyl)-4,5,7,8-tetrahydro-1H-oxepino[4,5-c]pyrazole-3-carboxylic acid and 5,5-dimethyl-1-((2-(trimethylsilyl)ethoxy)methyl)-4,5,7,8-tetrahydro-1H-oxepino[4,5-c]pyrazole-3-carboxylic acid

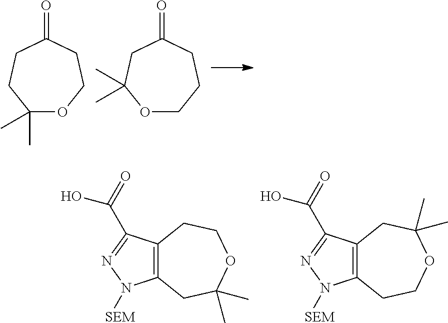

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with the mixture of 2,2-dimethyloxepan-4-one and 7,7-dimethyloxepan-4-one (Example B15a/b).

### Example C36

### 6-cyano-6-methyl-1-((2-(trimethylsilyl)ethoxy)methyl)-4,5,6,7-tetrahydro-1H-indazole-3-carboxylic acid

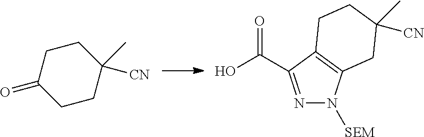

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 1-methyl-4-oxocyclohexanecarbonitrile (see WO2009/156099 A1).

### Example C37

### 5,5-difluoro-5a-methyl-1,4,4a,5,5a,6-hexahydrocyclopropa[f]indazole-3-carboxylic acid

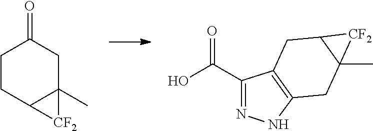 [SMILES: CC12CC(=O)CCC1C2(F)F ; CC12Cc3[nH]nc(C(=O)O)c3CC1C2(F)F]

Under nitrogen t-BuOK (3.1 mL, 1M in THF, 1.00 equiv) was added dropwise into a solution of 7,7-difluoro-1-methylbicyclo[4.1.0]heptan-3-one (500 mg, 3.12 mmol, 1.00 equiv; Example B18) and diethyl oxalate (456 mg, 3.12 mmol, 1.00 equiv) in tetrahydrofuran (10 mL) at −70° C. The reaction mixture was stirred for 12 h at −70° C., quenched by 5 mL of saturated NH₄Cl, washed with brine, dried over anhydrous sodium sulfate, and concentrated under vacuum. This resulted in 620 mg (76%) of ethyl 2-[7,7-difluoro-6-methyl-4-oxobicyclo[4.1.0]heptan-3-yl]-2-oxoacetate as a brown oil.

A solution of hydrazine hydrate (763 mg, 15.24 mmol, 6.40 equiv), ethyl 2-[7,7-difluoro-6-methyl-4-oxobicyclo[4.1.0]heptan-3-yl]-2-oxoacetate (620 mg, 2.38 mmol, 1.00 equiv) in acetic acid (15 mL) was stirred for 12 h at 120° C. The reaction was cooled to room temperature and the pH value of the solution was adjusted to 8 to 9 with saturated sodium bicarbonate. The resulting solution was extracted with ethyl acetate, washed with brine, dried over anhydrous sodium sulfate, and concentrated under vacuum. The residue was applied onto a silica gel column eluting with ethyl acetate/petroleum ether (1/2). This resulted in 300 mg (49%) of ethyl 5,5-difluoro-5a-methyl-1,4,4a,5,5a,6-hexahydrocyclopropa[f]indazole-3-carboxylate.

A solution of ethyl 5,5-difluoro-5a-methyl-1,4,4a,5,5a,6-hexahydrocyclopropa[f]indazole-3-carboxylate (300 mg, 1.17 mmol, 1.00 equiv), ethanol (12 mL), water (2.4 mL), and sodium hydroxide (469 mg, 11.72 mmol, 10.02 equiv) was stirred for 2 h at 50° C. The reaction mixture was concentrated under vacuum and the residue was dissolved in 50 mL of water. The pH value of the solution was adjusted to 4 to 5 with 1 N of hydrogen chloride. The solid was collected by filtration and dried under vacuum to provide 250 mg (94%) of 5,5-difluoro-5a-methyl-1,4,4a,5,5a,6-hexahydrocyclopropa[f]indazole-3-carboxylic acid.

### Example C38

### 5′-oxo-1-((2-(trimethylsilyl)ethoxy)methyl)-1,4,5,7-tetrahydrospiro[indazole-6,2′-pyrrolidine]-3-carboxylic acid

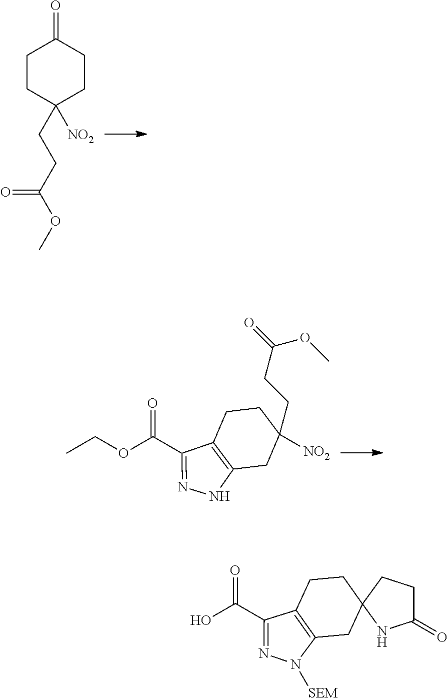 [SMILES: COC(=O)CCC1([N+](=O)[O-])CCC(=O)CC1 ; CCOC(=O)c1n[nH]c2c1CCC(CCC(=O)OC)([N+](=O)[O-])C2 ; C[Si](C)(C)CCOCn1nc(C(=O)O)c2c1CC1(CCC(=O)N1)CC2]

Ethyl 6-(3-methoxy-3-oxopropyl)-6-nitro-4,5,6,7-tetrahydro-1H-indazole-3-carboxylate was prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B) with methyl 3-(1-nitro-4-oxocyclohexyl)propanoate (Example B17), and not performing Step 3.

Ethyl 6-(3-methoxy-3-oxopropyl)-6-nitro-4,5,6,7-tetrahydro-1H-indazole-3-carboxylate (0.2261 g, 0.6951 mmol) in dry tetrahydrofuran (62 mmol) at 0° C. was added 60% sodium hydride in oil (3.00 equiv., 2.085 mmol). The mixture was stirred at 0° C. for 30 min then added 2-(chloromethoxy)ethyl-trimethyl-silane (1.20 equiv., 0.8341 mmol) dropwise. The mixture was stirred at 0° C. for 30 min, then H₂O was added dropwise to the mixture. The mixture was extracted 3 times with EtOAc, dried (MgSO₄), and concentrated in vacuo. Purification by CombiFlash (12 g, 0-50% EtOAc in heptane) provided 0.2244 g (71%) of ethyl 6-(3-methoxy-3-oxopropyl)-6-nitro-1-((2-(trimethylsilyl)ethoxy)methyl)-4,5,6,7-tetrahydro-1H-indazole-3-carboxylate.

Ethyl 6-(3-methoxy-3-oxopropyl)-6-nitro-1-((2-(trimethylsilyl)ethoxy)methyl)-4,5,6,7-tetrahydro-1H-indazole-3-carboxylate (0.2244 g, 0.4925 mmol), ammonium formate (5.00 equiv., 2.463 mmol, 100 mass %), 10% palladium on carbon (0.100 g) and dry methanol (44 mmol) were combined under nitrogen, purged with hydrogen, heated at 65° C., and stirred overnight under an atmosphere of hydrogen. The mixture was purged with nitrogen, added celite, filtered through a pad of celite, and concentrated in vacuo. Dry ethanol (44 mmol) was added to the mixture, which was heated at 78° C. overnight. The mixture was concentrated in vacuo. Purification by CombiFlash (4 g, 0-10% MeOH in dichloromethane, 22 min gradient) provided 0.139 g (72%) of ethyl 5′-oxo-1-((2-(trimethylsilyl)ethoxy)methyl)-1,4,5,7-tetrahydrospiro[indazole-6,2′-pyrrolidine]-3-carboxylate.

Ester hydrolysis was accomplished in an analogous manner to the final step of 5,5-difluoro-5a-methyl-1,4,4a,5,5a,6-hexahydrocyclopropa[f]indazole-3-carboxylic acid (Example C37), replacing ethyl 5,5-difluoro-5a-methyl-1,4,4a,5,5a,6-hexahydrocyclopropa[f]indazole-3-carboxylate with ethyl 5′-oxo-1-((2-(trimethylsilyl)ethoxy)methyl)-1,4,5,7-tetrahydrospiro[indazole-6,2′-pyrrolidine]-3-carboxylate.

### Example C39

### 5a-methyl-1-((2-(trimethylsilyl)ethoxy)methyl)-1,4,5,5a,6,6a-hexahydrocyclopropa[g]indazole-3-carboxylic acid

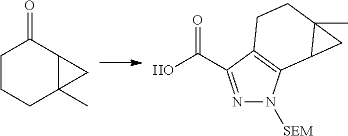

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 6-methylbicyclo[4.1.0]heptan-2-one (J. Org. Chem. 1996, 61, 8885).

### Example C40

### 4a-methyl-1-((2-(trimethylsilyl)ethoxy)methyl)-4,4a,5,5a-tetrahydro-1H-cyclopropa[4,5]cyclopenta[1,2-c]pyrazole-3-carboxylic acid

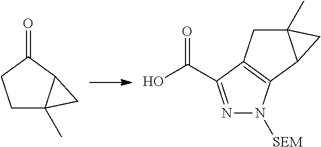

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with 5-methylbicyclo[3.1.0]hexan-2-one (J. Org. Chem. 1996, 61, 8885).

### Example C41

### 4a-methyl-1-((2-(trimethylsilyl)ethoxy)methyl)-3b,4,4a,5-tetrahydro-1H-cyclopropa[3,4]cyclopenta[1,2-c]pyrazole-3-carboxylic acid

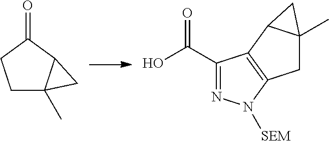

Prepared in an analogous manner to 1-((2-(trimethylsilyl)ethoxy)methyl)-6-(1-((2-(trimethylsilyl)ethoxy)methyl)-1-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 5-methylbicyclo[3.1.0]hexan-2-one (J. Org. Chem. 1996, 61, 8885).

### Example C42

### 1-((2-(trimethylsilyl)ethoxy)methyl)-1,4,5,6,7,8-hexahydrocyclohepta[c]pyrazole-3-carboxylic acid

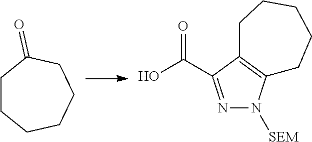 [SMILES: O=C1CCCCCC1 ; C[Si](C)(C)CCOCn1nc(C(=O)O)c2c1CCCCC2]

Prepared in an analogous manner to 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2), replacing 4-(1-((2-(trimethylsilyl)ethoxy)methyl)-1H-pyrazol-4-yl)cyclohexanone (Example B1) with cycloheptanone (commercial).

### Example C43

### 5,5-difluoro-5a-methyl-1,4,4a,5,5a,6-hexahydrocyclopropa[f]indazole-3-carboxylic acid

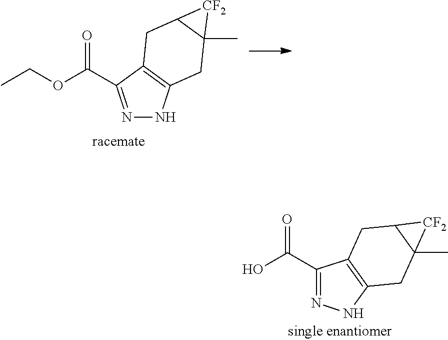 [SMILES: CCOC(=O)c1n[nH]c2c1CC1C(F)(F)C1(C)C2 ; CC12Cc3[nH]nc(C(=O)O)c3CC1C2(F)F]

Racemic ethyl 5,5-difluoro-5a-methyl-1,4,4a,5,5a,6-hexahydrocyclopropa[f]indazole-3-carboxylate (intermediate toward Example C37) was separated by SFC with a chiral stationary phase. The two enantiomers were then carried forward to Examples 138a and 138b. The isomer which provided Example 138b was then hydrolyzed as described above for Example C37, and used as Example C43.

### Example C44

### 6-methyl-6-morpholino-1-((2-(trimethylsilyl)ethoxy)methyl)-4,5,6,7-tetrahydro-1H-indazole-3-carboxylic acid

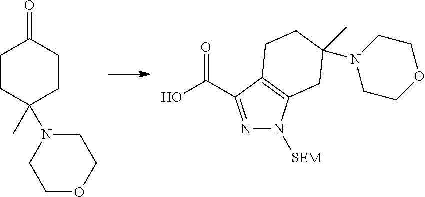 [SMILES: CC1(N2CCOCC2)CCC(=O)CC1 ; CC1(N2CCOCC2)CCc2c(C(=O)O)nn(COCC[Si](C)(C)C)c2C1]

Prepared in an analogous manner to 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1), replacing 4-(1-((2-(trimethylsilyl)ethoxy)methyl)-1H-pyrazol-4-yl)cyclohexanone (Example B1) with 4-methyl-4-morpholinocyclohexanone (Example A16).

### Synthesis of Final Compounds

### Examples 1a and 1b

### N-(1-(3-cyanobenzyl)-1H-pyrazol-4-yl)-6-(1H-pyrazol-4-yl)-4,5,6,7-tetrahydro-1H-indazole-3-carboxamide

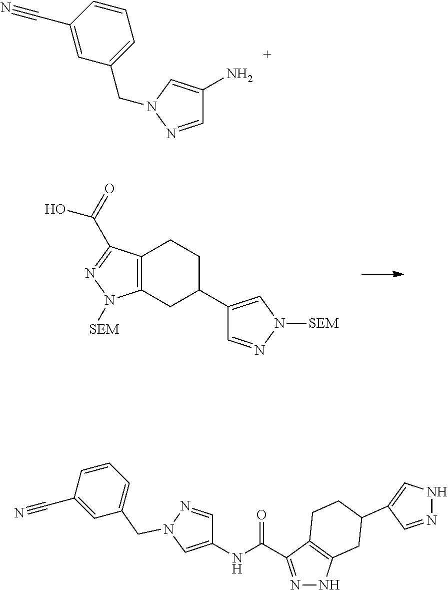 [SMILES: N#Cc1cccc(Cn2cc(N)cn2)c1 ; C[Si](C)(C)CCOCn1cc(C2CCc3c(C(=O)O)nn(COCC[Si](C)(C)C)c3C2)cn1 ; N#Cc1cccc(Cn2cc(NC(=O)c3n[nH]c4c3CCC(c3cn[nH]c3)C4)cn2)c1]

To a solution of 3-((4-amino-1H-pyrazol-1-yl)methyl)benzonitrile (Example A1, 101 mg, 0.507 mmol) and 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1, 250 mg, 0.507 mmol) in DMF (1.8 mL) was added O-(benzotriazol-1-yl)-N,N,N′,′N-tetramethyluronium tetrafluoroborate (TBTU, 235 mg, 0.710 mmol) and diisopropylethyl amine (0.265 mL, 1.52 mmol) and the mixture was stirred overnight at rt. The mixture was diluted with H₂O, extracted with EtOAc (3×), then the combined organic extracts were dried (MgSO₄) and concentrated in vacuo. Purification by CombiFlash (12 g; 100:0 to 0:100 heptane:EtOAc over 14 minutes) provided 260 mg (0.387 mmol; 76%) of the desired amide. This material was diluted with 5 mL 4.0 N HCl in dioxane and heated to 60° C. for 1 hour. After cooling to rt and in vacuo concentration, the residue was diluted with EtOH (12 mL) and NaOH (aq) (5.0 M, 4 mL) and stirred for an additional hour to remove any residual hydroxymethyl acetal. The mixture was diluted with water, extracted with 10% MeOH in CH₂Cl₂ (3×), dried (MgSO₄) and concentrated in vacuo. The residue was first purified by reverse phase HPLC, then the enantiomers were separated by supercritical fluid chromatography (SFC) to provide 18.0 mg of 1a and 13.6 mg of 1b.

SFC conditions: Chiralpak OJ (21.2×250 mm, 5 μm particle size) at 35% methanol w/0.1% NH₄OH; 70 ml/min, 100 bars, 40° C.

1a: ¹H NMR (400 MHz, DMSO-d₆) δ 12.89 (s, 1H), 12.57 (s, 1H), 10.14 (s, 1H), 8.17 (s, 1H), 7.77 (d, J=6.9, 1H), 7.68 (s, 2H), 7.60-7.46 (m, 4H), 5.36 (s, 2H), 3.03-2.82 (m, 3H), 2.73-2.57 (m, 2H), 2.12-1.99 (m, 1H), 1.76-1.61 (m, 1H); MS m/z=413 (M+H); SFC retention time: 1.22 min.

1b: ¹H NMR (400 MHz, DMSO-d₆) δ 12.88 (s, 1H), 12.58 (s, 1H), 10.14 (s, 1H), 8.17 (s, 1H), 7.77 (d, J=6.9, 1H), 7.68 (s, 2H), 7.60-7.46 (m, 4H), 5.36 (s, 2H), 3.03-2.82 (m, 3H), 2.72-2.60 (m, 2H), 2.11-2.01 (m, 1H), 1.76-1.61 (m, 1H); MS m/z=413 (M+H); SFC retention time: 0.96 min.

It should be noted that although this procedure is representative for all the examples that follow, in general yields are significantly higher than that obtained in this case. Also, for cases when final compounds are achiral, purification is accomplished by preparative reverse phase HPLC only (no SFC).

### Examples 2a and 2b

### N-(1-(3-cyanobenzyl)-1H-pyrazol-4-yl)-5-(1H-pyrazol-4-yl)-4,5,6,7-tetrahydro-1H-indazole-3-carboxamide

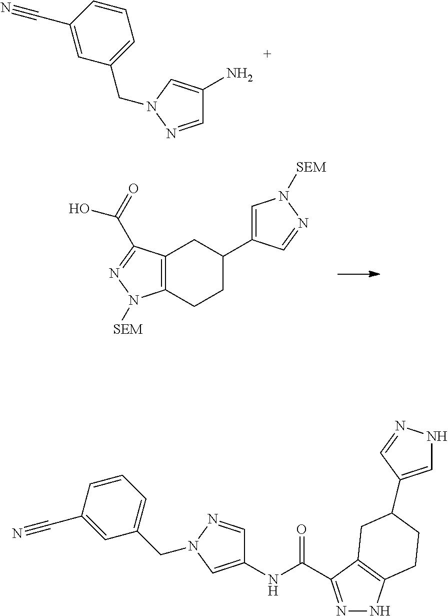 [SMILES: N#Cc1cccc(Cn2cc(N)cn2)c1 ; C[Si](C)(C)CCOCn1cc(C2CCc3c(c(C(=O)O)nn3COCC[Si](C)(C)C)C2)cn1 ; N#Cc1cccc(Cn2cc(NC(=O)c3n[nH]c4c3CC(c3cn[nH]c3)CC4)cn2)c1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-5-(1-((2-(trimethylsilyl)ethoxy)methyl)-1H-pyrazol-4-yl)-4,5,6,7-tetrahydro-1H-indazole-3-carboxylic acid (Example C2).

SFC conditions: Chiralpak AS (21.2×150 mm, 5 μm particle size) at 35% methanol w/0.1% NH₄OH; 70 ml/min, 100 bars, 40° C.

2a: ¹H NMR (400 MHz, DMSO-d₆) δ 13.10-12.30 (m, 2H), 10.16 (s, 1H), 8.16 (s, 1H), 7.77 (d, J=6.9, 1H), 7.68 (s, 2H), 7.60-7.45 (m, 4H), 5.36 (s, 2H), 3.16 (dd, J=16.2, 5.0, 1H), 2.92-2.82 (m, 1H), 2.75-2.68 (m, 2H), 2.58 (dd, J=16.2, 10.1, 1H), 2.14-2.08 (m, 1H), 1.81-1.68 (m, 1H); MS m/z=413 (M+H); SFC retention time: 0.58 min.

2b: ¹H NMR (400 MHz, DMSO-d₆) δ 12.88 (s, 1H), 12.55 (s, 1H), 10.16 (s, 1H), 8.16 (s, 1H), 7.77 (d, J=6.9, 1H), 7.68 (s, 2H), 7.60-7.51 (m, 3H), 7.40 (s, 1H), 5.36 (s, 2H), 3.16 (dd, J=16.2, 5.0, 1H), 2.92-2.82 (m, 1H), 2.76-2.68 (m, 2H), 2.58 (dd, J=16.2, 10.1, 1H), 2.14-2.08 (m, 1H), 1.82-1.68 (m, 1H); MS m/z=413 (M+H); SFC retention time: 0.65 min.

### Example 3

### N-(1-(3-cyanobenzyl)-1H-pyrazol-4-yl)-4,5,6,7-tetrahydro-1H-indazole-3-carboxamide

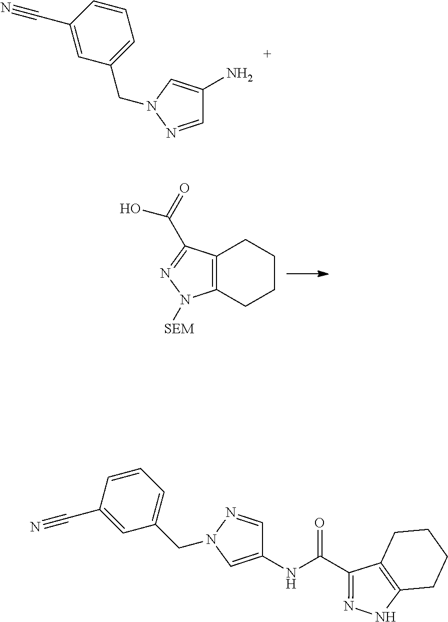 [SMILES: N#Cc1cccc(Cn2cc(N)cn2)c1 ; C[Si](C)(C)CCOCn1nc(C(=O)O)c2c1CCCC2 ; N#Cc1cccc(Cn2cc(NC(=O)c3n[nH]c4c3CCCC4)cn2)c1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-4,5,6,7-tetrahydro-1H-indazole-3-carboxylic acid (Example C3).

¹H NMR (400 MHz, DMSO-d₆) δ 12.82 (s, 1H), 10.08 (d, J=12.0, 1H), 8.16 (s, 1H), 7.77 (d, J=6.8, 1H), 7.67 (s, 2H), 7.60-7.52 (m, 2H), 5.35 (s, 2H), 2.67 (t, J=5.5, 2H), 2.61 (t, J=5.8, 2H), 1.77-1.62 (m, 4H); MS: m/z=347 (M+H).

### Examples 4a and 4b

### N-(1-(3-cyanobenzyl)-1H-pyrazol-4-yl)-6-(pyrimidin-5-yl)-4,5,6,7-tetrahydro-1H-indazole-3-carboxamide

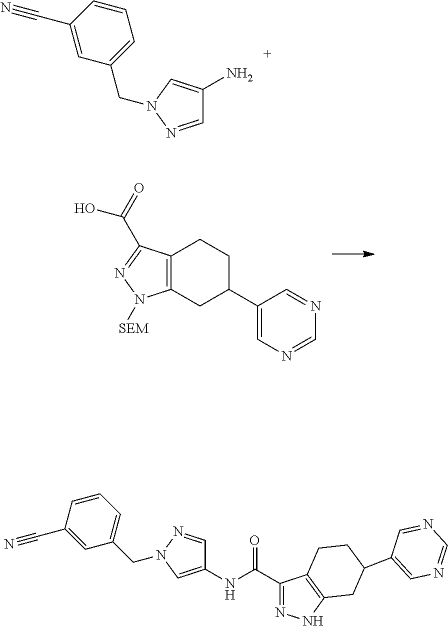 [SMILES: N#Cc1cccc(Cn2cc(N)cn2)c1 ; C[Si](C)(C)CCOCn1nc(C(=O)O)c2c1CC(c1cncnc1)CC2 ; N#Cc1cccc(Cn2cc(NC(=O)c3n[nH]c4c3CCC(c3cncnc3)C4)cn2)c1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6-(pyrimidin-5-yl)-1-((2-(trimethylsilyl)ethoxy)methyl)-4,5,6,7-tetrahydro-1H-indazole-3-carboxylic acid (Example C4).

SFC Conditions: Phenomenex Cellulose-1 (21.2×250 mm, 5 μm particle size) at 50% methanol w/0.1% NH₄OH; 60 ml/min, 100 bars, 40° C.

4a: ¹H NMR (400 MHz, DMSO-d₆) δ 12.99 (s, 1H), 10.21 (s, 1H), 9.08 (s, 1H), 8.82 (s, 2H), 8.18 (s, 1H), 7.79-7.75 (m, 1H), 7.71-7.66 (m, 2H), 7.60-7.52 (m, 2H), 5.36 (s, 2H), 3.13-3.03 (m, 1H), 3.02-2.91 (m, 2H), 2.90-2.80 (m, 1H), 2.76-2.67 (m, 1H), 2.06-1.87 (m, 2H); m/z=425 (M+H); SFC retention time: 1.03 min.

4b: ¹H NMR (400 MHz, DMSO-d₆) δ 12.99 (s, 1H), 10.21 (s, 1H), 9.08 (s, 1H), 8.82 (s, 2H), 8.18 (s, 1H), 7.80-7.75 (m, 1H), 7.70-7.67 (m, 2H), 7.60-7.52 (m, 2H), 5.36 (s, 2H), 3.13-3.03 (m, 1H), 3.02-2.91 (m, 2H), 2.90-2.80 (m, 1H), 2.76-2.68 (m, 1H), 2.06-1.87 (m, 2H); m/z=425 (M+H); SFC retention time: 1.31 min.

### Examples 5a and 5b

### N-(1-(3-cyanobenzyl)-1H-pyrazol-4-yl)-6-hydroxy-4,5,6,7-tetrahydro-1H-indazole-3-carboxamide

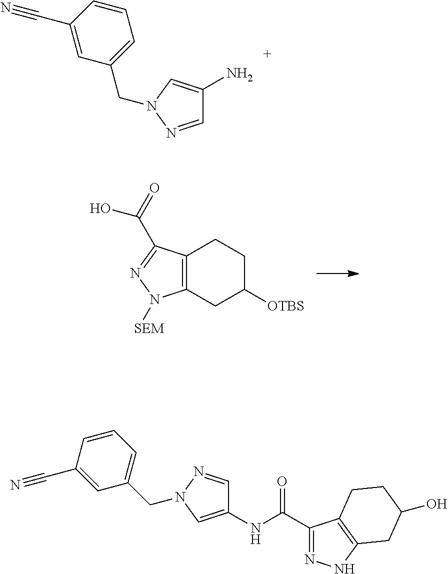 [SMILES: N#Cc1cccc(Cn2cc(N)cn2)c1 ; CC(C)(C)[Si](C)(C)OC1CCc2c(C(=O)O)nn(COCC[Si](C)(C)C)c2C1 ; N#Cc1cccc(Cn2cc(NC(=O)c3n[nH]c4c3CCC(O)C4)cn2)c1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6-(tert-butyldimethylsilyloxy)-1-((2-(trimethylsilyl)ethoxy)methyl)-4,5,6,7-tetrahydro-1H-indazole-3-carboxylic acid (Example C5).

SFC Conditions: Phenomenex Cellulose-4 (21.2×150 mm, 5 μm particle size) at 40% methanol w/0.10% NH₄OH; 70 ml/min, 100 bars, 40° C.

5a: ¹H NMR (400 MHz, DMSO-d₆) δ 12.82 (s, 1H), 10.13 (s, 1H), 8.17 (s, 1H), 7.79-7.75 (m, 1H), 7.68 (d, J=4.0, 2H), 7.59-7.51 (m, 2H), 5.35 (s, 2H), 4.84 (d, J=3.8, 1H), 4.03-3.93 (m, 1H), 2.91-2.74 (m, 2H), 2.69-2.57 (m, 1H), 2.53-2.43 (m, 1H), 1.85-1.75 (m, 1H), 1.71-1.59 (m, 1H); MS: m/z=363 (M+H); SFC retention time: 0.61 min.

5b: ¹H NMR (400 MHz, DMSO-d₆) δ 12.82 (s, 1H), 10.13 (s, 1H), 8.17 (s, 1H), 7.79-7.75 (m, 1H), 7.67 (d, J=4.6, 2H), 7.59-7.52 (m, 2H), 5.35 (s, 2H), 4.84 (d, J=3.8, 1H), 4.03-3.93 (m, 1H), 2.91-2.74 (m, 2H), 2.69-2.57 (m, 1H), 2.53-2.41 (m, 1H), 1.85-1.75 (m, 1H), 1.71-1.59 (m, 1H); MS: m/z=363 (M+H); SFC retention time: 0.82 min.

### Example 6

### N-(1-benzyl-1H-pyrazol-4-yl)-6,6-dimethyl-4,5,6,7-tetrahydro-1H-indazole-3-carboxamide

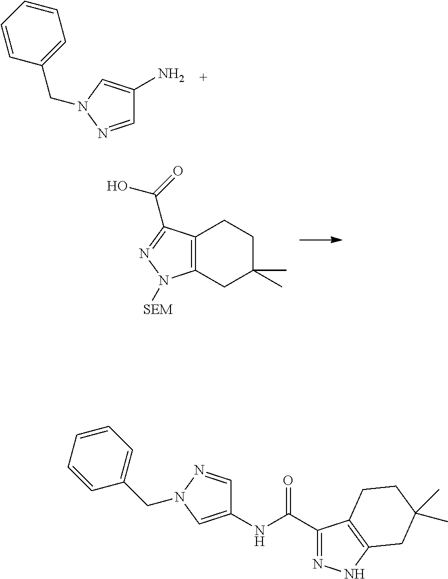 [SMILES: Nc1cnn(Cc2ccccc2)c1 ; CC1(C)CCc2c(C(=O)O)nn(COCC[Si](C)(C)C)c2C1 ; CC1(C)CCc2c(C(=O)Nc3cnn(Cc4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-benzyl-1H-pyrazol-4-amine (Example A2).

¹H NMR (400 MHz, DMSO-d₆) δ 12.77 (s, 1H), 10.04 (s, 1H), 8.07 (s, 1H), 7.64 (s, 1H), 7.37-7.20 (m, 5H), 5.27 (s, 2H), 2.66 (t, J=6.3, 2H), 2.38 (s, 2H), 1.47 (t, J=6.4, 2H), 0.96 (s, 6H); MS: m/z=350 (M+H).

### Example 7

### N-(1-benzyl-1H-pyrazol-4-yl)-5,5-dimethyl-4,5,6,7-tetrahydro-1H-indazole-3-carboxamide

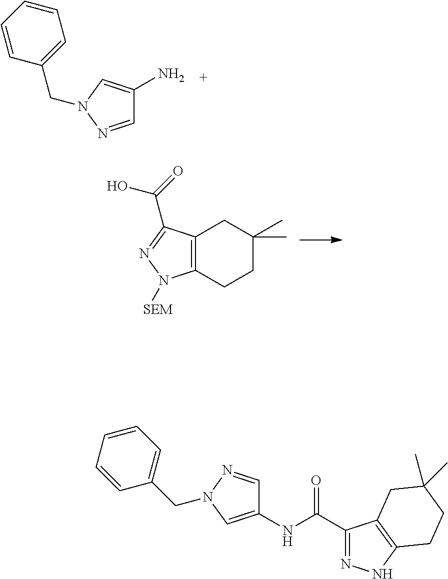 [SMILES: Nc1cnn(Cc2ccccc2)c1 ; CC1(C)CCc2c(c(C(=O)O)nn2COCC[Si](C)(C)C)C1 ; CC1(C)CCc2[nH]nc(C(=O)Nc3cnn(Cc4ccccc4)c3)c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5,5-dimethyl-1-((2-(trimethylsilyl)ethoxy)methyl)-4,5,6,7-tetrahydro-1H-indazole-3-carboxylic acid (Example C7) and 3-((4-amino-1H-pyrazol-1-yl)methyl)benzonitrile (Example A1) with 1-benzyl-1H-pyrazol-4-amine (Example A2).

¹H NMR (400 MHz, DMSO-d₆) δ 12.84 (s, 1H), 10.07 (s, 1H), 8.05 (s, 1H), 7.64 (s, 1H), 7.37-7.26 (m, 3H), 7.25-7.20 (m, 2H), 5.27 (s, 2H), 2.60 (t, J=6.4, 2H), 2.48 (s, 2H), 1.51 (t, J=6.4, 2H), 0.94 (s, 6H); MS: m/z=350 (M+H).

### Examples 8a and 8b

### N-(1-benzyl-1H-pyrazol-4-yl)-5-methyl-4,5,6,7-tetrahydro-1H-indazole-3-carboxamide

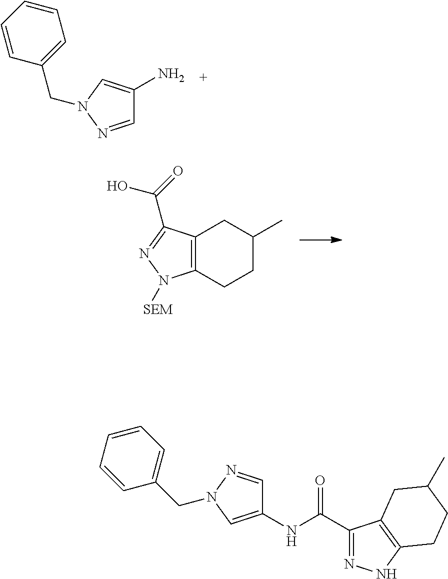 [SMILES: Nc1cnn(Cc2ccccc2)c1 ; CC1CCc2c(c(C(=O)O)nn2COCC[Si](C)(C)C)C1 ; CC1CCc2[nH]nc(C(=O)Nc3cnn(Cc4ccccc4)c3)c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5-methyl-1-((2-(trimethylsilyl)ethoxy)methyl)-4,5,6,7-tetrahydro-1H-indazole-3-carboxylic acid (Example C8) and 3-((4-amino-1H-pyrazol-1-yl)methyl)benzonitrile (Example A1) with 1-benzyl-1H-pyrazol-4-amine (Example A2).

SFC Conditions: ChiralPak IA (21.2×250 mm, 5 μm particle size) at 40% methanol w/0.1% NH₄OH; 50 ml/min, 100 bars, 40° C.

8a: ¹H NMR (400 MHz, DMSO-d₆) δ 12.81 (s, 1H), 10.07 (s, 1H), 8.06 (s, 1H), 7.64 (s, 1H), 7.37-7.20 (m, 5H), 5.27 (s, 2H), 2.90 (dd, J=16.3, 5.0, 1H), 2.73-2.53 (m, 2H), 2.15 (dd, J=16.3, 9.7, 1H), 1.88-1.67 (m, 2H), 1.42-1.30 (m, 1H), 1.03 (d, J=6.6, 3H); MS: m/z=336 (M+H); SFC retention time: 0.62 min.

8b: ¹H NMR (400 MHz, DMSO-d₆) δ 12.81 (s, 1H), 10.07 (s, 1H), 8.06 (s, 1H), 7.64 (s, 1H), 7.38-7.20 (m, 5H), 5.27 (s, 2H), 2.90 (dd, J=16.2, 4.9, 1H), 2.73-2.53 (m, 2H), 2.15 (dd, J=16.3, 9.7, 1H), 1.88-1.66 (m, 2H), 1.44-1.30 (m, 1H), 1.03 (d, J=6.6, 3H); MS: m/z=336 (M+H); SFC retention time: 0.74 min.

### Examples 9a and 9b

### N-(1-benzyl-1H-pyrazol-4-yl)-6-methyl-4,5,6,7-tetrahydro-1H-indazole-3-carboxamide

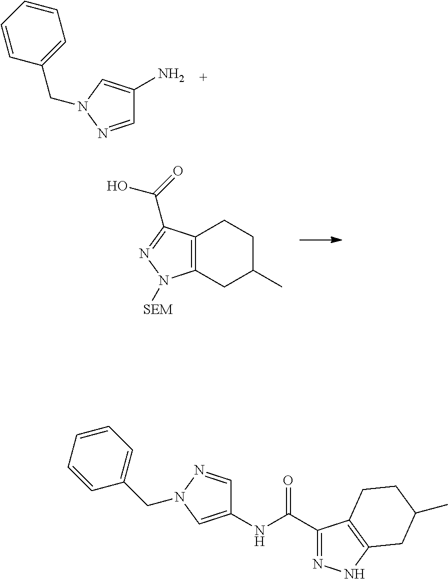 [SMILES: Nc1cnn(Cc2ccccc2)c1 ; CC1CCc2c(C(=O)O)nn(COCC[Si](C)(C)C)c2C1 ; CC1CCc2c(C(=O)Nc3cnn(Cc4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6-methyl-1-((2-(trimethylsilyl)ethoxy)methyl)-4,5,6,7-tetrahydro-1H-indazole-3-carboxylic acid (Example C9) and 3-((4-amino-1H-pyrazol-1-yl)methyl)benzonitrile (Example A1) with 1-benzyl-1H-pyrazol-4-amine (Example A2).

SFC Conditions: Phenomenex Cellulose-4 (21.2×150 mm, 5 μm particle size) at 40% methanol w/0.1% NH₄OH; 70 ml/min, 100 bars, 40° C.

9a: ¹H NMR (400 MHz, DMSO-d₆) δ 12.79 (s, 1H), 10.07 (s, 1H), 8.07 (s, 1H), 7.64 (s, 1H), 7.37-7.25 (m, 3H), 7.25-7.20 (m, 2H), 5.27 (s, 2H), 2.87-2.77 (m, 1H), 2.73 (dd, J=15.8, 5.2, 1H), 2.61-2.48 (m, 1H), 2.18 (dd, J=15.9, 9.6, 1H), 1.89-1.73 (m, 2H), 1.38-1.24 (m, 1H), 1.04 (d, J=6.6, 3H); MS: m/z=336 (M+H); SFC retention time: 0.57 min.

9b: ¹H NMR (400 MHz, DMSO-d₆) δ 10.07 (s, 1H), 8.36 (s, 1H), 8.07 (s, 1H), 7.64 (s, 1H), 7.37-7.25 (m, 3H), 7.25-7.20 (m, 2H), 5.27 (s, 2H), 2.87-2.78 (m, 1H), 2.73 (dd, J=15.8, 5.2, 1H), 2.60-2.48 (m, 1H), 2.18 (dd, J=15.9, 9.6, 1H), 1.90-1.73 (m, 2H), 1.38-1.25 (m, 1H), 1.04 (d, J=6.6, 3H); MS: m/z=336 (M+H); SFC retention time: 0.49 min.

### Examples 10a and 10b

### N-(1-(3-(dimethylamino)-1-phenylpropyl)-1H-pyrazol-4-yl)-6,6-dimethyl-4,5,6,7-tetrahydro-1H-indazole-3-carboxamide

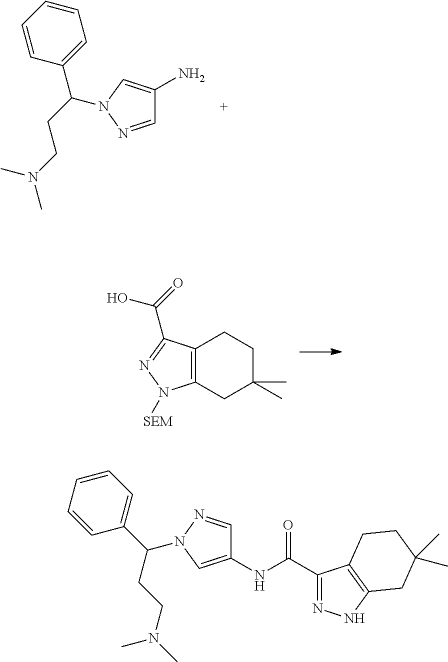 [SMILES: CN(C)CCC(c1ccccc1)n1cc(N)cn1 ; CC1(C)CCc2c(C(=O)O)nn(COCC[Si](C)(C)C)c2C1 ; CN(C)CCC(c1ccccc1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(3-(dimethylamino)-1-phenylpropyl)-1H-pyrazol-4-amine (Example A3).

SFC Conditions: Phenomenex Cellulose-4 (21.2×150 mm, 5 μm particle size) at 45% methanol w/0.10% NH₄OH; 70 ml/min, 100 bars, 40° C.

10a: ¹H NMR (400 MHz, DMSO-d₆) δ 12.78 (s, 1H), 10.05 (s, 1H), 8.08 (s, 1H), 7.66 (s, 1H), 7.37-7.22 (m, 5H), 5.42 (dd, J=9.3, 5.3, 1H), 2.66 (t, J=6.0, 2H), 2.38 (s, 2H), 2.25-2.02 (m, 10H), 1.47 (t, J=6.4, 2H), 0.96 (s, 6H); MS: m/z=421 (M+H); SFC retention time: 0.37 min.

10b: ¹H NMR (400 MHz, DMSO-d₆) δ 12.78 (s, 1H), 10.04 (s, 1H), 8.08 (s, 1H), 7.66 (s, 1H), 7.36-7.23 (m, 5H), 5.42 (dd, J=9.2, 5.6, 1H), 2.66 (t, J=6.2, 2H), 2.38 (s, 2H), 2.23-2.00 (m, 10H), 1.47 (t, J=6.3, 2H), 0.96 (s, 6H); MS: m/z=421 (M+H); SFC retention time: 0.59 min.

### Example 11

### N-(1-benzyl-1H-pyrazol-4-yl)-1′,4′,5′,7′-tetrahydrospiro[cyclopropane-1,6′-indazole]-3-carboxamide

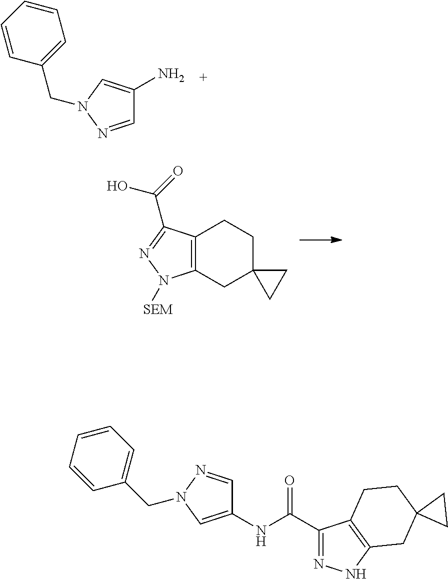 [SMILES: Nc1cnn(Cc2ccccc2)c1 ; C[Si](C)(C)CCOCn1nc(C(=O)O)c2c1CC1(CC2)CC1 ; O=C(Nc1cnn(Cc2ccccc2)c1)c1n[nH]c2c1CCC1(CC1)C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1′-((2-(trimethylsilyl)ethoxy)methyl)-1′,4′,5′,7′-tetrahydrospiro[cyclopropane-1,6′-indazole]-3′-carboxylic acid (Example C10) and 3-((4-amino-1H-pyrazol-1-yl)methyl)benzonitrile (Example A1) with 1-benzyl-1H-pyrazol-4-amine (Example A2).

¹H NMR (400 MHz, DMSO-d₆) δ 12.89 (s, 1H), 10.13 (s, 1H), 8.07 (s, 1H), 7.65 (s, 1H), 7.38-7.26 (m, 3H), 7.26-7.21 (m, 2H), 5.28 (s, 2H), 4.38 (d, J=5.7, 2H), 4.27 (d, J=5.7, 2H), 2.99 (s, 2H), 2.69 (t, J=6.3, 2H), 2.03 (t, J=6.3, 2H); MS: m/z=348 (M+H).

### Example 12

### N-(1-benzyl-1H-pyrazol-4-yl)-1,4,5,7-tetrahydrospiro[indazole-6,3′-oxetane]-3-carboxamide

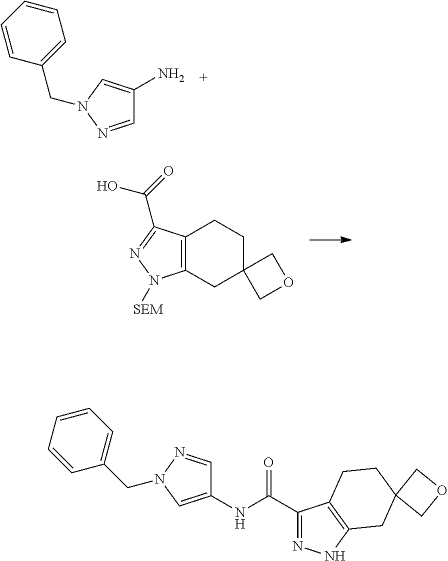 [SMILES: Nc1cnn(Cc2ccccc2)c1 ; C[Si](C)(C)CCOCn1nc(C(=O)O)c2c1CC1(CC2)COC1 ; O=C(Nc1cnn(Cc2ccccc2)c1)c1n[nH]c2c1CCC1(COC1)C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1,4,5,7-tetrahydrospiro[indazole-6,3′-oxetane]-3-carboxylic acid (Example C11) and 3-((4-amino-1H-pyrazol-1-yl)methyl)benzonitrile (Example A1) with 1-benzyl-1H-pyrazol-4-amine (Example A2).

¹H NMR (400 MHz, DMSO-d₆) δ 12.93 (s, 1H), 10.10 (s, 1H), 8.07 (s, 1H), 7.64 (s, 1H), 7.37-7.25 (m, 3H), 7.25-7.20 (m, 2H), 5.27 (s, 2H), 4.39 (d, J=5.8, 2H), 4.32 (d, J=5.8, 2H), 2.94 (s, 2H), 2.72 (t, J=6.1, 2H), 1.97 (t, J=6.3, 2H); MS: m/z=364 (M+H).

### Example 13

### N-(1-benzyl-1H-pyrazol-4-yl)-1,4,6,7-tetrahydrospiro[indazole-5,3′-oxetane]-3-carboxamide

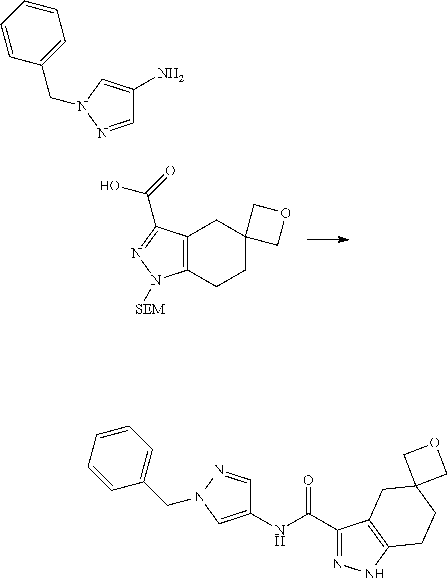 [SMILES: Nc1cnn(Cc2ccccc2)c1 ; C[Si](C)(C)CCOCn1nc(C(=O)O)c2c1CCC1(COC1)C2 ; O=C(Nc1cnn(Cc2ccccc2)c1)c1n[nH]c2c1CC1(CC2)COC1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1,4,6,7-tetrahydrospiro[indazole-5,3′-oxetane]-3-carboxylic acid (Example C12) and 3-((4-amino-1H-pyrazol-1-yl)methyl)benzonitrile (Example A1) with 1-benzyl-1H-pyrazol-4-amine (Example A2).

¹H NMR (400 MHz, DMSO-d₆) δ 12.89 (s, 1H), 10.13 (s, 1H), 8.07 (s, 1H), 7.65 (s, 1H), 7.38-7.26 (m, 3H), 7.26-7.21 (m, 2H), 5.28 (s, 2H), 4.38 (d, J=5.7, 2H), 4.27 (d, J=5.7, 2H), 2.99 (s, 2H), 2.69 (t, J=6.3, 2H), 2.03 (t, J=6.3, 2H); MS: m/z=364 (M+H).

### Example 14a and 14b

### N-(1-benzyl-1H-pyrazol-4-yl)-6-(hydroxymethyl)-4,5,6,7-tetrahydro-1H-indazole-3-carboxamide

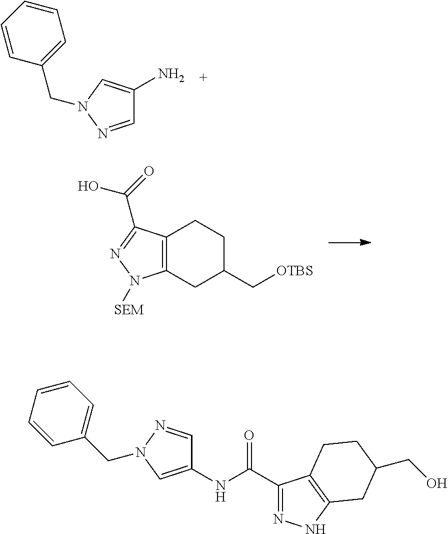 [SMILES: Nc1cnn(Cc2ccccc2)c1 ; CC(C)(C)[Si](C)(C)OCC1CCc2c(C(=O)O)nn(COCC[Si](C)(C)C)c2C1 ; O=C(Nc1cnn(Cc2ccccc2)c1)c1n[nH]c2c1CCC(CO)C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6-((tert-butyldimethylsilyloxy)methyl)-1-((2-(trimethylsilyl)ethoxy)methyl)-4,5,6,7-tetrahydro-1H-indazole-3-carboxylic acid (Example C13) and 3-((4-amino-1H-pyrazol-1-yl)methyl)benzonitrile (Example A1) with 1-benzyl-1H-pyrazol-4-amine (Example A2).

SFC Conditions: Phenomenex Cellulose-2 (21.2×250 mm, 5 μm particle size) at 40% methanol w/0.10% NH₄OH; 70 ml/min, 100 bars, 40° C.

14a: ¹H NMR (400 MHz, DMSO-d₆) δ 12.81 (s, 1H), 10.08 (s, 1H), 8.07 (s, 1H), 7.64 (s, 1H), 7.37-7.26 (m, 3H), 7.26-7.21 (m, 2H), 5.27 (s, 2H), 4.58 (t, J=5.2, 1H), 3.45-3.35 (m, 2H), 2.88-2.78 (m, 1H), 2.72 (dd, J=16.2, 5.0, 1H), 2.58-2.45 (m, 1H), 2.27 (dd, J=16.0, 9.8, 1H), 1.91-1.76 (m, 2H), 1.38-1.25 (m, 1H); MS: m/z=352 (M+H); SFC retention time: 1.11 min.

14b: ¹H NMR (400 MHz, DMSO-d₆) δ 12.61 (s, 1H), 10.08 (s, 1H), 8.07 (s, 1H), 7.64 (s, 1H), 7.37-7.25 (m, 3H), 7.25-7.20 (m, 2H), 5.27 (s, 2H), 4.74-4.38 (m, 1H), 3.44-3.22 (m, 2H), 2.88-2.78 (m, 1H), 2.72 (dd, J=16.1, 5.1, 1H), 2.58-2.46 (m, 1H), 2.27 (dd, J=16.0, 9.7, 1H), 1.91-1.75 (m, 2H), 1.38-1.25 (m, 1H); MS: m/z=352 (M+H); SFC retention time: 0.84 min.

### Examples 15a and 15b

### N-(1-(3-(dimethylamino)-1-phenylpropyl)-1H-pyrazol-4-yl)-1,4,5,7-tetrahydrospiro[indazole-6,3′-oxetane]-3-carboxamide

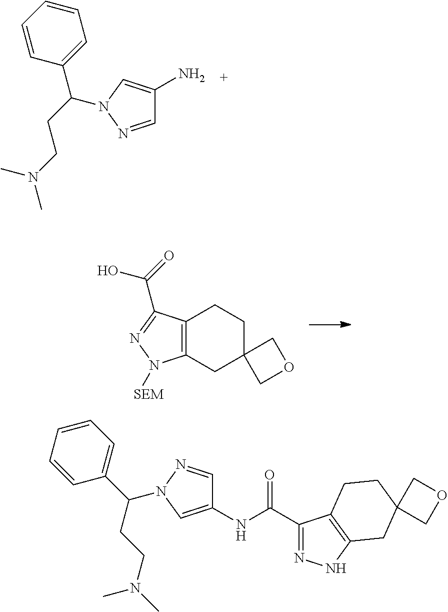 [SMILES: CN(C)CCC(c1ccccc1)n1cc(N)cn1 ; C[Si](C)(C)CCOCn1nc(C(=O)O)c2c1CC1(CC2)COC1 ; CN(C)CCC(c1ccccc1)n1cc(NC(=O)c2n[nH]c3c2CCC2(COC2)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1,4,5,7-tetrahydrospiro[indazole-6,3′-oxetane]-3-carboxylic acid (Example C11) and 3-((4-amino-1H-pyrazol-1-yl)methyl)benzonitrile (Example A1) with 1-(3-(dimethylamino)-1-phenylpropyl)-1H-pyrazol-4-amine (Example A3).

SFC Conditions: Phenomenex Cellulose-2 (21.2×250 mm, 5 μm particle size) at 50% methanol w/0.10% NH₄OH; 50 ml/min, 100 bars, 40° C.

15a: ¹H NMR (400 MHz, DMSO-d₆) δ 12.92 (s, 1H), 10.07 (s, 1H), 8.07 (s, 1H), 7.65 (s, 1H), 7.37-7.22 (m, 5H), 5.42 (dd, J=9.0, 5.7, 1H), 4.38 (d, J=5.8, 2H), 4.32 (d, J=5.8, 2H), 2.93 (s, 2H), 2.72 (t, J=6.1, 2H), 2.54-2.41 (m, 1H), 2.23-2.01 (m, 9H), 1.97 (t, J=6.3, 2H); MS: m/z=435 (M+H); SFC retention time: 0.62 min.

15b: ¹H NMR (400 MHz, DMSO-d₆) δ 12.92 (s, 1H), 10.07 (s, 1H), 8.07 (s, 1H), 7.65 (s, 1H), 7.36-7.22 (m, 5H), 5.42 (dd, J=9.0, 5.6, 1H), 4.38 (d, J=5.8, 2H), 4.31 (d, J=5.8, 2H), 2.93 (s, 2H), 2.72 (t, J=6.1, 2H), 2.53-2.39 (m, 1H), 2.23-2.01 (m, 9H), 1.97 (t, J=6.3, 2H); MS: m/z=435 (M+H); SFC retention time: 1.26 min.

### Examples 16a and 16b

### N-(1-(3-(dimethylamino)-1-phenylpropyl)-1H-pyrazol-4-yl)-1′,4′,5′,7′-tetrahydrospiro[cyclo propane-1,6′-indazole]-3′-carboxamide

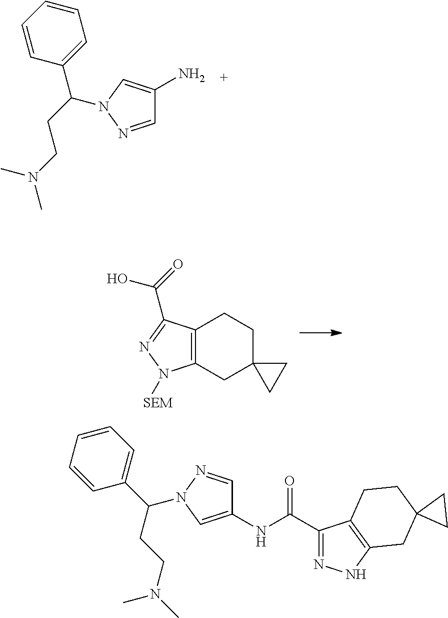

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1′,4′,5′,7′-tetrahydrospiro[cyclopropane-1,6′-indazole]-3′-carboxylic acid (Example C10) and 3-((4-amino-1H-pyrazol-1-yl)methyl)benzonitrile (Example A1) with 1-(3-(dimethylamino)-1-phenylpropyl)-1H-pyrazol-4-amine (Example A3).

SFC Conditions: Phenomenex Cellulose-4 (21.2×150 mm, 5 μm particle size) at 50% methanol w/0.1% NH₄OH; 50 ml/min, 100 bars, 40° C.

16a: ¹H NMR (400 MHz, DMSO-d₆) δ 12.82 (s, 1H), 10.07 (s, 1H), 8.08 (s, 1H), 7.67 (s, 1H), 7.38-7.22 (m, 5H), 5.42 (dd, J=9.0, 5.5, 1H), 2.71 (t, J=5.9, 2H), 2.54-2.40 (m, 3H), 2.23-1.98 (m, 9H), 1.48 (t, J=6.0, 2H), 0.44-0.36 (m, 4H); MS: m/z=419 (M+H); SFC retention time: 0.38 min.

16b: ¹H NMR (400 MHz, DMSO-d₆) δ 12.48 (s, 1H), 10.09 (s, 1H), 8.08 (s, 1H), 7.66 (s, 1H), 7.38-7.23 (m, 5H), 5.42 (dd, J=9.0, 5.7, 1H), 2.71 (t, J=6.0, 2H), 2.54-2.40 (m, 3H), 2.24-1.98 (m, 9H), 1.48 (t, J=6.1, 2H), 0.44-0.35 (m, 4H); MS: m/z=419 (M+H); SFC retention time: 0.67 min.

### Example 17

### N-(1-benzyl-1H-pyrazol-4-yl)-5,5-difluoro-4,5,6,7-tetrahydro-1H-indazole-3-carboxamide

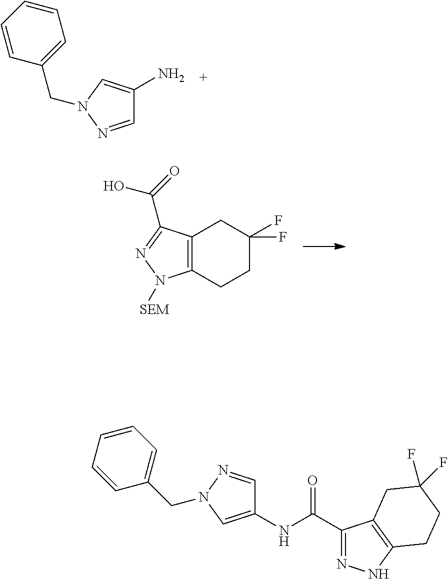 [SMILES: Nc1cnn(Cc2ccccc2)c1 ; C[Si](C)(C)CCOCn1nc(C(=O)O)c2c1CCC(F)(F)C2 ; O=C(Nc1cnn(Cc2ccccc2)c1)c1n[nH]c2c1CC(F)(F)CC2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5,5-difluoro-1-((2-(trimethylsilyl)ethoxy)methyl)-4,5,6,7-tetrahydro-1H-indazole-3-carboxylic acid (Example C14) and 3-((4-amino-1H-pyrazol-1-yl)methyl)benzonitrile (Example A1) with 1-benzyl-1H-pyrazol-4-amine (Example A2).

¹H NMR (400 MHz, DMSO-d₆) δ 13.13 (s, 1H), 10.24 (s, 1H), 8.09 (s, 1H), 7.64 (s, 1H), 7.37-7.26 (m, 3H), 7.25-7.21 (m, 2H), 5.28 (s, 2H), 3.22 (t, J=14.4, 2H), 2.84 (t, J=6.6, 2H), 2.35-2.21 (m, 2H); MS: m/z=358 (M+H).

### Example 18

### N-(1-benzyl-1H-pyrazol-4-yl)-6,6-difluoro-4,5,6,7-tetrahydro-1H-indazole-3-carboxamide

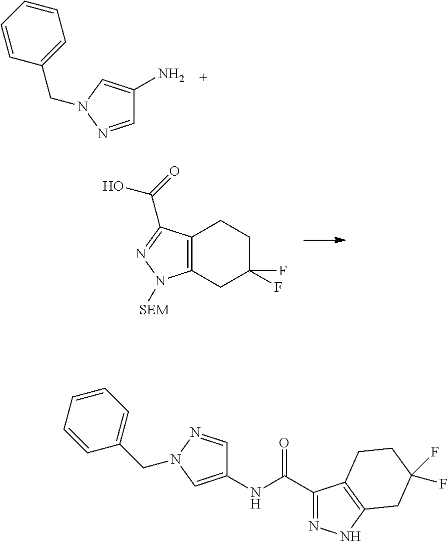 [SMILES: Nc1cnn(Cc2ccccc2)c1 ; C[Si](C)(C)CCOCn1nc(C(=O)O)c2c1CC(F)(F)CC2 ; O=C(Nc1cnn(Cc2ccccc2)c1)c1n[nH]c2c1CCC(F)(F)C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-difluoro-1-((2-(trimethylsilyl)ethoxy)methyl)-4,5,6,7-tetrahydro-1H-indazole-3-carboxylic acid (Example C15) and 3-((4-amino-1H-pyrazol-1-yl)methyl)benzonitrile (Example A1) with 1-benzyl-1H-pyrazol-4-amine (Example A2).

¹H NMR (400 MHz, DMSO-d₆) δ 13.12 (s, 1H), 10.22 (s, 1H), 8.08 (s, 1H), 7.64 (s, 1H), 7.37-7.26 (m, 3H), 7.26-7.21 (m, 2H), 5.28 (s, 2H), 3.35-3.21 (m, 2H), 2.87 (t, J=6.4, 2H), 2.28-2.14 (m, 2H); MS: m/z=358 (M+H).

### Example 19a and 19b

### N-(1-benzyl-1H-pyrazol-4-yl)-6-(hydroxymethyl)-6-methyl-4,5,6,7-tetrahydro-1H-indazole-3-carboxamide

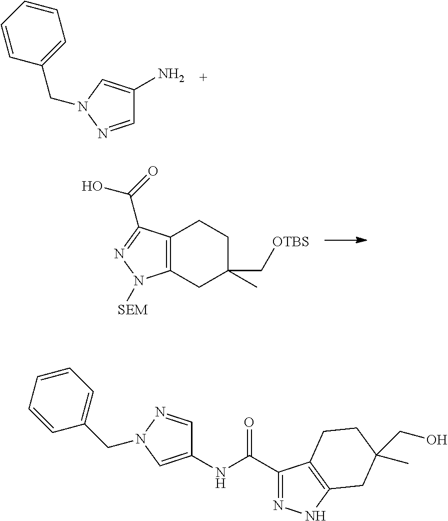 [SMILES: Nc1cnn(Cc2ccccc2)c1 ; CC1(CO[Si](C)(C)C(C)(C)C)CCc2c(C(=O)O)nn(COCC[Si](C)(C)C)c2C1 ; CC1(CO)CCc2c(C(=O)Nc3cnn(Cc4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6-((tert-butyldimethylsilyloxy)methyl)-6-methyl-1-((2-(trimethylsilyl)ethoxy)methyl)-4,5,6,7-tetrahydro-1H-indazole-3-carboxylic acid (Example C16) and 3-((4-amino-1H-pyrazol-1-yl)methyl)benzonitrile (Example A1) with 1-benzyl-1H-pyrazol-4-amine (Example A2).

SFC Conditions: Phenomenex Amylose-2 (21.2×250 mm, 5 μm particle size) at 35% methanol w/0.10% NH₄OH; 40 ml/min, 100 bars, 40° C.

19a: ¹H NMR (400 MHz, DMSO-d₆) δ 12.79 (s, 1H), 10.08 (s, 1H), 8.07 (s, 1H), 7.64 (s, 1H), 7.37-7.26 (m, 3H), 7.25-7.20 (m, 2H), 5.27 (s, 2H), 4.62 (t, J=5.4, 1H), 3.21 (d, J=5.4, 2H), 2.72 (dt, J=16.7, 5.6, 1H), 2.63-2.48 (m, 2H), 2.26 (d, J=16.1, 1H), 1.59-1.49 (m, 1H), 1.47-1.37 (m, 1H), 0.87 (s, 3H); MS: m/z=366 (M+H); SFC retention time: 0.49 min.

19b: ¹H NMR (400 MHz, DMSO-d₆) δ 12.61 (s, 1H), 10.05 (s, 1H), 8.07 (s, 1H), 7.64 (s, 1H), 7.37-7.25 (m, 3H), 7.25-7.20 (m, 2H), 5.27 (s, 2H), 4.63 (s, 1H), 3.21 (s, 2H), 2.78-2.65 (m, 1H), 2.63-2.48 (m, 2H), 2.26 (d, J=16.0, 1H), 1.59-1.49 (m, 1H), 1.47-1.37 (m, 1H), 0.87 (s, 3H); MS: m/z=366 (M+H); SFC retention time: 0.61 min.

### Example 20

### N-(1-(3-cyanobenzyl)-1H-pyrazol-4-42-tetrahydrocyclopenta[c]pyrazole-3-carboxamide

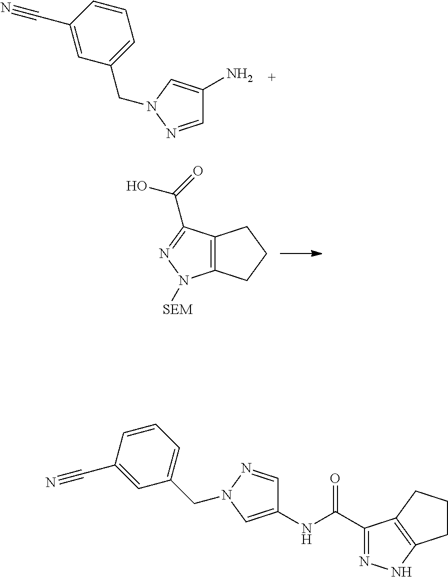

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1,4,5,6-tetrahydrocyclopenta[c]pyrazole-3-carboxylic acid (Example C17).

¹H NMR (400 MHz, DMSO-d₆) δ 12.84 (s, 1H), 10.11 (s, 1H), 8.14 (s, 1H), 7.77 (d, J=6.8, 1H), 7.68 (s, 2H), 7.60-7.51 (m, 2H), 5.36 (s, 2H), 2.68 (s, 4H), 2.56-2.35 (m, 2H); MS: m/z=333 (M+H).

### Example 21

### N-(1-benzyl-1H-pyrazol-4-yl)-5,5-dimethyl-1,4,5,6-tetrahydrocyclopenta[c]pyrazole-3-carboxamide

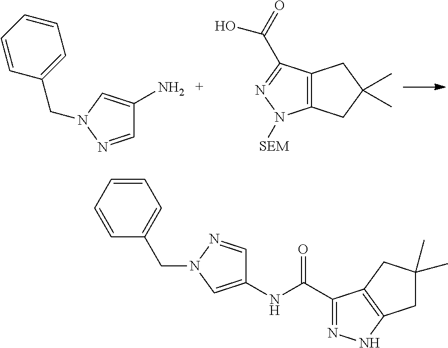

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5,5-dimethyl-1-((2-(trimethylsilyl)ethoxy)methyl)-1,4,5,6-tetrahydrocyclopenta[c]pyrazole-3-carboxylic acid (Example C18) and 3-((4-amino-1H-pyrazol-1-yl)methyl)benzonitrile (Example A1) with 1-benzyl-1H-pyrazol-4-amine (Example A2).

¹H NMR (500 MHz, DMSO-d₆) δ 12.73 (s, 1H), 9.89 (s, 1H), 8.02 (s, 1H), 7.63 (s, 1H), 7.36-7.31 (m, 2H), 7.31-7.26 (m, 1H), 7.25-7.21 (m, 2H), 5.27 (s, 2H), 2.58-2.50 (m, 4H), 1.19 (s, 6H); MS: m/z=336.

### Examples 22a and 22b

### N-(1-benzyl-1H-pyrazol-4-yl)-1,4,4a,5,5a,6-hexahydrocyclopropa[f]indazole-3-carboxamide

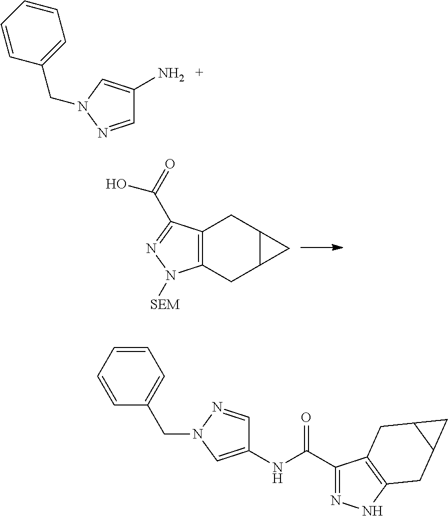

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1,4,4a,5,5a,6-hexahydrocyclopropa[f]indazole-3-carboxylic acid (Example C19a) and 3-((4-amino-1H-pyrazol-1-yl)methyl)benzonitrile (Example A1) with 1-benzyl-1H-pyrazol-4-amine (Example A2). Also, the enantiomers were separated by preparative chiral HPLC instead of SFC.

Chiral HPLC conditions: ChiralPak IC (4.6×250 mm, 5 μm particle size); eluent=hexane (0.1% Et₃N):EtOH 80:20; 1.0 ml/min, 5.2 MPA, 25° C.

22a: ¹H NMR (400 MHz, DMSO-d₆) δ 12.84 (1H, s), 10.11 (1H, s), 8.09 (1H, s), 7.66 (1H, s), 7.39-7.23 (5H, m), 5.29 (2H, s), 2.70-2.65 (1H, t), 2.36-2.22 (2H, m), 2.13-2.08 (1H, t), 1.73-1.67 (1H, m), 0.80-0.91 (1H, m), 0.40-0.50 (1H, m); MS: m/z=334; HPLC retention time: 14.26 min.

22b: ¹H NMR (400 MHz, DMSO-d₆) δ 12.84 (1H, s), 10.11 (1H, s), 8.09 (1H, s), 7.66 (1H, s), 7.39-7.23 (5H, m), 5.29 (2H, s), 2.70-2.65 (1H, t), 2.36-2.22 (2H, m), 2.13-2.08 (1H, t), 1.73-1.67 (1H, m), 0.80-0.91 (1H, m), 0.40-0.50 (1H, m); MS: m/z=334; HPLC retention time: 16.23 min.

### Examples 23a and 23b

### N-(1-benzyl-1H-pyrazol-4-yl)-3,4,5,5a,6,6a-hexahydrocyclopropa[e]indazole-1-carboxamide

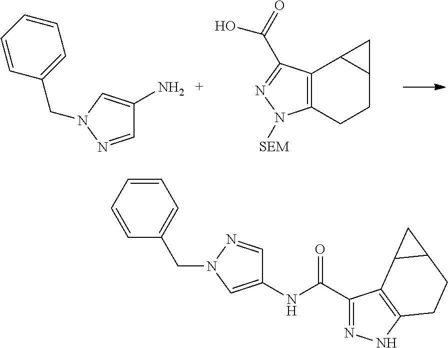

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 3-((2-(trimethylsilyl)ethoxy)methyl)-3,4,5,5a,6,6a-hexahydrocyclopropa[e]indazole-1-carboxylic acid (Example C19b) and 3-((4-amino-1H-pyrazol-1-yl)methyl)benzonitrile (Example A1) with 1-benzyl-1H-pyrazol-4-amine (Example A2). Also, the enantiomers were separated by preparative chiral HPLC instead of SFC.

Chiral HPLC conditions: ChiralPak IB (4.6×250 mm, 5 μm particle size); eluent=hexane (0.1% Et₃N):EtOH 90:10; 1.0 ml/min, 5.2 MPA, 25° C.

23a: ¹H NMR (400 MHz, DMSO-d₆) δ 12.87 (1H, s), 10.11 (1H, s), 8.08 (1H, s), 7.64 (1H, s), 7.36-7.22 (5H, m), 5.28 (2H, s), 3.33-3.23 (1H, t), 3.00-2.82 (3H, m), 1.25-1.19 (2H, t), 0.57-0.55 (1H, m), −0.03-0.05 (1H, d); MS: m/z=334; HPLC retention time: 17.13 min.

23b: ¹H NMR (400 MHz, DMSO-d₆) δ 12.87 (1H, s), 10.11 (1H, s), 8.08 (1H, s), 7.64 (1H, s), 7.36-7.22 (5H, m), 5.28 (2H, s), 3.33-3.23 (1H, t), 3.00-2.82 (3H, m), 1.25-1.19 (2H, t), 0.57-0.55 (1H, m), −0.03-−0.05 (1H, d); MS: m/z=334; HPLC retention time: 18.92 min.

### Examples 24a and 24b

### N-(1-((S)-1-phenylpropyl)-1H-pyrazol-4-yl)-1,4,4a,5,5a,6-hexahydrocyclopropa[f]indazole-3-carboxamide

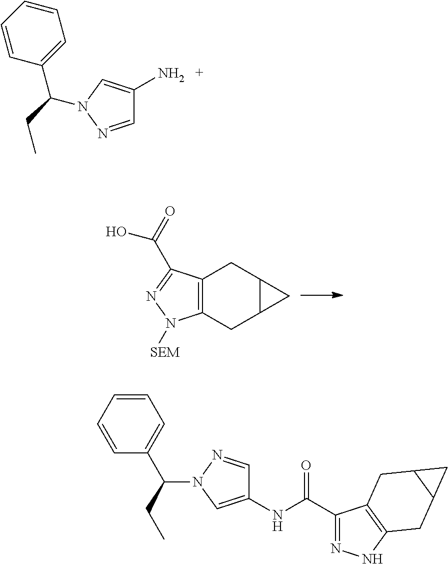

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1,4,4a,5,5a,6-hexahydrocyclopropa[f]indazole-3-carboxylic acid (Example C19a) and 3-((4-amino-1H-pyrazol-1-yl)methyl)benzonitrile (Example A1) with (S)-1-(1-phenylpropyl)-1H-pyrazol-4-amine (Example A4). Also, the diastereomers were separated by preparative chiral HPLC instead of SFC.

Chiral HPLC conditions: ChiralPak IA (4.6×250 mm, 5 μm particle size); eluent=hexane (0.1% Et₃N):EtOH 70:30; 1.0 ml/min, 5.2 MPA, 25° C.

24a: ¹H NMR (400 MHz, CDCl₃) δ 8.62 (s, 1H), 8.17 (s, 1H), 7.58 (S, 1H), 7.36-7.26 (m, 5H), 5.19-5.15 (m, 1H), 2.79-2.74 (m, 1H), 2.52-2.43 (m, 2H), 2.37-2.18 (m, 3H), 1.89 (s, 1H), 1.53-1.52 (m, 1H), 1.09-1.04 (m, 1H), 0.98-0.94 (m, 3H), 0.54-0.50 (m, 1H); MS: m/z=362; HPLC retention time: 9.82 min.

24b: ¹H NMR (400 MHz, CDCl₃) δ 8.62 (s, 1H), 8.17 (s, 1H), 7.58 (S, 1H), 7.36-7.26 (m, 5H), 5.19-5.15 (m, 1H), 2.79-2.74 (m, 1H), 2.52-2.43 (m, 2H), 2.37-2.18 (m, 3H), 1.89 (s, 1H), 1.53-1.52 (m, 1H), 1.09-1.04 (m, 1H), 0.98-0.94 (m, 3H), 0.54-0.50 (m, 1H); MS: m/z=362; HPLC retention time: 11.72 min.

### Examples 25a and 25b

### N-(1-((S)-1-phenylpropyl)-1H-pyrazol-4-yl)-3,4,5,5a,6,6a-hexahydrocyclopropa[e]indazole-1-carboxamide

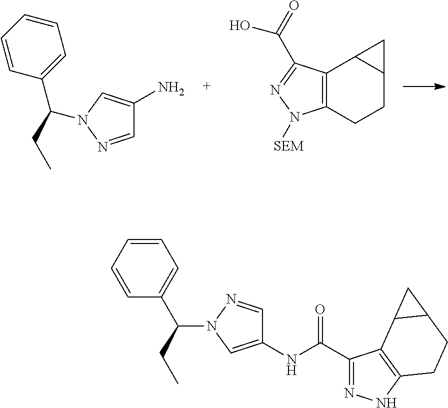

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 3-((2-(trimethylsilyl)ethoxy)methyl)-3,4,5,5a,6,6a-hexahydrocyclopropa[e]indazole-1-carboxylic acid (Example C19b) and 3-((4-amino-1H-pyrazol-1-yl)methyl)benzonitrile (Example A1) with (S)-1-(1-phenylpropyl)-1H-pyrazol-4-amine (Example A4). Also, the diastereomers were separated by preparative chiral HPLC instead of SFC.

Chiral HPLC conditions: ChiralPak IA (4.6×250 mm, 5 μm particle size); eluent=hexane (0.1% Et₃N):EtOH 50:50; 0.8 ml/min, 5.2 MPA, 25° C.

25a: ¹H NMR (400 MHz, CDCl₃) δ 8.53 (s, 1H), 8.16 (s, 1H), 7.54-7.49 (d, 1H), 7.32-7.28 (d, 4H), 5.16 (s, 1H), 3.48-3.46 (d, 1H), 3.14-3.04 (t, 3H), 2.45-2.46 (d, 1H), 2.22-2.20 (d, 1H), 1.29-1.24 (d, 2H), 1.07-0.89 (m, 3H), 0.63 (s, 1H), 0.17 (s, 1H); MS: m/z=362; HPLC retention time: 9.47 min.

25b: ¹H NMR (400 MHz, CDCl₃) δ 8.53 (s, 1H), 8.16 (s, 1H), 7.54-7.49 (d, 1H), 7.32-7.28 (d, 4H), 5.16 (s, 1H), 3.48-3.46 (d, 1H), 3.14-3.04 (t, 3H), 2.45-2.46 (d, 1H), 2.22-2.20 (d, 1H), 1.29-1.24 (d, 2H), 1.07-0.89 (m, 3H), 0.63 (s, 1H), 0.17 (s, 1H); MS: m/z=362; HPLC retention time: 10.67 min.

### Examples 26a-d

### N-(1-(3-(dimethylamino)-1-phenylpropyl)-1H-pyrazol-4-yl)-3,4,5,5a,6,6a-hexahydrocyclopropa[e]indazole-1-carboxamide

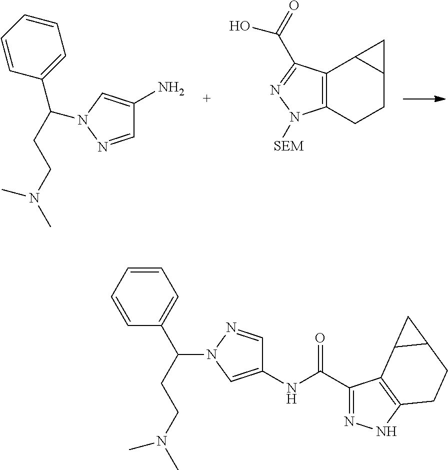

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 3-((2-(trimethylsilyl)ethoxy)methyl)-3,4,5,5a,6,6a-hexahydrocyclopropa[e]indazole-1-carboxylic acid (Example C19b) and 3-((4-amino-1H-pyrazol-1-yl)methyl)benzonitrile (Example A1) with 1-(3-(dimethylamino)-1-phenylpropyl)-1H-pyrazol-4-amine (Example A3). Also, the stereoisomers were separated by preparative chiral HPLC instead of SFC.

Chiral HPLC Conditions: ChiralPak IC (4.6×250 mm, 5 μm particle size); eluent=hexane (0.1% Et₃N):EtOH 80:20; 1.0 ml/min, 4.4 MPA, 25° C.

26a: ¹H-NMR (CDCl₃, 400 MHz, ppm): δ 9.85 (s, 1H), 8.54 (s, 1H), 8.11 (s, 1H), 7.59-7.54 (d, 1H), 7.35-7.26 (m, 5H), 5.44-5.41 (m, 1H), 3.48-3.44 (d, 1H), 3.07-2.96 (m, 3H), 2.70-2.65 (t, 1H), 2.40-2.32 (m, 9H), 1.28-1.24 (d, 2H), 0.64-0.59 (m, 1H), 0.11-0.05 (s, 1H); MS: m/z=405 (M+H); HPLC retention time: 14.86 min.

26b: ¹H-NMR (CDCl₃, 400 MHz, ppm): δ 9.86 (s, 1H), 8.57 (s, 1H), 8.10 (s, 1H), 7.58-7.54 (d, 1H), 7.41-7.27 (m, 5H), 5.44-5.40 (m, 1H), 3.48-3.44 (d, 1H), 3.07-2.96 (m, 3H), 2.68 (s, 1H), 2.31 (s, 9H), 1.28 (s, 2H), 0.64-0.59 (m, 1H), 0.096-0.070 (t, 1H); MS: m/z=405 (M+H); HPLC retention time: 17.16 min.

26c: ¹H-NMR (CDCl₃, 400 MHz, ppm): δ 9.84 (s, 1H), 8.53 (s, 1H), 8.10 (s, 1H), 7.58-7.50 (t, 1H), 7.36-7.28 (m, 5H), 5.44-5.40 (m, 1H), 3.48-3.44 (d, 1H), 3.07-2.96 (m, 3H), 2.70-2.68 (d, 1H), 2.38-2.14 (m, 9H), 1.29 (s, 2H), 0.64-0.59 (m, 1H), 0.11-0.069 (m, 1H); MS: m/z=405 (M+H); HPLC retention time: 22.63 min.

26d: ¹H-NMR (CDCl₃, 400 MHz, ppm): δ 9.84 (s, 1H), 8.54 (s, 1H), 8.09 (s, 1H), 7.60-7.54 (d, 1H), 7.35-7.28 (m, 5H), 5.45-5.41 (m, 1H), 3.48-3.44 (d, 1H), 3.07-2.96 (m, 3H), 2.74-2.56 (d, 1H), 2.38-2.20 (m, 9H), 1.28 (s, 2H), 0.64-0.59 (m, 1H), 0.13-0.084 (m, 1H); MS: m/z=405 (M+H); HPLC retention time: 28.51 min.

### Examples 27a-d

### N-(1-((l-methyl-1H-pyrazol-4-yl)(phenyl)methyl)-1H-pyrazol-4-yl)-1,4,4a,5,5a,6-hexahydrocyclopropa[f]indazole-3-carboxamide

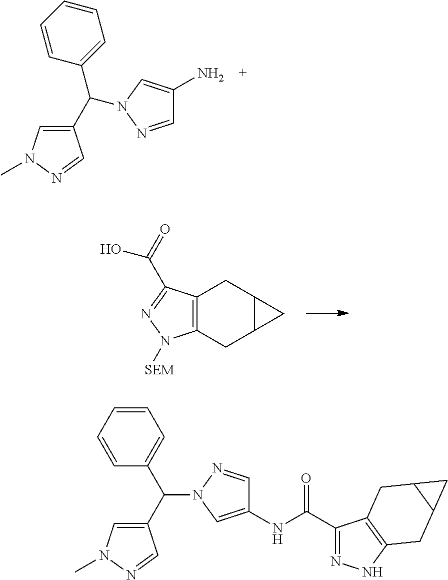

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1,4,4a,5,5a,6-hexahydrocyclopropa[f]indazole-3-carboxylic acid (Example C19a) and 3-((4-amino-1H-pyrazol-1-yl)methyl)benzonitrile (Example A1) with 1-((1-methyl-1H-pyrazol-4-yl)(phenyl)methyl)-1H-pyrazol-4-amine (Example A49 Also, the stereoisomers were separated by preparative chiral HPLC instead of SFC.

Chiral HPLC Conditions (27a/b): ChiralPak IC (4.6×250 mm, 5 μm particle size); eluent=hexane (0.1% Et₃N):EtOH 70:30; 1.0 ml/min, 5.4 MPA, 25° C.

27a: ¹H-NMR (300 MHz, CD₃OD, ppm) δ 8.04 (s, 1H), 7.77 (s, 1H), 7.49 (s, 1H), 7.30-7.40 (m, 4H), 7.20-7.22 (d, J=6.6 Hz, 2H), 6.72 (s, 1H), 3.89 (s, 3H), 2.71-2.78 (m, 1H), 2.21-2.38 (m, 3H), 1.77-1.79 (m, 1H), 1.48 (s, 1H), 0.96-0.97 (d, J=5.1 Hz, 1H), 0.45 (s, 1H); MS: m/z=414 (M+H); HPLC retention time: 15.05 min.

27b: ¹H-NMR (300 MHz, CD₃OD, ppm) δ 8.04 (s, 1H), 7.73 (s, 1H), 7.49 (s, 1H), 7.30-7.41 (m, 4H), 7.20-7.22 (m, 2H), 6.71 (s, 1H), 3.89 (s, 3H), 2.71-2.78 (m, 1H), 2.17-2.42 (m, 3H), 1.76-1.88 (m, 1H), 1.45-1.55 (m, 1H), 0.90-1.01 (m, 1H), 0.45 (m, 1H); MS: m/z=414 (M+H); HPLC retention time: 17.66 min.

Chiral HPLC Conditions (27c/d): ChiralPak IA (4.6×150 mm, 5 μm particle size); eluent=MTBE (0.2% DEA):EtOH 90:10; 1.0 ml/min, 6.3 MPA, 25° C.

27c: ¹H-NMR (300 MHz, CD₃OD, ppm) δ 8.03 (s, 1H), 7.72 (s, 1H), 7.49 (s, 1H), 7.32-7.41 (m, 4H), 7.20-7.22 (m, 2H), 6.71 (s, 1H), 3.89 (s, 3H), 2.71-2.78 (m, 1H), 2.17-2.38 (m, 3H), 1.76-1.88 (m, 1H), 1.48 (s, 1H), 0.90-1.00 (m, 1H), 0.50 (s, 1H); MS: m/z=414 (M+H); HPLC retention time: 5.22 min.

27d: ¹H-NMR (300 MHz, CD₃OD, ppm) δ 8.04 (s, 1H), 7.65 (s, 1H),7.49 (s, 1H), 7.31-7.38 (m, 4H), 7.20-7.22 (m, 2H), 6.77 (s, 1H), 3.89 (s, 3H), 2.71-2.78 (m, 1H), 2.17-2.38 (m, 3H), 1.81-1.84 (m, 1H), 1.50-1.57 (m, 1H), 0.90-1.00 (m, 1H), 0.50 (s, 1H); MS: m/z=414 (M+H); HPLC retention time: 6.42 min.

### Examples 28a-d

### N-(1-(3-(dimethylamino)-1-phenylpropyl)-1H-pyrazol-4-yl)-1,4,4a,5,5a,6-hexahydrocyclopropa[f]indazole-3-carboxamide

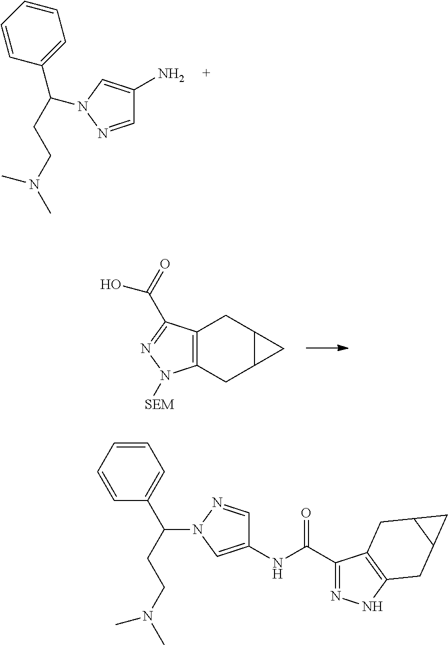

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1,4,4a,5,5a,6-hexahydrocyclopropa[f]indazole-3-carboxylic acid (Example C19a) and 3-((4-amino-1H-pyrazol-1-yl)methyl)benzonitrile (Example A1) with 1-(3-(dimethylamino)-1-phenylpropyl)-1H-pyrazol-4-amine (Example A3). Also, the stereoisomers were separated by preparative chiral HPLC instead of SFC.

Chiral HPLC Conditions: ChiralPak AD-H (4.6×250 mm, 5 μm particle size); eluent=hexane (0.1% Et₃N):EtOH 50:50; 1.0 ml/min, 6.9 MPA, 25° C.

28a: ¹H-NMR (CD₃OD, 400 MHz, ppm) δ 8.13 (s, 1H), 7.73 (s, 1H), 7.37-7.30 (m, 5H), 5.46-5.42 (m, 1H), 2.78-2.62 (m, 2H), 2.47-2.40 (m, 4H), 2.39-2.29 (m, 6H), 2.24-2.19 (m, 2H), 1.96-1.80 (m, 1H), 1.52 (s, 1H), 0.99 (m, 1H), 0.55 (s, 1H); MS: m/z=405 (M+H); HPLC retention time: 11.06 min.

28b: ¹H-NMR (CD₃OD, 400 MHz, ppm) δ 8.13 (s, 1H), 7.73 (s, 1H), 7.37-7.30 (m, 5H), 5.46-5.42 (m, 1H), 2.77-2.72 (m, 2H), 2.68-2.62 (m, 4H), 2.42-2.30 (m, 6H), 2.24-2.13 (m, 2H), 1.86-1.83 (m, 1H), 1.50 (s, 1H), 0.99 (d, 1H), 0.53 (s, 1H); MS: m/z=405 (M+H); HPLC retention time: 34.28 min.

28c: ¹H-NMR (CD₃OD, 400 MHz, ppm) δ 8.13 (s, 1H), 7.72 (s, 1H), 7.37-7.30 (m, 5H), 5.46-5.42 (m, 1H), 2.78-2.72 (m, 2H), 2.69-2.62 (m, 4H), 2.41-2.29 (m, 6H), 2.24-2.18 (m, 2H), 1.86-1.82 (m, 1H), 1.50 (s, 1H), 0.99 (m, 1H), 0.52 (s, 1H); MS: m/z=405 (M+H); HPLC retention time: 10.72 min.

28d: ¹H-NMR (CD₃OD, 400 MHz, ppm) δ 8.13 (s, 1H), 7.72 (s, 1H), 7.37-7.30 (m, 5H), 5.46-5.42 (m, 1H), 2.78-2.72 (m, 2H), 2.67-2.61 (m, 4H), 2.44-2.29 (m, 6H), 2.22-2.19 (m, 2H), 1.86-1.80 (m, 1H), 1.50 (s, 1H), 1.00 (m, 1H), 0.52 (s, 1H); MS: m/z=405 (M+H); HPLC retention time: 17.34 min.

### Examples 29a and 29b

### N-(1-benzyl-1H-pyrazol-4-yl)-5a-methyl-1,4,4a,5,5a,6-hexahydrocyclopropa[f]indazole-3-carboxamide

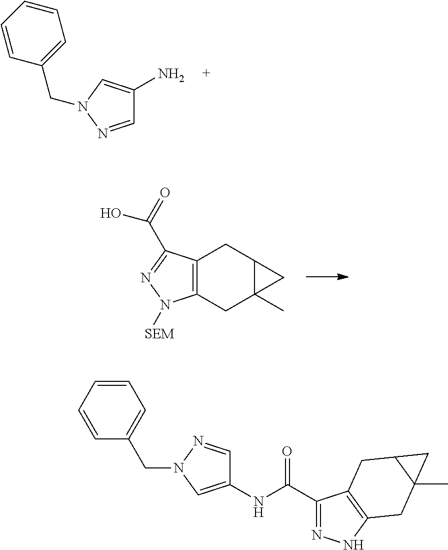

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5a-methyl-1-((2-(trimethylsilyl)ethoxy)methyl)-1,4,4a,5,5a,6-hexahydrocyclopropa[f]indazole-3-carboxylic acid (Example C31) and 3-((4-amino-1H-pyrazol-1-yl)methyl)benzonitrile (Example A1) with 1-benzyl-1H-pyrazol-4-amine (Example A2). Also, the stereoisomers were separated by preparative chiral HPLC instead of SFC.

Chiral HPLC Conditions: ChiralPak AD-H (4.6×150 mm, 5 μm particle size); eluent=hexane (0.1% Et₃N):EtOH 60:40; 1.0 ml/min, 4.4 MPA, 25° C.

29a: ¹H-NMR (300 MHz, CDCl₃, ppm) δ 8.58 (s, 1H), 8.05 (s, 1H), 7.56 (s, 1H), 7.32-7.38 (m, 3H), 7.24-7.30 (m, 2H), 5.29 (s, 2H), 3.37-3.42 (m, 1H), 2.99-3.07 (m, 2H), 2.70-2.75 (m, 1H), 1.26 (s, 3H), 1.05-1.13 (m, 1H), 0.38-0.43 (m, 1H), 0.22-0.25 (m, 1H); MS: m/z=348 (M+H); HPLC retention time: 6.55 min.

29b: ¹H-NMR (300 MHz, CDCl₃, ppm) δ 8.58 (s, 1H), 8.05 (s, 1H), 7.55 (s, 1H), 7.32-7.37 (m, 3H), 7.24-7.30 (m, 2H), 5.28 (s, 2H), 3.36-3.42 (m, 1H), 2.97-3.07 (m, 2H), 2.69-2.74 (m, 1H), 1.25-1.40 (m, 3H), 1.07-1.13 (m, 1H), 0.38-0.42 (m, 1H), 0.22-0.24 (m, 1H); MS: m/z=348 (M+H); HPLC retention time: 9.29 min.

### Examples 30a and 30b

### N-(1-(3-(dimethylamino)-1-(3-(trifluoromethyl)phenyl)propyl)-1H-pyrazol-4-yl)-6,6-dimethyl-4,5,6,7-tetrahydro-1H-indazole-3-carboxamide

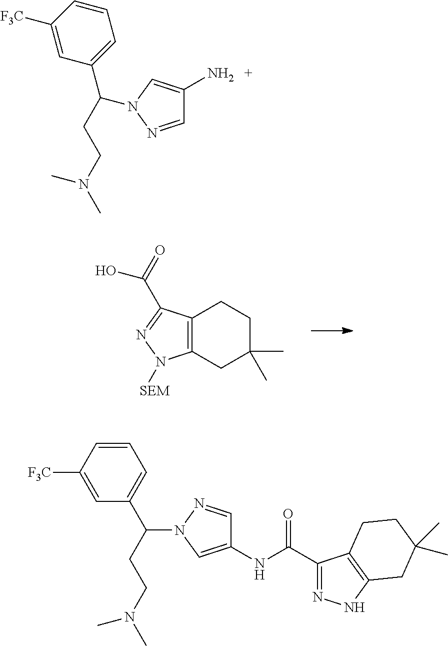 [SMILES: CN(C)CCC(c1cccc(C(F)(F)F)c1)n1cc(N)cn1 ; CC1(C)CCc2c(C(=O)O)nn(COCC[Si](C)(C)C)c2C1 ; CN(C)CCC(c1cccc(C(F)(F)F)c1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(3-(dimethylamino)-1-(3-(trifluoromethyl)phenyl)propyl)-1H-pyrazol-4-amine (Example A52). Also, the stereoisomers were separated by preparative chiral HPLC instead of SFC.

Chiral HPLC Conditions: Venusil chiral OD-H (4.6×250 mm, 5 μm particle size); eluent=hexane (0.1% Et₃N):EtOH 90:10; 1.0 ml/min, 9.5 MPA, 25° C.

30a: ¹H-NMR (CD₃OD, 300 MHz, ppm) δ 8.15 (1H, s), 7.67-7.75 (1H, s), 7.53-7.63 (4H, m), 5.53-5.58 (1H, m), 2.79-2.81 (2H, t), 2.71-2.73 (1H, t), 2.40-2.44 (3H, m), 2.29-2.36 (7H, s), 1.55-1.59 (2H, t), 1.24-1.30 (1H, s), 1.03 (6H, s); MS: m/z=489 (M+H); HPLC retention time: 9.33 min.

30b: ¹H-NMR (CD₃OD, 300 MHz, ppm) δ 8.15 (1H, s), 7.75 (1H, s), 7.53-7.75 (4H, m), 5.53-5.58 (1H, m), 2.72-2.79 (2H, t), 2.30-2.50 (11H, m), 1.55-1.59 (2H, t), 1.26-1.31 (1H, t), 1.03 (6H, s); MS: m/z=489 (M+H); HPLC retention time: 11.27 min.

### Example 31

### N-(1-benzyl-1H-pyrazol-4-yl)-4,5,6,7-tetrahydro-1H-4,6-methanoindazole-3-carboxamide

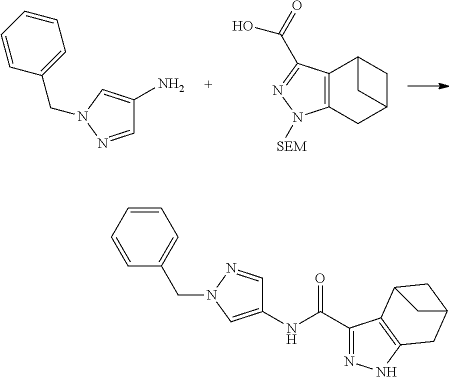 [SMILES: Nc1cnn(Cc2ccccc2)c1 ; C[Si](C)(C)CCOCn1nc(C(=O)O)c2c1CC1CC2C1 ; O=C(Nc1cnn(Cc2ccccc2)c1)c1n[nH]c2c1C1CC(C2)C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-4,5,6,7-tetrahydro-1H-4,6-methanoindazole-3-carboxylic acid (Example C32) and 3-((4-amino-1H-pyrazol-1-yl)methyl)benzonitrile (Example A1) with 1-benzyl-1H-pyrazol-4-amine (Example A2).

31: ¹H-NMR (400 MHz, CD₃OD, ppm): δ 8.06 (s, 1H), 7.69 (s, 1H), 7.60-7.26 (m, 5H), 5.41-5.01 (m, 2H), 3.64 (s, 1H), 3.01-3.00 (d, J=2.8, 2H), 2.89-2.88 (m, 1H), 2.68 (s, 2H), 1.63-1.49 (m, 2H); MS: m/z=334 (M+H).

### Example 32

### N-(1-benzyl-1H-pyrazol-4-yl)-4,5,6,7-tetrahydro-1H-5,7-methanoindazole-3-carboxamide

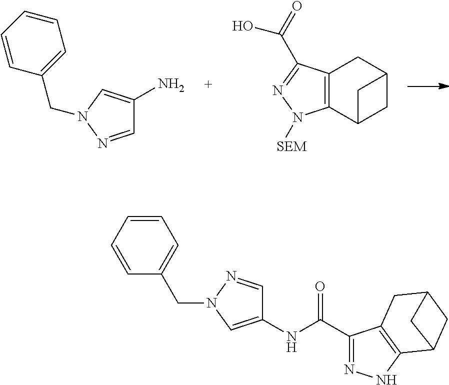 [SMILES: Nc1cnn(Cc2ccccc2)c1 ; C[Si](C)(C)CCOCn1nc(C(=O)O)c2c1C1CC(C2)C1 ; O=C(Nc1cnn(Cc2ccccc2)c1)c1n[nH]c2c1CC1CC2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-4,5,6,7-tetrahydro-1H-5,7-methanoindazole-3-carboxylic acid (Example C33) and 3-((4-amino-1H-pyrazol-1-yl)methyl)benzonitrile (Example A1) with 1-benzyl-1H-pyrazol-4-amine (Example A2).

32: ¹H-NMR (300 MHz, CD₃OD, ppm) δ 8.09 (s, 1H), 7.70 (s, 1H), 7.77-7.22 (m, 5H), 5.39-5.01 (m, 2H), 3.23 (s, 1H), 3.21-3.03 (m, 2H), 2.89-2.87 (m, 1H), 2.62 (s, 2H), 1.63-1.51 (m, 1H); MS: m/z=334 (M+H).

### Examples 33a and 33b

### N-(1-(1-(3-chlorophenyl)-3-(dimethylamino)propyl)-1H-pyrazol-4-yl)-6,6-dimethyl-4,5,6,7-tetrahydro-1H-indazole-3-carboxamide

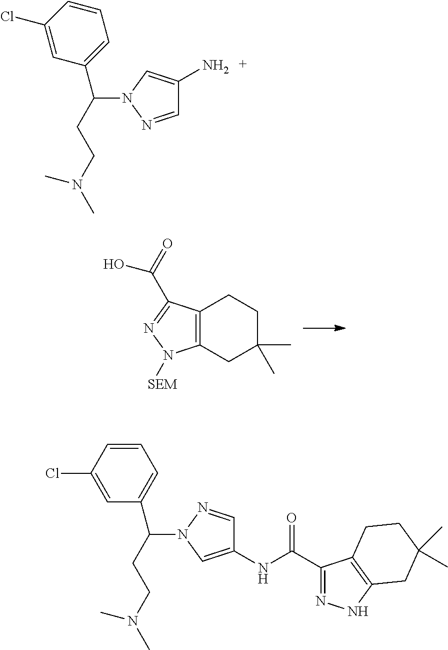

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(3-chlorophenyl)-3-(dimethylamino)propyl)-1H-pyrazol-4-amine (Example A51). Also, the stereoisomers were separated by preparative chiral HPLC instead of SFC.

Chiral HPLC Conditions: Venusil chiral OD-H (4.6×250 mm, 5 μm particle size); eluent=hexane (0.1% Et₃N):EtOH 90:10; 1.0 ml/min, 5.6 MPA, 25° C.

33a: ¹H-NMR (300 mHz, CD₃OD, ppm) δ 8.13 (s, 1H), 7.74 (s, 1H), 7.38-7.28 (m, 4H), 5.47-5.42 (m, 1H), 2.81-2.77 (t, J=12.6, 2H), 2.69-2.61 (m, 1H), 2.58 (s, 1H), 2.44-2.35 (m, 2H), 2.34 (s, 9H), 1.59-1.55 (t, J=12.9, 2H), 1.04 (s, 6H); MS: m/z=456 (M+H); HPLC retention time: 13.35 min.

33b: (400 mHz, CD₃OD, ppm) δ 8.14 (s, 1H), 7.74 (s, 1H), 7.39-7.29 (m, 4H), 5.47-5.43 (m, 1H), 2.81-2.78 (t, J=12.8, 2H), 2.68-2.60 (m, 1H), 2.45 (s, 2H), 2.42-2.31 (m, 2H), 2.29 (s, 6H), 2.22 (s, 1H), 1.59-1.56 (t, J=13.2, 2H), 1.04 (s, 6H); MS: m/z=456 (M+H); HPLC retention time: 17.71 min.

### Examples 34a and 34b

### N-(1-(azetidin-3-yl(phenyl)methyl)-1H-pyrazol-4-yl)-6,6-dimethyl-4,5,6,7-tetrahydro-1H-indazole-3-carboxamide

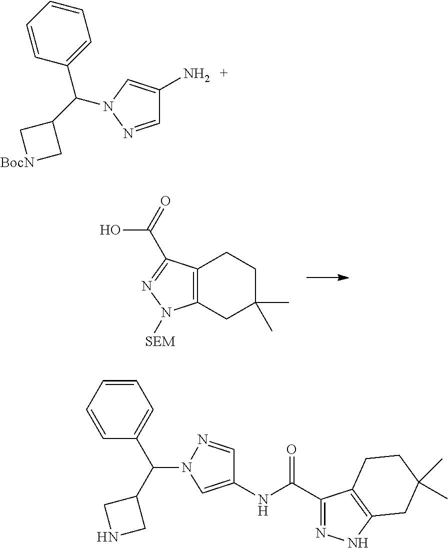

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with tert-butyl 3-((4-amino-1H-pyrazol-1-yl)(phenyl)methyl)azetidine-1-carboxylate (Example A20). SFC conditions: Chiralpak IC (4.6×50 mm, 5 μm particle size) at 45% methanol w/0.1% NH₄OH; 5 mL/min, 120 bars, 40° C.

34a: ¹H NMR (400 MHz, DMSO-d₆) δ 12.82 (s, 1H), 10.08 (s, 1H), 8.06 (s, 1H), 7.62 (s, 1H), 7.37-7.24 (m, 5H), 5.62 (d, J=11.1, 1H), 3.71-3.59 (m, 1H), 3.48 (t, J=7.7, 1H), 3.43-3.19 (m, 3H), 2.65 (t, J=6.2, 2H), 2.38 (s, 2H), 1.46 (t, J=6.2, 2H), 0.96 (s, 6H); MS: m/z=405 (M+H); SFC retention time: 0.42 min.

34b: ¹H NMR (400 MHz, DMSO-d₆) δ 12.78 (s, 1H), 10.02 (s, 1H), 8.05 (s, 1H), 7.62 (s, 1H), 7.40-7.21 (m, 5H), 5.61 (d, J=11.0 Hz, 1H), 3.70-3.58 (m, 1H), 3.46 (t, J=7.6 Hz, 1H), 3.41-3.18 (m, 3H), 2.66 (t, J=6.2 Hz, 2H), 2.38 (s, 2H), 1.46 (t, J=6.3 Hz, 2H), 0.96 (s, 6H); MS: m/z=405 (M+H); SFC retention time: 0.59 min.

### Examples 35a-d

### N-(1-((1-methyl-1H-pyrazol-4-yl)(phenyl)methyl)-1H-pyrazol-4-yl)-3,4,5,5a,6,6a-hexahydro cyclopropa[e]indazole-1-carboxamide

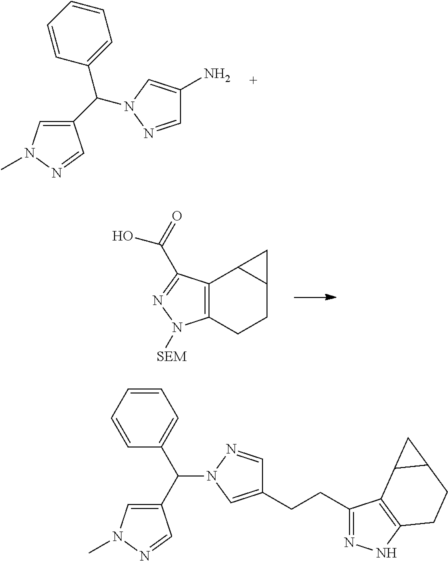

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 3-((2-(trimethylsilyl)ethoxy)methyl)-3,4,5,5a,6,6a-hexahydrocyclopropa[e]indazole-1-carboxylic acid (Example C19b) and 3-((4-amino-1H-pyrazol-1-yl)methyl)benzonitrile (Example A1) with 1-((1-methyl-1H-pyrazol-4-yl)(phenyl)methyl)-1H-pyrazol-4-amine (Example A49). Also, the stereoisomers were separated by preparative chiral HPLC instead of SFC.

Chiral HPLC Conditions: Venusil chiral OD-H (4.6×250 mm, 5 μm particle size); eluent=hexane (0.1% Et₃N):EtOH 50:50; 0.8 ml/min, 7.8 MPA, 25° C.

35a: ¹H-NMR (300 MHz, CD₃OD, ppm) δ 8.01 (s, 1H), 7.71 (s, 1H), 7.40-7.49 (d, 1H, J=24.9), 7.30-7.40 (m, 4H), 7.19-7.21 (m, 2H), 6.71 (s, 1H), 3.89 (s, 3H), 3.29-3.33 (m, 1H), 2.92-3.07 (m, 3H), 1.26-1.31 (m, 2H), 0.56-0.63 (m, 1H), 0.01-0.05 (m, 1H); MS: m/z=414 (M+H); HPLC retention time: 21.87 min.

35b: ¹H-NMR (300 MHz, CD₃OD, ppm) δ 8.01 (s, 1H), 7.70 (s, 1H), 7.48 (s, 1H), 7.30-7.39 (m, 4H), 7.19-7.21 (m, 2H), 6.71 (s, 1H), 3.89 (s, 3H), 2.92-3.07 (m, 3H), 1.19-1.37 (m, 3H), 0.62-0.63 (m, 1H), 0.01-0.09 (m, 1H); MS: m/z=414 (M+H); HPLC retention time: 27.88 min.

35c: ¹H-NMR (400 MHz, CD₃OD, ppm) δ 8.02 (s, 1H), 7.71 (s, 1H), 7.49 (s, 1H), 7.31-7.39 (m, 4H), 7.19-7.21 (d, 2H, J=7.2), 6.71 (s, 1H), 3.89 (s, 3H), 2.93-3.06 (m, 3H), 1.27-1.31 (m, 3H), 0.57-0.62 (m, 1H), 0.01-0.04 (m, 1H); MS: m/z=414 (M+H); HPLC retention time: 31.76 min.

35d: ¹H-NMR (400 MHz, CD₃OD, ppm) δ 8.01 (s, 1H), 7.71 (s, 1H), 7.49 (s, 1H), 7.31-7.39 (m, 4H), 7.19-7.21 (d, 2H, J=7.2), 6.71 (s, 1H), 3.89 (s, 3H), 2.93-3.06 (m, 3H), 1.21-1.42 (m, 3H), 0.57-0.62 (m, 1H), 0.05-0.10 (m, 1H); MS: m/z=414 (M+H); HPLC retention time: 72.57 min.

### Examples 36a and 36b

### N-(1-(3-(dimethylamino)-1-(m-tolyl)propyl)-1H-pyrazol-4-yl)-6,6-dimethyl-4,5,6,7-tetrahydro-1H-indazole-3-carboxamide

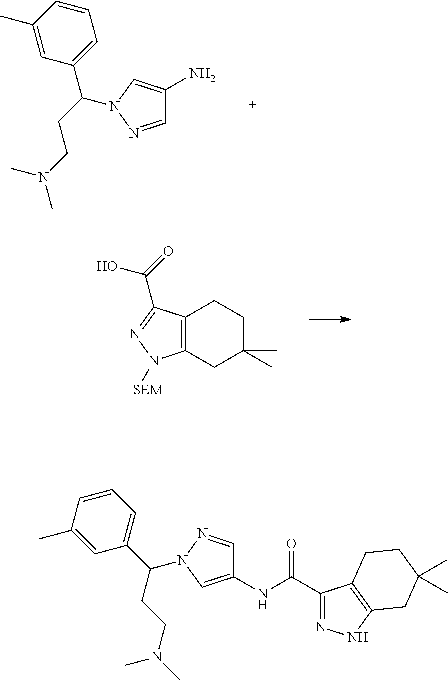

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(3-(dimethylamino)-1-(m-tolyl)propyl)-1H-pyrazol-4-amine (Example A50). Also, the stereoisomers were separated by preparative chiral HPLC instead of SFC.

Chiral HPLC Conditions: Venusil chiral OD-H (4.6×250 mm, 5 μm particle size); eluent=hexane (0.1% Et₃N):EtOH 90:10; 1.0 ml/min, 3.3 MPA, 25° C.

36a: ¹H-NMR (300 MHz, CD₃OD, ppm) δ 8.10 (s, 1H), 7.70 (s, 1H), 7.27-7.11 (m, 4H), 5.40-5.35 (m, 1H), 2.81-2.77 (t, J=12.3, 2H), 2.64-2.61 (m, 1H), 2.57 (s, 5H), 2.54 (s, 7H), 1.59-1.32 (m, 2H), 1.04 (s, 6H); MS: m/z=435 (M+H); HPLC retention time: 14.10 min.

36b: ¹H-NMR (300 MHz, CD₃OD, ppm) δ 8.10 (s, 1H), 7.70 (s, 1H), 7.26-7.11 (m, 4H), 5.40-5.35 (m, 1H), 2.81-2.77 (t, J=12.6, 2H), 2.70-2.61 (m, 1H), 2.57 (s, 5H), 2.54 (s, 7H), 1.60-1.33 (m, 2H), 1.04 (s, 6H); MS: m/z=435 (M+H); HPLC retention time: 21.73 min.

### Examples 37a and 37b

### N-(1-((1-acetylazetidin-3-yl)(phenyl)methyl)-1H-pyrazol-4-yl)-6,6-dimethyl-4,5,6,7-tetrahydro-1H-indazole-3-carboxamide

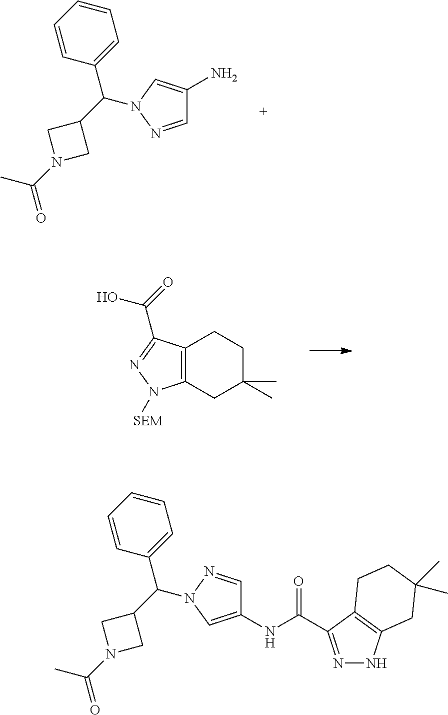 [SMILES: CC(=O)N1CC(C(c2ccccc2)n2cc(N)cn2)C1 ; CC1(C)CCc2c(C(=O)O)nn(COCC[Si](C)(C)C)c2C1 ; CC(=O)N1CC(C(c2ccccc2)n2cc(NC(=O)c3n[nH]c4c3CCC(C)(C)C4)cn2)C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(3-((4-amino-1H-pyrazol-1-yl)(phenyl)methyl)azetidin-1-yl)ethanone (Example A21). SFC conditions: (S,S)-Whelk-O1 (4.6×50 mm, 5 μm particle size) at 45% methanol w/0.1% NH₄OH; 5 mL/min, 120 bars, 40° C.

37a: ¹H NMR (400 MHz, DMSO-d₆) δ 12.81 (s, 1H), 10.12 (s, 1H), 8.07 (s, 1H), 7.66 (s, 1H), 7.43-7.27 (m, 5H), 5.67 (dd, J=11.0, 4.0 Hz, 1H), 4.12 (dt, J=32.4, 8.3 Hz, 1H), 3.88-3.74 (m, 2H), 3.71-3.60 (m, 1H), 3.58-3.46 (m, 1H), 2.65 (t, J=6.2 Hz, 2H), 2.38 (s, 2H), 1.72 (d, J=4.7 Hz, 3H), 1.46 (t, J=6.2 Hz, 2H), 0.96 (s, 6H); MS: m/z=447 (M+H); SFC retention time: 0.85 min.

37b: ¹H NMR (400 MHz, DMSO-d₆) δ 12.81 (s, 1H), 10.12 (s, 1H), 8.07 (s, 1H), 7.66 (s, 1H), 7.43-7.27 (m, 5H), 5.67 (dd, J=10.9, 4.1 Hz, 1H), 4.12 (dt, J=32.5, 8.4 Hz, 1H), 3.88-3.74 (m, 2H), 3.72-3.60 (m, 1H), 3.57-3.47 (m, 1H), 2.65 (t, J=6.2 Hz, 2H), 2.38 (s, 2H), 1.72 (d, J=4.7 Hz, 3H), 1.46 (t, J=6.2 Hz, 2H), 0.96 (s, 6H); MS: m/z=447 (M+H); SFC retention time: 1.23 min.

### Examples 38a and 38b

### 6,6-dimethyl-N-(1-((1-methylazetidin-3-yl)(phenyl)methyl)-1H-pyrazol-4-yl)-4,5,6,7-tetrahydro-1H-indazole-3-carboxamide

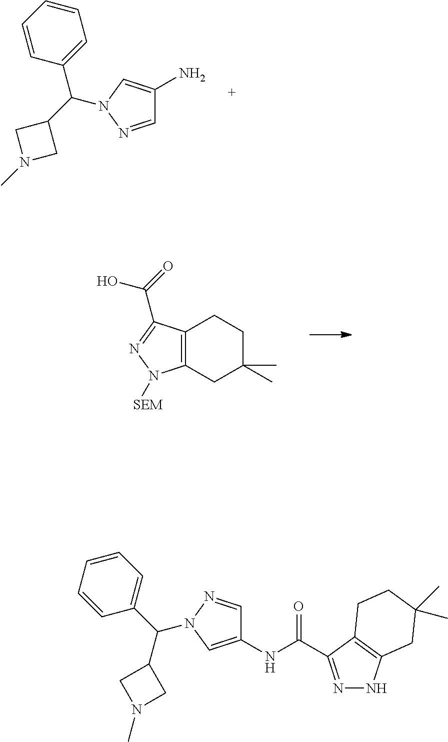 [SMILES: CN1CC(C(c2ccccc2)n2cc(N)cn2)C1 ; CC1(C)CCc2c(C(=O)O)nn(COCC[Si](C)(C)C)c2C1 ; CN1CC(C(c2ccccc2)n2cc(NC(=O)c3n[nH]c4c3CCC(C)(C)C4)cn2)C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-((1-methylazetidin-3-yl)(phenyl)methyl)-1H-pyrazol-4-amine (Example A22).

SFC conditions: Lux Cellulose-2 (4.6×50 mm, 5 μm particle size) at 45% methanol w/0.1% NH₄OH; 5 mL/min, 120 bars, 40° C.

38a: ¹H NMR (400 MHz, DMSO-d₆) δ 12.81 (s, 1H), 10.08 (s, 1H), 8.06 (s, 1H), 7.62 (s, 1H), 7.38-7.24 (m, 5H), 5.57 (d, J=11.0 Hz, 1H), 3.48-3.14 (m, 3H), 2.87 (dt, J=17.6, 6.6 Hz, 2H), 2.65 (t, J=6.5 Hz, 2H), 2.38 (s, 2H), 2.22 (s, 3H), 1.46 (t, J=6.5 Hz, 2H), 0.96 (s, 6H); MS: m/z=419 (M+H); SFC retention time: 0.87 min.

38b: ¹H NMR (400 MHz, DMSO-d₆) δ 12.81 (s, 1H), 10.08 (s, 1H), 8.06 (s, 1H), 7.63 (s, 1H), 7.38-7.24 (m, 5H), 5.57 (d, J=11.0 Hz, 1H), 3.55-3.13 (m, 3H), 2.87 (dt, J=17.9, 6.6 Hz, 2H), 2.65 (t, J=6.5 Hz, 2H), 2.38 (s, 2H), 2.23 (s, 3H), 1.46 (t, J=6.4 Hz, 2H), 0.96 (s, 6H); MS: m/z=419 (M+H); SFC retention time: 1.21 min.

### Examples 39a and 39b

### 6,6-dimethyl-N-(1-(3-(methylamino)-1-phenylpropyl)-1H-pyrazol-4-yl)-4,5,6,7-tetrahydro-1H-indazole-3-carboxamide

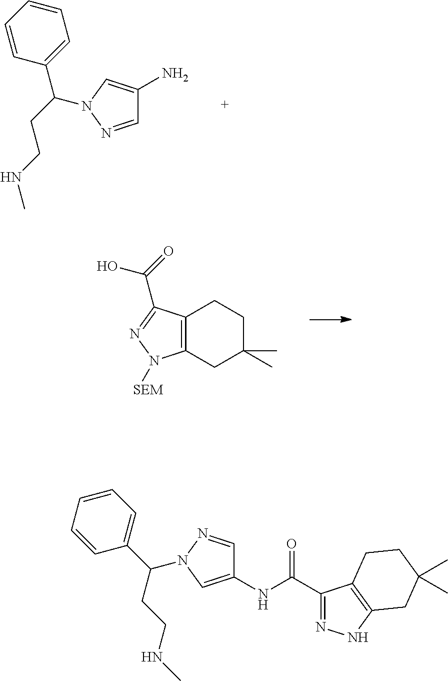 [SMILES: CNCCC(c1ccccc1)n1cc(N)cn1 ; CC1(C)CCc2c(C(=O)O)nn(COCC[Si](C)(C)C)c2C1 ; CNCCC(c1ccccc1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(3-(methylamino)-1-phenylpropyl)-1H-pyrazol-4-amine (Example A9).

SFC conditions: Lux Cellulose-4 (4.6×50 mm, 5 μm particle size) at 45% methanol w/0.1% NH₄OH; 5 mL/min, 120 bars, 40° C.

39a: ¹H NMR (400 MHz, DMSO-d₆) δ 13.00-12.66 (s, 1H), 10.14-9.87 (s, 1H), 8.12-8.05 (s, 1H), 7.68-7.64 (s, 1H), 7.38-7.21 (m, 5H), 5.55-5.44 (dd, J=8.9, 5.9 Hz, 1H), 2.69-2.62 (t, J=6.5 Hz, 2H), 2.48-2.39 (q, J=6.0 Hz, 1H), 2.39-2.37 (s, 2H), 2.30-2.27 (s, 3H), 2.27-2.15 (m, 1H), 1.50-1.42 (t, J=6.4 Hz, 2H), 0.98-0.94 (s, 7H). MS: m/z=407 (M+H); SFC retention time: 0.45 min.

39b: ¹H NMR (400 MHz, DMSO-d₆) δ 13.04-12.49 (s, 1H), 10.11-10.03 (s, 1H), 8.11-8.07 (s, 1H), 7.70-7.66 (s, 1H), 7.39-7.16 (m, 5H), 5.55-5.43 (dd, J=8.8, 5.7 Hz, 1H), 2.71-2.62 (t, J=6.3 Hz, 2H), 2.47-2.40 (m, 1H), 2.39-2.37 (s, 2H), 2.31-2.29 (s, 3H), 2.25-2.17 (m, 1H), 1.50-1.42 (t, J=6.4 Hz, 2H), 1.00-0.94 (s, 6H); MS: m/z=407 (M+H); SFC retention time: 0.45 min.

### Examples 40a and 40b

### 6,6-dimethyl-N-(1-(phenyl(piperidin-4-yl)methyl)-1H-pyrazol-4-yl)-4,5,6,7-tetrahydro-1H-indazole-3-carboxamide

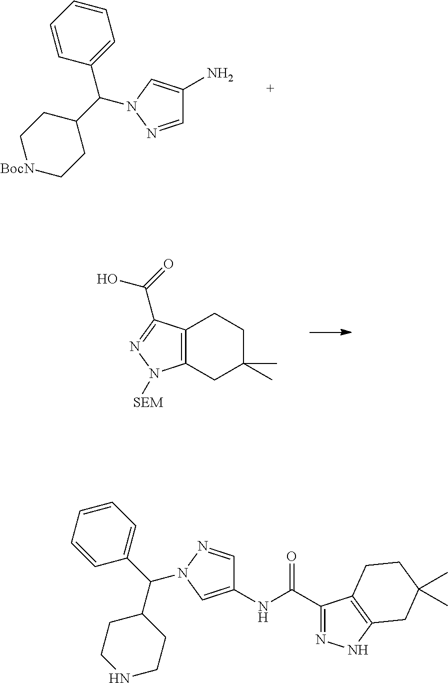 [SMILES: CC(C)(C)OC(=O)N1CCC(C(c2ccccc2)n2cc(N)cn2)CC1 ; CC1(C)CCc2c(C(=O)O)nn(COCC[Si](C)(C)C)c2C1 ; CC1(C)CCc2c(C(=O)Nc3cnn(C(c4ccccc4)C4CCNCC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with tert-butyl 4-((4-amino-1H-pyrazol-1-yl)(phenyl)methyl)piperidine-1-carboxylate (Example A24).

SFC conditions: Lux Cellulose-1 (4.6×50 mm, 5 μm particle size) at 30% methanol w/0.1% NH₄OH; 5 mL/min, 120 bars, 40° C.

40a: ¹H NMR (400 MHz, DMSO-d₆) δ 12.82 (s, 1H), 10.06 (s, 1H), 8.14 (s, 1H), 7.63 (s, 1H), 7.55-7.48 (m, 2H), 7.37-7.30 (m, 2H), 7.30-7.24 (m, 1H), 5.00 (d, J=10.7 Hz, 1H), 2.97-2.84 (m, 2H), 2.70-2.62 (m, 2H), 2.55-2.36 (m, 6H), 1.46 (t, J=6.3 Hz, 2H), 1.21-1.00 (m, 4H), 0.96 (s, 6H); MS: m/z=433 (M+H); SFC retention time: 0.43 min.

40b: ¹H NMR (400 MHz, DMSO-d₆) δ 12.83 (s, 1H), 10.07 (s, 1H), 8.14 (s, 1H), 7.63 (s, 1H), 7.55-7.48 (m, 2H), 7.37-7.30 (m, 2H), 7.30-7.24 (m, 1H), 4.99 (d, J=10.7 Hz, 1H), 2.97-2.83 (m, 2H), 2.66 (t, J=6.1 Hz, 2H), 2.55-2.31 (m, 6H), 1.46 (t, J=6.2 Hz, 2H), 1.22-1.00 (m, 4H), 0.96 (s, 6H); MS: m/z=433 (M+H); SFC retention time: 0.58 min.

### Examples 41a and 41b

### N-(1-(2-(dimethylamino)-1-phenylethyl)-1H-pyrazol-4-yl)-6,6-dimethyl-4,5,6,7-tetrahydro-1H-indazole-3-carboxamide

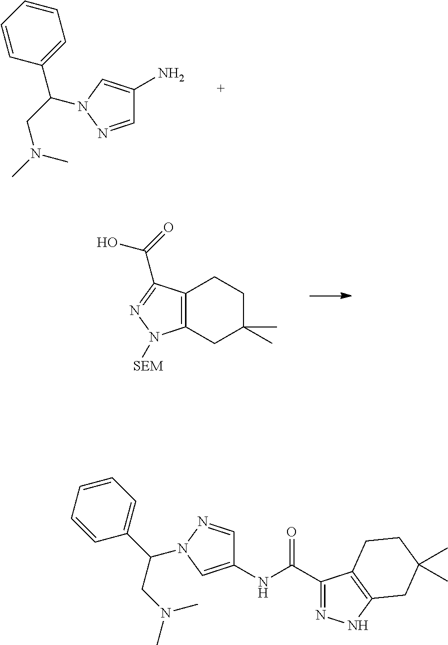 [SMILES: CN(C)CC(c1ccccc1)n1cc(N)cn1 ; CC1(C)CCc2c(C(=O)O)nn(COCC[Si](C)(C)C)c2C1 ; CN(C)CC(c1ccccc1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(2-(dimethylamino)-1-phenylethyl)-1H-pyrazol-4-amine (Example A5).

SFC conditions: Chiralpak AD (4.6×50 mm, 5 μm particle size) at 35% methanol w/0.1% NH₄OH; 5 mL/min, 120 bars, 40° C.

41a: ¹H NMR (400 MHz, DMSO-d₆) δ 12.89-12.74 (s, 1H), 10.16-9.97 (s, 1H), 8.18-8.15 (s, 1H), 7.64-7.60 (s, 1H), 7.39-7.22 (m, 5H), 5.66-5.44 (dd, J=9.0, 5.7 Hz, 1H), 3.28-3.20 (dd, J=12.8, 9.3 Hz, 1H), 2.82-2.74 (dd, J=12.9, 5.8 Hz, 1H), 2.69-2.63 (t, J=6.2 Hz, 2H), 2.40-2.36 (s, 2H), 2.19-2.16 (s, 6H), 1.50-1.43 (t, J=6.5 Hz, 2H), 0.99-0.93 (s, 6H); MS: m/z=407 (M+H); SFC retention time: 0.43 min.

41b: ¹H NMR (400 MHz, DMSO-d₆) δ 12.91-12.72 (s, 1H), 10.20-9.97 (s, 1H), 8.17-8.15 (s, 1H), 7.64-7.61 (s, 1H), 7.38-7.23 (m, 5H), 5.68-5.46 (dd, J=9.1, 5.8 Hz, 1H), 3.28-3.19 (dd, J=12.7, 9.4 Hz, 1H), 2.82-2.74 (dd, J=12.9, 5.7 Hz, 1H), 2.70-2.63 (t, J=6.3 Hz, 2H), 2.42-2.37 (s, 2H), 2.21-2.16 (s, 6H), 1.50-1.44 (t, J=6.3 Hz, 2H), 0.99-0.93 (s, 6H); MS: m/z=407 (M+H); SFC retention time: 0.58 min.

### Examples 42a and 42b

### N-(1-(3-amino-1-phenylpropyl)-1H-pyrazol-4-yl)-6,6-dimethyl-4,5,6,7-tetrahydro-1H-indazole-3-carboxamide

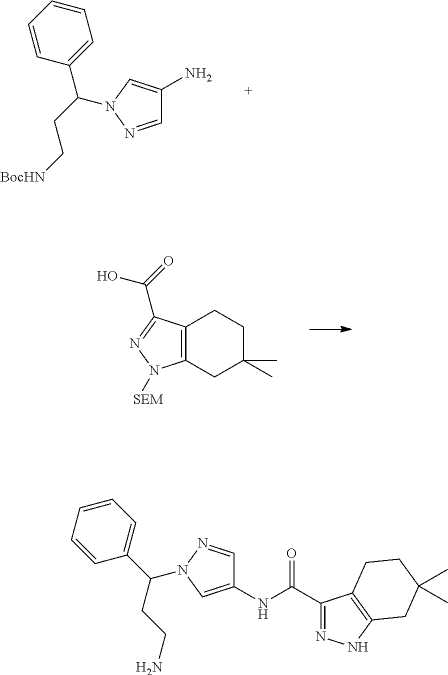 [SMILES: CC(C)(C)OC(=O)NCCC(c1ccccc1)n1cc(N)cn1 ; CC1(C)CCc2c(C(=O)O)nn(COCC[Si](C)(C)C)c2C1 ; CC1(C)CCc2c(C(=O)Nc3cnn(C(CCN)c4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with tert-butyl (3-(4-amino-1H-pyrazol-1-yl)-3-phenylpropyl)carbamate (Example A23).

SFC conditions: (S,S)-Whelk-O1 (4.6×50 mm, 5 μm particle size) at 45% methanol w/0.1% NH₄OH; 5 mL/min, 120 bars, 40° C.

42a: ¹H NMR (400 MHz, DMSO-d₆) δ 12.83 (s, 1H), 10.10 (s, 1H), 8.11 (s, 1H), 7.65 (s, 1H), 7.36-7.22 (m, 5H), 5.53 (t, J=6.8 Hz, 1H), 2.66 (t, J=6.0 Hz, 2H), 2.54-2.35 (m, 7H), 2.16-2.04 (m, 1H), 1.47 (t, J=6.2 Hz, 2H), 0.96 (s, 6H); MS: m/z=393 (M+H); SFC retention time: 0.51 min.

42b: ¹H NMR (400 MHz, DMSO-d₆) δ 12.83 (s, 1H), 10.10 (s, 1H), 8.11 (s, 1H), 7.65 (s, 1H), 7.39-7.22 (m, 5H), 5.53 (t, J=6.7 Hz, 1H), 2.66 (t, J=6.1 Hz, 2H), 2.55-2.35 (m, 7H), 2.17-2.04 (m, 1H), 1.47 (t, J=6.2 Hz, 2H), 0.96 (s, 6H); MS: m/z=393 (M+H); SFC retention time: 0.57 min.

### Examples 43a and 43b

### 6,6-dimethyl-N-(1-((1-methylpiperidin-4-yl)(phenyl)methyl)-1H-pyrazol-4-yl)-4,5,6,7-tetrahydro-1H-indazole-3-carboxamide

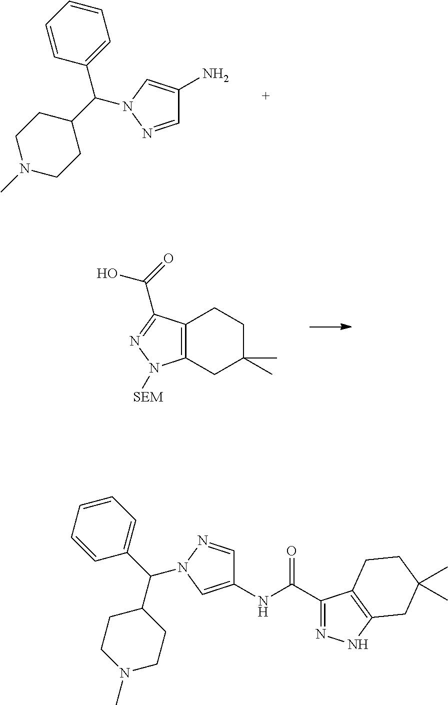 [SMILES: CN1CCC(C(c2ccccc2)n2cc(N)cn2)CC1 ; CC1(C)CCc2c(C(=O)O)nn(COCC[Si](C)(C)C)c2C1 ; CN1CCC(C(c2ccccc2)n2cc(NC(=O)c3n[nH]c4c3CCC(C)(C)C4)cn2)CC1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-((1-methylpiperidin-4-yl)(phenyl)methyl)-1H-pyrazol-4-amine (Example A25).

SFC conditions: Lux Cellulose-1 (4.6×50 mm, 5 μm particle size) at 30% methanol w/0.1% NH₄OH; 5 mL/min, 120 bars, 40° C.

43a: ¹H NMR (400 MHz, DMSO-d₆) δ 12.77 (s, 1H), 10.02 (s, 1H), 8.13 (s, 1H), 7.63 (s, 1H), 7.51 (d, J=7.5 Hz, 2H), 7.33 (t, J=7.4 Hz, 2H), 7.27 (t, J=7.2 Hz, 1H), 5.00 (d, J=10.8 Hz, 1H), 2.76-2.61 (m, 4H), 2.40-2.25 (m, 3H), 2.11 (s, 3H), 1.85-1.70 (m, 2H), 1.47 (t, J=6.3 Hz, 2H), 1.30-1.09 (m, 4H), 0.96 (s, 6H); MS: m/z=447 (M+H); SFC retention time: 0.39 min.

43b: ¹H NMR (400 MHz, DMSO-d₆) δ 12.77 (s, 1H), 10.02 (s, 1H), 8.13 (s, 1H), 7.63 (s, 1H), 7.51 (d, J=7.4 Hz, 2H), 7.33 (t, J=7.4 Hz, 2H), 7.27 (t, J=7.2 Hz, 1H), 5.00 (d, J=10.7 Hz, 1H), 2.77-2.62 (m, 4H), 2.40-2.26 (m, 3H), 2.12 (s, 3H), 1.87-1.73 (m, 2H), 1.47 (t, J=6.3 Hz, 2H), 1.31-1.09 (m, 4H), 0.96 (s, 6H); MS: m/z=447 (M+H); SFC retention time: 0.47 min.

### Examples 44a and 44b

### N-(1-((4-fluoropiperidin-4-yl)(phenyl)methyl)-1H-pyrazol-4-yl)-6,6-dimethyl-4,5,6,7-tetrahydro-1H-indazole-3-carboxamide

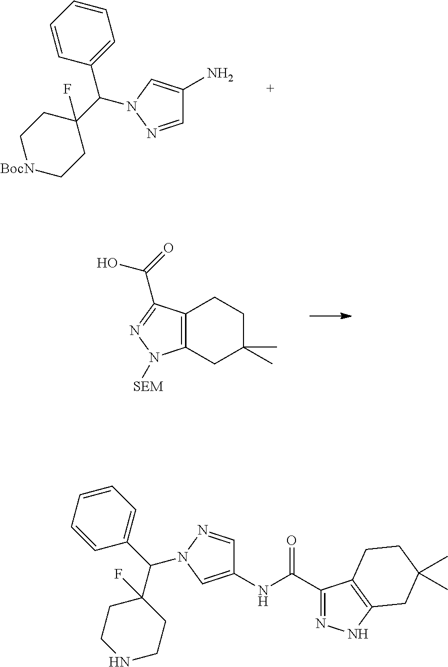 [SMILES: CC(C)(C)OC(=O)N1CCC(F)(C(c2ccccc2)n2cc(N)cn2)CC1 ; CC1(C)CCc2c(C(=O)O)nn(COCC[Si](C)(C)C)c2C1 ; CC1(C)CCc2c(C(=O)Nc3cnn(C(c4ccccc4)C4(F)CCNCC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with tert-butyl 4-((4-amino-1H-pyrazol-1-yl)(phenyl)methyl)-4-fluoropiperidine-1-carboxylate (Example A6).

SFC conditions: Lux Cellulose-4 (4.6×50 mm, 5 μm particle size) at 40% methanol w/0.1% NH₄OH; 5 mL/min, 120 bars, 40° C.

44a: ¹H NMR (400 MHz, DMSO-d₆) δ 13.02-12.58 (s, 1H), 10.19-9.91 (s, 1H), 7.70-7.65 (s, 1H), 7.63-7.61 (s, 1H), 7.61-7.59 (s, 1H), 7.42-7.30 (m, 3H), 5.71-5.46 (d, J=26.8 Hz, 1H), 2.88-2.77 (m, 2H), 2.72-2.63 (m, 4H), 2.41-2.37 (s, 2H), 1.76-1.35 (m, 6H), 0.99-0.93 (s, 6H); MS: m/z=451 (M+H); SFC retention time: 0.38 min.

44b: ¹H NMR (400 MHz, DMSO-d₆) δ 12.97-12.62 (s, 1H), 10.18-10.03 (s, 1H), 8.36-8.17 (m, 1H), 7.68-7.66 (s, 1H), 7.63-7.61 (s, 1H), 7.61-7.59 (s, 1H), 5.73-5.46 (d, J=26.9 Hz, 1H), 2.86-2.75 (m, 2H), 2.71-2.62 (m, 4H), 2.41-2.37 (s, 2H), 1.77-1.34 (m, 6H), 0.99-0.93 (s, 6H); MS: m/z=451 (M+H); SFC retention time: 0.64 min.

### Examples 45a and 45b

### 6,6-dimethyl-N-(1-((1-(methylsulfonyl)azetidin-3-yl)(phenyl)methyl)-1H-pyrazol-4-yl)-4,5,6,7-tetrahydro-1H-indazole-3-carboxamide

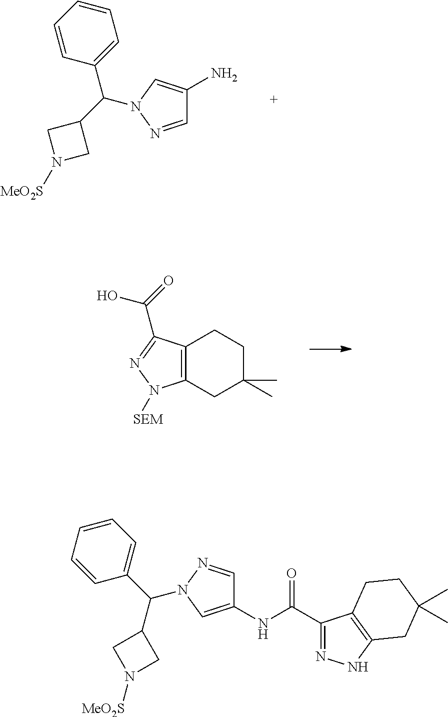 [SMILES: CS(=O)(=O)N1CC(C(c2ccccc2)n2cc(N)cn2)C1 ; CC1(C)CCc2c(C(=O)O)nn(COCC[Si](C)(C)C)c2C1 ; CC1(C)CCc2c(C(=O)Nc3cnn(C(c4ccccc4)C4CN(S(C)(=O)=O)C4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-((1-(methylsulfonyl)azetidin-3-yl)(phenyl)methyl)-1H-pyrazol-4-amine (Example A7).

SFC conditions: Lux Cellulose-4 (4.6×50 mm, 5 μm particle size) at 45% methanol w/0.1% NH₄OH; 5 mL/min, 120 bars, 40° C.

45a: ¹H NMR (400 MHz, DMSO-d₆) δ 8.59-8.56 (s, 1H), 8.18-8.14 (s, 1H), 7.53-7.44 (m, 3H), 7.42-7.36 (m, 2H), 5.73-5.69 (d, J=6.5 Hz, 1H), 4.87-4.78 (dd, J=12.0, 7.8 Hz, 1H), 4.50-4.40 (dd, J=12.0, 6.7 Hz, 1H), 2.84-2.80 (s, 3H), 2.70-2.62 (t, J=6.4 Hz, 2H), 2.37-2.31 (s, 2H), 1.48-1.38 (m, 2H), 0.97-0.91 (s, 6H); MS: m/z=483 (M+H); SFC retention time: 0.74 min.

45b: ¹H NMR (400 MHz, DMSO-d₆) δ 8.68-8.65 (s, 1H), 8.58-8.54 (s, 1H), 8.22-8.19 (s, 1H), 7.52-7.46 (m, 3H), 7.44-7.38 (m, 2H), 5.75-5.69 (d, J=6.7 Hz, 1H), 4.91-4.82 (dd, J=12.1, 7.8 Hz, 1H), 4.51-4.41 (dd, J=12.2, 6.9 Hz, 1H), 2.91-2.84 (s, 3H), 2.71-2.62 (t, J=6.1 Hz, 2H), 2.39-2.33 (s, 2H), 1.49-1.40 (t, J=6.4 Hz, 2H), 0.99-0.90 (s, 5H); MS: m/z=483 (M+H); SFC retention time: 1.74 min.

### Examples 46a and 46b

### 6,6-dimethyl-N-(1-(3-(methylsulfonyl)-1-phenylpropyl)-1H-pyrazol-4-yl)-4,5,6,7-tetrahydro-1H-indazole-3-carboxamide

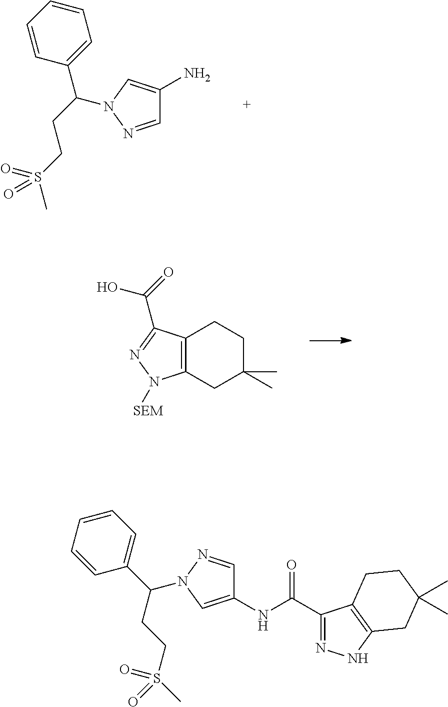 [SMILES: CS(=O)(=O)CCC(c1ccccc1)n1cc(N)cn1 ; CC1(C)CCc2c(C(=O)O)nn(COCC[Si](C)(C)C)c2C1 ; CC1(C)CCc2c(C(=O)Nc3cnn(C(CCS(C)(=O)=O)c4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(3-(methylsulfonyl)-1-phenylpropyl)-1H-pyrazol-4-amine (Example A53). Also, the stereoisomers were separated by preparative chiral HPLC instead of SFC.

Chiral HPLC Conditions: ChiralPak IB (4.6×250 mm, 5 μm particle size); eluent=hexane (0.1% Et₃N):EtOH 80:20; 1.0 ml/min, 5.0 MPA, 25° C.

46a: ¹H-NMR (CD₃OD, ppm) δ 8.08 (1H, s), 7.7 (1H, s), 7.37-7.26 (5H, m), 5.55-5.50 (1H, m), 3.29-2.83 (6H, m), 2.80-2.72 (2H, m), 2.67-2.59 (1H, m), 2.40 (2H, s), 1.55-1.50 (2H, t, J=6.3), 0.99 (6H, s); MS: m/z=456 (M+H); HPLC retention time: 16.70 min.

46b: ¹H-NMR (CD₃OD, ppm) δ 8.08 (1H, s), 7.7 (1H, s), 7.37-7.26 (5H, m), 5.55-5.50 (1H, m), 3.29-2.83 (6H, m), 2.80-2.72 (2H, m), 2.67-2.59 (1H, m), 2.40 (2H, s), 1.55-1.50 (2H, t, J=6.3), 0.99 (6H, s); MS: m/z=456 (M+H); HPLC retention time: 20.43 min.

### Examples 47a and 47b

### N-(1-(azetidin-3-yl(m-tolyl)methyl)-1H-pyrazol-4-yl)-6,6-dimethyl-4,5,6,7-tetrahydro-1H-indazole-3-carboxamide

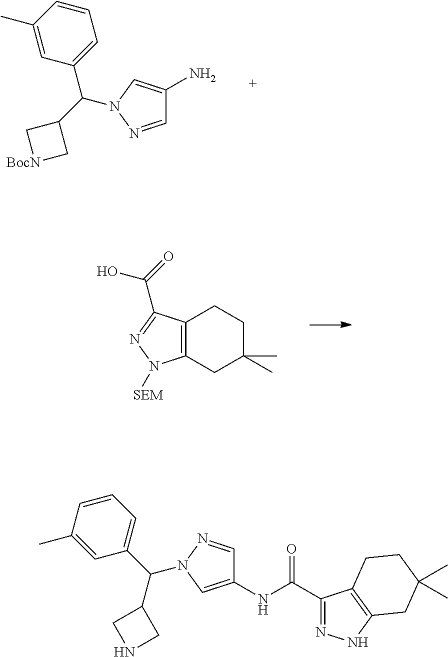

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with tert-butyl 3-((4-amino-1H-pyrazol-1-yl)(m-tolyl)methyl)azetidine-1-carboxylate (Example A54). Also, the stereoisomers were separated by preparative chiral HPLC instead of SFC.

Chiral HPLC Conditions: CHIRALPAK IC (4.6×150 mm, 5 μm particle size) at ACN:MeOH=50:50 (0.1% DEA) 40%; 3 ml/min, 100 bars, 35° C. 47a: ¹H-NMR (400 MHz, CD₃OD, ppm) δ 7.86 (s, 1H), 7.57 (s, 1H), 7.19-7.08 (m, 1H), 7.06-7.02 (m, 3H), 5.51-5.48 (d, J=8.8 Hz, 1H), 3.96-3.93 (m, 1H), 3.89-3.79 (m, 3H), 3.67-3.64 (m, 1H), 2.67-2.64 (t, J=12.4 Hz, 6.4 Hz), 2.32 (s, 2H), 2.22 (s, 3H), 1.47 (s, 2H), 0.92 (s, 9H); MS: m/z=419 (M+H); HPLC retention time: 10.33 min.

47b: ¹H-NMR (400 MHz, CD₃OD, ppm) δ 7.90 (s, 1H), 7.56 (s, 1H), 7.16-7.12 (m, 1H), 7.05-7.02 (m, 3H), 5.48-5.46 (d, J=8.8 Hz, 1H), 3.79-3.73 (m, 1H), 3.64-3.58 (m, 3H), 3.67-3.64 (m, 1H), 2.68-2.64 (t, J=12.4 Hz, 6.4 Hz), 2.32 (s, 2H), 2.23 (s, 3H), 1.47 (s, 2H), 0.92 (s, 9H); MS: m/z=419 (M+H); HPLC retention time: 5.46 min.

### Examples 48a and 48b

### N-(1-benzyl-1H-pyrazol-4-yl)-6-methoxy-6-methyl-4,5,6,7-tetrahydro-1H-indazole-3-carboxamide

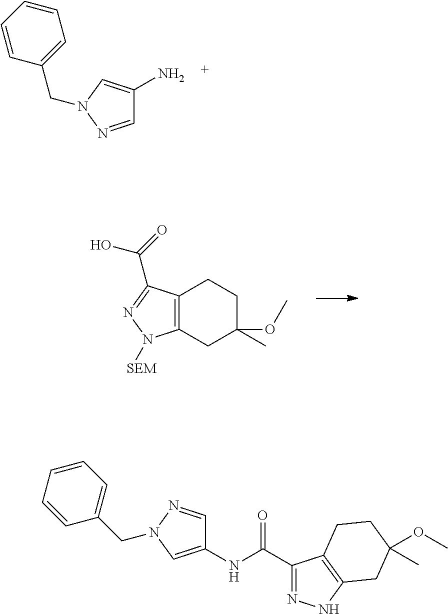 [SMILES: Nc1cnn(Cc2ccccc2)c1 ; COC1(C)CCc2c(C(=O)O)nn(COCC[Si](C)(C)C)c2C1 ; COC1(C)CCc2c(C(=O)Nc3cnn(Cc4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6-methoxy-6-methyl-1-((2-(trimethylsilyl)ethoxy)methyl)-4,5,6,7-tetrahydro-1H-indazole-3-carb oxylic acid (Example C20) and 3-((4-amino-1H-pyrazol-1-yl)methyl)benzonitrile (Example A1) with 1-benzyl-1H-pyrazol-4-amine (Example A2).

SFC conditions: Lux Cellulose-3 (4.6×50 mm, 5 μm particle size) at 20% methanol w/0.1% NH₄OH; 5 mL/min, 120 bars, 40° C.

48a: ¹H NMR (400 MHz, DMSO-d₆) δ 12.80 (s, 1H), 10.07 (s, 1H), 8.07 (s, 1H), 7.64 (s, 1H), 7.38-7.19 (m, 5H), 5.27 (s, 2H), 3.13 (s, 3H), 2.78-2.54 (m, 4H), 1.95-1.84 (m, 1H), 1.68-1.57 (m, 1H), 1.22 (s, 3H); MS: m/z=366 (M+H); SFC retention time: 0.38 min.

48b: ¹H NMR (400 MHz, DMSO-d₆) δ 12.80 (s, 1H), 10.07 (s, 1H), 8.07 (s, 1H), 7.64 (s, 1H), 7.38-7.20 (m, 5H), 5.27 (s, 2H), 3.13 (s, 3H), 2.77-2.55 (m, 4H), 1.95-1.85 (m, 1H), 1.68-1.57 (m, 1H), 1.22 (s, 3H); MS: m/z=366 (M+H); SFC retention time: 0.46 min.

### Examples 49a and 49b

### N-(1-((3-fluoroazetidin-3-yl)(phenyl)methyl)-1H-pyrazol-4-yl)-6,6-dimethyl-4,5,6,7-tetrahydro-1H-indazole-3-carboxamide

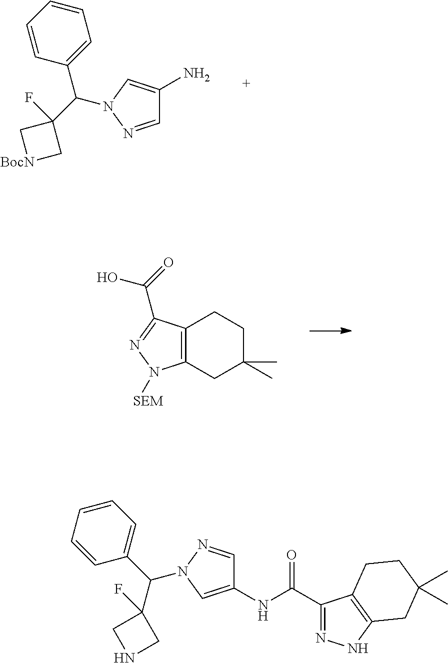 [SMILES: CC(C)(C)OC(=O)N1CC(F)(C(c2ccccc2)n2cc(N)cn2)C1 ; CC1(C)CCc2c(C(=O)O)nn(COCC[Si](C)(C)C)c2C1 ; CC1(C)CCc2c(C(=O)Nc3cnn(C(c4ccccc4)C4(F)CNC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with tert-butyl 3-((4-amino-1H-pyrazol-1-yl)(phenyl)methyl)-3-fluoroazetidine-1-carboxylate (Example A8).

SFC conditions: Chiralpak IC (4.6×50 mm, 5 μm particle size) at 35% methanol w/0.1% NH₄OH; 5 mL/min, 120 bars, 40° C.

49a: ¹H NMR (400 MHz, DMSO-d₆) δ 12.85-12.68 (s, 1H), 10.19-10.01 (s, 1H), 8.09-8.03 (s, 1H), 7.72-7.67 (s, 1H), 7.43-7.29 (m, 6H), 6.08-5.94 (d, J=28.8 Hz, 1H), 3.67-3.46 (m, 4H), 2.71-2.60 (t, J=6.4 Hz, 2H), 2.43-2.35 (s, 2H), 1.52-1.41 (t, J=6.4 Hz, 2H), 1.00-0.90 (s, 7H); MS: m/z=423 (M+H); SFC retention time: 0.51 min.

49b: ¹H NMR (400 MHz, DMSO-d₆) δ 12.91-12.62 (s, 1H), 10.13-10.09 (s, 1H), 8.07-8.04 (s, 1H), 7.72-7.68 (s, 1H), 7.44-7.31 (m, 6H), 6.06-5.94 (d, J=28.7 Hz, 1H), 3.68-3.48 (m, 4H), 2.70-2.61 (t, J=6.3 Hz, 2H), 2.41-2.36 (s, 2H), 1.52-1.42 (t, J=6.4 Hz, 2H); MS: m/z=423 (M+H); SFC retention time: 0.51 min.

### Examples 50a and 50b

### N-(1-benzyl-1H-pyrazol-4-yl)-1′,4,4′,5,5′,7′-hexahydro-3H-spiro[furan-2,6′-indazole]-3′-carboxamide

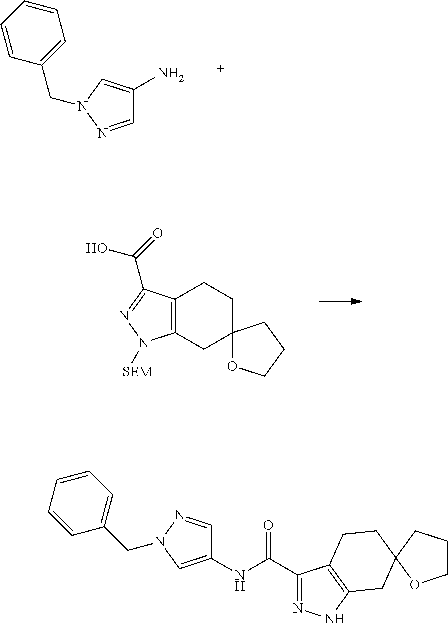 [SMILES: Nc1cnn(Cc2ccccc2)c1 ; C[Si](C)(C)CCOCn1nc(C(=O)O)c2c1CC1(CCCO1)CC2 ; O=C(Nc1cnn(Cc2ccccc2)c1)c1n[nH]c2c1CCC1(CCCO1)C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1′,4,4′,5,5′,7′-hexahydro-3H-spiro[furan-2,6′-indazole]-3′-ca rboxylic acid (Example C23) and 3-((4-amino-1H-pyrazol-1-yl)methyl)benzonitrile (Example A1) with 1-benzyl-1H-pyrazol-4-amine (Example A2).

SFC conditions: Lux Cellulose-1 (4.6×50 mm, 5 μm particle size) at 30% methanol w/0.1% NH₄OH; 5 mL/min, 120 bars, 40° C.

50a: ¹H NMR (400 MHz, DMSO-d₆) δ 12.81 (s, 1H), 10.08 (s, 1H), 8.07 (s, 1H), 7.64 (s, 1H), 7.37-7.20 (m, 5H), 5.27 (s, 2H), 3.75 (t, J=6.8 Hz, 2H), 2.77-2.58 (m, 4H), 1.98-1.86 (m, 2H), 1.84-1.58 (m, 4H); MS: m/z=378 (M+H); SFC retention time: 0.50 min.

50b: ¹H NMR (400 MHz, DMSO-d₆) δ 12.81 (s, 1H), 10.08 (s, 1H), 8.07 (s, 1H), 7.64 (s, 1H), 7.37-7.20 (m, 5H), 5.27 (s, 2H), 3.75 (t, J=6.8 Hz, 2H), 2.77-2.59 (m, 4H), 1.97-1.87 (m, 2H), 1.83-1.61 (m, 4H); MS: m/z=378 (M+H); SFC retention time: 0.91 min.

### Examples 51a and 51b

### N-(1-benzyl-1H-pyrazol-4-yl)-6-hydroxy-6-methyl-4,5,6,7-tetrahydro-1H-indazole-3-carboxamide

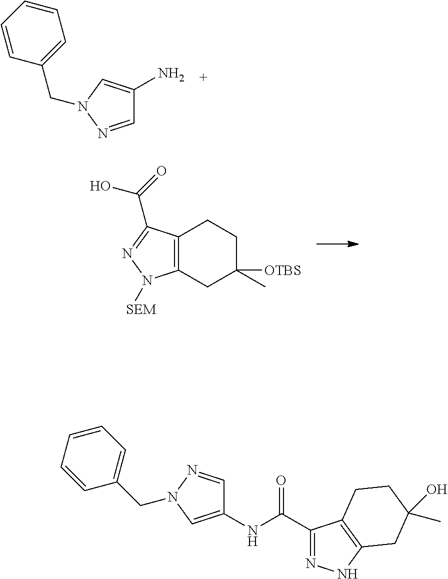 [SMILES: Nc1cnn(Cc2ccccc2)c1 ; CC1(O[Si](C)(C)C(C)(C)C)CCc2c(C(=O)O)nn(COCC[Si](C)(C)C)c2C1 ; CC1(O)CCc2c(C(=O)Nc3cnn(Cc4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6-((tert-butyldimethylsilyl)oxy)-6-methyl-1-((2-(trimethylsilyl)ethoxy)methyl)-4,5,6,7-tetrahydro-1H-indazole-3-carboxylic acid (Example C21) and 3-((4-amino-1H-pyrazol-1-yl)methyl)benzonitrile (Example A1) with 1-benzyl-1H-pyrazol-4-amine (Example A2).

SFC conditions: Lux Cellulose-1 (4.6×50 mm, 5 μm particle size) at 25% methanol w/0.1% NH₄OH; 5 mL/min, 120 bars, 40° C.

51a: ¹H NMR (400 MHz, DMSO-d₆) δ 12.75 (s, 1H), 10.06 (s, 1H), 8.07 (s, 1H), 7.64 (s, 1H), 7.37-7.20 (m, 5H), 5.27 (s, 2H), 4.49 (s, 1H), 2.79-2.60 (m, 2H), 2.59 (s, 2H), 1.75-1.66 (m, 1H), 1.62-1.52 (m, 1H), 1.21 (s, 3H); MS: m/z=352 (M+H); SFC retention time: 0.59 min.

51b: ¹H NMR (400 MHz, DMSO-d₆) δ 12.75 (s, 1H), 10.06 (s, 1H), 8.07 (s, 1H), 7.64 (s, 1H), 7.37-7.20 (m, 5H), 5.27 (s, 2H), 4.49 (s, 1H), 2.78-2.61 (m, 2H), 2.59 (s, 2H), 1.75-1.66 (m, 1H), 1.62-1.52 (m, 1H), 1.21 (s, 3H); MS: m/z=352 (M+H); SFC retention time: 0.70 min.

### Examples 52a-d

### N-(1-(3-(dimethylamino)-1-phenylpropyl)-1H-pyrazol-4-yl)-5a-methyl-1,4,4a,5,5a,6-hexahydrocyclopropa[f]indazole-3-carboxamide

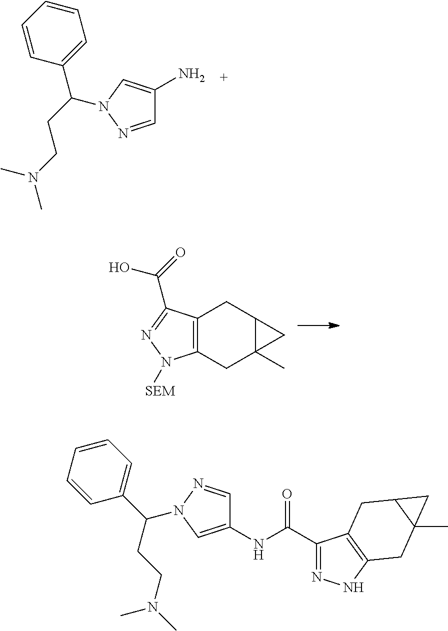 [SMILES: CN(C)CCC(c1ccccc1)n1cc(N)cn1 ; CC12Cc3c(c(C(=O)O)nn3COCC[Si](C)(C)C)CC1C2 ; CN(C)CCC(c1ccccc1)n1cc(NC(=O)c2n[nH]c3c2CC2CC2(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-H-indazole-3-carboxylic acid (Example C1) with 5a-methyl-1-((2-(trimethylsilyl)ethoxy)methyl)-1,4,4a,5,5a,6-hexahydrocyclopropa[f]indazole-3-carboxylic acid (Example C31) and 3-((4-amino-1H-pyrazol-1-yl)methyl)benzonitrile (Example A1) with 1-(3-(dimethylamino)-1-phenylpropyl)-1H-pyrazol-4-amine (Example A3). Also, the stereoisomers were separated by preparative chiral HPLC instead of SFC.

Chiral HPLC Conditions (52a/b): ChiralPak AD-H (4.6×250 mm, 5 μm particle size); eluent=hexane (0.1% Et₃N):EtOH 75:25; 1.0 ml/min, 7.3 MPA, 25° C.

52a: ¹H-NMR (300 MHz, CD₃OD, ppm) 8.05 (s, 1H), 7.65 (s, 1H), 7.25-7.32 (m, 5H), 5.35-5.40 (m, 1H), 2.89-3.05 (m, 2H), 2.54-2.70 (m, 2H), 2.31-2.37 (m, 3H), 2.15-2.27 (m, 7H), 1.32-1.37 (m, 1H), 1.22 (s, 3H), 1.05-1.06 (m, 1H), 1.11-1.12 (m, 1H); MS: m/z=419 (M+H); HPLC retention time: 12.35 min.

52b: ¹H-NMR (300 MHz, CD₃OD, ppm) 8.10 (s, 1H), 7.65 (s, 1H), 7.25-7.32 (m, 5H), 5.35-5.40 (m, 1H), 2.89-3.05 (m, 2H), 2.54-2.70 (m, 2H), 2.32-2.44 (m, 3H), 2.24-2.28 (m, 7H), 1.22-0.26 (m, 2H), 1.02-1.08 (m, 1H), 0.33-0.37 (m, 1H), 0.13-0.16 (m, 1H); MS: m/z=419 (M+H); HPLC retention time: 18.51 min.

Chiral HPLC Conditions (52c/d): ChiralPak IC (4.6×250 mm, 5 μm particle size); eluent=MTBE (0.1% Et₃N):IPA(0.4 IBA) 95:5; 0.5 ml/min, 6.6 MPA, 25° C.

52c: ¹H-NMR (300 MHz, CD₃OD, ppm) δ 8.05 (s, 1H), 7.65 (s, 1H), 7.25-7.32 (m, 5H), 5.34-5.40 (m, 1H), 2.89-3.00 (m, 2H), 2.55-2.70 (m, 2H), 2.31-2.41 (m, 2H), 2.21-2.29 (m, 7H), 1.22-1.26 (m, 4H), 1.02-1.08 (m, 1H), 0.33-0.37 (m, 1H), 0.13-0.16 (m, 1H); MS: m/z=419 (M+H); HPLC retention time: 12.96 min.

52d: ¹H-NMR (300 MHz, CD₃OD, ppm) δ 8.09 (s, 1H), 7.65 (s, 1H), 7.23-7.34 (m, 5H), 5.35-5.40 (m, 1H), 2.89-3.00 (m, 2H), 2.57-2.70 (m, 2H), 2.30-2.39 (m, 2H), 2.19-2.29 (m, 7H), 1.22-2.26 (m, 4H), 1.02-1.08 (m, 1H), 0.33-0.37 (m, 1H), 0.13-0.16 (m, 1H); MS: m/z=419 (M+H); HPLC retention time: 17.12 min.

### Examples 53a and 53b

### N-(1-(2-hydroxy-1-phenylethyl)-1H-pyrazol-4-yl)-6,6-dimethyl-4,5,6,7-tetrahydro-1H-indazole-3-carboxamide

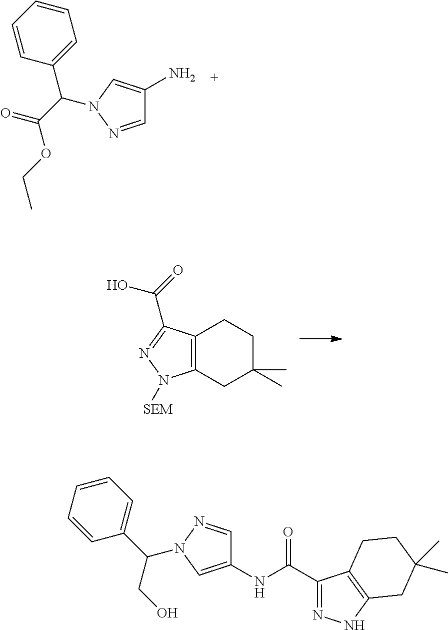 [SMILES: CCOC(=O)C(c1ccccc1)n1cc(N)cn1 ; CC1(C)CCc2c(C(=O)O)nn(COCC[Si](C)(C)C)c2C1 ; CC1(C)CCc2c(C(=O)Nc3cnn(C(CO)c4ccccc4)c3)n[nH]c2C1]

To a solution of ethyl 2-(4-aminopyrazol-1-yl)-2-phenyl-acetate (2.52 mmol, 618 mg, Example A11) and 6,6-dimethyl-1-(2-trimethylsilylethoxymethyl)-5,7-dihydro-4H-indazole-3-carboxylic acid (1.0 equiv., 2.52 mmol, 818 mg, Example C6) in dimethylformamide (10 mL) was added HATU (1.1 equiv., 2.77 mmol, 1050 mg) and N,N′-diisopropylethylamine (2.0 equiv., 5.04 mmol, 658 mg, 0.887 mL) and the mixture was stirred overnight at rt. The mixture was diluted with 100 mL EtOAc and extracted with 100 mL sat. NaHCO₃(aq) and 2×100 mL 1:1 H₂O:brine. The organic extracts were dried (Na₂SO₄) and concentrated in vacuo. Purification by CombiFlash (40 g; dry load; 100:0 to 50:50 heptane:EtOAc over 20 minutes) provided ethyl 2-[4-[[6,6-dimethyl-1-(2-trimethylsilylethoxymethyl)-5,7-dihydro-4H-indazole-3-carbonyl]amino]pyrazol-1-yl]-2-phenyl-acetate (611 mg, 1.107 mmol, 43.9% yield).

A solution of ethyl 2-[4-[[6,6-dimethyl-1-(2-trimethylsilylethoxymethyl)-5,7-dihydro-4H-indazole-3-carbonyl]amino]pyrazol-1-yl]-2-phenyl-acetate (200 mg, 0.3625 mmol) in tetrahydrofuran (2 mL) was cooled to 0° C., then lithium aluminum hydride (2.0 mol/L) in THF (2 equiv., 0.7249 mmol, 330 mg, 0.36 mL) was added dropwise. The mixture was stirred for 30 minutes at rt. EtOAc (˜1 mL) was added to quench excess hydride, then ˜5 mL sat. Rochelle's salt was added and the mixture was stirred vigorously overnight. The mixture was diluted with 50 mL EtOAc and washed with 50 mL brine. The organic extracts were dried (Na₂SO₄) and concentrated in vacuo. Purification by CombiFlash (12 g; dry load; 50:50 to 0:100 heptane:EtOAc over 16 minutes) provided N-[1-(2-hydroxy-1-phenyl-ethyl)pyrazol-4-yl]-6,6-dimethyl-1-(2-trimethylsilylethoxymethyl)-5,7-dihydro-4H-indazole-3-carboxamide (112 mg, 0.2197 mmol, 60.63% yield).

To a solution of N-[1-(2-hydroxy-1-phenyl-ethyl)pyrazol-4-yl]-6,6-dimethyl-1-(2-trimethylsilylethoxymethyl)-5,7-dihydro-4H-indazole-3-carboxamide (112 mg, 0.2197 mmol) in trifluoroacetic acid (2 mL) was added triisopropylsilane (5 equiv., 1.099 mmol, 175.7 mg, 0.227 mL) and a few drops of CH₂Cl₂ to homogenize and the mixture was stirred for 3 hours at rt. After in vacuo concentration, the residue was purified by reverse phase HPLC, then SFC with a chiral stationary phase to provide the title compounds as single enantiomers.

SFC conditions: Lux Cellulose-3 (4.6×50 mm, 5 μm particle size) at 20% methanol w/0.1% NH₄OH; 5 mL/min, 120 bars, 40° C.

53a: ¹H NMR (400 MHz, DMSO-d₆) δ 12.89-12.66 (s, 1H), 10.19-9.93 (s, 1H), 8.15-8.12 (s, 1H), 7.68-7.64 (s, 1H), 7.36-7.23 (m, 5H), 5.42-5.35 (dd, J=8.3, 5.2 Hz, 1H), 5.08-5.02 (t, J=5.4 Hz, 1H), 4.24-4.13 (ddd, J=11.4, 8.5, 5.8 Hz, 1H), 3.98-3.89 (m, 1H), 2.71-2.62 (t, J=6.4 Hz, 2H), 2.41-2.36 (s, 2H), 1.52-1.42 (t, J=6.4 Hz, 2H), 0.99-0.93 (s, 6H); MS: m/z=380 (M+H); SFC retention time: 0.64 min.

53b: ¹H NMR (400 MHz, DMSO-d₆) δ 12.89-12.66 (s, 1H), 10.19-9.93 (s, 1H), 8.15-8.12 (s, 1H), 7.68-7.64 (s, 1H), 7.36-7.23 (m, 5H), 5.42-5.35 (dd, J=8.3, 5.2 Hz, 1H), 5.08-5.02 (t, J=5.4 Hz, 1H), 4.24-4.13 (ddd, J=11.4, 8.5, 5.8 Hz, 1H), 3.98-3.89 (m, 1H), 2.71-2.62 (t, J=6.4 Hz, 2H), 2.41-2.36 (s, 2H), 1.52-1.42 (t, J=6.4 Hz, 2H), 0.99-0.93 (s, 6H); MS: m/z=380 (M+H); SFC retention time: 0.73 min.

### Examples 54a and 54b

### 6,6-dimethyl-N-(1-(1-(pyridin-3-yl)propyl)-1H-pyrazol-4-yl)-4,5,6,7-tetrahydro-1H-indazole-3-carboxamide

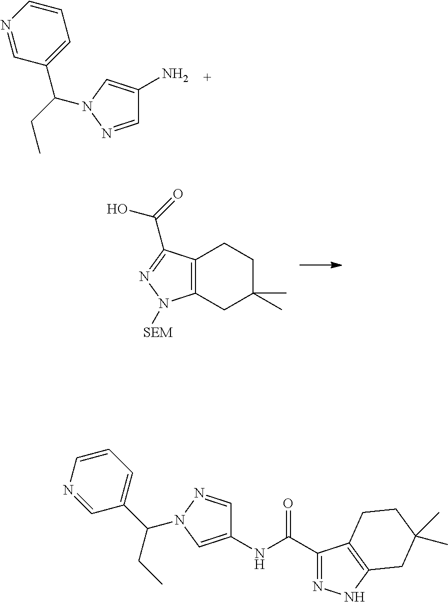 [SMILES: CCC(c1cccnc1)n1cc(N)cn1 ; CC1(C)CCc2c(C(=O)O)nn(COCC[Si](C)(C)C)c2C1 ; CCC(c1cccnc1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(pyridin-3-yl)propyl)-1H-pyrazol-4-amine (Example A10).

SFC conditions: Lux Cellulose-1 (4.6×50 mm, 5 μm particle size) at 30% isopropanol w/0.1% NH₄OH; 5 mL/min, 120 bars, 40° C.

54a: ¹H NMR (400 MHz, DMSO-d₆) δ 12.91-12.62 (s, 1H), 10.16-9.87 (s, 1H), 8.57-8.54 (d, J=2.2 Hz, 1H), 8.50-8.46 (dd, J=4.8, 1.6 Hz, 1H), 8.18-8.14 (s, 1H), 7.77-7.71 (dt, J=8.0, 2.0 Hz, 1H), 7.70-7.66 (s, 1H), 7.39-7.32 (dd, J=7.9, 4.8 Hz, 1H), 5.46-5.19 (dd, J=9.4, 6.1 Hz, 1H), 2.71-2.63 (t, J=6.2 Hz, 2H), 2.41-2.28 (m, 3H), 2.16-2.04 (m, 1H), 1.51-1.42 (t, J=6.4 Hz, 2H), 0.99-0.93 (s, 6H), 0.86-0.78 (t, J=7.2 Hz, 3H); MS: m/z=379 (M+H); SFC retention time: 0.56 min.

54b: ¹H NMR (400 MHz, DMSO-d₆) δ 12.89-12.58 (s, 1H), 10.18-9.91 (s, 1H), 8.58-8.53 (s, 1H), 8.52-8.45 (d, J=4.5 Hz, 1H), 8.18-8.14 (s, 1H), 7.78-7.71 (dt, J=7.9, 2.0 Hz, 1H), 7.69-7.66 (s, 1H), 7.40-7.31 (dd, J=7.9, 4.8 Hz, 1H), 5.46-5.23 (dd, J=9.4, 6.1 Hz, 1H), 2.71-2.61 (t, J=6.2 Hz, 2H), 2.41-2.28 (m, 3H), 2.18-2.04 (m, 1H), 1.51-1.43 (t, J=6.4 Hz, 2H), 0.99-0.92 (s, 6H), 0.86-0.77 (t, J=7.2 Hz, 3H); MS: m/z=379 (M+H); SFC retention time: 0.68 min.

### Examples 55a and 55b

### N-(1-(2-hydroxy-2-methyl-1-phenylpropyl)-1H-pyrazol-4-yl)-6,6-dimethyl-4,5,6,7-tetrahydro-1H-indazole-3-carboxamide

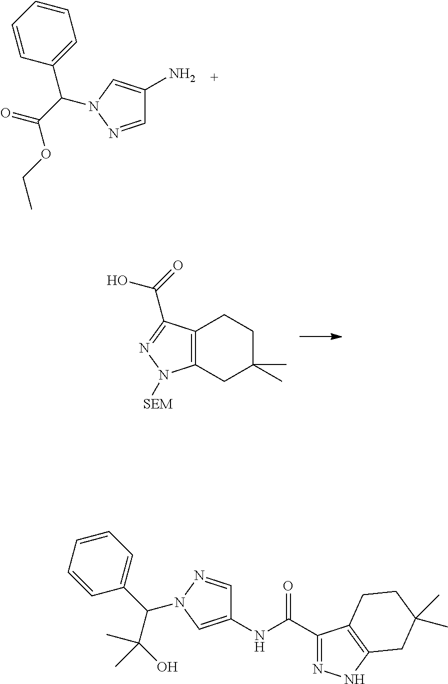 [SMILES: CCOC(=O)C(c1ccccc1)n1cc(N)cn1 ; CC1(C)CCc2c(C(=O)O)nn(COCC[Si](C)(C)C)c2C1 ; CC1(C)CCc2c(C(=O)Nc3cnn(C(c4ccccc4)C(C)(C)O)c3)n[nH]c2C1]

Prepared in an analogous manner to N-(1-(2-hydroxy-1-phenylethyl)-1H-pyrazol-4-yl)-6,6-dimethyl-4,5,6,7-tetrahydro-1H-indazole-3-carboxamide (Examples 53a and 53b), replacing the lithium aluminum hydride solution with 5.0 equiv. of methylmagnesium bromide solution (3.0 M in Et₂O).

SFC conditions: Chiralpak IC (4.6×50 mm, 5 μm particle size) at 30% methanol w/0.10% NH₄OH; 5 mL/min, 120 bars, 40° C.

55a: ¹H NMR (400 MHz, DMSO-d₆) δ 12.93-12.56 (s, 1H), 10.14-9.97 (s, 1H), 8.36-8.32 (s, 1H), 7.69-7.64 (s, 1H), 7.60-7.53 (m, 2H), 7.35-7.24 (m, 3H), 5.28-5.18 (s, 1H), 5.08-4.96 (s, 1H), 2.72-2.60 (t, J=6.1 Hz, 2H), 2.42-2.35 (s, 2H), 1.52-1.39 (t, J=6.4 Hz, 2H), 1.14-1.08 (s, 3H), 1.09-1.02 (s, 3H), 1.01-0.87 (s, 6H). MS: m/z=408 (M+H); SFC retention time: 0.38 min.

55b: ¹H NMR (400 MHz, DMSO-d₆) δ 12.85-12.64 (s, 1H), 10.16-9.97 (s, 1H), 8.36-8.31 (s, 1H), 7.68-7.64 (s, 1H), 7.61-7.54 (m, 2H), 7.35-7.22 (m, 3H), 5.35-5.16 (s, 1H), 5.08-4.89 (s, 1H), 2.72-2.62 (t, J=6.4 Hz, 2H), 2.41-2.36 (s, 2H), 1.51-1.43 (t, J=6.4 Hz, 2H), 1.14-1.08 (s, 3H), 1.07-1.03 (s, 3H), 1.00-0.93 (s, 6H); MS: m/z=408 (M+H); SFC retention time: 0.43 min.

### Examples 56a and 56b

### N-(1-benzyl-1H-pyrazol-4-yl)-6-ethyl-6-methyl-4,5,6,7-tetrahydro-1H-indazole-3-carboxamide

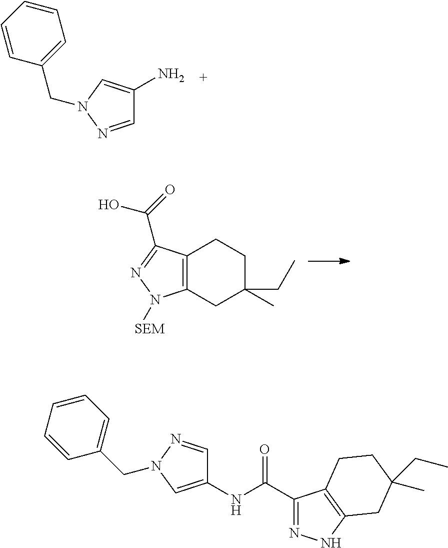 [SMILES: Nc1cnn(Cc2ccccc2)c1 ; CCC1(C)CCc2c(C(=O)O)nn(COCC[Si](C)(C)C)c2C1 ; CCC1(C)CCc2c(C(=O)Nc3cnn(Cc4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6-ethyl-6-methyl-1-((2-(trimethylsilyl)ethoxy)methyl)-4,5,6,7-tetrahydro-1H-indazole-3-carboxylic acid (Example C22) and 3-((4-amino-1H-pyrazol-1-yl)methyl)benzonitrile (Example A1) with 1-benzyl-1H-pyrazol-4-amine (Example A2).

SFC conditions: Chiralpak IA (4.6×50 mm, 5 μm particle size) at 45% methanol w/0.1% NH₄OH; 5 mL/min, 120 bars, 40° C.

56a: ¹H NMR (400 MHz, DMSO-d₆) δ 12.78 (s, 1H), 10.07 (s, 1H), 8.07 (s, 1H), 7.64 (s, 1H), 7.38-7.26 (m, 3H), 7.26-7.20 (m, 2H), 5.27 (s, 2H), 2.76-2.54 (m, 2H), 2.45-2.29 (m, 2H), 1.48 (t, J=6.3 Hz, 2H), 1.39-1.21 (m, 2H), 0.91-0.80 (m, 6H); MS: m/z=364 (M+H); SFC retention time: 0.59 min.

56b: ¹H NMR (400 MHz, DMSO-d₆) δ 12.77 (s, 1H), 10.07 (s, 1H), 8.07 (s, 1H), 7.64 (s, 1H), 7.37-7.26 (m, 3H), 7.25-7.20 (m, 2H), 5.27 (s, 2H), 2.74-2.55 (m, 2H), 2.44-2.29 (m, 2H), 1.48 (t, J=6.3 Hz, 2H), 1.37-1.22 (m, 2H), 0.91-0.81 (m, 6H); MS: m/z=364 (M+H); SFC retention time: 0.67 min.

### Examples 57a and 57b

### N-(1-(2-(azetidin-1-yl)-1-phenylethyl)-1H-pyrazol-4-yl)-6,6-dimethyl-4,5,6,7-tetrahydro-1H-indazole-3-carboxamide

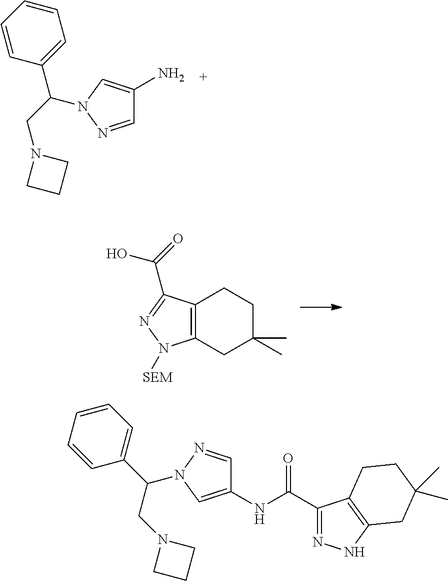

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(2-(azetidin-1-yl)-1-phenylethyl)-1H-pyrazol-4-amine (Example A12).

SFC conditions: Lux Cellulose-4 (4.6×50 mm, 5 μm particle size) at 40% methanol w/0.1% NH₄OH; 5 mL/min, 120 bars, 40° C.

57a: ¹H NMR (400 MHz, DMSO-d₆) δ 12.91-12.60 (s, 1H), 10.12-9.89 (s, 1H), 8.12-8.08 (s, 1H), 7.66-7.60 (s, 1H), 7.36-7.21 (m, 5H), 5.33-5.17 (dd, J=8.7, 5.7 Hz, 1H), 3.28-3.25 (m, 1H), 3.13-2.89 (m, 5H), 2.71-2.62 (t, J=6.3 Hz, 2H), 2.41-2.36 (s, 2H), 1.93-1.80 (p, J=6.9 Hz, 2H), 1.51-1.43 (t, J=6.4 Hz, 2H), 1.01-0.90 (s, 6H); MS: m/z=419 (M+H); SFC retention time: 0.46 min.

57b: ¹H NMR (400 MHz, DMSO-d₆) δ 12.93-12.58 (s, 1H), 10.12-9.87 (s, 1H), 8.21-7.98 (s, 1H), 7.69-7.49 (s, 1H), 7.43-7.07 (m, 5H), 5.37-5.16 (dd, J=8.7, 5.7 Hz, 1H), 3.30-3.25 (m, 1H), 3.12-2.90 (m, 5H), 2.70-2.62 (t, J=6.3 Hz, 2H), 2.42-2.36 (s, 2H), 1.92-1.81 (p, J=6.9 Hz, 2H), 1.51-1.41 (t, J=6.3 Hz, 2H); MS: m/z=419 (M+H); SFC retention time: 0.54 min.

### Examples 58a and 58b

### 6,6-dimethyl-N-(1-(3-(methyl(oxetan-3-yl)amino)-1-phenylpropyl)-1H-pyrazol-4-yl)-4,5,6,7-tetrahydro-1H-indazole-3-carboxamide

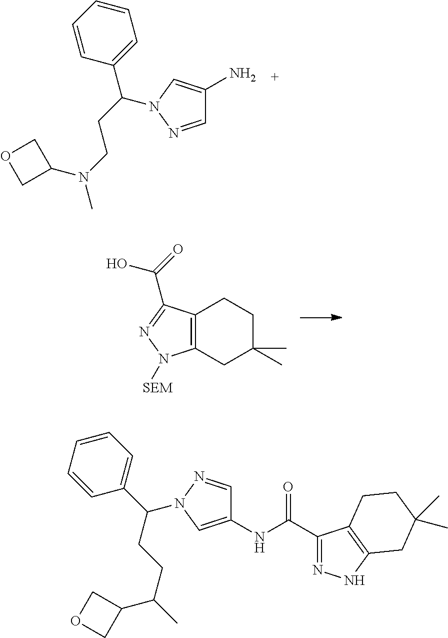 [SMILES: CN(CCC(c1ccccc1)n1cc(N)cn1)C1COC1 ; CC1(C)CCc2c(C(=O)O)nn(COCC[Si](C)(C)C)c2C1 ; CC(CCC(c1ccccc1)n1cc(NC(=O)c2n[nH]c3c2CCC(C)(C)C3)cn1)C1COC1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(3-(methyl(oxetan-3-yl)amino)-1-phenylpropyl)-1H-pyrazol-4-amine (Example A28).

SFC conditions: (S,S)-Whelk-O1 (4.6×50 mm, 5 μm particle size) at 40% methanol w/0.1% NH₄OH; 5 mL/min, 120 bars, 40° C.

58a: ¹H NMR (400 MHz, DMSO-d₆) δ 12.78 (s, 1H), 10.05 (s, 1H), 8.10 (s, 1H), 7.66 (s, 1H), 7.39-7.23 (m, 5H), 5.45 (dd, J=9.5, 5.4 Hz, 1H), 4.41 (q, J=6.9 Hz, 2H), 4.25 (dt, J=9.6, 6.1 Hz, 2H), 3.42 (p, J=6.5 Hz, 1H), 2.66 (t, J=6.1 Hz, 2H), 2.38 (s, 2H), 2.25-2.08 (m, 2H), 2.05 (s, 3H), 1.99 (dt, J=12.7, 6.0 Hz, 1H), 1.47 (t, J=6.4 Hz, 2H), 0.96 (s, 6H); MS: m/z=463 (M+H); SFC retention time: 0.71 min.

58b: ¹H NMR (400 MHz, DMSO-d₆) δ 12.78 (s, 1H), 10.05 (s, 1H), 8.10 (s, 1H), 7.66 (s, 1H), 7.52-7.09 (m, 5H), 5.45 (dd, J=9.5, 5.4 Hz, 1H), 4.41 (q, J=7.0 Hz, 2H), 4.25 (dt, J=9.7, 6.1 Hz, 2H), 3.42 (p, J=6.5 Hz, 1H), 2.66 (t, J=6.2 Hz, 2H), 2.38 (s, 2H), 2.29-2.07 (m, 2H), 2.05 (s, 3H), 2.03-1.93 (m, 1H), 1.47 (t, J=6.3 Hz, 2H), 0.96 (s, 6H); MS: m/z=463 (M+H); SFC retention time: 0.78 min.

### Example 59

### N-(1-(3-(dimethylamino)-1-phenylpropyl)-1H-pyrazol-4-yl)-1′,4,4′,5,5′,7′-hexahydro-3H-spiro[furan-2,6′-indazole]-3′-carboxamide

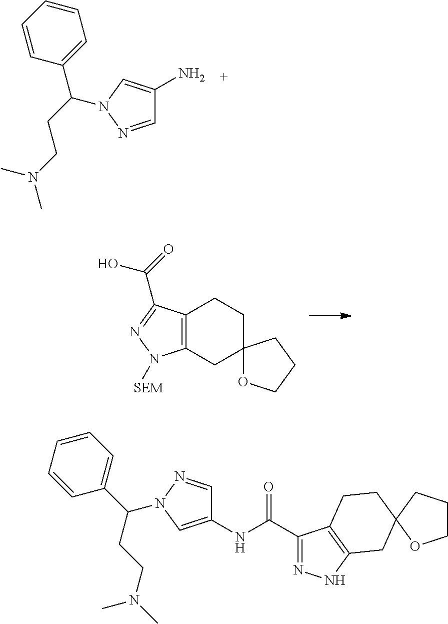 [SMILES: CN(C)CCC(c1ccccc1)n1cc(N)cn1 ; C[Si](C)(C)CCOCn1nc(C(=O)O)c2c1CC1(CCCO1)CC2 ; CN(C)CCC(c1ccccc1)n1cc(NC(=O)c2n[nH]c3c2CCC2(CCCO2)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1′,4,4′,5,5′,7′-hexahydro-3H-spiro[furan-2,6′-indazole]-3′-carboxylic acid (Example C23) and 3-((4-amino-1H-pyrazol-1-yl)methyl)benzonitrile (Example A1) with 1-(3-(dimethylamino)-1-phenylpropyl)-1H-pyrazol-4-amine (Example A3).

59: ¹H NMR (400 MHz, DMSO-d₆) δ 12.81 (s, 1H), 10.06 (s, 1H), 8.08 (s, 1H), 7.66 (s, 1H), 7.37-7.22 (m, 5H), 5.47-5.36 (m, 1H), 3.75 (t, J=6.8 Hz, 2H), 2.76-1.60 (m, 20H); MS: m/z=449 (M+H).

### Examples 60a and 60b

### N-(1-((1,1-dioxidotetrahydro-2H-thiopyran-4-yl)(phenyl)methyl)-1H-pyrazol-4-yl)-6,6-dimethyl-4,5,6,7-tetrahydro-1H-indazole-3-carboxamide

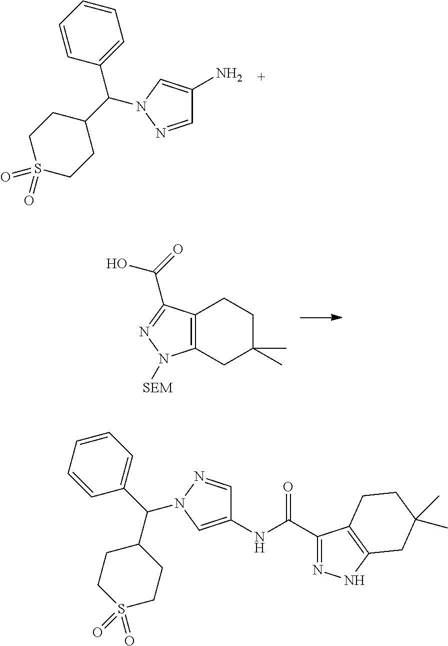 [SMILES: Nc1cnn(C(c2ccccc2)C2CCS(=O)(=O)CC2)c1 ; CC1(C)CCc2c(C(=O)O)nn(COCC[Si](C)(C)C)c2C1 ; CC1(C)CCc2c(C(=O)Nc3cnn(C(c4ccccc4)C4CCS(=O)(=O)CC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 4-((4-amino-1H-pyrazol-1-yl)(phenyl)methyl)tetrahydro-2H-thiopyran 1,1-dioxide (Example A55). Also, the stereoisomers were separated by preparative chiral HPLC instead of SFC.

Chiral HPLC Conditions ChiralPak IB-3 (4.6×50 mm, 3 μm particle size); eluent=Hex:EtOH 60:40; 1.0 ml/min, 3.5 MPA, 25° C.:

60a: ¹H-NMR (CDCl₃, 300 MHz): δ 8.62 (s, 1H), 8.14 (s, 1H), 7.56 (s, 1H), 7.43-7.30 (m, 5H), 4.83 (d, J=10.5 Hz, 2H), 3.03-2.91 (m, 4H), 2.86-2.73 (m, 3H), 2.41 (s, 2H), 1.95-1.79 (m, 4H), 1.56 (t, J=6.3 Hz, 2H), 1.01 (s, 6H); MS: m/z=482 (M+H); HPLC retention time: 1.97 min.

60b: ¹H-NMR (CDCl₃, 300 MHz): δ 8.60 (s, 1H), 8.14 (s, 1H), 7.56 (s, 1H), 7.43-7.29 (m, 5H), 4.84 (d, J=10.8 Hz, 2H), 3.04-2.91 (m, 4H), 2.86-2.73 (m, 3H), 2.41 (s, 2H), 1.95-1.79 (m, 4H), 1.56 (t, J=6.3 Hz, 2H), 1.01 (s, 6H); MS: m/z=482 (M+H); HPLC retention time: 3.04 min.

### Examples 61a and 61b

### 6,6-dimethyl-N-(1-(2-(N-methylmethylsulfonamido)-1-phenylethyl)-1H-pyrazol-4-yl)-4,5,6,7-tetrahydro-1H-indazole-3-carboxamide

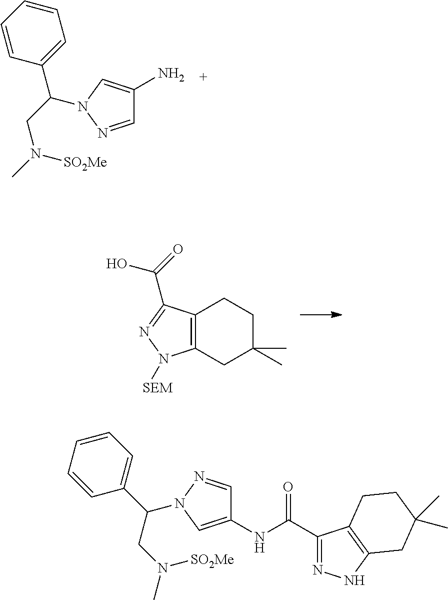 [SMILES: CN(CC(c1ccccc1)n1cc(N)cn1)S(C)(=O)=O ; CC1(C)CCc2c(C(=O)O)nn(COCC[Si](C)(C)C)c2C1 ; CN(CC(c1ccccc1)n1cc(NC(=O)c2n[nH]c3c2CCC(C)(C)C3)cn1)S(C)(=O)=O]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with N-(2-(4-amino-1H-pyrazol-1-yl)-2-phenylethyl)-N-methylmethanesulfonamide (Example A13).

SFC conditions: Chiralpak AD (4.6×50 mm, 5 μm particle size) at 45% methanol w/0.1% NH₄OH; 5 mL/min, 120 bars, 40° C.

61a: ¹H NMR (400 MHz, DMSO-d₆) δ 12.89-12.68 (s, 1H), 10.23-9.95 (s, 1H), 8.23-8.18 (s, 1H), 7.75-7.70 (s, 1H), 7.44-7.25 (m, 5H), 5.69-5.61 (dd, J=9.2, 5.5 Hz, 1H), 4.03-3.93 (dd, J=14.2, 9.3 Hz, 1H), 3.87-3.75 (dd, J=14.3, 5.5 Hz, 1H), 2.85-2.77 (s, 3H), 2.71-2.61 (m, 5H), 2.42-2.36 (s, 2H), 1.52-1.41 (t, J=6.3 Hz, 2H), 1.02-0.91 (s, 6H); MS: m/z=471 (M+H); SFC retention time: 0.59 min.

61b: ¹H NMR (400 MHz, DMSO-d₆) δ 12.87-12.70 (s, 1H), 10.23-9.93 (s, 1H), 8.23-8.18 (s, 1H), 7.74-7.71 (s, 1H), 7.44-7.26 (m, 5H), 5.78-5.52 (dd, J=9.0, 5.7 Hz, 1H), 4.07-3.90 (dd, J=14.3, 9.3 Hz, 1H), 3.88-3.68 (dd, J=14.3, 5.4 Hz, 1H), 2.83-2.78 (s, 3H), 2.71-2.61 (m, 5H), 2.41-2.36 (s, 2H), 1.50-1.41 (t, J=6.5 Hz, 2H), 0.98-0.90 (s, 7H); MS: m/z=471 (M+H); SFC retention time: 0.59 min.

### Examples 62a and 62b

### 6,6-dimethyl-N-(1-(2-morpholino-1-phenylethyl)-1H-pyrazol-4-yl)-4,5,6,7-tetrahydro-1H-indazole-3-carboxamide

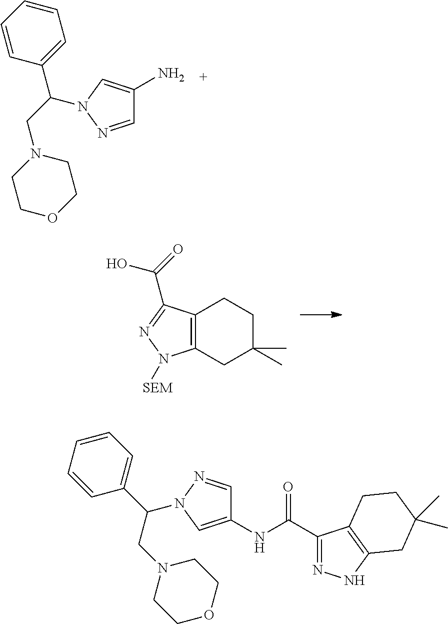 [SMILES: Nc1cnn(C(CN2CCOCC2)c2ccccc2)c1 ; CC1(C)CCc2c(C(=O)O)nn(COCC[Si](C)(C)C)c2C1 ; CC1(C)CCc2c(C(=O)Nc3cnn(C(CN4CCOCC4)c4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(2-morpholino-1-phenylethyl)-1H-pyrazol-4-amine (Example A29).

SFC conditions: Lux Cellulose-4 (4.6×50 mm, 5 μm particle size) at 30% methanol w/0.1% NH₄OH; 5 mL/min, 120 bars, 40° C.

62a: ¹H NMR (400 MHz, DMSO-d₆) δ 12.78 (s, 1H), 10.06 (s, 1H), 8.15 (s, 1H), 7.65 (s, 1H), 7.46-7.18 (m, 5H), 5.63 (dd, J=9.0, 5.3 Hz, 1H), 3.48 (t, J=4.7 Hz, 5H), 2.87 (dd, J=13.2, 5.3 Hz, 2H), 2.66 (t, J=6.3 Hz, 4H), 2.43-2.28 (m, 5H), 1.47 (t, J=6.4 Hz, 2H), 0.96 (s, 6H); MS: m/z=449 (M+H); SFC retention time: 0.68 min.

62b: ¹H NMR (400 MHz, DMSO-d₆) δ 12.82 (s, 1H), 10.12 (s, 1H), 8.17 (s, 1H), 7.64 (s, 1H), 7.43-7.14 (m, 5H), 5.64 (dd, J=9.0, 5.2 Hz, 1H), 3.48 (t, J=4.7 Hz, 4H), 2.86 (dd, J=13.2, 5.2 Hz, 1H), 2.66 (t, J=6.2 Hz, 2H), 2.45-2.28 (m, 4H), 1.47 (t, J=6.4 Hz, 2H), 0.96 (s, 6H); MS: m/z=449 (M+H); SFC retention time: 0.57 min.

### Examples 63a and 63b

### N-(1-((1-(2-fluoroethyl)piperidin-4-yl)(phenyl)methyl)-1H-pyrazol-4-yl)-6,6-dimethyl-4,5,6,7-tetrahydro-1H-indazole-3-carboxamide

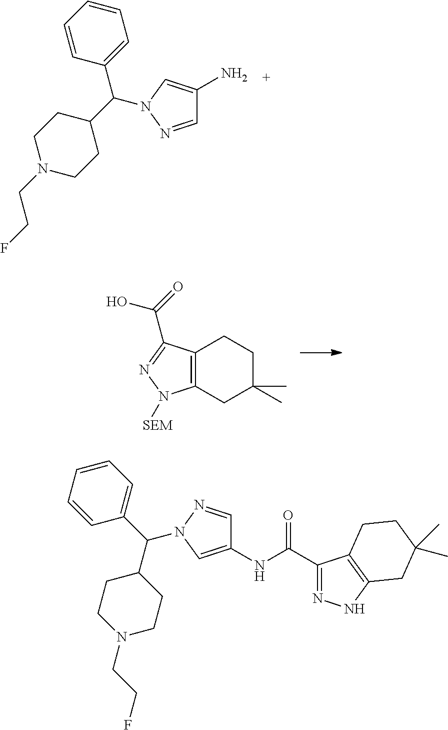 [SMILES: Nc1cnn(C(c2ccccc2)C2CCN(CCF)CC2)c1 ; CC1(C)CCc2c(C(=O)O)nn(COCC[Si](C)(C)C)c2C1 ; CC1(C)CCc2c(C(=O)Nc3cnn(C(c4ccccc4)C4CCN(CCF)CC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-((1-(2-fluoroethyl)piperidin-4-yl)(phenyl)methyl)-1H-pyrazol-4-amine (Example A57). Also, the stereoisomers were separated by preparative chiral HPLC instead of SFC.

Chiral HPLC Conditions: ChiralPak IA-3 (4.6×50 mm, 3 μm particle size); eluent=Hex (0.1% Et₃N):IPA 70:30; 1.0 ml/min, 4.0 MPA, 25° C.

63a: ¹H-NMR (300 MHz, CD₃OD, ppm) δ 8.15 (s, 1H), 7.68 (s, 1H), 7.50-7.53 (m, 2H), 7.28-7.39 (m, 3H), 4.88-4.98 (m, 1H), 4.64-4.67 (m, 1H), 4.48-4.51 (m, 1H), 2.96-3.09 (m, 2H), 2.77-2.81 (m, 3H), 2.66-2.69 (m, 1H), 2.51-2.54 (m, 1H), 2.44-2.48 (m, 2H), 2.09-2.17 (m, 2H), 1.56-1.60 (m, 2H), 1.36-1.50 (m, 5H), 1.01-1.20 (s, 6H); MS: m/z=479 (M+H); HPLC retention time: 3.62 min.

63b: ¹H-NMR (300 MHz, CD₃OD, ppm) δ 8.15 (s, 1H), 7.68 (s, 1H), 7.50-7.53 (m, 2H), 7.28-7.39 (m, 3H), 4.89-4.98 (m, 1H), 4.65-4.68 (m, 1H), 4.49-4.52 (m, 1H), 2.98-3.09 (m, 3H), 2.70-2.81 (m, 4H), 2.44-2.55 (m, 3H), 2.12-2.20 (m, 2H), 1.55-1.60 (m, 2H), 1.42-1.47 (m, 5H), 1.05 (s, 6H); MS: m/z=479 (M+H); HPLC retention time: 5.64 min.

### Examples 64a and 64b

### 6,6-dimethyl-N-(1-(2-(methylsulfonyl)-1-phenylethyl)-1H-pyrazol-4-yl)-4,5,6,7-tetrahydro-1H-indazole-3-carboxamide

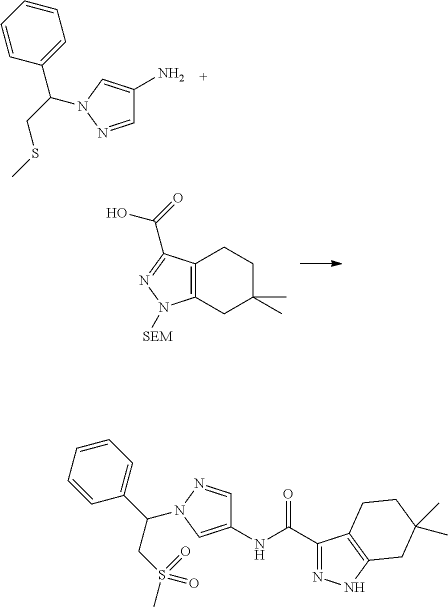 [SMILES: CSCC(c1ccccc1)n1cc(N)cn1 ; CC1(C)CCc2c(C(=O)O)nn(COCC[Si](C)(C)C)c2C1 ; CC1(C)CCc2c(C(=O)Nc3cnn(C(CS(C)(=O)=O)c4ccccc4)c3)n[nH]c2C1]

To a solution of 1-(2-methylsulfanyl-1-phenyl-ethyl)pyrazol-4-amine (2.53 mmol, 590 mg, Example A14) and 6,6-dimethyl-1-(2-trimethylsilylethoxymethyl)-5,7-dihydro-4H-indazole-3-carboxylic acid (1.0 equiv., 2.53 mmol, 821 mg, Example C6) in dimethylformamide (10 mL) was added HATU (1.0 equiv., 2.53 mmol, 992 mg) and N,N′-diisopropylethylamine (1.5 equiv., 3.80 mmol, 495 mg, 0.668 mL) and the mixture was stirred overnight at rt. The mixture was diluted with 100 mL EtOAc and washed with 100 mL sat. NaHCO₃(aq) and 2×100 mL 1:1 H₂O:brine. The organic extracts were dried (Na₂SO₄) and concentrated in vacuo. Purification by CombiFlash (40 g; dry load; 100:0 to 50:50 heptane:EtOAc over 20 minutes) provided 6,6-dimethyl-N-[1-(2-methylsulfanyl-1-phenyl-ethyl)pyrazol-4-yl]-1-(2-trimethylsilylethoxymethyl)-5,7-dihydro-4H-indazole-3-carboxamide (1.31 g, 2.43 mmol, 96% yield).

To a solution of 6,6-dimethyl-N-[1-(2-methylsulfanyl-1-phenyl-ethyl)pyrazol-4-yl]-1-(2-trimethylsilylethoxymethyl)-5,7-dihydro-4H-indazole-3-carboxamide (300 mg, 0.556 mmol) in tetrahydrofuran (5 mL) was added 3-chloroperbenzoic acid (2.2 equiv., 1.22 mmol, 274 mg) and the mixture was stirred for 60 minutes at rt. The mixture was diluted with 50 mL EtOAc and washed with 50 mL sat. NaHCO₃(aq) and 50 mL brine. The organic extracts were dried (Na₂SO₄) and concentrated in vacuo. Purification by CombiFlash (24 g; dry load; 70:30 to 30:70 heptane:EtOAc over 20 minutes) provided 6,6-dimethyl-N-[1-(2-methylsulfonyl-1-phenyl-ethyl)pyrazol-4-yl]-1-(2-trimethylsilylethoxymethyl)-5,7-dihydro-4H-indazole-3-carboxamide (90 mg, 0.1574 mmol, 28% yield).

To a solution of 6,6-dimethyl-N-[1-(2-methylsulfonyl-1-phenyl-ethyl)pyrazol-4-yl]-1-(2-trimethylsilylethoxymethyl)-5,7-dihydro-4H-indazole-3-carboxamide (90.0 mg, 0.157 mmol) in trifluoroacetic acid (2 mL) was added triisopropylsilane (5 equiv., 0.787 mmol, 126 mg, 0.163 mL) and the mixture was stirred for 90 minutes at rt. After in vacuo concentration, the residue was purified by reverse phase HPLC, followed by SFC on a chiral stationary phase to provide the title compounds as single enantiomers.

SFC conditions: Lux Cellulose-3 (4.6×50 mm, 5 μm particle size) at 30% methanol w/0.1% NH₄OH; 5 mL/min, 120 bars, 40° C.

64a: ¹H NMR (400 MHz, DMSO-d₆) δ 12.85-12.62 (s, 1H), 10.22-9.97 (s, 1H), 8.29-8.24 (s, 1H), 7.75-7.70 (s, 1H), 7.46-7.40 (m, 2H), 7.40-7.27 (m, 3H), 6.02-5.95 (dd, J=9.7, 3.9 Hz, 1H), 4.58-4.47 (dd, J=14.9, 9.7 Hz, 1H), 3.94-3.83 (dd, J=14.9, 4.0 Hz, 1H), 2.71-2.62 (m, 5H), 2.40-2.36 (s, 2H), 1.52-1.43 (t, J=6.4 Hz, 2H), 0.99-0.92 (s, 6H); MS: m/z=442 (M+H); SFC retention time: 0.47 min.

64b: ¹H NMR (400 MHz, DMSO-d₆) δ 12.95-12.61 (s, 1H), 10.26-9.98 (s, 1H), 8.29-8.25 (s, 1H), 7.75-7.70 (s, 1H), 7.47-7.41 (m, 2H), 7.39-7.28 (m, 3H), 6.10-5.87 (dd, J=9.7, 4.0 Hz, 1H), 4.65-4.36 (dd, J=14.9, 9.8 Hz, 1H), 4.00-3.75 (dd, J=14.7, 3.9 Hz, 1H), 2.70-2.64 (m, 4H), 2.43-2.37 (s, 2H), 1.52-1.44 (t, J=6.3 Hz, 2H), 1.00-0.93 (s, 6H); MS: m/z=442 (M+H); SFC retention time: 0.62 min.

### Examples 65a and 65b

### N-(1-benzyl-1H-pyrazol-4-yl)-1′,4,4′,5,5′,7′-hexahydro-2H-spiro[furan-3,6′-indazole]-3′-carboxamide

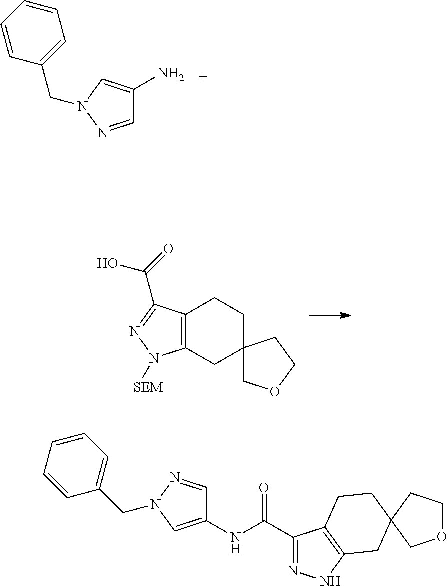 [SMILES: Nc1cnn(Cc2ccccc2)c1 ; C[Si](C)(C)CCOCn1nc(C(=O)O)c2c1CC1(CCOC1)CC2 ; O=C(Nc1cnn(Cc2ccccc2)c1)c1n[nH]c2c1CCC1(CCOC1)C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1′,4,4′,5,5′,7′-hexahydro-2H-spiro[furan-3,6′-indazole]-3′-carboxylic acid (Example C24) and 3-((4-amino-1H-pyrazol-1-yl)methyl)benzonitrile (Example A1) with 1-benzyl-1H-pyrazol-4-amine (Example A2).

SFC conditions: Lux Cellulose-3 (4.6×50 mm, 5 μm particle size) at 25% methanol w/0.1% NH₄OH; 5 mL/min, 120 bars, 40° C.

65a: ¹H NMR (400 MHz, DMSO-d₆) δ 12.88 (s, 1H), 10.09 (s, 1H), 8.07 (s, 1H), 7.64 (s, 1H), 7.39-7.18 (m, 5H), 5.27 (s, 2H), 3.80 (t, J=7.1 Hz, 2H), 3.48 (d, J=8.4 Hz, 1H), 3.42 (d, J=8.4 Hz, 1H), 2.71 (t, J=6.4 Hz, 2H), 2.61 (s, 2H), 1.82-1.60 (m, 4H); MS: m/z=378 (M+H); SFC retention time: 0.36 min.

65b: ¹H NMR (400 MHz, DMSO-d₆) δ 12.85 (s, 1H), 10.09 (s, 1H), 8.07 (s, 1H), 7.64 (s, 1H), 7.37-7.20 (m, 5H), 5.27 (s, 2H), 3.80 (t, J=7.1 Hz, 2H), 3.48 (d, J=8.4 Hz, 1H), 3.42 (d, J=8.4 Hz, 1H), 2.71 (t, J=6.4 Hz, 2H), 2.61 (s, 2H), 1.82-1.60 (m, 4H); MS: m/z=378 (M+H); SFC retention time: 0.51 min.

### Examples 66a and 66b

### 6,6-dimethyl-N-(1-((S)-morpholin-2-yl(phenyl)methyl)-1H-pyrazol-4-yl)-4,5,6,7-tetrahydro-1H-indazole-3-carboxamide

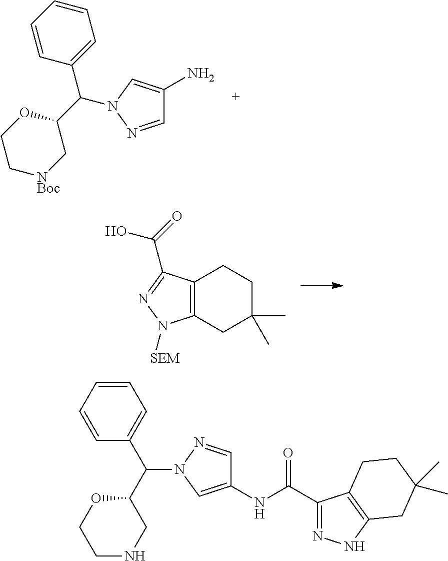

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with (2S)-tert-butyl 2-((4-amino-1H-pyrazol-1-yl)(phenyl)methyl)morpholine-4-carboxylate (Example A15).

SFC conditions: Chiralpak AD (4.6×50 mm, 5 μm particle size) at 40% methanol w/0.1% NH₄OH; 5 mL/min, 120 bars, 40° C.

66a: ¹H NMR (400 MHz, DMSO-d₆) δ 12.87-12.64 (s, 1H), 10.18-9.97 (s, 1H), 8.15-8.14 (s, 1H), 7.65-7.63 (s, 1H), 7.53-7.47 (dd, J=8.4, 1.4 Hz, 2H), 7.36-7.24 (m, 3H), 5.32-5.24 (d, J=9.3 Hz, 1H), 4.28-4.19 (td, J=8.9, 3.3 Hz, 1H), 3.72-3.62 (m, 1H), 3.45-3.35 (td, J=10.6, 3.5 Hz, 1H), 2.70-2.57 (m, 3H), 2.44-2.29 (m, 3H), 1.50-1.42 (t, J=6.4 Hz, 2H), 1.00-0.92 (s, 6H); MS: m/z=435 (M+H); SFC retention time: 0.56 min.

66b: ¹H NMR (400 MHz, DMSO-d₆) δ 12.91-12.60 (s, 1H), 10.08-9.91 (s, 1H), 8.14-8.12 (s, 1H), 7.64-7.60 (s, 1H), 7.47-7.41 (m, 2H), 7.36-7.25 (m, 3H), 5.35-5.27 (d, J=8.8 Hz, 1H), 4.30-4.20 (td, J=9.0, 3.0 Hz, 1H), 3.76-3.67 (dt, J=10.9, 2.2 Hz, 1H), 3.45-3.35 (td, J=10.8, 3.3 Hz, 1H), 2.71-2.54 (m, 3H), 2.41-2.26 (m, 3H), 1.53-1.41 (t, J=6.4 Hz, 2H), 1.01-0.91 (s, 6H); MS: m/z=435 (M+H); SFC retention time: 0.87 min.

### Examples 67a and 67b

### 6,6-dimethyl-N-(1-((1-(methylsulfonyl)piperidin-4-yl)(phenyl)methyl)-1H-pyrazol-4-yl)-4,5,6,7-tetrahydro-1H-indazole-3-carboxamide

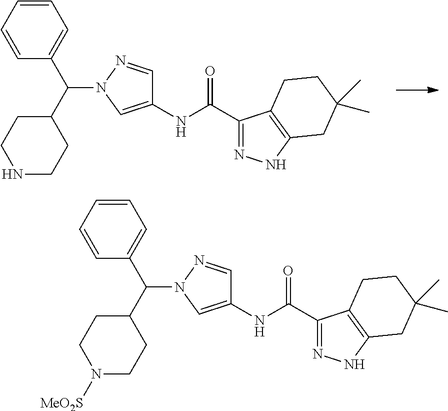 [SMILES: CC1(C)CCc2c(C(=O)Nc3cnn(C(c4ccccc4)C4CCNCC4)c3)n[nH]c2C1 ; CC1(C)CCc2c(C(=O)Nc3cnn(C(c4ccccc4)C4CCN(S(C)(=O)=O)CC4)c3)n[nH]c2C1]

Prepared in an analogous manner to 6,6-dimethyl-N-(1-(3-(N-methylmethylsulfonamido)-1-phenylpropyl)-1H-pyrazol-4-yl)-4,5,6,7-tetrahydro-1H-indazole-3-carboxamide (Example 52), replacing 6,6-dimethyl-N-(1-(3-(methylamino)-1-phenylpropyl)-1H-pyrazol-4-yl)-4,5,6,7-tetrahydro-1H-indazole-3-carboxamide (Example 39a) with 6,6-dimethyl-N-(1-(phenyl(piperidin-4-yl)methyl)-1H-pyrazol-4-yl)-4,5,6,7-tetrahydro-1H-indazole-3-carboxamide (Examples 40a and 40b). Also, the stereoisomers were separated by preparative chiral HPLC instead of SFC.

Chiral HPLC Conditions: ChiralPak IC-3 (4.6×50 mm, 3 μm particle size); eluent=Hex (0.1% Et₃N):IPA 50:50; 1.0 ml/min, 4.8 MPA, 25° C.

67a: ¹H-NMR (CD₃OD, 300 MHz, ppm) δ 8.15 (s, 1H), 7.69 (s, 1H), 7.52-7.55 (m, 2H), 7.29-7.40 (m, 3H), 5.03 (s, 1H), 3.66-3.74 (m, 2H), 2.58-2.83 (m, 7H), 2.44 (s, 2H), 1.30-1.60 (m, 7H), 1.04 (s, 6H); MS: m/z=511 (M+H); HPLC retention time: 6.57 min.

67b: ¹H-NMR (CD₃OD, 300 MHz, ppm) δ 8.11 (s, 1H), 7.65 (s, 1H), 7.47-7.50 (m, 2H), 7.24-7.35 (m, 3H), 4.95-4.98 (m, 1H), 3.50-3.70 (m, 2H), 3.27-3.32 (m, 1H), 2.53-2.78 (m, 7H), 2.39 (s, 2H), 1.12-1.55 (m, 7H), 0.99 (s, 6H); MS: m/z=511 (M+H); HPLC retention time: 14.45 min.

### Examples 68a and 68b

### N-(1-(2-(dimethylamino)-1-phenylethyl)-1H-pyrazol-4-yl)-5a-methyl-1,4,4a,5,5a,6-hexahydrocyclopropa[f]indazole-3-carboxamide

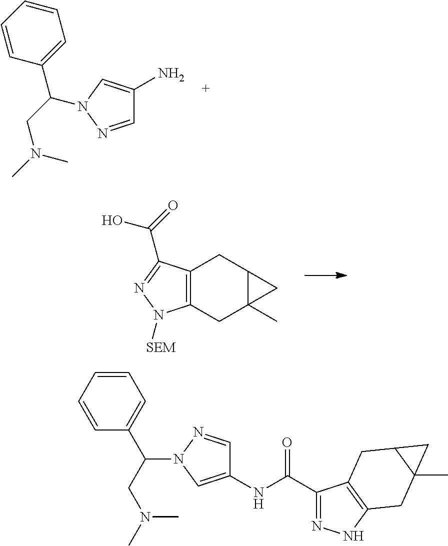

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5a-methyl-1-((2-(trimethylsilyl)ethoxy)methyl)-1,4,4a,5,5a,6-hexahydrocyclopropa[f]indazole-3-carboxylic acid (Example C31a) and 3-((4-amino-1H-pyrazol-1-yl)methyl)benzonitrile (Example A1) with 1-(2-(dimethylamino)-1-phenylethyl)-1H-pyrazol-4-amine (Example A5). Also, the stereoisomers were separated by preparative chiral HPLC instead of SFC. Chiral HPLC Conditions: ChiralPak IA-3 (4.6×50 mm, 3 μm particle size); eluent=Hex (0.1% Et₃N):IPA 80:20; 1.0 ml/min, 3.4 MPA, 25° C.

68a: ¹H-NMR (300 MHz, CD₃OD) δ 8.00 (s, 1H), 7.51 (s, 1H), 7.18-7.07 (m, 5H), 5.42-5.37 (q, J=5.0 Hz, 1H), 3.36-3.06 (m, 2H), 2.89-2.49 (m, 4H), 2.12 (s, 6H), 1.07 (s, 3H), 0.92-0.89 (m, 1H), 0.22-0.18 (m, 1H), 0.02-0.02 (m, 1H). MS: m/z=405 (M+H); HPLC retention time: 2.88 min.

68b: ¹H-NMR (300 MHz, CD₃OD) δ 8.00 (s, 1H), 7.52 (s, 1H), 7.16-7.10 (m, 5H), 5.44-5.40 (q, J=4.6 Hz, 1H), 3.40-3.03 (m, 2H), 2.89-2.50 (m, 4H), 2.13 (s, 6H), 1.08 (s, 3H), 0.93-0.87 (m, 1H), 0.22-0.18 (m, 1H), 0.01-0.02 (m, 1H). MS: m/z=405 (M+H); HPLC retention time: 5.94 min.

### Examples 69a and 69b

### 5a-methyl-N-(1-(3-(methylsulfonyl)-1-phenylpropyl)-1H-pyrazol-4-yl)-1,4,4a,5,5a,6-hexahydrocyclopropa[f]indazole-3-carboxamide

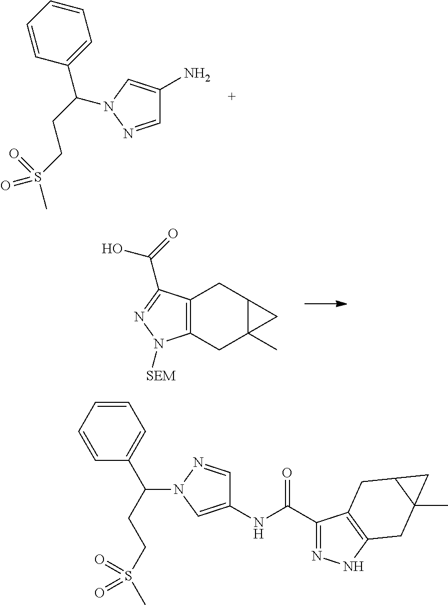

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5a-methyl-1-((2-(trimethylsilyl)ethoxy)methyl)-1,4,4a,5,5a,6-hexahydrocyclopropa[f]indazole-3-carboxylic acid (Example C31a) and 3-((4-amino-1H-pyrazol-1-yl)methyl)benzonitrile (Example A1) with 1-(3-(methylsulfonyl)-1-phenylpropyl)-1H-pyrazol-4-amine (Example A53). Also, the stereoisomers were separated by preparative chiral HPLC instead of SFC.

Chiral HPLC Conditions: ChiralPak IB (4.6×250 mm, 5 μm particle size); eluent=Hex (0.1% Et₃N):EtOH 80:20; 1.0 ml/min, 6.1 MPA, 25° C.

69a: ¹H-NMR (300 MHz, DMSO-d₆) δ 10.13 (s, 1H), 8.10 (s, 1H), 7.71 (s, 1H), 7.39-7.28 (m, 5H), 5.56-5.51 (q, J=5.2 Hz, 1H), 3.22-3.16 (m, 2H), 3.00 (s, 3H), 2.93-2.64 (m, 6H), 1.20 (s, 3H), 1.03-0.99 (m, 1H), 0.36-0.32 (m, 1H), 0.11-0.05 (m, 1H). MS: m/z=455 (M+H); HPLC retention time: 18.02 min.

69b: ¹H-NMR (300 MHz, CD₃OD): δ 7.90 (s, 1H), 7.55 (s, 1H), 7.19-7.11 (m, 5H), 5.41-5.36 (q, J=5.2 Hz, 1H), 2.99-2.40 (m, 11H), 1.08 (s, 3H), 0.94-0.88 (m, 1H), 0.23-0.18 (m, 1H), 0.18-0.02 (m, 1H). MS: m/z=455 (M+H); HPLC retention time: 15.04 min.

### Examples 70a and 70b

### N-(1-benzyl-1H-pyrazol-4-yl)-5-hydroxy-6,6-dimethyl-4,5,6,7-tetrahydro-1H-indazole-3-carboxamide

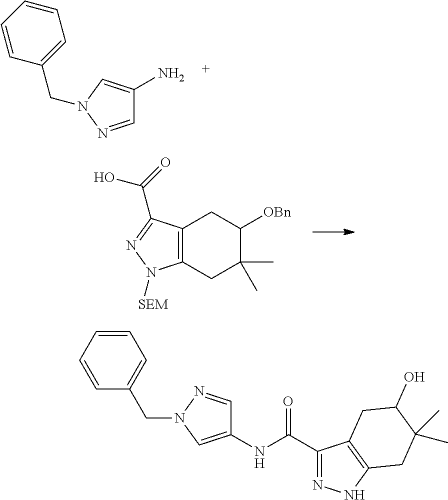

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5-(benzyloxy)-6,6-dimethyl-1-((2-(trimethylsilyl)ethoxy)methyl)-4,5,6,7-tetrahydro-1H-indazole-3-carboxylic acid (Example C34) and 3-((4-amino-1H-pyrazol-1-yl)methyl)benzonitrile (Example A1) with 1-benzyl-1H-pyrazol-4-amine (Example A2), providing the intermediate benzyl protected N-(1-benzyl-1H-pyrazol-4-yl)-5-(benzyloxy)-6,6-dimethyl-4,5,6,7-tetrahydro-1H-indazole-3-carboxamide. Debenzylation was accomplished using palladium on carbon in EtOH under an atmosphere of hydrogen (1 atm). Also, the stereoisomers were separated by preparative chiral HPLC instead of SFC.

Chiral HPLC Conditions: ChiralPak IB (4.6×250 mm, 5 μm particle size); eluent=Hex (0.1% Et₃N):EtOH 80:20; 1.0 ml/min, 4.0 MPA, 25° C.

70a: ¹H NMR (300 MHz, CD₃OD) δ 8.04 (s, 1H), 7.69 (s, 1H), 7.38-7.25 (m, 5H), 5.32 (s, 2H), 3.65 (t, J=5.2 Hz, 1H), 3.04 (dd, J=4.5 Hz, J=16.8 Hz, 1H), 2.79 (dd, J=5.7 Hz, J=16.8 Hz, 1H), 2.62 (d, J=15.9 Hz, 1H), 2.41 (d, J=16.2 Hz), 1.04 (s, 3H), 1.00 (s, 3H); MS: m/z=366 (M+H); HPLC retention time: 8.21 min.

70b: ¹H NMR (300 MHz, CD₃OD): δ 8.04 (s, 1H), 7.69 (s, 1H), 7.38-7.24 (m, 5H), 5.32 (s, 2H), 3.65 (t, J=5.1 Hz, 1H), 3.04 (dd, J=4.5 Hz, J=17.0 Hz, 1H), 2.79 (dd, J=5.7 Hz, J=16.8 Hz, 1H), 2.62 (d, J=16.2 Hz, 1H), 2.41 (d, J=16.2 Hz), 1.04 (s, 3H), 1.00 (s, 3H); MS: m/z=366 (M+H); HPLC retention time: 10.89 min.

### Examples 71a and 71b

### N-(1-benzyl-1H-pyrazol-4-yl)-7,7-dimethyl-4,5,7,8-tetrahydro-1H-oxepino[4,5-c]pyrazole-3-carboxamide and

### N-(1-benzyl-1H-pyrazol-4-yl)-5,5-dimethyl-4,5,7,8-tetrahydro-1H-oxepino[4,5-c]pyrazole-3-carboxamide

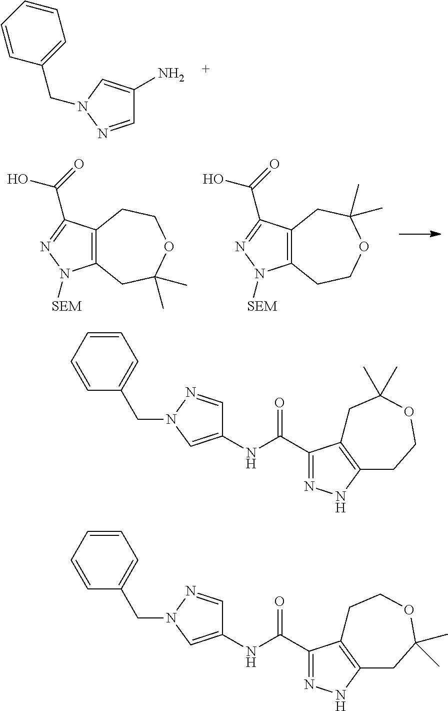

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 7,7-dimethyl-1-((2-(trimethylsilyl)ethoxy)methyl)-4,5,7,8-tetrahydro-1H-oxepino[4,5-c]pyrazole-3-carboxylic acid and 5,5-dimethyl-1-((2-(trimethylsilyl)ethoxy)methyl)-4,5,7,8-tetrahydro-1H-oxepino[4,5-c]pyrazole-3-carboxylic acid (Example C35a and b) and 3-((4-amino-1H-pyrazol-1-yl)methyl)benzonitrile (Example A1) with 1-benzyl-1H-pyrazol-4-amine (Example A2).

71a: ¹H NMR (300 MHz, CDCl₃) δ 8.66 (s, 1H), 8.03 (s, 1H), 7.54 (s, 1H), 7.33-7.26 (m, 5H), 5.27 (s, 2H), 3.91 (t, J=4.8, 2H), 3.29 (s, 2H), 2.90 (t, J=4.8, 2H), 1.24 (s, 6H). MS: m/z=366 (M+H).

71b: ¹H NMR (300 MHz, CDCl₃) δ 8.65 (s, 1H), 8.02 (s, 1H), 7.54 (s, 1H), 7.32-7.22 (m, 5H), 5.27 (s, 2H), 3.88 (t, J=4.8, 2H), 3.19 (s, 2H), 2.90 (t, J=4.8, 2H), 1.23 (s, 6H). MS: m/z=366 (M+H).

### Example 72

### N-(1-(2-(dimethylamino)ethyl)-1H-pyrazol-4-yl)-6,6-dimethyl-4,5,6,7-tetrahydro-1H-indazole-3-carboxamide

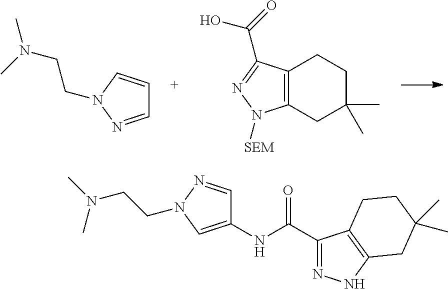

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(2-(dimethylamino)ethyl)-1H-pyrazol-4-amine (Example A31).

72a: ¹H NMR (400 MHz, DMSO-d6) δ 12.78 (s, 1H), 10.01 (s, 1H), 8.01 (s, 1H), 7.59 (s, 1H), 4.13 (t, J=6.5 Hz, 2H), 2.67 (s, 2H), 2.60 (t, J=6.5 Hz, 2H), 2.39 (s, 2H), 2.16 (s, 6H), 1.48 (t, J=6.4 Hz, 2H), 0.97 (s, 6H); MS: m/z=331 (M+H).

### Examples 73a and 73b

### 6,6-dimethyl-N-(1-(2-(methylsulfonyl)-1-(pyridin-3-yl)ethyl)-1H-pyrazol-4-yl)-4,5,6,7-tetrahydro-1H-indazole-3-carboxamide

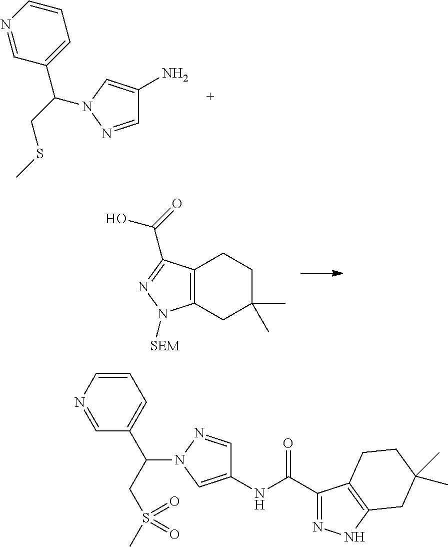

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with 1-(2-(methylthio)-1-(pyridin-3-yl)ethyl)-1H-pyrazol-4-amine (Example A26). It is important to note that the mCPBA oxidation needs to be monitored closely by LCMS to prevent over-oxidation to the pyridine N-oxide (generally <15 min. reaction time).

SFC conditions: Lux Cellulose-1 (4.6×50 mm, 5 μm particle size) at 30% methanol w/0.1% NH₄OH; 4 mL/min, 120 bars, 40° C.

73a: ¹H NMR (400 MHz, DMSO-d₆) δ 12.80 (s, 1H), 10.15 (s, 1H), 8.67 (d, J=2.1 Hz, 1H), 8.51 (dd, J=4.8, 1.6 Hz, 1H), 8.33 (s, 1H), 7.88-7.84 (m, 1H), 7.74 (s, 1H), 7.38 (dd, J=8.0, 4.8 Hz, 1H), 6.10 (dd, J=9.6, 4.3 Hz, 1H), 4.53 (dd, J=14.8, 9.6 Hz, 1H), 4.00 (dd, J=15.0, 4.3 Hz, 1H), 2.71 (s, 3H), 2.67 (t, J=6.2 Hz, 2H), 2.39 (s, 2H), 1.47 (t, J=6.3 Hz, 2H), 0.96 (s, 6H); MS: m/z=443 (M+H); SFC retention time: 0.79 min.

73b: ¹H NMR (400 MHz, DMSO-d₆) δ 12.80 (s, 1H), 10.14 (s, 1H), 8.67 (d, J=2.2 Hz, 1H), 8.51 (dd, J=4.8, 1.6 Hz, 1H), 8.33 (s, 1H), 7.89-7.84 (m, 1H), 7.74 (s, 1H), 7.38 (dd, J=8.0, 4.8 Hz, 1H), 6.10 (dd, J=9.5, 4.3 Hz, 1H), 4.53 (dd, J=14.8, 9.6 Hz, 1H), 4.00 (dd, J=14.8, 4.2 Hz, 1H), 2.71 (s, 3H), 2.67 (t, J=6.0 Hz, 2H), 2.39 (s, 2H), 1.47 (t, J=6.4 Hz, 2H), 0.96 (s, 6H). MS: m/z=443 (M+H); SFC retention time: 1.15 min.

### Examples 74a and 74b

### N-(1-((1,1-dioxidotetrahydro-2H-thiopyran-4-yl)(pyridin-3-yl)methyl)-1H-pyrazol-4-yl)-6,6-dimethyl-4,5,6,7-tetrahydro-1H-indazole-3-carboxamide

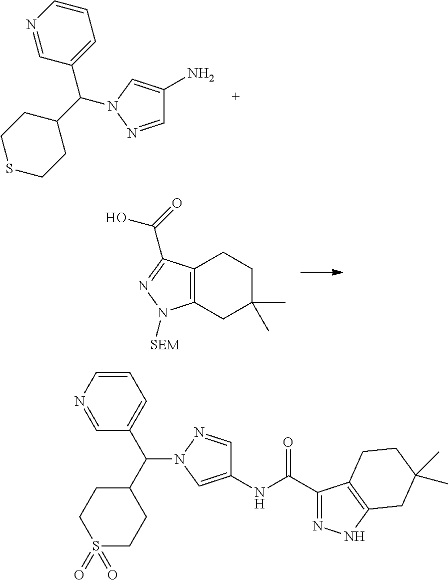

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with 1-(pyridin-3-yl(tetrahydro-2H-thiopyran-4-yl)methyl)-1H-pyrazol-4-amine (Example A16). It is important to note that the mCPBA oxidation needs to be monitored closely by LCMS to prevent over-oxidation to the pyridine N-oxide (generally <15 min. reaction time).

SFC conditions: Lux Cellulose-1 (4.6×50 mm, 5 μm particle size) at 45% methanol w/0.1% NH₄OH; 4 mL/min, 120 bars, 40° C.

74a: ¹H NMR (400 MHz, DMSO-d₆) δ 12.93-12.66 (s, 1H), 10.21-10.01 (s, 1H), 8.76-8.72 (dd, J=2.3, 0.9 Hz, 1H), 8.54-8.49 (dd, J=4.8, 1.6 Hz, 1H), 8.21-8.19 (d, J=0.7 Hz, 1H), 8.01-7.94 (dt, J=7.9, 1.9 Hz, 1H), 7.73-7.70 (s, 1H), 7.43-7.38 (ddd, J=7.9, 4.9, 0.8 Hz, 1H), 5.50-5.33 (d, J=10.8 Hz, 1H), 3.19-2.75 (m, 5H), 2.71-2.61 (m, 2H), 2.42-2.35 (s, 2H), 1.76-1.50 (m, 4H), 1.50-1.43 (t, J=6.4 Hz, 2H), 1.00-0.92 (s, 6H); MS: m/z=483 (M+H); SFC retention time: 0.62 min.

74b: ¹H NMR (400 MHz, DMSO-d₆) δ 12.93-12.66 (s, 1H), 10.21-10.01 (s, 1H), 8.76-8.72 (dd, J=2.3, 0.9 Hz, 1H), 8.54-8.49 (dd, J=4.8, 1.6 Hz, 1H), 8.21-8.19 (d, J=0.7 Hz, 1H), 8.01-7.94 (dt, J=7.9, 1.9 Hz, 1H), 7.73-7.70 (s, 1H), 7.43-7.38 (ddd, J=7.9, 4.9, 0.8 Hz, 1H), 5.50-5.33 (d, J=10.8 Hz, 1H), 3.19-2.75 (m, 5H), 2.71-2.61 (m, 2H), 2.42-2.35 (s, 2H), 1.76-1.50 (m, 4H), 1.50-1.43 (t, J=6.4 Hz, 2H), 1.00-0.92 (s, 6H); MS: m/z=483 (M+H); SFC retention time: 1.44 min.

### Examples 75a and 75b

### N-(1-((1-(2-fluoroethyl)azetidin-3-yl)(phenyl)methyl)-1H-pyrazol-4-yl)-6,6-dimethyl-4,5,6,7-tetrahydro-1H-indazole-3-carboxamide

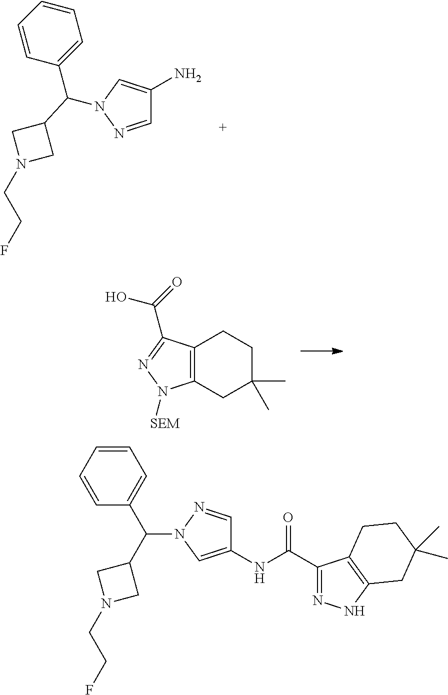

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-((1-(2-fluoroethyl)azetidin-3-yl)(phenyl)methyl)-1H-pyrazol-4-amine (Example A56). Also, the stereoisomers were separated by preparative chiral HPLC instead of SFC.

Chiral HPLC Conditions: ChiralPak IC-3 (4.6×150 mm, 3 μm particle size); eluent=Hex (0.1% Et₃N):EtOH 70:30; 1.0 ml/min, 7.8 MPA, 25° C.

75a: ¹H NMR (300 MHz, CD₃OD) δ 8.06 (s, 1H), 7.68 (s, 1H), 7.45-7.26 (m, 5H), 5.60 (d, J=10.2, 1H), 4.52 (s, 1H), 4.37 (s, 1H), 3.65-3.53 (m, 2H), 3.44 (t, J=7.05, 1H), 3.17-3.09 (m, 2H), 2.87-2.78 (m, 4H), 2.44 (s, 2H), 1.57 (t, J=5.8, 2H), 1.04 (s, 6H); MS: m/z=451 (M+H); HPLC retention time: 4.85 min.

75b: ¹H NMR (300 MHz, CD₃OD) δ 8.06 (s, 1H),7.68 (s, 1H), 7.43-7.36 (m, 5H), 5.59 (d, J=10.2, 1H), 4.53 (s, 1H), 4.37 (s, 1H), 3.63-3.51 (m, 2H), 3.42 (d, J=6.3, 1H), 3.18-3.10 (m, 2H), 2.91-2.79 (m, 4H), 2.44 (s, 2H), 1.59 (t, J=5.8, 2H), 1.04 (s, 6H); MS: m/z=451 (M+H); HPLC retention time: 3.88 min.

### Example 76

### 6,6-dimethyl-N-(1-(2-(2-oxopyrrolidin-1-yl)-1-phenylethyl)-1H-pyrazol-4-yl)-4,5,6,7-tetrahydro-1H-indazole-3-carboxamide

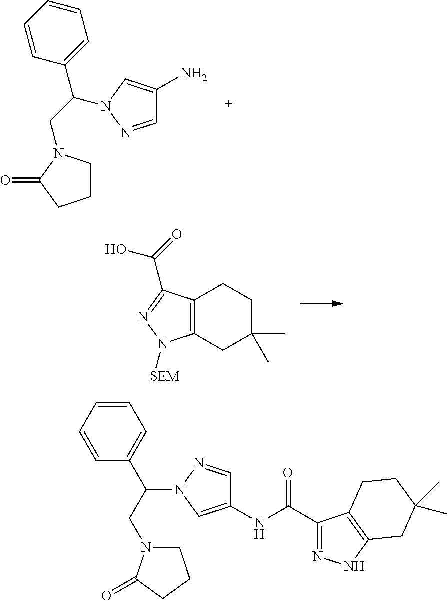

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(2-(4-amino-1H-pyrazol-1-yl)-2-phenylethyl)pyrrolidin-2-one (Example A58). SFC resolution of the enantiomers proved unsuccessful, so Example 76 was tested as the racemic mixture.

76: ¹H-NMR (300 MHz, DMSO-d₆): δ 12.82 (s, 1H), 10.13 (s, 1H), 8.14 (s, 1H), 7.71 (s, 1H), 7.28-7.41 (m, 5H), 5.59-5.64 (m, 1H), 3.89-4.03 (m, 2H), 3.10-3.18 (m, 1H), 2.84-2.92 (m, 1H), 2.64-2.68 (m, 2H), 2.39-2.50 (m, 2H), 2.07-2.15 (m, 2H), 1.75-7.81 (m, 2H), 1.401-1.555 (m, 2H), 0.95-1.01 (s, 6H); MS: m/z=447 (M+H).

### Example 77a and 77b

### 6,6-dimethyl-N-(1-(2-(3-oxomorpholino)-1-phenylethyl)-1H-pyrazol-4-yl)-4,5,6,7-tetrahydro-1H-indazole-3-carboxamide

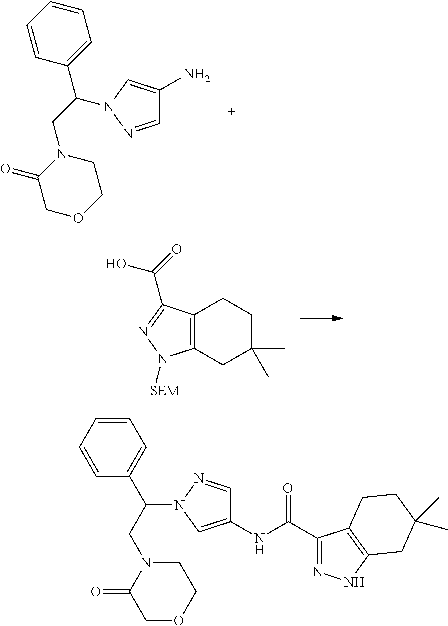

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 4-(2-(4-amino-1H-pyrazol-1-yl)-2-phenylethyl)morpholin-3-one (Example A59). Also, the stereoisomers were separated by preparative chiral HPLC instead of SFC.

Chiral HPLC Conditions: ChiralPak IA (4.6×250 mm, 3 μm particle size); eluent=Hex (0.1% Et₃N):EtOH 80:20; 1.0 ml/min, 2.0 MPA, 25° C.

77a: ¹H NMR (300 MHz, CD₃OD) δ 8.12 (s, 1H), 7.77 (s, 1H), 7.47-7.34 (m, 5H), 5.77 (q, J=5.0 Hz, 1H), 4.30 (dd, J=5.0 Hz, J=13.6 Hz, 1H), 4.12-4.05 (m, 3H), 3.72-3.65 (m, 2H), 3.31-3.23 (m, 1H), 2.94-2.89 (m, 1H), 2.79 (t, J=6.4 Hz, 2H), 2.44 (s, 2H), 1.60-1.56 (t, J=6.3 Hz, 2H), 1.04 (s, 3H); MS: m/z=463 (M+H); HPLC retention time: 16.68 min.

77b: ¹H NMR (300 MHz, CD₃OD) δ 8.12 (s, 1H), 7.77 (s, 1H), 7.47-7.34 (m, 5H), 5.77 (q, J=5.1 Hz, 1H), 4.30 (dd, J=4.8 Hz, J=13.8 Hz, 1H), 4.12-4.05 (m, 3H), 3.72-3.65 (m, 2H), 3.28-3.23 (m, 1H), 2.95-2.89 (m, 1H), 2.79 (t, J=6.0 Hz, 2H), 2.44 (s, 2H), 1.60-1.56 (t, J=6.3 Hz, 2H), 1.04 (s, 3H); MS: m/z=463 (M+H); HPLC retention time: 19.49 min.

### Example 78a and 78b

### 6,6-dimethyl-N-(1-(1-(tetrahydro-2H-pyran-4-yl)ethyl)-1H-pyrazol-4-yl)-4,5,6,7-tetrahydro-1H-indazole-3-carboxamide

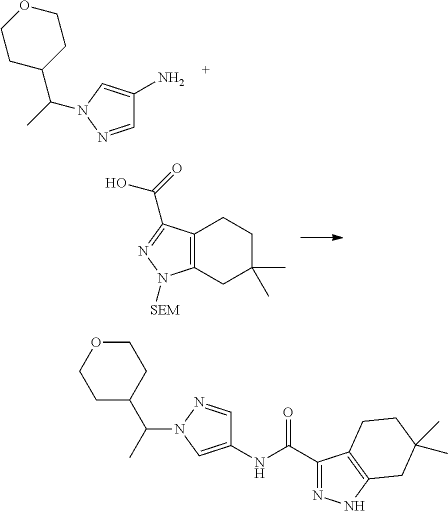

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(tetrahydro-2H-pyran-4-yl)ethyl)-1H-pyrazol-4-amine (Example A30).

SFC conditions: Lux Cellulose-3 (4.6×50 mm, 5 μm particle size) at 15% methanol w/0.1% NH₄OH; 4 mL/min, 120 bars, 40° C.

78a: ¹H NMR (400 MHz, DMSO-d6) δ 12.77 (s, 1H), 10.01 (s, 1H), 7.99 (s, 1H), 7.60 (s, 1H), 4.05 (dt, J=8.5, 6.8 Hz, 1H), 3.93-3.68 (m, 2H), 3.15 (td, J=11.8, 2.2 Hz, 1H), 2.67 (t, J=6.4 Hz, 2H), 2.39 (s, 2H), 1.97-1.77 (m, 1H), 1.60 (d, J=13.5 Hz, 1H), 1.47 (t, J=6.4 Hz, 2H), 1.38 (d, J=6.8 Hz, 3H), 1.31-1.02 (m, 3H), 0.97 (s, 6H); MS: m/z=372 (M+H); SFC retention time: 0.58 min.

78b: ¹H NMR (400 MHz, DMSO-d6) δ 12.78 (s, 1H), 10.01 (s, 1H), 7.99 (d, J=0.7 Hz, 1H), 7.61 (s, 1H), 4.10-3.98 (m, 1H), 3.92-3.81 (m, 1H), 3.81-3.72 (m, 1H), 3.15 (td, J=11.7, 2.2 Hz, 1H), 2.67 (t, J=6.1 Hz, 2H), 2.39 (s, 3H), 1.96-1.81 (m, 1H), 1.60 (d, J=13.3 Hz, 1H), 1.47 (t, J=6.4 Hz, 2H), 1.38 (d, J=6.8 Hz, 4H), 1.31-1.03 (m, 3H), 0.97 (s, 8H); MS: m/z=372 (M+H); SFC retention time: 0.42 min.

### Examples 79a and 79b

### N-(1-benzyl-1H-pyrazol-4-yl)-4,4′,5′,6-tetrahydro-1H,2′H-spiro[cyclopenta[c]pyrazole-5,3′-furan]-3-carboxamide

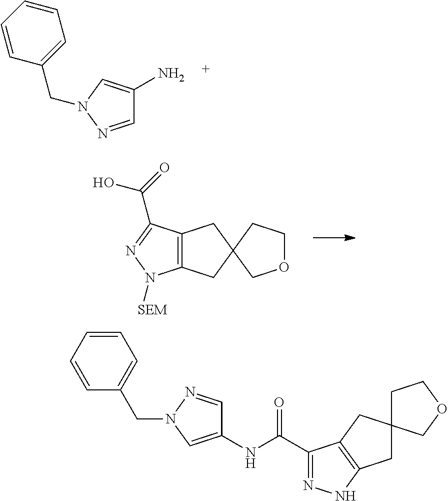

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-4,4′,5′,6-tetrahydro-1H,2′H-spiro[cyclopenta[c]pyrazole-5,3′-furan]-3-carboxylic acid (Example C26) and 3-((4-amino-1H-pyrazol-1-yl)methyl)benzonitrile (Example A1) with 1-benzyl-1H-pyrazol-4-amine (Example A2).

SFC conditions: Lux Cellulose-3 (4.6×50 mm, 5 μm particle size) at 20% methanol w/0.1% NH₄OH; 4 mL/min, 120 bars, 40° C.

79a: ¹H NMR (400 MHz, DMSO-d₆) δ 12.91 (s, 1H), 10.11 (s, 1H), 8.05 (s, 1H), 7.64 (s, 1H), 7.37-7.26 (m, 3H), 7.26-7.20 (m, 2H), 5.28 (s, 2H), 3.83 (t, J=7.0 Hz, 2H), 3.68-3.56 (m, 2H), 2.91-2.68 (m, 4H), 2.05-1.95 (m, 2H); MS: m/z=364 (M+H); SFC retention time: 0.95 min.

79b: ¹H NMR (400 MHz, DMSO-d₆) δ 12.91 (s, 1H), 10.11 (s, 1H), 8.05 (s, 1H), 7.64 (s, 1H), 7.37-7.26 (m, 3H), 7.25-7.20 (m, 2H), 5.28 (s, 2H), 3.83 (t, J=7.0 Hz, 2H), 3.68-3.56 (m, 2H), 2.93-2.69 (m, 4H), 2.04-1.94 (m, 2H); MS: m/z=364 (M+H); SFC retention time: 1.07 min.

### Examples 80a and 80b

### N-(1-(3-cyano-1-(pyridin-3-yl)propyl)-1H-pyrazol-4-yl)-6,6-dimethyl-4,5,6,7-tetrahydro-1H-indazole-3-carboxamide

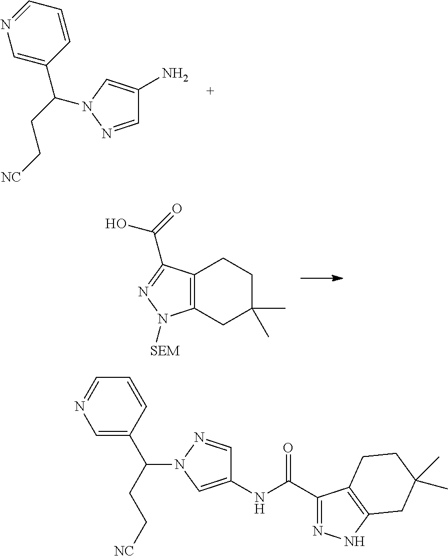

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 4-(4-amino-1H-pyrazol-1-yl)-4-(pyridin-3-yl)butanenitrile (Example A32).

SFC conditions: Lux Cellulose-1 (4.6×50 mm, 5 μm particle size) at 35% methanol w/0.1% NH₄OH; 4 mL/min, 120 bars, 40° C.

80a: ¹H NMR (400 MHz, DMSO-d₆) δ 12.79 (s, 1H), 10.12 (s, 1H), 8.61-8.54 (m, 1H), 8.50 (dd, J=4.8, 1.6 Hz, 1H), 8.22 (d, J=0.6 Hz, 1H), 7.80-7.71 (m, 2H), 7.38 (ddd, J=8.0, 4.8, 0.9 Hz, 1H), 5.55 (dd, J=10.0, 4.5 Hz, 2H), 2.81-2.69 (m, 1H), 2.69-2.60 (m, 2H), 2.45-2.34 (m, 5H), 1.47 (t, J=6.4 Hz, 2H), −0.00 (s, 4H); MS: m/z=404 (M+H); SFC retention time: 0.89 min.

80b: ¹H NMR (400 MHz, DMSO-d₆) δ 12.79 (s, 1H), 10.12 (s, 1H), 8.59-8.55 (m, 1H), 8.50 (dd, J=4.8, 1.6 Hz, 1H), 8.22 (d, J=0.7 Hz, 1H), 7.81-7.70 (m, 2H), 7.38 (ddd, J=7.9, 4.8, 0.8 Hz, 1H), 5.55 (dd, J=10.1, 4.5 Hz, 1H), 2.72 (s, 1H), 2.69-2.62 (m, 2H), 2.44-2.36 (m, 4H), 1.47 (t, J=6.4 Hz, 2H), 0.96 (s, 6H); MS: m/z=404 (M+H); SFC retention time: 0.47 min.

### Examples 81a and 81b

### N-(1-(2-hydroxy-1-(pyridin-3-yl)ethyl)-1H-pyrazol-4-yl)-6,6-dimethyl-4,5,6,7-tetrahydro-1H-indazole-3-carboxamide

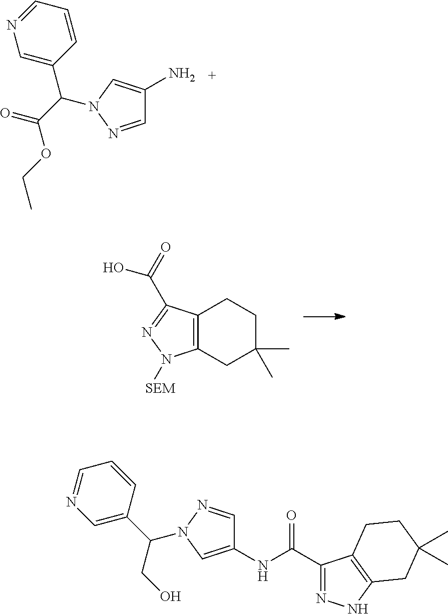 [SMILES: CCOC(=O)C(c1cccnc1)n1cc(N)cn1 ; CC1(C)CCc2c(C(=O)O)nn(COCC[Si](C)(C)C)c2C1 ; CC1(C)CCc2c(C(=O)Nc3cnn(C(CO)c4cccnc4)c3)n[nH]c2C1]

Prepared in an analogous manner to N-(1-(2-hydroxy-1-phenylethyl)-1H-pyrazol-4-yl)-6,6-dimethyl-4,5,6,7-tetrahydro-1H-indazole-3-carboxamide (Examples 53a and 53b), replacing ethyl 2-(4-aminopyrazol-1-yl)-2-phenyl-acetate (Example A11) with ethyl 2-(4-amino-1H-pyrazol-1-yl)-2-(pyridin-3-yl)acetate (Example A17).

SFC conditions: Chiralpak AS (4.6×50 mm, 5 μm particle size) at 25% methanol w/0.1% NH₄OH; 4 mL/min, 120 bars, 40° C.

81a: ¹H NMR (400 MHz, DMSO-d₆) δ 12.87-12.72 (s, 1H), 10.14-9.99 (s, 1H), 8.54-8.51 (d, J=2.2 Hz, 1H), 8.50-8.46 (dd, J=4.8, 1.7 Hz, 1H), 8.20-8.18 (s, 1H), 7.72-7.66 (m, 2H), 7.38-7.33 (ddd, J=7.8, 4.8, 0.8 Hz, 1H), 5.56-5.35 (dd, J=7.8, 5.6 Hz, 1H), 5.25-5.06 (t, J=5.4 Hz, 1H), 4.24-4.13 (ddd, J=11.2, 7.9, 5.6 Hz, 1H), 4.05-3.93 (m, 1H), 2.71-2.62 (t, J=7.0, 5.4 Hz, 2H), 2.41-2.36 (s, 2H), 1.52-1.43 (t, J=6.3 Hz, 2H), 1.00-0.93 (s, 6H); MS: m/z=381 (M+H); SFC retention time: 0.36 min.

81b: ¹H NMR (400 MHz, DMSO-d₆) δ 12.87-12.72 (s, 1H), 10.14-9.99 (s, 1H), 8.54-8.51 (d, J=2.2 Hz, 1H), 8.50-8.46 (dd, J=4.8, 1.7 Hz, 1H), 8.20-8.18 (s, 1H), 7.72-7.66 (m, 2H), 7.38-7.33 (ddd, J=7.8, 4.8, 0.8 Hz, 1H), 5.56-5.35 (dd, J=7.8, 5.6 Hz, 1H), 5.25-5.06 (t, J=5.4 Hz, 1H), 4.24-4.13 (ddd, J=11.2, 7.9, 5.6 Hz, 1H), 4.05-3.93 (m, 1H), 2.71-2.62 (t, J=7.0, 5.4 Hz, 2H), 2.41-2.36 (s, 2H), 1.52-1.43 (t, J=6.3 Hz, 2H), 1.00-0.93 (s, 6H); MS: m/z=381 (M+H); SFC retention time: 0.49 min.

### Examples 82

### N-(1-benzyl-1H-pyrazol-4-yl)-4,6-dihydro-1H-spiro[cyclopenta[c]pyrazole-5,3′-oxetane]-3-carboxamide

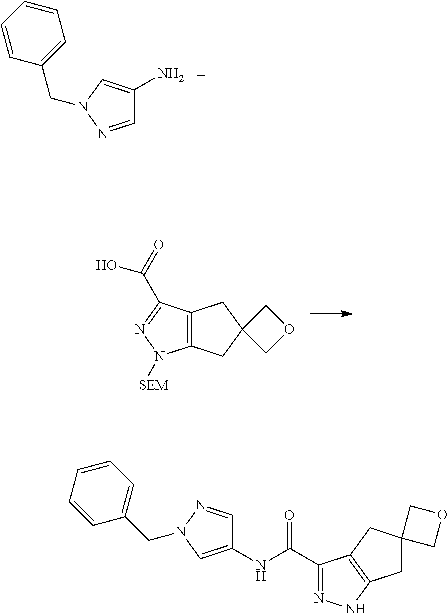 [SMILES: Nc1cnn(Cc2ccccc2)c1 ; C[Si](C)(C)CCOCn1nc(C(=O)O)c2c1CC1(COC1)C2 ; O=C(Nc1cnn(Cc2ccccc2)c1)c1n[nH]c2c1CC1(COC1)C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-4,6-dihydro-1H-spiro[cyclopenta[c]pyrazole-5,3′-oxetane]-3-carboxylic acid (Example C28) and 3-((4-amino-1H-pyrazol-1-yl)methyl)benzonitrile (Example A1) with 1-benzyl-1H-pyrazol-4-amine (Example A2).

82: ¹H NMR (400 MHz, DMSO-d₆) δ 12.94 (s, 1H), 10.12 (s, 1H), 8.06 (s, 1H), 7.64 (s, 1H), 7.38-7.26 (m, 3H), 7.26-7.20 (m, 2H), 5.28 (s, 2H), 4.64-4.55 (m, 4H), 3.24-2.97 (m, 4H). MS: m/z=350 (M+H).

### Examples 83a and 83b

### N-(1-((1,1-dioxidotetrahydro-2H-thiopyran-4-yl)(phenyl)methyl)-1H-pyrazol-4-yl)-5a-methyl-1,4,4a,5,5a,6-hexahydrocyclopropa[f]indazole-3-carboxamide

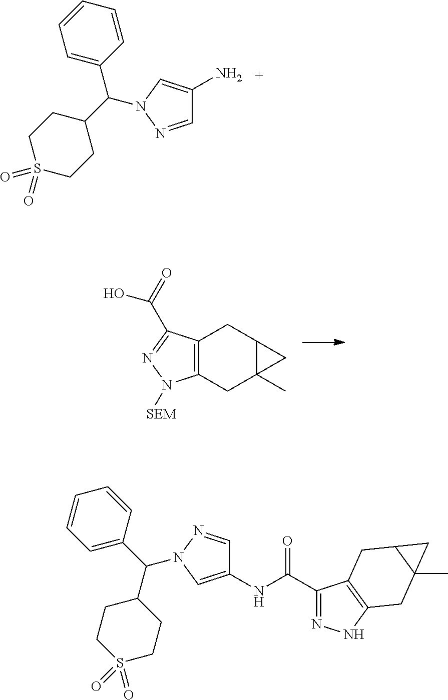 [SMILES: Nc1cnn(C(c2ccccc2)C2CCS(=O)(=O)CC2)c1 ; CC12Cc3c(c(C(=O)O)nn3COCC[Si](C)(C)C)CC1C2 ; CC12Cc3[nH]nc(C(=O)Nc4cnn(C(c5ccccc5)C5CCS(=O)(=O)CC5)c4)c3CC1C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5a-methyl-1-((2-(trimethylsilyl)ethoxy)methyl)-1,4,4a,5,5a,6-hexahydrocyclopropa[f]indazole-3-carboxylic acid (Example C31a) and 3-((4-amino-1H-pyrazol-1-yl)methyl)benzonitrile (Example A1) with 4-((4-amino-1H-pyrazol-1-yl)(phenyl)methyl)tetrahydro-2H-thiopyran 1,1-dioxide (Example A55). Also, the stereoisomers were separated by preparative chiral HPLC instead of SFC.

Chiral HPLC Conditions: ChiralPak IB-3 (4.6×50 mm, 3 μm particle size); eluent=Hex (0.1% Et₃N):EtOH 60:40; 1.0 ml/min, 3.0 MPA, 25° C.

83a: ¹H NMR (300 MHz, CDCl₃) δ 8.70 (s, 1H), 8.13 (s, 1H), 7.57 (s, 1H), 7.40-7.43 (m, 2H), 7.29-7.36 (m, 3H), 4.84 (d, 1H, J=10.8 Hz), 3.38 (d, 1H, J=16.8 Hz), 2.86-3.05 (m, 6H), 2.68-2.79 (m, 2H), 1.80-1.89 (m, 4H), 1.24 (s, 3H), 1.06-1.12 (m, 1H), 0.41-0.42 (m, 1H), 0.13-0.23 (m, 1H); MS: m/z=480 (M+H); HPLC retention time: 2.40 min.

83b: ¹H NMR (300 MHz, CDCl₃) δ 8.63 (s, 1H), 8.14 (s, 1H), 7.57 (s, 1H), 7.28-7.45 (m, 5H), 4.84 (d, 1H, J=10.5 Hz), 3.46 (d, 1H, J=30.0 Hz), 2.71-3.5 (m, 8H), 1.81-1.97 (m, 4H), 1.26 (s, 3H), 0.90-1.14 (m, 1H), 041-0.42 (m, 1H), 0.13-0.23 (m, 1H); MS: m/z=480 (M+H); HPLC retention time: 3.94 min.

### Examples 84a and 84b

### 6,6-dimethyl-N-(1-(1-(thiazol-4-yl)ethyl)-1H-pyrazol-4-yl)-4,5,6,7-tetrahydro-1H-indazole-3-carboxamide

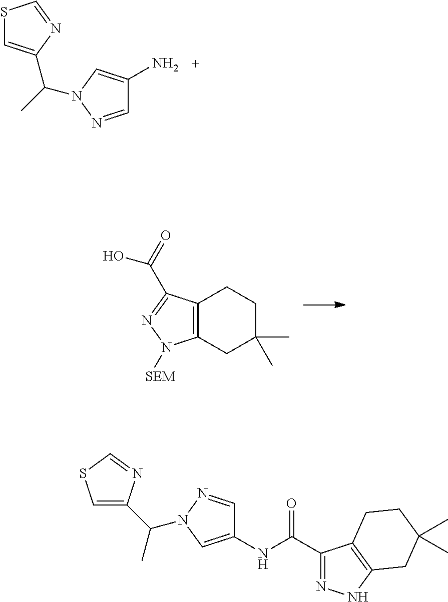 [SMILES: CC(c1cscn1)n1cc(N)cn1 ; CC1(C)CCc2c(C(=O)O)nn(COCC[Si](C)(C)C)c2C1 ; CC(c1cscn1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(thiazol-4-yl)ethyl)-1H-pyrazol-4-amine (Example A33).

SFC conditions: Chiralpak IA (4.6×50 mm, 5 μm particle size) at 55% methanol w/0.1% NH₄OH; 4 mL/min, 120 bars, 40° C.

84a: ¹H NMR (400 MHz, DMSO-d₆) δ 12.78 (s, 1H), 10.05 (s, 1H), 9.07 (d, J=1.9 Hz, 1H), 8.04 (d, J=0.7 Hz, 1H), 7.64 (s, 1H), 7.48 (dd, J=2.0, 0.7 Hz, 1H), 5.72 (q, J=7.0 Hz, 1H), 2.66 (t, J=6.3 Hz, 2H), 2.38 (s, 3H), 1.81 (d, J=7.0 Hz, 3H), 1.47 (t, J=6.4 Hz, 2H), 0.96 (s, 7H); MS: m/z=371 (M+H); SFC retention time: 0.53 min.

84b: ¹H NMR (400 MHz, DMSO-d₆) δ 12.78 (s, 1H), 10.05 (s, 1H), 9.07 (d, J=1.9 Hz, 1H), 8.04 (d, J=0.7 Hz, 1H), 7.64 (s, 1H), 7.48 (dd, J=2.0, 0.7 Hz, 1H), 5.72 (d, J=7.0 Hz, 1H), 2.66 (t, J=6.3 Hz, 2H), 2.38 (s, 3H), 1.81 (d, J=7.0 Hz, 4H), 1.47 (t, J=6.4 Hz, 2H), 0.96 (s, 7H; MS: m/z=371 (M+H); SFC retention time: 0.92 min.

### Examples 85a and 85b

### 6,6-dimethyl-N-(1-((tetrahydro-2H-thiopyran-4-yl)(thiazol-2-yl)methyl)-1H-pyrazol-4-yl)-4,5,6,7-tetrahydro-1H-indazole-3-carboxamide

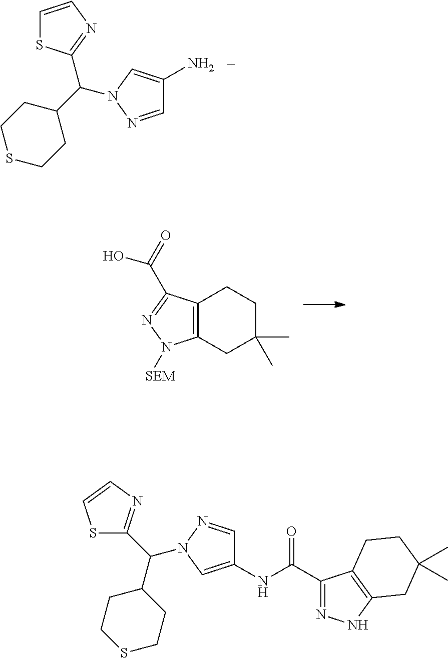 [SMILES: Nc1cnn(C(c2nccs2)C2CCSCC2)c1 ; CC1(C)CCc2c(C(=O)O)nn(COCC[Si](C)(C)C)c2C1 ; CC1(C)CCc2c(C(=O)Nc3cnn(C(c4nccs4)C4CCSCC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-((tetrahydro-2H-thiopyran-4-yl)(thiazol-2-yl)methyl)-1H-pyrazol-4-amine (Example A18).

SFC conditions: Lux Cellulose-3 (4.6×50 mm, 5 μm particle size) at 20% methanol w/0.1% NH₄OH; 4 mL/min, 120 bars, 40° C.

85a: ¹H NMR (400 MHz, DMSO-d₆) δ 12.87-12.66 (s, 1H), 10.21-10.01 (s, 1H), 8.24-8.20 (s, 1H), 7.81-7.77 (d, J=3.3 Hz, 1H), 7.74-7.72 (s, 1H), 7.72-7.70 (d, J=3.2 Hz, 1H), 5.77-5.58 (d, J=9.9 Hz, 1H), 2.72-2.63 (m, 2H), 2.61-2.52 (m, 4H), 2.40-2.37 (s, 2H), 1.77-1.67 (m, 1H), 1.52-1.27 (m, 6H), 0.99-0.94 (s, 6H); MS: m/z=457 (M+H); SFC retention time: 0.70 min.

85b: ¹H NMR (400 MHz, DMSO-d₆) δ 12.87-12.66 (s, 1H), 10.21-10.01 (s, 1H), 8.24-8.20 (s, 1H), 7.81-7.77 (d, J=3.3 Hz, 1H), 7.74-7.72 (s, 1H), 7.72-7.70 (d, J=3.2 Hz, 1H), 5.77-5.58 (d, J=9.9 Hz, 1H), 2.72-2.63 (m, 2H), 2.61-2.52 (m, 4H), 2.40-2.37 (s, 2H), 1.77-1.67 (m, 1H), 1.52-1.27 (m, 6H), 0.99-0.94 (s, 6H); MS: m/z=457 (M+H); SFC retention time: 0.58 min.

### Examples 86a-d

### 6,6-dimethyl-N-(1-((1-oxidotetrahydro-2H-thiopyran-4-yl)(phenyl)methyl)-1H-pyrazol-4-yl)-4,5,6,7-tetrahydro-1H-indazole-3-carboxamide

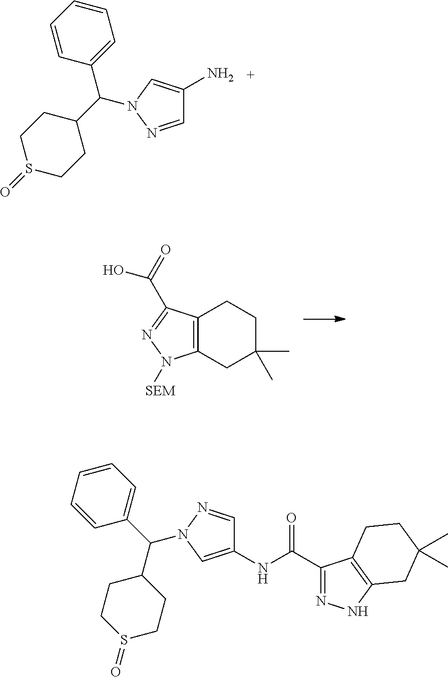 [SMILES: Nc1cnn(C(c2ccccc2)C2CCS(=O)CC2)c1 ; CC1(C)CCc2c(C(=O)O)nn(COCC[Si](C)(C)C)c2C1 ; CC1(C)CCc2c(C(=O)Nc3cnn(C(c4ccccc4)C4CCS(=O)CC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 4-((4-amino-1H-pyrazol-1-yl)(phenyl)methyl)tetrahydro-2H-thiopyran 1-oxide (Example A66). Each diastereomer of A66 is reacted independently, then resolved into their constituent stereoisomers by chiral LCMS yielding the 4 diastereomeric title products.

Chiral HPLC Conditions (86a/b): ChiralPak IA-3 (4.6×50 mm, 3 μm particle size); eluent=Hex (0.1% Et₃N):EtOH 50:50; 1.0 ml/min, 5.8 MPA, 25° C.

86a: ¹H NMR (300 MHz, CDCl₃) δ 8.85 (s, 1H), 8.14 (s, 1H), 7.56 (s, 1H), 7.41 (d, J=1.5 Hz, 2H), 7.39-7.26 (m, 3H), 4.96 (d, J=10.8 Hz, 1H), 3.03-2.94 (m, 2H), 2.85 (t, J=6.3 Hz, 2H), 2.72-2.60 (m, 1H), 2.52-2.34 (m, 4H), 2.27-1.97 (m, 2H), 1.56 (t, J=6.4 Hz, 2H), 1.49-1.38 (m, 2H), 1.00 (s, 6H); MS: m/z=466 (M+H); HPLC retention time: 2.71 min.

86b: ¹H NMR (300 MHz, CDCl₃) δ 8.84 (s, 1H), 8.14 (s, 1H), 7.56 (s, 1H), 7.41 (d, J=1.5 Hz, 2H), 7.39-7.26 (m, 3H), 4.96 (d, J=10.8 Hz, 1H), 3.03-2.93 (m, 2H), 2.85 (t, J=6.3 Hz, 2H), 2.72-2.60 (m, 1H), 2.51-2.34 (m, 4H), 2.27-1.97 (m, 2H), 1.56 (t, J=6.3 Hz, 2H), 1.48-1.38 (m, 2H), 1.00 (s, 6H); MS: m/z=466 (M+H); HPLC retention time: 5.89 min.

Chiral HPLC Conditions (86c/d): ChiralPak IB (4.6×250 mm, 5 μm particle size); eluent=Hex (0.1% Et₃N):EtOH 70:30; 1.0 ml/min, 5.9 MPA, 25° C.

86c: ¹H NMR (300 MHz, CDCl₃) δ 8.69 (s, 1H), 8.16 (s, 1H), 7.52 (s, 1H), 7.42-7.28 (m, 5H), 4.76 (d, J=10.8 Hz, 1H), 3.32-3.22 (m, 2H), 2.83 (t, J=6.3 Hz, 2H), 2.77-2.58 (m, 3H), 2.40 (s, 2H), 1.89-1.81 (m, 2H), 1.55 (t, J=6.3 Hz, 2H), 1.48-1.35 (m, 2H), 1.00 (s, 6H); MS: m/z=466 (M+H); HPLC retention time: 9.74 min.

86d: ¹H NMR (300 MHz, CDCl₃) δ 8.66 (s, 1H), 8.15 (s, 1H), 7.53 (s, 1H), 7.42-7.29 (m, 5H), 4.77 (d, J=10.8 Hz, 1H), 3.33-3.23 (m, 2H), 2.84 (t, J=6.3 Hz, 2H), 2.78-2.59 (m, 3H), 2.41 (s, 2H), 1.87-1.82 (m, 2H), 1.56 (t, J=6.4 Hz, 2H), 1.50-1.32 (m, 2H), 1.00 (s, 6H); MS: m/z=466 (M+H); HPLC retention time: 15.63 min.

### Examples 87a and 87b

### N-(1-((1,1-dioxidotetrahydro-2H-thiopyran-4-yl)(thiazol-2-yl)methyl)-1H-pyrazol-4-yl)-6,6-dimethyl-4,5,6,7-tetrahydro-1H-indazole-3-carboxamide

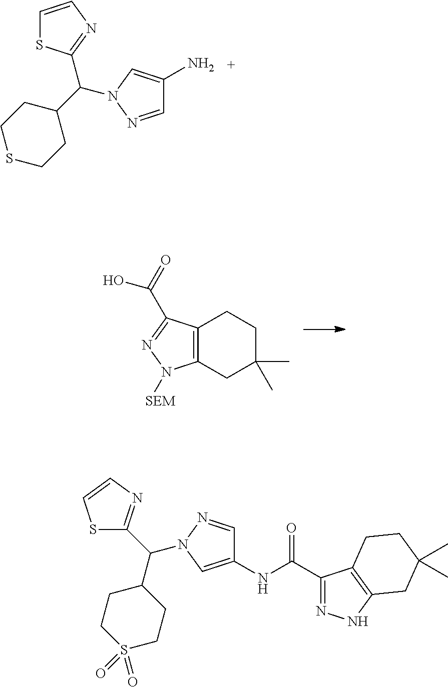 [SMILES: Nc1cnn(C(c2nccs2)C2CCSCC2)c1 ; CC1(C)CCc2c(C(=O)O)nn(COCC[Si](C)(C)C)c2C1 ; CC1(C)CCc2c(C(=O)Nc3cnn(C(c4nccs4)C4CCS(=O)(=O)CC4)c3)n[nH]c2C1]

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with 1-((tetrahydro-2H-thiopyran-4-yl)(thiazol-2-yl)methyl)-1H-pyrazol-4-amine (Example A18).

SFC conditions: Chiralpak IC (4.6×50 mm, 5 μm particle size) at 50% methanol w/0.10% NH₄OH; 4 mL/min, 120 bars, 40° C.

87a: ¹H NMR (400 MHz, DMSO-d₆) δ 12.93-12.70 (s, 1H), 10.31-10.08 (s, 1H), 8.29-8.27 (d, J=0.7 Hz, 1H), 7.83-7.80 (d, J=3.3 Hz, 1H), 7.78-7.76 (s, 1H), 7.75-7.72 (d, J=3.2 Hz, 1H), 6.04-5.80 (d, J=9.8 Hz, 1H), 3.21-2.97 (m, 4H), 2.84-2.71 (m, 1H), 2.70-2.62 (t, J=6.3 Hz, 2H), 2.41-2.36 (s, 2H), 1.92-1.64 (m, 3H), 1.62-1.50 (m, 1H), 1.50-1.44 (t, J=6.3 Hz, 2H), 0.98-0.94 (s, 6H); MS: m/z=489 (M+H); SFC retention time: 0.61 min.

87b: ¹H NMR (400 MHz, DMSO-d₆) δ 12.93-12.70 (s, 1H), 10.31-10.08 (s, 1H), 8.29-8.27 (d, J=0.7 Hz, 1H), 7.83-7.80 (d, J=3.3 Hz, 1H), 7.78-7.76 (s, 1H), 7.75-7.72 (d, J=3.2 Hz, 1H), 6.04-5.80 (d, J=9.8 Hz, 1H), 3.21-2.97 (m, 4H), 2.84-2.71 (m, 1H), 2.70-2.62 (t, J=6.3 Hz, 2H), 2.41-2.36 (s, 2H), 1.92-1.64 (m, 3H), 1.62-1.50 (m, 1H), 1.50-1.44 (t, J=6.3 Hz, 2H), 0.98-0.94 (s, 6H); MS: m/z=489 (M+H); SFC retention time: 0.80 min.

### Examples 88a and 88b

### 6,6-dimethyl-N-(1-(pyridin-3-yl(tetrahydro-2H-pyran-4-yl)methyl)-1H-pyrazol-4-yl)-4,5,6,7-tetrahydro-1H-indazole-3-carboxamide

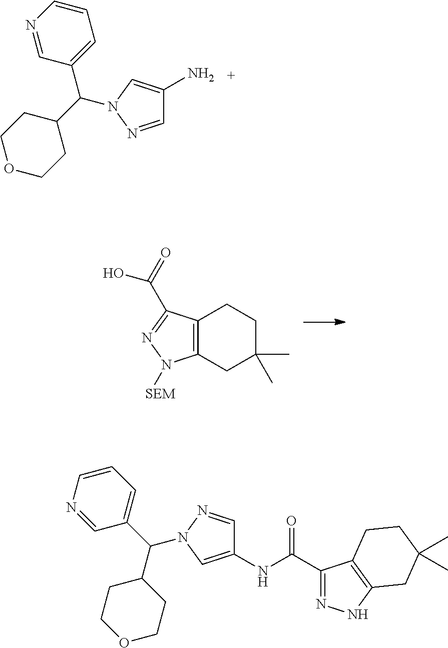 [SMILES: Nc1cnn(C(c2cccnc2)C2CCOCC2)c1 ; CC1(C)CCc2c(C(=O)O)nn(COCC[Si](C)(C)C)c2C1 ; CC1(C)CCc2c(C(=O)Nc3cnn(C(c4cccnc4)C4CCOCC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(pyridin-3-yl(tetrahydro-2H-pyran-4-yl)methyl)-1H-pyrazol-4-amine (Example A19).

SFC conditions: Chiralpak IC (4.6×50 mm, 5 μm particle size) at 35% methanol w/0.10% NH₄OH; 4 mL/min, 120 bars, 40° C.

88a: ¹H NMR (400 MHz, DMSO-d₆) δ 12.78 (s, 1H), 10.06 (s, 1H), 8.71 (dd, J=2.3, 0.8 Hz, 1H), 8.50 (dd, J=4.8, 1.6 Hz, 1H), 8.19 (d, J=0.7 Hz, 1H), 7.98 (dt, J=8.0, 1.9 Hz, 1H), 7.68 (s, 1H), 7.38 (ddd, J=8.0, 4.8, 0.8 Hz, 1H), 5.16 (d, J=10.8 Hz, 1H), 3.80 (t, J=12.9 Hz, 2H), 3.28-3.21 (m, 2H), 2.79-2.57 (m, 3H), 2.38 (s, 2H), 1.47 (t, J=6.4 Hz, 2H), 1.34-1.00 (m, 4H), 0.96 (s, 6H); MS: m/z=435 (M+H); SFC retention time: 0.96 min.

88b: ¹H NMR (400 MHz, DMSO-d₆) δ 12.78 (s, 1H), 10.06 (s, 1H), 8.71 (dd, J=2.3, 0.8 Hz, 1H), 8.50 (dd, J=4.8, 1.6 Hz, 1H), 8.19 (s, 1H), 7.98 (dt, J=7.9, 2.0 Hz, 1H), 7.68 (s, 1H), 7.43-7.33 (m, 1H), 5.16 (d, J=10.8 Hz, 1H), 3.80 (t, J=13.0 Hz, 2H), 3.27-3.19 (m, 1H), 2.66 (t, J=6.5 Hz, 3H), 2.38 (s, 2H), 1.47 (t, J=6.4 Hz, 2H), 1.35-1.02 (m, 4H), 0.96 (s, 5H); MS: m/z=435 (M+H); SFC retention time: 1.05 min.

### Examples 89a and 89b

### N-(1-((1,1-dioxidothietan-3-yl)(phenyl)methyl)-1H-pyrazol-4-yl)-6,6-dimethyl-4,5,6,7-tetrahydro-1H-indazole-3-carboxamide

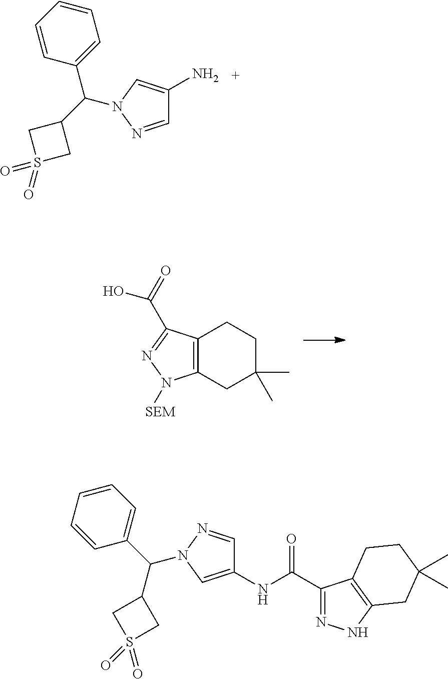 [SMILES: Nc1cnn(C(c2ccccc2)C2CS(=O)(=O)C2)c1 ; CC1(C)CCc2c(C(=O)O)nn(COCC[Si](C)(C)C)c2C1 ; CC1(C)CCc2c(C(=O)Nc3cnn(C(c4ccccc4)C4CS(=O)(=O)C4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 3-((4-amino-1H-pyrazol-1-yl)(phenyl)methyl)thietane 1,1-dioxide (Example A60). Also, the stereoisomers were separated by preparative chiral HPLC instead of SFC.

Chiral HPLC Conditions: ChiralPak IB-3 (4.6×50 mm, 3 μm particle size); eluent=Hex (0.1% Et₃N):EtOH 60:40; 1.0 ml/min, 3.5 MPA, 25° C.

89a: ¹H NMR (300 MHz, DMSO-d₆): δ 10.15 (s, 1H), 8.15 (s, 1H), 7.68 (s, 1H), 7.47-7.49 (m, 2H), 7.32-7.39 (m, 3H), 5.66 (d, 1H, J=10.2), 4.06-4.19 (m, 2H), 3.72-3.93 (m, 3H), 2.63-2.67 (m, 2H), 2.38 (s, 2H), 1.99 (m, 2H), 0.96 (s, 6H); MS: m/z=454 (M+H); HPLC retention time: 1.98 min.

89b: ¹H NMR (300 MHz, CDCl₃) δ 10.14 (s, 1H), 8.15 (s, 1H), 7.67 (s, 1H), 7.461-7.484 (m, 2H), 7.31-7.38 (m, 3H), 5.65 (d, 1H, J=10.8), 4.06-4.18 (m, 2H), 3.71-3.92 (m, 3H), 2.60-2.70 (m, 2H), 2.35 (s, 2H), 1.44-1.48 (m, 2H), 0.95 (s, 6H); MS: m/z=454 (M+H); HPLC retention time: 4.04 min.

### Examples 90a and 90b

### N-(1-benzyl-1H-pyrazol-4-yl)-5-cyano-6,6-dimethyl-4,5,6,7-tetrahydro-1H-indazole-3-carboxamide

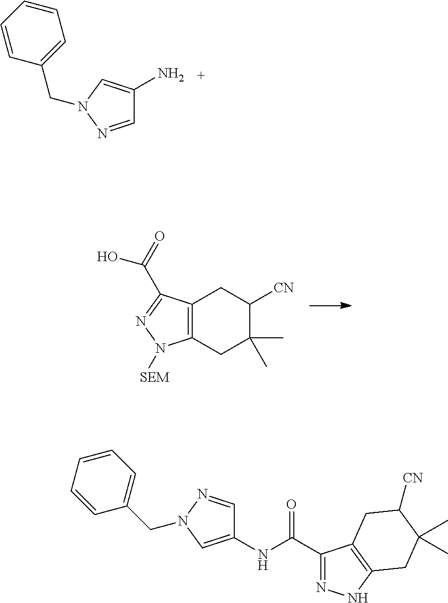 [SMILES: Nc1cnn(Cc2ccccc2)c1 ; CC1(C)Cc2c(c(C(=O)O)nn2COCC[Si](C)(C)C)CC1C#N ; CC1(C)Cc2[nH]nc(C(=O)Nc3cnn(Cc4ccccc4)c3)c2CC1C#N]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5-cyano-6,6-dimethyl-1-((2-(trimethylsilyl)ethoxy)methyl)-4,5,6,7-tetrahydro-1H-indazole-3-carb oxylic acid (Example C27) and 3-((4-amino-1H-pyrazol-1-yl)methyl)benzonitrile (Example A1) with 1-benzyl-1H-pyrazol-4-amine (Example A2).

SFC conditions: Lux Cellulose-4 (4.6×50 mm, 5 μm particle size) at 35% methanol w/0.1% NH₄OH; 4 mL/min, 120 bars, 40° C.

90a: ¹H NMR (400 MHz, DMSO-d₆) δ 13.02 (s, 1H), 10.21 (s, 1H), 8.08 (s, 1H), 7.65 (d, J=0.5 Hz, 1H), 7.37-7.21 (m, 5H), 5.28 (s, 2H), 3.13-3.01 (m, 2H), 2.91 (dd, J=15.8, 6.3 Hz, 1H), 2.57 (s, 2H), 1.10 (s, 3H), 1.08 (s, 3H); MS: m/z=375 (M+H); SFC retention time: 0.49 min.

90b: ¹H NMR (400 MHz, DMSO-d₆) δ 13.02 (s, 1H), 10.21 (s, 1H), 8.08 (s, 1H), 7.65 (s, 1H), 7.37-7.20 (m, 5H), 5.28 (s, 2H), 3.13-3.00 (m, 2H), 2.91 (dd, J=15.7, 6.2 Hz, 1H), 2.57 (s, 2H), 1.10 (s, 3H), 1.08 (s, 3H); MS: m/z=375 (M+H); SFC retention time: 0.69 min.

### Examples 91a and 91b

### 6,6-dimethyl-N-(1-(1-(1-methyl-1H-pyrazol-4-yl)ethyl)-1H-pyrazol-4-yl)-4,5,6,7-tetrahydro-1H-indazole-3-carboxamide

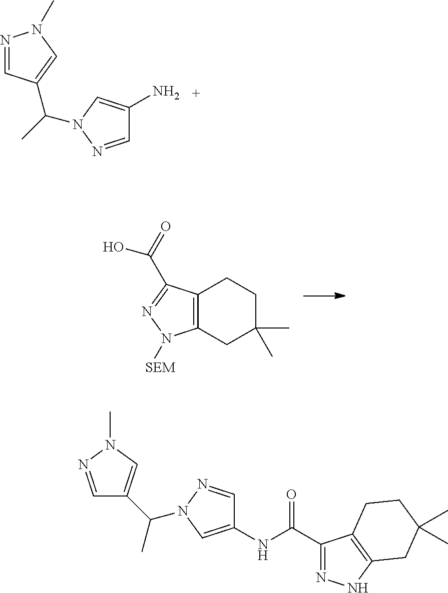 [SMILES: CC(c1cnn(C)c1)n1cc(N)cn1 ; CC1(C)CCc2c(C(=O)O)nn(COCC[Si](C)(C)C)c2C1 ; CC(c1cnn(C)c1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(1-methyl-1H-pyrazol-4-yl)ethyl)-1H-pyrazol-4-amine (Example A36).

SFC conditions: Chiralpak AD (4.6×50 mm, 5 μm particle size) at 55% methanol w/0.1% NH₄OH; 4 mL/min, 120 bars, 40° C.

91a: ¹H NMR (400 MHz, DMSO-d₆) δ 12.77 (s, 1H), 10.01 (s, 1H), 7.94 (d, J=0.6 Hz, 1H), 7.64 (s, 1H), 7.61 (d, J=0.7 Hz, 1H), 7.35 (d, J=0.9 Hz, 1H), 5.46 (q, J=6.9 Hz, 1H), 3.79 (s, 3H), 2.70-2.61 (m, 2H), 2.38 (s, 2H), 1.70 (d, J=6.9 Hz, 3H), 1.46 (t, J=6.4 Hz, 2H), 0.96 (s, 6H); MS: m/z=368 (M+H); SFC retention time: 0.41 min.

91b: ¹H NMR (400 MHz, DMSO-d₆) δ 12.77 (s, 1H), 10.01 (s, 1H), 7.94 (s, 1H), 7.64 (s, 1H), 7.61 (s, 1H), 7.35 (s, 1H), 5.46 (q, J=6.9 Hz, 1H), 3.79 (s, 3H), 2.73-2.59 (m, 2H), 2.38 (s, 2H), 1.70 (d, J=7.0 Hz, 3H), 1.46 (t, J=6.4 Hz, 2H), 0.96 (s, 6H); MS: m/z=368 (M+H); SFC retention time: 0.32 min.

### Examples 92a and 92b

### 6,6-dimethyl-N-(1-(1-(thiazol-2-yl)ethyl)-1H-pyrazol-4-yl)-4,5,6,7-tetrahydro-1H-indazole-3-carboxamide

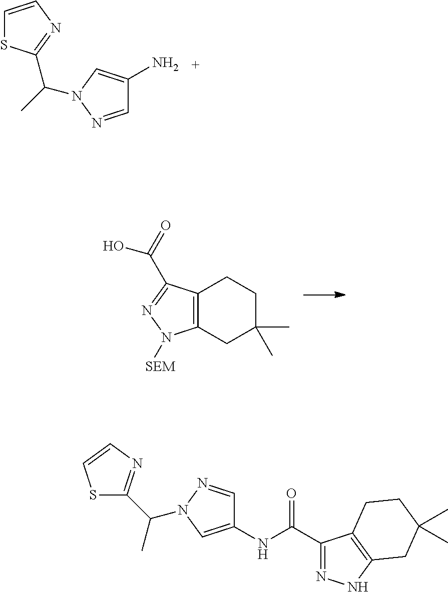 [SMILES: CC(c1nccs1)n1cc(N)cn1 ; CC1(C)CCc2c(C(=O)O)nn(COCC[Si](C)(C)C)c2C1 ; CC(c1nccs1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(thiazol-2-yl)ethyl)-1H-pyrazol-4-amine (Example A39).

SFC conditions: Chiralpak AD (4.6×50 mm, 5 μm particle size) at 55% methanol w/0.1% NH₄OH; 4 mL/min, 120 bars, 40° C.

92a: ¹H NMR (400 MHz, DMSO-d₆) δ 12.80 (s, 1H), 10.13 (s, 1H), 8.17 (d, J=0.5 Hz, 1H), 7.76 (d, J=3.3 Hz, 1H), 7.73 (s, 1H), 7.66 (d, J=3.3 Hz, 1H), 5.96 (q, J=7.0 Hz, 1H), 2.67 (t, J=6.2 Hz, 2H), 2.39 (s, 2H), 1.88 (d, J=7.0 Hz, 3H), 1.47 (t, J=6.4 Hz, 2H), 0.97 (s, 7H); MS: m/z=371 (M+H); SFC retention time: 0.67 min.

92b: ¹H NMR (400 MHz, DMSO-d₆) δ 12.80 (s, 1H), 10.13 (s, 1H), 8.17 (s, 1H), 7.76 (d, J=3.5 Hz, 1H), 7.72 (s, 1H), 7.66 (d, J=3.5 Hz, 1H), 5.96 (q, J=7.0 Hz, 1H), 3.27 (s, 1H), 2.67 (t, J=6.3 Hz, 2H), 2.39 (s, 2H), 1.88 (d, J=7.0 Hz, 3H), 1.47 (t, J=6.3 Hz, 2H), 0.97 (s, 6H); MS: m/z=371 (M+H); SFC retention time: 0.47 min.

### Examples 93a and 93b

### 5a-methyl-N-(1-(2-(methylsulfonyl)-1-(pyridin-3-yl)ethyl)-1H-pyrazol-4-yl)-1,4,4a,5,5a,6-hexahydrocyclopropa[f]indazole-3-carboxamide

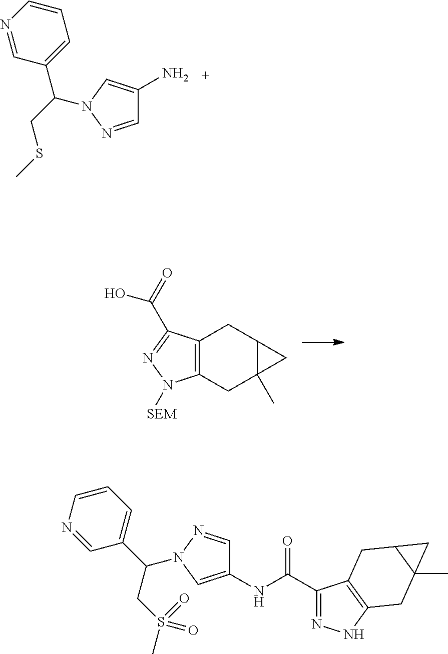 [SMILES: CSCC(c1cccnc1)n1cc(N)cn1 ; CC12Cc3c(c(C(=O)O)nn3COCC[Si](C)(C)C)CC1C2 ; CC12Cc3[nH]nc(C(=O)Nc4cnn(C(CS(C)(=O)=O)c5cccnc5)c4)c3CC1C2]

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with 1-(2-(methylthio)-1-(pyridin-3-yl)ethyl)-1H-pyrazol-4-amine (Example A26) and 6,6-dimethyl-1-((2-(trimethylsilyl)ethoxy)methyl)-4,5,6,7-tetrahydro-1H-indazole-3-carboxylic acid (Example C6) with 5a-methyl-1-((2-(trimethylsilyl)ethoxy)methyl)-1,4,4a,5,5a,6-hexahydrocyclopropa[f]indazole-3-carboxylic acid (Example C31a). It is important to note that the mCPBA oxidation needs to be monitored closely by LCMS to prevent over-oxidation to the pyridine N-oxide (generally <15 min. reaction time). Also, the stereoisomers were separated by preparative chiral HPLC instead of SFC.

Chiral HPLC Conditions: ChiralPak IC-3 (4.6×50 mm, 3 μm particle size); eluent=Hex (0.1% Et₃N):EtOH 50:50; 1.0 ml/min, 4.4 MPA, 25° C.

93a: ¹H NMR (300 HMz, CD₃OD) δ 8.45 (s, 1H), 8.32-8.33 (m, 1H), 8.06 (s, 1H), 7.76-7.79 (m, 1H), 7.61 (s, 1H), 7.25-7.28 (m, 1H), 5.92-5.97 (m, 1H), 4.42-4.69 (m, 1H), 3.65-3.72 (m, 1H), 3.06 (s, 1H), 2.73-2.89 (m, 2H), 2.44-2.55 (m, 4H), 1.07 (s, 3H), 0.88-0.94 (m, 1H), 0.18-0.23 (m, 1H), 0.00-0.02 (m, 1H); MS: m/z=441 (M+H); HPLC retention time: 4.40 min.

93b: ¹H NMR (300 HMz, CD₃OD) δ 8.46 (s, 1H), 8.32-8.34 (m, 1H), 8.08 (s, 1H), 7.78-7.80 (m, 1H), 7.62 (s, 1H), 7.25-7.29 (m, 1H), 5.93-5.97 (m, 1H), 4.43-4.69 (m, 1H), 3.66-3.73 (m, 1H), 3.06 (s, 1H), 2.75-2.90 (m, 2H), 2.45-2.56 (m, 4H), 1.08 (s, 3H), 0.88-0.94 (m, 1H), 0.18-0.23 (m, 1H), −0.02-0.02 (m, 1H); MS: m/z=441 (M+H); HPLC retention time: 6.15 min.

### Examples 94a and 94b

### N-(1-((1,1-dioxidotetrahydro-2H-thiopyran-4-yl)(phenyl)methyl)-1H-pyrazol-4-yl)-5,5-dimethyl-1,4,5,6-tetrahydrocyclopenta[c]n pyrazole-3-carboxamide PP-277, 1

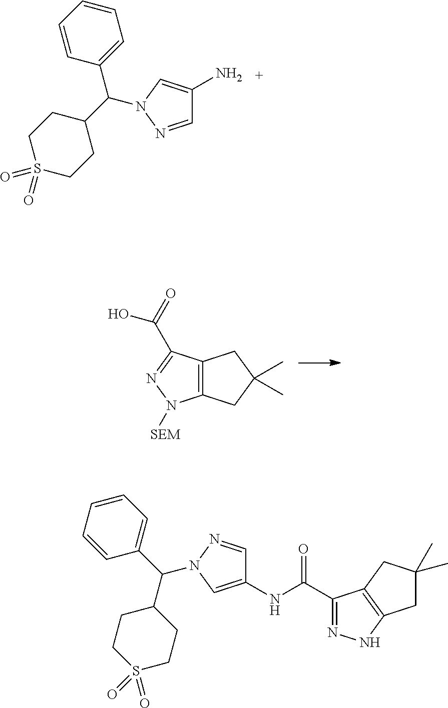 [SMILES: Nc1cnn(C(c2ccccc2)C2CCS(=O)(=O)CC2)c1 ; CC1(C)Cc2c(C(=O)O)nn(COCC[Si](C)(C)C)c2C1 ; CC1(C)Cc2[nH]nc(C(=O)Nc3cnn(C(c4ccccc4)C4CCS(=O)(=O)CC4)c3)c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5,5-dimethyl-1-((2-(trimethylsilyl)ethoxy)methyl)-1,4,5,6-tetrahydrocyclopenta[c]pyrazole-3-carboxylic acid (Example C18) and 3-((4-amino-1H-pyrazol-1-yl)methyl)benzonitrile (Example A1) with 4-((4-amino-1H-pyrazol-1-yl)(phenyl)methyl)tetrahydro-2H-thiopyran 1,1-dioxide (Example A55). Also, the stereoisomers were separated by preparative chiral HPLC instead of SFC.

Chiral HPLC Conditions: ChiralPak IB-3 (4.6×50 mm, 3 μm particle size); eluent=Hex (0.1% Et₃N):EtOH 65:35; 1.0 ml/min, 4.2 MPA, 25° C.

94a: ¹H NMR (300 HMz, CD₃OD) δ 8.16 (s, 1H), 7.70 (s, 1H), 7.55-7.57 (m, 2H), 7.30-7.41 (m, 3H), 5.12-5.16 (d, 1H, J=11.1), 3.02-3.34 (m, 5H), 2.84-2.97 (m, 1H), 2.59-2.87 (s, 4H), 1.92-1.69 (m, 1H), 1.75-1.85 (m, 3H), 1.24 (s, 6H); MS: m/z=468 (M+H); HPLC retention time: 2.54 min.

94b: ¹H NMR (300 HMz, CD₃OD) δ 8.04 (s, 1H), 7.59 (s, 1H), 7.43-7.46 (m, 2H), 7.18-7.29 (m, 3H), 5.00-5.04 (d, 1H, J=10.8), 2.83-3.09 (m, 4H), 2.73-2.79 (m, 1H), 2.47-2.54 (m, 4H), 1.76-1.80 (m, 1H), 1.60-1.76 (m, 3H), 1.15 (s, 6H); MS: m/z=468 (M+H); HPLC retention time: 3.67 min.

### Examples 95a and 95b

### N-(1-benzyl-1H-pyrazol-4-yl)-5-(hydroxymethyl)-6,6-dimethyl-4,5,6,7-tetrahydro-1H-indazole-3-carboxamide

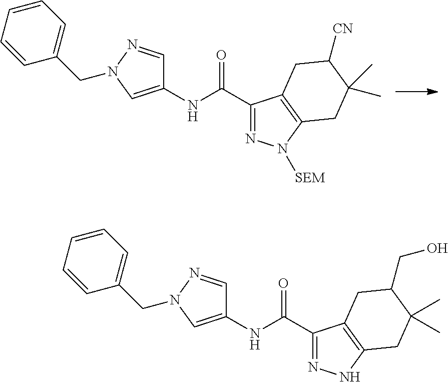 [SMILES: CC1(C)Cc2c(c(C(=O)Nc3cnn(Cc4ccccc4)c3)nn2COCC[Si](C)(C)C)CC1C#N ; CC1(C)Cc2[nH]nc(C(=O)Nc3cnn(Cc4ccccc4)c3)c2CC1CO]

N-(1-benzylpyrazol-4-yl)-5-cyano-6,6-dimethyl-1-(2-trimethylsilylethoxymethyl)-5,7-dihydro-4H-indazole-3-carboxamide (0.302 g, 0.599 mmol, intermediate towards Examples 90a/b) in dry dichloromethane (3.5 mL) at −78° C. was added 1.0M diisobutylaluminum hydride in dichloromethane (6.0 equiv., 3.59 mmol, 3.6 mL) dropwise. The sample was stirred at −78° C. for 1 hour. MeOH (5 mL) was added, then the mixture was diluted with NH₄Cl and H₂O, filtered through a pad of Celite, extracted 6 times with 10% MeOH in dichloromethane, dried over MgSO₄, filtered, and chromatographed through silica gel (12 g, 0-100% EtOAc in heptane, 11 min gradient, Rf product ˜0.3 in 1:1 heptane:EtOAc) to provide N-(1-benzylpyrazol-4-yl)-5-formyl-6,6-dimethyl-1-(2-trimethylsilylethoxymethyl)-5,7-dihydro-4H-indazole-3-carboxamide (118 mg, 0.233 mmol, 39% yield).

To a solution of N-(1-benzylpyrazol-4-yl)-5-formyl-6,6-dimethyl-1-(2-trimethylsilylethoxymethyl)-5,7-dihydro-4H-indazole-3-carboxamide (0.136 g, 0.268 mmol) in dry ethanol (3 mL) at 0° C. was added sodium borohydride (2.00 equiv., 0.535 mmol, 21 mg) slowly. The sample was warmed to room temperature and additional EtOH (3 mL) was added to help solubilize the reaction. The sample was stirred for 1 hour. The sample was quenched by adding sat. NaHCO₃, extracted 9 times with 10% MeOH in dichloromethane, dried over MgSO₄, filtered, and concentrated in vacuo. Purification by CombiFlash (4 g, 0-100% EtOAc in heptane, 14 min gradient) provided N-(1-benzyl-1H-pyrazol-4-yl)-5-(hydroxymethyl)-6,6-dimethyl-1-((2-(trimethylsilyl)ethoxy)methyl)-4,5,6,7-tetrahydro-1H-indazole-3-carboxamide (121 mg, 0.238 mmol, 89% yield).

This material was then dissolved in TFA and triisopropylsilane (5 equiv.) was added, plus a few drops CH₂Cl₂ to homogenize. The mixture was stirred for 90 minutes, then concentrated and purified by reverse phase HPLC followed by SFC on a chiral stationary phase to provide the title compounds as single enantiomers.

SFC conditions: Chiralpak IC (4.6×50 mm, 5 μm particle size) at 40% ethanol w/0.1% NH₄OH; 4 mL/min, 120 bars, 40° C.

95a: ¹H NMR (400 MHz, DMSO-d₆) δ 12.76 (s, 1H), 10.06 (s, 1H), 8.06 (s, 1H), 7.65 (s, 1H), 7.37-7.20 (m, 5H), 5.27 (s, 2H), 4.36 (t, J=5.1 Hz, 1H), 3.70-3.63 (m, 1H), 3.23-3.15 (m, 1H), 2.89 (dd, J=17.1, 5.3 Hz, 1H), 2.47-2.29 (m, 3H), 1.58-1.49 (m, 1H), 1.02 (s, 3H), 0.86 (s, 3H); MS: m/z=380 (M+H); SFC retention time: 0.54 min.

95b: ¹H NMR (400 MHz, DMSO-d₆) δ 12.76 (s, 1H), 10.06 (s, 1H), 8.06 (s, 1H), 7.65 (d, J=0.6 Hz, 1H), 7.37-7.20 (m, 5H), 5.27 (s, 2H), 4.36 (t, J=5.1 Hz, 1H), 3.70-3.63 (m, 1H), 3.23-3.15 (m, 1H), 2.89 (dd, J=17.2, 5.4 Hz, 1H), 2.47-2.30 (m, 3H), 1.58-1.48 (m, 1H), 1.02 (s, 3H), 0.86 (s, 3H); MS: m/z=380 (M+H); SFC retention time: 0.67 min.

### Examples 96a and 96b

### N-(1-(2-(dimethylamino)-1-phenylethyl)-1H-pyrazol-4-yl)-5,5-dimethyl-1,4,5,6-tetrahydrocyclopenta[c]pyrazole-3-carboxamide

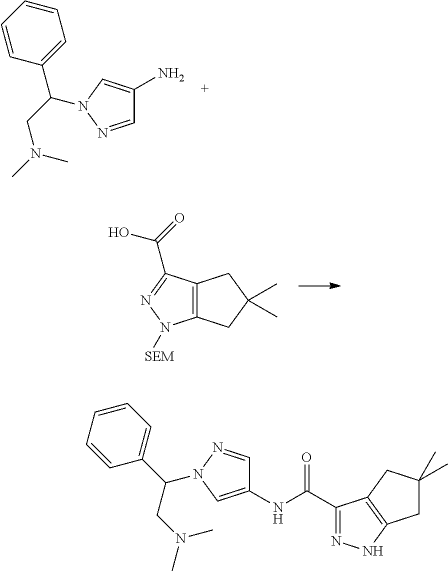 [SMILES: CN(C)CC(c1ccccc1)n1cc(N)cn1 ; CC1(C)Cc2c(C(=O)O)nn(COCC[Si](C)(C)C)c2C1 ; CN(C)CC(c1ccccc1)n1cc(NC(=O)c2n[nH]c3c2CC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5,5-dimethyl-1-((2-(trimethylsilyl)ethoxy)methyl)-1,4,5,6-tetrahydrocyclopenta[c]pyrazole-3-carboxylic acid (Example C18) and 3-((4-amino-1H-pyrazol-1-yl)methyl)benzonitrile (Example A1) with 1-(2-(dimethylamino)-1-phenylethyl)-1H-pyrazol-4-amine (Example A5). Also, the stereoisomers were separated by preparative chiral HPLC instead of SFC.

Chiral HPLC Conditions: Lux Cellulose-4 (4.6×150 mm, 3 μm particle size); eluent=Hex (0.1% Et₃N):IPA 75:25; 1.0 ml/min, 5.2 MPA, 25° C.

96a: ¹H NMR (300 HMz, CD₃OD) δ 8.17 (s, 1H), 7.72 (s, 1H), 7.28-7.33 (m, 5H), 5.57-5.61 (m, 1H), 3.46-3.54 (m, 1H), 2.86-2.93 (m, 1H), 2.59-2.66 (m, 4H), 2.25-2.31 (m, 6H), 1.16 (s, 6H); MS: m/z=393 (M+H); HPLC retention time: 7.13 min.

96b: ¹H NMR (300 HMz, CD₃OD) δ 8.17 (s, 1H), 7.72 (s, 1H), 7.28-7.37 (m, 5H), 5.57-5.62 (m, 1H), 3.46-3.54 (m, 1H), 2.87-2.93 (m, 1H), 2.59-2.66 (m, 4H), 2.31 (s, 6H), 1.26 (s, 6H); MS: m/z=393 (M+H); HPLC retention time: 11.74 min.

### Examples 97a and 97b

### 5,5-dimethyl-N-(1-(2-(methylsulfonyl)-1-(pyridin-3-yl)ethyl)-pyrazol-4-yl)-1,4,5,6-tetrahydrocyclopenta[c]pyrazole-3-carboxamide

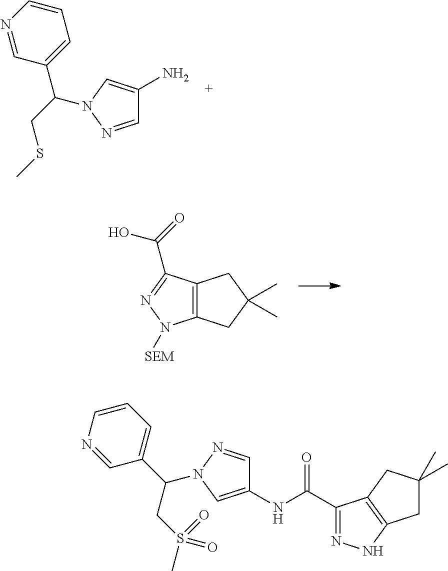 [SMILES: CSCC(c1cccnc1)n1cc(N)cn1 ; CC1(C)Cc2c(C(=O)O)nn(COCC[Si](C)(C)C)c2C1 ; CC1(C)Cc2[nH]nc(C(=O)Nc3cnn(C(CS(C)(=O)=O)c4cccnc4)c3)c2C1]

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with 1-(2-(methylthio)-1-(pyridin-3-yl)ethyl)-1H-pyrazol-4-amine (Example A26) and 6,6-dimethyl-1-((2-(trimethylsilyl)ethoxy)methyl)-4,5,6,7-tetrahydro-1H-indazole-3-carboxylic acid (Example C6) with 5,5-dimethyl-1-((2-(trimethylsilyl)ethoxy)methyl)-1,4,5,6-tetrahydrocyclopenta[c]pyrazole-3-carboxylic acid (Example C18). It is important to note that the mCPBA oxidation needs to be monitored closely by LCMS to prevent over-oxidation to the pyridine N-oxide (generally <15 min. reaction time). Also, the stereoisomers were separated by preparative chiral HPLC instead of SFC.

Chiral HPLC Conditions: ChiralPak IB-3 (4.6×50 mm, 3 μm particle size); eluent=Hex (0.1% Et₃N):EtOH 80:20; 1.0 ml/min, 2.2 MPA, 25° C.

97a: ¹H NMR (300 HMz, CD₃OD) 8.64-8.65 (m, 1H), 8.51-8.52 (m, 1H), 8.27 (s, 1H), 7.96-7.99 (m, 1H), 7.81 (s, 1H), 7.43-7.48 (m, 1H), 6.12-6.17 (m, 1H), 4.62-4.67 (m, 1H), 3.88-3.91 (m, 1H), 2.59-2.63 (m, 7H), 1.26 (s, 6H); MS: m/z=429 (M+H); HPLC retention time: 7.59 min.

97b: ¹H NMR (300 HMz, CD₃OD) δ 8.64-8.65 (m, 1H), 8.50-8.52 (m, 1H), 8.27 (s, 1H), 7.96-7.98 (m, 1H), 7.81 (s, 1H), 7.43-7.47 (m, 1H), 6.120-6.16 (m, 1H), 4.61-4.70 (m, 1H), 3.85-3.91 (m, 1H), 2.59-2.71 (m, 7H), 1.26 (s, 6H); MS: m/z=429 (M+H); HPLC retention time: 10.42 min.

### Examples 98a and 98b

### 6,6-dimethyl-N-(1-(1-(thiazol-5-yl)ethyl)-1H-pyrazol-4-yl)-4,5,6,7-tetrahydro-1H-indazole-3-carboxamide

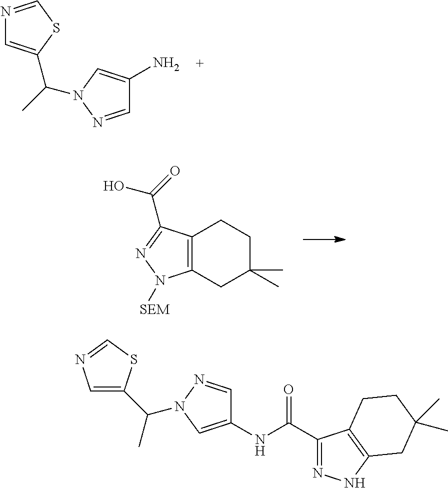 [SMILES: CC(c1cncs1)n1cc(N)cn1 ; CC1(C)CCc2c(C(=O)O)nn(COCC[Si](C)(C)C)c2C1 ; CC(c1cncs1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(thiazol-5-yl)ethyl)-1H-pyrazol-4-amine (Example A40).

SFC conditions: Lux Cellulose-1 (4.6×50 mm, 5 μm particle size) at 30% methanol w/0.1% NH₄OH; 4 mL/min, 120 bars, 40° C.

98a: ¹H NMR (400 MHz, DMSO-d₆) δ 12.79 (s, 1H), 10.09 (s, 1H), 9.00 (d, J=0.7 Hz, 1H), 8.11 (s, 1H), 7.86 (d, J=1.0 Hz, 1H), 7.67 (s, 1H), 5.98 (q, J=6.9 Hz, 1H), 2.66 (t, J=6.4 Hz, 2H), 2.39 (s, 2H), 1.84 (d, J=6.9 Hz, 3H), 1.47 (t, J=6.3 Hz, 2H), 0.96 (s, 6H)); MS: m/z=371 (M+H); SFC retention time: 0.68 min.

98b: ¹H NMR (400 MHz, DMSO-d₆) δ 12.79 (s, 1H), 10.09 (s, 1H), 9.00 (d, J=1.0 Hz, 1H), 8.11 (s, 1H), 7.86 (d, J=0.9 Hz, 1H), 7.67 (s, 1H), 5.98 (d, J=6.9 Hz, 1H), 2.66 (t, J=6.3 Hz, 2H), 2.39 (s, 2H), 1.84 (d, J=6.9 Hz, 3H), 1.47 (t, J=6.4 Hz, 2H), 0.96 (s, 6H)); MS: m/z=371 (M+H); SFC retention time: 0.54 min.

### Examples 99a and 99b

### N-(1-(1-(1-ethyl-1H-pyrazol-4-yl)propyl)-1H-pyrazol-4-yl)-6,6-dimethyl-4,5,6,7-tetrahydro-1H-indazole-3-carboxamide

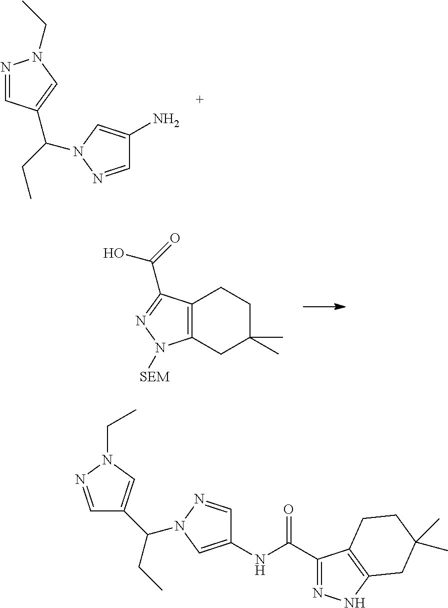

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(1-ethyl-1H-pyrazol-4-yl)propyl)-1H-pyrazol-4-amine (Example A38).

SFC conditions: Chiralpak AD (4.6×50 mm, 5 μm particle size) at 40% methanol w/0.1% NH₄OH; 4 mL/min, 120 bars, 40° C.

99a: ¹H NMR (400 MHz, DMSO-d₆) δ 12.77 (s, 1H), 10.02 (s, 1H), 7.99 (s, 1H), 7.69 (s, 1H), 7.61 (s, 1H), 7.37 (s, 1H), 5.17 (dd, J=9.0, 6.2 Hz, 1H), 4.07 (q, J=7.3 Hz, 2H), 2.66 (t, J=6.3 Hz, 2H), 2.17 (ddd, J=13.7, 9.0, 7.1 Hz, 1H), 2.03 (dt, J=13.8, 6.8 Hz, 1H), 1.47 (t, J=6.4 Hz, 2H), 1.33 (t, J=7.3 Hz, 3H), 0.96 (s, 6H), 0.76 (t, J=7.3 Hz, 3H); MS: m/z=397 (M+H); SFC retention time: 0.79 min.

99b: ¹H NMR (400 MHz, DMSO-d₆) δ 12.77 (s, 1H), 10.02 (s, 1H), 7.99 (s, 1H), 7.69 (s, 1H), 7.61 (s, 1H), 7.37 (d, J=0.6 Hz, 1H), 5.17 (dd, J=9.0, 6.2 Hz, 1H), 4.07 (q, J=7.3 Hz, 2H), 2.66 (t, J=6.4 Hz, 2H), 2.38 (s, 2H), 2.17 (ddd, J=13.8, 9.0, 7.1 Hz, 1H), 2.10-1.94 (m, 1H), 1.47 (t, J=6.4 Hz, 2H), 1.33 (t, J=7.3 Hz, 3H), 0.96 (s, 7H), 0.76 (t, J=7.3 Hz, 3H), 0.96 (s, 6H), 0.76 (t, J=7.3 Hz, 3H); MS: m/z=397 (M+H); SFC retention time: 0.79 min.

### Examples 100a and 100b

### N-(1-(1-(1-ethyl-1H-pyrazol-4-yl)ethyl)-1H-pyrazol-4-yl)-6,6-dimethyl-4,5,6,7-tetrahydro-1H-indazole-3-carboxamide

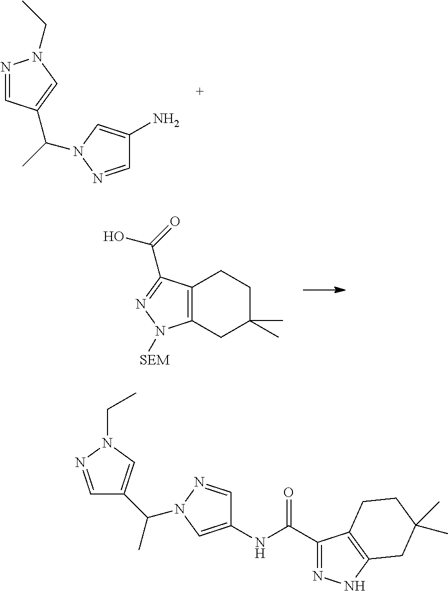

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(1-ethyl-1H-pyrazol-4-yl)ethyl)-1H-pyrazol-4-amine (Example A37).

SFC conditions: Chiralpak AD (4.6×50 mm, 5 μm particle size) at 40% methanol w/0.1% NH₄OH; 4 mL/min, 120 bars, 40° C.

100a: ¹H NMR (400 MHz, DMSO-d₆) δ 12.77 (s, 1H), 10.01 (s, 1H), 7.94 (s, 1H), 7.69 (s, 1H), 7.61 (s, 1H), 7.36 (d, J=0.7 Hz, 1H), 5.46 (q, J=7.0 Hz, 1H), 4.08 (q, J=7.3 Hz, 2H), 2.66 (t, J=6.6 Hz, 2H), 2.38 (s, 2H), 1.70 (d, J=7.0 Hz, 3H), 1.46 (t, J=6.4 Hz, 2H), 1.34 (t, J=7.3 Hz, 3H), 0.96 (s, 7H); MS: m/z=382 (M+H); SFC conditions: 0.61 min.

100b: ¹H NMR (400 MHz, DMSO-d₆) δ 12.77 (s, 1H), 10.01 (s, 1H), 7.94 (s, 1H), 7.69 (s, 1H), 7.61 (s, 1H), 7.36 (d, J=0.7 Hz, 1H), 5.46 (q, J=7.0 Hz, 1H), 4.08 (q, J=7.3 Hz, 2H), 2.66 (t, J=6.6 Hz, 2H), 2.38 (s, 2H), 1.70 (d, J=7.0 Hz, 3H), 1.46 (t, J=6.4 Hz, 2H), 1.34 (t, J=7.3 Hz, 3H), 0.96 (s, 7H); MS: m/z=382 (M+H); SFC conditions: 1.19 min.

### Examples 101a and 101b

### 6,6-dimethyl-N-(1-(1-(5-methyl-1,3,4-oxadiazol-2-yl)ethyl)-1H-pyrazol-4-yl)-4,5,6,7-tetrahydro-1H-indazole-3-carboxamide

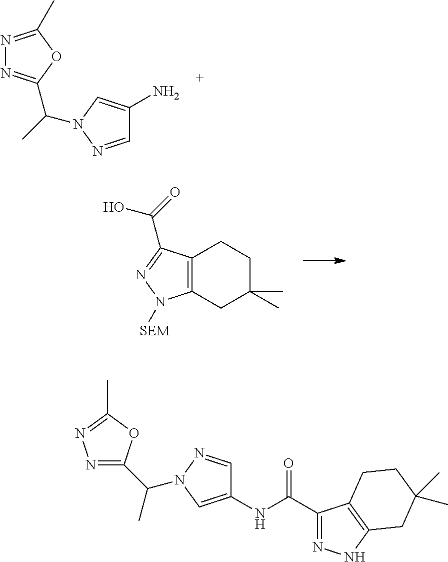 [SMILES: Cc1nnc(C(C)n2cc(N)cn2)o1 ; CC1(C)CCc2c(C(=O)O)nn(COCC[Si](C)(C)C)c2C1 ; Cc1nnc(C(C)n2cc(NC(=O)c3n[nH]c4c3CCC(C)(C)C4)cn2)o1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(5-methyl-1,3,4-oxadiazol-2-yl)ethyl)-1H-pyrazol-4-amine (Example A41).

SFC conditions: Chiralpak AD (4.6×50 mm, 5 μm particle size) at 50% methanol w/0.1% NH₄OH; 4 mL/min, 120 bars, 40° C.

101a: ¹H NMR (400 MHz, DMSO-d₆) δ 12.81 (s, 1H), 10.14 (s, 1H), 8.18 (d, J=0.6 Hz, 1H), 7.69 (s, 1H), 5.94 (q, J=7.0 Hz, 1H), 2.67 (t, J=6.2 Hz, 2H), 2.46 (s, 3H), 2.39 (s, 2H), 1.83 (d, J=7.0 Hz, 3H), 1.48 (t, J=6.4 Hz, 2H), 0.97 (s, 7H); MS: m/z=370 (M+H); SFC retention time: 0.60 min.

101b: ¹H NMR (400 MHz, DMSO-d₆) δ 12.81 (s, 1H), 10.14 (s, 1H), 8.18 (s, 1H), 7.68 (s, 1H), 5.94 (q, J=7.0 Hz, 1H), 2.67 (t, J=6.2 Hz, 2H), 2.46 (s, 3H), 2.39 (s, 2H), 1.83 (d, J=7.0 Hz, 3H), 1.48 (t, J=6.3 Hz, 2H), 0.97 (s, 6H); MS: m/z=370 (M+H); SFC retention time: 0.80 min.

### Examples 102a and 102b

### N-(1-benzyl-1H-pyrazol-4-yl)-6-cyano-6-methyl-4,5,6,7-tetrahydro-1H-indazole-3-carboxamide

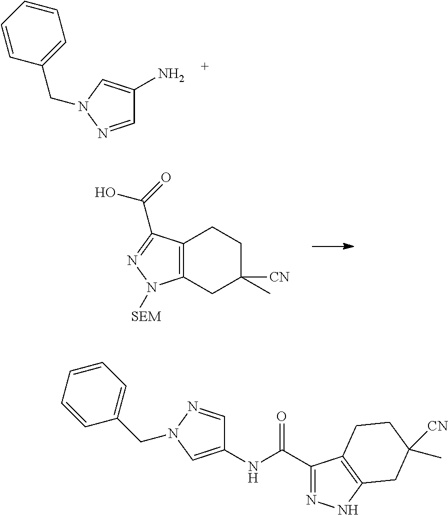 [SMILES: Nc1cnn(Cc2ccccc2)c1 ; CC1(C#N)CCc2c(C(=O)O)nn(COCC[Si](C)(C)C)c2C1 ; CC1(C#N)CCc2c(C(=O)Nc3cnn(Cc4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6-cyano-6-methyl-1-((2-(trimethylsilyl)ethoxy)methyl)-4,5,6,7-tetrahydro-1H-indazole-3-carboxylic acid (Example C36) and 3-((4-amino-1H-pyrazol-1-yl)methyl)benzonitrile (Example A1) with 1-benzyl-1H-pyrazol-4-amine (Example A2). Also, the stereoisomers were separated by preparative chiral HPLC instead of SFC.

Chiral HPLC Conditions: ChiralPak IB-3 (4.6×50 mm, 3 μm particle size); eluent=Hex (0.1% Et₃N):EtOH 90:10; 1.0 ml/min, 4.5 MPA, 25° C.

102a: ¹H NMR (300 HMz, CDCl₃) δ 8.06 (s, 1H), 7.674 (s, 1H), 7.43-7.36 (m, 5H), 5.60 (d, J=10.2, 1H), 3.14-3.08 (m, 1H), 3.0-3.94 (m, 1H), 2.79 (s, 2H), 2.13-2.11 (m, 1H), 1.82-1.80 (m, 1H), 1.51 (s, 3H); MS: m/z=361 (M+H); HPLC retention time: 10.30 min.

102b: ¹H NMR (300 HMz, CDCl₃) δ 8.06 (s, 1H), 7.674 (s, 1H), 7.43-7.36 (m, 5H), 5.60 (d, J=10.2, 1H), 3.14-3.08 (m, 1H), 3.0-3.94 (m, 1H), 2.79 (s, 2H), 2.13-2.11 (m, 1H), 1.82-1.80 (m, 1H), 1.51 (s, 3H); MS: m/z=361 (M+H); HPLC retention time: 12.46 min.

### Examples 103a and 103b

### 6,6-dimethyl-N-(1-(1-(oxazol-2-yl)ethyl)-1H-pyrazol-4-yl)-4,5,6,7-tetrahydro-1H-indazole-3-carboxamide

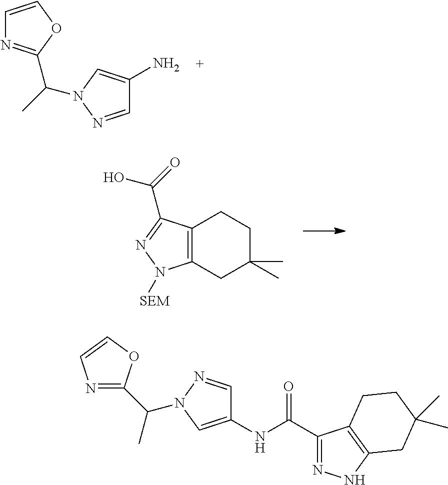 [SMILES: CC(c1ncco1)n1cc(N)cn1 ; CC1(C)CCc2c(C(=O)O)nn(COCC[Si](C)(C)C)c2C1 ; CC(c1ncco1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(oxazol-2-yl)ethyl)-1H-pyrazol-4-amine (Example A42).

SFC conditions: Chiralpak AD (4.6×50 mm, 5 μm particle size) at 45% methanol w/0.1% NH₄OH; 4 mL/min, 120 bars, 40° C.

103a: ¹H NMR (400 MHz, DMSO-d₆) δ 12.78 (s, 1H), 10.10 (s, 1H), 8.21-7.96 (m, 2H), 7.65 (s, 1H), 7.21 (d, J=1.2 Hz, 1H), 5.80 (q, J=7.0 Hz, 1H), 2.67 (t, J=6.4 Hz, 2H), 2.39 (s, 2H), 1.81 (d, J=7.0 Hz, 3H), 1.47 (t, J=6.4 Hz, 2H), 0.97 (s, 7H); MS: m/z=355 (M+H); SFC retention time: 0.76 min.

103b: ¹H NMR (400 MHz, DMSO-d₆) δ 12.78 (s, 1H), 10.10 (s, 1H), 8.24-7.90 (m, 2H), 7.65 (s, 1H), 7.21 (d, J=1.2 Hz, 1H), 5.80 (d, J=7.1 Hz, 1H), 2.67 (t, J=6.4 Hz, 2H), 2.39 (s, 2H), 1.81 (d, J=7.0 Hz, 3H), 1.47 (t, J=6.4 Hz, 2H), 0.97 (s, 7H); MS: m/z=355 (M+H); SFC retention time: 0.97 min.

### Examples 104a and 104b

### 6-cyano-6-methyl-N-(1-(1-(pyridin-2-yl)ethyl)-1H-pyrazol-4-yl)-4,5,6,7-tetrahydro-1H-indazole-3-carboxamide

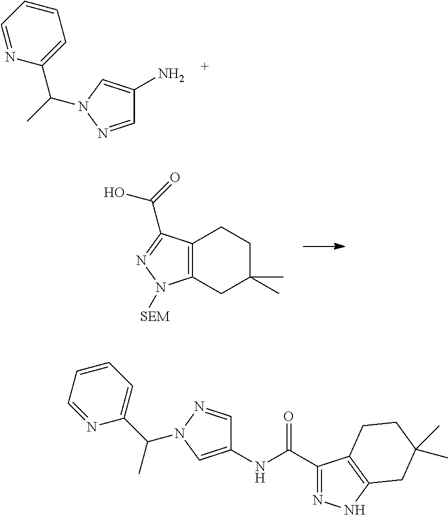 [SMILES: CC(c1ccccn1)n1cc(N)cn1 ; CC1(C)CCc2c(C(=O)O)nn(COCC[Si](C)(C)C)c2C1 ; CC(c1ccccn1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(pyridin-2-yl)ethyl)-1H-pyrazol-4-amine (Example A43).

SFC conditions: Chiralpak IA (4.6×50 mm, 5 μm particle size) at 50% methanol w/0.1% NH₄OH; 4 mL/min, 120 bars, 40° C.

104a: ¹H NMR (400 MHz, DMSO-d₆) δ 12.79 (s, 1H), 10.08 (s, 1H), 8.54 (ddd, J=4.8, 1.9, 0.9 Hz, 1H), 8.12 (s, 1H), 7.74 (td, J=7.7, 1.8 Hz, 1H), 7.67 (s, 1H), 7.29 (ddd, J=7.6, 4.8, 1.2 Hz, 1H), 7.01 (dt, J=7.8, 1.1 Hz, 1H), 5.60 (q, J=7.0 Hz, 1H), 2.67 (t, J=6.4 Hz, 2H), 2.39 (s, 2H), 1.81 (d, J=7.1 Hz, 3H), 1.47 (t, J=6.4 Hz, 2H), 0.96 (s, 7H); MS: m/z=365 (M+H); SFC retention time: 0.37 min.

104b: ¹H NMR (400 MHz, DMSO-d₆) δ 12.79 (s, 1H), 10.08 (s, 1H), 8.54 (ddd, J=4.8, 1.8, 0.9 Hz, 1H), 8.12 (s, 1H), 7.74 (td, J=7.7, 1.8 Hz, 1H), 7.67 (s, 1H), 7.29 (ddd, J=7.5, 4.8, 1.2 Hz, 1H), 7.02 (d, J=7.9 Hz, 1H), 5.60 (q, J=7.1 Hz, 1H), 2.67 (t, J=6.3 Hz, 2H), 2.39 (s, 3H), 1.81 (d, J=7.1 Hz, 3H), 1.47 (t, J=6.4 Hz, 2H), 0.96 (s, 7H); MS: m/z=365 (M+H); SFC retention time: 0.74 min.

### Examples 105a and 105b

### N-(1-benzyl-1H-pyrazol-4-yl)-2-oxo-1′,4′,5′,7′-tetrahydrospiro[cyclopentane-1,6′-indazole]-3′-carboxamide

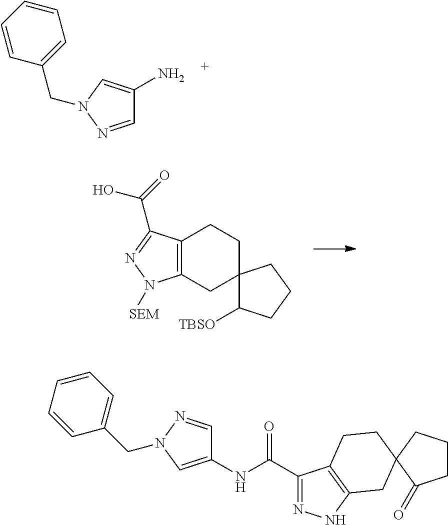 [SMILES: Nc1cnn(Cc2ccccc2)c1 ; CC(C)(C)[Si](C)(C)OC1CCCC12CCc1c(C(=O)O)nn(COCC[Si](C)(C)C)c1C2 ; O=C(Nc1cnn(Cc2ccccc2)c1)c1n[nH]c2c1CCC1(CCCC1=O)C2]

To a solution of 2′-[tert-butyl(dimethyl)silyl]oxy-1-(2-trimethylsilylethoxymethyl)spiro[5,7-dihydro-4H-indazole-6,1′-cyclopentane]-3-carboxylic acid (0.422 g, 0.878 mmol, Example C30), 1-benzylpyrazol-4-amine (1.50 equiv., 1.32 mmol, 228 mg, Example A2) and O-(benzotriazol-1-yl)-N,N,N′,N′-tetramethyluronium tetrafluoroborate (1.40 equiv., 1.23 mmol, 407 mg) in dry N,N-dimethylformamide (3 mL) was added N-ethyldiisopropylamine (3.00 equiv., 2.63 mmol, 0.46 mL). The sample was stirred for 2 hours. The sample was diluted with EtOAc, washed 3 times with H₂O, back extracted 2 times with EtOAc, dried over MgSO₄, filtered, evaporated, and was purified by CombiFlash (12 g, 0-40% EtOAc in heptane, 11 min gradient) to provide N-(1-benzylpyrazol-4-yl)-2′-[tert-butyl(dimethyl)silyl]oxy-1-(2-trimethylsilylethoxymethyl)spiro[5,7-dihydro-4H-indazole-6,1′-cyclopentane]-3-carboxamide (504 mg, 0.79 mmol, 90% yield).

N-(1-benzylpyrazol-4-yl)-2′-[tert-butyl(dimethyl)silyl]oxy-1-(2-trimethylsilylethoxymethyl)spiro[5,7-dihydro-4H-indazole-6,1′-cyclopentane]-3-carboxamide (0.504 g, 0.793 mmol) and trifluoroacetic acid (3 mL) were combined and stirred for 1.5 hours. The sample was then concentrated. The sample was cooled to 0° C. then diluted with ethanol (8 mL) and 5 M NaOH(aq) (8 mL). The sample was allowed to warm slowly to room temperature and stirred overnight. The sample was extracted 9 times with 10% MeOH in dichloromethane, dried over MgSO₄, filtered, and evaporated to provide N-(1-benzylpyrazol-4-yl)-2′-hydroxy-spiro[1,4,5,7-tetrahydroindazole-6,1′-cyclopentane]-3-carboxamide (310 mg, 0.79 mmol, 100% yield).

N-(1-benzylpyrazol-4-yl)-2′-hydroxy-spiro[1,4,5,7-tetrahydroindazole-6,1-cyclopentane]-3-carboxamide (0.2914 g, 0.7443 mmol), pyridinium chlorochromate (2.00 equiv., 1.49 mmol, 327 mg), and dry dichloromethane (4 mL) were combined and stirred for 1 hour. The sample was filtered through Celite, evaporated, then the residue was purified by reverse phase HPLC followed by SFC on a chiral stationary phase to provide the title compounds as single enantiomers.

SFC conditions: Lux Cellulose-3 (4.6×50 mm, 5 μm particle size) at 25% methanol w/0.1% NH₄OH; 4 mL/min, 120 bars, 40° C.

105a: ¹H NMR (400 MHz, DMSO-d₆) δ 12.92 (s, 1H), 10.16 (s, 1H), 8.09 (s, 1H), 7.64 (s, 1H), 7.37-7.26 (m, 3H), 7.26-7.20 (m, 2H), 5.28 (s, 2H), 2.88-2.79 (m, 1H), 2.69-2.21 (m, 5H), 1.93-1.80 (m, 3H), 1.70-1.59 (m, 2H), 1.56-1.45 (m, 1H); MS: m/z=390 (M+H); SFC retention time: 0.60 min.

105b: ¹H NMR (400 MHz, DMSO-d₆) δ 12.92 (s, 1H), 10.16 (s, 1H), 8.09 (s, 1H), 7.64 (d, J=0.5 Hz, 1H), 7.37-7.26 (m, 3H), 7.26-7.20 (m, 2H), 5.28 (s, 2H), 2.89-2.77 (m, 1H), 2.70-2.19 (m, 5H), 1.93-1.80 (m, 3H), 1.70-1.59 (m, 2H), 1.56-1.45 (m, 1H); MS: m/z=390 (M+H);

SFC retention time: 0.97 min.

### Examples 106a and 106b

### 6,6-dimethyl-N-(1-(1-(pyridin-3-yl)ethyl)-1H-pyrazol-4-yl)-4,5,6,7-tetrahydro-1H-indazole-3-carboxamide

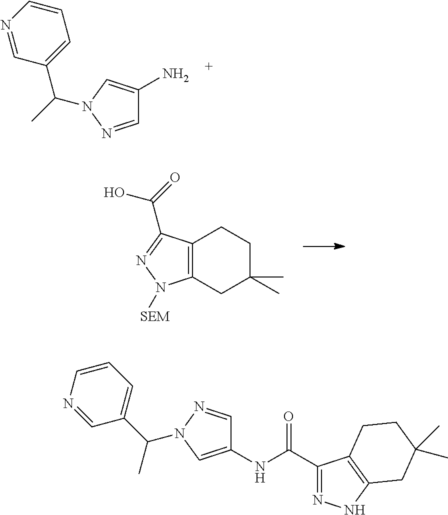 [SMILES: CC(c1cccnc1)n1cc(N)cn1 ; CC1(C)CCc2c(C(=O)O)nn(COCC[Si](C)(C)C)c2C1 ; CC(c1cccnc1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(pyridin-3-yl)ethyl)-1H-pyrazol-4-amine (Example A44).

SFC conditions: Lux Cellulose-1 (4.6×50 mm, 5 μm particle size) at 20% methanol w/0.1% NH₄OH; 4 mL/min, 120 bars, 40° C.

106a: ¹H NMR (400 MHz, DMSO-d₆) δ 12.82 (s, 1H), 10.13 (s, 1H), 8.48 (dd, J=4.8, 1.7 Hz, 2H), 8.15 (d, J=0.6 Hz, 1H), 7.71-7.59 (m, 2H), 7.36 (ddd, J=7.9, 4.8, 0.9 Hz, 1H), 5.65 (q, J=7.0 Hz, 1H), 2.66 (t, J=6.4 Hz, 2H), 2.39 (s, 2H), 1.81 (d, J=7.1 Hz, 3H), 1.47 (t, J=6.4 Hz, 2H), 0.96 (s, 6H); MS: m/z=365 (M+H); SFC retention time: 0.96 min.

106b: ¹H NMR (400 MHz, DMSO-d₆) δ 12.82 (s, 1H), 10.13 (s, 1H), 8.48 (dd, J=4.8, 1.7 Hz, 2H), 8.15 (d, J=0.6 Hz, 1H), 7.80-7.54 (m, 2H), 7.36 (ddd, J=7.9, 4.7, 0.9 Hz, 1H), 5.65 (d, J=7.0 Hz, 1H), 2.66 (t, J=6.5 Hz, 2H), 2.39 (s, 2H), 1.81 (d, J=7.1 Hz, 3H), 1.47 (t, J=6.4 Hz, 2H), 0.96 (s, 6H); MS: m/z=365 (M+H); SFC retention time: 1.19 min.

### Examples 107a and 107b

### 6,6-dimethyl-N-(1-(1-(pyridin-4-yl)ethyl)-1H-pyrazol-4-yl)-4,5,6,7-tetrahydro-1H-indazole-3-carboxamide

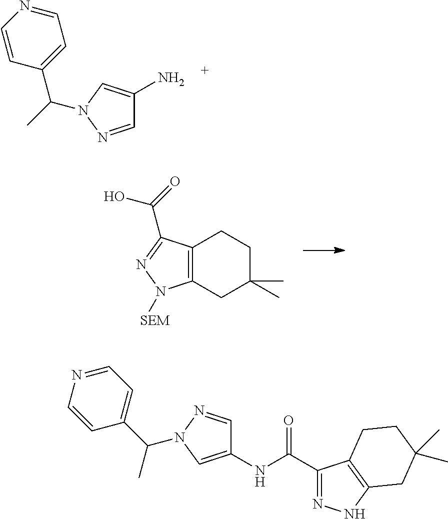 [SMILES: CC(c1ccncc1)n1cc(N)cn1 ; CC1(C)CCc2c(C(=O)O)nn(COCC[Si](C)(C)C)c2C1 ; CC(c1ccncc1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(pyridin-4-yl)ethyl)-1H-pyrazol-4-amine (Example A45).

SFC conditions: Lux Cellulose-3 (4.6×50 mm, 5 μm particle size) at 15% methanol w/0.1% NH₄OH; 4 mL/min, 120 bars, 40° C.

107a: ¹H NMR (400 MHz, DMSO-d₆) δ 12.82 (s, 1H), 10.15 (s, 1H), 8.67-8.34 (m, 2H), 8.16 (d, J=0.6 Hz, 1H), 7.70 (d, J=0.7 Hz, 1H), 7.28-6.96 (m, 2H), 5.63 (d, J=7.1 Hz, 1H), 2.72-2.61 (m, 2H), 2.39 (s, 2H), 1.79 (d, J=7.1 Hz, 3H), 1.47 (t, J=6.4 Hz, 2H), 0.96 (s, 6H); MS: m/z=365 (M+H); SFC retention time: 0.58 min.

107b: ¹H NMR (400 MHz, DMSO-d₆) δ 12.82 (s, 1H), 10.15 (s, 1H), 8.78-8.30 (m, 2H), 8.16 (s, 1H), 7.70 (s, 1H), 7.32-6.92 (m, 2H), 5.63 (d, J=7.1 Hz, 1H), 2.66 (t, J=6.4 Hz, 2H), 2.39 (s, 2H), 1.79 (d, J=7.1 Hz, 3H), 1.47 (t, J=6.4 Hz, 2H), 0.96 (s, 6H); MS: m/z=365 (M+H); SFC retention time: 0.77 min.

### Examples 108a and 108b

### N-(1-(1-(5-fluoropyridin-3-yl)ethyl)-1H-pyrazol-4-yl)-6,6-dimethyl-4,5,6,7-tetrahydro-1H-indazole-3-carboxamide

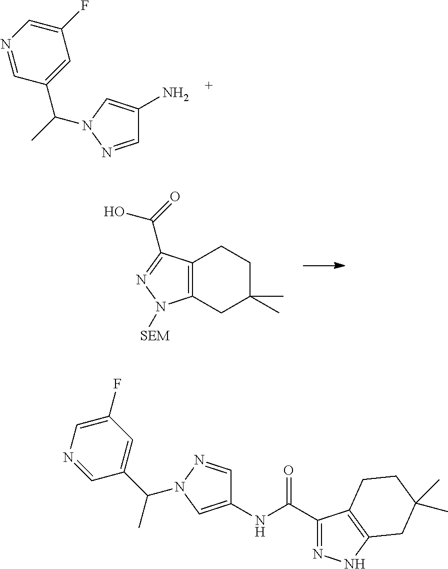 [SMILES: CC(c1cncc(F)c1)n1cc(N)cn1 ; CC1(C)CCc2c(C(=O)O)nn(COCC[Si](C)(C)C)c2C1 ; CC(c1cncc(F)c1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(5-fluoropyridin-3-yl)ethyl)-1H-pyrazol-4-amine (Example A46).

SFC conditions: Chiralpak IA (4.6×50 mm, 5 μm particle size) at 50% methanol w/0.1% NH₄OH; 4 mL/min, 120 bars, 40° C.

108a: ¹H NMR (400 MHz, DMSO-d₆) δ 12.83 (s, 1H), 10.15 (s, 1H), 8.50 (d, J=2.8 Hz, 1H), 8.36 (t, J=1.8 Hz, 1H), 8.20 (d, J=0.7 Hz, 1H), 7.69 (s, 1H), 7.60 (dt, J=10.0, 2.3 Hz, 1H), 5.72 (q, J=7.2 Hz, 1H), 2.73-2.61 (m, 2H), 2.39 (s, 2H), 1.83 (d, J=7.1 Hz, 3H), 1.47 (t, J=6.4 Hz, 2H), 0.96 (s, 6H); MS: m/z=383 (M+H); SFC retention time: 0.84 min.

108b: ¹H NMR (400 MHz, DMSO-d₆) δ 12.83 (s, 1H), 10.15 (s, 1H), 8.50 (d, J=2.8 Hz, 1H), 8.36 (t, J=1.8 Hz, 1H), 8.20 (d, J=0.6 Hz, 1H), 7.69 (s, 1H), 7.60 (dt, J=9.9, 2.4 Hz, 1H), 5.72 (q, J=7.1 Hz, 1H), 2.67 (t, J=6.5 Hz, 2H), 2.39 (s, 2H), 1.83 (d, J=7.1 Hz, 3H), 1.47 (t, J=6.4 Hz, 2H), 0.96 (s, 6H) MS: m/z=383 (M+H); SFC retention time: 1.11 min.

### Examples 109a and 109b

### N-(1-(1-(6-methoxypyridin-3-yl)ethyl)-1H-pyrazol-4-yl)-6,6-dimethyl-4,5,6,7-tetrahydro-1H-indazole-3-carboxamide

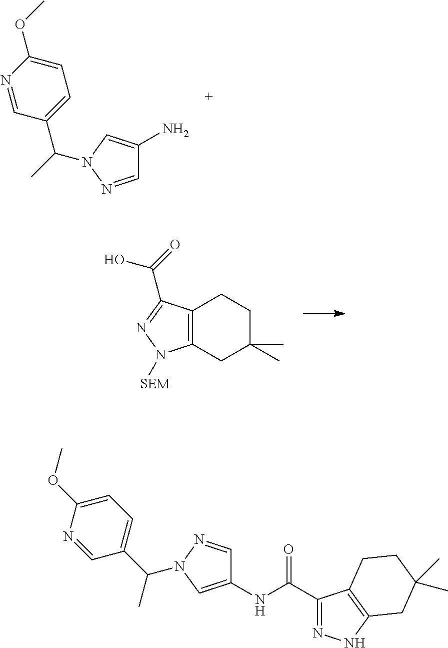 [SMILES: COc1ccc(C(C)n2cc(N)cn2)cn1 ; CC1(C)CCc2c(C(=O)O)nn(COCC[Si](C)(C)C)c2C1 ; COc1ccc(C(C)n2cc(NC(=O)c3n[nH]c4c3CCC(C)(C)C4)cn2)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(1-(6-methoxypyridin-3-yl)ethyl)-1H-pyrazol-4-amine (Example A47).

SFC conditions: Lux Cellulose-3 (4.6×50 mm, 5 μm particle size) at 15% methanol w/0.1% NH₄OH; 4 mL/min, 120 bars, 40° C.

109a: ¹H NMR (400 MHz, DMSO-d₆) δ 12.81 (s, 1H), 10.10 (s, 1H), 8.29-7.95 (m, 2H), 7.86-7.46 (m, 2H), 6.79 (dd, J=8.6, 0.6 Hz, 1H), 5.56 (q, J=7.0 Hz, 1H), 3.82 (s, 3H), 2.66 (t, J=6.3 Hz, 2H), 2.38 (s, 2H), 2.08 (s, 2H), 1.78 (d, J=7.1 Hz, 3H), 1.47 (t, J=6.4 Hz, 2H), 0.96 (s, 6H); MS: m/z=395 (M+H); SFC retention time: 1.06 min.

109b: ¹H NMR (400 MHz, DMSO-d₆) δ 12.81 (s, 1H), 10.10 (s, 1H), 8.31-7.90 (m, 2H), 7.90-7.37 (m, 2H), 6.79 (dd, J=8.5, 0.6 Hz, 1H), 5.56 (q, J=6.9 Hz, 1H), 3.82 (s, 3H), 2.72-2.59 (m, 2H), 2.38 (s, 2H), 1.78 (d, J=7.1 Hz, 3H), 1.46 (t, J=6.3 Hz, 2H), 0.96 (s, 6H); MS: m/z=395 (M+H); SFC retention time: 0.84 min.

### Examples 110a and 110b

### N-(1-((3-cyanophenyl)(1,1-dioxidotetrahydro-2H-thiopyran-4-yl)methyl)-1H-pyrazol-4-yl)-6,6-dimethyl-4,5,6,7-tetrahydro-1H-indazole-3-carboxamide

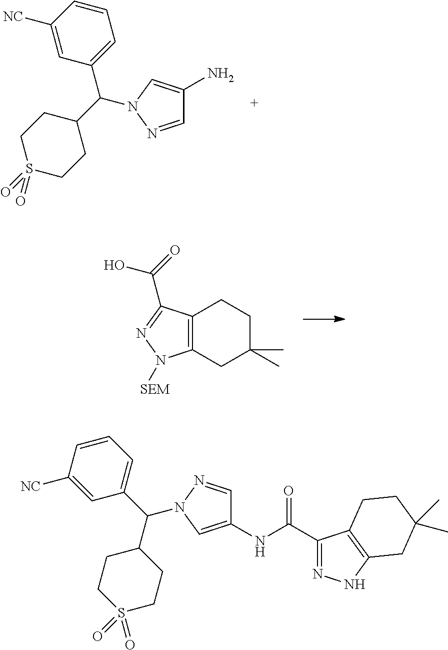 [SMILES: N#Cc1cccc(C(C2CCS(=O)(=O)CC2)n2cc(N)cn2)c1 ; CC1(C)CCc2c(C(=O)O)nn(COCC[Si](C)(C)C)c2C1 ; CC1(C)CCc2c(C(=O)Nc3cnn(C(c4cccc(C#N)c4)C4CCS(=O)(=O)CC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 3-((4-amino-1H-pyrazol-1-yl)(1,1-dioxidotetrahydro-2H-thiopyran-4-yl)methyl)benzonitrile (Example A61). Also, the stereoisomers were separated by preparative chiral HPLC instead of SFC.

Chiral HPLC Conditions: ChiralPak IB-3 (4.6×50 mm, 3 μm particle size); eluent=Hex (0.1% Et₃N):EtOH 60:40; 1.0 ml/min, 4.5 MPA, 25° C.

110a: ¹H NMR (300 HMz, DMSO-d₆) δ 12.81 (s, 1H), 10.15 (s, 1H), 8.20 (s, 1H), 8.02 (s, 1H), 7.89 (d, 1H, J=8.1 Hz), 7.81 (d, 1H, J=7.8 Hz), 7.73 (s, 2H), 7.58-7.63 (m, 1H), 5.44 (d, 1H, J=10.8 Hz), 2.92-3.13 (m, 4H), 2.83-2.87 (m, 1H), 2.64-2.73 (m, 2H), 2.38 (s, 2H), 1.44-1.69 (m, 6H), 1.05 (m, 6H); MS: m/z=507 (M+H).

110b: ¹H NMR (300 HMz, DMSO-d₆) δ 12.82 (s, 1H), 10.15 (s, 1H), 8.21 (s, 1H), 8.02 (s, 1H), 7.89 (d, 1H, J=8.1 Hz), 7.81 (d, 1H, J=7.8 Hz), 7.73 (s, 2H), 7.58-7.63 (m, 1H), 5.44 (d, 1H, J=10.8 Hz), 2.92-3.13 (m, 4H), 2.83-2.87 (m, 1H), 2.64-2.73 (m, 2H), 2.38 (s, 2H), 1.44-1.69 (m, 6H), 1.05 (m, 6H). MS: m/z=507 (M+H).

### Example 111

### N-(1-((2-aminopyridin-4-yl)methyl)-1H-pyrazol-4-yl)-6,6-dimethyl-4,5,6,7-tetrahydro-1H-indazole-3-carboxamide

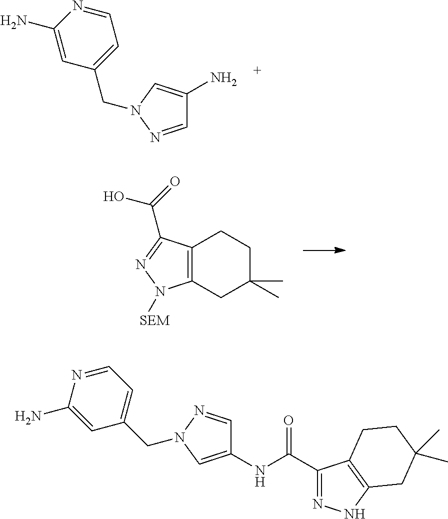 [SMILES: Nc1cnn(Cc2ccnc(N)c2)c1 ; CC1(C)CCc2c(C(=O)O)nn(COCC[Si](C)(C)C)c2C1 ; CC1(C)CCc2c(C(=O)Nc3cnn(Cc4ccnc(N)c4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 4-((4-amino-1H-pyrazol-1-yl)methyl)pyridin-2-amine (Example A62).

111: ¹H NMR (300 MHz, DMSO-d₆) δ 12.83 (s, 1H) 10.18 (s, 1H), 8.10 (s, 1H), 7.82 (d, 1H, J=5.4 Hz), 7.68 (s, 1H), 6.26-6.28 (m, 1H), 6.10 (s, 1H), 5.94 (s, 2H), 5.15 (s, 2H), 2.65-2.69 (m, 2H), 2.39 (s, 2H), 1.45-1.49 (m, 2H), 0.97 (s, 6H). MS: m/z=366 (M+H).

### Example 112

### N-(1-((2-aminopyridin-3-yl)methyl)-1H-pyrazol-4-yl)-6,6-dimethyl-4,5,6,7-tetrahydro-1H-indazole-3-carboxamide

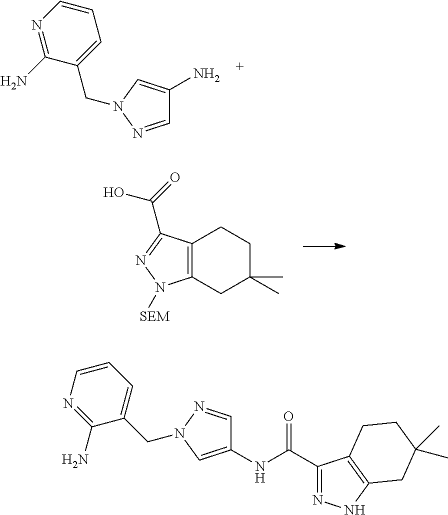 [SMILES: Nc1cnn(Cc2cccnc2N)c1 ; CC1(C)CCc2c(C(=O)O)nn(COCC[Si](C)(C)C)c2C1 ; CC1(C)CCc2c(C(=O)Nc3cnn(Cc4cccnc4N)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 3-((4-amino-1H-pyrazol-1-yl)methyl)pyridin-2-amine (Example A63). 112: ¹H NMR (300 MHz, DMSO-d₆) δ 8.08 (s, 1H), 7.91 (d, J=5.1, 1 H), 7.68 (s, 1H), 7.36 (d, J=6.9, 1 H), 6.72-6.68 (m, 1H), 5.19 (s, 2H), 2.669 (s, 2H), 1.49 (m, 2H), 0.97 (s, 3H). MS: m/z=366 (M+H).

### Examples 113a and 113b

### 6,6-dimethyl-N-(1-(phenyl(tetrahydro-2H-pyran-4-yl)methyl)-1H-pyrazol-4-yl)-4,5,6,7-tetra hydro-1H-indazole-3-carboxamide

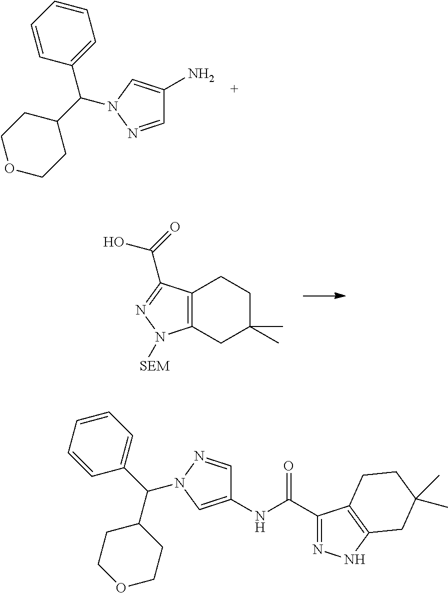 [SMILES: Nc1cnn(C(c2ccccc2)C2CCOCC2)c1 ; CC1(C)CCc2c(C(=O)O)nn(COCC[Si](C)(C)C)c2C1 ; CC1(C)CCc2c(C(=O)Nc3cnn(C(c4ccccc4)C4CCOCC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(phenyl(tetrahydro-2H-pyran-4-yl)methyl)-1H-pyrazol-4-amine (Example A34).

SFC conditions: (S,S) Whelk-O1 (4.6×50 mm, 5 μm particle size) at 40% methanol w/0.1% NH₄OH; 4 mL/min, 120 bars, 40° C.

113a: ¹H NMR (400 MHz, DMSO-d₆) δ 12.77 (s, 1H), 10.03 (s, 1H), 8.14 (s, 1H), 7.64 (s, 1H), 7.57-7.49 (m, 2H), 7.34 (t, J=7.3 Hz, 2H), 7.31-7.24 (m, 1H), 5.04 (d, J=10.7 Hz, 1H), 3.90-3.72 (m, 2H), 2.66 (t, J=6.0 Hz, 3H), 2.38 (s, 2H), 1.47 (t, J=6.4 Hz, 2H), 1.34-1.05 (m, 5H), 0.96 (s, 6H); MS: m/z=434 (M+H); SFC retention time: 1.05 min.

113b: ¹H NMR (400 MHz, DMSO-d₆) δ 12.77 (s, 1H), 10.03 (s, 1H), 8.14 (s, 1H), 7.65 (s, 1H), 7.57-7.48 (m, 2H), 7.38-7.31 (m, 2H), 7.31-7.24 (m, 1H), 5.04 (d, J=10.8 Hz, 1H), 3.90-3.72 (m, 2H), 2.66 (t, J=6.2 Hz, 3H), 2.38 (s, 2H), 1.47 (t, J=6.4 Hz, 2H), 1.33-1.06 (m, 5H), 0.96 (s, 6H); MS: m/z=434 (M+H); MS: m/z=434 (M+H); SFC retention time: 0.46 min.

### Examples 114a and 114b

### 6,6-dimethyl-N-(1-((1-methyl-1H-imidazol-2-yl)(thiophen-2-yl)methyl)-1H-pyrazol-4-yl)-4,5,6,7-tetrahydro-1H-indazole-3-carboxamide

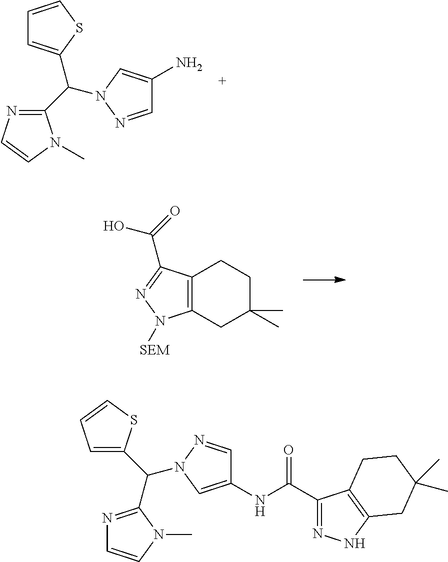 [SMILES: Cn1ccnc1C(c1cccs1)n1cc(N)cn1 ; CC1(C)CCc2c(C(=O)O)nn(COCC[Si](C)(C)C)c2C1 ; Cn1ccnc1C(c1cccs1)n1cc(NC(=O)c2n[nH]c3c2CCC(C)(C)C3)cn1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-((1-methyl-1H-imidazol-2-yl)(thiophen-2-yl)methyl)-1H-pyrazol-4-amine (Example A35).

SFC conditions: Chiralpak IA (4.6×50 mm, 5 μm particle size) at 50% methanol w/0.1% NH₄OH; 4 mL/min, 120 bars, 40° C.

114a: ¹H NMR (400 MHz, DMSO-d₆) δ 12.79 (s, 1H), 10.12 (s, 1H), 8.03 (s, 1H), 7.66 (s, 1H), 7.27 (s, 1H), 7.17 (s, 1H), 7.11 (d, J=3.5 Hz, 1H), 6.99 (dd, J=5.2, 3.3 Hz, 1H), 6.90 (s, 1H), 3.56 (s, 3H), 2.64 (t, J=6.4 Hz, 2H), 2.38 (s, 2H), 1.46 (t, J=6.4 Hz, 2H), 0.95 (s, 6H); MS: m/z=436 (M+H); SFC retention time: 0.46 min.

114b: ¹H NMR (400 MHz, DMSO-d₆) δ 12.79 (s, 1H), 10.12 (s, 1H), 8.03 (s, 1H), 7.66 (s, 1H), 7.53 (d, J=5.1 Hz, 1H), 7.27 (s, 1H), 7.17 (s, 1H), 7.11 (d, J=3.5 Hz, 1H), 6.99 (dd, J=5.3, 3.4 Hz, 1H), 6.90 (s, 1H), 3.56 (s, 4H), 2.64 (t, J=6.4 Hz, 2H), 2.38 (s, 2H), 1.46 (t, J=6.4 Hz, 2H), 0.95 (s, 6H); MS: m/z=436 (M+H); SFC retention time: 0.65 min.

### Examples 115a and 115b

### N-(1-((3-chlorophenyl)(tetrahydro-2H-pyran-4-yl)methyl)-1H-pyrazol-4-yl)-6,6-dimethyl-4,5,6,7-tetrahydro-1H-indazole-3-carboxamide

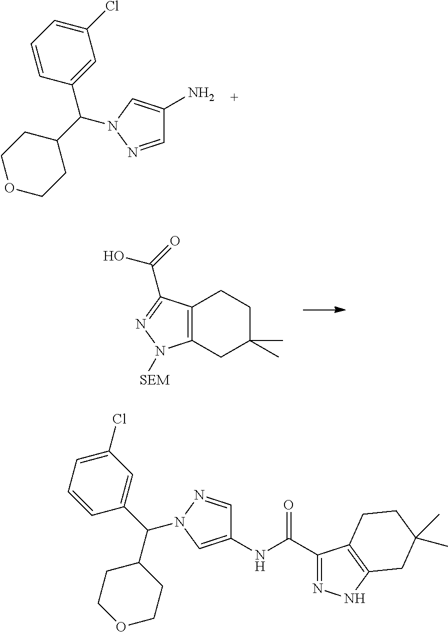 [SMILES: Nc1cnn(C(c2cccc(Cl)c2)C2CCOCC2)c1 ; CC1(C)CCc2c(C(=O)O)nn(COCC[Si](C)(C)C)c2C1 ; CC1(C)CCc2c(C(=O)Nc3cnn(C(c4cccc(Cl)c4)C4CCOCC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-((3-chlorophenyl)(tetrahydro-2H-pyran-4-yl)methyl)-1H-pyrazol-4-amine (Example A48).

SFC conditions: Lux Cellulose-1 (4.6×50 mm, 5 μm particle size) at 30% methanol w/0.1% NH₄OH; 4 mL/min, 120 bars, 40° C.

115a: ¹H NMR (400 MHz, DMSO-d₆) δ 12.78 (s, 1H), 10.06 (s, 1H), 8.16 (s, 1H), 7.68 (s, 1H), 7.61 (t, J=1.8 Hz, 1H), 7.50 (dt, J=7.3, 1.6 Hz, 1H), 7.42-7.31 (m, 2H), 5.11 (d, J=10.6 Hz, 1H), 3.80 (t, J=10.9 Hz, 2H), 3.28 (s, 3H), 2.74-2.59 (m, 3H), 2.38 (s, 2H), 1.47 (t, J=6.3 Hz, 2H), 1.34-1.02 (m, 4H), 0.96 (s, 6H); MS: m/z=469 (M+H); SFC retention time: 0.66 min.

115b: ¹H NMR (400 MHz, DMSO-d₆) δ 12.78 (s, 1H), 10.06 (s, 1H), 8.16 (s, 1H), 7.68 (s, 1H), 7.61 (t, J=1.8 Hz, 1H), 7.50 (dt, J=7.3, 1.6 Hz, 1H), 7.42-7.31 (m, 2H), 5.11 (d, J=10.6 Hz, 1H), 3.80 (t, J=10.9 Hz, 2H), 3.28 (s, 3H), 2.74-2.59 (m, 3H), 2.38 (s, 2H), 1.47 (t, J=6.3 Hz, 2H), 1.34-1.02 (m, 4H), 0.96 (s, 6H); MS: m/z=469 (M+H); SFC retention time: 0.79 min.

### Examples 116a and 116b

### N-(1-((1,1-dioxidothietan-3-yl)(pyridin-3-yl)methyl)-1H-pyrazol-4-yl)-5a-methyl-1,4,4a,5,5a,6-hexahydrocyclopropa[f]indazole-3-carboxamide

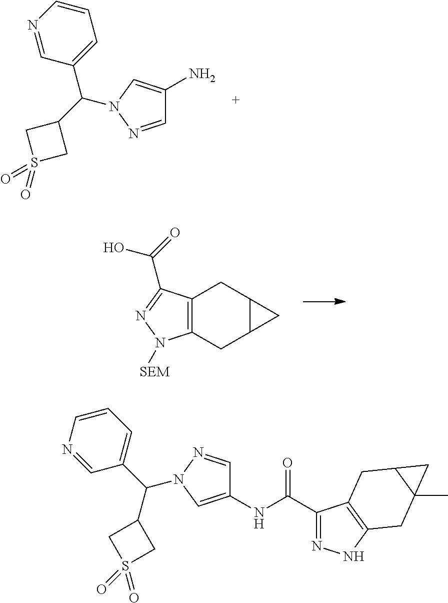 [SMILES: Nc1cnn(C(c2cccnc2)C2CS(=O)(=O)C2)c1 ; C[Si](C)(C)CCOCn1nc(C(=O)O)c2c1CC1CC1C2 ; CC12Cc3[nH]nc(C(=O)Nc4cnn(C(c5cccnc5)C5CS(=O)(=O)C5)c4)c3CC1C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5a-methyl-1-((2-(trimethylsilyl)ethoxy)methyl)-1,4,4a,5,5a,6-hexahydrocyclopropa[f]indazole-3-carboxylic acid (Example C31a) and 3-((4-amino-1H-pyrazol-1-yl)methyl)benzonitrile (Example A1) with 3-((4-amino-1H-pyrazol-1-yl)(pyridin-3-yl)methyl)thietane 1,1-dioxide (Example A64). Also, the stereoisomers were separated by preparative chiral HPLC instead of SFC.

Chiral HPLC Conditions: ChiralPak IC-3 (4.6×50 mm, 3 μm particle size); eluent=DCM:EtOH 95:5; 1.0 ml/min, 5.0 MPA, 25° C.

116a: ¹H NMR (300 MHz, DMSO-d₆) δ 12.92 (s, 1H), 10.17 (s, 1H), 8.70 (s, 1H), 8.52-8.54 (m, 1H), 8.23 (s, 1H), 7.91 (d, 1H, J=7.8 Hz), 7.69 (s, 1H), 7.39-7.42 (m, 1H), 5.77 (d, 1H, J=10.5 Hz), 4.09-4.19 (m, 2H), 3.72-3.94 (m, 3H), 3.15-3.25 (m, 1H), 2.70-2.93 (m, 2H), 2.49-2.51 (m, 1H), 1.21 (s, 3H), 0.99-1.04 (m, 1H), 0.31-0.36 (m, 1H), 0.07-0.09 (m, 1H); MS: m/z=453 (M+H); HPLC retention time: 2.84 min.

116b: ¹H NMR (300 MHz, DMSO-d₆) δ 12.92 (s, 1H), 10.17 (s, 1H), 8.70 (s, 1H), 8.53 (d, 1H, J=4.8 Hz), 8.23 (s, 1H), 7.91 (d, 1H, J=7.8 Hz), 7.69 (s, 1H), 7.35-7.45 (m, 1H), 5.77 (d, 1H, J=10.5 Hz), 4.09-4.22 (m, 2H), 3.72-3.94 (m, 3H), 3.16-3.26 (m, 1H), 2.70-2.98 (m, 2H), 2.50-2.51 (m, 1H), 1.21 (s, 3H), 1.01-1.04 (m, 1H), 0.32-0.36 (m, 1H), 0.07-0.09 (m, 1H); MS: m/z=453 (M+H); HPLC retention time: 4.42 min.

### Examples 117a and 117b

### N-(1-((1,1-dioxidothietan-3-yl)(pyridin-3-yl)methyl)-1H-pyrazol-4-yl)-6,6-dimethyl-4,5,6,7-tetrahydro-1H-indazole-3-carboxamide

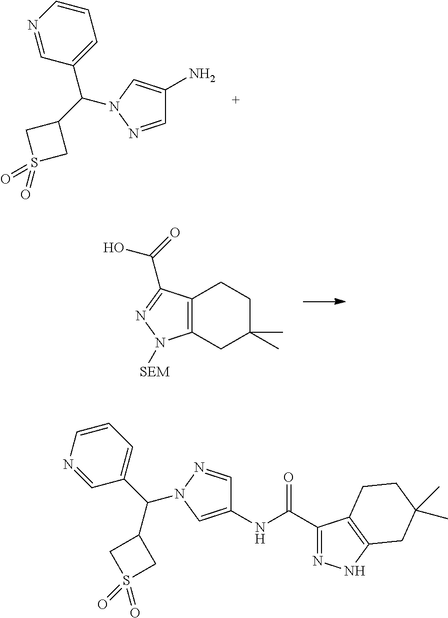 [SMILES: Nc1cnn(C(c2cccnc2)C2CS(=O)(=O)C2)c1 ; CC1(C)CCc2c(C(=O)O)nn(COCC[Si](C)(C)C)c2C1 ; CC1(C)CCc2c(C(=O)Nc3cnn(C(c4cccnc4)C4CS(=O)(=O)C4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 3-((4-amino-1H-pyrazol-1-yl)(pyridin-3-yl)methyl)thietane 1,1-dioxide (Example A64). Also, the stereoisomers were separated by preparative chiral HPLC instead of SFC.

Chiral HPLC Conditions: ChiralPak IB-3 (4.6×50 mm, 3 μm particle size); eluent=Hex:EtOH 60:40; 1.0 ml/min, 6.5 MPA, 25° C.

117a: ¹H NMR (300 MHz, DMSO-d₆) δ 12.83 (s, 1H), 10.17 (s, 1H), 8.71 (s, 1H), 8.53 (d, 1H, J=3.6 Hz), 8.24 (s, 1H), 7.92 (d, 1H, J=8.1 Hz), 7.70 (s, 1H), 7.35-7.45 (m, 1H), 5.77 (d, 1H, J=10.5 Hz), 4.09-4.22 (m, 2H), 3.72-3.94 (m, 3H), 2.54-2.66 (m, 1H), 2.38-2.50 (m, 2H), 1.44-1.48 (m, 2H), 0.96 (s, 6H); MS: m/z=455 (M+H); HPLC retention time: 2.80 min.

117b: ¹H NMR (300 MHz, DMSO-d₆) δ 12.83 (s, 1H), 10.18 (s, 1H), 8.71 (s, 1H), 8.52-8.54 (m, 1H), 8.24 (s, 1H), 7.92 (d, 1H, J=8.1 Hz), 7.70 (s, 1H), 7.40-7.42 (m, 1H), 5.77 (d, 1H, J=10.8 Hz), 4.12-4.16 (m, 2H), 3.72-3.94 (m, 3H), 2.63-2.67 (m, 1H), 2.38-2.50 (m, 2H), 1.44-1.48 (m, 2H), 0.96 (s, 6H); MS: m/z=455 (M+H); HPLC retention time: 4.64 min.

### Example 118

### N-(1-benzyl-1H-pyrazol-4-yl)-1,4,5,7-tetrahydrospiro[indazole-6,3′-thietane]-3-carboxamide

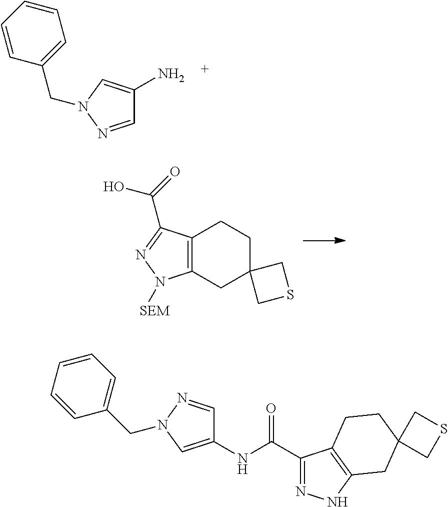 [SMILES: Nc1cnn(Cc2ccccc2)c1 ; C[Si](C)(C)CCOCn1nc(C(=O)O)c2c1CC1(CC2)CSC1 ; O=C(Nc1cnn(Cc2ccccc2)c1)c1n[nH]c2c1CCC1(CSC1)C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1,4,5,7-tetrahydrospiro[indazole-6,3′-thietane]-3-carboxylic acid (Example C29) and 3-((4-amino-1H-pyrazol-1-yl)methyl)benzonitrile (Example A1) with 1-benzyl-1H-pyrazol-4-amine (Example A2).

118: ¹H NMR (400 MHz, DMSO-d₆) δ 12.90 (s, 1H), 10.09 (s, 1H), 8.06 (s, 1H), 7.64 (s, 1H), 7.36-7.26 (m, 3H), 7.25-7.20 (m, 2H), 5.27 (s, 2H), 3.04 (d, J=9.2 Hz, 2H), 2.92 (s, 2H), 2.88 (d, J=9.2 Hz, 2H), 2.72 (t, J=6.3 Hz, 2H), 1.93 (t, J=6.4 Hz, 2H). MS: m/z=380 (M+H).

### Example 119a and 119b

### N-(1-((1,1-dioxidotetrahydro-2H-thiopyran-4-yl)(3-(2-hydroxypropan-2-yl)phenyl)methyl)-1H-pyrazol-4-yl)-6,6-dimethyl-4,5,6,7-tetrahydro-1H-indaznole-3-carboxamide

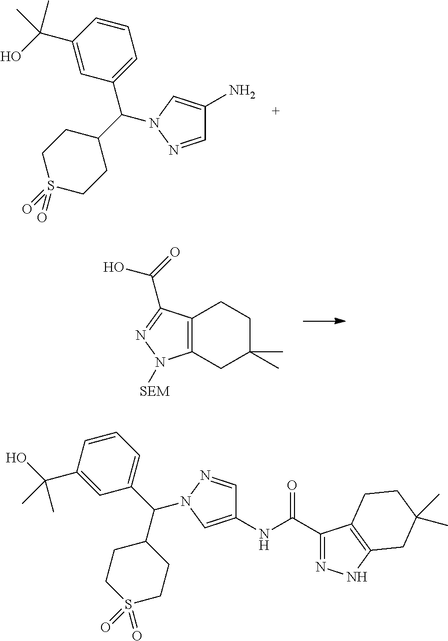 [SMILES: CC(C)(O)c1cccc(C(C2CCS(=O)(=O)CC2)n2cc(N)cn2)c1 ; CC1(C)CCc2c(C(=O)O)nn(COCC[Si](C)(C)C)c2C1 ; CC1(C)CCc2c(C(=O)Nc3cnn(C(c4cccc(C(C)(C)O)c4)C4CCS(=O)(=O)CC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 4-((4-amino-1H-pyrazol-1-yl)(3-(2-hydroxypropan-2-yl)phenyl)methyl)tetrahydro-2H-thiopyran 1,1-dioxide (Example A65). Also, the stereoisomers were separated by preparative chiral HPLC instead of SFC.

Chiral HPLC Conditions: ChiralPak IC-3 (4.6×50 mm, 3 μm particle size); eluent=Hex:EtOH 60:40; 1.0 ml/min, 8.3 MPA, 25° C.

119a: ¹H NMR (300 HMz, CD₃OD) δ 8.14 (s, 1H), 7.68-7.78 (m, 2H), 7.41-7.46 (m, 2H), 7.31-7.36 (m, 1H), 5.12-5.15 (d, 1H, J=10.8), 3.01-3.26 (m, 4H), 2.76-2.87 (m, 2H), 2.43 (s, 2H), 1.81-1.99 (m, 4H), 1.57-1.59 (m, 8H), 1.05 (s, 6H); MS: m/z=540 (M+H); HPLC retention time: 2.95 min.

119b: ¹H NMR (300 HMz, CD₃OD) δ 8.14 (s, 1H), 7.68-7.69 (m, 2H), 7.43-7.50 (m, 2H), 7.30-7.40 (m, 1H), 5.12-5.15 (d, 1H, J=10.8), 3.01-3.16 (m, 3H), 2.86-2.96 (m, 1H), 2.78-2.80 (m, 2H), 2.43 (s, 2H), 1.80-1.82 (m, 4H), 1.53-1.57 (m, 8H), 1.03 (s, 6H); MS: m/z=540 (M+H); HPLC retention time: 4.64 min.

### Examples 120a and 120b

### 5a-methyl-N-(1-((1-oxidotetrahydro-2H-thiopyran-4-yl)(phenyl)methyl)-1H-pyrazol-4-yl)-1,4,4a,5,5a,6-hexahydrocyclopropa[f]indazole-3-carboxamide

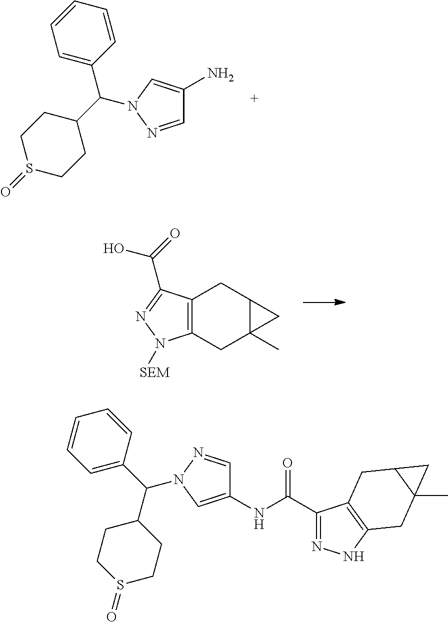 [SMILES: Nc1cnn(C(c2ccccc2)C2CCS(=O)CC2)c1 ; CC12Cc3c(c(C(=O)O)nn3COCC[Si](C)(C)C)CC1C2 ; CC12Cc3[nH]nc(C(=O)Nc4cnn(C(c5ccccc5)C5CCS(=O)CC5)c4)c3CC1C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5a-methyl-1-((2-(trimethylsilyl)ethoxy)methyl)-1,4,4a,5,5a,6-hexahydrocyclopropa[f]indazole-3-carboxylic acid (Example C31a) and 3-((4-amino-1H-pyrazol-1-yl)methyl)benzonitrile (Example A1) with 4-((4-amino-1H-pyrazol-1-yl)(phenyl)methyl)tetrahydro-2H-thiopyran 1-oxide (Example A66). Only the enantiomer of A66 which provided Example 86a was utilized thus resulted in only 2 diastereomers. Also, the stereoisomers were separated by preparative chiral HPLC instead of SFC.

Chiral HPLC Conditions: ChiralPak IC-3 (4.6×50 mm, 3 μm particle size); eluent=Hex:EtOH 50:50; 1.0 ml/min, 4.2 MPA, 25° C.

120a: ¹H NMR (300 MHz, CD₃OD) δ 8.15 (s, 1H), 7.70-7.85 (m, 1H), 7.53-7.69 (m, 2H), 7.29-7.62 (m, 3H), 5.03-5.06 (d, 1H, J=11.1), 3.25 (s, 1H), 2.94-3.08 (m, 4H), 2.66-2.86 (m, 4H), 1.89-2.23 (m, 2H), 1.42-1.47 (m, 2H), 1.22 (s, 3H), 1.06-1.12 (m, 1H), 0.37-0.41 (m, 1H), 0.17-0.20 (m, 1H); MS: m/z=464 (M+H); HPLC retention time: 2.61 min.

120b: ¹H NMR (300 MHz, DMSO-d₆) δ 12.86 (s, 1H), 10.09 (s, 1H), 8.15 (s, 1H), 7.65 (s, 1H), 7.53-7.55 (m, 2H), 7.26-7.38 (m, 3H), 5.27-5.31 (d, 1H, J=10.8), 3.12-3.23 (m, 3H), 2.81-2.99 (m, 2H), 2.51-2.70 (m, 4H), 1.70-1.75 (m, 2H), 1.21-1.32 (m, 5H), 1.01-1.05 (m, 1H), 0.31-0.41 (m, 1H), 0.11-0.15 (m, 1H); MS: m/z=464 (M+H); HPLC retention time: 9.36 min.

### Examples 121a and 121b

### N-(1-(3-(3,3-difluoroazetidin-1-yl)-1-phenylpropyl)-1H-pyrazol-4-yl)-6,6-dimethyl-4,5,6,7-tetrahydro-1H-indazole-3-carboxamide

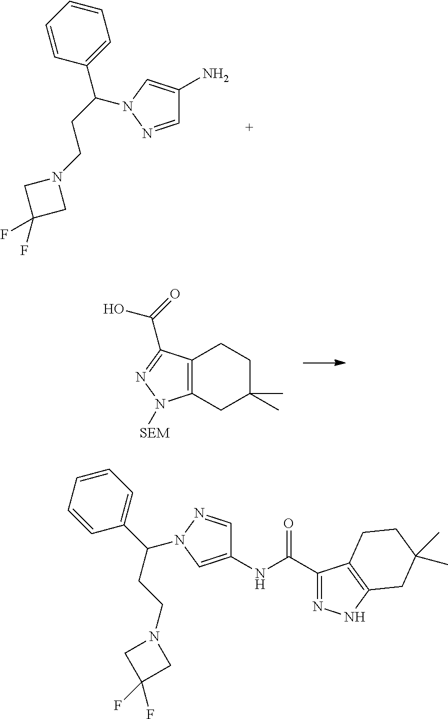 [SMILES: Nc1cnn(C(CCN2CC(F)(F)C2)c2ccccc2)c1 ; CC1(C)CCc2c(C(=O)O)nn(COCC[Si](C)(C)C)c2C1 ; CC1(C)CCc2c(C(=O)Nc3cnn(C(CCN4CC(F)(F)C4)c4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(3-(3,3-difluoroazetidin-1-yl)-1-phenylpropyl)-1H-pyrazol-4-amine (Example A27). Also, instead of performing chiral separation at the final stage, the nitropyrazoles (A27) were resolved by SFC (SFC conditions: Chiralpak AD (4.6×50 mm, 5 μm particle size) at 30% methanol w/0.1% NH₄OH; 4 mL/min, 120 bars, 40° C.) and the respective enantiomers were carried forward to the title compounds. The faster eluting enantiomer of A27 led to 121b, and the slower eluting enantiomer led to 121a.

121a: ¹H NMR (400 MHz, DMSO-d₆) δ 12.99-12.57 (s, 1H), 10.26-9.85 (s, 1H), 8.24-7.94 (s, 1H), 7.81-7.52 (s, 1H), 7.38-7.30 (d, J=4.3 Hz, 4H), 7.30-7.23 (m, 1H), 5.53-5.34 (dd, J=8.3, 5.6 Hz, 1H), 3.65-3.43 (td, J=12.5, 2.9 Hz, 4H), 2.74-2.60 (t, J=6.1 Hz, 2H), 2.47-2.28 (m, 5H), 2.19-1.93 (m, 1H), 1.52-1.39 (t, J=6.4 Hz, 2H), 1.01-0.90 (s, 6H); MS: m/z=469 (M+H).

121b: ¹H NMR (400 MHz, DMSO-d₆) δ 12.85-12.63 (s, 1H), 10.19-9.91 (s, 1H), 8.20-7.97 (s, 1H), 7.79-7.52 (s, 1H), 7.37-7.31 (d, J=4.3 Hz, 4H), 7.30-7.23 (m, 1H), 5.50-5.35 (dd, J=8.2, 5.7 Hz, 1H), 3.65-3.43 (td, J=12.5, 3.0 Hz, 4H), 2.76-2.58 (t, J=6.1 Hz, 2H), 2.47-2.31 (m, 5H), 2.18-1.99 (m, 1H), 1.55-1.33 (t, J=6.4 Hz, 2H), 1.03-0.85 (s, 6H); MS: m/z=469 (M+H).

### Example 122

### 6,6-dimethyl-N-(1-(3-(N-methylmethylsulfonamido)-1-phenylpropyl)-1H-pyrazol-4-yl)-4,5,6,7-tetrahydro-1H-indazole-3-carboxamide

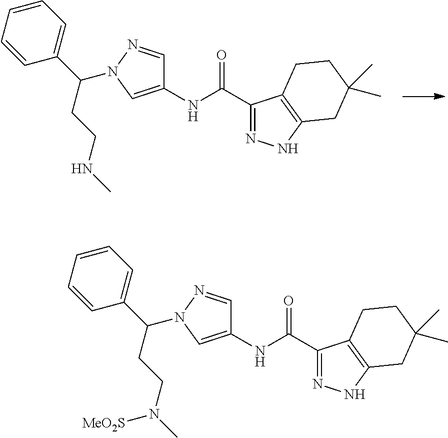 [SMILES: CNCCC(c1ccccc1)n1cc(NC(=O)c2n[nH]c3c2CCC(C)(C)C3)cn1 ; CN(CCC(c1ccccc1)n1cc(NC(=O)c2n[nH]c3c2CCC(C)(C)C3)cn1)S(C)(=O)=O]

To a solution of 6,6-dimethyl-N-(1-(3-(methylamino)-1-phenylpropyl)-1H-pyrazol-4-yl)-4,5,6,7-tetrahydro-1H-indazole-3-carboxamide (Example 39a) in THF was added triethylamine (2.0 equiv.) and methanesulfonyl chloride (1.0 equiv.). The mixture was stirred for 60 minutes, then concentrated in vacuo and purified by reverse phase HPLC to provide 6,6-dimethyl-N-(1-(3-(N-methylmethylsulfonamido)-1-phenylpropyl)-1H-pyrazol-4-yl)-4,5,6,7-tetrahydro-1H-indazole-3-carboxamide (Example 122).

122: ¹H NMR (400 MHz, DMSO-d₆) δ 12.89-12.58 (s, 1H), 10.21-9.85 (s, 1H), 8.12-8.08 (s, 1H), 7.71-7.67 (s, 1H), 7.39-7.22 (m, 5H), 5.47-5.37 (dd, J=9.6, 5.4 Hz, 1H), 3.12-3.00 (m, 1H), 3.00-2.86 (m, 1H), 2.85-2.81 (s, 3H), 2.77-2.74 (s, 3H), 2.72-2.59 (m, 3H), 2.41-2.22 (m, 3H), 1.51-1.42 (t, J=6.3 Hz, 2H), 0.98-0.93 (s, 6H). MS: m/z=485 (M+H).

### Examples 123a and 123b

### N-(1-benzyl-1H-pyrazol-4-yl)-1′-methyl-2′-oxo-1,4,5,7-tetrahydrospiro[indazole-6,3′-pyrrolidine]-3-carboxamide

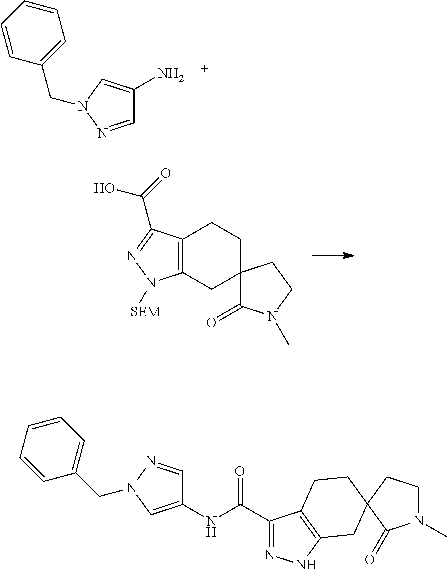 [SMILES: Nc1cnn(Cc2ccccc2)c1 ; CN1CCC2(CCc3c(C(=O)O)nn(COCC[Si](C)(C)C)c3C2)C1=O ; CN1CCC2(CCc3c(C(=O)Nc4cnn(Cc5ccccc5)c4)n[nH]c3C2)C1=O]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1′-methyl-2′-oxo-1-((2-(trimethylsilyl)ethoxy)methyl)-1,4,5,7-tetrahydrospiro[indazole-6,3′-pyrrolidine]-3-carboxylic acid (Example C25) and 3-((4-amino-1H-pyrazol-1-yl)methyl)benzonitrile (Example A1) with 1-benzyl-1H-pyrazol-4-amine (Example A2).

SFC conditions: Lux Cellulose-3 (4.6×50 mm, 5 μm particle size) at 32% methanol w/0.1% NH₄OH; 5 mL/min, 120 bars, 40° C.

123a: ¹H NMR (400 MHz, DMSO-d₆) δ 12.90 (s, 1H), 10.11 (s, 1H), 8.10-8.06 (m, 1H), 7.67-7.63 (m, 1H), 7.37-7.20 (m, 5H), 5.28 (s, 2H), 3.30 (d, J=5.7 Hz, 2H), 2.95-2.82 (m, 1H), 2.77 (s, 3H), 2.72 (d, J=16.2 Hz, 1H), 2.68-2.52 (m, 2H), 1.93-1.84 (m, 1H), 1.73-1.54 (m, 3H); MS: m/z=405.2 (M+H); SFC retention time: 0.42 min.

123b: ¹H NMR (400 MHz, DMSO-d₆) δ 12.91 (s, 1H), 10.11 (s, 1H), 8.08-8.07 (m, 1H), 7.65-7.64 (m, 1H), 7.37-7.20 (m, 5H), 5.28 (s, 2H), 3.36-3.24 (m, 2H), 2.94-2.85 (m, 1H), 2.77 (s, 3H), 2.72 (d, J=16.0 Hz, 1H), 2.68-2.52 (m, 2H), 1.94-1.83 (m, 1H), 1.73-1.54 (m, 3H); MS: m/z=405.2 (M+H); SFC retention time: 0.66 min.

### Example 124

### N-(1-benzyl-1H-pyrazol-4-yl)-1,4,5,6,7,8-hexahydrocyclohepta[c]pyrazole-3-carboxamide

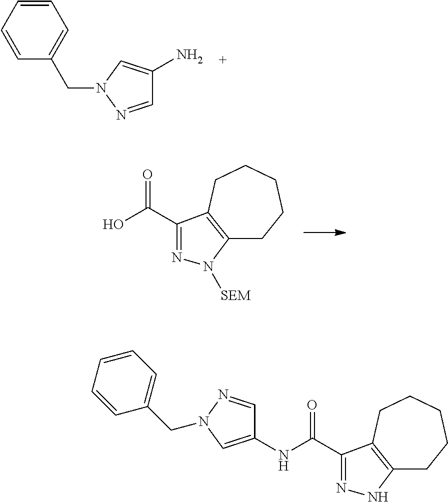 [SMILES: Nc1cnn(Cc2ccccc2)c1 ; C[Si](C)(C)CCOCn1nc(C(=O)O)c2c1CCCCC2 ; O=C(Nc1cnn(Cc2ccccc2)c1)c1n[nH]c2c1CCCCC2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 1-((2-(trimethylsilyl)ethoxy)methyl)-1,4,5,6,7,8-hexahydrocyclohepta[c]pyrazole-3-carboxylic acid (Example C42) and 3-((4-amino-1H-pyrazol-1-yl)methyl)benzonitrile (Example A1) with 1-benzyl-1H-pyrazol-4-amine (Example A2).

124: ¹H NMR (400 MHz, DMSO-d₆) δ 10.21-9.76 (s, 1H), 8.07-8.03 (s, 1H), 7.66-7.61 (s, 1H), 7.38-7.17 (m, 5H), 5.30-5.23 (s, 2H), 2.97-2.87 (m, 2H), 2.75-2.68 (m, 2H), 1.85-1.74 (m, 2H), 1.66-1.47 (m, 4H). MS: m/z=336 (M+H).

### Examples 125a and 125b

### N-(1-(cyclopropyl(3-(methylsulfonyl)phenyl)methyl)-1H-pyrazol-4-yl)-6,6-dimethyl-4,5,6,7-tetrahydro-1H-indazole-3-carboxamide

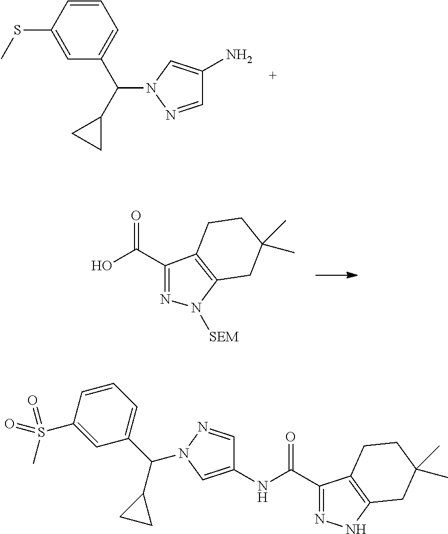 [SMILES: CSc1cccc(C(C2CC2)n2cc(N)cn2)c1 ; CC1(C)CCc2c(C(=O)O)nn(COCC[Si](C)(C)C)c2C1 ; CC1(C)CCc2c(C(=O)Nc3cnn(C(c4cccc(S(C)(=O)=O)c4)C4CC4)c3)n[nH]c2C1]

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with 1-(cyclopropyl(3-(methylthio)phenyl)methyl)-1H-pyrazol-4-amine (Example A67).

SFC conditions: (S,S) Whelk-O1 (4.6×50 mm, 5 μm particle size) at 40% methanol w/0.1% NH₄OH; 5 mL/min, 120 bars, 40° C.

124a: ¹H NMR (400 MHz, DMSO-d₆) δ 12.89-12.70 (s, 1H), 10.19-9.99 (s, 1H), 8.29-8.25 (s, 1H), 7.89-7.81 (m, 2H), 7.70-7.59 (m, 3H), 4.87-4.80 (d, J=10.0 Hz, 1H), 3.23-3.19 (s, 3H), 2.72-2.63 (t, J=6.3 Hz, 2H), 2.42-2.36 (s, 2H), 1.85-1.69 (m, 1H), 1.53-1.42 (t, J=6.3 Hz, 2H), 1.01-0.92 (s, 6H), 0.74-0.61 (m, 2H), 0.57-0.39 (m, 2H); MS: m/z=468 (M+H); SFC retention time: 0.77 min.

124b: ¹H NMR (400 MHz, DMSO-d₆) δ 12.89-12.72 (s, 1H), 10.18-9.99 (s, 1H), 8.36-8.19 (s, 1H), 7.95-7.75 (m, 2H), 7.71-7.51 (m, 3H), 4.91-4.71 (d, J=9.9 Hz, 1H), 3.22-3.18 (s, 3H), 2.71-2.62 (t, J=6.2 Hz, 2H), 2.41-2.37 (s, 2H), 1.85-1.71 (m, 1H), 1.50-1.43 (t, J=6.3 Hz, 2H), 1.00-0.94 (s, 6H), 0.73-0.63 (m, 2H), 0.57-0.38 (m, 2H); MS: m/z=468 (M+H); SFC retention time: 1.34 min.

### Examples 126a-d

### 6,6-dimethyl-N-(1-((3-(methylsulfonyl)phenyl)(tetrahydrofuran-3-yl)methyl)-1H-pyrazol-4-yl)-4,5,6,7-tetrahydro-1H-indazole-3-carboxamide

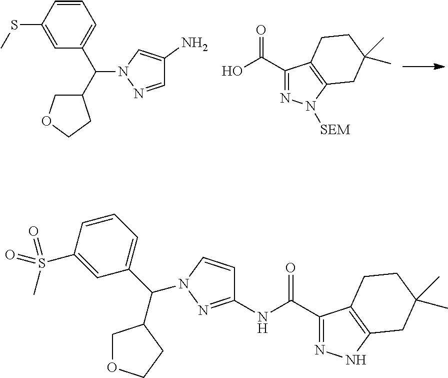 [SMILES: CSc1cccc(C(C2CCOC2)n2cc(N)cn2)c1 ; CC1(C)CCc2c(C(=O)O)nn(COCC[Si](C)(C)C)c2C1 ; CC1(C)CCc2c(C(=O)Nc3ccn(C(c4cccc(S(C)(=O)=O)c4)C4CCOC4)n3)n[nH]c2C1]

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with 1-((3-(methylthio)phenyl)(tetrahydrofuran-3-yl)methyl)-1H-pyrazol-4-amine (Example A68).

SFC conditions:

Diastereomer 1 (126a and 126b): Lux Cellulose 1 (4.6×50 mm, 5 μm particle size) at 40% methanol w/0.10% NH₄OH; 5 mL/min, 120 bars, 40° C.

Diastereomer 2 (126c and 126d): Lux Cellulose 4 (4.6×50 mm, 5 μm particle size) at 40% methanol w/0.10% NH₄OH; 5 mL/min, 120 bars, 40° C.

124a: ¹H NMR (400 MHz, DMSO-d₆) δ 12.89-12.70 (s, 1H), 10.14-10.03 (s, 1H), 8.28-8.22 (s, 1H), 8.08-8.04 (t, J=1.8 Hz, 1H), 7.92-7.83 (m, 2H), 7.70-7.67 (s, 1H), 7.67-7.62 (t, J=7.8 Hz, 1H), 5.48-5.29 (d, J=10.7 Hz, 1H), 3.85-3.78 (m, 1H), 3.73-3.65 (m, 1H), 3.56-3.43 (m, 2H), 3.27-3.22 (m, 1H), 3.22-3.20 (s, 3H), 2.70-2.63 (m, 2H), 2.41-2.37 (s, 2H), 1.94-1.78 (m, 1H), 1.58-1.49 (m, 1H), 1.49-1.44 (t, J=6.4 Hz, 2H), 1.00-0.93 (s, 6H); MS: m/z=498 (M+H); SFC retention time: 1.03 min.

124b: ¹H NMR (400 MHz, DMSO-d₆) δ 12.89-12.70 (s, 1H), 10.14-10.03 (s, 1H), 8.28-8.22 (s, 1H), 8.08-8.04 (t, J=1.8 Hz, 1H), 7.92-7.83 (m, 2H), 7.70-7.67 (s, 1H), 7.67-7.62 (t, J=7.8 Hz, 1H), 5.48-5.29 (d, J=10.7 Hz, 1H), 3.85-3.78 (m, 1H), 3.73-3.65 (m, 1H), 3.56-3.43 (m, 2H), 3.27-3.22 (m, 1H), 3.22-3.20 (s, 3H), 2.70-2.63 (m, 2H), 2.41-2.37 (s, 2H), 1.94-1.78 (m, 1H), 1.58-1.49 (m, 1H), 1.49-1.44 (t, J=6.4 Hz, 2H), 1.00-0.93 (s, 6H); MS: m/z=498 (M+H); SFC retention time: 0.92 min.

124c: ¹H NMR (400 MHz, DMSO-d₆) δ 12.85-12.64 (s, 1H), 10.23-10.03 (s, 1H), 8.21-8.16 (s, 1H), 8.10-8.07 (t, J=1.8 Hz, 1H), 7.92-7.85 (m, 2H), 7.70-7.63 (m, 2H), 5.50-5.37 (d, J=11.2 Hz, 1H), 3.87-3.77 (m, 1H), 3.69-3.60 (m, 2H), 3.53-3.40 (m, 1H), 3.24-3.19 (s, 3H), 2.69-2.62 (t, J=6.3 Hz, 2H), 2.41-2.36 (s, 2H), 1.80-1.66 (m, 1H), 1.52-1.39 (m, 4H), 0.99-0.92 (s, 6H); MS: m/z=498 (M+H); SFC retention time: 0.63 min.

124d: ¹H NMR (400 MHz, DMSO-d₆) δ 12.87-12.72 (s, 1H), 10.23-9.97 (s, 1H), 8.21-8.18 (s, 1H), 8.10-8.07 (t, J=1.9 Hz, 1H), 7.92-7.84 (m, 2H), 7.69-7.61 (m, 2H), 5.56-5.27 (d, J=11.2 Hz, 1H), 3.87-3.77 (m, 1H), 3.69-3.60 (m, 2H), 3.52-3.39 (m, 1H), 3.23-3.18 (s, 3H), 2.69-2.61 (t, J=6.5 Hz, 2H), 2.42-2.37 (s, 2H), 1.80-1.65 (m, 1H), 1.52-1.37 (m, 4H), 0.99-0.93 (s, 6H); MS: m/z=498 (M+H); SFC retention time: 0.52 min.

### Examples 127a and 127b

### N-(1-((1,1-dioxidothiomorpholin-2-yl)(phenyl)methyl)-1H-pyrazol-4-yl)-6,6-dimethyl-4,5,6,7-tetrahydro-1H-indazole-3-carboxamide

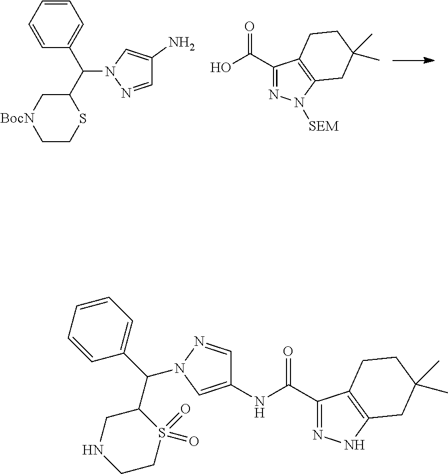 [SMILES: CC(C)(C)OC(=O)N1CCSC(C(c2ccccc2)n2cc(N)cn2)C1 ; CC1(C)CCc2c(C(=O)O)nn(COCC[Si](C)(C)C)c2C1 ; CC1(C)CCc2c(C(=O)Nc3cnn(C(c4ccccc4)C4CNCCS4(=O)=O)c3)n[nH]c2C1]

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with tert-butyl 2-((4-amino-1H-pyrazol-1-yl)(phenyl)methyl)thiomorpholine-4-carboxylate (Example A70). Note that only two of a possible four stereoisomers (one pair of enantiomers) were isolated.

SFC conditions: Lux Cellulose 1 (4.6×50 mm, 5 μm particle size) at 50% methanol w/0.1% NH₄OH; 5 mL/min, 120 bars, 40° C.

127a: ¹H NMR (400 MHz, DMSO-d₆) δ 12.81-12.68 (s, 1H), 10.12-10.01 (s, 1H), 8.20-8.16 (s, 1H), 7.66-7.63 (s, 1H), 7.57-7.52 (m, 2H), 7.39-7.27 (m, 3H), 5.90-5.77 (d, J=10.0 Hz, 1H), 4.40-4.31 (m, 1H), 3.24-2.69 (m, 6H), 2.69-2.63 (t, 2H), 2.39-2.36 (s, 2H), 1.50-1.42 (t, J=6.3 Hz, 2H), 0.98-0.93 (s, 6H); MS: m/z=483 (M+H); SFC retention time: 1.4 min.

127b: ¹H NMR (400 MHz, DMSO-d₆) δ 12.85-12.72 (s, 1H), 10.06-10.01 (s, 1H), 8.21-8.16 (s, 1H), 7.68-7.62 (s, 1H), 7.58-7.52 (m, 2H), 7.42-7.23 (m, 3H), 5.86-5.78 (d, J=10.0 Hz, 1H), 4.41-4.31 (m, 1H), 3.25-2.69 (m, 6H), 2.69-2.62 (t, 2H), 2.41-2.37 (s, 2H), 1.51-1.38 (t, J=6.4 Hz, 2H), 0.99-0.92 (s, 6H); MS: m/z=483 (M+H); SFC retention time: 1.7 min.

### Examples 128a and 128b

### N-(1-((1,1-dioxidothiomorpholin-3-yl)(phenyl)methyl)-1H-pyrazol-4-yl)-6,6-dimethyl-4,5,6,7-tetrahydro-1H-indazole-3-carboxamide

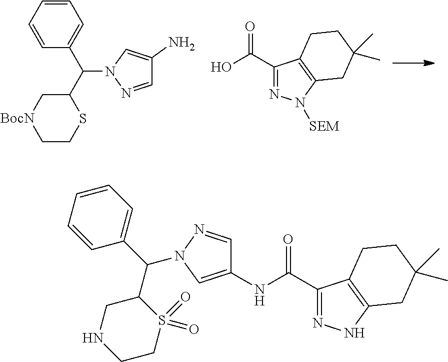 [SMILES: CC(C)(C)OC(=O)N1CCSC(C(c2ccccc2)n2cc(N)cn2)C1 ; CC1(C)CCc2c(C(=O)O)nn(COCC[Si](C)(C)C)c2C1 ; CC1(C)CCc2c(C(=O)Nc3cnn(C(c4ccccc4)C4CNCCS4(=O)=O)c3)n[nH]c2C1]

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with tert-butyl 3-((4-amino-1H-pyrazol-1-yl)(phenyl)methyl)thiomorpholine-4-carboxylate (Example A71). Note that only two of a possible four stereoisomers (one pair of enantiomers) were isolated.

SFC conditions: Chiralpak AS (4.6×50 mm, 5 μm particle size) at 35% methanol w/0.1% NH₄OH; 4 mL/min, 120 bars, 40° C.

128a: ¹H NMR (400 MHz, DMSO-d₆) δ 12.97-12.62 (s, 1H), 10.18-9.99 (s, 1H), 8.15-8.10 (s, 1H), 7.76-7.71 (s, 1H), 7.46-7.41 (m, 2H), 7.40-7.28 (m, 3H), 5.56-5.33 (d, J=8.2 Hz, 1H), 3.95-3.84 (m, 1H), 3.38-3.30 (dd, J=14.0, 2.7 Hz, 1H), 3.10-3.01 (m, 1H), 2.99-2.80 (m, 2H), 2.78-2.55 (m, 4H), 2.41-2.37 (s, 2H), 2.34-2.27 (m, 1H), 1.51-1.43 (t, J=6.3 Hz, 2H), 0.99-0.92 (s, 6H); MS: m/z=483 (M+H); SFC retention time: 0.45 min.

128b: ¹H NMR (400 MHz, DMSO-d₆) δ 12.87-12.68 (s, 1H), 10.16-9.99 (s, 1H), 8.15-8.10 (s, 1H), 7.76-7.72 (s, 1H), 7.46-7.41 (m, 2H), 7.40-7.28 (m, 3H), 5.50-5.29 (d, J=8.2 Hz, 1H), 3.96-3.81 (m, 1H), 3.40-3.30 (m, 1H), 3.09-3.00 (m, 1H), 3.00-2.81 (m, 2H), 2.77-2.55 (m, 4H), 2.42-2.37 (s, 2H), 2.34-2.27 (m, 1H), 1.50-1.43 (t, J=6.3 Hz, 2H), 0.99-0.92 (s, 6H); MS: m/z=483 (M+H); SFC retention time: 0.58 min.

### Examples 129a and 129b

### N-(1-((1,3-dimethyl-2-oxohexahydropyrimidin-5-yl)(phenyl)methyl)-1H-pyrazol-4-yl)-6,6-di methyl-4,5,6,7-tetrahydro-1H-indazole-3-carboxamide

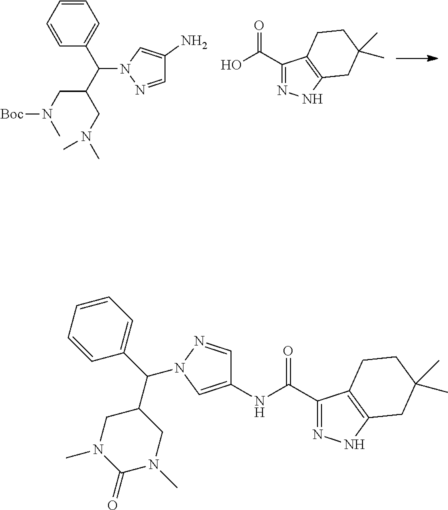

A solution of tert-butyl N-[2-[(4-amino-1H-pyrazol-1-yl)(phenyl)methyl]-3-[[(tert-butoxy)carbonyl](methyl)amino]propyl]-N-methylcarbamate (1.45 g, 3.06 mmol, 1.00 equiv; Example A82), 6,6-dimethyl-4,5,6,7-tetrahydro-1H-indazole-3-carboxylic acid (893 mg, 4.60 mmol, 1.50 equiv), DIEA (1.161 g, 8.98 mmol, 2.93 equiv), and HATU (1.748 g, 4.60 mmol, 1.50 equiv) in N,N-dimethylformamide (40 mL) was stirred for 4 h at room temperature. The reaction was diluted with 300 mL of ethyl acetate, washed with brine, dried over anhydrous sodium sulfate, and concentrated under vacuum. The residue was applied onto a silica gel column eluting with ethyl acetate/petroleum ether (1:1). This resulted in 1 g (50%) of tert-butyl N-(3-[[(tert-butoxy)carbonyl](methyl)amino]-2-[[4-(6,6-dimethyl-4,5,6,7-tetrahydro-1H-indazole-3-amido)-1H-pyrazol-1-yl](phenyl)methyl]propyl)-N-methylcarbamate as a yellow syrup.

A solution of tert-butyl N-(3-[[(tert-butoxy)carbonyl](methyl)amino]-2-[[4-(6,6-dimethyl-4,5,6,7-tetrahydro-1H-indazole-3-amido)-1H-pyrazol-1-yl](phenyl)methyl]propyl)-N-methylcarbamate (1 g, 1.54 mmol, 1.00 equiv) and trifluoroacetic acid (0.6 mL) in dichloromethane (50 mL) was stirred at room temperature overnight. The resulting solution was concentrated under vacuum. This resulted in 1 g (crude) of 6,6-dimethyl-N-[1-[3-(methylamino)-2-[(methylamino)methyl]-1-phenylpropyl]-1H-pyrazol-4-yl]-4,5,6,7-tetrahydro-1H-indazole-3-carboxamide as a brown syrup.

A solution of CDI (570 mg, 3.52 mmol, 1.51 equiv) and 6,6-dimethyl-N-[1-[3-(methylamino)-2-[(methylamino)methyl]-1-phenylpropyl]-1H-pyrazol-4-yl]-4,5,6,7-tetrahydro-1H-indazole-3-carboxamide (1.05 g, 2.34 mmol, 1.00 equiv) in dichloromethane (30 mL) was stirred at room temperature for 3 h. The resulting solution was concentrated under vacuum. The residue was applied onto a silica gel column eluting with ethyl acetate/petroleum ether (1:5) to give a crude product which was further purified by Prep-HPLC with the following conditions: Column, Xbridge Prep Phenyl OBD Column, 5 m, 19×150 mm, 10 mmol NH₄HCO₃ and CH₃CN (hold 76% CH₃CN in 15 min). The purified racemate was separated by Chiral-Prep-HPLC.

Chiral HPLC Conditions: ChiralPak IA (4.6×50 mm, 5 μm particle size); eluent=MTBE:IPA 90:10; 1.0 ml/min, 4.2 MPA, 25° C.

129a: ¹H NMR (300 MHz, CD₃OD) δ 8.16 (s, 1H), 7.72 (s, 1H), 7.57-7.55 (m, 2H), 7.50-7.38 (m, 3H), 5.28 (d, J=11.1 Hz, 1H), 3.25-3.05 (m, 3H), 3.00-2.90 (m, 2H), 2.85 (s, 3H), 2.78-2.70 (m, 5H), 2.43 (s, 2H), 1.06-1.04 (m, 2H), 1.03 (s, 6H); MS: m/z=476 (M+H); HPLC retention time: 23.1 min.

129b: ¹H NMR (300 MHz, CD₃OD) δ 8.16 (s, 1H), 7.72 (s, 1H), 7.65-7.55 (m, 2H), 7.45-7.35 (m, 3H), 5.30 (d, J=30 Hz, 1H), 3.25-3.05 (m, 2H), 3.05-2.90 (m, 2H), 2.85 (s, 3H), 2.81-2.75 (m, 5H), 2.43 (s, 2H), 1.61-1.50 (m, 2H), 1.03 (s, 6H); MS: m/z=476 (M+H); HPLC retention time: 32.1 min.

### Examples 130a and 130b

### N-(1-benzyl-1H-pyrazol-4-yl)-6-ethynyl-6-methyl-4,5,6,7-tetrahydro-1H-indazole-3-carboxamide

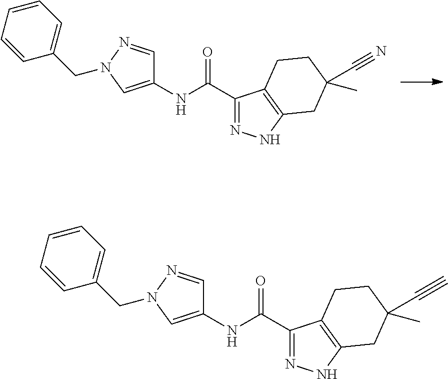 [SMILES: CC1(C#N)CCc2c(C(=O)Nc3cnn(Cc4ccccc4)c3)n[nH]c2C1 ; C#CC1(C)CCc2c(C(=O)Nc3cnn(Cc4ccccc4)c3)n[nH]c2C1]

Under nitrogen DIBAL-H (40 mL, 1M in toluene, 7.00 equiv) was added dropwise into a solution of N-(1-benzyl-1H-pyrazol-4-yl)-6-cyano-6-methyl-4,5,6,7-tetrahydro-1H-indazole-3-carboxamide (2 g, 5.55 mmol, 1.00 equiv; Example 102) in toluene (200 mL) at −78° C. After 2 h at −78° C. the reaction was quenched by saturated NH₄Cl, extracted with ethyl acetate, and concentrated under vacuum. The residue was applied onto a silica gel column eluting with ethyl acetate/petroleum ether (15:85). This resulted in 800 mg (40%) of N-(1-benzyl-1H-pyrazol-4-yl)-6-formyl-6-methyl-4,5,6,7-tetrahydro-1H-indazole-3-carboxamide as a yellow solid.

A mixture of N-(1-benzyl-1H-pyrazol-4-yl)-6-formyl-6-methyl-4,5,6,7-tetrahydro-1H-indazole-3-carboxamide (300 mg, 0.83 mmol, 1.00 equiv), (dimethoxyphosphoryl)methanediazonium (185.9 mg, 1.24 mmol, 1.50 equiv), Cs₂CO₃ (807 mg, 2.48 mmol, 3.00 equiv) in methanol (10 mL) was stirred at room temperature overnight. The reaction was quenched by 50 mL of water, extracted with ethyl acetate, and concentrated under vacuum. The enantiomers were separated by preparative chiral HPLC.

Chiral HPLC Conditions: ChiralCel OJ (4.6×150 mm, 3 μm particle size); eluent=Hex:EtOH 60:40; 1.0 ml/min, 4.2 MPA, 25° C.

130a: ¹H NMR (300 MHz, CD₃OD) δ 7.94 (s, 1H), 7.58 (s, 1H), 7.26-7.14 (m, 5H), 5.21 (s, 1H), 3.22-3.20 (m, 3H), 2.84-2.76 (m, 1H), 2.25 (s, 1H), 1.86-1.80 (m, 1H), 1.60-1.52 (m, 1H), 1.28 (s, 3H); MS: m/z=360 (M+H); HPLC retention time: 5.1 min.

130b: ¹H NMR (300 MHz, CD₃OD) δ 8.06 (s, 1H), 7.70 (s, 1H), 7.39-7.26 (m, 5H), 5.33 (s, 1H), 2.93-2.88 (m, 3H), 2.64-2.60 (m, 1H), 2.37 (s, 1H), 1.97-1.92 (m, 1H), 1.72-1.64 (m, 1H), 1.40 (s, 3H); MS: m/z=360 (M+H); HPLC retention time: 8.0 min.

### Examples 131a and 131b

### 6,6-dimethyl-N-(1-(2-(3-methyl-2-oxoimidazolidin-1-yl)-1-phenylethyl)-1H-pyrazol-4-yl)-4,5,6,7-tetrahydro-1H-indazole-3-carboxamide

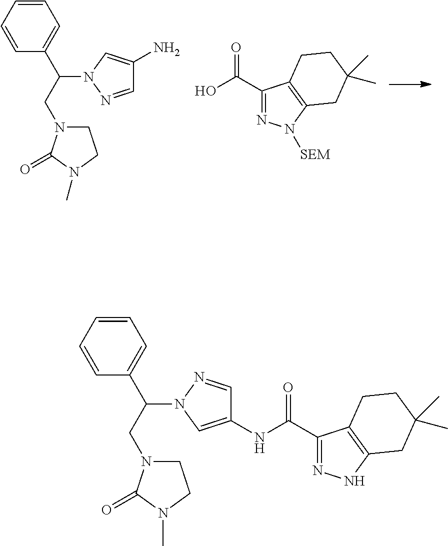 [SMILES: CN1CCN(CC(c2ccccc2)n2cc(N)cn2)C1=O ; CC1(C)CCc2c(C(=O)O)nn(COCC[Si](C)(C)C)c2C1 ; CN1CCN(CC(c2ccccc2)n2cc(NC(=O)c3n[nH]c4c3CCC(C)(C)C4)cn2)C1=O]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 1-(2-(4-amino-1H-pyrazol-1-yl)-2-phenylethyl)-3-methylimidazolidin-2-one (Example A72). Also, the stereoisomers were separated by preparative chiral HPLC instead of SFC.

Chiral HPLC Conditions: Lux Cellulose-4 (4.6×150 mm, 3 μm particle size); eluent=Hex:EtOH 60:40; 1.0 ml/min, 4.2 MPA, 25° C.

131a: ¹H NMR (300 MHz, DMSO-d₆) δ 12.80 (s, 1H), 10.11 (s, 1H), 8.15 (s, 1H), 7.70 (s, 1H), 7.41-7.27 (m, 5H), 5.59-5.54 (m, 1H), 3.95-3.77 (m, 2H), 3.32-3.06 (m, 3H), 2.90-2.83 (m, 1H), 2.68-2.61 (m, 5H), 2.38 (s, 2H), 1.49 (s, 2H), 1.00-0.90 (s, 6H); MS: m/z=462 (M+H); HPLC retention time: 6.5 min.

131b: ¹H NMR (300 MHz, DMSO-d₆) δ 12.80 (s, 1H), 10.10 (s, 1H), 8.15 (s, 1H), 7.70 (s, 1H), 7.41-7.27 (m,H), 5.59-5.54 (m, 1H), 3.95-3.77 (m, 2H), 3.31-3.06 (m, 3H), 2.90-2.83 (m, 1H), 2.68-2.61 (m, 5H), 2.38 (s, 2H), 1.49 (s, 2H), 0.90-1.00 (s, 6H); MS: m/z=462 (M+H); HPLC retention time: 8.4 min.

### Examples 132a and 132b

### 6,6-dimethyl-N-(1-(4-phenyltetrahydrofuran-3-yl)-1H-pyrazol-4-yl)-4,5,6,7-tetrahydro-1H-indazole-3-carboxamide

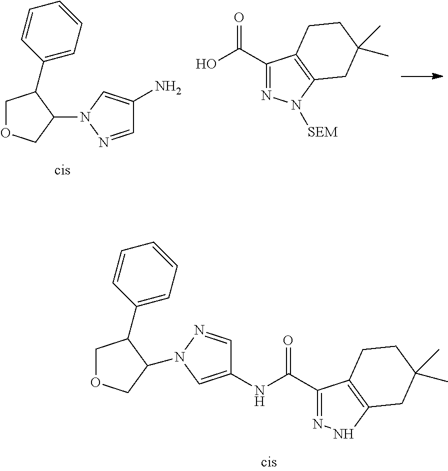 [SMILES: Nc1cnn(C2COCC2c2ccccc2)c1 ; CC1(C)CCc2c(C(=O)O)nn(COCC[Si](C)(C)C)c2C1 ; CC1(C)CCc2c(C(=O)Nc3cnn(C4COCC4c4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with cis-1-(4-phenyltetrahydrofuran-3-yl)-1H-pyrazol-4-amine (Example A74). Also, the stereoisomers were separated by preparative chiral HPLC instead of SFC.

Chiral HPLC Conditions: ChiralPak IA-3 (4.6×50 mm, 3 μm particle size); eluent=Hex:IPA 70:30; 1.0 ml/min, 4.2 MPA, 25° C.

132a: ¹H NMR (400 MHz, CD₃OD) δ 7.81 (s, 1H), 7.53 (s, 1H), 7.16-7.12 (s, 3H), 7.11-6.94 (m, 2H), 5.15-5.11 (s, 1H), 4.49-4.27 (m, 4H), 3.91-3.86 (m, 1H), 2.79-2.76 (t, J=6.0 Hz, 2H), 2.44 (s, 2H), 1.59-1.56 (t, J=2.0 Hz, 2H), 1.04 (s, 6H); MS: m/z=405 (M+H); HPLC retention time: 2.8 min.

132b: ¹H NMR (400 MHz, CD₃OD) δ 7.81 (s, 1H), 7.53 (s, 1H), 7.13-7.12 (m, 3H), 6.96-6.94 (m, 2H), 5.15-5.12 (m, 1H), 4.45-4.28 (m, 4H), 3.92-3.86 (m, 1H), 2.79-2.76 (t, J=6.0 Hz, 2H), 2.44 (s, 2H), 1.59-1.56 (t, J=2.0 Hz, 2H), 1.04 (s, 6H); MS: m/z=405 (M+H); HPLC retention time: 6.8 min.

### Examples 133a and 133b

### N-(1-((2-aminopyridin-4-yl)(tetrahydro-2H-pyran-4-yl)methyl)-1H-pyrazol-4-yl)-6,6-dimethyl-4,5,6,7-tetrahydro-1H-indazole-3-carboxamide

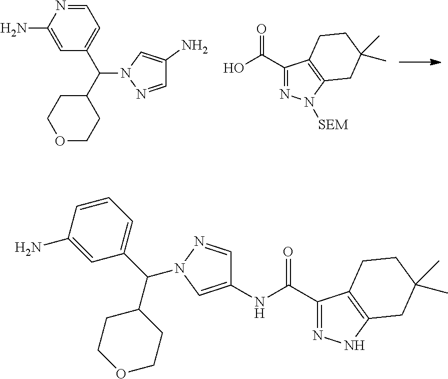 [SMILES: Nc1cnn(C(c2ccnc(N)c2)C2CCOCC2)c1 ; CC1(C)CCc2c(C(=O)O)nn(COCC[Si](C)(C)C)c2C1 ; CC1(C)CCc2c(C(=O)Nc3cnn(C(c4cccc(N)c4)C4CCOCC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 4-((4-amino-1H-pyrazol-1-yl)(tetrahydro-2H-pyran-4-yl)methyl)pyridin-2-amine (Example A75). Also, the stereoisomers were separated by preparative chiral HPLC instead of SFC.

Chiral HPLC Conditions: ChiralPak IA (4.6×50 mm, 3 μm particle size); eluent=Hex:IPA 70:30; 1.0 ml/min, 4.2 MPA, 25° C.

133a: ¹H NMR (300 MHz, CDCl₃) δ 8.59 (s, 1H), 8.15 (s, 1H), 7.92 (d, J=6.0 Hz, 1H), 7.53 (s, 1H), 6.73 (d, J=5.1 Hz, 1H), 6.64 (s, 1H), 5.18-5.12 (m, 1H), 4.60 (d, J=10.5 Hz, 1H), 3.92 (d, J=10.5 Hz, 1H), 3.42-3.29 (m, 2H), 2.86-2.82 (m, 2H), 2.62-2.53 (m, 1H), 1.58-1.54 (m, 2H), 1.37-1.22 (m, 4H), 1.01 (s, 6H); MS: m/z=450 (M+H); HPLC retention time: 1.9 min.

133b: ¹H NMR (300 MHz, CDCl₃) δ 8.62 (s, 1H), 8.59 (s, 1H), 8.15 (s, 1H), 7.88 (d, J=5.4 Hz, 1H), 7.54 (s, 1H), 6.76-6.70 (m, 2H), 5.53 (s, 1H), 4.60 (d, J=11.4 Hz, 1H), 3.92 (d, J=11.4 Hz, 1H), 3.40-3.29 (m, 2H), 2.86-2.81 (m, 2H), 2.63-2.58 (m, 2H), 2.42 (s, 1H), 1.58-1.54 (m, 2H), 1.47-1.19 (m, 4H), 1.01 (s, 6H); MS: m/z=450 (M+H); HPLC retention time: 3.2 min.

### Examples 134a-d

### N-(1-((1,1-dioxidotetrahydro-2H-thiopyran-2-yl)(phenyl)methyl)-1H-pyrazol-4-yl)-6,6-dimethyl-4,5,6,7-tetrahydro-1H-indazole-3-carboxamide

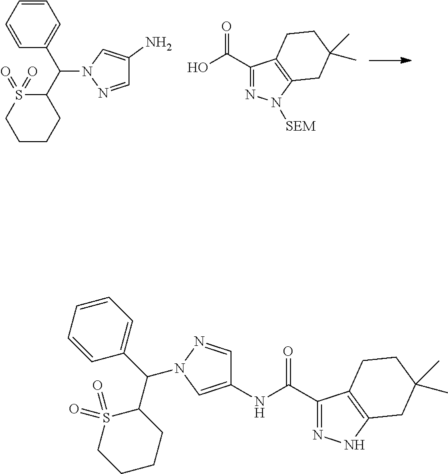 [SMILES: Nc1cnn(C(c2ccccc2)C2CCCCS2(=O)=O)c1 ; CC1(C)CCc2c(C(=O)O)nn(COCC[Si](C)(C)C)c2C1 ; CC1(C)CCc2c(C(=O)Nc3cnn(C(c4ccccc4)C4CCCCS4(=O)=O)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 2-((4-amino-1H-pyrazol-1-yl)(phenyl)methyl)tetrahydro-2H-thiopyran 1,1-dioxide (Example A76). Also, the stereoisomers were separated by preparative chiral HPLC instead of SFC.

Chiral HPLC Conditions: ChiralPak IB-3 (4.6×150 mm, 3 μm particle size); eluent=Hex:EtOH 80:20; 1.0 ml/min, 4.2 MPA, 25° C.

134a: ¹H NMR (300 MHz, CDCl₃) δ 8.68 (s, 1H), 8.12 (s, 1H), 7.62-7.57 (m, 3H), 7.37-7.30 (m, 3H), 5.55 (d, J=9.6 Hz, 1H), 4.13-4.05 (m, 2H), 3.06-2.82 (m, 4H), 2.43 (s, 2H), 2.04-2.02 (m, 2H), 1.89-1.70 (m, 3H), 1.59-1.50 (m, 3H), 1.01 (s, 6H); MS: m/z=482 (M+H); HPLC retention time: 1.4 min.

134b: ¹H NMR: (300 MHz, CDCl₃) δ 9.06 (s, 1H), 8.12 (s, 1H), 7.69 (s, 1H), 7.61-7.58 (m, 2H), 7.37-7.30 (m, 3H), 5.56 (d, J=9.6 Hz, 1H), 4.14-4.06 (m, 2H), 3.06-2.85 (m, 4H), 2.49 (s, 2H), 2.05-2.00 (m, 2H), 1.87-1.71 (m, 3H), 1.61-1.45 (m, 3H), 1.01 (s, 6H); MS: m/z=482 (M+H); HPLC retention time: 2.2 min.

134c: ¹H NMR: (300 MHz, CDCl₃) δ 8.78 (1H, s), 8.08 (s, 1H), 7.66 (s, 1H), 7.44-7.42 (m, 2H), 7.32-7.30 (m, 3H), 5.73 (d, J=9.6 Hz, 1H), 4.43-4.35 (m, 1H), 3.11-3.00 (m, 4H), 2.82-2.78 (m, 4H), 2.40 (s, 2H), 2.03-1.98 (m, 2H), 1.84-1.70 (m, 2H), 1.64-1.43 (m, 4H), 0.99 (s, 6H); MS: m/z=482 (M+H); HPLC retention time: 7.3 min.

134d: ¹H NMR (300 MHz, CDCl₃) δ 8.91 (s, 1H), 8.06 (s, 1H), 7.63 (s, 1H), 7.44-7.41 (m, 2H), 7.32-7.26 (m, 3H), 5.71 (d, J=8.7 Hz, 1H), 4.43-4.35 (m, 1H), 3.11-2.95 (m, 4H), 2.82-2.70 (m, 2H), 2.40 (s, 2H), 2.03-1.98 (m, 2H), 1.84-1.70 (m, 2H), 1.64-1.47 (m, 4H), 0.99 (s, 6H); MS: m/z=482 (M+H); HPLC retention time: 5.7 min.

### Examples 135a and 135b

### 6,6-dimethyl-N-(1-(4-phenyltetrahydrofuran-3-yl)-1H-pyrazol-4-yl)-4,5,6,7-tetrahydro-1H-indazole-3-carboxamide

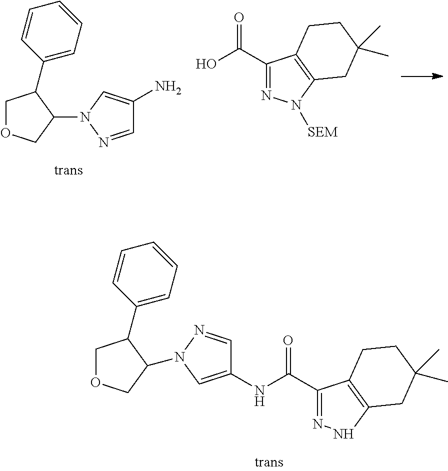 [SMILES: Nc1cnn(C2COCC2c2ccccc2)c1 ; CC1(C)CCc2c(C(=O)O)nn(COCC[Si](C)(C)C)c2C1 ; CC1(C)CCc2c(C(=O)Nc3cnn(C4COCC4c4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with trans-1-(4-phenyltetrahydrofuran-3-yl)-1H-pyrazol-4-amine (Example A73). Also, the stereoisomers were separated by preparative chiral HPLC instead of SFC.

Chiral HPLC Conditions: ChiralPak IA-3 (4.6×50 mm, 3 μm particle size); eluent=Hex:IPA 70:30; 1.0 ml/min, 4.2 MPA, 25° C.

135a: ¹H NMR (CD₃OD, 300 MHz) δ 7.80 (s, 1H), 7.52 (s, 1H), 7.13-7.10 (m, 3H), 6.96-6.92 (m, 2H), 5.15-5.10 (m, 1H), 4.49-4.26 (m, 4H), 3.92-3.86 (m, 1H), 2.78-2.74 (t, J=6.0 Hz, 2H), 2.43 (s, 2H), 1.56 (t, J=6.0 Hz, 2H), 1.00 (s, 6H); MS: m/z=405 (M+H); HPLC retention time: 2.8 min.

135b: ¹H NMR (CD₃OD, 300 MHz) δ 7.80 (s, 1H), 7.52 (s, 1H), 7.13-7.10 (m, 3H), 6.96-6.92 (m, 2H), 5.15-5.10 (m, 1H), 4.49-4.26 (m, 4H), 3.92-3.83 (m, 1H), 2.76 (t, J=6.0 Hz, 2H), 2.43 (s, 2H), 1.56 (t, J=6.0 Hz, 2H), 1.00 (s, 6H); MS: m/z=405 (M+H); HPLC retention time: 7.3 min.

### Examples 136a-d

### N-(1-((1,1-dioxidotetrahydrothiophen-3-yl)(phenyl)methyl)-1H-pyrazol-4-yl)-6,6-dimethyl-4,5,6,7-tetrahydro-1H-indazole-3-carboxamide

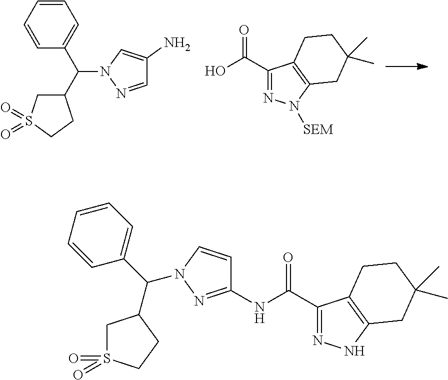 [SMILES: Nc1cnn(C(c2ccccc2)C2CCS(=O)(=O)C2)c1 ; CC1(C)CCc2c(C(=O)O)nn(COCC[Si](C)(C)C)c2C1 ; CC1(C)CCc2c(C(=O)Nc3ccn(C(c4ccccc4)C4CCS(=O)(=O)C4)n3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 3-((4-amino-1H-pyrazol-1-yl)(phenyl)methyl)tetrahydrothiophene 1,1-dioxide (Example A78). Also, the stereoisomers were separated by preparative chiral HPLC instead of SFC.

Chiral HPLC Conditions: Lux Cellulose-4 (4.6×150 mm, 3 μm particle size); eluent=Hex:EtOH 60:40; 1.0 ml/min, 4.2 MPA, 25° C.

136a: ¹H NMR (300 MHz, CDCl₃) δ 8.62 (s, 1H), 8.14 (s, 1H), 7.57 (s, 1H), 7.43-7.31 (m, 5H), 5.06 (d, J=10.5 Hz, 1H), 3.69-3.66 (m, 1H), 3.24-2.98 (m, 3H), 2.86-2.82 (t, J=6.3 Hz, 2H), 2.78-2.70 (m, 1H), 2.43 (s, 2H), 2.24-2.22 (m, 1H), 2.01-1.94 (m, 1H), 1.59-1.55 (t, J=6.3 Hz, 2H), 1.02 (s, 6H); MS: m/z=468 (M+H); HPLC retention time: 12.3 min.

136b: ¹H NMR (300 MHz, CDCl₃) δ 8.68 (s, 1H), 8.10 (s, 1H), 7.57 (s, 1H), 7.40-7.32 (m, 5H), 5.04 (d, J=10.5 Hz, 1H), 3.67-3.65 (m, 1H), 3.32-2.91 (m, 4H), 2.86 (t, J=6 Hz, 2H), 2.44 (s, 2H), 2.02-1.86 (m, 2H), 1.59-1.55 (t, J=6.3 Hz, 2H), 1.02 (s, 6H); MS: m/z=468 (M+H); HPLC retention time: 15.1 min.

136c: ¹H NMR (300 MHz, CDCl₃) δ 8.66 (s, 1H), 8.14 (s, 1H), 7.57 (s, 1H), 7.43-7.31 (m, 5H), 5.06 (d, J=10.5 Hz, 1H), 3.72-3.64 (m, 1H), 3.28-2.98 (m, 3H), 2.84 (t, J=6.3 Hz, 2H), 2.78-2.70 (m, 1H), 2.43 (s, 2H), 2.24-2.21 (m, 1H), 2.01-1.94 (m, 1H), 1.57 (t, J=6.3 Hz, 2H), 1.02 (s, 6H); MS: m/z=468 (M+H); HPLC retention time: 18.4 min.

136d: ¹H NMR (300 MHz, CDCl₃) δ 8.62 (s, 1H), 8.10 (s, 1H), 7.55 (s, 1H), 7.37-7.31 (m, 5H), 5.03 (d, J=10.5 Hz. 1H), 3.67-3.61 (m, 1H), 3.32-2.88 (m, 4H), 2.83 (t, J=6.3 Hz, 2H), 2.42 (s, 2H), 2.02-1.86 (m, 2H), 1.57 (t, J=6.3 Hz, 2H), 1.02 (s, 6H); MS: m/z=468 (M+H); HPLC retention time: 24.4 min.

### Examples 137a-d

### N-(1-((1,1-dioxidotetrahydro-2H-thiopyran-3-yl)(phenyl)methyl)-1H-pyrazol-4-yl)-6,6-dimethyl-4,5,6,7-tetrahydro-1H-indazole-3-carboxamide

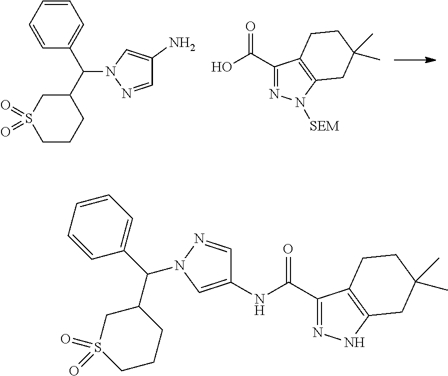 [SMILES: Nc1cnn(C(c2ccccc2)C2CCCS(=O)(=O)C2)c1 ; CC1(C)CCc2c(C(=O)O)nn(COCC[Si](C)(C)C)c2C1 ; CC1(C)CCc2c(C(=O)Nc3cnn(C(c4ccccc4)C4CCCS(=O)(=O)C4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 3-((4-amino-1H-pyrazol-1-yl)(phenyl)methyl)tetrahydro-2H-thiopyran 1,1-dioxide (Example A77). Also, the stereoisomers were separated by preparative chiral HPLC instead of SFC.

Chiral HPLC Conditions: ChiralPak IA (4.6×250 mm, 3 μm particle size); eluent=(Hex+0.1% Et₃N):EtOH 50:50; 1.0 ml/min, 4.2 MPA, 25° C.

137a: ¹H NMR (300 MHz, CDCl₃) δ 9.00 (s, 1H), 8.14 (s, 1H), 7.61 (s, 1H), 7.44-7.29 (m, 5H), 4.99 (d, J=9.3 Hz, 1H), 3.35 (d, J=4.5 Hz, 1H), 3.08-3.02 (m, 1H), 2.93-2.84 (m, 4H), 2.72-2.58 (m, 1H), 2.58 (s, 2H), 2.13-2.05 (m, 2H), 2.01 (s, 1H), 1.72-1.67 (m, 1H), 1.59 (t, J=7.5 Hz, 2H), 1.32-1.26 (m, 1H), 1.03 (s, 6H); MS: m/z=482 (M+H); HPLC retention time: 15.5 min.

137b: ¹H NMR (300 MHz, CDCl₃) δ 9.50 (s, 1H), 8.13 (s, 1H), 7.69 (s, 1H), 7.44-7.42 (m, 5H), 5.01 (d, J=9.3 Hz, 1H), 3.35 (d, J=6.0 Hz, 1H), 3.07-3.03 (m, 1H), 2.92-2.83 (m, 4H), 2.72-2.59 (m, 1H), 2.5 (s, 2H), 2.13-2.06 (m, 3H), 1.85-1.57 (m, 5H), 1.34-1.25 (m, 2H), 1.03 (s, 6H); MS: m/z=482 (M+H); HPLC retention time: 22.8 min.

137c: ¹H NMR (300 MHz, CDCl₃) δ 8.92 (s, 1H), 8.11 (s, 1H), 7.68 (s, 1H), 7.39-7.29 (m, 5H), 5.07 (d, J=9.3 Hz, 1H), 3.27-3.21 (m, 1H), 3.06-2.84 (m, 6H), 2.72 (s, 2H), 2.11-2.03 (m, 2H), 1.77-1.72 (m, 1H), 1.55 (t, J=7.5 Hz, 2H), 1.25-1.20 (m, 1H), 1.03 (s, 6H); MS: m/z=482 (M+H); HPLC retention time: 13.2 min.

137d: ¹H NMR (300 MHz, CDCl₃) δ 8.87 (s, 1H), 8.11 (s, 1H), 7.62 (s, 1H), 7.39-7.29 (m, 5H), 5.06 (d, J=9.3 Hz, 1H), 3.28-3.21 (m, 1H), 3.06-2.83 (m, 6H), 2.46 (s, 2H), 2.11-2.04 (m, 2H), 1.77-1.72 (m, 1H), 1.58 (t, J=7.5 Hz, 2H), 1.25-1.20 (m, 1H), 1.03 (s, 6H); MS: m/z=482 (M+H); HPLC retention time: 18.7 min.

### Examples 138a and 138b

### N-(1-benzyl-1H-pyrazol-4-yl)-5,5-difluoro-5a-methyl-1,4,4a,5,5a,6-hexahydrocyclopropa[f]indazole-3-carboxamide

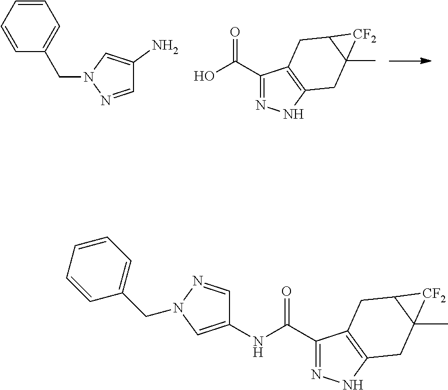 [SMILES: Nc1cnn(Cc2ccccc2)c1 ; CC12Cc3[nH]nc(C(=O)O)c3CC1C2(F)F ; CC12Cc3[nH]nc(C(=O)Nc4cnn(Cc5ccccc5)c4)c3CC1C2(F)F]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5,5-difluoro-5a-methyl-1,4,4a,5,5a,6-hexahydrocyclopropa[f]indazole-3-carboxylic acid (Example C37) and 3-((4-amino-1H-pyrazol-1-yl)methyl)benzonitrile (Example A1) with 1-benzyl-1H-pyrazol-4-amine (Example A2), and removing the SEM-deprotection step. Also, the stereoisomers were separated by preparative chiral HPLC instead of SFC.

Chiral HPLC Conditions: ChiralPak IA-3 (4.6×50 mm, 3 μm particle size); eluent=(Hex+0.1% Et₂NH):EtOH 50:50; 1.0 ml/min, 4.2 MPA, 25° C.

138a: ¹H NMR (300 MHz, CDCl₃) δ 8.75 (s, 1H), 8.03 (s, 1H), 7.60 (s, 1H), 7.38-7.22 (m, 5H), 5.29 (s, 2H), 3.37-3.02 (m, 3H), 2.88-2.74 (m, 1H), 1.66-1.59 (m, 1H), 1.41 (s, 3H); MS: m/z=384 (M+H); HPLC retention time: 1.4 min.

138b: ¹H NMR (300 MHz, CDCl₃) δ 8.67 (s, 1H), 8.03 (s, 1H), 7.61 (s, 1H), 7.37-7.24 (m, 5H), 5.29 (s, 2H), 3.34-3.03 (m, 3H), 2.80-2.71 (m, 1H), 1.66-1.58 (m, 1H), 1.40 (s, 3H); MS: m/z=384 (M+H); HPLC retention time: 2.8 min.

### Examples 139a-d

### 6,6-dimethyl-N-(1-((2-methyl-1,1-dioxidotetrahydro-2H-thiopyran-4-yl)(phenyl)methyl)-1H-pyrazol-4-yl)-4,5,6,7-tetrahydro-1H-indazole-3-carboxamide

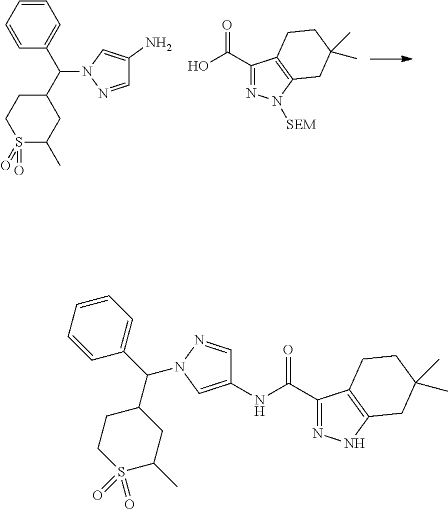 [SMILES: CC1CC(C(c2ccccc2)n2cc(N)cn2)CCS1(=O)=O ; CC1(C)CCc2c(C(=O)O)nn(COCC[Si](C)(C)C)c2C1 ; CC1CC(C(c2ccccc2)n2cc(NC(=O)c3n[nH]c4c3CCC(C)(C)C4)cn2)CCS1(=O)=O]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 4-((4-amino-1H-pyrazol-1-yl)(phenyl)methyl)-2-methyltetrahydro-2H-thiopyran 1,1-dioxide (Example A79a). Also, the stereoisomers were separated by preparative chiral HPLC instead of SFC.

Chiral HPLC Conditions: ChiralPak IC (4.6×250 mm, 3 μm particle size); eluent=(Hex+0.1% Et₃N):EtOH 60:40; 1.0 ml/min, 4.2 MPA, 25° C.

139a: ¹H NMR (300 MHz, CD₃OD) δ 8.16 (s, 1H), 7.69 (s, 1H), 7.61-7.58 (m, 2H), 7.41-7.32 (m, 3H), 5.37 (d, J=11.4 Hz, 2H), 3.10-3.03 (m, 3H), 2.80-2.76 (m, 2H), 2.43 (s, 2H), 2.05-1.73 (m, 4H), 1.59-1.54 (m, 2H), 1.36-1.33 (m, 3H), 1.01 (s, 6H); MS: m/z=465 (M+H); HPLC retention time: 10.0 min.

139b: ¹H NMR (300 MHz, CD₃OD) δ 8.17 (s, 1H), 7.69 (s, 1H), 7.59-7.57 (m, 2H), 7.42-7.33 (m, 3H), 5.37 (d, J=11.4 Hz, 2H), 3.10-3.05 (m, 4H), 2.80-2.76 (m, 2H), 2.43 (s, 2H), 2.04-1.73 (m, 4H), 1.59-1.54 (m, 2H), 1.36-1.33 (m, 3H), 1.01 (s, 6H); MS: m/z=465 (M+H); HPLC retention time: 14.3 min.

139c: ¹H NMR (300 MHz, CD₃OD) δ 8.17 (s, 1H), 7.69 (s, 1H), 7.59-7.57 (m, 2H), 7.42-7.33 (m, 3H), 5.37 (d, J=11.4 Hz, 2H), 3.10-3.05 (m, 4H), 2.80-2.76 (m, 2H), 2.43 (s, 2H), 2.04-1.73 (m, 4H), 1.59-1.54 (m, 2H), 1.36-1.33 (m, 3H), 1.01 (s, 6H); MS: m/z=465 (M+H); HPLC retention time: 18.4 min.

139d: ¹H NMR (300 MHz, CD₃OD) δ 8.16 (s, 1H), 7.69 (s, 1H), 7.61-7.58 (m, 2H), 7.41-7.32 (m, 3H), 5.37 (d, J=11.4 Hz, 2H), 3.10-3.06 (m, 3H), 2.80-2.76 (m, 2H), 2.43 (s, 2H), 2.05-1.73 (m, 4H), 1.59-1.55 (m, 2H), 1.36-1.33 (m, 3H), 1.03 (s, 6H); MS: m/z=465 (M+H); HPLC retention time: 23.6 min.

### Examples 139e-h

### 6,6-dimethyl-N-(1-((2-methyl-1,1-dioxidotetrahydro-2H-thiopyran-4-yl)(phenyl)methyl)-1H-pyrazol-4-yl)-4,5,6,7-tetrahydro-1H-indazole-3-carboxamide

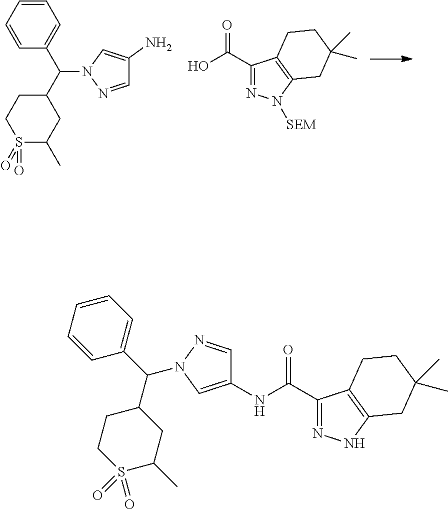 [SMILES: CC1CC(C(c2ccccc2)n2cc(N)cn2)CCS1(=O)=O ; CC1(C)CCc2c(C(=O)O)nn(COCC[Si](C)(C)C)c2C1 ; CC1CC(C(c2ccccc2)n2cc(NC(=O)c3n[nH]c4c3CCC(C)(C)C4)cn2)CCS1(=O)=O]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 4-((4-amino-1H-pyrazol-1-yl)(phenyl)methyl)-2-methyltetrahydro-2H-thiopyran 1,1-dioxide (Example A79b). Also, the stereoisomers were separated by preparative chiral HPLC instead of SFC.

Chiral HPLC Conditions: ChiralPak AD-H (4.6×150 mm, 3 μm particle size); eluent=(Hex+0.1% Et₃N):EtOH 50:50; 1.0 ml/min, 4.2 MPA, 25° C.

139e: ¹H NMR (300 MHz, CD₃OD) δ 8.17 (s, 1H), 7.69 (s, 1H), 7.55-7.52 (m, 2H), 7.39-7.31 (m, 3H), 5.05 (d, J=10.8 Hz, 1H), 3.24-3.12 (m, 2H), 3.09-2.80 (m, 2H), 2.78-2.76 (m, 2H), 2.43 (s, 2H), 1.81-1.73 (m, 2H), 1.69-1.54 (m, 4H), 1.21-1.19 (m, 3H), 1.03 (s, 6H); MS: m/z=465 (M+H); HPLC retention time: 7.7 min.

139f: ¹H NMR (300 MHz, CD₃OD) δ 8.17 (s, 1H), 7.68 (s, 1H), 7.55-7.52 (m, 2H), 7.40-7.31 (m, 3H), 5.05 (d, J=10.8 Hz, 1H), 3.31-3.02 (m, 3H), 2.94-2.80 (m, 1H), 2.78-2.76 (m, 2H), 2.43 (s, 2H), 1.87-1.70 (m, 1H), 1.63-1.61 (m, 1H), 1.58-1.52 (m, 4H), 1.16-1.14 (m, 3H), 1.02 (s, 6H); MS: m/z=465 (M+H); HPLC retention time: 12.9 min.

139g: ¹H NMR (300 MHz, CD₃OD) δ 8.13 (s, 1H), 7.69 (s, 1H), 7.55-7.52 (m, 2H), 7.40-7.29 (m, 3H), 5.06 (d, J=10.8 Hz, 1H), 3.26-3.10 (m, 2H), 2.90-2.80 (m, 2H), 2.80-2.76 (m, 2H), 2.43 (s, 2H), 1.81-1.75 (m, 2H), 1.70-1.61 (m, 2H), 1.58-1.52 (m, 2H), 1.21-1.20 (m, 3H), 1.02 (s, 6H); MS: m/z=465 (M+H); HPLC retention time: 16.5 min.

139h: ¹H NMR (300 MHz, CD₃OD) δ 8.13 (s, 1H), 7.69 (s, 1H), 7.56-7.52 (m, 2H), 7.41-7.32 (m, 3H), 5.07-5.04 (d, J=10.8 Hz, 1H), 3.21-3.03 (m, 3H), 2.94-2.80 (m, 1H), 2.80-2.27 (m, 2H), 2.43 (s, 2H), 1.92-1.87 (m, 1H), 1.76-1.61 (m, 1H), 1.59-1.54 (m, 4H), 1.17-1.15 (m, 3H), 1.03 (s, 6H); MS: m/z=465 (M+H); HPLC retention time: 21.4 min.

### Examples 140a and 140b

### 4-((4-(6,6-dimethyl-4,5,6,7-tetrahydro-1H-indazole-3-carboxamido)-1H-pyrazol-1-yl)(phenyl)methyl)-1-methylpiperidine 1-oxide

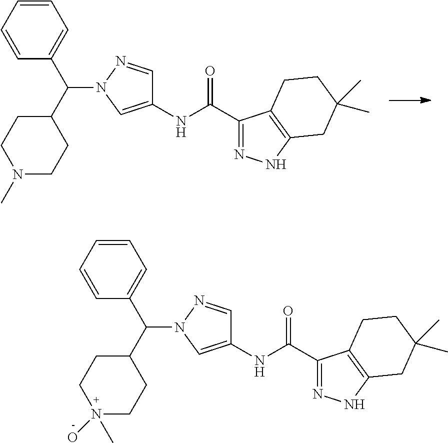

A solution of m-CPBA (77 mg, 0.45 mmol, 1.99 equiv) and 6,6-dimethyl-N-(1-((1-methylpiperidin-4-yl)(phenyl)methyl)-1H-pyrazol-4-yl)-4,5,6,7-tetrahydro-1H-indazole-3-carboxamide (100 mg, 0.22 mmol, 1.00 equiv; Example 43, racemic) in dichloromethane (10 mL)/methanol (2 mL) was stirred for 1 h at room temperature. The reaction was then quenched by 5 mL of saturated sodium bicarbonate, extracted with ethyl acetate, dried over anhydrous sodium sulfate, and concentrated under vacuum. Purification by SFC using a chiral stationary phase provided the desired product as single enantiomers.

SFC conditions: ChiralCel OD-H (4.6×100 mm, 5 μm particle size) at 20% methanol+0.1% DEA; 5 mL/min, 100 bars, 40° C.

140a: ¹H NMR (300 MHz, CDCl₃) δ 8.16 (s, 1H), 7.71 (s, 1H), 7.56-7.53 (m, 2H), 7.40-7.28 (m, 3H), 5.04 (d, J=10.8 Hz, 1H), 3.42-3.19 (m, 4H), 3.17 (s, 3H), 2.78 (t, J=6.3 Hz, 2H), 2.72-2.64 (m, 1H), 2.43 (s, 1H), 2.11-1.92 (m, 2H), 1.03 (s, 6H); MS: m/z=463 (M+H); SFC retention time: 2.9 min.

140b: ¹H NMR (300 MHz, CDCl₃) δ 8.14 (s, 1H), 7.71 (s, 1H), 7.56-7.53 (m, 2H), 7.40-7.28 (m, 3H), 5.04 (d, J=10.8 Hz, 1H), 3.42-3.20 (m, 4H), 3.17 (s, 3H), 2.80-2.76 (t, J=6.3 Hz, 2H,), 2.72-2.64 (m, 1H), 2.43 (s, 1H), 2.11-1.92 (m, 2H), 1.03 (s, 6H); MS: m/z=463 (M+H); SFC retention time: 3.7 min.

### Examples 141

### N-(1-((1,1-dioxidotetrahydro-2H-thiopyran-4-yl)(phenyl)methyl)-1H-pyrazol-4-yl)-5,5-difluoro-5a-methyl-1,4,4a,5,5a,6-hexahydrocyclopropa[f]indazole-3-carboxamide

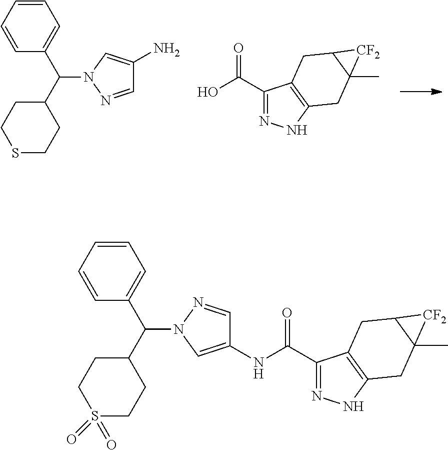 [SMILES: Nc1cnn(C(c2ccccc2)C2CCSCC2)c1 ; CC12Cc3[nH]nc(C(=O)O)c3CC1C2(F)F ; CC12Cc3[nH]nc(C(=O)Nc4cnn(C(c5ccccc5)C5CCS(=O)(=O)CC5)c4)c3CC1C2(F)F]

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with 1-(phenyl(tetrahydro-2H-thiopyran-4-yl)methyl)-1H-pyrazol-4-amine (Example A83) and 6,6-dimethyl-1-(2-trimethylsilylethoxymethyl)-5,7-dihydro-4H-indazole-3-carboxylic acid (Example C6) with 5,5-difluoro-5a-methyl-1,4,4a,5,5a,6-hexahydrocyclopropa[f]indazole-3-carboxylic acid (Example C43). In this case, deprotection and chiral separation are not necessary.

141: ¹H NMR (300 MHz, DMSO-d₆) δ 12.94 (s, 1H), 10.16 (s, 1H), 8.17 (s, 1H), 7.68 (s, 1H), 7.54 (d, J=4.5 Hz, 2H), 7.39-7.27 (m, 3H), 5.30 (d, J=4.5 Hz, 1H), 3.16-2.96 (m, 7H), 2.98 (d, J=6.0 Hz, 2H), 1.79-1.60 (m, 5H), 1.35 (s, 3H); MS: m/z=516 (M+H).

### Examples 142a and 142b

### 5,5-difluoro-5a-methyl-N-(1-((1-oxidotetrahydro-2H-thiopyran-4-yl)(phenyl)methyl)-1H-pyrazol-4-yl)-1,4,4a,5,5a,6-hexahydrocyclopropa[f]indazole-3-carboxamide

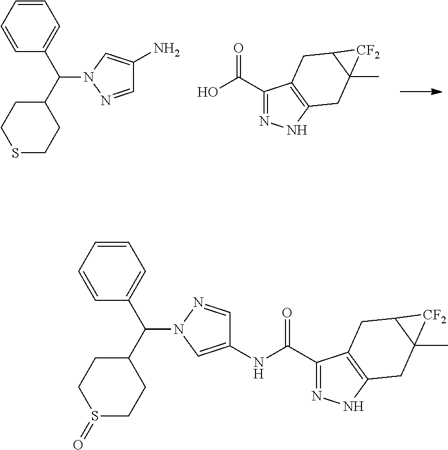 [SMILES: Nc1cnn(C(c2ccccc2)C2CCSCC2)c1 ; CC12Cc3[nH]nc(C(=O)O)c3CC1C2(F)F ; CC12Cc3[nH]nc(C(=O)Nc4cnn(C(c5ccccc5)C5CCS(=O)CC5)c4)c3CC1C2(F)F]

Prepared in an analogous manner to N-(1-((1,1-dioxidotetrahydro-2H-thiopyran-4-yl)(phenyl)methyl)-1H-pyrazol-4-yl)-5,5-difluoro-5a-methyl-1,4,4a,5,5a,6-hexahydrocyclopropa[f]indazole-3-carboxamide (Example 141), except only a single equivalent of mCPBA was used in the oxidation step. The diasetereomers were separated by SFC using a chiral stationary phase.

SFC conditions: ChiralCel OD-H (4.6×100 mm, 3 μm particle size) at 50% methanol+0.1% DEA; 5 mL/min, 100 bars, 40° C.

142a: ¹H NMR (300 MHz, DMSO-d₆) δ 12.94 (s, 1H), 10.15 (s, 1H), 8.17 (s, 1H), 7.67 (s, 1H), 7.54 (d, J=6.9 Hz, 2H), 7.38-7.28 (m, 3H), 5.16 (d, J=10.5 Hz, 1H), 3.11-3.00 (m, 3H), 2.84-2.73 (m, 3H), 2.66-2.49 (m, 3H), 1.95-1.75 (m, 3H), 1.35 (s, 3H), 1.31-1.21 (m, 2H); MS: m/z=500 (M+H); SFC retention time: 2.2 min.

142b: ¹H NMR (300 MHz, DMSO-d₆) δ 12.94 (s, 1H), 10.16 (s, 1H), 8.16 (s, 1H), 7.66 (s, 1H), 7.54 (d, J=6.9 Hz, 2H), 7.38-7.29 (m, 3H), 5.29 (d, J=10.8 Hz, 1H), 3.32-3.00 (m, 5H), 2.84-2.61 (m, 2H), 2.58-2.50 (m, 2H), 1.79-1.74 (m, 3H), 1.35-1.22 (m, 5H); MS: m/z=500 (M+H); SFC retention time: 1.2 min.

### Examples 143a-d

### N-(1-((1,1-dioxidotetrahydro-2H-thiopyran-2-yl)(phenyl)methyl)-1H-pyrazol-4-yl)-5a-methyl-1,4,4a,5,5a,6-hexahydrocyclopropa[f]indazole-3-carboxamide

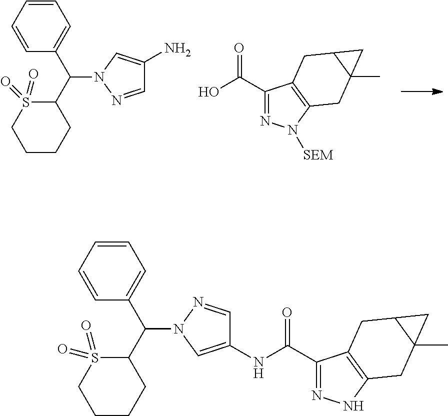 [SMILES: Nc1cnn(C(c2ccccc2)C2CCCCS2(=O)=O)c1 ; CC12Cc3c(c(C(=O)O)nn3COCC[Si](C)(C)C)CC1C2 ; CC12Cc3[nH]nc(C(=O)Nc4cnn(C(c5ccccc5)C5CCCCS5(=O)=O)c4)c3CC1C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5a-methyl-1-((2-(trimethylsilyl)ethoxy)methyl)-1,4,4a,5,5a,6-hexahydrocyclopropa[f]indazole-3-carboxylic acid (Example C31a) and 3-((4-amino-1H-pyrazol-1-yl)methyl)benzonitrile (Example A1) with 2-((4-amino-1H-pyrazol-1-yl)(phenyl)methyl)tetrahydro-2H-thiopyran 1,1-dioxide (Example A76).

SFC conditions: ChiralPak IA (4.6×100 mm, 3 μm particle size) at 50% IPA+0.1% DEA; 5 mL/min, 100 bars, 40° C.

143a: ¹H NMR (300 MHz, DMSO-d₆) δ 12.86 (s, 1H), 10.10 (s, 1H), 8.22 (s, 1H), 7.66 (s, 1H), 7.60-7.57 (m, 2H), 7.33-7.28 (m, 2H), 5.71 (d, J=9.9 Hz, 1H), 4.37-4.36 (m, 1H), 3.31-2.81 (m, 5H), 2.70-2.64 (m, 1H), 2.00-1.92 (m, 1H), 1.71-1.40 (m, 5H), 1.21 (s, 3H), 1.04-1.01 (m, 1H), 0.36-0.32 (m, 1H), 0.10-0.07 (m, 1H); MS: m/z=480 (M+H); SFC retention time: 1.7 min.

143b: ¹H NMR (300 MHz, DMSO-d₆) δ 12.86 (s, 1H), 10.10 (s, 1H), 8.22 (s, 1H), 7.66 (s, 1H), 7.60-7.58 (m, 2H), 7.34-7.28 (m, 3H), 5.70 (d, J=10.2 Hz, 1H), 4.37-4.36 (m, 1H), 3.31-2.81 (m, 5H), 2.70-2.65 (m, 1H), 1.92-1.40 (m, 6H), 1.21 (s, 3H), 1.04-1.01 (m, 1H), 0.36-0.33 (m, 1H), 0.10-0.07 (m, 1H); MS: m/z=480 (M+H); SFC retention time: 2.0 min.

143c: ¹H NMR (300 MHz, DMSO-d₆) δ 12.84 (s, 1H), 10.07 (s, 1H), 8.17 (s, 1H), 7.63 (s, 1H), 7.52 (m, 2H), 7.50-7.29 (m, 3H), 5.81 (d, J=9.3 Hz, 1H), 4.49-4.46 (m, 1H), 3.28-3.15 (m, 3H), 2.99-2.70 (m, 2H), 2.65-2.49 (m, 1H), 1.90-1.21 (m, 9H), 1.03-1.01 (m, 1H), 0.36-0.31 (m, 1H), 0.10-0.01 (m, 1H); MS: m/z=480 (M+H); SFC retention time: 2.5 min.

143d: ¹H NMR (300 MHz, DMSO-d₆) δ 12.84 (s, 1H), 10.07 (s, 1H), 8.18 (s, 1H), 7.63 (s, 1H), 7.52-7.49 (m, 2H), 7.38-7.29 (m, 3H), 5.82 (d, J=9.3 Hz, 1H), 4.52-4.46 (m, 1H), 3.31-3.15 (m, 3H), 2.99-2.49 (m, 3H), 2.00-1.21 (m, 9H), 1.03-1.01 (m, 1H), 0.36-0.31 (m, 1H), 0.10-0.01 (m, 1H); MS: m/z=480 (M+H); SFC retention time: 3.3 min.

### Examples 144a-d

### N-(1-((1,1-dioxidotetrahydro-2H-thiopyran-2-yl)(pyridin-3-yl)methyl)-1H-pyrazol-4-yl)-6,6-dimethyl-4,5,6,7-tetrahydro-1H-indazole-3-carboxamide

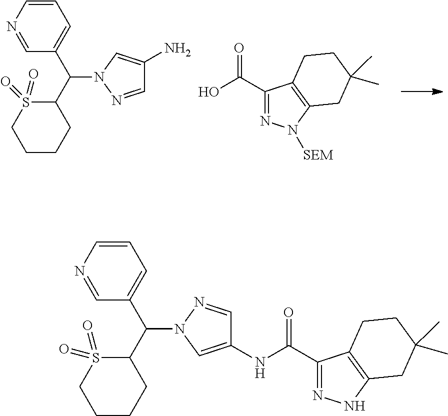 [SMILES: Nc1cnn(C(c2cccnc2)C2CCCCS2(=O)=O)c1 ; CC1(C)CCc2c(C(=O)O)nn(COCC[Si](C)(C)C)c2C1 ; CC1(C)CCc2c(C(=O)Nc3cnn(C(c4cccnc4)C4CCCCS4(=O)=O)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 2-((4-amino-1H-pyrazol-1-yl)(phenyl)methyl)tetrahydro-2H-thiopyran 1,1-dioxide (Example A76).

SFC conditions: ChiralPak IA (4.6×100 mm, 3 μm particle size) at 50% (2:1 MeOH:DCM+0.2% DEA); 5 mL/min, 100 bars, 40° C.

144a: ¹H NMR (300 MHz, CD₃OD) δ 8.79 (1H, s), 8.47-8.45 (m, 1H), 8.21-8.15 (m, 2H), 7.74 (s, 1H), 7.43-7.39 (m, 1H), 5.75 (d, J=10.5 Hz, 1H), 4.44-4.37 (m, 1H), 3.17-3.03 (m, 2H), 2.77 (t, J=6.3 Hz, 2H), 2.43 (s, 2H), 2.05-1.95 (m, 2H), 1.86-1.73 (m, 2H), 1.63-1.54 (m, 4H), 1.02 (s, 6H); MS: m/z=483 (M+H); SFC retention time: 1.4 min.

144b: ¹H NMR (300 MHz, CD₃OD) δ 8.79 (s, 1H), 8.47-8.45 (m, 1H), 8.21-8.15 (m, 2H), 7.74 (s, 1H), 7.43-7.39 (m, 1H), 5.75 (d, J=10.5 Hz, 1H), 4.44-4.39 (m, 1H), 3.17-3.03 (m, 2H), 2.77 (t, J=6.3 Hz, 2H,), 2.42 (s, 2H), 2.05-1.95 (m, 2H), 1.85-1.73 (m, 2H), 1.62-1.54 (m, 4H), 1.02 (s, 6H); MS: m/z=483 (M+H); SFC retention time: 2.2 min.

144c: ¹H NMR (300 MHz, CD₃OD) δ 8.71 (s, 1H), 8.52-8.50 (m, 1H), 8.17-8.05 (m, 2H), 7.73 (s, 1H), 7.47-7.42 (m, 1H), 5.99 (d, J=9.0 Hz, 1H), 4.49-4.43 (m, 1H), 3.18-3.11 (m, 2H), 2.76 (t, J=8.4 Hz, 2H), 2.42 (s, 2H), 2.09-1.93 (m, 2H), 1.82-1.78 (m, 2H), 1.65-1.57 (m, 4H), 1.01 (s, 6H); MS: m/z=483 (M+H); SFC retention time: 3.5 min.

144d: ¹H NMR (300 MHz, CD₃OD) δ 8.71 (s, 1H), 8.52-8.50 (m, 1H), 8.17-8.05 (m, 2H), 7.73 (s, 1H), 7.47-7.43 (m, 1H), 5.98 (d, J=9.0 Hz, 1H), 4.49-4.43 (m, 1H), 3.18-3.08 (m, 2H), 2.77 (t, J=8.4 Hz, 2H), 2.42 (s, 2H), 2.09-1.93 (m, 2H), 1.82-1.79 (m, 2H), 1.66-1.62 (m, 4H), 1.01 (s, 6H); MS: m/z=483 (M+H); SFC retention time: 5.7 min.

### Examples 145a-d

### N-(1-((1,1-dioxidotetrahydro-2H-thiopyran-2-yl)(phenyl)methyl)-1H-pyrazol-4-yl)-5,5-difluoro-5a-methyl-1,4,4a,5,5a,6-hexahydrocyclopropa[f]indazole-3-carboxamide

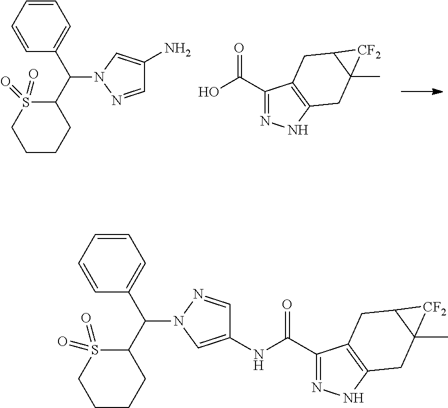 [SMILES: Nc1cnn(C(c2ccccc2)C2CCCCS2(=O)=O)c1 ; CC12Cc3[nH]nc(C(=O)O)c3CC1C2(F)F ; CC12Cc3[nH]nc(C(=O)Nc4cnn(C(c5ccccc5)C5CCCCS5(=O)=O)c4)c3CC1C2(F)F]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5,5-difluoro-5a-methyl-1,4,4a,5,5a,6-hexahydrocyclopropa[f]indazole-3-carboxylic acid (Example C43) and 3-((4-amino-1H-pyrazol-1-yl)methyl)benzonitrile (Example A1) with 2-((4-amino-1H-pyrazol-1-yl)(pyridin-3-yl)methyl)tetrahydro-2H-thiopyran 1,1-dioxide (Example A80). The final deprotection step is not necessary in this case.

SFC conditions: ChiralCel OJ-3 (4.6×100 mm, 3 μm particle size) at 5-40% (MeOH+0.1% DEA); 5 mL/min, 100 bars, 40° C.

145a: ¹H NMR (300 MHz, CDCl₃) δ 8.94 (s, 1H), 7.91 (s, 1H), 7.67 (s, 1H), 7.44 (d, J=3.0 Hz, 2H), 7.34-7.30 (m, 3H), 5.68 (d, J=4.5 Hz, 1H), 4.42-4.35 (m, 1H), 3.50 (s, 2H), 3.30-2.94 (m, 6H), 2.69 (d, J=9.0 Hz, 2H), 2.02 (s, 2H), 1.94-1.57 (m, 4H), 1.51-1.49 (m, 1H), 1.39 (s, 3H); MS: m/z=516 (M+H); SFC retention time: 3.4 min.

145b: ¹H NMR (300 MHz, CD₃OD) δ 8.15 (s, 1H), 7.69 (s, 1H), 7.53 (d, J=1.5 Hz, 1H), 7.51-7.33 (m, 3H), 5.79 (d, J=4.5 Hz, 1H), 4.86-4.41 (m, 1H), 3.33-3.31 (m, 1H), 3.22-3.04 (m, 5H), 2.82-2.76 (m, 1H), 2.09-1.94 (m, 2H), 1.39 (s, 3H), 1.30 (s, 1H); MS: m/z=516 (M+H); SFC retention time: 3.6 min.

145c: ¹H NMR (300 MHz, DMSO-d₆) δ 13.00 (s, 1H), 10.20 (s, 1H), 8.24 (s, 1H), 7.67 (s, 1H), 7.61 (d, J=1.5 Hz, 2H), 7.58-7.27 (m, 3H), 5.71 (d, J=4.5 Hz, 1H), 4.37 (t, J=10.5 Hz, 1H), 4.10-4.08 (m, 4H), 3.22-3.13 (m, 14H), 3.08-3.00 (m, 4H), 2.84-2.77 (m, 1H), 1.97-1.93 (br, 1H), 1.80-1.39 (m, 6H), 1.35 (s, 3H); MS: m/z=516 (M+H); SFC retention time: 4.2 min.

145d: ¹H NMR (300 MHz, DMSO-d₆) δ 13.00 (s, 1H), 10.20 (s, 1H), 8.23 (s, 1H), 7.67 (s, 1H), 7.60 (d, J=1.5 Hz, 2H), 7.58-7.24 (m, 3H), 5.71 (d, J=3.0 Hz, 1H), 4.46 (t, J=10.5 Hz, 1H), 4.10-4.08 (m, 4H), 3.23-3.00 (m, 5H), 2.84-2.73 (m, 1H), 1.98-1.92 (br, 1H), 1.85-1.40 (m, 6H), 1.35 (s, 3H); MS: m/z=516 (M+H); SFC retention time: 5.8 min.

### Examples 146a-d

### 5a-methyl-N-(1-((1-oxidotetrahydro-2H-thiopyran-4-yl)(pyridin-3-yl)methyl)-1H-pyrazol-4-yl)-1,4,4a,5,5a,6-hexahydrocyclopropa[f]indazole-3-carboxamide

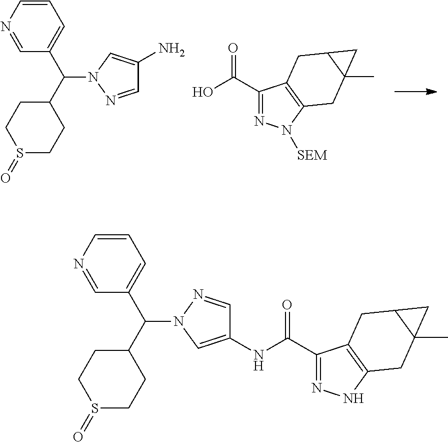 [SMILES: Nc1cnn(C(c2cccnc2)C2CCS(=O)CC2)c1 ; CC12Cc3c(c(C(=O)O)nn3COCC[Si](C)(C)C)CC1C2 ; CC12Cc3[nH]nc(C(=O)Nc4cnn(C(c5cccnc5)C5CCS(=O)CC5)c4)c3CC1C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5a-methyl-1-((2-(trimethylsilyl)ethoxy)methyl)-1,4,4a,5,5a,6-hexahydrocyclopropa[f]indazole-3-carboxylic acid (Example C31a) and 3-((4-amino-1H-pyrazol-1-yl)methyl)benzonitrile (Example A1) with 4-((4-amino-1H-pyrazol-1-yl)(pyridin-3-yl)methyl)tetrahydro-2H-thiopyran 1-oxide (Example A81).

SFC conditions: ChiralPak IC (4.6×100 mm, 3 μm particle size) at 50% (MeOH+0.1% DEA); 5 mL/min, 100 bars, 40° C.

146a: ¹H NMR (300 MHz, DMSO-d₆) δ 12.95 (s, 1H), 10.11 (s, 1H), 8.75-8.74 (m, 1H), 8.52-8.50 (m, 1H), 8.20 (s, 1H), 8.00-7.96 (m, 1H), 7.70 (s, 1H), 7.42-7.38 (m, 1H), 5.32-5.28 (m, 1H), 3.24-3.18 (m, 1H), 2.99-2.94 (m, 1H), 2.88-2.79 (m, 3H), 2.70-2.51 (m, 4H), 1.96-1.82 (m, 2H), 1.21-1.15 (m, 5H), 1.06-0.99 (m, 1H), 0.36-0.32 (m, 1H), 0.11-0.08 (m, 1H); MS: m/z=465 (M+H); SFC retention time: 1.9 min.

146b: ¹H NMR (300 MHz, DMSO-d₆) δ 12.95 (s, 1H), 10.14 (s, 1H), 8.74-8.73 (m, 1H), 8.53-8.50 (m, 1H), 8.20 (s, 1H), 8.00-7.98 (m, 1H), 7.69 (s, 1H), 7.43-7.38 (m, 1H), 5.43-5.39 (m, 1H), 3.33-3.13 (m, 1H), 3.10-2.94 (m, 1H), 2.89-2.76 (m, 3H), 2.71-2.51 (m, 4H), 1.80-1.69 (m, 2H), 1.30-1.21 (m, 5H), 1.04-0.99 (m, 1H), 0.36-0.32 (m, 1H), 0.11-0.07 (m, 1H); MS: m/z=465 (M+H); SFC retention time: 2.6 min.

146c: ¹H NMR: (300 MHz, DMSO-d₆) δ 12.95 (s, 1H), 10.11 (s, 1H), 8.73-8.72 (m, 1H), 8.52-8.50 (m, 1H), 8.19 (s, 1H), 8.00-7.97 (m, 1H), 7.68 (s, 1H), 7.42-7.38 (m, 1H), 5.43-5.39 (m, 1H), 3.23-3.16 (m, 3H), 2.99-2.81 (m, 4H), 2.70-2.61 (m, 2H), 1.80-1.60 (m, 2H), 1.71-1.21 (m, 5H), 1.07-1.01 (m, 1H), 0.36-0.32 (m, 1H), 0.11-0.01 (m, 1H); MS: m/z=465 (M+H); SFC retention time: 4.5 min.

146d: ¹H NMR (300 MHz, DMSO-d₆,) δ 12.95 (s, 1H), 10.11 (s, 1H), 8.75-8.74 (m, 1H), 8.52-8.50 (m, 1H), 8.20 (s, 1H), 8.00-7.99 (m, 1H), 7.70 (s, 1H), 7.42-7.38 (m, 1H), 5.32-5.28 (m, 1H), 3.33-3.17 (m, 1H), 2.99-2.89 (m, 1H), 2.89-2.70 (m, 3H), 2.65-2.51 (m, 4H), 1.92-1.82 (m, 2H), 1.29-1.23 (m, 5H), 1.04-1.01 (m, 1H), 0.36-0.32 (m, 1H), 0.11-0.07 (m, 1H); MS: m/z=465 (M+H); SFC retention time: 6.1 min.

### Examples 147a-d

### 6,6-dimethyl-N-(1-((1-oxidotetrahydro-2H-thiopyran-4-yl)(pyridin-3-yl)methyl)-1H-pyrazol-4-yl)-4,5,6,7-tetrahydro-1H-indazole-3-carboxamide

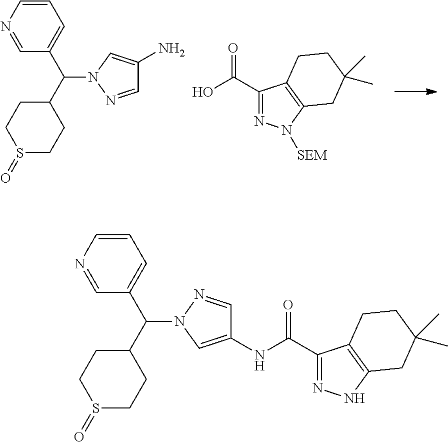 [SMILES: Nc1cnn(C(c2cccnc2)C2CCS(=O)CC2)c1 ; CC1(C)CCc2c(C(=O)O)nn(COCC[Si](C)(C)C)c2C1 ; CC1(C)CCc2c(C(=O)Nc3cnn(C(c4cccnc4)C4CCS(=O)CC4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6,6-dimethyl-1-((2-(trimethylsilyl)ethoxy)methyl)-4,5,6,7-tetrahydro-1H-indazole-3-carboxylic acid (Example C6) and 3-((4-amino-1H-pyrazol-1-yl)methyl)benzonitrile (Example A1) with 4-((4-amino-1H-pyrazol-1-yl)(pyridin-3-yl)methyl)tetrahydro-2H-thiopyran 1-oxide (Example A81).

SFC conditions: ChiralPak IA (4.6×100 mm, 3 μm particle size) at 50% (2:1:1 MeOH:DCM:ACN+0.2% DEA); 5 mL/min, 100 bars, 40° C.

147a: ¹H NMR (300 MHz, DMSO-d₆) δ 12.80 (s, 1H), 10.11 (s, 1H), 8.75 (d, J=1.8 Hz, 1H), 8.51 (q, J=2.1 Hz, 1H), 8.21 (s, 1H), 8.00-7.99 (m, 1H), 7.71 (s, 1H), 7.40 (q, J=4.3 Hz, 1H), 5.30 (d, J=10.8 Hz, 1H), 2.86-2.50 (m, 7H), 2.38 (s, 2H), 1.96-1.78 (m, 2H), 1.47 (t, J=6.3 Hz, 2H), 1.19-1.15 (m, 2H), 0.98 (s, 6H); MS: m/z=467 (M+H); SFC retention time: 1.8 min.

147b: ¹H NMR (300 MHz, DMSO-d₆) δ 12.80 (s, 1H), 10.10 (s, 1H), 8.75 (d, J=1.8 Hz, 1H), 8.52 (q, J=2.1 Hz, 1H), 8.21 (s, 1H), 8.00-7.96 (m, 1H), 7.71 (s, 1H), 7.40 (q, J=4.3 Hz, 1H), 5.30 (d, J=10.8 Hz, 1H), 2.86-2.49 (m, 7H), 2.38 (s, 2H), 1.92-1.78 (m, 2H), 1.47 (t, J=6.3 Hz, 2H), 1.19-1.15 (m, 2H), 0.98 (s, 6H); MS: m/z=467 (M+H); SFC retention time: 2.1 min.

147c: ¹H NMR (300 MHz, DMSO-d₆) δ 12.81 (br, 1H), 10.15 (br, 1H), 8.73 (d, J=1.8 Hz, 1H), 8.51 (q, J=2.2 Hz, 1H), 8.21 (s, 1H), 8.01-7.97 (m, 1H), 7.69 (s, 1H), 7.40 (q, J=4.1 Hz, 1H), 5.41 (d, J=11.1 Hz, 1H), 3.22-3.09 (m, 2H), 2.79-2.51 (m, 5H), 2.50 (s, 2H), 1.69 (m, 2H), 1.47 (t, J=6.2 Hz, 2H), 1.33-1.31 (m, 2H), 0.97 (s, 6H); MS: m/z=467 (M+H); SFC retention time: 5.1 min.

147d: ¹H NMR (300 MHz, DMSO-d₆) δ 12.80 (s, 1H), 10.11 (s, 1H), 8.73 (d, J=1.5 Hz, 1H), 8.51 (q, J=2.1 Hz, 1H), 8.21 (s, 1H), 8.01-7.97 (m, 1H), 7.70 (s, 1H), 7.40 (q, J=4.2 Hz, 1H), 5.41 (d, J=11.1 Hz, 1H), 3.22-3.10 (m, 2H), 2.79-2.51 (m, 5H), 2.50 (s, 2H), 1.69 (m, 2H), 1.47 (t, J=6.2 Hz, 2H), 1.35-1.30 (m, 2H), 0.97 (s, 6H); MS: m/z=467 (M+H); SFC retention time: 6.1 min.

### Examples 148a and 148b

### N-(1-benzyl-1H-pyrazol-4-yl)-5′-oxo-1,4,5,7-tetrahydrospiro[indazole-6,2′-pyrrolidine]-3-carboxamide

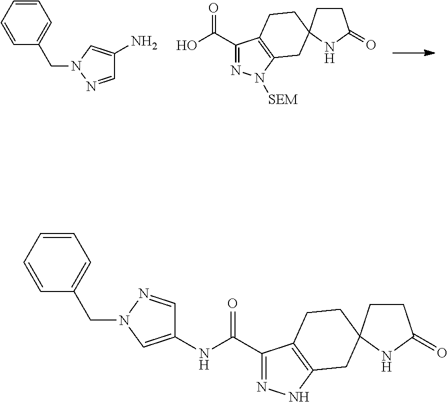 [SMILES: Nc1cnn(Cc2ccccc2)c1 ; C[Si](C)(C)CCOCn1nc(C(=O)O)c2c1CC1(CCC(=O)N1)CC2 ; O=C1CCC2(CCc3c(C(=O)Nc4cnn(Cc5ccccc5)c4)n[nH]c3C2)N1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5′-oxo-1-((2-(trimethylsilyl)ethoxy)methyl)-1,4,5,7-tetrahydrospiro[indazole-6,2′-pyrrolidine]-3-carboxylic acid (Example C38) and 3-((4-amino-1H-pyrazol-1-yl)methyl)benzonitrile (Example A1) with 1-benzyl-1H-pyrazol-4-amine (Example A2).

SFC Conditions: Lux Cellulose 3 (4.6×50 mm, 5 μm particle size) at 25% methanol w/0.1% NH₄OH; 5 mL/min, 120 bars, 40° C.

148a: ¹H NMR (400 MHz, DMSO-d₆) δ 12.87 (s, 1H), 10.11 (s, 1H), 8.08 (s, 1H), 7.85 (s, 1H), 7.64 (s, 1H), 7.40-7.17 (m, 5H), 5.27 (s, 2H), 2.89-2.62 (m, 4H), 2.35-2.15 (m, 2H), 1.98-1.63 (m, 4H); MS: m/z=391 (M+H); SFC retention time: 0.7 min.

148b: ¹H NMR (400 MHz, DMSO-d₆) δ 12.87 (s, 1H), 10.12 (s, 1H), 8.08 (s, 1H), 7.85 (s, 1H), 7.65 (s, 1H), 7.38-7.20 (m, 5H), 5.27 (s, 2H), 3.06-2.60 (m, 4H), 2.35-2.15 (m, 2H), 1.95-1.63 (m, 4H); MS: m/z=391 (M+H); SFC retention time: 1.0 min.

### Examples 149a and 149b

### N-(1-benzyl-1H-pyrazol-4-yl)-6-methyl-6-morpholino-4,5,6,7-tetrahydro-1H-indazole-3-carboxamide

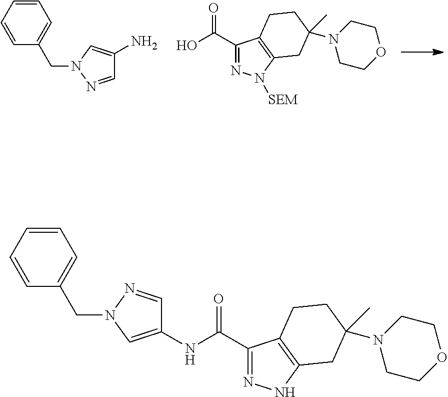 [SMILES: Nc1cnn(Cc2ccccc2)c1 ; CC1(N2CCOCC2)CCc2c(C(=O)O)nn(COCC[Si](C)(C)C)c2C1 ; CC1(N2CCOCC2)CCc2c(C(=O)Nc3cnn(Cc4ccccc4)c3)n[nH]c2C1]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 6-methyl-6-morpholino-1-((2-(trimethylsilyl)ethoxy)methyl)-4,5,6,7-tetrahydro-1H-indazole-3-carboxylic acid (Example C44) and 3-((4-amino-1H-pyrazol-1-yl)methyl)benzonitrile (Example A1) with 1-benzyl-1H-pyrazol-4-amine (Example A2).

SFC Conditions: Lux Cellulose 1 (4.6×50 mm, 5 μm particle size) at 40% methanol w/0.1% NH₄OH; 5 mL/min, 120 bars, 40° C.

149a: ¹H NMR (400 MHz, DMSO-d₆) δ 12.77 (s, 1H), 10.07 (s, 1H), 8.07 (d, J=0.5 Hz, 1H), 7.64 (d, J=0.5 Hz, 1H), 7.38-7.20 (m, 5H), 5.27 (s, 2H), 3.60-3.45 (m, 4H), 2.82-2.70 (m, 2H), 2.62-2.41 (m, 6H), 1.78-1.60 (m, 2H), 0.96 (s, 3H); MS: m/z=421 (M+H); SFC retention time: 0.56 min.

149b: ¹H NMR (400 MHz, DMSO-d₆) δ 12.77 (s, 1H), 10.07 (s, 1H), 8.07 (s, 1H), 7.64 (s, 1H), 7.37-7.20 (m, 5H), 5.27 (s, 2H), 3.59-3.46 (m, 4H), 2.81-2.71 (m, 2H), 2.63-2.41 (m, 6H), 1.77-1.62 (m, 2H), 0.96 (s, 3H); MS: m/z=421 (M+H); SFC retention time: 0.68 min.

### Examples 150a and 150b

### N-(1-benzyl-1H-pyrazol-4-yl)-4a-methyl-3b,4,4a,5-tetrahydro-1H-cyclopropa[3,4]cyclopenta[1,2-c]pyrazole-3-carboxamide

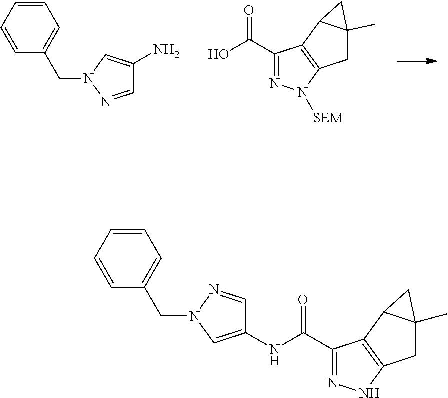 [SMILES: Nc1cnn(Cc2ccccc2)c1 ; CC12Cc3c(c(C(=O)O)nn3COCC[Si](C)(C)C)C1C2 ; CC12Cc3[nH]nc(C(=O)Nc4cnn(Cc5ccccc5)c4)c3C1C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 4a-methyl-1-((2-(trimethylsilyl)ethoxy)methyl)-3b,4,4a,5-tetrahydro-1H-cyclopropa[3,4]cyclopenta[1,2-c]pyrazole-3-carboxylic acid (Example C41) and 3-((4-amino-1H-pyrazol-1-yl)methyl)benzonitrile (Example A1) with 1-benzyl-1H-pyrazol-4-amine (Example A2).

SFC Conditions: ChiralPak ID (4.6×50 mm, 5 μm particle size) at 45% methanol w/0.1% NH₄OH; 5 mL/min, 120 bars, 40° C.

150a: ¹H NMR (400 MHz, DMSO-d₆) δ 12.89-12.57 (m, 1H), 10.12-9.67 (m, 1H), 8.14-8.00 (m, 1H), 7.70-7.56 (m, 1H), 7.40-7.16 (m, 5H), 5.34-5.25 (m, 2H), 2.91-2.59 (m, 2H), 2.31-1.87 (m, 1H), 1.46-1.33 (m, 3H), 1.09-0.91 (m, 1H), 0.36-0.25 (m, 1H); MS: m/z=334 (M+H); SFC retention time: 0.6 min.

150b: ¹H NMR (400 MHz, DMSO-d₆) δ 12.85-12.62 (m, 1H), 10.09-9.71 (m, 1H), 8.10-8.03 (m, 1H), 7.68-7.59 (m, 1H), 7.39-7.18 (m, 5H), 5.34-5.24 (m, 2H), 2.88-2.62 (m, 2H), 2.30-1.88 (m, 1H), 1.46-1.33 (m, 3H), 1.09-0.90 (m, 1H), 0.35-0.24 (m, 1H); MS: m/z=334 (M+H); SFC retention time: 1.4 min.

### Examples 151a and 151b

### N-(1-benzyl-1H-pyrazol-4-yl)-4a-methyl-4,4a,5,5a-tetrahydro-H-cyclopropa[4,5]cyclopenta[1,2-c]pyrazole-3-carboxamide

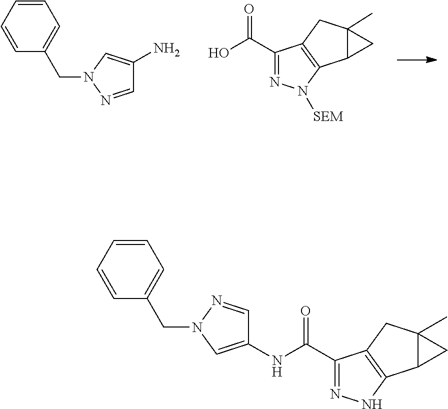 [SMILES: Nc1cnn(Cc2ccccc2)c1 ; CC12Cc3c(C(=O)O)nn(COCC[Si](C)(C)C)c3C1C2 ; CC12Cc3c(C(=O)Nc4cnn(Cc5ccccc5)c4)n[nH]c3C1C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 4a-methyl-1-((2-(trimethylsilyl)ethoxy)methyl)-4,4a,5,5a-tetrahydro-1H-cyclopropa[4,5]cyclopenta[1,2-c]pyrazole-3-carboxylic acid (Example C41) and 3-((4-amino-1H-pyrazol-1-yl)methyl)benzonitrile (Example A1) with 1-benzyl-1H-pyrazol-4-amine (Example A2).

SFC Conditions: Lux Cellulose 1 (4.6×50 mm, 5 μm particle size) at 25% methanol w/0.1% NH₄OH; 5 mL/min, 120 bars, 40° C.

151a: ¹H NMR (400 MHz, DMSO-d₆) δ 12.95-12.48 (m, 1H), 10.13-9.47 (m, 1H), 8.04 (s, 1H), 7.62 (s, 1H), 7.38-7.19 (m, 5H), 5.27 (s, 2H), 2.99-2.55 (m, 2H), 2.01-1.84 (m, 1H), 1.39 (s, 3H), 1.07-0.99 (m, 1H), 0.46-0.32 (m, 1H); MS: m/z=334 (M+H); SFC retention time: 0.7 min.

151b: ¹H NMR (400 MHz, DMSO-d₆) δ 12.99-12.45 (m, 1H), 10.13-9.44 (m, 1H), 8.04 (s, 1H), 7.62 (s, 1H), 7.37-7.20 (m, 5H), 5.27 (s, 2H), 3.01-2.56 (m, 2H), 2.02-1.84 (m, 1H), 1.39 (s, 3H), 1.09-0.99 (m, 1H), 0.49-0.29 (m, 1H); MS: m/z=334 (M+H); SFC retention time: 0.9 min.

### Examples 152a-d

### N-(1-((1,1-dioxidotetrahydro-2H-thiopyran-4-yl)(phenyl)methyl)-1H-pyrazol-4-yl)-4a-methyl-4,4a,5,5a-tetrahydro-1H-cyclopropa[4,5]cyclopenta[1,2-c]pyrazole-3-carboxamide

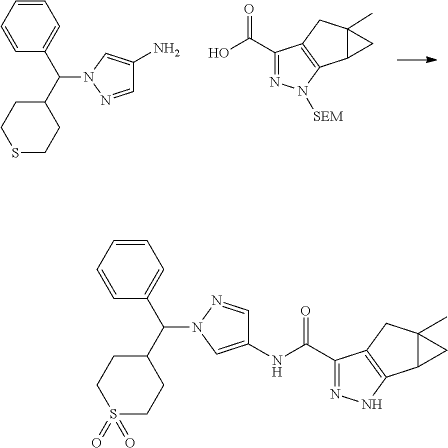 [SMILES: Nc1cnn(C(c2ccccc2)C2CCSCC2)c1 ; CC12Cc3c(C(=O)O)nn(COCC[Si](C)(C)C)c3C1C2 ; CC12Cc3c(C(=O)Nc4cnn(C(c5ccccc5)C5CCS(=O)(=O)CC5)c4)n[nH]c3C1C2]

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with 1-(phenyl(tetrahydro-2H-thiopyran-4-yl)methyl)-1H-pyrazol-4-amine (Example A55) and 6,6-dimethyl-1-((2-(trimethylsilyl)ethoxy)methyl)-4,5,6,7-tetrahydro-1H-indazole-3-carboxylic acid (Example C6) with 4a-methyl-1-((2-(trimethylsilyl)ethoxy)methyl)-4,4a,5,5a-tetrahydro-1H-cyclopropa[4,5]cyclopenta[1,2-c]pyrazole-3-carboxylic acid (Example C40). In this case, the pyrazole carboxylate (Example C6) was first separated into its constituent enantiomers using SFC with a chiral stationary phase (Whelk O1; 15% MeOH+0.1% NH₄OH), then each enantiomer was carried into the amide bond formation separately.

SFC Conditions to separate final compounds:

Diastereomeric pair 1 (152a and b): Whelk O1 (4.6×50 mm, 5 μm particle size) at 50% methanol w/0.1% NH₄OH; 5 mL/min, 120 bars, 40° C.

Diastereomeric pair 2 (152c and d): Lux Cellulose 1 (4.6×50 mm, 5 μm particle size) at 50% methanol w/0.10% NH₄OH; 5 mL/min, 120 bars, 40° C.

152a: ¹H NMR (400 MHz, DMSO-d₆) δ 12.96-12.49 (m, 1H), 10.09-9.53 (m, 1H), 8.12 (s, 1H), 7.70-7.50 (m, 3H), 7.40-7.25 (m, 3H), 5.42-5.23 (m, 1H), 3.19-2.55 (m, 7H), 2.01-1.85 (m, 1H), 1.76-1.50 (m, 4H), 1.39 (s, 3H), 1.10-0.97 (m, 1H), 0.47-0.31 (m, 1H); MS: m/z=466 (M+H); SFC retention time: 0.54 min.

152b: ¹H NMR (400 MHz, DMSO-d₆) δ 12.96-12.45 (m, 1H), 10.12-9.52 (m, 1H), 8.12 (s, 1H), 7.69-7.50 (m, 3H), 7.41-7.25 (m, 3H), 5.41-5.19 (m, 1H), 3.18-2.61 (m, 7H), 2.02-1.81 (m, 1H), 1.78-1.50 (m, 4H), 1.39 (s, 3H), 1.10-0.97 (m, 1H), 0.51-0.29 (m, 1H); MS: m/z=466 (M+H); SFC retention time: 0.72 min.

152c: ¹H NMR (400 MHz, DMSO-d₆) δ 12.95-12.48 (m, 1H), 10.09-9.49 (m, 1H), 8.12 (s, 1H), 7.66 (s, 1H), 7.58-7.50 (m, 2H), 7.40-7.26 (m, 3H), 5.39-5.22 (m, 1H), 3.20-2.55 (m, 7H), 2.01-1.84 (m, 1H), 1.75-1.51 (m, 4H), 1.39 (s, 3H), 1.08-0.97 (m, 1H), 0.48-0.30 (m, 1H); MS: m/z=466 (M+H); SFC retention time: 0.5 min.

152d: ¹H NMR (400 MHz, DMSO-d₆) δ 12.93-12.47 (m, 1H), 10.08-9.51 (m, 1H), 8.12 (s, 1H), 7.69-7.61 (m, 1H), 7.57-7.50 (m, 2H), 7.40-7.25 (m, 3H), 5.39-5.24 (m, 1H), 3.19-2.55 (m, 7H), 2.00-1.85 (m, 1H), 1.78-1.50 (m, 4H), 1.39 (s, 3H), 1.07-0.98 (m, 1H), 0.48-0.29 (m, 1H); MS: m/z=466 (M+H); SFC retention time: 1.2 min.

### Examples 153a and 153b

### N-(1-benzyl-1H-pyrazol-4-yl)-5a-methyl-1,4,5,5a,6,6a-hexahydrocyclopropa[g]indazole-3-carboxamide

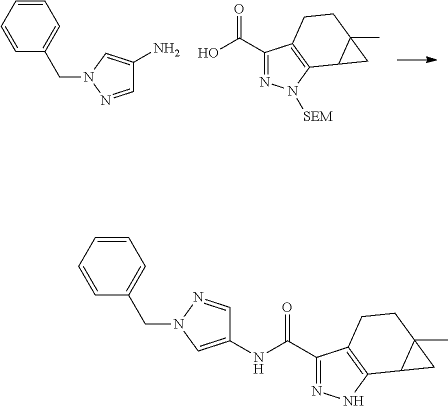 [SMILES: Nc1cnn(Cc2ccccc2)c1 ; CC12CCc3c(C(=O)O)nn(COCC[Si](C)(C)C)c3C1C2 ; CC12CCc3c(C(=O)Nc4cnn(Cc5ccccc5)c4)n[nH]c3C1C2]

Prepared in an analogous manner to N-(1-(3-cyanobenzyl)-1H-pyrazol-4-yl)-6-(1H-pyrazol-4-yl)-4,5,6,7-tetrahydro-1H-indazole-3-carboxamide (Examples 1a and 1b), replacing 1-((2-(trimethylsilyl)ethoxy)methyl)-6-(1-((2-(trimethylsilyl)ethoxy)methyl)-1H-pyrazol-4-yl)-4,5,6,7-tetrahydro-1H-indazole-3-carboxylic acid (Example C1) with 5a-methyl-1-((2-(trimethylsilyl)ethoxy)methyl)-1,4,5,5a,6,6a-hexahydrocyclopropa[g]indazole-3-carboxylic acid (Example C39) and 3-((4-amino-1H-pyrazol-1-yl)methyl)benzonitrile (Example A1) with 1-benzyl-1H-pyrazol-4-amine (Example A2).

SFC Conditions: ChiralPak AS (4.6×50 mm, 5 μm particle size) at 20% methanol w/0.1% NH₄OH; 5 mL/min, 120 bars, 40° C.

153a: ¹H NMR (400 MHz, DMSO-d₆) δ 12.94 (s, 1H), 10.03 (s, 1H), 8.05 (s, 1H), 7.63 (s, 1H), 7.37-7.19 (m, 5H), 5.27 (s, 2H), 3.05-2.95 (m, 1H), 2.24-2.09 (m, 1H), 2.01-1.90 (m, 1H), 1.77-1.68 (m, 1H), 1.58-1.44 (m, 1H), 1.26 (s, 3H), 0.91-0.79 (m, 2H); MS: m/z=348 (M+H); SFC retention time: 1.0 min.

153b: ¹H NMR (400 MHz, DMSO-d₆) δ 12.93 (s, 1H), 10.01 (s, 1H), 8.04 (s, 1H), 7.63 (s, 1H), 7.40-7.15 (m, 5H), 5.26 (s, 2H), 3.06-2.95 (m, 1H), 2.24-2.10 (m, 1H), 2.01-1.90 (m, 1H), 1.76-1.68 (m, 1H), 1.58-1.45 (m, 1H), 1.26 (s, 3H), 0.92-0.79 (m, 2H); MS: m/z=348 (M+H); SFC retention time: 1.8 min.

### Examples 154a-d

### 6,6-dimethyl-N-(1-((3-(methylsulfonyl)phenyl)(tetrahydro-2H-pyran-4-yl)methyl)-1H-pyrazol-4-yl)-4,5,6,7-tetrahydro-1H-indazole-3-carboxamide

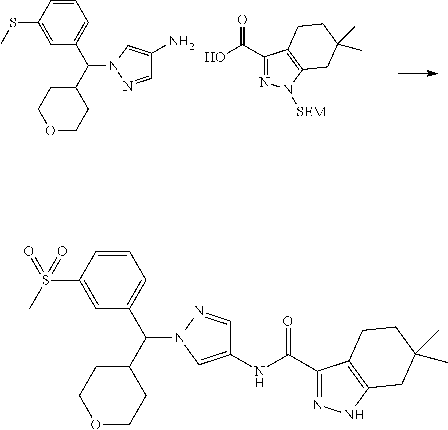

Prepared in an analogous manner to 6,6-dimethyl-N-(1-(2-(methylsulfonyl)-1-phenylethyl)-1H-pyrazol-4-yl)-4,5,6,7-tetrahydro-1H-indazole-3-carboxamide (Examples 64a and 64b), replacing 1-(2-methylsulfanyl-1-phenyl-ethyl)pyrazol-4-amine (Example A14) with 1-((3-(methylthio)phenyl)(tetrahydro-2H-pyran-4-yl)methyl)-1H-pyrazol-4-amine (Example A69).

SFC Conditions: Lux Cellulose 1 (4.6×50 mm, 5 μm particle size) at 25% methanol w/0.1% NH₄OH; 5 mL/min, 120 bars, 40° C.

154a: ¹H NMR (400 MHz, DMSO-d₆) δ 12.78 (s, 1H), 10.07 (s, 1H), 8.20 (s, 1H), 8.10 (t, J=1.8 Hz, 1H), 7.89 (ddt, J=21.6, 8.0, 1.2 Hz, 2H), 7.70 (s, 1H), 7.65 (t, J=7.8 Hz, 1H), 5.27 (d, J=10.7 Hz, 1H), 3.88-3.71 (m, 2H), 3.21 (s, 3H), 2.66 (dd, J=7.1, 5.2 Hz, 3H), 2.38 (s, 2H), 1.47 (t, J=6.4 Hz, 2H), 1.35-1.00 (m, 4H), 0.96 (s, 6H); MS: m/z=512 (M+H); SFC retention time 0.96 min.

154b: ¹H NMR (400 MHz, DMSO-d₆) δ 12.78 (s, 1H), 10.06 (s, 1H), 8.20 (d, J=2.4 Hz, 1H), 8.10 (t, J=1.9 Hz, 1H), 7.90 (dd, J=21.0, 7.7 Hz, 2H), 7.78-7.55 (m, 2H), 5.27 (d, J=10.7 Hz, 1H), 3.79 (d, J=13.9 Hz, 2H), 3.21 (d, J=2.3 Hz, 5H), 2.66 (d, J=7.3 Hz, 4H), 2.38 (s, 3H), 1.47 (t, J=6.4 Hz, 2H), 1.38-1.02 (m, 5H), 0.96 (d, J=2.5 Hz, 7H); MS: m/z=512.3 (M+H); SFC retention time 1.2 min.

### Biological Example

The ability of purified ITK (Invitrogen PV3875) to catalyze peptide phosphorylation is monitored using a Caliper LabChip 3000 microfluidic unit (Caliper assay) or by liquid chromatography-mass spectrometry (LCMS) using a Waters Acquity system (LCMS assay). In the Caliper assay, ITK is incubated at room temperature with test compounds for 45 minutes in 100 mM 2-[4-(2-hydroxyethyl)piperazin-1-yl]ethanesulfonic acid (HEPES) buffer (pH 7.2) containing 10 mM MgCl₂, 2 mM dithiothreitol (DTT), 20 μM adenosine-5′-triphosphate (ATP), 0.015% Brij 35, 2% dimethylsulfoxide (DMSO), and 2 μM (5-carboxyfluorescein)-EFPIYDFLPAKKK-NH₂ peptide substrate. Reactions are quenched by the addition of 2,2′,2″,2′″-(ethane-1,2-diyldinitrilo)tetraacetic acid (50 mM final). Non-phosphorylated substrate and phosphorylated product peptides are separated and quantified using a Caliper LabChip 3000 instrument. In the LCMS assay, ITK is incubated at room temperature with test compounds for 1 hour in 50 mM HEPES buffer (pH 7.2) containing 15 mM MgCl₂, 2 mM DTT, 20 M ATP, 0.015% Brij 35, 2% DMSO, and 2 μM Acetyl-EFPIYDFLPAKKK-NH₂ peptide substrate. Reactions are quenched by the addition of trichloroacetic acid (5% v/v final). Non-phosphorylated substrate and phosphorylated product peptides are separated by ultra performance LC and detected by a coupled triple quadrupole MS device applying multiple reaction monitoring (MRM) for quantification. The area of the MRM-extracted mass signal is used to assess the inhibition by test compounds. Equilibrium dissociation constant (Kᵢ) values for ITK inhibitors are calculated from plots of activity vs. inhibitor concentration using Morrison's quadratic equation that accounts for the potential of tight binding, and by also applying the conversion factor that accounts for competitive inhibition and the concentration of ATP used in the assay relative to its apparent Michaelis constant (K_{m,app}).

Examples 1-154b were tested in the above assay and found to have the activities given in Table 1.

TABLE 1

| Example Number | ITK Enzyme Ki (nM) |
|---|---|
| 1a | 9.4 |
| 1b | 8.3 |
| 2a | 68 |
| 2b | 34 |
| 3 | 31 |
| 4a | 360 |
| 4b | 240 |
| 5a | 310 |
| 5b | 380 |
| 6 | 5.3 |
| 7 | 69 |
| 8a | 70 |
| 8b | 77 |
| 9a | 83 |
| 9b | 22 |
| 10a | 1.0 |
| 10b | 15 |
| 11 | 10 |
| 12 | 75 |
| 13 | 240 |
| 14a | 120 |
| 14b | 21 |
| 15a | 11 |
| 15b | 100 |
| 16a | 0.5 |
| 16b | 7.9 |
| 17 | 36 |
| 18 | 180 |
| 19a | 130 |
| 19b | 7.9 |
| 20 | 89 |
| 21 | 7.4 |
| 22a | 190 |
| 22b | 37 |
| 23a | 24 |
| 23b | 550 |
| 24a | 9.4 |
| 24b | 3.5 |
| 25a | 1.7 |
| 25b | 94 |
| 26a | 2 |
| 26b | 46 |
| 26c | 22 |
| 26d | 780 |
| 27a | 30 |
| 27b | 11 |
| 27c | 45 |
| 27d | 96 |
| 28a | 16 |
| 28b | 6 |
| 28c | 155 |
| 28d | 98 |
| 29a | 200 |
| 29b | 3 |
| 30a | 2 |
| 30b | 4 |
| 31 | 260 |
| 32 | 38 |
| 33a | 1 |
| 33b | 3 |
| 34a | 3 |
| 34b | 0.7 |
| 35a | 13 |
| 35b | 2 |
| 35c | 220 |
| 35d | 190 |
| 36a | 0.7 |
| 36b | 3 |
| 37a | 14 |
| 37b | 2 |
| 38a | 4 |
| 38b | 0.5 |
| 39a | 0.7 |
| 39b | 6 |
| 40a | 0.2 |
| 40b | 3 |
| 41a | 18 |
| 41b | 0.8 |
| 42a | 10 |
| 42b | 0.9 |
| 43a | 0.2 |
| 43b | 6.2 |
| 44a | 6 |
| 44b | 0.2 |
| 45a | 370 |
| 45b | 48 |
| 46a | 6 |
| 46b | 27 |
| 47a | 2 |
| 47b | 9 |
| 48a | 180 |
| 48b | 280 |
| 49a | 10 |
| 49b | 2 |
| 50a | 260 |
| 50b | 620 |
| 51a | 160 |
| 51b | 860 |
| 52a | 0.2 |
| 52b | 17 |
| 52c | 2 |
| 52d | 100 |
| 53a | 5 |
| 53b | 29 |
| 54a | 1 |
| 54b | 6 |
| 55a | 5 |
| 55b | 18 |
| 56a | 9 |
| 56b | 13 |
| 57a | 2 |
| 57b | 12 |
| 58a | 7 |
| 58b | 0.6 |
| 59 | 40 |
| 60a | 0.3 |
| 60b | 7 |
| 61a | 5 |
| 61b | 1 |
| 62a | 3 |
| 62b | 16 |
| 63a | 0.1 |
| 63b | 1 |
| 64a | 0.5 |
| 64b | 9 |
| 65a | 42 |
| 65b | 56 |
| 66a | 0.3 |
| 66b | 1 |
| 67a | 0.2 |
| 67b | 3 |
| 68a | 0.2 |
| 68b | 4 |
| 69a | 2 |
| 69b | 0.7 |
| 70a | 11 |
| 70b | 7 |
| 71a | 4000 |
| 71b | 790 |
| 72 | 78 |
| 73a | 0.7 |
| 73b | 7 |
| 74a | 0.2 |
| 74b | 3 |
| 75a | 2 |
| 75b | 0.3 |
| 76 | 4 |
| 77a | 20 |
| 77b | 2 |
| 78a | 26 |
| 78b | 6 |
| 79a | 46 |
| 79b | 95 |
| 80a | 2 |
| 80b | 2 |
| 81a | 17 |
| 81b | 3 |
| 82 | 61 |
| 83a | <0.1 |
| 83b | 2 |
| 84a | 46 |
| 84b | 13 |
| 85a | 0.6 |
| 85b | 0.9 |
| 86a | 0.1 |
| 86b | 3 |
| 86c | 2 |
| 86d | 5 |
| 87a | 0.4 |
| 87b | 1 |
| 88a | 0.1 |
| 88b | 0.7 |
| 89a | 0.2 |
| 89b | 4 |
| 90a | 570 |
| 90b | 10 |
| 91a | 11 |
| 91b | 19 |
| 92a | 7 |
| 92b | 10 |
| 93a | <0.1 |
| 93b | 1 |
| 94a | 0.3 |
| 94b | 13 |
| 95a | 5 |
| 95b | 320 |
| 96a | 57 |
| 96b | 2 |
| 97a | 2 |
| 97b | 6 |
| 98a | 8 |
| 98b | 2 |
| 99a | 4 |
| 99b | 4 |
| 100a | 17 |
| 100b | 7 |
| 101a | 41 |
| 101b | 12 |
| 102a | 300 |
| 102b | 10 |
| 103a | 20 |
| 103b | 9 |
| 104a | 36 |
| 104b | 8 |
| 105a | 3 |
| 105b | 240 |
| 106a | 3 |
| 106b | 5 |
| 107a | 18 |
| 107b | 14 |
| 108a | 17 |
| 108b | 5 |
| 109a | 4 |
| 109b | 21 |
| 110a | 0.2 |
| 110b | 3 |
| 111 | 8 |
| 112 | 6 |
| 113a | 5 |
| 113b | 0.3 |
| 114a | 0.8 |
| 114b | 12 |
| 115a | 0.2 |
| 115b | 7 |
| 116a | <0.1 |
| 116b | 2 |
| 117a | 0.2 |
| 117b | 6 |
| 118 | 34 |
| 119a | 0.1 |
| 119b | 4 |
| 120a | 0.1 |
| 120b | <0.1 |
| 121a | 6 |
| 121b | 1 |
| 122 | 4 |
| 123a | 88 |
| 123b | 4000 |
| 124 | 140 |
| 125a | 2 |
| 125b | 10 |
| 126a | 2 |
| 126b | 0.1 |
| 126c | 4 |
| 126d | 0.2 |
| 127a | 0.2 |
| 127b | 6 |
| 128a | 42 |
| 128b | 6 |
| 129a | 0.7 |
| 129b | 6 |
| 130a | 25 |
| 130b | 2 |
| 131a | 1 |
| 131b | 11 |
| 132a | 77 |
| 132b | 10 |
| 133a | 0.4 |
| 133b | 2 |
| 134a | 0.3 |
| 134b | 5 |
| 134c | 10 |
| 134d | 0.3 |
| 135a | 130 |
| 135b | 14 |
| 136a | 0.5 |
| 136b | 0.9 |
| 136c | 6 |
| 136d | 9 |
| 137a | 5 |
| 137b | 0.2 |
| 137c | 3 |
| 137d | 11 |
| 138a | >400 |
| 138b | 0.3 |
| 139a | 1 |
| 139b | 0.7 |
| 139c | 12 |
| 139d | 12 |
| 139e | 1 |
| 139f | 0.6 |
| 139g | 8 |
| 139h | 12 |
| 140a | 0.4 |
| 140b | 14 |
| 141 | <0.1 |
| 142a | <0.1 |
| 142b | <0.1 |
| 143a | <0.1 |
| 143b | 2 |
| 143c | 2 |
| 143d | <0.1 |
| 144a | 0.1 |
| 144b | 3 |
| 144c | 0.1 |
| 144d | 2 |
| 145a | <0.1 |
| 145b | 0.3 |
| 145c | <0.1 |
| 145d | 0.3 |
| 146a | 0.1 |
| 146b | 2 |
| 146c | 0.1 |
| 146d | 2 |
| 147a | 0.6 |
| 147b | 4 |
| 147c | 7 |
| 147d | 0.6 |
| 148a | >400 |
| 148b | >400 |
| 149a | >400 |
| 149b | >400 |
| 150a | 130 |
| 150b | 32 |
| 151a | 16 |
| 151b | 12 |
| 152a | 42 |
| 152b | 3 |
| 152c | 2 |
| 152d | 15 |
| 153a | 58 |
| 153b | 15 |
| 154a | 0.1 |
| 154b | 1 |

We claim:

1. A compound of formula (AA): 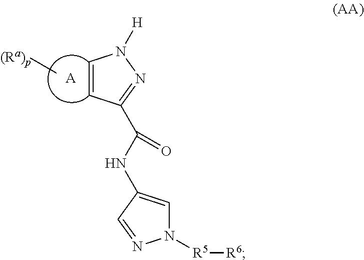
or stereoisomers or a pharmaceutically acceptable salt thereof, wherein:
ring A is a 5-7-membered cycloalkyl or 5-7-membered heterocyclyl;
p is 0, 1, 2, 3, 4, 5, 6, 7 or 8;
each R^{a} is independently a bond, hydrogen, C₁-C₁₂ alkyl, C₂-C₁₂ alkenyl, C₂-C₁₂ alkynyl, C₁-C₆ alkylene, C₂-C₆ alkenylene, C₂-C₆ alkynylene, halogen, —CN, —OR⁷, —SR⁷, —NR⁷R⁸, —CF₃, —CHF₂, —CH₂F, —OCF₃, —NO₂, —C(O)R⁷, —C(O)OR⁷, —C(O)NR⁷R⁸, —NR⁷C(O)R⁸, —S(O)₁₋₂R⁷, —NR⁷S(O)₁₋₂R⁸, —S(O)₁₋₂NR⁷R⁸, C₃-C₆ cycloalkyl, 3-10-membered heterocyclyl or 6-10 membered aryl, wherein each R^{a}, other than a bond and hydrogen, are independently optionally substituted by R⁹, or
two R^{a} are taken together with the atoms to which they are attached to form a C₁-C₆ alkylene, C₂-C₆ alkenylene, C₂-C₆ alkynylene, C₃-C₆ cycloalkyl, 3-10-membered heterocyclyl or 6-10 membered aryl, wherein said cycloalkyl, heterocyclyl and aryl are independently optionally substituted by R⁹, or
two R^{a} are taken together with the atom to which they are attached to form a C₃-C₆ cycloalkyl or 3-10-membered heterocyclyl, wherein said cycloalkyl and heterocyclyl are independently optionally substituted by R⁹;
R⁵ is hydrogen, C₁-C₆ alkylene, C₂-C₆ alkenylene, C₂-C₆ alkynylene, or 3-10-membered heterocyclene wherein said alkylene, alkenylene, alkynylene and heterocyclene are independently optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —CHF₂, —CH₂F, —OCF₃, C₃-C₆ cycloalkyl, 3-10-membered heterocyclyl or 6-10 membered aryl, and wherein said alkyl, alkenyl, alkynyl, cycloalkyl, heterocyclyl and aryl are independently optionally substituted by R²⁰;
R⁶ is hydrogen, C₃-C₁₀ cycloalkyl, 3-10-membered heterocyclyl or 6-10-membered aryl, wherein R⁶ is independently optionally substituted by R⁹, or R₆ is absent when R₅ is hydrogen;
each R⁷ and R⁸ are independently hydrogen, C₁-C₆ alkyl, C₃-C₆ cycloalkyl, 3-6-membered heterocyclyl or phenyl, wherein said alkyl, cycloalkyl, heterocyclyl and phenyl are independently optionally substituted by halogen, —CN, —CF₃, —CHF₂, —CH₂F, —OCF₃ or OXO; or
R⁷ and R⁸ are independently taken together with the atom to which they are attached to form a 3-6 membered heterocyclyl optionally substituted by halogen, oxo or C₁-C₆ alkyl optionally substituted by halogen or oxo;
each R⁹ is independently hydrogen, oxo, C₁-C₁₂ alkyl, C₂-C₁₂ alkenyl, C₂-C₁₂ alkynyl, halogen, —(C₀-C₆ alkylene)CN, —(C₀-C₆ alkylene)OR¹⁰, —(C₀-C₆ alkylene)SR¹⁰, —(C₀-C₆ alkylene)NR¹⁰R¹¹, —(C₀-C₆ alkylene)CF₃, —(C₀-C₆ alkylene)NO₂, —(C₀-C₆ alkylene)C(O)R¹⁰, —(C₀-C₆ alkylene)C(O)OR¹⁰, —(C₀-C₆ alkylene)C(O)NR¹⁰R¹¹, —(C₀-C₆ alkylene)NR¹⁰C(O)R¹¹, —(C₀-C₆ alkylene)S(O)₁₋₂R¹⁰, —(C₀-C₆ alkylene)NR¹⁰S(O)₁₋₂R¹¹, —(C₀-C₆ alkylene)S(O)₁₋₂NR¹⁰R¹¹, —(C₀-C₆ alkylene)(C₃-C₆ cycloalkyl), —(C₀-C₆ alkylene)(3-10-membered heterocyclyl), —(C₀-C₆ alkylene)C(O)(3-10-membered heterocyclyl), or —(C₀-C₆ alkylene)(6-10 membered aryl), wherein each R⁹, other than hydrogen, is independently optionally substituted by halogen, oxo, —CF₃, —CN, —OR¹², —SR¹², —NR²R³, —C(O)R¹², —S(O)₁₋₂R¹², C₁-C₆ alkyl optionally substituted by oxo or halogen, C₂-C₆ alkenyl optionally substituted by oxo or halogen, or C₂-C₆ alkynyl optionally substituted by oxo or halogen;
each R¹⁰ and R¹¹ are independently hydrogen, C₁-C₆ alkyl, C₂-C₆ alkenyl, C₂-C₆ alkynyl, 3-6-membered heterocyclyl, phenyl or C₃-C₆ cycloalkyl, wherein said alkyl, alkenyl, alkynyl, heterocyclyl, phenyl and cycloalkyl are independently optionally substituted by halogen, oxo, —CF₃, —OCF₃, —OR¹⁴, —SR¹⁴, —NR¹⁴R¹⁵, —CN, 3-6-membered heterocyclyl, phenyl, C₃-C₆ cycloalkyl or C₁-C₆ alkyl optionally substituted by halogen or oxo; or
R¹⁰ and R¹¹ are independently taken together with the atom to which they are attached to form a 3-6 membered heterocyclyl optionally substituted by halogen, oxo or C₁-C₆ alkyl optionally substituted by halogen or oxo;
each R¹² and R¹³ are independently hydrogen or C₁-C₆ alkyl optionally substituted by halogen or oxo; or
R¹² and R¹³ are independently taken together with the atom to which they are attached to form a 3-6 membered heterocyclyl optionally substituted by halogen, oxo or C₁-C₆ alkyl optionally substituted by halogen;
each R¹⁴ and R¹⁵ are independently hydrogen or C₁-C₆ alkyl optionally substituted by halogen or oxo; or
R¹⁴ and R¹⁵ are independently taken together with the atom to which they are attached to form a 3-6 membered heterocyclyl optionally substituted by halogen, oxo or C₁-C₆ alkyl optionally substituted by halogen;
each R¹⁶ and R¹⁷ are independently hydrogen, —S(O)₁₋₂C₁-C₆ alkyl, C₁-C₆ alkyl, C₂-C₆ alkenyl, C₂-C₆ alkynyl, 3-6-membered heterocyclyl, phenyl or C₃-C₆ cycloalkyl, wherein said alkyl, alkenyl, alkynyl, heterocyclyl, phenyl and cycloalkyl are independently optionally substituted by halogen, oxo, —CF₃, —OCF₃, —OR¹⁸, —SR¹⁸, —NR¹⁸R¹⁹, —CN, 3-6-membered heterocyclyl, phenyl, C₃-C₆ cycloalkyl or C₁-C₆ alkyl optionally substituted by halogen or oxo; or
R¹⁶ and R¹⁷ are independently taken together with the atom to which they are attached to form a 3-6 membered heterocyclyl optionally substituted by halogen, oxo or C₁-C₆ alkyl optionally substituted by halogen or oxo;
each R¹⁸ and R¹⁹ are independently hydrogen or C₁-C₆ alkyl optionally substituted by halogen or oxo; or
R¹⁸ and R¹⁹ are independently taken together with the atom to which they are attached to form a 3-6 membered heterocyclyl optionally substituted by halogen, oxo or C₁-C₆ alkyl optionally substituted by halogen;
each R²⁰ is independently hydrogen, oxo, C₁-C₁₂ alkyl, C₂-C₁₂ alkenyl, C₂-C₁₂ alkynyl, halogen, —(C₀-C₆ alkylene)CN, —(C₀-C₆ alkylene)OR²¹, —(C₀-C₆ alkylene)SR²¹, —(C₀-C₆ alkylene)NR²¹R²², —(C₀-C₆ alkylene)CF₃, —(C₀-C₆ alkylene)NO₂, —(C₀-C₆ alkylene)C(O)R²¹, —(C₀-C₆ alkylene)C(O)OR²¹, —(C₀-C₆ alkylene)C(O)NR²¹R²², —(C₀-C₆ alkylene)NR²¹C(O)R²², —(C₀-C₆ alkylene)S(O)₁₋₂R²¹, —(C₀-C₆ alkylene)NR²¹S(O)₁₋₂R²², —(C₀-C₆ alkylene)S(O)₁₋₂NR²¹R²², —(C₀-C₆ alkylene)(C₃-C₆ cycloalkyl), —(C₀-C₆ alkylene)(3-10-membered heterocyclyl), —(C₀-C₆ alkylene)C(O)(3-10-membered heterocyclyl), or —(C₀-C₆ alkylene)(6-10 membered aryl), wherein each R²⁰, other than hydrogen, is independently optionally substituted by halogen, oxo, —CF₃, —CN, —OH or C₁-C₆ alkyl optionally substituted by oxo or halogen; and
each R²¹ and R²² are independently hydrogen, C₁-C₆ alkyl or 3-6 membered heterocyclyl wherein said alkyl or heterocyclyl is optionally substituted by halogen or oxo; or
R²¹ and R²² are independently taken together with the atom to which they are attached to form a 3-6 membered heterocyclyl optionally substituted by halogen, oxo or C₁-C₆ alkyl optionally substituted by halogen.

2. The compound of claim 1, having formula (II): 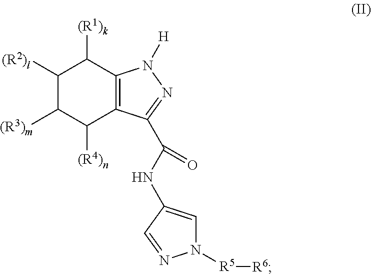
stereoisomers or a pharmaceutically acceptable salt thereof, wherein:
k, l, m and n are independently 0, 1 or 2; and
each R¹, R², R³ and R⁴ are independently a bond, hydrogen, C₁-C₁₂ alkyl, C₂-C₁₂ alkenyl, C₂-C₁₂ alkynyl, C₁-C₆ alkylene, C₂-C₆ alkenylene, C₂-C₆ alkynylene, halogen, —CN, —OR⁷, —SR⁷, —NR⁷R⁸, —CF₃, —CHF₂, —CH₂F, —OCF₃, —NO₂, —C(O)R⁷, —C(O)OR⁷, —C(O)NR⁷R⁸, —NR⁷C(O)R⁸, —S(O)₁₋₂R⁷, —NR⁷S(O)₁₋₂R⁸, —S(O)₁₋₂NR⁷R⁸, C₃-C₆ cycloalkyl, 3-10 membered heterocyclyl or 6-10 membered aryl, wherein each R¹, R², R³ and R⁴, other than a bond and hydrogen, are independently optionally substituted by R⁹, or
one R¹ and one of R², R³ and R⁴ are taken together with the atoms to which they are attached to form a C₁-C₆ alkylene, C₂-C₆ alkenylene, C₂-C₆ alkynylene, C₃-C₆ cycloalkyl, 3-10 membered heterocyclyl or 6-10 membered aryl, wherein said cycloalkyl, heterocyclyl and aryl are independently optionally substituted by R⁹, or
one R² and one of R¹, R³ and R⁴ are taken together with the atoms to which they are attached to form a C₁-C₆ alkylene, C₂-C₆ alkenylene, C₂-C₆ alkynylene, C₃-C₆ cycloalkyl, 3-10 membered heterocyclyl or 6-10 membered aryl, wherein said cycloalkyl, heterocyclyl and aryl are independently optionally substituted by R⁹, or
one R³ and one of R¹, R² and R⁴ are taken together with the atoms to which they are attached to form a C₁-C₆ alkylene, C₂-C₆ alkenylene, C₂-C₆ alkynylene, C₃-C₆ cycloalkyl, 3-10 membered heterocyclyl or 6-10 membered aryl, wherein said cycloalkyl, heterocyclyl and aryl are independently optionally substituted by R⁹, or
one R⁴ and one of R¹, R² and R³ are taken together with the atoms to which they are attached to form a C₁-C₆ alkylene, C₂-C₆ alkenylene, C₂-C₆ alkynylene, C₃-C₆ cycloalkyl, 3-10 membered heterocyclyl or 6-10 membered aryl, wherein said cycloalkyl, heterocyclyl and aryl are independently optionally substituted by R⁹, or
two R¹ are taken together with the atom to which they are attached to form a C₃-C₆ cycloalkyl or 3-10 membered heterocyclyl, wherein said cycloalkyl and heterocyclyl are independently optionally substituted by R⁹, or
two R² are taken together with the atom to which they are attached to form a C₃-C₆ cycloalkyl or 3-10 membered heterocyclyl, wherein said cycloalkyl and heterocyclyl are independently optionally substituted by R⁹, or
two R³ are taken together with the atom to which they are attached to form a C₃-C₆ cycloalkyl or 3-10 membered heterocyclyl, wherein said cycloalkyl and heterocyclyl are independently optionally substituted by R⁹, or
two R⁴ are taken together with the atom to which they are attached to form a C₃-C₆ cycloalkyl or 3-10 membered heterocyclyl, wherein said cycloalkyl and heterocyclyl are independently optionally substituted by R⁹.

3. The compound of claim 2, wherein each R¹, R², R³ and R⁴ are independently a bond, hydrogen, C₁-C₁₂ alkyl, C₁-C₆ alkylene, halogen, —OR⁷, C₃-C₆ cycloalkyl or 3-10 membered heterocyclyl, wherein each R¹, R², R³ and R⁴, other than a bond and hydrogen, are independently optionally substituted by R⁹, or
one R¹ and one R⁴ are taken together with the atoms to which they are attached to form a C₁-C₆ alkylene, independently optionally substituted by R⁹, or
one R¹ and one R³ are taken together with the atoms to which they are attached to form a C₁-C₆ alkylene, independently optionally substituted by R⁹, or
one R² and one R⁴ are taken together with the atoms to which they are attached to form a C₁-C₆ alkylene, independently optionally substituted by R⁹, or
one R¹ and one R² are taken together with the atoms to which they are attached to form a C₃-C₆ cycloalkyl independently optionally substituted by R⁹, or
one R² and one R³ are taken together with the atoms to which they are attached to form a C₃-C₆ cycloalkyl independently optionally substituted by R⁹, or
one R³ and one R⁴ are taken together with the atoms to which they are attached to form a C₃-C₆ cycloalkyl independently optionally substituted by R⁹, or
two R² are taken together with the atom to which they are attached to form a C₃-C₆ cycloalkyl or 3-10 membered heterocyclyl, wherein said cycloalkyl and heterocyclyl are independently optionally substituted by R⁹, or
two R³ are taken together with the atom to which they are attached to form a C₃-C₆ cycloalkyl or 3-10 membered heterocyclyl, wherein said cycloalkyl and heterocyclyl are independently optionally substituted by R⁹.

4. The compound of claim 2, wherein each R¹, R², R³ and R⁴ are independently a bond, hydrogen, methyl, ethyl, methylene, ethylene, fluoro, —OH, —OCH₃, —CH₂OH, cyclopropyl, pyrazolo, pyrimidinyl, oxetanyl or tetrahydrofuranyl, wherein each R¹, R², R³ and R⁴, other than a bond and hydrogen, are independently optionally substituted by R⁹, or
one R¹ and one R⁴ are taken together with the atoms to which they are attached to form a methylene or ethylene, independently optionally substituted by R⁹, or
one R¹ and one R³ are taken together with the atoms to which they are attached to form a methylene, independently optionally substituted by R⁹, or
one R² and one R⁴ are taken together with the atoms to which they are attached to form a ethylene, independently optionally substituted by R⁹, or
one R¹ and one R² are taken together with the atoms to which they are attached to form a C₃ cycloalkyl independently optionally substituted by R⁹, or
one R² and one R³ are taken together with the atoms to which they are attached to form a C₃ cycloalkyl independently optionally substituted by R⁹, or
one R³ and one R⁴ are taken together with the atoms to which they are attached to form a C₃ cycloalkyl independently optionally substituted by R⁹, or
two R² are taken together with the atom to which they are attached to form a C₃ cycloalkyl, oxetanyl or tetrahydrofuranyl, each independently optionally substituted by R⁹, or
two R³ are taken together with the atom to which they are attached to form a C₃ cycloalkyl, oxetanyl or tetrahydrofuranyl, each independently optionally substituted by R⁹.

5. The compound of claim 2, wherein R² is independently 3-10 membered heterocyclyl independently optionally substituted by R⁹.

6. The compound of claim 2, R² is independently C₁-C₁₂ alkyl independently optionally substituted by R⁹.

7. The compound of claim 2, wherein two R² are taken together with the atom to which they are attached to form a C₃-C₆ cycloalkyl or 3-10 membered heterocyclyl, wherein said cycloalkyl and heterocyclyl are independently optionally substituted by R⁹.

8. The compound of claim 2, wherein R² is independently 3-10-membered heterocyclyl independently optionally substituted by R⁹.

9. The compound of claim 2, wherein R³ is independently C₁-C₁₂ alkyl independently optionally substituted by R⁹.

10. The compound of claim 2, wherein two R³ are taken together with the atom to which they are attached to form a C₃-C₆ cycloalkyl or 3-10 membered heterocyclyl, wherein said cycloalkyl and heterocyclyl are independently optionally substituted by R⁹.

11. The compound of claim 1, wherein R⁵ is 3-10 membered heterocyclene optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —CHF₂, —CH₂F, —OCF₃, C₃-C₆ cycloalkyl, 3-10-membered heterocyclyl or 6-10 membered aryl, and wherein said alkyl, alkenyl, alkynyl, cycloalkyl, heterocyclyl and aryl are independently optionally substituted by R²⁰.

12. The compound of claim 1, wherein R⁵ is C₁-C₆ alkylene optionally substituted by halogen, oxo, C₁-C₁₂ alkyl, C₂-C₁₂ alkenyl, C₂-C₁₂ alkynyl, —OR¹⁶, —SR¹⁶, —NR¹⁶R¹⁷, —CN, —CF₃, —OCF₃, 3-10 membered heterocyclyl or 6-10 membered aryl, wherein said alkyl, alkenyl, alkynyl, heterocyclyl and aryl are independently optionally substituted by R²⁰.

13. The compound of claim 1, wherein R⁶ is 1,1-dioxothianyl, 1-oxothianyl, pyridinyl or phenyl independently optionally substituted by R⁹.

14. The compound of claim 13, wherein R⁹ is C₁-C₆ alkyl or oxo, wherein said alkyl is optionally substituted by R²⁰.

15. The compound of claim 1, wherein each R⁷ and R⁸ are independently hydrogen or methyl.

16. The compound of claim 1, wherein each R⁹ is independently hydrogen, C₁-C₁₂ alkyl, C₂-C₁₂ alkynyl, halogen, —CN, —(C₀-C₆ alkylene)OR¹⁰, —(C₀-C₆ alkylene)NR¹⁰R¹¹, —CF₃, —(C₀-C₆ alkylene)C(O)OR¹⁰, —(C₀-C₆ alkylene)C(O)NR¹⁰R¹¹, —(C₀-C₆ alkylene)(5-6 membered heterocyclyl), —(C₀-C₆ alkylene)C(O)(5-6 membered heterocyclyl) or phenyl, wherein each R⁹ is independently optionally substituted by halogen, oxo, —CF₃, —CN, —OR¹², —SR¹², —NR¹²R¹³, —C(O)R¹², —S(O)₁₋₂R¹², C₁-C₆ alkyl optionally substituted by oxo or halogen, C₂-C₆ alkenyl optionally substituted by oxo or halogen, or C₂-C₆ alkynyl optionally substituted by oxo or halogen.

17. The compound of claim 1, selected from Examples 1-154b.

18. A pharmaceutical composition comprising a compound of claim 1, a stereoisomer or a pharmaceutically acceptable salt thereof, and a therapeutically inert carrier, diluent or excipient.

19. A method of treating a disease mediated by ITK kinase, comprising administering an effective amount of a compound of claim 1, a stereoisomer or a pharmaceutically acceptable salt thereof to a mammal in need thereof.

20. A method of treating an inflammatory disease, comprising administering an effective amount of a compound of claim 1, a stereoisomer or a pharmaceutically acceptable salt thereof to a mammal in need thereof.

21. A process for manufacturing a compound of claim 1, comprising contacting a compound of formula (i), or salt thereof, with a compound of formula (ii), or salt thereof: 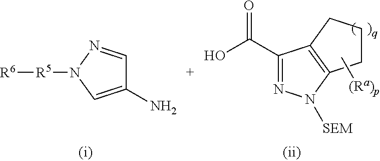
to form a compound of formula (AA) or salt thereof.
